(19) United States
(12) Patent Application Publication
(10) Pub. No.: US 2026/0345767 A1
(43) Pub. Date: Nov. 5, 2026

(54) VACCINES AGAINST CORONAVIRUSES

(71) Applicants: The Henry M. Jackson Foundation for the Advancement of Military Medicine, Inc., Bethesda, MD (US); The Government of the United States, as represented by the Secretary of the Army, Fort Detrick, MD (US); Sanofi Pasteur Inc., Swiftwater, PA (US); United States of America, as represented by the Secretary, Department of Health and Human Services, Rockville, MD (US)

(72) Inventors: Michael Gordon Joyce, Silver Spring, MD (US); Kayvon Modjarrad, Silver Spring, MD (US); Paul Thomas, Silver Spring, MD (US); Wei-Hung Chen, Silver Spring, MD (US); Agnes Hajduczki, Silver Spring, MD (US); Morgane Rolland, Silver Spring, MD (US); Eric Lewitus, Silver Spring, MD (US); Natalie Anosova, Waltham, MA (US); Nicholas Clark, Waltham, MA (US); Philip Davidson, Waltham, MA (US); Valerie Lecouturier, Paris (FR); Irina V. Ustyugova, Waltham, MA (US); William Warren, Waltham, MA (US); Monica Z. Wu, Waltham, MA (US); Daniel Douek, Rockville, MD (US); Richard Koup, Washington, DC (US)

(73) Assignees: The Henry M. Jackson Foundation for the Advancement of Military Medicine, Inc., Bethesda, MD (US); The Government of the United States, as represented by the Secretary of the Army, Fort Detrick, MD (US); Sanofi Pasteur Inc., Swiftwater, PA (US); United States of America, as represented by the Secretary, Department of Health and Human Services, Rockville, MD (US)

(21) Appl. No.: 19/105,579

(22) PCT Filed: Aug. 18, 2023
(86) PCT No.: PCT/US2023/030602

§ 371 Date: Feb. 21, 2025

Related U.S. Application Data
(60) Provisional application No. 63/399,990, filed on Aug. 22, 2022.
(60) Provisional application No. 63/400,334, filed on Aug. 23, 2022.
(60) Provisional application No. 63/431,286, filed on Dec. 8, 2022.

(30) Foreign Application Priority Data
Aug. 17, 2023 (EP) 23315318.8

(51) Int. Cl. A61K 39/215; A61K 9/51; A61K 39/00; A61P 37/04

(52) U.S. Cl. CPC A61K 39/215; A61K 9/5169; A61P 37/04; A61K 2039/53; A61K 2039/575; A61K 2039/6068; A61K 2039/70

(57) ABSTRACT

The present disclosure relates to the field of vaccines and binding molecules, as well as preparations and methods of their use in the treatment and/or prevention of disease. Described are vaccines and binding molecules, compositions containing the same, and uses thereof for treating or preventing coronavirus infections, including multivalent miRNA and nanoparticle vaccines.

### CROSS-REFERENCE TO RELATED APPLICATIONS

This application claims priority to U.S. Provisional Application No. 63/399,990, filed Aug. 22, 2022; U.S. Provisional Application No. 63/400,334, filed Aug. 23, 2022; U.S. Provisional Application No. 63/431,286, filed Dec. 8, 2022; and European Application 23315318.8, filed Aug. 17, 2023, the entire contents of each of which are incorporated herein by reference.

### GOVERNMENT SUPPORT CLAUSE

This invention was made with government support under W81XWH-18-2-0040 awarded by the United States Army Medical Research and Development Command, and MI220230 awarded by the Military Infectious Diseases Research Program. The government has certain rights in the invention.

### FIELD OF INVENTION

The present disclosure relates to the field of vaccines, as well as preparations and methods of their use in the treatment and/or prevention of disease. Described are vaccines, pharmaceutical compositions containing the same (also referred to herein as immunogenic compositions), and uses thereof for treating or preventing coronavirus infections and related viral infections, such as those caused by sarbecoviruses and merbecoviruses.

### BACKGROUND

The following discussion is merely provided to aid the reader in understanding the disclosure and is not admitted to describe or constitute prior art thereto.

The emergence of SARS-CoV-2—also named COVID-19 and sometimes referred to herein as SARS2 or SARS-2—marks the seventh coronavirus to be isolated from humans, and the third to cause a severe disease, after severe acute respiratory syndrome (SARS) and Middle East respiratory syndrome (MERS). The rapid spread and relatively high case fatality ratio of SARS-CoV-2 has resulted in significant loss of life and long-term morbidity for millions around the world. The rapidly evolving epidemiology of the SARS-CoV-2 pandemic underscored the need to elucidate the molecular biology of this novel coronavirus, particularly as new variants of the virus with differing levels of pathogenicity and transmissibility continue to spread.

Effective prophylactic vaccines against SARS-CoV-2 and other members of the sarbecovirus subgenus are urgently needed, to ensure protection against SARS-CoV-2 variants of concern, which continue to evolve and cause new outbreaks. Currently available vaccines against SARS-CoV-2 include mRNA, vectored, and recombinant protein vaccines directed to SARS-CoV-2 spike proteins from the parental, beta- and/or omicron strains. Despite these available vaccines, variants of SARS-CoV-2 continue to circulate amongst both vaccinated and unvaccinated individuals. This highlights the public health need for a vaccine that will be efficacious against current and future circulating SARS-CoV-2 variants. The development of a vaccine that elicits broad protective immune responses against sarbecoviruses could form the basis of a next-generation prophylactic vaccine. Vaccines against MERS-CoV and other merbecoviruses also are needed for pandemic prevention efforts. MERS-CoV continues to cause significant mortality and morbidity in the Arabian Peninsula, and, despite its currently low reproduction rate, the potential for a large pandemic outbreak remains high. Vaccines that can elicit broad protective immune responses against several or all beta-coronaviruses, and eventually several or all beta-coronaviruses, and/or a broad spectrum of sarbecoviruses are of interest for pandemic preparedness against other future coronavirus zoonotic events. The present disclosure provides mRNA and nanoparticle vaccines that can be used to treat or prevent coronavirus infections and other related viral infections.

### SUMMARY

Described herein are immunogenic compositions and vaccines for the treatment and/or prevention of infections caused by coronaviruses including merbecoviruses, and sarbecoviruses, and methods and uses of the same.

In accordance with one aspect, there are provided immunogenic compositions comprising at least two antigenic coronavirus peptides comprising at least a first antigenic coronavirus peptide and a second antigenic coronavirus peptide, or one or more messenger RNA (mRNA) molecules encoding them, wherein each antigenic coronavirus peptide is independently selected from: a receptor-binding domain (RBD or R) of a coronavirus, or a fragment or variant thereof, an N-terminal domain (NTD) of a coronavirus, or a fragment or variant thereof, an S1 domain of a coronavirus, or a fragment or variant thereof, a stabilized spike S-2P domain of a coronavirus, or a fragment or variant thereof, a stabilized spike S domain of a coronavirus, or a fragment or variant thereof, and a stabilized spike S-trimer of a coronavirus, or a fragment or variant thereof, wherein the antigenic coronavirus peptides include antigenic coronavirus peptides selected from the following combinations of strains: (i) two or more selected clade 1b; (ii) one or more selected from clade 1b and one or more selected from clade 1a (iii) one or more selected from clade 1b, and one or more selected from clade 1a and one or more selected from clade 2; (iv) one or more selected from clade 1b, and one or more selected from clade 1a, and one or more selected from clade 3; (v) one or more selected from clade 1b, one or more selected from clade 2, and one or more selected from clade 3; and (v) one or more selected from clade 1a, one or more selected from clade 2, and one or more selected from clade 3. In some embodiments, at least one of the antigenic coronavirus peptides is an S-2P peptide, optionally wherein the S-2P peptide comprises the amino acid sequence of any one of SEQ ID NOs 536-543, or a sequence having at least 80%, at least 85%, at least 90%, at least 95%, at least 96%, at least 97%, at least 98%, or at least 99% homology or identity thereto. Additionally or alternatively, in some embodiments, one or more of the antigenic coronavirus peptides is comprised in a fusion protein comprising the antigenic coronavirus peptide and a nanoparticle-forming protein, wherein two or more of said antigenic coronavirus peptides may be comprised in the same or different fusion proteins with a nanoparticle-forming protein.

In accordance with other aspects, there are provided immunogenic compositions comprising a nanoparticle comprising at least two antigenic coronavirus peptides comprising at least a first antigenic coronavirus peptide and a second antigenic coronavirus peptide, or one or more messenger RNA (mRNA) molecules encoding the at least two antigenic coronavirus peptides, wherein each antigenic coronavirus peptide is independently selected from a receptor-binding domain (RBD or R) of a coronavirus, or a fragment or variant thereof; an N-terminal domain (NTD) of a coronavirus, or a fragment or variant thereof; an S1 domain of a coronavirus, or a fragment or variant thereof; a stabilized extracellular spike S-2P domain of a coronavirus, or a fragment or variant thereof; a stabilized extracellular spike S domain of a coronavirus, or a fragment or variant thereof, and a stabilized extracellular spike S-trimer of a coronavirus, or a fragment or variant thereof, wherein each antigenic coronavirus peptide is comprised in a fusion protein comprising the antigenic coronavirus peptide and a nanoparticle-forming protein, wherein the antigenic coronavirus peptides may be comprised in the same or different fusion proteins, wherein the composition comprises antigenic coronavirus peptides from at least two different coronavirus strains, or one or more mRNA molecules encoding them. In some embodiments, each antigenic coronavirus peptide is from a coronavirus strain independently selected from clade 1a, clade 1b, clade 2, clade 3, and Middle East respiratory syndrome-related coronavirus (MERS-CoV), optionally wherein at least the first and second antigenic coronavirus peptides are from coronavirus strains of different clades. In some embodiments, at least the first and second antigenic coronavirus peptides are from different coronavirus strains independently selected from WA-1, Beta, Omicron BQ.1.1, Omicron XBB.1.5, a strain of SARS-CoV-1, BANAL20-247, Khosta-2, and MERS-CoV. In some embodiments, the antigenic coronavirus peptides include antigenic coronavirus peptides selected from the following combinations of strains: (i) two or more selected from WA-1, Beta, Omicron XBB.1.5, and Omicron BQ.1.1; (ii) one or more selected from WA-1, Beta, Omicron XBB.1.5, and Omicron BQ.1.1, and strains of SARS-CoV-1; (iii) one or more selected from WA-1, Beta, Omicron XBB.1.5, and Omicron BQ.1.1, and one or more selected from strains of SARS-CoV-1, BANAL20-247, and Khosta-2; (iv) one or more selected from WA-1, Beta, Omicron XBB.1.5, and Omicron BQ.1.1, and one or more selected from strains of SARS-CoV-1, BANAL20-247, and Khosta-2, and MERS-CoV; and (v) two or more selected from strains of SARS-CoV-1, BANAL20-247, and Khosta-2.

In some embodiments of any of the preceding aspects, the antigenic coronavirus peptides include antigenic coronavirus peptides selected from the following combinations of strains: WA-1, Beta, and Omicron BQ1.1; WA-1, Omicron BQ.1.1, and SARS-CoV-1; WA-1, SARS-CoV-1, and Khosta2; WA-1, SARS-CoV-1, and BANAL20-247; WA-1, SARS-CoV-1, and MERS-CoV; and SAR-CoV-1, Khosta-2, and BANAL20-247.

In some embodiments of any of the preceding aspects, the antigenic coronavirus peptides include antigenic coronavirus peptides selected from the following combinations of strains:
(i) WA-1, Beta, and Omicron BQ.1.1 (or XBB.1.5), wherein the antigenic coronavirus peptides are RBD antigens comprised in RFN fusion proteins R(WA-1)FN, R(Beta)FN, and R(BQ1.1)FN, or are Spike antigens comprised in S-2P (e.g., S(WA-1)-2P, S(Beta)-2P, and S(BQ1.1)-2P) or SpFN fusion proteins Sp(WA-1)FN, Sp(Beta)FN, and Sp(BQ1.1)FN, or are mosaic antigens comprised in RmosSpFN or RRmosSpFN fusion proteins, or any combination thereof;
(ii) WA-1, Omicron BQ.1.1 (or XBB1.5), and SARS-CoV-1, wherein the antigenic coronavirus peptides are RBD antigens comprised in RFN fusion proteins R(WA-1)FN, R(BQ 1.1)FN, and R(SARS-CoV-1)FN, or are Spike antigens comprised in S-2P (e.g., S(WA-1)-2P, S(BQ 1.1)-2P, and S(SARS-CoV-1)-2P) or SpFN fusion proteins Sp(WA-1)FN, Sp(BQ 1.1)FN, and Sp(SARS-CoV-1)FN, or are mosaic antigens comprised in RmosSpFN or RRmosSpFN fusion proteins, or any combination thereof;
(iii) WA-1, SARS-CoV-1, and Khosta2, wherein the antigenic coronavirus peptides are RBD antigens comprised in RFN fusion proteins R(WA-1)FN, R(SARS-CoV-1)FN, and R(Khosta2)FN, or are Spike antigens comprised in S-2P (e.g., S(WA-1)-2P, S(SARS-CoV-1)-2P, and S(Khosta2)-2P) or SpFN fusion proteins Sp(WA-1)FN, Sp(SARS-CoV-1)FN, and Sp(Khosta2)FN, or are mosaic antigens comprised in RmosSpFN or RRmosSpFN fusion proteins, or any combination thereof;
(iv) WA-1, SARS-CoV-1, and BANAL20-247, wherein the antigenic coronavirus peptides are RBD antigens comprised in RFN fusion proteins R(WA-1)FN, R(SARS-CoV-1)FN, and R(BANAL20-247)FN, or are Spike antigens comprised in S-2P (e.g., S(WA-1)-2P, S(SARS-CoV-1)-2P, and S(BANAL20-247)-2P) or SpFN fusion proteins Sp(WA-1)FN, Sp(SARS-CoV-1)FN, and Sp(BANAL20-247)FN, or are mosaic antigens comprised in RmosSpFN or RRmosSpFN fusion proteins, or any combination thereof;
(v) WA-1, SARS-CoV-1, and MERS-CoV, wherein the antigenic coronavirus peptides are RBD antigens comprised in RFN fusion proteins R(WA-1)FN, R(SARS-CoV-1)FN, and R(MERS-CoV)FN, or are Spike antigens comprised in S-2P (e.g., S(WA-1)-2P, S(SARS-CoV-1)-2P, and S(MERS-CoV)-2P) or SpFN fusion proteins Sp(WA-1)FN, Sp(SARS-CoV-1)FN, and Sp(MERS-CoV)FN, or are mosaic antigens comprised in RmosSpFN or RRmosSpFN fusion proteins, or any combination thereof;
(vi) SAR-CoV-1, Khosta-2, and BANAL20-247, wherein the antigenic coronavirus peptides are RBD antigens comprised in RFN fusion proteins R(SARS-CoV-1)FN, R(Khosta2)FN, and R(BANAL20-247)FN, or are Spike antigens comprised in S-2P (e.g., S(SARS-CoV-1)-2P, S(Khosta2)-2P, and S(BANAL20-247)-2P) or SpFN fusion proteins Sp(SARS-CoV-1)FN, Sp(Khosta2)FN, and Sp(BANAL20-247)FN, or are mosaic antigens comprised in RmosSpFN or RRmosSpFN fusion proteins, or any combination thereof;
(vii) Beta, Omicron BQ.1.1 (or XBB1.5), and SARS-CoV-1, wherein the antigenic coronavirus peptides are RBD antigens comprised in RFN fusion proteins R(Beta)FN, R(BQ 1.1)FN, and R(SARS-CoV-1)FN, or are Spike antigens comprised in S-2P (e.g., S(Beta)-2P, S(BQ 1.1)-2P, and S(SARS-CoV-1)-2P) or SpFN fusion proteins Sp(Beta)FN, Sp(BQ 1.1)FN, and Sp(SARS-CoV-1)FN, or are mosaic antigens comprised in RmosSpFN or RRmosSpFN fusion proteins, or any combination thereof;
(viii) Beta, Omicron XBB.1.5, and SARS-CoV-1, wherein the antigenic coronavirus peptides are RBD antigens comprised in RFN fusion proteins R(Beta)FN, R(XBB 1.5)FN, and R(SARS-CoV-1)FN, or are Spike antigens comprised in S-2P (e.g., S(Beta)-2P, S(XBB 1.5)-2P, and S(SARS-CoV-1)-2P) or SpFN fusion proteins Sp(Beta)FN, Sp(XBB1.5)FN, and Sp(SARS-CoV-1)FN, or are mosaic antigens comprised in RmosSpFN or RRmosSpFN fusion proteins, or any combination thereof;
(ix) Omicron BQ.1.1, SARS-CoV-1, and Khosta2, wherein the antigenic coronavirus peptides are RBD antigens comprised in RFN fusion proteins R(Omicron BQ.1.1)FN, R(SARS-CoV-1)FN, and R(Khosta2)FN, or are Spike antigens comprised in S-2P (e.g., S(Omicron BQ.1.1)-2P, S(SARS-CoV-1)-2P, and S(Khosta2)-2P) or SpFN fusion proteins Sp(Omicron BQ.1.1)FN, Sp(SARS-CoV-1)FN, and Sp(Khosta2)FN, or are mosaic antigens comprised in RmosSpFN or RRmosSpFN fusion proteins, or any combination thereof ***

In some embodiments of any of the preceding aspects, wherein the nanoparticle-forming peptide comprises or is a ferritin protein or a fragment or variant thereof. In some embodiments, the nanoparticle-forming peptide comprises or is Helicobacter pylori ferritin (Hpf) or a fragment or variant thereof. In some embodiments, the nanoparticle-forming peptide comprises an amino acid sequence selected from: ESQVRQQFSKDIEKLLNEQVNKEMQSSNLYMSMSSWCYTHSLDGAGLFLFDHAAEEYE HAKKLIIFLNENNVPVQLTSISAPEHKFEGLTQIFQKAYEHEQHISESINNIVDHAIKSKDH ATFNFLQWYVAEQHEEEVLFKDILDKIELIGNENHGLYLADQYVKGIAKSRKSGS (SEQ ID NO: 1) or a fragment or variant thereof, DIIKLLNEQVNKEMQSSNLYMSMSSWCYTHSLDGAGLFLFDHAAEEYEHAKKLIIFLNE NNVPVQLTSISAPEHKFEGLTQIFQKAYEHEQHISESINNIVDHAIKSKDHATFNFLQWYV AEQHEEEVLFKDILDKIELIGNENHGLYLADQYVKGIAKSRKSGS (SEQ ID NO: 2) or a fragment or variant thereof, and SKDIIKLLNEQVNKEMQSSNLYMSMSSWCYTHSLDGAGLFLFDHAAEEYEHAKKLIIFL NENNVPVQLTSISAPEHKFEGLTQIFQKAYEHEQHISESINNIVDHAIKSKDHATFNFLQW YVAEQHEEEVLFKDILDKIELIGNENHGLYLADQYVKGIAKSRKSGS (SEQ ID NO: 3) or a fragment or variant thereof.

In another aspect, the present disclosure provides a nanoparticle comprising a fusion protein comprising a nanoparticle-forming peptide and at least two antigenic coronavirus peptides selected from: a receptor-binding domain (RBD) of a coronavirus, or a fragment or variant thereof, an N-terminal domain (NTD) of a coronavirus, or a fragment or variant thereof, an S1 domain of a coronavirus, or a fragment or variant thereof, a stabilized extracellular spike S-2P domain of a coronavirus, or a fragment or variant thereof, a stabilized extracellular spike S domain of a coronavirus, or a fragment or variant thereof, a stabilized extracellular spike S-trimer of a coronavirus, or a fragment or variant thereof, and a mosaic coronavirus spike protein, wherein at least one domain of the mosaic coronavirus spike protein is substituted or added from a heterologous coronavirus strain or species.

In some embodiments, the nanoparticle-forming peptide comprises or is a ferritin protein or a fragment or variant thereof. In some embodiments, the nanoparticle-forming peptide comprises or is Helicobacter pylori ferritin (Hpf) or a fragment or variant thereof. In some embodiments, the nanoparticle-forming peptide comprises an amino acid sequence selected from: ESQVRQQFSKDIEKLLNEQVNKEMQSSNLYMSMSSWCYTHSLDGAGLFLFDHAAEEYE HAKKLIIFLNENNVPVQLTSISAPEHKFEGLTQIFQKAYEHEQHISESINNIVDHAIKSKDH ATFNFLQWYVAEQHEEEVLFKDILDKIELIGNENHGLYLADQYVKGIAKSRKSGS (SEQ ID NO:1) or a fragment or variant thereof, DIIKLLNEQVNKEMQSSNLYMSMSSWCYTHSLDGAGLFLFDHAAEEYEHAKKLIIFLNE NNVPVQLTSISAPEHKFEGLTQIFQKAYEHEQHISESINNIVDHAIKSKDHATFNFLQWYV AEQHEEEVLFKDILDKIELIGNENHGLYLADQYVKGIAKSRKSGS (SEQ ID NO:2) or a fragment or variant thereof, and SKDIIKLLNEQVNKEMQSSNLYMSMSSWCYTHSLDGAGLFLFDHAAEEYEHAKKLIIFL NENNVPVQLTSISAPEHKFEGLTQIFQKAYEHEQHISESINNIVDHAIKSKDHATFNFLQW YVAEQHEEEVLFKDILDKIELIGNENHGLYLADQYVKGIAKSRKSGS (SEQ ID NO:3) or a fragment or variant thereof.

In some embodiments of any of the foregoing aspects, at least two of said antigenic coronavirus peptides are comprised in a fusion protein, connected via a linker. In some embodiments, 2-10 of said antigenic coronavirus peptides are comprised in a fusion protein in series, optionally wherein the antigenic coronavirus peptides are connected via a linker. In some embodiments an antigenic coronavirus peptide is comprised in a fusion protein with a nanoparticle-forming peptide, wherein the antigenic coronavirus peptide is connected to the nanoparticle-forming peptide via a linker. In any embodiments comprising a linker, the linker may comprise an amino acid sequence selected from: GGGSGGSG (SEQ ID NO: 583), GSGGGG (SEQ ID NO: 11), GGGG (SEQ ID NO: 15), GSGG (SEQ ID NO: 5), GGG (SEQ ID NO: 16), and SGG (SEQ ID NO: 17).

In some embodiments of any of the foregoing aspects, the first and second antigenic coronavirus peptides are comprised in a mosaic coronavirus spike protein, wherein at least one domain of the mosaic coronavirus spike protein is substituted or added from a heterologous coronavirus strain. In any embodiment wherein an antigenic coronavirus peptide is comprised in a fusion protein with a nanoparticle-forming peptide, the fusion protein may comprise a format selected from SpFN, beads on a string, domain fusion, domain swap, loop insertion, and domain insertion. In some such embodiments, the fusion protein comprises a format selected from the formats shown in FIGS. 2-7. In any such embodiments the first and second antigenic coronavirus peptides may be different RBD peptides from different coronavirus strains (e.g., R₁, R₂) comprised in the same fusion protein (e.g., R₁R₂FN), optionally wherein the fusion protein further comprises a Spike protein (e.g., RiR2mosSpFN), further optionally wherein the composition comprises two or more different fusion proteins comprising the same two or more different RBD peptides in different positions in the fusion protein (e.g., R₁R₂FN, R₂R₁FN or RiR2mosSpFN, R₂R₁mosSpFN), or mRNA molecules encoding said two or more different fusion proteins, or a nanoparticle displaying said two or more different fusion proteins. In any such embodiments, the fusion protein may comprise an amino acid sequence selected from the sequences disclosed in Table 6 and Table 7 (SEQ ID NOs: 29-551), or a sequence having at least 80% sequence identity thereto (e.g., at least 85%, at least 90%, at least 95%, at least 96%, at least 97%, at least 98%, or at least 99%).

In some embodiments, the composition comprises a nanoparticle comprising the at least two antigenic coronavirus peptides

In some embodiments, the composition comprises one or more mRNA molecules encoding the at least two antigenic coronavirus peptides, optionally wherein the one or mRNA molecules are encapsulated or co-encapsulated in one or more lipid nanoparticles (LNPs). In some embodiments, a composition comprises one mRNA molecule encoding one fusion protein comprising the at least two antigenic coronavirus peptides, optionally wherein the fusion protein further comprises a nanoparticle-forming peptide, wherein the mRNA molecule is encapsulated in a lipid nanoparticle (LNP); in some embodiments a composition comprises two or more mRNA molecules, each encoding at least one of the at least two antigenic coronavirus peptides, optionally in a fusion protein with a nanoparticle-forming peptide, wherein each mRNA molecule is encapsulated in a separate lipid nanoparticle (LNP); in some embodiments a composition comprises two or more mRNA molecules, each encoding at least one of the at least two antigenic coronavirus peptides, optionally in a fusion protein with a nanoparticle-forming peptide, wherein two or more mRNA molecules are co-encapsulated in the same lipid nanoparticle (LNP).

In accordance with any of the foregoing aspects or embodiments, when the composition comprises one or more mRNA molecules, the mRNA molecule may have one or more features selected from: a 5′ untranslated region (5′ UTR), a 3′ untranslated region (3′ UTR), a polyadenylation (poly(A)) sequence, a chemical modification, optionally wherein the chemical modification comprises N1-methylpseudouridine, and the mRNA is a self-replicating mRNA or a non-replicating mRNA, optionally wherein the mRNA molecule is encapsulated in a lipid nanoparticle (LNP).

In accordance with any of the foregoing embodiments, the immunogenic composition may further comprise an adjuvant. In some embodiments comprising an antigen or nanoparticle, the adjuvant comprises one or more selected from ALFQ, alhydrogel, and combinations thereof.

In a further aspect, the present disclosure provides nanoparticles comprising a fusion protein comprising a nanoparticle-forming peptide and at least two antigenic coronavirus peptides independently selected from: a receptor-binding domain (RBD) of a coronavirus, or a fragment or variant thereof, an N-terminal domain (NTD) of a coronavirus, or a fragment or variant thereof, an S1 domain of a coronavirus, or a fragment or variant thereof, a stabilized extracellular spike S-2P domain of a coronavirus, or a fragment or variant thereof, a stabilized extracellular spike S domain of a coronavirus, or a fragment or variant thereof, a stabilized extracellular spike S-trimer of a coronavirus, or a fragment or variant thereof, and a mosaic coronavirus spike protein, wherein at least one domain of the mosaic coronavirus spike protein is substituted or added from a heterologous coronavirus strain.

In a further aspect, the present disclosure provides DNA molecules comprising a sequence encoding a nanoparticle disclosed here (e.g., a nanoparticle of the foregoing aspect), or a plasmid comprising said DNA molecule, optionally wherein the plasmid can express the DNA molecule in vivo.

In a further aspect, the present disclosure provides methods of treating or preventing a coronavirus infection in a subject in need thereof, comprising administering to a subject in need thereof an immunogenic composition as disclosed herein (e.g., an immunogenic composition of any of the foregoing aspects or embodiments). Also provided are an immunogenic compositions as described herein for use in treating or preventing a coronavirus infection in a subject in need thereof. Also provided are uses of an immunogenic composition as described herein in the preparation of a medicament for treating or preventing a coronavirus infection in a subject in need thereof. In some embodiments, the subject is at risk of contracting a coronavirus infection.

In some embodiments, the subject has already contracted a coronavirus infection. In some embodiments, the subject has not previously been administered a vaccine for prevention of a coronavirus infection. In some embodiments the subject has previously been administered a vaccine for prevention of a coronavirus infection. In some embodiments, the method, composition for use, or use elicits an immune response in the subject against a coronavirus, optionally wherein the immune response comprises neutralizing antibodies, further optionally wherein the neutralizing antibodies cross-neutralizes two or more coronavirus strains, further optionally wherein the neutralizing antibodies cross-neutralizes one or more coronavirus strains that is not a component strain of the immunogenic composition.

In another aspect, the present disclosure provides an mRNA molecule comprising or consisting of a sequence selected from any one of SEQ ID NOs: 552-582 or a sequence at least 80% homologous thereto. In some embodiments, the mRNA molecule has a sequence comprising or consisting of a sequence with at least 80%, at least 85%, at least 90%, at least 95%, at least 96%, at least 97%, at least 98%, or at least 99% homology or identity to any one of SEQ ID NOs: 552-582. In some embodiments, the mRNA molecule has a sequence comprising any one of SEQ ID NOs: 552-582. In some embodiments, the mRNA molecule has a sequence consisting of any one of SEQ ID NOs: 552-582.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows a schematic of coronavirus phylogenetic tree. Virus species that are known to infect humans and cause significant disease are named and colored light gray e.g., HCoV-229E

FIG. 2 shows linear schematics of pan-CoV nanoparticle design formats. Ten design formats are shown, which include a C-terminal H. pylori ferritin preceded by heterologous receptor-binding domains (RBDs) connected in series, RBD and N-terminal domains (NTDs) in series, RBDs replacing the NTD while retaining the native subdomains (SD) of the S1 polypeptide and RBDs inserted into loops of the NTD. There are three examples of Spike Ferritin Nanoparticles (SpFNs) where heterologous RBD molecules are introduced either to replace the native RBD or NTD domains, or as an additional RBD domain that is connected at the N-terminus. Many of these design formats are orthogonal and can be combined. Specifically, insertions of RBDs into loops of the NTD can be combined with NTD containing constructs. While illustrated with ferritin, ferritin may be replaced by other nanoparticle carrier molecules as discussed herein.

FIG. 3 shows select “beads on a string” formats in graphical representation, with examples provided for (A) RR-FN, (B) RN-FN, (C) RRN-FN, (D) RNRN-FN, (E) R-FN, (F) RR-FN, and (G) RR-FN. For example, one or more RBDs of any combination may be linked in a series to ferritin (“R-FN” or “RR-FN”); one RBD from any coronavirus strain may be linked to an NTD from any coronavirus strain and ferritin (“RN-FN”); two RBDs from different coronavirus strains may be linked in a series to a NTD from a different coronavirus strain and ferritin (“RRN-FN”); and a RBD-NTD-RBD-NTD series, with RBDs or NTDs from any coronavirus strain linked in a series and ferritin (“RNRN-FN”).

FIG. 4 shows select “domain fusion” formats in graphical representation, with examples and domain notation provided for (A) R₂-SD-FN, (B) R-S1-FN, (C) R₂-SD-S2-FN, (D) (R)-R-SpFN, (E) R-SpFN, (F) RR-SpFN, and (G) RR-SpFN.

FIG. 5 shows select “loop insertion” formats in graphical representation, with examples and domain notation provided for a set of chimeric fusion ferritin nanoparticle immunogens where heterologous RBD molecules are inserted into (A) R₂N-FN-70, (B) R₂N-FN-148, and (C) R₂N-FN-164.

FIG. 6 shows select “domain swap” formats in graphical representation (also referred to herein as “mosaic” formats), with examples and domain notation provided for a set of chimeric fusion Spike ferritin nanoparticle immunogens where heterologous RBD molecules are inserted into (A) CoV SpFN molecules (mosaic SpFN) and (B) additional heterologous RBD molecules are added to the N-terminus of the SpFN molecule (mosaic R-mosaic SpFN). The chimeric fusion spike is shown in cartoon representation, with the additional heterologous RBDs indicated.

FIGS. 7A and 7B shows select designs in graphical representation for a set of chimeric fusion Spike Ferritin Nanoparticle (SpFN) immunogens where heterologous RBD or NTD molecules are added to the N-terminus of the mosaic SpFN design (e.g., mosaic formats): FIG. 7A: various R-R-mosaic SpFNs; FIG. 7B: RNR-mosaic SpFN. Additional RBD or NTD molecules can be added to the N-terminus of the mosaic SpFN.

FIG. 8 shows (A) negative-stain electron microscopy of MERS-CoV RBD-ferritin nanoparticles, and (B) 2D-classification of M3 RBD ferritin and M4RBD-Ferritin nanoparticles. Briefly, purified proteins were deposited at 0.02-0.08 mg/ml on carbon-coated copper grids and stained with 0.75% uranyl formate. Grids were imaged using a FEI T20 operating at 200 kV with an Eagle 4K CCD using SerialEM or using a Thermo Scientific Talos L120C operating at 120 kV with Thermo Scientific Ceta using EPU.

FIG. 9 shows a cladogram reflecting phylogenetic grouping of selected sarbecoviruse strains based on sequence similarity of the amino acid sequence of the Spike protein receptor binding domain (RBD).

FIG. 10 shows the results of octet biolayer interferometry binding assays of MERS RBD-ferritin nanoparticle immunogens (M.1-M3.6) assessed for binding to MERS-CoV-neutralizing human monoclonal antibody CDC-C2 in two formats.

FIG. 11 shows negative-stain electron microscopy and binding studies of MERS RBD-ferritin nanoparticle immunogens. Panel A shows SARS2-RBD-MERS-RBD CoV-ferritin constructs form nanoparticles as shown by negative-stain EM, and Panel B shows SARS2-RBD-MERS-RBD CoV-ferritin constructs were assessed for binding to MERS-CoV-neutralizing human monoclonal antibody and SARS-CoV-2-neutralizing mAb. The construct designs for pCoV247 and pCoV248 are shown in Table 3.

FIG. 12 shows, in Panel A, negative-stain electron microscopy of Coronavirus Spike Ferritin nanoparticle constructs form nanoparticles as shown by negative-stain EM; in Panel B, 2D-classification of SARS-1 (SARS-CoV-1), MERS, HKU-1, and 229E spike ferritin nanoparticles, and in Panel C, 3D-reconstruction of HKU-1 and 229E spike ferritin nanoparticles.

FIG. 13 shows a graph reporting results of R-SpFN designs pCoV316 and pCoV317 assessed for binding to neutralizing antibodies ShAb01 (neutralizes SARS-CoV-1 and SARS-CoV-2), ShAb02 (neutralizes SARS-CoV-2), and human ACE2 receptor by biolayer interferometry. Association was allowed to occur for 180 seconds followed by dissociation for 60 seconds.

FIG. 14 shows production and characterization of Construct pCoV323 (RR-SpFN where the RBD from MZ081380_bat_Yunnan_RsYN04_2020, and SARS-1 RBD are linked to the WA-1 SpFN molecule). Left panel: Size-exclusion chromatography of pCoV323 (RR-SpFN) shows the transiently transfected protein forms a large nanoparticle of expected size. Right panel: SDS-PAGE of pCoV323 following purification by NiNTA affinity purification following size-exclusion chromatography.

FIG. 15 shows a cladogram reflecting phylogenetic grouping of selected sarbecoviruse strains based on sequence similarity of the amino acid sequence of the Spike protein receptor binding domain (RBD).

FIG. 16 shows, in Panel A, an illustration of antigenic distance sets. Sarbecovirus strains that are proportionally closer have greater immunological similarity compared to strains that are further distance apart. FIG. 16 also shows, in Panel B, an illustration of a multivalent RFN comprising antigens from three different strains of an antigenic distance set. The different RBDs (i.e., the “R” in “RFN”) are illustrated with different patterns to reflect the different strains of origin.

FIG. 17 shows a summary of antigenic distance sets tested in different antigen presentation formats. (+) indicates constructs expressed from co-encapsulated mRNA molecules; (++) indicates constructs expressed from admixtures of separately encapsulated mRNA molecules; +/−FN indicates formats tested with or without empty ferritin particles.

FIG. 18 shows results from a Western blot following expression of mRNA constructs in HeLa cells (Panel A) and blots showing correct banding (MW) of monovalent constructs (Panel B).

FIG. 19 shows results from Western blot and electron microscopy following expression of mRNA constructs in HeLa cells. Panel A shows the protein from cell lysates; Panel B shows protein from culture supernatants; Panel C shows a negative-stain electron microscope grid of purified SARS-CoV-2 Beta SpFN molecules from the supernatant. The assembly of the particle is indicated by black arrows, the Spike is seen on the surface of the ferritin nanoparticle. Panel D shows the class averages of the SpFN particles, with the Spike and the ferritin particle.

FIG. 20 shows geometric means of pseudoneutralization titers elicited against Coronavirus Clade 1b strains WA-1, Delta, Beta, BA5, BQ.1.1, or XBB.1.5, a SARS-COV-1 strain from Coronavirus Clade 1a, and a Merbicovirus in sera from mice immunized with monovalent RFN (Groups 1-7) (Panel A), SpFN (Groups 18-24) (Panel B), or stabilized transmembrane spike (S2P, Groups 15-17, 37-40) (Panel C). Further details of each group can be found in Table 8.

FIG. 21 shows geometric means of pseudoneutralization titers elicited against Coronavirus Clade 1b strains WA-1, Delta, Beta, BA5, BQ.1.1, or XBB.1.5, a SARS-COV-1 strain from Coronavirus Clade 1a, and a Merbicovirus in sera from mice immunized with multivalent Mixes A-E RFN (groups 8-12) (Panel A), SpFN (Groups 25-29) (Panel B), or stabilized transmembrane spike (S2P, groups 41-44) (Panel C). Further details of each group can be found in Table 8.

FIG. 22 shows geometric means of pseudoneutralization titers elicited against Coronavirus Clade 1b strains (A) WA-1, (B) Delta, (C) Beta, (D) BA5, (E) BQ.1.1, or (F) XBB.1.5 in sera from mice immunized with monovalent RFN or RFN antigenic distance mixes A-E (Groups 1-12). Titers from each group of mice and readout are represented as a box and whisker plot showing the mean (horizontal line), first standard deviation (box), and second standard deviation (vertical line). Any individual titers greater than 2 standard deviations away from the mean (i.e., outliers) are represented as circles. Further details of each group can be found in Table 8.

FIG. 23 shows pseudoneutralization titers elicited against Coronavirus Clade 1b strains (A) WA-1, (B) Delta, (C) Beta, (D) BA5, (E) BQ.1.1, or (F) XBB.1.5 in sera from mice immunized with monovalent SpFN or SpFN antigenic distance mixes A-E (Groups 18-29). Titers from each group of mice and readout are represented as a box and whisker plot showing the mean (horizontal line), first standard deviation (box), and second standard deviation (vertical line). Any individual titers greater than 2 standard deviations away from the mean (i.e., outliers) are represented as circles. Further details of each group can be found in Table 8.

FIG. 24 shows pseudoneutralization titers elicited against Coronavirus Clade 1b strains (A) WA-1, (B) Delta, (C) Beta, (D) BA5, (E) BQ.1.1, or (F) XBB.1.5 in sera from mice immunized with monovalent S2P or S2P antigenic distance mixes A-E (Groups 15-17, 31, 38-44). Titers from each group of mice and readout are represented as a box and whisker plot showing the mean (horizontal line), first standard deviation (box), and second standard deviation (vertical line). Any individual titers greater than 2 standard deviations away from the mean (i.e., outliers) are represented as circles. Further details of each group can be found in Table 8.

FIG. 25 shows geometric mean pseudoneutralization titers elicited against Coronavirus strains WA-1, Delta, Beta, BA5, BQ.1.1, XBB.1.5, SARS1, or MERS in sera from mice immunized with Spike antigens presented as either monovalent or multivalent stabilized transmembrane (S-2P, monovalent groups 15-17, 37-40; multivalent groups 31, 41-44) and monovalent or multivalent spike conjugated to ferritin (SpFN, monovalent groups 18-24; multivalent groups 25-29). Groups were matched based on component strains and plotted in a scatter plot with stabilized transmembrane spike groups forming the x-axis coordinate and the spike conjugated to ferritin groups forming the y-axis coordinate. Monovalent titers (unfilled circles) are distributed around the x=y line (dashed line) suggesting approximately equal titers for each group. Refer to FIGS. 20 and 21 for individual group titers and titer distribution.

FIG. 26 shows pseudoneutralization titers elicited against Coronavirus strains (A) WA-1, (B) Delta, (C) Beta, (D) SARS-1 in sera from mice immunized with Mix C SpFN (Group #), Mix D RFN (Group #), and Mix D RFN with empty ferritin (RFN+FN, Group #). The mRNAs were either administered as a co-administration of an admixture of LNPs separately encapsulating mRNA molecules encoding each construct (admin) or LNPs coencapsulating the mRNA molecules (encap).

FIG. 27 shows pseudoneutralization titers elicited against Coronavirus strain pseudoviruses (A) WA-1, (B) Delta, (C) Beta, (D) SARS-1 by sera from mice immunized with RFN with or without empty ferritin: WA-1 (group 1, 34), SARS-1 (group 4, 36), or BANAL20-247 (group 7, 35). Additionally, multivalent Mix D (WA-1+SARS-1+BANAL20-247) RFN was tested in 4 preparations: each monovalent component (without empty FN) co-administrated (ca −, group 33), each monovalent component co-encapsulated with empty FN co-administered (ca ce +, group 14), co-encapsulated without empty FN (group 13), and co-encapsulated (group 8).

FIG. 28 shows that multivalent particles can be produced by either co-encapsulating multiple mRNAs, each expressing a different fusion protein (illustrated as RFNs of different strains), into a single LNP (as shown in the top panel), or by administering multiple LNPs, each encapsulating a different mRNA.

FIG. 29 shows various mixes of strains used in experiments (Mixes A-E) and the relative antigenic distance of the strains, and schematic diagrams of resulting nanoparticles.

FIG. 30 shows various examples of multivalent particles. The illustrated examples include a particle comprising two different RFN fusion proteins (A) and four different RFN fusion proteins (B), but other particles could be formed from 3, 5, or 6, or more different RFN fusion proteins. The illustrated examples also include a multivalent particle that comprises a single RRFN fusion protein, in which each RBD (i.e., each “R” in the “RRFN”) is from a different strain (C), and a particle comprising two RRFN fusion proteins, wherein the order of the RBD domains is switched between the two different RRFN fusion proteins. Other RRFN particles comprising RRFN fusion proteins with various RBD domains from different stains and in different orders could also be formed.

FIG. 31 shows a dosing regimen for a study of multivalent antigens as described herein as a booster vaccine.

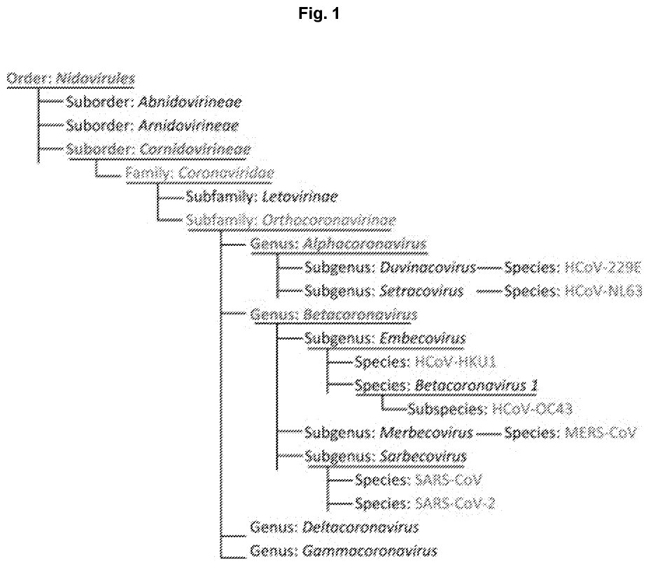

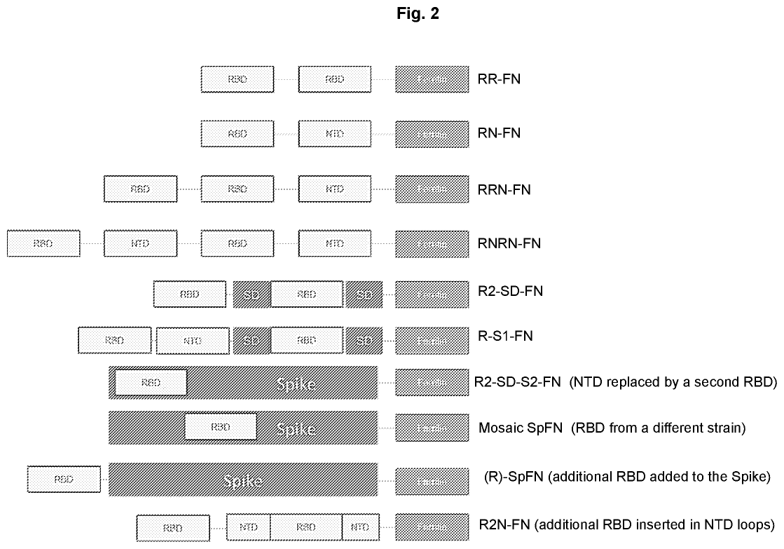

### DETAILED DESCRIPTION

The present disclosure provides immunogenic compositions including nanoparticle vaccines and mRNA molecules encoding them for treating or preventing coronavirus infections and coronavirus infectious diseases, and related infections and diseases caused by sarbecoviruses and merbecoviruses. The disclosed immunogenic compositions comprise at least two antigenic coronavirus peptides comprising at least a first antigenic coronavirus peptide and a second antigenic coronavirus peptide, or one or more messenger RNA (mRNA) molecules encoding them. In accordance with some embodiments, described herein are multivalent immunogenic compositions comprising two or more antigenic coronavirus peptides from different strains, or mRNA molecules encoding them. Heterologous antigens may focus the immune response to create additional breadth of recognized antigens. Further, immunization by multiple heterologous strains, even those across clades, may provide additional breadth of immune response.

The nanoparticles disclosed herein are made up of fusion proteins that comprise a nanoparticle-forming peptide and an antigenic coronavirus peptide (e.g., at least two antigenic coronavirus peptides, optionally from different strains of coronavirus), which may be optionally joined together via a linker. The fusion proteins are capable of self-assembling into nanoparticles that are stable in solution and able to generate a protective neutralizing immune response (i.e., the production of neutralizing antibodies and/or defensive cytokines) when administered to a subject. Likewise, disclosed mRNA molecules, when administered and expressed in vivo, result in the production of antigens that generate a protective neutralizing immune response. In some embodiments, an immunogenic composition comprises one or more mRNA molecules encoding one or more fusion proteins that comprise a nanoparticle-forming peptide and an antigenic coronavirus peptide, and when administered and expressed in vivo, results in the production of a nanoparticle as disclosed herein that generates a protective neutralizing immune response. In any embodiments, an immunogenic composition as disclosed herein may also comprise an adjuvant.

The disclosed immunogenic compositions (e.g., comprising mRNA molecules or nanoparticles) will provide protection against infection by coronaviruses, such as SARS-CoV-2 and other sarbecoviruses, MERS-CoV and other merbecoviruses, and other coronaviruses. The disclosed immunogenic compositions may also reduce illness caused by the coronaviruses. The disclosed immunogenic compositions may elicit protective immune responses in individuals that receive the vaccines.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “can,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the term “comprising” is intended to mean that the compositions and methods include the recited elements, but does not exclude others.

As used herein, a “variant” when used in the context of referring to a peptide means a peptide sequence that is derived from a parent sequence by incorporating one or more amino acid changes, which can include substitutions, deletions, or insertions. For the purposes of this disclosure, a variant may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with the reference (or “parent”) sequence. For purposes of this disclosure, the terms “variant” and “derivative” when used in the context of referring to a peptide are used interchangeably.

As used herein, a “variant” when used in the context of referring to a virus (e.g., SARS-CoV-2) means a virus that is a progeny of a reference (or “parent”) virus that possesses one or more changes in its genome (e.g., a RNA genome), or a virus that is genetically engineered to have one or more changes in its genome, relative to a reference (or “parent”) virus, which may or may not result in changes to the proteins encoded by the RNA sequence (e.g., one or more proteins of a variant virus may include substitutions, deletions, or insertions compared to a parent strain). For example, known variants of SARS-CoV-2 include, but are not limited to, B.1.1.7 (first identified in the United Kingdom), B.1.351 (first identified in South Africa), and P.1 (first identified in Brazil) and Omicron strains including XBB.1.5, EG.5.1, BA.1, BA.2, and BA.5. For the purposes of this disclosure, a variant of a virus may comprise a genome sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with the reference (or “parent”) genome sequence.

As used herein, the phrases “effective amount,” “therapeutically effective amount,” and “therapeutic level” mean the dosage or concentration of a disclosed vaccine that provides the specific pharmacologic effect for which the vaccine is administered in a subject in need of such treatment, i.e. to treat or prevent a coronavirus infection (e.g., MERS, SARS, or COVID-19). It is emphasized that a therapeutically effective amount or therapeutic level of a vaccine will not always be effective in treating or preventing the infections described herein, even though such dosage is deemed to be a therapeutically effective amount by those of skill in the art. For convenience only, exemplary dosages, drug delivery amounts, therapeutically effective amounts, and therapeutic levels are provided herein. The therapeutically effective amount may vary based on the route of administration and dosage form, the age and weight of the subject, and/or the subject's condition, including the type and severity of the coronavirus infection.

The terms “treat,” “treatment” or “treating” as used herein with reference to a coronavirus infection refer to reducing or eliminating viral load or eliminating histopathology or virus presence in the airways or lungs.

The terms “prevent,” “preventing” or “prevention” as used herein with reference to a coronavirus infections refer to precluding or reducing the risk of an infection from developing in a subject exposed to a coronavirus, or to precluding or reducing the risk of developing a high viral load of coronavirus or reducing or eliminating histopathology or virus presence in the airways or lungs. Prevention may also refer to the prevention of a subsequent infection once an initial infection has been treated or cured. Prevention may also refer to the prevention of or reduction of risk of transmission of virus from one subject host to another subject host.

The terms “individual,” “subject,” and “patient” are used interchangeably herein, and refer to any individual mammalian subject, e.g., bovine, canine, feline, equine, or human. In specific embodiments, the subject, individual, or patient is a human.

As used herein, the abbreviation “SD” in the context of the disclosed fusion proteins refers to a subdomain of a coronavirus spike protein. In the spike protein, there is a subdomain 1 and a subdomain 2 (see, e.g., Wrapp et al., Science 367, 1260-1263(2020)). Thus, “SD” could refer to either or both of SD1 and SD2 (i.e., subdomain 1 and 2) of the spike protein.

### II. Coronaviruses

Coronaviruses are a family of viruses (i.e., the Coronaviridae family) that cause respiratory infections in mammals and that comprise a genome that is roughly 30 kilobases in length. The Coronaviridae family is divided into four genera and the genome encodes 28 proteins across multiple open reading frames, including 16 non-structural proteins (nsp) that are post-translationally cleaved from a polyprotein. See, e.g., Letko et al., Nature Microbiology, 2020, 5(4):562-569.

The Coronaviridae family includes both α-coronaviruses or β-coronaviruses, which both mainly infect bats, but can also infect other mammals like humans, camels, and rabbits. β-coronaviruses have, to date, been of greater clinical importance, having caused epidemics of diseases with high mortality such as severe acute respiratory syndrome (SARS-CoV-1), Middle East respiratory syndrome (MERS-CoV), and COVID-19 (SARS-CoV-2). Other disease-causing β-coronaviruses include OC43, and HKU1. Non-limiting examples of disease-causing α-coronaviruses include, but are not limited to, 229E and NL63.

Although SARS-CoV-2 is a newly identified virus, it shares genetic and morphologic features with others in the Coronaviridae family, particularly those from the β-coronavirus genus. The genome of the recently isolated SARS-CoV-2 shares 82% nucleotide identity with human SARS-CoV (SARS-CoV-1) and 89% with bat SARS-like-CoVZXC21 (Lu et al., 2020). The spike (S) glycoprotein, in particular, bears significant structural homology with SARS-CoV-1 compared to other coronaviruses such as MERS-CoV. Like SARS-CoV-1, the surface Spike (S) glycoprotein of SARS-CoV-2 binds the same host receptor, ACE-2, to mediate cell entry (Letko et al., 2020; Yan et al., 2020a). S—a class I fusion protein—is also a critical determinant of viral host range and tissue tropism and the primary target of the host immune response (Li, 2016). As such, most coronavirus vaccine candidates developed to date are based on S or one of its sub-components. Coronavirus S glycoproteins contain three segments: a large ectodomain, a single-pass transmembrane anchor and a short intracellular tail. The ectodomain consists of a receptor-binding subunit, S1, which contains two sub-domains: one at the N-terminus and the other at the C-terminus. The latter comprises the receptor-binding domain (RBD), which serves the vital function of attaching the virus to the host receptor and triggering a conformational change in the protein that results in fusion with the host cell membrane through the S2 subunit. Antibodies have been shown to neutralize viral entry by binding to the RBD of the spike protein. This region is also known to be the most variable part of the protein and is likely responsible for immune escape resulting in re-infection or lowered vaccine efficacy.

As discussed in more detail below, ferritin is a small protein expressed by many organisms that can form into a homotypic 24-mer “nanoparticle.” It has been shown in previous work to serve as an antigen presentation system by decorating the N-terminal region with an antigen of interest. An antigen can be conjugated to the ferritin moiety without detracting from nanoparticle formation. Ferritin with an antigen conjugated via a sufficiently long linker can form quaternary structures such as (8) CoV trimers.

Multiple technology platforms are currently advancing SARS-CoV-2 vaccine development, including nucleic acid vaccines, whole virus vaccines, recombinant protein subunit vaccines and nanoparticle vaccines. Of these vaccine platform types, nanoparticle technologies have previously been shown to improve antigen structure and stability, as well as vaccine targeted delivery, immunogenicity, and safety. Vaccines containing ferritin nanoparticles conjugated to either the spike protein or the RBD region of the spike protein have been demonstrated to evoke an immune response that is protective against subsequent challenge in animal models. Joyce, et al., Science Translational Med., 14 (632) (DOI: 10.1126scitranslmed.abi5735) (Dec. 16, 2021); Joyce et al., Cell Reports, 37: 110143 (Dec. 21, 2021). Antigen display systems with multiple antigens (multivalent) have been demonstrated to increase breadth of responses against coronavirus (see, e.g., Cohen at al., Science, 371: 735-741 (February 2021); Cohen at al., Science 377, eabq0839(2022) (DOI:10.1126/science.abq0839) and influenza (see, e.g., Kanekiyo et al., Nat. Immunol., 20: 367-72 (April 2019). Boosting (or sequential vaccination) with heterologous strains has been shown to improve effectiveness of coverage against heterologous strains from the original strain. See, e.g., Tan et al., N. Eng. J. Med., 385: 1401-06 (August 2021).

In some embodiments of the present disclosure, the coronavirus that is treated or prevented by the disclosed immunogenic compositions (e.g., vaccines) is a β-coronavirus. In some embodiments, the β-coronavirus is selected from the group consisting of severe acute respiratory syndrome coronavirus 2 (SARS-CoV-2) (also known by the provisional name 2019 novel coronavirus, or 2019-nCoV or COVID-19), human coronavirus OC43 (hCoV-OC43), Middle East respiratory syndrome-related coronavirus (MERS-CoV, also known by the provisional name 2012 novel coronavirus, or 2012-nCoV), severe acute respiratory syndrome-related coronavirus (SARS-CoV, also known as SARS-CoV-1), HKU-1, 229E, and NL63. In some embodiments, the 3-coronaviruses is SARS-CoV-2, the causative agent of COVID-19. In some embodiments, the disclosed vaccines may provide a broad spectrum treatment and/or prevention for multiple different types of coronavirus, such as MERS-CoV, SARS-CoV-1, and/or SARS-CoV-2, and/or others.

### III. Immunogenic Compositions

Disclosed herein are immunogenic compositions (e.g., vaccines) that can be used to treat or prevent coronavirus infections. In some aspects, the disclosed immunogenic compositions comprise a fusion protein comprising a nanoparticle-forming peptide and an antigenic coronavirus peptide, which may optionally be connected by a linker (i.e., a “linker domain”). The antigenic coronavirus peptide may comprise one or more fragments or full-length proteins derived from a coronavirus (e.g., SARS-CoV-2 or SARS-CoV-1 or MERS-CoV), as described in more detail below.

### A. Nanoparticle-Forming Peptides

The nanoparticle-forming peptide of an immunogenic composition as disclosed herein may be any suitable nanoparticle-forming peptide. H. pylori ferritin and fragments and variants thereof are particularly suitable to serve as a nanoparticle-forming peptides for vaccines as disclosed herein. Thus, the nanoparticle-forming peptide of a vaccine as disclosed herein may comprise a Helicobacter pylori ferritin protein (HpF) or fragment or variant thereof. For instance, the nanoparticle component may comprise the following amino acid sequence derived from H. pylori ferritin:

(SEQ ID NO: 1)
ESQVRQQFSKDIEKLLNEQVNKEMQSSNLYMSMSSWCYTHSLDGAGLFL
FDHAAEEYEHAKKLIIFLNENNVPVQLTSISAPEHKFEGLTQIFQKAYE
HEQHISESINNIVDHAIKSKDHATFNFLQWYVAEQHEEEVLFKDILDKI
ELIGNENHGLYLADQYVKGIAKSRKSGS.

Thus, the nanoparticle-forming peptide of the vaccine may comprise the foregoing H. pylori ferritin sequence (SEQ ID NO: 1) or a variant thereof, which may comprise 1, 2, 3, 4, 5, 6, 7, 8, 9, 10, 11, 12, 13, 14, or 15 or more substitution, deletion, or insertion mutations. For example, the nanoparticle-forming peptide may comprise a variant of SEQ ID NO: 1 that may comprise a deletion of 1, 2, 3, 4, 5, 6, 7, 8, 9, or 10 or more amino acids from the N-terminal domain of SEQ ID NO: 1. In some embodiments, that nanoparticle-forming peptide may comprise a substitution of the glutamic acid residue (E) at position 13 of SEQ ID NO: 1. In some embodiments, that nanoparticle-forming peptide may comprise a substitution of the glutamic acid residue (E) at position 13 of SEQ ID NO: 1 and a deletion of 1, 2, 3, 4, 5, 6, 7, 8, 9, or 10 or more amino acids from the N-terminal domain of SEQ ID NO: 1, such as in the following sequences:

(SEQ ID NO: 2)
DIIKLLNEQVNKEMQSSNLYMSMSSWCYTHSLDGAGLFLFDHAAEEYEHA
KKLIIFLNENNVPVQLTSISAPEHKFEGLTQIFQKAYEHEQHISESINNI
VDHAIKSKDHATFNFLQWYVAEQHEEEVLFKDILDKIELIGNENHGLYLA
DQYVKGIAKSRKSGS;
or
(SEQ ID NO: 3)
SKDIIKLLNEQVNKEMQSSNLYMSMSSWCYTHSLDGAGLFLFDHAAEEYE
HAKKLIIFLNENNVPVQLTSISAPEHKFEGLTQIFQKAYEHEQHISESIN
NIVDHAIKSKDHATFNFLQWYVAEQHEEEVLFKDILDKIELIGNENHGLY
LADQYVKGIAKSRKSGS.

In some embodiments, the nanoparticle-forming peptide may comprise a variant of any of SEQ ID NO: 1, SEQ ID NO: 2, or SEQ ID NO: 3, which may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with any of SEQ ID NO: 1, SEQ ID NO: 2, or SEQ ID NO: 3.

As noted above, in some embodiments, the nanoparticle-forming peptide may be a non-ferritin-based peptide, such as a peptide that comprises the following sequence or a fragment or variant thereof:

(SEQ ID NO: 4)
MQIYEGKLTAEGLRFGIVASRFNHALVDRLVEGAIDAIVRHGGREEDITL
VRVPGSWEIPVAAGELARKEDIDAVIAIGVLIRGATPHFDYIASEVSKGL
ADLSLELRKPITFGVITADTLEQAIERAGTKHGNKGWEAALSAIEMANLF
KSLR.

In some embodiments, the nanoparticle-forming peptide may comprise a variant of SEQ ID NO: 4, which may comprise 1, 2, 3, 4, 5, 6, 7, 8, 9, 10, 11, 12, 13, 14, or 15 or more substitution, deletion, or insertion mutations in SEQ ID NO: 4. In some embodiments, the nanoparticle-forming peptide may comprise a variant of SEQ ID NO: 4 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 4.

### B. Linker Domains

The disclosed fusion proteins generally comprise a flexible amino acid linker; however, the linker domain (i.e. linker) is optional and in some embodiments the nanoparticle-forming peptide may be directly Joined with the antigenic coronavirus peptide. The linker may be about 3 to about 50 amino acids in length, or more particularly about 4 to about 42 amino acids in length. In some embodiments, the linker may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 amino acids in length. The linker domain may comprise glycine (G) repeats and or a combination of glycine (G) and serine (S) residues. Several exemplary linker sequences are disclosed in Table 1 below.

TABLE 1
Exemplary Linker Sequences

| Linker Sequence | SEQ ID NO |
|---|---|
| GSGG | 5 |
| GSGGSG | 6 |
| GSGGEMKQIEDKIEEILSKIYHIENEIARIKKLIGRG | 7 |
| SGGSG | |
| LDSIKEELDKIHKNGSGGSG | 8 |
| IDSIKEEIDKIHKNGSGGSG | 9 |
| MKQIEDKIEEILSKIYHIENEIARIKKLIGRGSGGSG | 10 |
| GSGGGG | 11 |
| GSG | 12 |
| GSGGSGGSGGSGGG | 13 |
| GGGSGGGSGG | 14 |
| GGGG | 15 |
| GGG | 16 |
| SGG | 17 |
| GGGSGGSG | 583 |

The linker domain may comprise 1, 2, or 3 repeats of any one of SEQ ID NOs: 5-17 or 583. In some embodiments, the linker domain comprises a variant of any one of SEQ ID NOs: 5-17 or 583 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with any one of SEQ ID NOs: 5-17 or 583.

The foregoing linker sequences are not intended to be limiting, and those of skill in the art will understand that other flexible peptide linkers may also be suitable for connecting the nanoparticle-forming peptide and the antigenic coronavirus peptide, based on the guidance provided herein.

### C. Antigenic Coronavirus Peptides

In general, the antigenic coronavirus peptide of the disclosed immunogenic compositions and fusion proteins comprises a coronavirus spike protein (also known as “S protein” or “glycoprotein S”), which is generally responsible for viral entry into a host cell, or a fragment or a variant thereof (such as an RBD domain or a fragment or a variant thereof). In some embodiments, the antigenic coronavirus peptide may comprise 1, 2, or 3 or more distinct domains of a coronavirus spike protein connected together in sequence, and in such embodiments, a linker may optionally separate the distinct domains.

The spike protein is selected as an antigenic coronavirus peptide of vaccines as disclosed herein, because antibodies that develop against this peptide are likely to be neutralizing. The spike protein comprises two functional subunits responsible for binding to the host cell receptor (S₁ subunit) and fusion of the viral and cellular membranes (S2 subunit). A fusion protein of the present disclosure may comprise the entire spike protein, only the S₁ subunit, only the S2 subunit, or any antigenic/immunogenic fragment or variant thereof. In some embodiments, the fusion protein comprises full length coronavirus spike protein sequence. In some embodiments, the fusion protein comprises a variant that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of a coronavirus spike protein (e.g., SEQ ID NO: 18), so long as the fragment is able to elicit an immune response (i.e., it is an antigenic fragment).

While not wanting to be bound by theory, it is understood that the spike protein of SARS-CoV-2 attaches to human angiotensin converting enzyme (ACE)-2 cell surface receptors facilitating human infection. Thus, antibodies that can bind to the spike glycoprotein and prevent interaction with the ACE2 receptor can facilitate protection from infection. The SARS-CoV-2 spike protein (NCBI Reference Sequence: YP_009724390.1) comprises 1273 amino acids and consists of a signal peptide (amino acids 1-13) located at the N-terminus, the S1 subunit (14-685 residues), and the S2 subunit (686-1273 residues); the last two regions are responsible for receptor binding and membrane fusion, respectively. The amino acid sequence is shown below.

(SEQ ID NO: 18)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

Specific domains of the coronavirus spike protein that are particularly useful as an antigenic coronavirus peptide in the context of the present disclosure include:
a receptor-binding domain (RBD) of a coronavirus, or a fragment or variant thereof,
an N-terminal domain (NTD) of a coronavirus, or a fragment or variant thereof,
a receptor-binding domain (RBD)-N-terminal domain chimera of a coronavirus, or a fragment or variant thereof,
an S1 domain of a coronavirus, or a fragment or variant thereof,
a stabilized spike S-2P domain or a stabilized extracellular spike S-2P domain of a coronavirus, or a fragment or variant thereof,
a stabilized spike S domain or a stabilized extracellular spike S domain of a coronavirus, or a fragment or variant thereof, or
a stabilized spike S-trimer or a stabilized extracellular spike S-trimer of a coronavirus, or a fragment or variant thereof.

Thus, an antigenic coronavirus peptide of the present disclosure may comprise an RBD. An RBD may comprise the SARS-CoV-2 RBD amino acid sequence set forth below: NITNLCPFGEVFNATRFASVYAWNRKRISNCVADYSVLYNSASFSTFKCYGVSPTKLND LCFTNVYADSFVIRGDEVRQIAPGQTGKIADYNYKLPDDFTGCVIAWNSNNLDSKVGGN YNYLYRLFRKSNLKPFERDISTEIYQAGSTPCNGVEGFNCYFPLQSYGFQPTNGVGYQPY RVVVLSFELLHAPATVCGP (SEQ ID NO: 19). In some embodiments, the antigenic coronavirus peptide comprises a variant of SEQ ID NO: 19 that may comprise 1, 2, 3, 4, 5, 6, 7, 8, 9, 10, 11, 12, 13, 14, or 15 or more substitution, deletion, or insertion mutations in SEQ ID NO: 19. In some embodiments, the antigenic coronavirus peptide comprises a variant of SEQ ID NO: 19 that may comprise an amino acid sequence that shares about 80%, about 810%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 19. In some embodiments, the antigenic coronavirus peptide comprises a fragment of RBD that may be a fragment of SEQ ID NO: 19 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 19, so long as the fragment is able to elicit an immune response (i.e., it is an antigenic fragment).

The antigenic coronavirus peptide may comprise a variant of an RBD (e.g., SEQ ID NO: 19) with one or more specific modifications made to reduce “sticky” hydrophobic regions, which may increase expression and/or the ability to form nanoparticles, for example, one of more of the following modifications.

TABLE 2
Exemplary Amino Acid Modifications in SARS-CoV-2 RBD

| Mutation | | |
|---|---|---|
| (Location corresponds to | Corresponding | |
| location in full length | mutation location | |
| spike; SEQ ID NO: 18) | in SEQ ID NO: 19 | Location and Concept |
| F456N/K458T | 126/128 | glycan-ACE2BS |
| L455R + Y449K + F490R | 125 + 119 + 160 | ACE2BS |
| L455R | 125 | ACE2BS |
| I468R | 138 | ACE2BS |
| Y453R | 123 | ACE2BS |
| L452R | 122 | ACE2BS |
| L492R | 162 | ACE2BS |
| F490R | 160 | ACE2BS |
| F490A | 160 | ACE2BS |
| 517LLH to 517NKS | 187 | glycan- at the bottom hydrophobic patch |
| L518R | 188 | bottom hydrophobic patch |
| V367T + L335N | 37 + 5 | bottom greasy areas |
| T385N/L387T | 55/57 | glycan-lower hydrophobic patch (not the one |
| | | already covered by a glycan) |
| V382R | 52 | lower hydrophobic patch (not the one already |
| | | covered by a glycan) |
| F377R | 47 | lower hydrophobic patch (not the one already |
| | | covered by a glycan) |
| K417N | 87 | RBD mutation alters antigenicity |
| E484K | 154 | RBD mutation alters antigenicity |
| N501Y | 171 | RBD mutation alters antigenicity |
| K417T/E484K/N501Y | 87/154/171 | Match to variant B.1.351 |

The foregoing modifications may increase the expression and/or nanoparticle formation of fusion proteins comprising an RBD with these modifications.

Additionally or alternatively, the antigenic coronavirus peptide may be or comprise an RBD from a coronavirus other than SARS-CoV-2. For example, the RBD domain may be derived from MERS or SARS-CoV-1 (also referred to herein as SARS1 and SARS-1). Exemplary RBD sequences can be found in the full length constructs provided in attached Table 6 and Table 7. Moreover, in some embodiments, a particle may comprise multiple RBDs from the same or different coronaviruses, such as discussed in more detail below.

Additionally or alternatively, an antigenic coronavirus peptide of the present disclosure may comprise an NTD. An NTD may comprise the SARS-CoV-2 NTD amino acid sequence QCVNLTTRTQLPPAYTNSFTRGVYYPDKVFRSSVLHSTQDLFLPFFSNVTWFHAIHVSGT NGTKRFDNPVLPFNDGVYFASTEKSNIIRGWIFGTTLDSKTQSLLIVNNATNVVIKVCEF QFCNDPFLGVYYHKNNKSWMESEFRVYSSANNCTFEYVSQPFLMDLEGKQGNFKNLRE FVFKNIDGYFKIYSKHTPINLVRDLPQGFSALEPLVDLPIGINITRFQTLLALHRSYLTPGD SSSGWTAGAAAYYVGYLQPRTFLLKYNENGTITDAVDCALDPLSETKCTL (SEQ ID NO: 20). In some embodiments, the antigenic coronavirus peptide comprises a variant of SEQ ID NO: 20 that may comprise 1, 2, 3, 4, 5, 6, 7, 8, 9, 10, 11, 12, 13, 14, or 15 or more substitution, deletion, or insertion mutations in SEQ ID NO: 20. In some embodiments, the antigenic coronavirus peptide comprises a variant of SEQ ID NO: 20 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 20. In some embodiments, the antigenic coronavirus peptide comprises a fragment of NTD that may be a fragment of SEQ ID NO: 20 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 20, so long as the fragment is able to elicit an immune response (i.e., it is an antigenic fragment).

Additionally or alternatively, an antigenic coronavirus peptide of the present disclosure may be or comprise an NTD from a coronavirus other than SARS-CoV-2. For example, the NTD domain may be derived from MERS or SARS-CoV-1. Exemplary NTD sequences can be found in the full length constructs provided in attached Table 6 and Table 7. Moreover, in some embodiments, a particle may comprise multiple NTDs from the same or different coronaviruses.

In some embodiments, a particle may comprise a combination of one or more RBD(s) and one or more NTD(s), and the RBD(s) and NTD(s) may be derived from the same or different coronaviruses or strains.

Additionally or alternatively, an antigenic coronavirus peptide of the present disclosure may comprise an S1 protein sequence. An S1 protein sequence may comprise a SARS-CoV-2 S1 protein amino acid sequence VNLTTRTQLPPAYTNSFTRGVYYPDKVFRSSVLHSTQDLFLPFFSNVTWFHAIHVSGTNG TKRFDNPVLPFNDGVYFASTEKSNIIRGWIFGTTLDSKTQSLLIVNNATNVVIKVCEFQFC NDPFLGVYYHKNNKSWMESEFRVYSSANNCTFEYVSQPFLMDLEGKQGNFKNLREFVF KNIDGYFKIYSKHTPINLVRDLPQGFSALEPLVDLPIGINITRFQTLLALHRSYLTPGDSSS GWTAGAAAYYVGYLQPRTFLLKYNENGTITDAVDCALDPLSETKCTLKSFTVEKGIYQT SNFRVQPTESIVRFPNITNLCPFGEVFNATRFASVYAWNRKRISNCVADYSVLYNSASFS TFKCYGVSPTKLNDLCFTNVYADSFVIRGDEVRQIAPGQTGKIADYNYKLPDDFTGCVI AWNSNNLDSKVGGNYNYLYRLFRKSNLKPFERDISTEIYQAGSTPCNGVEGFNCYFPLQ SYGFQPTNGVGYQPYRVVVLSFELLHAPATVCGPKKSTNLVKNKCVNFNFNGLTGTGV LTESNKKFLPFQQFGRDIADTTDAVRDPQTLEILDITPCSFGGVSVITPGTNTSNQVAVLY QDVNCTEVPVAIHADQLTPTWRVYSTGSNVFQTRAGCLIGAEHVNNSYECDIPIGAGICA SYQTQT (SEQ ID NO: 21) or QCVNLTTRTQLPPAYTNSFTRGVYYPDKVFRSSVLHSTQDLFLPFFSNVTWFHAIHVSGT NGTKRFDNPVLPFNDGVYFASTEKSNIIRGWIFGTTLDSKTQSLLIVNNATNVVIKVCEF QFCNDPFLGVYYHKNNKSWMESEFRVYSSANNCTFEYVSQPFLMDLEGKQGNFKNLRE FVFKNIDGYFKIYSKHTPINLVRDLPQGFSALEPLVDLPIGINITRFQTLLALHRSYLTPGD SSSGWTAGAAAYYVGYLQPRTFLLKYNENGTITDAVDCALDPLSETKCTLKSFTVEKGI YQTSNFRVQPTESIVRFPNITNLCPFGEVFNATRFASVYAWNRKRISNCVADYSVLYNSA SFSTFKCYGVSPTKLNDLCFTNVYADSFVIRGDEVRQIAPGQTGKIADYNYKLPDDFTGC VIAWNSNNLDSKVGGNYNYLYRLFRKSNLKPFERDISTEIYQAGSTPCNGVEGFNCYFP LQSYGFQPTNGVGYQPYRVVVLSFELLHAPATVCGPKKSTNLVKNKCVNFNFNGLTGT GVLTESNKKFLPFQQFGRDIADTTDAVRDPQTLEILDITPCSFGGVSVITPGTNTSNQVAV LYQDVNCTEVPVAIHADQLTPTWRVYSTGSNVFQTRAGCLIGAEHVNNSYECDIPIGAGI CASYQTQTNSPRRAR (SEQ ID NO: 22) or SSQCVNLTTRTQLPPAYTNSFTRGVYYPDKVFRSSVLHSTQDLFLPFFSNVTWFHAIHVS GTNGTKRFDNPVLPFNDGVYFASTEKSNIIRGWIFGTTLDSKTQSLLIVNNATNVVIKVC EFQFCNDPFLGVYYHKNNKSWMESEFRVYSSANNCTFEYVSQPFLMDLEGKQGNFKNL REFVFKNIDGYFKIYSKHTPINLVRDLPQGFSALEPLVDLPIGINITRFQTLLALHRSYLTP GDSSSGWTAGAAAYYVGYLQPRTFLLKYNENGTITDAVDCALDPLSETKCTLKSFTVEK GIYQTSNFRVQPTESIVRFPNITNLCPFGEVFNATRFASVYAWNRKRISNCVADYSVLYN SASFSTFKCYGVSPTKLNDLCFTNVYADSFVIRGDEVRQIAPGQTGKIADYNYKLPDDFT GCVIAWNSNNLDSKVGGNYNYLYRLFRKSNLKPFERDISTEIYQAGSTPCNGVEGFNCY FPLQSYGFQPTNGVGYQPYRVVVLSFELLHAPATVCGPKKSTNLVKNKCVNFNFNGLT GTGVLTESNKKFLPFQQFGRDIADTTDAVRDPQTLEILDITPCSFGGVSVITPGTNTSNQV AVLYQDVNCTEVPVAIHADQLTPTWRVYSTGSNVFQTRAGCLIGAEHVNNSYECDIPIG AGICASYQTGGSQSIIAYT (SEQ ID NO: 23) In some embodiments, the antigenic coronavirus peptide may comprise a variant of SEQ ID NO: 21, SEQ ID NO: 22, or SEQ ID NO: 23 that may comprise 1, 2, 3, 4, 5, 6, 7, 8, 9, 10, 11, 12, 13, 14, or 15 or more substitution, deletion, or insertion mutations in SEQ ID NO: 21, SEQ ID NO: 22, or SEQ ID NO: 23. In some embodiments, the antigenic coronavirus peptide may comprise a variant of SEQ ID NO: 21, SEQ ID NO: 22, or SEQ ID NO: 23 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 21, SEQ ID NO: 22, or SEQ ID NO: 23. In some embodiments, the antigenic coronavirus peptide may comprise a fragment of S1 that may be a fragment of SEQ ID NO: 21, SEQ ID NO: 22, or SEQ ID NO: 23 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 21, SEQ ID NO: 22, or SEQ ID NO: 23, so long as the fragment is able to elicit an immune response (i.e., it is an antigenic fragment).

Additionally or alternatively, an antigenic coronavirus peptide of the present disclosure may comprise an S-2P sequence or a fragment or variant thereof. An S-2P sequence is a stabilized version of the spike ectodomain that includes two proline substitutions and stabilizes the prefusion conformation. Alternatively, the S-2P domain includes a transmembrane domain. Specifically, S-2P comprises proline modifications K986P and V987P, as well as the removal of the Furin cleavage site (RRAS to GSAS). In some embodiments, the antigenic coronavirus peptide may comprise a variant of the S-2P sequence that may comprise 1, 2, 3, 4, 5, 6, 7, 8, 9, 10, 11, 12, 13, 14, or 15 or more substitution, deletion, or insertion mutations in the S-2P sequence. In some embodiments, the antigenic coronavirus peptide may comprise a variant of the S-2P sequence that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with a stabilized S-2P. In some embodiments, the antigenic coronavirus peptide may comprise a fragment of S-2P that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the stabilized S-2P, so long as the fragment is able to elicit an immune response (i.e., it is an antigenic fragment).

Additionally or alternatively, an antigenic coronavirus peptide of the present disclosure may comprise a spike S domain or an extracellular spike S domain (e.g., a stabilized spike S domain or stabilized extracellular spike S domain) or a fragment or variant thereof. A stabilized extracellular spike S domain may comprise one or more modifications to stabilize the refusion conformation of the domain or extracellular domain. In some embodiments, the antigenic coronavirus peptide may comprise a stabilized extracellular spike S domain that comprises 1, 2, 3, 4, 5, 6, 7, 8, 9, 10, 11, 12, 13, 14, or 15 or more substitution, deletion, or insertion mutations in an extracellular spike S domain. In some embodiments, the antigenic coronavirus peptide may comprise a stabilized extracellular spike S domain that comprises an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with an extracellular spike S domain. In some embodiments, the antigenic coronavirus peptide may comprise a fragment of the extracellular spike S domain (e.g., a fragment of a stabilized extracellular spike S domain) that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the extracellular spike S domain (e.g., a stabilized extracellular spike S domain), so long as the fragment is able to elicit an immune response (i.e., it is an antigenic fragment). In some embodiments, the antigenic coronavirus peptide may comprise a stabilized spike S domain that comprises 1, 2, 3, 4, 5, 6, 7, 8, 9, 10, 11, 12, 13, 14, or 15 or more substitution, deletion, or insertion mutations in a spike S domain. In some embodiments, the antigenic coronavirus peptide may comprise a stabilized spike S domain that comprises an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with a spike S domain. In some embodiments, the antigenic coronavirus peptide may comprise a fragment of the spike S domain (e.g., a fragment of a stabilized spike S domain) that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the spike S domain (e.g., a stabilized spike S domain), so long as the fragment is able to elicit an immune response (i.e., it is an antigenic fragment).

Additionally or alternatively, an antigenic coronavirus peptide as described herein may comprise a spike S trimer or an extracellular spike S trimer (e.g., a stabilized spike S trimer or stabilized extracellular spike S trimer) or a fragment or variant thereof. A stabilized extracellular spike S trimer may comprise one or more modifications to stabilize the refusion conformation of the extracellular trimer. In some embodiments, the antigenic coronavirus peptide may comprise a stabilized extracellular spike S trimer that comprises 1, 2, 3, 4, 5, 6, 7, 8, 9, 10, 11, 12, 13, 14, or 15 or more substitution, deletion, or insertion mutations in an extracellular spike S trimer. In some embodiments, the antigenic coronavirus peptide may comprise a stabilized extracellular spike S trimer that comprises an amino acid sequence that shares about 80%, about 810%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with an extracellular spike S trimer. In some embodiments, the antigenic coronavirus peptide may comprise a fragment of the extracellular spike S trimer (e.g., a fragment of a stabilized extracellular spike S trimer) that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of extracellular spike S trimer (e.g., a stabilized extracellular spike S trimer), so long as the fragment is able to elicit an immune response (i.e., it is an antigenic fragment). A stabilized spike S trimer may comprise one or more modifications to stabilize the prefusion conformation of the trimer (e.g., a trimerization domain). In some embodiments, the antigenic coronavirus peptide may comprise a stabilized spike S trimer that comprises 1, 2, 3, 4, 5, 6, 7, 8, 9, 10, 11, 12, 13, 14, or 15 or more substitution, deletion, or insertion mutations in a spike S trimer. In some embodiments, the antigenic coronavirus peptide may comprise a stabilized spike S trimer that comprises an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with a spike S trimer. In some embodiments, the antigenic coronavirus peptide may comprise a fragment of the spike S trimer (e.g., a fragment of a stabilized extracellular spike S trimer) that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the spike S trimer (e.g., a stabilized spike S trimer), so long as the fragment is able to elicit an immune response (i.e., it is an antigenic fragment).

Additionally or alternatively, an antigenic coronavirus peptide as described herein may comprise a stabilized variant with six prolines (i.e., “Hexapro”), which is another variant of the spike protein that comprises F817P, A892P, A899P, and A942P substitutions in addition to the two proline substitutions of S-2P. In some embodiments, the antigenic coronavirus peptide may comprise a variant of Hexapro that may comprise 1, 2, 3, 4, 5, 6, 7, 8, 9, 10, 11, 12, 13, 14, or 15 or more substitution, deletion, or insertion mutations in a Hexapro. In some embodiments, the antigenic coronavirus peptide may comprise a variant of Hexapro that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with a Hexapro. In some embodiments, the antigenic coronavirus peptide may comprise a fragment of Hexapro that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the Hexapro, so long as the fragment is able to elicit an immune response (i.e., it is an antigenic fragment).

Additionally or alternatively, an antigenic coronavirus peptide as described herein may comprise a SARS-CoV-1 spike protein (S protein) or a fragment or variant thereof. The SARS-CoV-1 spike protein may comprise the amino acid sequence set forth below: SDLDRCTTFDDVQAPNYTQHTSSMRGVYYPDEIFRSDTLYLTQDLFLPFYSNVTGFHTIN HTFGNPVIPFKDGIYFAATEKSNVVRGWVFGSTMNNKSQSVIIINNSTNVVIRACNFELC DNPFFAVSKPMGTQTHTMIFDNAFNCTFEYISDAFSLDVSEKSGNFKHLREFVFKNKDGF LYVYKGYQPIDVVRDLPSGFNTLKPIFKLPLGINITNFRAILTAFSPAQDIWGTSAAAYFV GYLKPTTFMLKYDENGTITDAVDCSQNPLAELKCSVKSFEIDKGIYQTSNFRVVPSGDV VRFPNITNLCPFGEVFNATKFPSVYAWERKKISNCVADYSVLYNSTFFSTFKCYGVSATK LNDLCFSNVYADSFVVKGDDVRQIAPGQTGVIADYNYKLPDDFMGCVLAWNTRNIDAT STGNYNYKYRYLRHGKLRPFERDISNVPFSPDGKPCTPPALNCYWPLNDYGFYTTTGIG YQPYRVVVLSFELLNAPATVCGPKLSTDLIKNQCVNFNFNGLTGTGVLTPSSKRFQPFQQ FGRDVSDFTDSVRDPKTSEILDISPCAFGGVSVITPGTNASSEVAVLYQDVNCTDVSTAIH ADQLTPAWRIYSTGNNVFQTQAGCLIGAEHVDTSYECDIPIGAGICASYHTVSLLRSTSQ KSIVAYTMSLGADSSIAYSNNTIAIPTNFSISITTEVMPVSMAKTSVDCNMYICGDSTECA NLLLQYGSFCTQLNRALSGIAAEQDRNTREVFAQVKQMYKTPTLKYFGGFNFSQILPDP LKPTKRSFIEDLLFNKVTLADAGFMKQYGECLGDINARDLICAQKFNGLTVLPPLLTDD MIAAYTAALVSGTATAGWTFGAGAALQIPFAMQMAYRFNGIGVTQNVLYENQKQIAN QFNKAISQIQESLTTTSTALGKLQDVVNQNAQALNTLVKQLSSNFGAISSVLNDILSRLDP PEAEVQIDRLITGRLQSLQTYVTQQLIRAAEIRASANLAATKMSECVLGQSKRVDFCGKG YHLMSFPQAAPHGVVFLHVTYVPSQERNFTTAPAICHEGKAYFPREGVFVFNGTSWFIT QRNFFSPQIITTDNTFVSGNCDVVIGIINNTVYDPLQSELDSIKEELDKIHKN (SEQ ID NO: 24). In some embodiments, the antigenic coronavirus peptide comprises a variant of SEQ ID NO: 24 that may comprise 1, 2, 3, 4, 5, 6, 7, 8, 9, 10, 11, 12, 13, 14, or 15 or more substitution, deletion, or insertion mutations in SEQ ID NO: 24. In some embodiments, the antigenic coronavirus peptide comprises a variant of SEQ ID NO: 24 that may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to 100% sequence identity or homology with SEQ ID NO: 24. In some embodiments, the antigenic coronavirus peptide comprises a fragment of a SARS-CoV-1 spike protein that may be a fragment of SEQ ID NO: 24 that comprises about 20%, about 25%, about 30%, about 35%, about 40%, about 45%, about 50%, about 55%, about 60%, about 65%, about 70%, about 75%, about 80%, about 85%, about 90%, 91%, 92%, 93%, 94%, 95%, 96%, 97%, 98%, or 99% of the length of SEQ ID NO: 24, so long as the fragment is able to elicit an immune response (i.e., it is an antigenic fragment).

### D. Fusion Proteins and Vaccine Nanoparticles

The disclosed vaccine nanoparticles are made up of a plurality of fusion glycoprotein domains that self-assemble into a nanoparticle. As noted above, the fusion proteins comprise a nanoparticle-forming peptide, which may be an H. pylori ferritin protein or a fragment or variant thereof. Ferritin is a naturally occurring protein that self-assembles into a 24-member spherical particle, made up of multiple three-fold, four-fold, and/or two-fold axes. Thus, the nanoparticle may comprise a 3-fold axis, a 4-fold axis, or a 2-fold axis. With a 3-fold axes, 8 antigenic trimeric coronavirus peptides can be presented on the surface of the self-assembling protein nanoparticle surface. In the case of monomeric antigens such as RBD, 24 coronavirus peptides can be presented on the surface of the self-assembling protein nanoparticle surface.

### E. Multivalent Immunogenic Compositions

As noted above, the evolution of variants of SARS-CoV-2 has presented a challenge to vaccine efficacy. As a virus evolves, the amino acids within an epitope of a protein antigen may mutate. Due to this process, the antibodies generated by a host to one protein (e.g., after exposure to the virus or immunization with the protein) may bind strongly, bind weakly, or not bind at all to the same epitope of an evolutionarily related protein. If binding is observed at a similar location in two or more related (but not identical) strains, the binding location may be referred to as a “common” epitope, which typically is a neutralizing epitope. As neutralizing epitopes, common epitopes may be subject to evolutionary pressure and may exhibit short-term evolution (e.g., changes in amino acid sequence). By definition, although a common epitope has the potential to mutate, it has thus far not mutated enough to extinguish binding by an overlapping population of antibodies, and so remains common to the strains. Due to the variability of common epitopes, binding is unlikely to be observed in more distantly related strains. Generating antibodies that are more tolerant to variability in common epitopes (e.g., that exhibit binding across common epitopes) represents one approach to generating a broad antigenic response that could offer protection against current and future circulating coronavirus strains. Less commonly recognized epitopes (i.e., sub-dominant epitopes) may not change on a small time-scale (i.e. season-to-season) due to lack of evolutionary pressure, but still can change on a larger time-scale (i.e. decades/centuries) due to evolutionary drift. In some cases, mutations may be less tolerated for sub-dominant epitopes that coincide with functional regions of the protein. If antibodies consistently bind one such an epitope in distantly related antigens, that binding location may be referred to as a “conserved” epitope. Generating antibodies to less commonly recognized conserved epitopes represents another approach to generating a broad antigenic response that could offer protection against current and future circulating coronavirus strains. The multivalent embodiments described herein may leverage one or both of these approaches, by inducing antibodies to common and/or conserved epitopes.

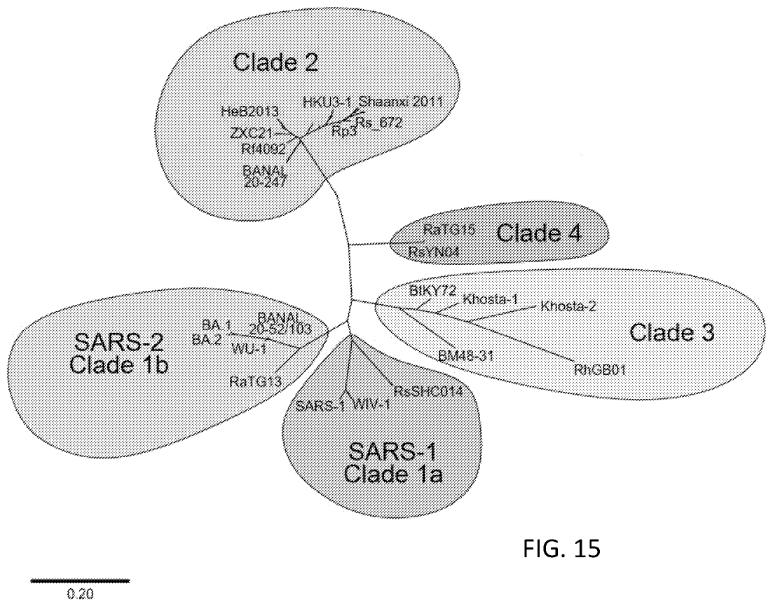

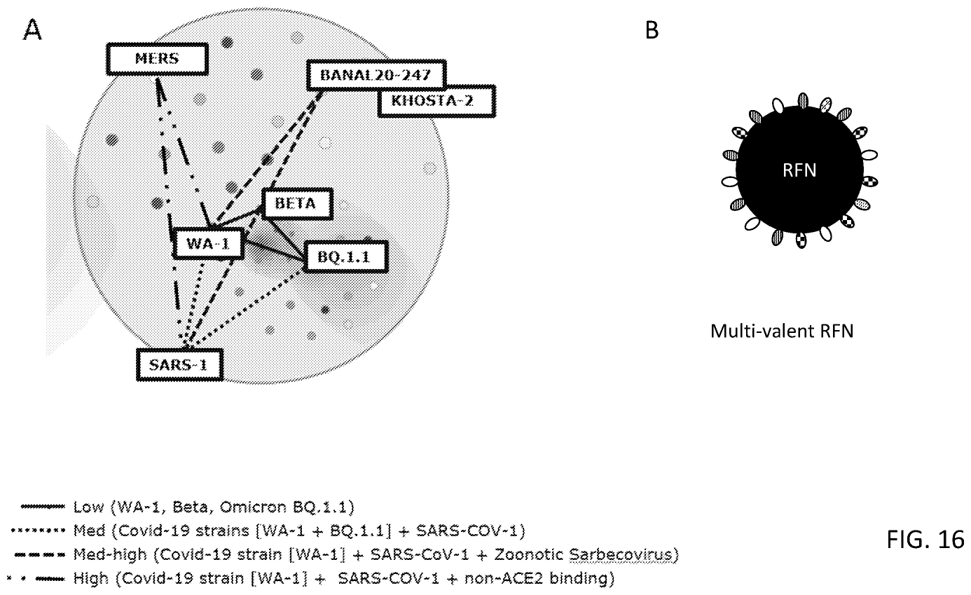

In some aspects, the present disclosure relates to optimized multivalent presentations of antigenic coronavirus peptide on nanoparticles (e.g., on mRNA-encoded ferritin nanoparticles) that, when administered, generate a neutralizing immune response to a broad set of coronaviruses (FIG. 15). As described herein, the antigenic breadth of the immune response is enhanced through the simultaneous, localized presentation of multiple antigens to the immune system. This multivalent vaccination may be achieved via mosaic antigens, such as may be formed in vivo after administration of nanoparticles or mRNA molecules encoding fusion proteins comprising one or more antigenic coronavirus peptides and a nanoparticle-forming protein such as ferritin. mRNA molecules encoding different fusion proteins (containing different antigenic coronavirus peptides) may be co-encapsulated in one lipid nanoparticle (LNP) or encapsulated in separate LNPs formulated together in an immunogenic composition to achieve simultaneous delivery. Once administered, in vivo expressions of the mRNA molecule(s) results in the formation of multivalent nanoparticles displaying the antigenic coronavirus peptides from different strains. These antigens may be from close, distant or diverse sarbecovirus strains. (FIG. 16, Panel A). The co-localized presentation of multiple antigens from strains having selected antigenic distance (as illustrated in FIG. 16, Panel B) is designed to promote maturation of antibodies able to bind epitopes from multiple antigens from diverse coronavirus strains. This will likely increase the breadth of protection against current and future circulating coronavirus strains.

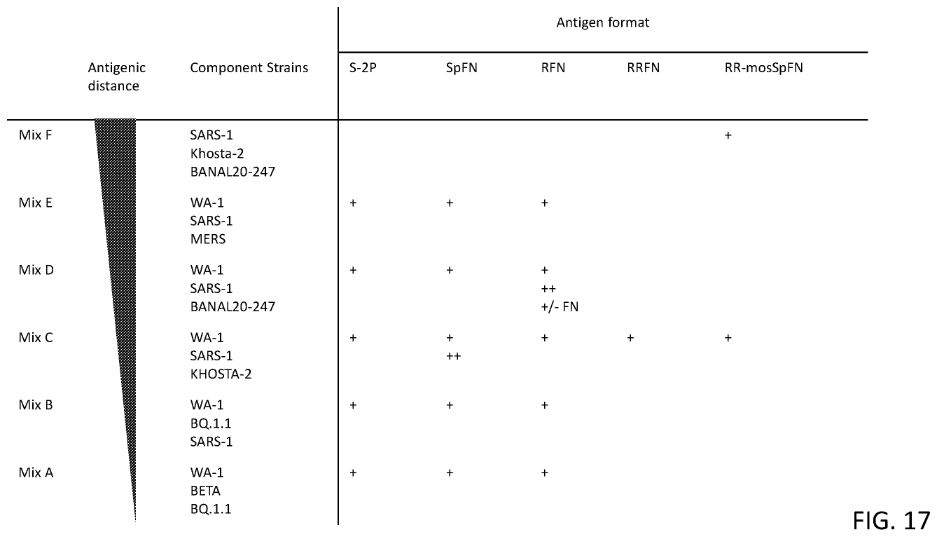

Thus, some aspects of the present disclosure relate to the co-localized presentation of multiple antigens from different strains having a selected antigenic distance (as illustrated in FIG. 17). In this context, antigenic distance refers to the immunogenic similarity of the antibody binding profile of sera generated by exposure to two different antigens, as measured by their similarity in readouts in an assay (e.g., a pseudoneutralization assay). Smaller antigenic distances imply more immunogenic similarity (e.g., higher similarity of neutralization titers across multiple strains) while larger distances imply immunogenic difference (e.g., lower similarity of neutralization titers across multiple strains). Biologically, antigenic similarity refers to sera generated by two different antigens having antibodies that bind to overlapping and/or the same epitopes. Thus, antigens with small antigenic distance are expected to have common epitopes that share physiochemical characteristics to promote binding of similar antibodies, while those at further distance might lack common epitopes, promoting binding of non-overlapping sets of antibodies. In some cases, an antigen may induce a response against a distantly related strain; this is likely mediated by an antibody that binds to a sub-dominant conserved epitope.

In accordance with some multivalent embodiments, the present disclosure provides immunogenic compositions designed based on antigens of antigenic distance sets that, when presented in close physical proximity, promote the generation of broadly binding antibodies. By presenting antigens in close physical proximity, such as on the same nanoparticle, the immune system will likely elicit antibodies that bind two different but evolutionarily related epitopes simultaneously. This provides an additional breadth of immune response because an antibody that binds two variable common epitopes would bind a wider breadth of antigens. The immune system also could elicit antibodies that bind two sub-dominant conserved epitopes, such as due to tolerance of mutations in those two epitopes and/or stronger binding by the antigen binding sites of the antibody leading to stabilization of the antibody-antigen interaction. Thus, as noted above, the multivalent embodiments described herein induce antibodies to common and/or conserved epitopes. FIG. 15 shows how coronaviruses are grouped phylogenically into clades according to sequence similarity in the RBD protein. FIG. 16, Panel A, provides an antigen distance map for several strains. FIG. 17 illustrates various multivalent embodiments (Mix A-Mix F) based on antigenic distance sets.

The antigenic coronavirus peptide components of the disclosed immunogenic compositions or fusion proteins comprised therein or mRNA encoding the same may comprise 1, 2, or 3, or more distinct domains or parts, which may be selected from the exemplary antigenic peptides discussed above. For example, in some embodiments the antigenic coronavirus peptide(s) may comprise one or more domains selected from a RBD, a NTD, a full spike protein, a stabilized extracellular spike S-2P domain, a stabilized extracellular spike S domain, a stabilized extracellular spike S-trimer (optionally with a transmembrane component), and variants or fragments thereof. For example, the antigenic coronavirus peptide(s) of an immunogenic composition as disclosed herein may comprise a combination of two domains, such as two domains independently selected from a RBD, a NTD, a full spike protein, a stabilized extracellular spike S-2P domain, a stabilized extracellular spike S domain, a stabilized extracellular spike S-trimer, a HexaPro, and variants or fragments thereof. Alternatively, the antigenic coronavirus peptides may comprise a combination of three domains, such as three domains independently selected from a RBD, a NTD, a full spike protein, an S1 subunit, an S2 subunit, a stabilized extracellular spike S-2P domain, a stabilized extracellular spike S domain, a stabilized extracellular spike S-trimer, a HexaPro, and variants or fragments thereof. For non-nanoparticle embodiments, the antigenic coronavirus peptide(s) may additionally or alternatively comprise one or more domains selected from a stabilized spike S-2P domain, a stabilized spike S domain, a stabilized spike S-trimer, and variants or fragments thereof. Because the disclosed immunogenic compositions (e.g., vaccines) may be capable of eliciting an immune response from multiple types of coronaviruses (i.e., SARS, MERS, etc.), the various domains of the antigenic coronavirus peptide(s) may be derived from different coronaviruses, different strains of the same coronavirus, or combinations thereof, as noted above and illustrated in more detail below.

Exemplary fusion protein formats include, but are not limited to, a fusion protein comprising (1) a RBD and ferritin (“R-FN”), (2) a spike protein from a strain of coronavirus and ferritin (“SpFN”), optionally wherein the spike protein has an RBD from a different strain (“mosaic SpFN”), (3) a RBD-RBD and ferritin, wherein the two RBDs can be from two different strains of coronavirus (“RR-FN”), (4) a RBD-NTD and ferritin (“RN-FN”), (5) a RBD-RBD-NTD and ferritin (“RRN-FN”), (6) RBD-NTD-RBD-NTD and ferritin “RNRN-FN”), (7) a RBD-SD-RBD-SD (“R2-SD-FN”) and ferritin, (8) a RBD-NTD-SD-RBD-SD and ferritin (“R-S1-FN”), (9) a spike protein in which NTD has been replaced by a second RBD and ferritin (“R2-SD-S2-FN”), (10) a RBD-spike protein in which the RBD is from a different strain of coronavirus and ferritin (“RmosSpFN”), (11) a RBD-RBD-spike protein in which the RBDs are from different strains of coronavirus and ferritin (“RR-mos-SpFN”), (12) a spike protein with an additional RBD and ferritin (“(R)-SpFN”), (13) a spike protein with two additional RBDs and ferritin, wherein the two RBDs can be from two different strains of coronavirus (“RR-SpFN”), and (14) a RBD-NTD/RBD/NTD, in which the second RBD is inserted in the NTD loops, and ferritin (“R2N-FN”).

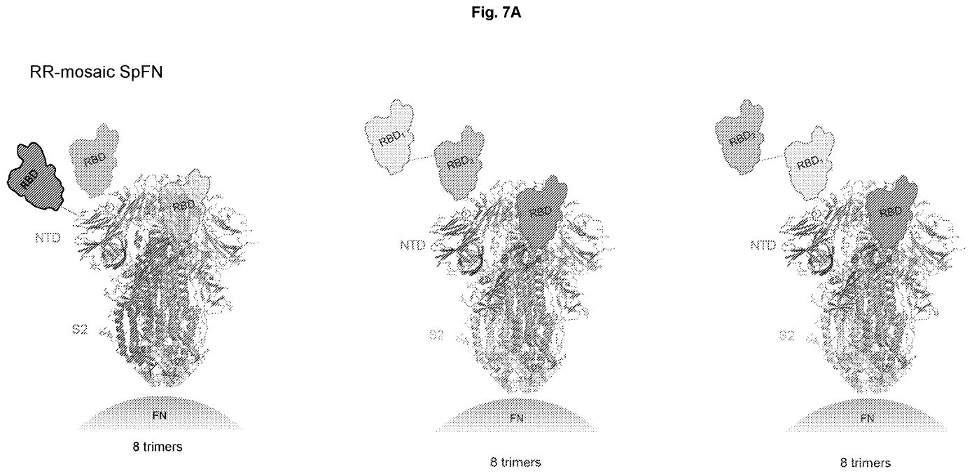

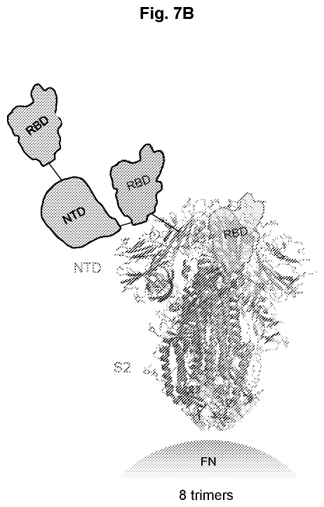

Linear diagrams of many of these fusion protein constructs are shown in FIG. 2-FIG. 7. The various combinations of RBDs, NTDs, and SDs utilized in the disclosed fusion proteins can be from the same strain of coronavirus or different strains of coronavirus, but in multivalent embodiments are from at least two different strains.

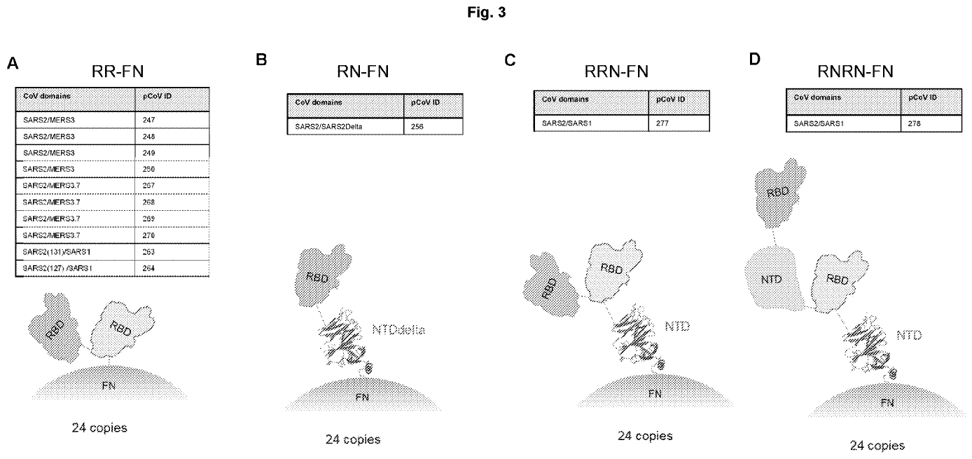

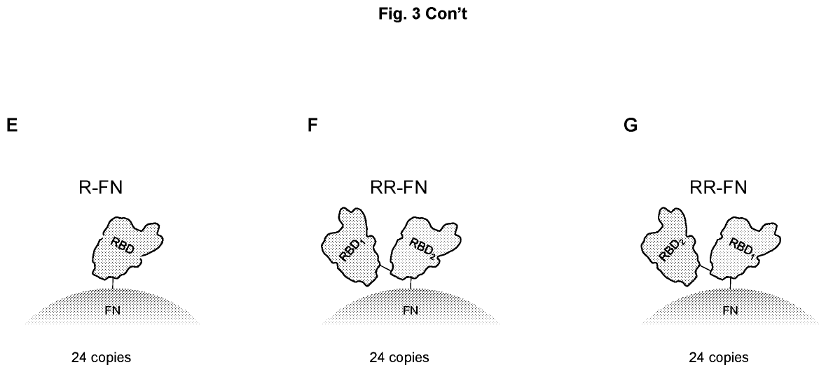

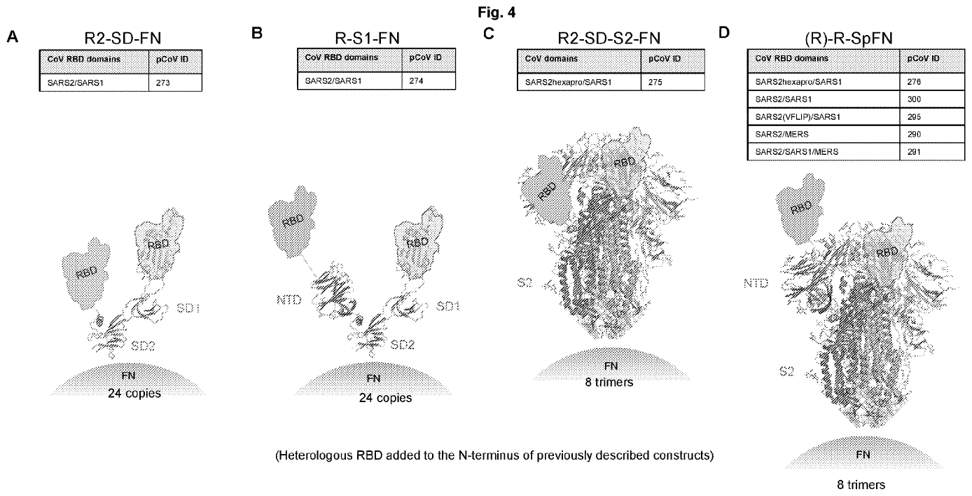

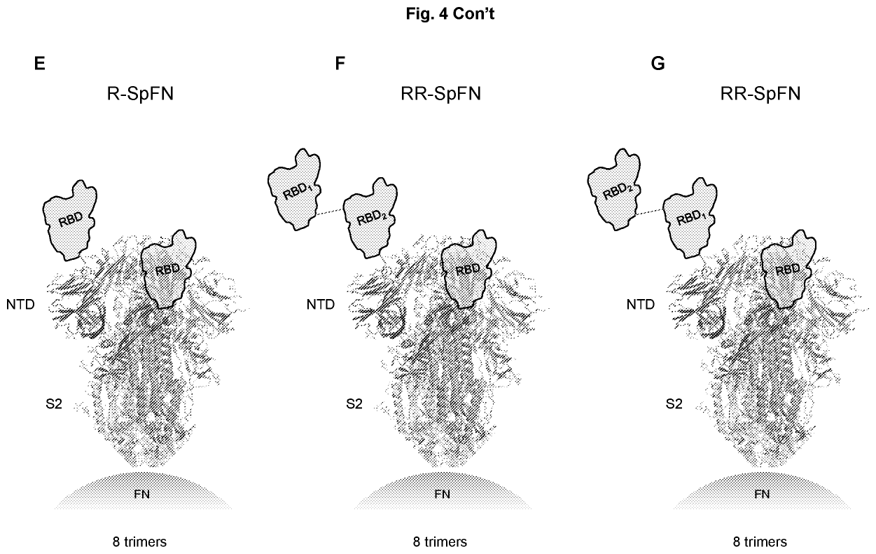

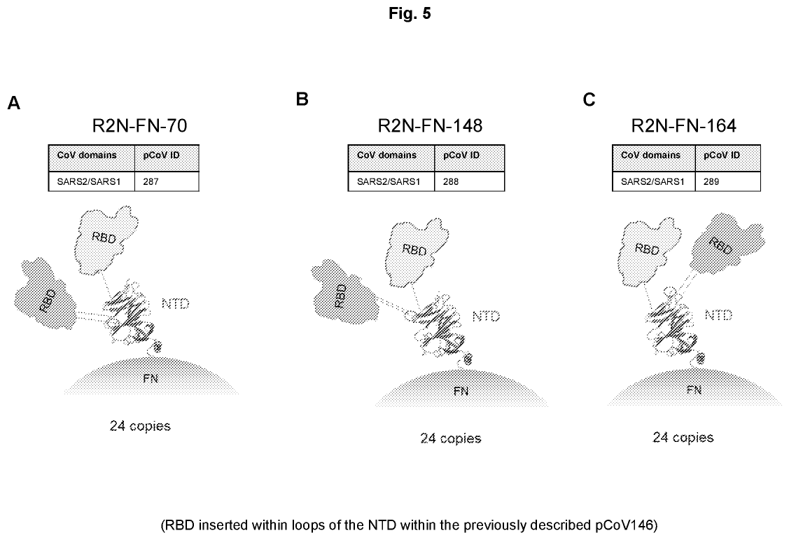

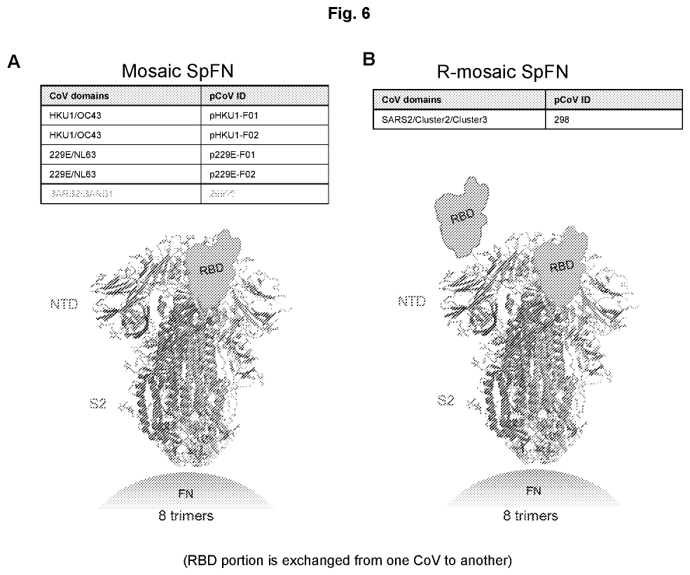

In general, the fusion proteins disclosed herein fall into one of four primary design formats: beads on a string (e.g., RN-FN, RRN-FN, RNRN-FN, R-FN, and RR-FN; see FIG. 3), domain fusions (R-S1-FN, R2-SD-S2-FN, and (R)-R-SpFN or RR-SpFN; see FIG. 4), loop insertions (R2N-FN; see FIG. 5), and domain swap (mosaic SpFN; see FIG. 6). Also included are SpFN formats. Exemplary fusion protein sequences are disclosed in Table 6 and Table 7. Exemplary mRNA sequences encoding the exemplary fusion protein sequences of Table 7 also are set forth in Table 7 (SEQ ID NOs: 552-566 and 575-582).

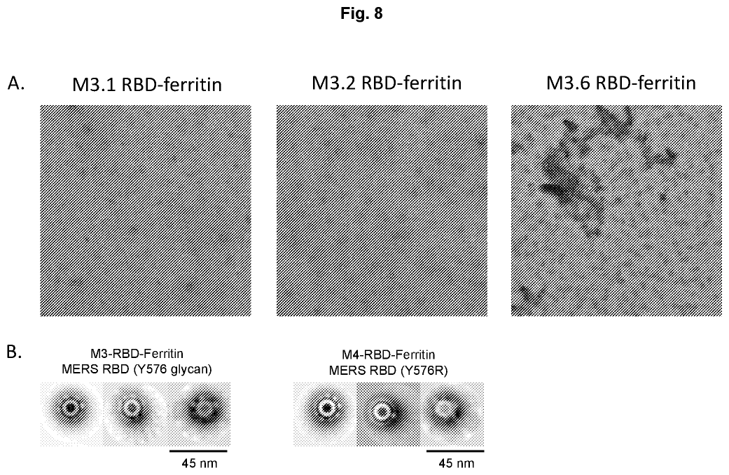

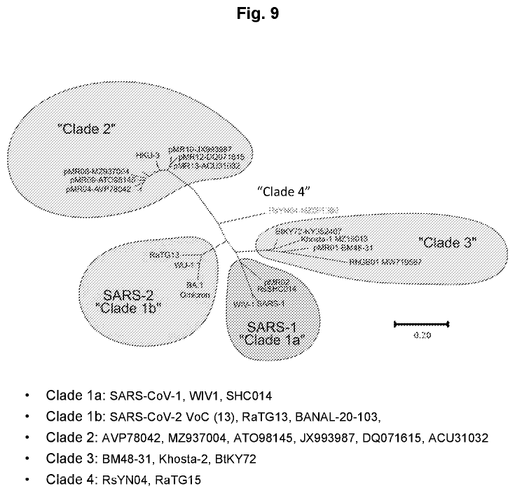

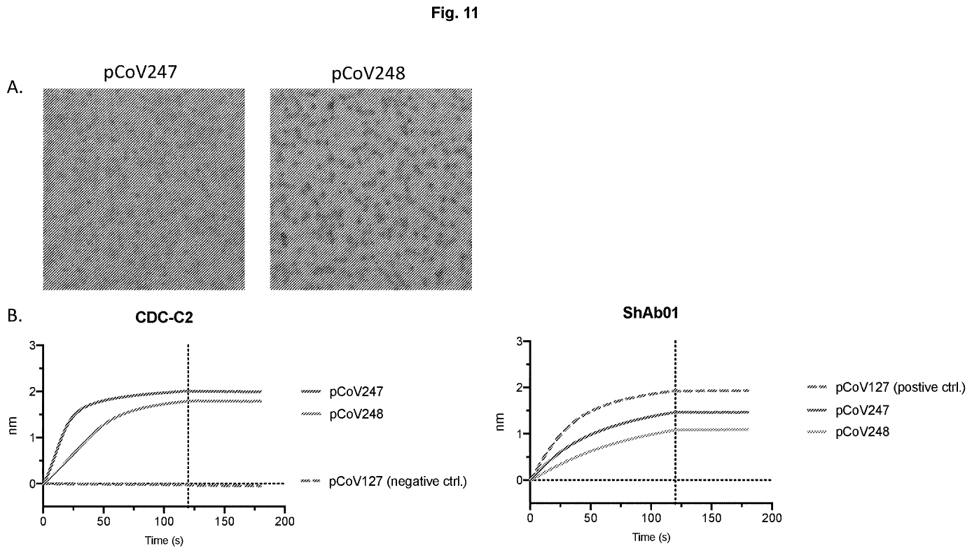

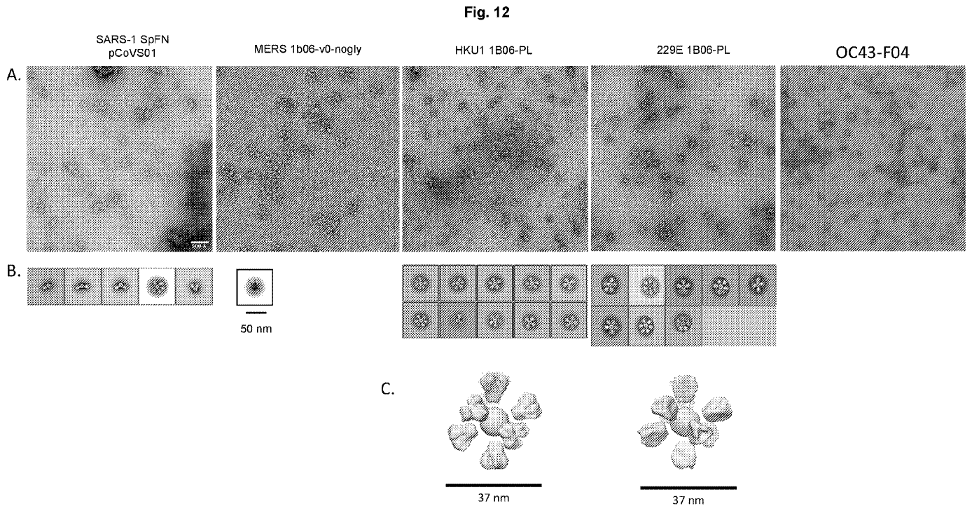

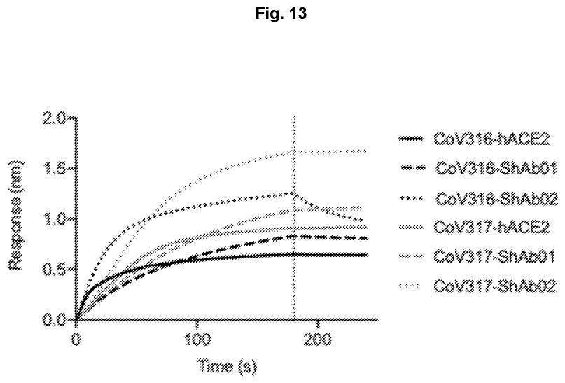

Negative-stain electron microscopy 3-dimensional reconstructions for select nanoparticles are shown in FIGS. 8, 11, and 12.

Exemplary nanoparticles and their respective designs are disclosed in Table 3, below.

TABLE 3
Exemplary Human CoV Domain Fusion Ferritin Nanoparticle Immunogens. All instances of “SARS-2”
refer to the WA-1 strain of SARS-CoV-2 unless otherwise specified’ SARS-1” refers to SARS-CoV-1.

| Class | Category | Features | ID | Construct |
|---|---|---|---|---|
| Beads on | RR-FN | SARS-2/MERS | pCoV247 | His8-3c-RBD(131)-GSGGSG- |
| a string | | | | M3RBD-Ferritin |
| | RR-FN | SARS-2/MERS | pCoV248 | His8-3c-RBD(127)-GSGGSG- |
| | | | | M3RBD-Ferritin |
| | RR-FN | MERS/SARS-2 | pCoV249 | His8-3c-M3RBD-GSGGSG- |
| | | | | RBD(131)-Ferritin |
| | RR-FN | MERS/SARS-2 | pCoV250 | His8-3c-M3RBD-GSGGSG- |
| | | | | RBD(127)-Ferritin |
| | RR-FN | SARS-2/MERS | pCoV267 | His8-His8-3c-RBD(131)- |
| | | | | GSGGSG-M3.7RBD-Ferritin |
| | RR-FN | SARS-2/MERS | pCoV268 | His8-3c-RBD(127)-GSGGSG- |
| | | | | M3.7RBD-Ferritin |
| | RR-FN | MERS/SARS-2 | pCoV269 | His8-3c-M3.7RBD-GSGGSG- |
| | | | | RBD(131)-Ferritin |
| | RR-FN | MERS/SARS-2 | pCoV270 | His8-3c-M3.7RBD-GSGGSG- |
| | | | | RBD(127)-Ferritin |
| | RR-FN | SARS-1/SARS-2 | pCoV263 | His8-3c-SARS1RBD-GSGGSG- |
| | | | | RBD(131)-Ferritin |
| | RR-FN | SARS-1/SARS-2 | pCoV264 | His8-3c-SARS1RBD-GSGGSG- |
| | | | | RBD(127)-Ferritin |
| | RN-FN | SARS-2 WA-1/SARS- | pCoV256 | His8-3c-RBD-GSGGSG- |
| | | 2delta | | NTD(delta)-Ferritin |
| | RRN-FN | SARS-1/SARS-2 | pCoV277 | His8-3c-SARS-1 RBD added to |
| | | | | N-term of SARS-1 RBD-NTD-Fn |
| | RNRN-FN | SARS-1/SARS-2 | pCoV278 | His8-3c-SARS-1 RBD-NTD |
| | | | | added to N-term of SARS-1 |
| | | | | RBD-NTD-Fn |
| | R-FN | SARS-2 Beta | | SARS-2 Beta RBD-Ferritin |
| | R-FN | SARS-2 Omicron | | SARS-2 Omicron BQ.1.1 RBD- |
| | | BQ.1.1 | | Ferritin |
| | R-FN | Khosta-2 | | Khosta-2 RBD-Ferritin |
| | R-FN | BANAL-20-247 | | BANAL-20-247 RBD-Ferritin |
| | RR-FN | SARS-2 Beta/SARS-2 | | SARS-2 Beta RBD-GSGGSG- |
| | | | | SARS-2 RBD-Ferritin |
| | RR-FN | SARS-2/SARS-2 Beta | | SARS-2 RBD-GSGGSG-SARS- |
| | | | | 2 Beta RBD-Ferritin |
| | RR-FN | SARS-2 Omicron | | SARS-2 Omicron BQ.1.1 RBD- |
| | | BQ.1.1/SARS-2 | | GSGGSG-SARS-2 RBD-Ferritin |
| | RR-FN | SARS-2/SARS-2 | | SARS-2 RBD-GSGGSG-SARS- |
| | | Omicron BQ.1.1 | | 2 Omicron BQ.1.1 RBD-Ferritin |
| | RR-FN | Khosta-2/SARS-2 | | Khosta-2 RBD-GSGGSG- |
| | | | | SARS-2 RBD-Ferritin |
| | RR-FN | SARS-2/Khosta-2 | | SARS-2 RBD-GSGGSG- |
| | | | | Khosta-2 RBD-Ferritin |
| | RR-FN | BANAL-20-247/SARS-2 | | BANAL-20-247 RBD- |
| | | | | GSGGSG-SARS-2 RBD-Ferritin |
| | RR-FN | SARS-2/BANAL-20-247 | | SARS-2 RBD-GSGGSG- |
| | | | | BANAL-20-247 RBD-Ferritin |
| | RR-FN | SARS-1/SARS-2 | | SARS-1 RBD-GSGGSG-SARS- |
| | | | | 2 RBD-Ferritin |
| | RR-FN | SARS-2/SARS-1 | | SARS-2 RBD-GSGGSG-SARS- |
| | | | | 1 RBD-Ferritin |
| Standard | SpFN | SARS-2 Omicron | | |
| SpFN | | BQ.1.1 | | |
| | SpFN | Khosta-2 | | |
| | SpFN | BANAL-20-247 | | |
| Domain | R2-SD-FN | SARS-1/SARS-2 | pCoV273 | His8-3c-SARS-1 RBD added to |
| fusions | | | | N-term of SARS-2 RBD-SD-Fn |
| | R-S1-FN | SARS-1/SARS-2 | pCoV274 | His8-3c-SARS-1 RBD added to |
| | | | | N-term of SARS-2 S1-Fn |
| | R2-SD-S2-FN | SARS-1/SARS-2 | pCoV275 | His8-3c-SARS-1 RBD added to |
| | | | | N-term of SARS-2 RBD-SD-S2-Fn |
| | R-SpFN | SARS-1/SARS-2 | pCoV276 | His8-3c-SARS-1 RBD added to |
| | | | | N-term of SARS-2 SpFN |
| | R-SpFN | MERS/SARS-2 | pCoV290 | His8-3c-MERS RBD added to |
| | | | | N-term of SARS-2 SpFN |
| | R-SpFN | SARS-1/SARS-2 | pCoV316 | SARS-CoV-2 SpFN-HexaPro |
| | | | | with SARS-CoV-1 RBD (131- |
| | | | | like) added |
| | R-SpFN | SARS-1/SARS-2 | pCoV317 | SARS-CoV-2 SpFN with SARS- |
| | | | | 1 RBD (131-like) added |
| | RR-SpFN | MERS/SARS-1/SARS-2 | pCoV291 | His8-3c-MERS RBD and |
| | | | | SARS-1 RBD added to N-term |
| | | | | of SARS-2 SpFN |
| | RR-SpFN | RsYN04/SARS-CoV-1/ | pCoV299 | RsYN04 RBD is added to the N- |
| | | SARS-CoV-2 | | terminus of SARS-CoV-1 RBD |
| | | | | connected to SARS-CoV-2 |
| | | | | SpFN i.e. RsYN04 added to the |
| | | | | N-term of pCoV300 |
| | R-SpFN | SARS-2 Omicron | | SARS-2 SpFN with SARS-2 |
| | | BQ.1.1/SARS-2 | | Omicron BQ.1.1 RBD |
| | R-SpFN | BANAL-20-247/SARS-2 | | SARS-2 SpFN with BANAL- |
| | | | | 20-247 RBD |
| | RR-SpFN | BANAL-20-247/ | | BANAL-20-247 RBD and |
| | | SARS-1/SARS-2 | | SARS-1 RBD added to N-term |
| | | | | of SARS-2 SpFN |
| | RR-SpFN | SARS-1/BANAL-20-247/ | | SARS-1 RBD and BANAL-20- |
| | | SARS-2 | | 247 RBD added to N-term of |
| | | | | SARS-2 SpFN |
| Loop | R2N-FN-70 | SARS-1/SARS-2 | pCoV287 | His8-3c-SARS-1 RBD inserted |
| insertions | | | | into NTD (loop at 70) in |
| | | | | pCoV146 |
| | R2N-FN-148 | SARS-1/SARS-2 | pCoV288 | His8-3c-SARS-1 RBD inserted |
| | | | | into NTD (loop at 148) in |
| | | | | pCoV146 |
| | R2N-FN-166 | SARS-1/SARS-2 | pCoV289 | His8-3c-SARS-1 RBD inserted |
| | | | | into NTD (loop at 166) in |
| | | | | pCoV146 |
| Domain | Mosaic | HKU1/OC43 | pHKU1-F01 | Replacement of RBD in HKU1 |
| swap | SpFN | | | SpFN with OC43 RBD |
| | Mosaic | HKU1/OC43 | pHKU1-F02 | Replacement of RBD-SD1 in |
| | SpFN | | | HKU1 SpFN with OC43 RBD-SD1 |
| | Mosaic | 229E/NL63 | pCoV229E-F01 | Replacement of RBD in 229E |
| | SpFN | | | SpFN with NL63 RBD |
| | Mosaic | 229E/NL63 | pCoV229E-F02 | Replacement of RBD-SD1 in |
| | SpFN | | | 229E SpFN with NL63 RBD-SD1 |
| | Mosaic | SARS-CoV-2/SARS-CoV-1 | pCoV298 | 1B06-PL SpFN (SARS-CoV-2 |
| | SpFN | | | WA-1 strain) with SARS-CoV-1 |
| | | | | RBD replacing the WA-1 RBD) |
| Domain | R-mosaic- | Cluster2 (OC43-like)/ | pCoV311 | Cluster2 RBD added to the N- |
| fusion | SpFN | MERS/ SARS-CoV-2 | | terminus of 1B06-PL SpFN |
| and | | | | (SARS-CoV-2 WA-1 strain) |
| domain | | | | with MERS-CoV RBD replacing |
| swap | | | | the WA-1 RBD) |
| | R-mosaic- | Cluster2 (OC43-like)/ | pCoV313 | Cluster2 RBD added to the N- |
| | SpFN | Cluster 3 (MERS-like)/ | | terminus of 1B06-PL SpFN |
| | | SARS-CoV-2 | | (SARS-CoV-2 WA-1 strain) |
| | | | | with cluster 3 (MERS-CoV-like) |
| | | | | RBD replacing the WA-1 RBD) |
| | R-mosaic- | SARS-CoV-2/SARS-CoV-1 | pCoV619 | Omicron BA.4/5 RBD added to |
| | SpFN | | | the N-terminus of 1B06-PL |
| | | | | SpFN (SARS-CoV-2 WA-1 |
| | | | | strain) with SARS-CoV-1 RBD |
| | | | | replacing the WA-1 RBD) |
| | R-mosaic- | SARS-CoV-2/SARS-CoV-1 | pCoV620 | Omicron BA.2.12.1 RBD added |
| | SpFN | | | to the N-terminus of 1B06-PL |
| | | | | SpFN (SARS-CoV-2 WA-1 |
| | | | | strain) with SARS-CoV-1 RBD |
| | | | | replacing the WA-1 RBD) |
| | RR-mosaic- | SARS-CoV-2/SARS-CoV-1 | pCoV658 | Omicron BA.2.12.1 RBD and |
| | SpFN | | | BA.4/5 RBD added to the N- |
| | | | | terminus of 1B06-PL SpFN |
| | | | | (SARS-CoV-2 WA-1 strain) |
| | | | | with SARS-CoV-1 RBD |
| | | | | replacing the WA-1 RBD) |
| | RR-mosaic- | SARS-CoV-2/SARS-CoV-1 | pCoV659 | NC_014470.BM4831.BGR.2008 |
| | SpFN | | | RBD and BA.4/5 RBD added to |
| | | | | the N-terminus of 1B06-PL |
| | | | | SpFN (SARS-CoV-2 WA-1 |
| | | | | strain) with SARS-CoV-1 RBD |
| | | | | replacing the WA-1 RBD) |
| | RR-mos | Khosta-2/BANAL-20-247/ | | Khosta-2 RBD and BANAL-20- |
| | SpFN | SARS-1/SARS-2 | | 247 RBD added to the N-term of |
| | | | | 1B06-PL SpFN (SARS-CoV-2 |
| | | | | WA-1 strain) with SARS-1 RBD |
| | | | | replacing the WA-1 RBD |
| | RR-mos | BANAL-20-247/ | | BANAL-20-247 RBD and |
| | SpFN | Khosta-2/SARS-1/SARS-2 | | Khosta-2 RBD added to the N- |
| | | | | term of 1B06-PL SpFN (SARS- |
| | | | | CoV-2 WA-1 strain) with |
| | | | | SARS-1 RBD replacing the |
| | | | | WA-1 RBD |
| | RR-mos | BANAL-20-247/ | | BANAL-20-247 RBD and |
| | SpFN | SARS-1/Khosta-2/SARS-2 | | SARS-1 RBD added to the N- |
| | | | | term of 1B06-PL SpFN (SARS- |
| | | | | CoV-2 WA-1 strain) with |
| | | | | Khosta-2 RBD replacing the |
| | | | | WA-1 RBD |
| | RR-mos | SARS1/BANAL-20-247/ | | SARS-1 RBD andBANAL-20- |
| | SpFN | Khosta-2/SARS-2 | | 247 RBD added to the N-term of |
| | | WA-1 | | 1B06-PL SpFN (SARS-CoV-2 |
| | | | | WA-1 strain) with Khosta-2 |
| | | | | RBD replacing the WA-1 RBD |
| | RR-mos | SARS-2 WA-1/ | | SARS-2 WA-1 RBD and |
| | SpFN | BANAL-20-247/ | | BANAL-20-247 RBD added to |
| | | SARS-1/SARS-2 | | the N-term of 1B06-PL SpFN |
| | | WA-1 | | (SARS-CoV-2 WA-1 strain) |
| | | | | with SARS-1 RBD replacing the |
| | | | | WA-1 RBD |
| | RR-mos | SARS-2 WA-1/SARS-1/ | | SARS-2 WA-1 RBD and SARS- |
| | SpFN | BANAL-20-247/ | | 1 RBD added to the N-term of |
| | | SARS-2 WA-1 | | 1B06-PL SpFN (SARS-CoV-2 |
| | | | | WA-1 strain) with BANAL-20- |
| | | | | 247 RBD replacing the WA-1 |
| | | | | RBD |

Nanoparticles as disclosed herein (administered directly or formed after administration of mRNA encoding component proteins) may bind to a human ACE-2 receptor. Additionally or alternatively, nanoparticles as disclosed herein may bind to a bat ACE2 protein, such as a protein from Chinese rufous horseshoe bat (R. sinicus 3364), Chinese rufous horseshoe bat (R. sinicus 1434), Intermediate horseshoe bat (R. affinis 787), Intermediate horseshoe bat (R. affinis 9479), Lander's horseshoe bat Subsaharan Africa (incl. Kenya) (R. landeri), Halcyon horseshoe bat West and Central African (R. alcyone), Greater horseshoe bat Asian, but also Europe and N. Africa (Rhinolophus ferrumequinum), and Least horseshoe bat (Rhinolophus pusillus). Additionally or alternatively, nanoparticles as disclosed herein may bind to a human DPP4 receptor.

The disclosed fusion proteins that self-assemble into the disclosed nanoparticles, including the nanoparticles described in Table 3 above and the fusion protein disclosed in Table 6 and Table 7 below, can be expressed alone or co-expressed (e.g., on two different plasmids) in suitable expression systems, which may include mammalian or eukaryotic expression systems. Some of the fusion proteins disclosed in Table 6 and Table 7 may comprise a histidine tag (i.e., His tag), which comprises a repeat of 5-10 histidine (H) residues or other tag sequences that may be useful in processing or purifying the protein, but which may ultimately be cleaved from the active protein before nanoparticle assembly. Alternatively, as noted above and discussed in more detail below, the disclosed fusion proteins that self-assemble into the disclosed nanoparticles, including the nanoparticles described in Table 3 above and the fusion protein disclosed in Table 6 and Table 7 below, can be encoded by mRNA molecules that when administered and expressed in vivo result in the formation of nanoparticles as disclosed herein.

All of the proteins disclosed in Table 6 and some of the proteins disclosed in Table 7 are exemplary nanoparticle-forming proteins that can form RBD-Ferritin or Spike-Ferritin nanoparticles. These sequences contain a set of alternate sequences to improve the stability and immunogenicity of the RBD-Ferritin or Spike-Ferritin constructs. This includes a stabilizing disulfide bond, a D614G mutation, a mutation to remove a glycan in the Spike at N165 to enable the RBD greater freedom of motion and allow the RBD to sit in the “up” and more exposed conformation, and a N234Q mutation to remove a glycan at 234 in the Spike to allow the RBD to sit in a more closed conformation. Additionally or alternatively, a glycan at N146 or N77 in the Ferritin sequence can improve and stabilize the Ferritin molecule.

The “beads on a string” fusion protein format can be used to create a nanoparticle with antigenic components from multiple coronaviruses such as SARS-CoV-2, SARS-CoV-1, Khosta-2, BANAL-20-247, HKU-1, MERS-CoV, 229E, NL63, OC43, or related coronaviruses including those identified from bats, camels, or pangolins. These embodiments can be utilized to create a pan-sarbecovirus vaccine, a pan-merbecovirus vaccine, a pan-sarbecovirus-merbecovirus vaccine, a pan-β-coronavirus vaccine, or pan-coronavirus vaccine. For example, multiple RBD, NTD, or a combination thereof “beads” comprised of different antigenic sequences can be provided together on a single “string” (i.e., in a single construct) to elicit broad immune responses against coronaviruses. For example, a “string” of antigens such as SARS-CoV-2-RBD-SARS-CoV-1-RBD-Khosta-2-RBD-BANAL-20-247-RBD or SARS-CoV-2-RBD-SARS-CoV-1-RBD-HKU-1-RBD-MERS-CoV-RBD-229E-RBD-NL63-RBD could be used with a “string” of antigens such as SARS-CoV-2-Omicron-BQ.1.1-RBD-SARS-CoV-1-RBD or SARS-CoV-2-RBD-pangolinSARS-CoV-1-RBD-OC43-RBD-camelMERS-CoV-RBD-229E-RBD-NL63-RBD to increase or focus the immune response to a specific pan-reactive or pan-protective immunity. The “beads on a string” may comprise, for example, 1-10 RBD, NTD, or sequences for both RBD and NTD domains in series, or, in other words, may comprise 1, 2, 3, 4, 5, 6, 7, 8, 9, or 10 RBDs, NTDs, or both. A linker sequence, including but not limited to the linker sequences disclosed in Table 1, may link one or more or each of the RBD and/or NTD sequences in series.

The “beads on a string” may also be added onto a SpFN or mos-SpFN molecule comprising an additional 1-10 RBDs, NTD, or both in series, or, in other words, may comprise 1, 2, 3, 4, 5, 6, 7, 8, 9, or 10 RBDs linked to the SpFN or mos-SpFN molecule. A linker sequence, including but not limited to a linker selected from the linker sequences disclosed in Table 1, may link one or more or each of the RBD and/or NTD sequences in series.

The “domain fusion” format of fusion protein can be used to create a nanoparticle with antigenic components from multiple coronaviruses such as SARS-CoV-2 and its variants and subvariants, including those of concern (e.g., Alpha, Beta, Delta, Omicron), SARS-CoV-1, Khosta-2, BANAL-20-247, HKU-1, MERS-CoV, 229E, NL63, OC43, or related coronaviruses including those identified from bats, camels, or pangolins. These embodiments can be utilized to create a pan-β-coronavirus vaccine, or pan-coronavirus vaccine. In this format, a heterologous RBD may be added to the N-terminus of a previously described construct. Exemplary constructs may comprise multiple (i.e., at least 2) RBD domains from different strains of coronavirus, along with other antigenic domains such as NTD, SD1, SD2, S-2P, or Hexapro domains.

The “loop insertion” format of fusion protein can be used to create a nanoparticle with antigenic components from multiple coronaviruses such as SARS-CoV-2, SARS-CoV-1, HKU-1, MERS-CoV, 229E, NL63, OC43, or related coronaviruses including those identified from bats, camels, or pangolins. These embodiments can be utilized to create a pan-β-coronavirus vaccine, or pan-coronavirus vaccine. In this format, one or more RBDs are attached or inserted into the loop domain of an NTD via a linker (e.g., a linker from Table 1). In some embodiments, at least two RBDs will be inserted, either in the same loop or different loops of the NTD. The RBDs may be from different strains or variants of coronavirus. The NTD may be from the same strain or variant as one or both of the RBDs, or the NTD may also be from a different strain or variant of coronavirus relative to one or both of the RBDs.

The “domain insertion” or “mosaic” format of fusion protein can be used to create a nanoparticle with antigenic components from multiple coronaviruses such as SARS-CoV-2 and its variants and subvariants, including those of concern (e.g., Alpha, Beta, Delta, Omicron), SARS-CoV-1, Khosta-2, BANAL-20-247, HKU-1, MERS-CoV, 229E, NL63, OC43, or other coronaviruses including those identified from bats, camels, or pangolins. Examples of useful coronaviruses include, but are not limited to sarbecoviruses (e.g., ZXC21, BANAL-20-247, Rf4092, Shaanxi2011, HeB2013, Rp3, Rs_672, HKU3-1, Rs4081, RmYN02, Rfl, Yun11, BM48-31, BB9904, Khosta-1, Khosta-2, RhGB01, BtKY72, RsYN04, RatG15 (Ra7909), SHC014, WIV1, LyRa3, Rs4084, Rs4231, BANAL-20-103, RaTG13, BANAL-20-52, Pangl7 (GX-P5L), or RshSTT182/200), merbecoviruses (e.g., MER1 (ErinaceusCoV/2012-174/GER/2012), MER2 (Neoromicia/5038), MER3 (HKU4 SM3A), MER4 (BatCoV-Ita2 206645-63), MER5 (BatCoV-Ital 206645-40), or HKU5), or a combination thereof. These embodiments can be utilized to create a pan-β-coronavirus vaccine, or pan-coronavirus vaccine. In this format, a spike protein or segment thereof is attached to a ferritin peptide, and one or more heterologous domains (e.g., RBD, NTD, or any combination thereof) are substituted in place of the native domain or added as an additional domain. For instance, a heterolgous RBD of one strain may be substituted for the native RBD of a given spike protein to form a “mosaic.” Additionally or alternatively, the RBD of a heterologous species or strain may be substituted in place of the native NTD of the spike protein to form a “mosaic.” Additionally or alternatively, one or more RBDs of a heterologous strain may be added to one end (i.e., C-terminus or N-terminus) of a native spike protein to form a mosaic. Multiple constructs can be combined together in a single nanoparticle by co-expression to produce a stable protein nanoparticles wherein the Spike trimer on the surface of the nanoparticle can be a heterologous mixture e.g. a protomer from WA-1 strain, a protomer from BA.4/5, and a protomer from Beta. These heterologous nanoparticles can also be encoded as mRNA constructs where mRNA molecules encoding different Spike-ferritin molecules can be encapsulated in a single lipid nanoparticle to facilitate heterologous nanoparticle formation within a vaccinated person. The heterologous nanoparticles could also be encoded within a single construct where exemplary cleavage sites are encoded between a given construct such as F2A (see, e.g., ncbi.nlm.nih.gov/pmc/articles/PMC4622431/).

In some embodiments, an immunogenic composition as described herein comprises antigenic coronavirus peptides from two or more coronavirus strains independently selected from Clade 1a, Clade 1b, Clade 2, Clade 3, and Middle East respiratory syndrome-related coronavirus (MERS-CoV) (or mRNA molecule(s) encoding them). In some embodiments, an immunogenic composition as described herein comprises antigenic coronavirus peptides from different coronavirus strains independently selected from WA-1, Beta, Omicron BQ.1.1, and XBB.1.5; a strain of SARS-CoV-1, BANAL20-247, Khosta2, and MERS-CoV (or mRNA molecule(s) encoding them). In some embodiments, an immunogenic composition as described herein comprises antigenic coronavirus peptides selected from the following combinations of strains (or mRNA molecule(s) encoding them): (i) two or more selected from WA-1, Beta, and Omicron BQ1.1 or XBB.1.5; (ii) one or more selected from WA-1, Beta, Omicron XBB.1.5, and Omicron BQ1.1, and strains of SARS-CoV-1; (iii) one or more selected from WA-1, Beta, Omicron XBB.1.5, and Omicron BQ1.1, and one or more selected from strains of SARS-CoV-1, BANAL20-247, and Khosta-2; (iv) one or more selected from WA-1, Beta, Omicron XBB.1.5, and Omicron BQ1.1, and one or more selected from strains of SARS-CoV-1, BANAL20-247, and Khosta-2, and MERS-CoV; and (v) two or more selected from strains of SARS-CoV-1, BANAL20-247, and Khosta-2.

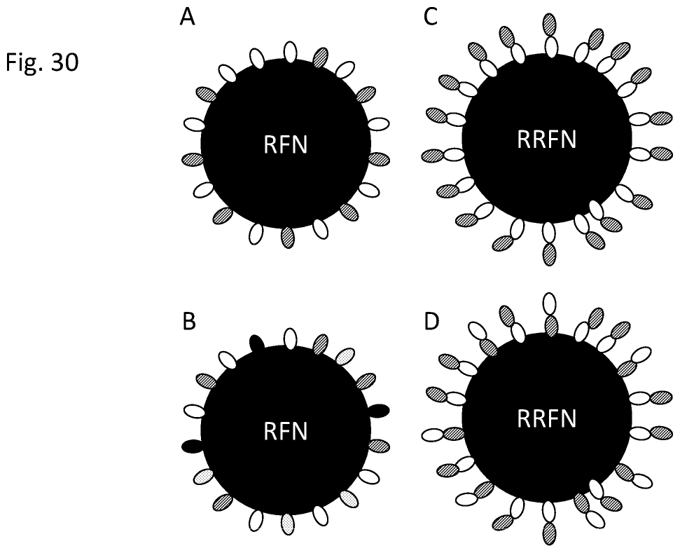

Fusion proteins can be designed with different antigen presentation formats. Fusion proteins may comprise a single antigenic coronavirus peptide (e.g., Spike protein and/or RBD antigens), alternatively conjugated to a ferritin moiety, or may include two or more antigenic coronavirus peptides in series conjugated to a nanoparticle-forming protein (e.g. a ferritin moiety) (also referred to herein as “multi-domained” fusion proteins). For fusion proteins comprising multiple antigenic coronavirus peptides, fusion proteins used in a single immunogenic composition (e.g., to form a single nanoparticle) may have different configurations of the antigenic coronavirus peptides (e.g., A-B and B-A) to provide antigens presented peripherally and laterally on the nanoparticle. In this context “peripherally” refers to epitopes on adjacent fusion proteins on the nanoparticle, while “laterally” refers to epitopes within the same fusion protein. For example, multi-domained fusion proteins (e.g., RBD-RBD or RBD-RBD-Spike, etc.) allow antibody binding peripherally and laterally. See, e.g., FIG. 30. Thus, to allow for both peripheral and lateral antibody binding, fusion proteins may be prepared to “pattern” the nanoparticle surface by changing the order of the component strains' antigens (e.g., RBDs), such that epitope combinations are adjacent both laterally and peripherally. For embodiments comprising spike proteins, “mosaic” formats can be designed, where the spike protein is from one strain and the RBD domain of the spike protein or an additional RBD domain is from a different coronavirus (mosSp, RmosSp). As an example, consider a fusion protein where the series of antigenic peptides are RBD-RBD-Spike (wherein Spike includes an RBD domain) taken from different strains of sarbecoviruses (A, B, and C, respectively, with Spike of strain C); additional fusion proteins could be RBD-RBD-mosSpike where the RBDs are (B, C, A, respectively, with Spike of strain C), and (C, A, B, respectively, with Spike of strain C), to allow the antigenic domains of each strain to be presented in each possible position of the fusion protein.

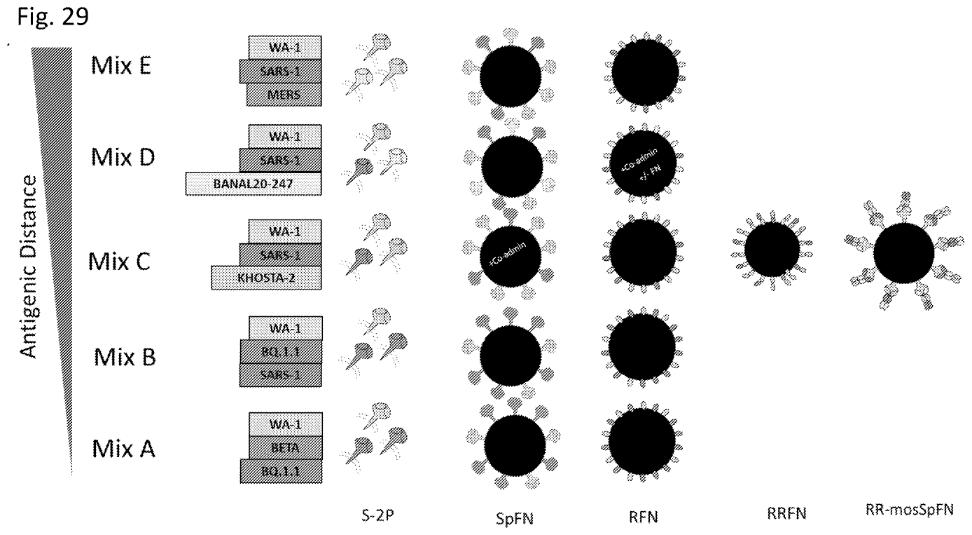

Implementing this approach, fusion proteins were designed according to the following antigen conjugation frameworks (“antigen presentations”) across various antigenic distance sets to provide multivalent nanoparticles displaying antigens of the selected antigenic distances to permit common and conserved epitopes to be optimally recognized and crosslinked by B cell receptors. Specifically, we created formulations containing up to five separate mRNA constructs (see FIG. 16, Panel A and Table 8 below) each encoding component strains from an antigenic distance set corresponding to the antigen presentation format (RFN, SpFN, RRFN, RR-SpFN) as shown in Table 8 and FIGS. 17 and 29.

Administration of an immunogenic composition as described herein comprising several different mRNA molecules (e.g., encoding different fusion proteins), either co-encapsulated in the same LNP or administered in the same composition after separate encapsulation in separate LNPs, result in co-expression of each encoded fusion protein (e.g., comprising ferritin-conjugated antigens). Ferritin-conjugated antigens produced in the same cell auto-assemble into a ferritin nanoparticle displaying the expressed fusion proteins on the same nanoparticle, resulting in what is referred to herein as a “mosaic nanoparticle” or “multivalent nanoparticle.”

For the examples of the present disclosure, seven strains with different levels of antigenic (i.e., sequence) distance that span the antigenic space of sarbecoviruses were selected. The strains include three antigenically distinct SARS-CoV-2 (Clade 1b) strains: Parental WA-1, Beta, Omicron XBB.1.5, and Omicron BQ.1.1; a SARS-CoV-1 (Clade 1a) strain (Frankfurt); two increasingly distant bat zoonotic coronaviruses (Clades 2 and 3): BANAL20-247 and Khosta2, respectively; and a Merbecovirus (MERS-CoV) strain, representing a non-ACE2 binding strain as an outlier (FIG. 16, Panel A). From this set of strains, the following sets of strains based on selected antigenic distance paradigms were designed: Low: Parental WA-1, Beta, Omicron BQ.1.1 (Mix A); Medium: Parental WA-1, Omicron BQ.1.1, SARS-CoV-1 (Mix B); Medium-High: Parental WA-1, SARS-CoV-1, Khosta-2 (Mix C); Medium-High: Parental WA-1, SARS-CoV-1, BANAL20-247 (Mix D); High: Parental WA-1, SARS-CoV-1, MERS-CoV (Mix E); High: SARS-CoV-1, Khosta-2, BANAL20-247 (Mix F).

As described in the examples, mRNA molecules were constructed encoding antigenic coronavirus peptides (e.g., spike and/or RBD antigens) from strains of these antigenic distance sets (with or without ferritin moieties), to obtain immunogenic compositions that provide multivalent presentation of antigens reflecting various antigenic distance paradigms. Thus, in some embodiments, an immunogenic composition as described herein comprises antigenic coronavirus peptides (or mRNA molecule(s) encoding them) selected from the following combinations of strains: (i) WA-1, Beta, and Omicron BQ.1.1 (or XBB.1.5), wherein the antigenic coronavirus peptides are RBD antigens comprised in RFN fusion proteins R(WA-1)FN, R(Beta)FN, and R(BQ1.1)FN, or are Spike antigens comprised in S-2P (e.g., S(WA-1)-2P, S(Beta)-2P, and S(BQ1.1)-2P) or SpFN fusion proteins Sp(WA-1)FN, Sp(Beta)FN, and Sp(BQ1.1)FN, or are mosaic antigens comprised in RmosSpFN or RRmosSpFN fusion proteins, or any combination thereof; (ii) WA-1, Omicron BQ.1.1 (or XBB1.5), and SARS-CoV-1, wherein the antigenic coronavirus peptides are RBD antigens comprised in RFN fusion proteins R(WA-1)FN, R(BQ 1.1)FN, and R(SARS-CoV-1)FN, or are Spike antigens comprised in S-2P (e.g., S(WA-1)-2P, S(BQ 1.1)-2P, and S(SARS-CoV-1)-2P) or SpFN fusion proteins Sp(WA-1)FN, Sp(BQ 1.1)FN, and Sp(SARS-CoV-1)FN, or are mosaic antigens comprised in RmosSpFN or RRmosSpFN fusion proteins, or any combination thereof; (iii) WA-1, SARS-CoV-1, and Khosta2, wherein the antigenic coronavirus peptides are RBD antigens comprised in RFN fusion proteins R(WA-1)FN, R(SARS-CoV-1)FN, and R(Khosta2)FN, or are Spike antigens comprised in S-2P (e.g., S(WA-1)-2P, S(SARS-CoV-1)-2P, and S(Khosta2)-2P) or SpFN fusion proteins Sp(WA-1)FN, Sp(SARS-CoV-1)FN, and Sp(Khosta2)FN, or are mosaic antigens comprised in RmosSpFN or RRmosSpFN fusion proteins, or any combination thereof; (iv) WA-1, SARS-CoV-1, and BANAL20-247, wherein the antigenic coronavirus peptides are RBD antigens comprised in RFN fusion proteins R(WA-1)FN, R(SARS-CoV-1)FN, and R(BANAL20-247)FN, or are Spike antigens comprised in S-2P (e.g., S(WA-1)-2P, S(SARS-CoV-1)-2P, and S(BANAL20-247)-2P) or SpFN fusion proteins Sp(WA-1)FN, Sp(SARS-CoV-1)FN, and Sp(BANAL20-247)FN, or are mosaic antigens comprised in RmosSpFN or RRmosSpFN fusion proteins, or any combination thereof; (v) WA-1, SARS-CoV-1, and MERS-CoV, wherein the antigenic coronavirus peptides are RBD antigens comprised in RFN fusion proteins R(WA-1)FN, R(SARS-CoV-1)FN, and R(MERS-CoV)FN, or are Spike antigens comprised in S-2P (e.g., S(WA-1)-2P, S(SARS-CoV-1)-2P, and S(MERS-CoV)-2P) or SpFN fusion proteins Sp(WA-1)FN, Sp(SARS-CoV-1)FN, and Sp(MERS-CoV)FN, or are mosaic antigens comprised in RmosSpFN or RRmosSpFN fusion proteins, or any combination thereof; and (vi) SAR-CoV-1, Khosta-2, and BANAL20-247, wherein the antigenic coronavirus peptides are RBD antigens comprised in RFN fusion proteins R(SARS-CoV-1)FN, R(Khosta2)FN, and R(BANAL20-247)FN, or are Spike antigens comprised in S-2P (e.g., S(SARS-CoV-1)-2P, S(Khosta2)-2P, and S(BANAL20-247)-2P) or SpFN fusion proteins Sp(SARS-CoV-1)FN, Sp(Khosta2)FN, and Sp(BANAL20-247)FN, or are mosaic antigens comprised in RmosSpFN or RRmosSpFN fusion proteins, or any combination thereof; (vii) Beta, Omicron BQ.1.1 (or XBB1.5), and SARS-CoV-1, wherein the antigenic coronavirus peptides are RBD antigens comprised in RFN fusion proteins R(Beta)FN, R(BQ 1.1)FN, and R(SARS-CoV-1)FN, or are Spike antigens comprised in S-2P (e.g., S(Beta)-2P, S(BQ 1.1)-2P, and S(SARS-CoV-1)-2P) or SpFN fusion proteins Sp(Beta)FN, Sp(BQ 1.1)FN, and Sp(SARS-CoV-1)FN, or are mosaic antigens comprised in RmosSpFN or RRmosSpFN fusion proteins, or any combination thereof; (viii) Beta, Omicron XBB.1.5, and SARS-CoV-1, wherein the antigenic coronavirus peptides are RBD antigens comprised in RFN fusion proteins R(Beta)FN, R(XBB 1.5)FN, and R(SARS-CoV-1)FN, or are Spike antigens comprised in S-2P (e.g., S(Beta)-2P, S(XBB 1.5)-2P, and S(SARS-CoV-1)-2P) or SpFN fusion proteins Sp(Beta)FN, Sp(XBB1.5)FN, and Sp(SARS-CoV-1)FN, or are mosaic antigens comprised in RmosSpFN or RRmosSpFN fusion proteins, or any combination thereof; (ix) Omicron BQ.1.1, SARS-CoV-1, and Khosta2, wherein the antigenic coronavirus peptides are RBD antigens comprised in RFN fusion proteins R(Omicron BQ.1.1)FN, R(SARS-CoV-1)FN, and R(Khosta2)FN, or are Spike antigens comprised in S-2P (e.g., S(Omicron BQ.1.1)-2P, S(SARS-CoV-1)-2P, and S(Khosta2)-2P) or SpFN fusion proteins Sp(Omicron BQ.1.1)FN, Sp(SARS-CoV-1)FN, and Sp(Khosta2)FN, or are mosaic antigens comprised in RmosSpFN or RRmosSpFN fusion proteins, or any combination thereof.

Any of the fusion proteins, nanoparticles, mRNA molecules and immunogenic compositions (e.g., vaccines) disclosed herein can be used for treating or preventing a coronavirus infection. Optimal doses and routes of administration may vary depending on the nature of the immunogenic composition (e.g., mRNA vs. nanoparticle), virus(es) being targeted, and subject being treated.

### F. Nucleic Acids Encoding Antigens and Nanoparticles

Although the foregoing discussion has focused on antigens, fusion proteins, and nanoparticles, it should be understood that the present disclosure encompasses mRNA molecules encoding antigenic coronavirus antigens as described herein, and immunogenic compositions comprising one or more mRNA molecules encoding antigenic coronavirus antigens as described herein, optionally wherein the mRNA molecule(s) are encapsulated or co-encapsulated in lipid nanoparticles (LNPs), as described in more detail below. Thus, additionally disclosed herein are nucleic acid-based vaccines (e.g., mRNA vaccines), priming agents (i.e., vaccine primers), and boosters that can be used to treat or prevent coronavirus infections such as COVID-19, which is caused by SARS-CoV-2, or to treat or prevent SARS-CoV-1 infection. For example, the disclosed nucleic acids can comprise DNA or mRNA that encodes any antigenic coronavirus peptide(s) or fusion protein as described herein (i.e., a fusion protein comprising a nanoparticle-forming peptide and an antigenic coronavirus peptide, which may optionally be connected by a linker). The antigenic coronavirus peptide encoded by the nucleic acid may comprise one or more fragments or full-length proteins derived from a coronavirus (e.g., SARS-CoV-2 or SARS-CoV-1), such as the S protein and, in particular, the RBD of the S protein, or any antigenic coronavirus peptide(s) as described herein.

Although the discussion herein focuses on mRNA, it should be understood that a nucleic acid of the disclosure may be RNA (including mRNA) or DNA. The nucleic acids of the disclosure may be single or double-stranded. In certain embodiments, the nucleic acid is RNA, e.g., mRNA.

In accordance with some aspects, the present disclosure provides an mRNA molecule comprising or consisting of a sequence selected from any one of SEQ ID NOs: 552-582 or a sequence at least 80% homologous thereto. In some embodiments, the mRNA molecule has a sequence comprising or consisting of a sequence with at least 80%, at least 85%, at least 90%, at least 95%, at least 96%, at least 97%, at least 98%, or at least 99% homology or identity to any one of SEQ ID NOs: 552-582. In some embodiments, the mRNA molecule has a sequence comprising any one of SEQ ID NOs: 552-582. In some embodiments, the mRNA molecule has a sequence consisting of any one of SEQ ID NOs: 552-582.

i. DNA Vaccines, Primers, and Boosters

DNA encoding a fusion protein as disclosed herein or an antigenic coronavirus peptide as disclosed herein (e.g., a coronavirus S protein or fragment or variant thereof) may be used as a vaccine, as a primer that can be administered prior to the administration of a nanoparticle or mRNA vaccine as disclosed herein, or as a booster after the administration of a nanoparticle or mRNA vaccine as disclosed herein. For example, the DNA can encode all, a fragment, or a variant of the RBD (or other antigenic peptide) of a coronavirus S protein (e.g., the S protein of SARS-CoV-2 or SARS-CoV-1). The DNA may be incorporated into a plasmid, which may comprise the necessary components (e.g., promoter) to express the DNA in vivo after administration to a subject, and the plasmid can be operably organized for expression in a mammal, such as a human.

### (1) Vectors

In one aspect, disclosed herein are vectors comprising a nucleic acid disclosed herein. In some embodiments, mRNAs as described herein may be cloned into a vector. Vectors include, but are not limited to, a plasmid, a phagemid, a phage derivative, an animal virus, and a cosmid. Vectors also include expression vectors, replication vectors, probe generation vectors, sequencing vectors, and vectors optimized for in vitro transcription (IVT).

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat.

Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or nucleic acids disclosed herein. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid or vector disclosed herein.

Vectors can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TransIT-RNA transfection Kit (Mirus, Madison, WI).

Chemical means for introducing a vector into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

ii. mRNA Vaccines

In some embodiments, the nucleic acids of the present disclosure are messenger RNAs (mRNAs). mRNAs can be modified or unmodified. mRNAs may contain one or more coding and non-coding regions. A coding region is alternatively referred to as an open reading frame (ORF). Non-coding regions in an mRNA include the 5′ cap, 5′ untranslated region (UTR), 3′ UTR, and a polyA tail. An mRNA can be purified from natural sources, produced using recombinant expression systems (e.g., in vitro transcription) and optionally purified, or chemically synthesised.

In certain embodiments, the mRNA comprises an ORF encoding an antigen of interest. In certain embodiments, the RNA (e.g., mRNA) further comprises at least one 5′ UTR, 3′ UTR, a poly(A) tail, and/or a 5′ cap.

A mRNA vaccine can be prepared by preparing an mRNA molecule that encodes any one or more of the antigenic coronavirus peptide(s) or fusion proteins disclosed herein. As a result of the self-assembling nature of the disclosed nanoparticles, expression of such an mRNA after administration to a subject will result in the formation of nanoparticles in vivo, and such nanoparticles can elicit an immunogenic response from the subject, such that the subject will produce coronavirus-specific antibodies. Accordingly the present disclosure provides mRNA, which can be used as vaccines, that encodes any or more of the antigenic coronavirus peptide(s) or fusion protein disclosed herein.

An mRNA as disclosed herein can encode any protein listed in Table 6 or Table 7. The mRNA may be linear mRNA or circular mRNA.

In some embodiments, an immunogenic composition as described herein comprises one or more mRNA molecules encoding at least two antigenic coronavirus peptides from different strains, optionally wherein the one or mRNA molecules are encapsulated or co-encapsulated in one or more lipid nanoparticles (LNPs). In some embodiments, a composition comprises one mRNA molecule encoding one fusion protein comprising at least two antigenic coronavirus peptides from different strains, optionally wherein the fusion protein further comprises a nanoparticle-forming peptide, wherein the mRNA molecule is encapsulated in a lipid nanoparticle (LNP). In some embodiments a composition comprises two or more mRNA molecules, each encoding at least one antigenic coronavirus peptide, optionally in a fusion protein with a nanoparticle-forming peptide, wherein each mRNA molecule is encapsulated in a separate lipid nanoparticle (LNP). In some embodiments a composition comprises two or more mRNA molecules, each encoding at least one antigenic coronavirus peptide, optionally in a fusion protein with a nanoparticle-forming peptide, wherein two or more mRNA molecules are co-encapsulated in the same lipid nanoparticle (LNP).

In any mRNA embodiments, the mRNA molecule may have one or more features selected from: a 5′ untranslated region (5′ UTR); a 3′ untranslated region (3′ UTR); a polyadenylation (poly(A)) sequence; a chemical modification, optionally wherein the chemical modification comprises N1-methylpseudouridine. Additionally or alternatively, the mRNA may be a self-replicating mRNA or a non-replicating mRNA. In one aspect the disclosure provides a composition comprising a nucleic acid as described herein comprising a nucleotide sequence (e.g., mRNA) encoding an antigen as described herein (e.g., a spike protein, RBD domain, and combinations thereof, etc.) or a fusion protein as described herein (e.g., RFN, RRFN, SpFN, mosSpFN, R-SpFN. R-mosSpFN, RR-SpFN, RR-mosSpFN, etc.).

Thus, the present disclosure provides a composition comprising one, two, three or four or more nucleic acid(s) (e.g., mRNA molecules) that comprises a nucleotide sequence encoding an antigenic coronavirus peptide as described herein (e.g., a sarbecovirus spike antigen or fragment thereof as described herein), optionally in a fusion protein comprising a nanoparticle-forming protein, such as a ferritin moiety. A single mRNA molecule may encode two or more antigenic coronavirus peptides as described herein, optionally in a fusion protein comprising a nanoparticle-forming protein, such as a ferritin moiety. Alternatively, a single mRNA molecule may encode only one antigenic coronavirus peptide as described herein, optionally in a fusion protein comprising a nanoparticle-forming protein, such as a ferritin moiety. Embodiments contemplated herein include embodiments where all component antigens are encoded by different mRNA molecules, embodiments where two or more or all of the component antigens are encoded by the same mRNA molecule, and all permutations and combinations thereof.

An immunogenic composition as disclosed herein may include a single type of mRNA molecule (e.g., mRNA molecules encoding the same antigenic coronavirus peptide(s)) or a combination of mRNA molecules (e.g., mRNA molecules encoding different coronavirus peptide(s)) formulated in the same composition. Additionally or alternatively, a composition as described herein may comprise mRNA molecules encoding only one antigenic coronavirus peptide, optionally for use in combination with another such monovalent composition, or for use in combination with a multivalent composition as described herein (e.g., a combination of compositions for simultaneous, separate, or sequential administration). Embodiments contemplated herein include embodiments where two or more or all mRNA molecules are formulated in the same composition (optionally encapsulated or co-encapsulated in the same or separate LNPs) and embodiments where two more or all of the mRNA molecules are formulated in separate compositions. In some embodiments, all mRNA molecules are formulated in the same composition (optionally encapsulated in separate LNPs or co-encapsulated in the same LNPs). In other embodiments, each mRNA molecule is formulated in a separate composition. In some embodiments, two or more mRNA molecules are formulated in one composition (optionally encapsulated in separate LNPs or co-encapsulated in the same LNPs) and one or more additional mRNA molecules are formulated in a second composition (optionally encapsulated in separate LNPs or co-encapsulated in the same LNPs). In some embodiments, one, two, three, four or more mRNA molecules as described herein are formulated in one composition, or in two, three, four or more compositions each having any combination or subcombination thereof.

An mRNA composition as described herein may optionally include one or more additional components, such as one or more small molecule immunopotentiators (e.g., TLR agonists).

An mRNA composition as described herein may optionally include a delivery system for a nucleic acid (e.g., mRNA), such as a liposome, an oil-in-water emulsion, or a microparticle. In some embodiments, in a composition as described herein comprising mRNA, the mRNA is encapsulated in a lipid nanoparticle (LNP), such as in an LNP formulation.

5′Cap (1)

An mRNA 5′ cap can provide resistance to nucleases found in most eukaryotic cells and promote translation efficiency. Several types of 5′ caps are known. A 7-methylguanosine cap (also referred to as “m⁷G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference. [0155]5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-o methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

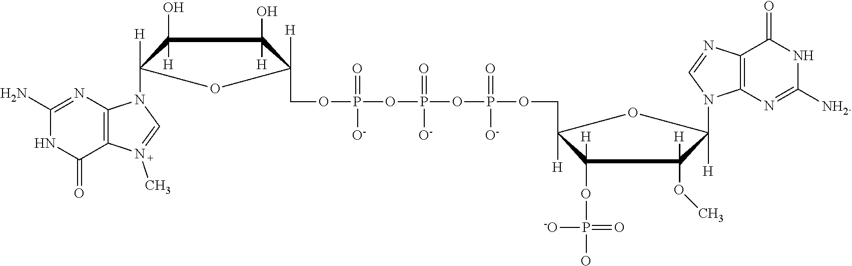

### (2) Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 25) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 26 and reproduced below:

(SEQ ID NO: 26)
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACG.

In some embodiments, the 3′UTR comprises a nucleic acid sequence set forth in SEQ ID NO: 27 and reproduced below:

(SEQ ID NO: 27)
CGGGUGGCAUCCCUGUGACCCCUCCCCAGUGCCUCUCCUGGCCCUGGAAG
UUGCCACUCCAGUGCCCACCAGCCUUGUCCUAAUAAAAUUAAGUUGCAU
C.

The 5′ UTR and 3′UTR are described in further detail in WO2012/075040, incorporated herein by reference.

### (3) Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, which are different from an adenosine nucleotide). In certain embodiments, the poly(A) tail comprises the sequence

(SEQ ID NO: 28)
AAAAAAAAAAAAAAAAAAAAAAAAAAAAAAGCAUAUGACUAAAAAAAAAA
AAAAAAAAAAAAAAAAAAAAAAAAAAAAAAAAAAAAAAAAAAAAAAAAAA
AAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A)polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### (4) Chemical Modification

The mRNA disclosed herein may be modified or unmodified. Typically, the mRNA comprises at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

(5) mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 rNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

iii. Other RNA Vaccines

### (1) Self-Replicating RNA, Trans-Replicating RNA and Non-Replicating RNA

Typically, the nucleic acid molecules described herein are non-replicating RNAs. However, the nucleic acid molecules described herein may alternatively be self-replicating RNAs or trans-replicating RNAs.

### (2) Self-Replicating RNA

Self-replicating (or self-amplifying) RNA can be produced by using replication elements derived from, e.g., alphaviruses, and substituting the structural viral proteins with a nucleotide sequence encoding a protein of interest (e.g., a sarbecovirus spike antigen). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) a Spike polypeptide antigen, as disclosed herein. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

### (3) Trans-Replicating RNA

Trans-replicating (or trans-amplifying) RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., a Spike protein described herein). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### (4) Non-Replicating RNA

Non-replicating (or non-amplifying) RNA is an RNA without the ability to replicate itself.

### G. LNPs

In certain embodiments, an immunogen composition as described herein comprises a lipid nanoparticle (LNP) encapsulated one or more mRNA molecules as described herein. The mRNA can be encapsulated in a lipid nanoparticle (LNP) through methodology known in the art, such as a modified ethanol-drop nanoprecipitation process. In brief, ionizable, structural, helper and polyethylene glycol lipids can be mixed with mRNA in acetate buffer, pH 5.0, at a given ratio of lipids:mRNA. The mixture can be neutralized with Tris-Cl pH 7.5, sucrose added as a cryoprotectant, sterile filtered and stored frozen at −70° C. until further use. The mRNA and LNP can be as follows: The lipid nanoparticle contains RNA, an ionizable lipid, ((4-hydroxybutyl)azanediyl)bis(hexane-6,1-diyl)bis(2-hexyldecanoate)), a PEGylated lipid, 2-[(polyethylene glycol)-2000]-N,N-ditetradecylacetamide and two structural lipids (1,2-distearoyl-sn-glycero-3-phosphocholine (DSPC]) and cholesterol). Those skilled in the art will understand that this is merely one exemplary way of formulating mRNA and that other methods and formulating agents (e.g., other lipids) used in the art may be suitable as well. Parallel methodology can be used to practice other embodiments of mRNA vaccines contemplated herein.

In certain embodiments, the composition of the disclosure (e.g., the composition comprising a nucleic acid of the disclosure) further comprises a lipid nanoparticle (LNP). In certain embodiments, the nucleic acid of the disclosure is encapsulated in the LNP.

The LNPs of the disclosure may comprise four categories of lipids: (i) an ionizable lipid (e.g., a cationic lipid); (ii) a PEGylated lipid; (iii) a cholesterol-based lipid, and (iv) a helper lipid.

i. Ionizable Lipids

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the cationic lipid is OF-02:

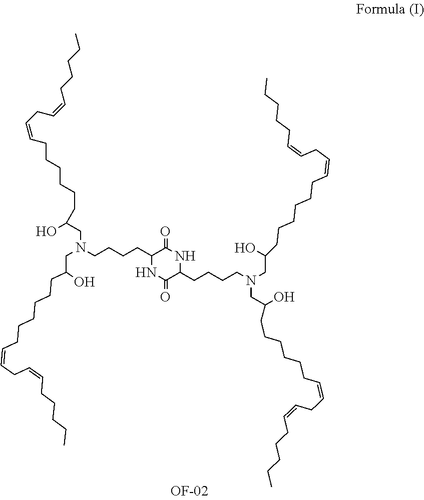 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC]

OF-02 is a non-degradable structural analog of OF-Deg-Lin. OF-Deg-Lin contains degradable ester linkages to attach the diketopiperazine core and the doubly-unsaturated tails, whereas OF-02 contains non-degradable 1,2-amino-alcohol linkages to attach the same diketopiperazine core and the doubly-unsaturated tails (Fenton et al., Adv Mater. (2016) 28:2939; U.S. Pat. No. 10,201,618). An exemplary LNP formulation herein, Lipid A, contains OF-2.

In some embodiments, the cationic lipid is cKK-E10 (Dong et al., PNAS (2014) 111(11):3955-60; U.S. Pat. No. 9,512,073):

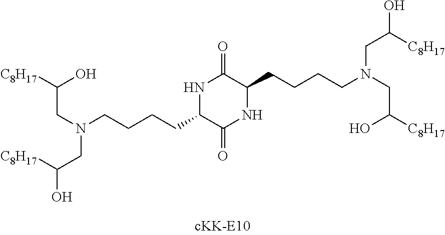

An exemplary LNP formulation herein, Lipid B, contains cKK-E10.

In some embodiments, the cationic lipid is GL-HEPES-E3-E10-DS-3-E18-1 (2-(4-(2-((3-(Bis((Z)-2-hydroxyoctadec-9-en-1-yl)amino)propyl)disulfaneyl)ethyl)piperazin-1-yl)ethyl 4-(bis(2-hydroxydecyl)amino)butanoate) (WO2022/221688), which is a HEPES-based disulfide cationic lipid with a piperazine core, having the Formula III:

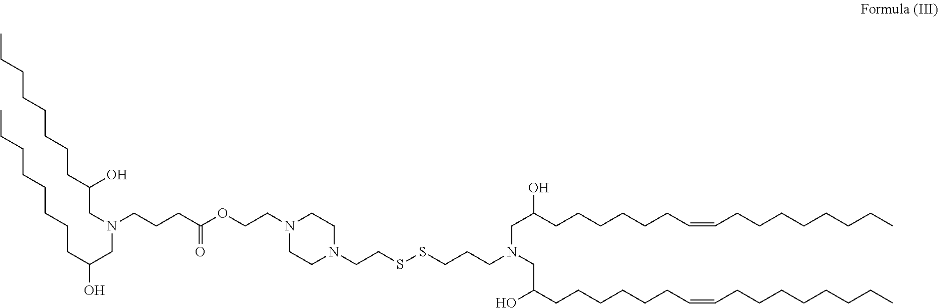 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

An exemplary LNP formulation herein, Lipid C, contains GL-HEPES-E3-E10-DS-3-E18-1. Lipid C has the same composition as Lipid A or Lipid B but for the difference in the cationic lipid.

In some embodiments, the cationic lipid is GL-HEPES-E3-E12-DS-4-E10 (2-(4-(2-((3-(bis(2-hydroxydecyl)amino)butyl)disulfaneyl)ethyl)piperazin-1-yl)ethyl 4-(bis(2-hydroxydodecyl)amino)butanoate) (WO2022/221688), which is a HEPES-based disulfide cationic lipid with a piperazine core, having the Formula IV:

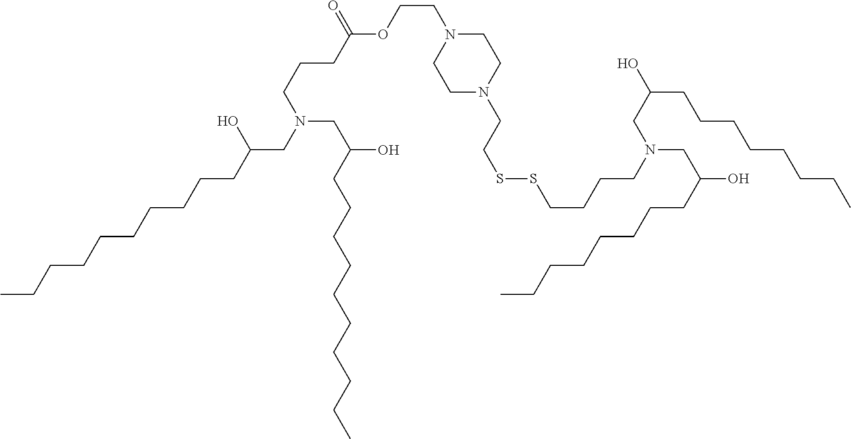 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

An exemplary LNP formulation herein, Lipid D, contains GL-HEPES-E3-E12-DS-4-E10. Lipid D has the same composition as Lipid A or Lipid B but for the difference in the cationic lipid.

In some embodiments, the cationic lipid is GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate) (WO2022/221688), which is a HEPES-based disulfide cationic lipid with a piperazine core, having the Formula V:

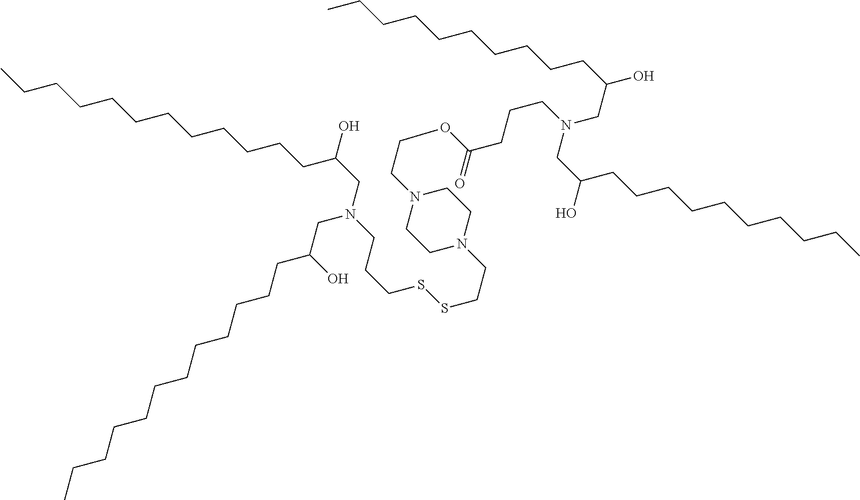 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC]

An exemplary LNP formulation herein, Lipid E, contains GL-HEPES-E3-E12-DS-3-E14. Lipid E has the same composition as Lipid A or Lipid B but for the difference in the cationic lipid.

The cationic lipids GL-HEPES-E3-E10-DS-3-E18-1 (111), GL-HEPES-E3-E12-DS-4-E10 (IV), and GL-HEPES-E3-E12-DS-3-E14 (V) can be synthesized according to the general procedure set out in Scheme 1:

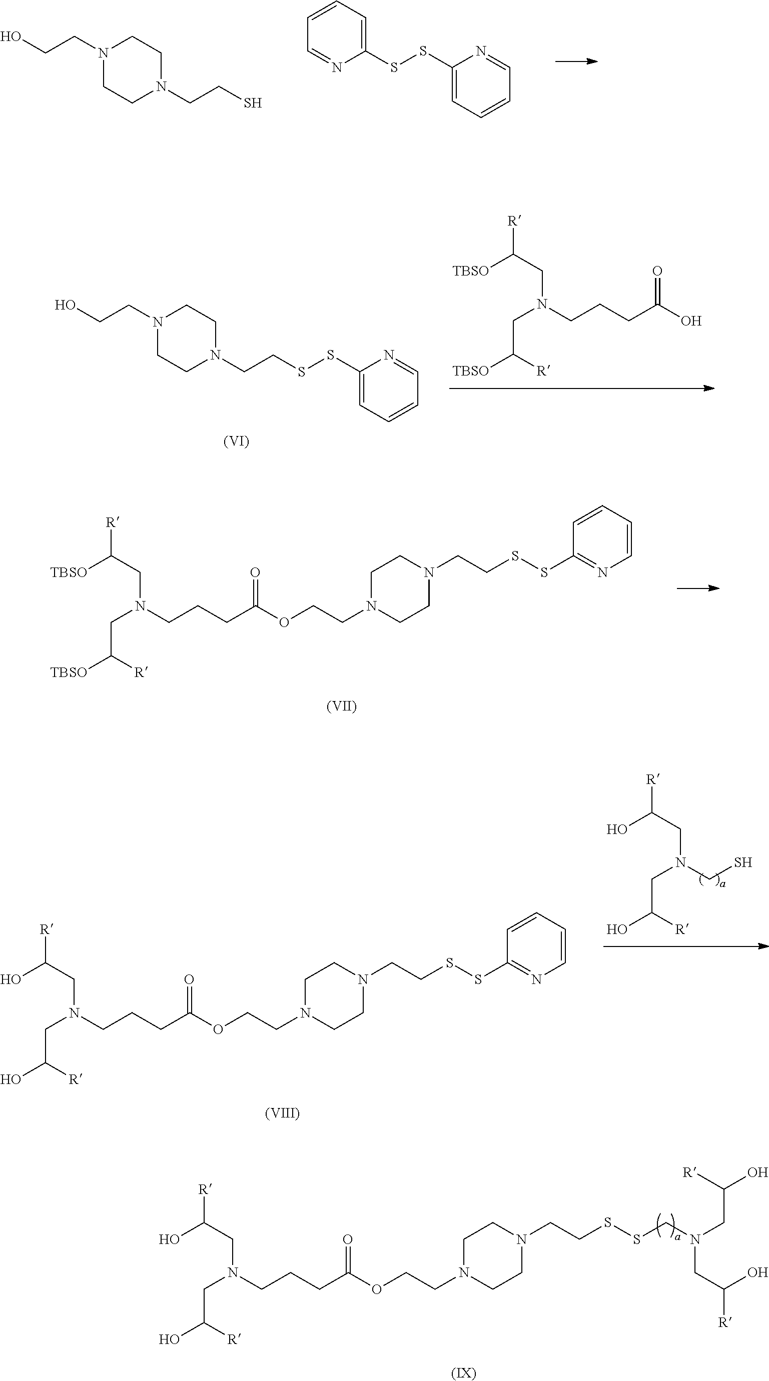

In some embodiments, the cationic lipid is MC3, having the Formula VI:

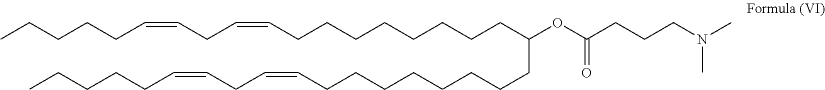 [SMILES: CCCCC/C=C\C/C=C\CCCCCCCCC(CCCCCCCC/C=C\C/C=C\CCCCC)OC(=O)CCCN(C)C]

In some embodiments, the cationic lipid is SM-102 (9-heptadecanyl 8-{(2-hydroxyethyl)[6-oxo-6-(undecyloxy)hexyl]amino}octanoate), having the Formula VII:

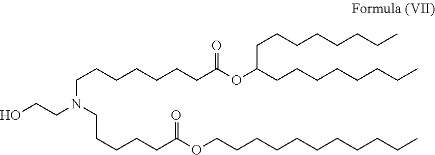 [SMILES: CCCCCCCCCCCOC(=O)CCCCCN(CCO)CCCCCCCC(=O)OC(CCCCCCCC)CCCCCCCC]

In some embodiments, the cationic lipid is ALC-0315 [(4-hydroxybutyl)azanediyl]di(hexane-6,1-diyl) bis(2-hexyldecanoate), having the Formula VIII:

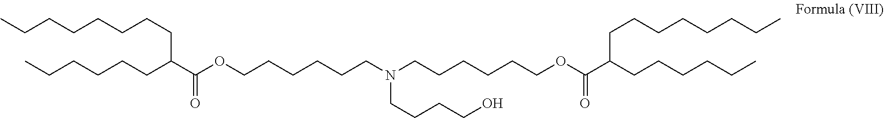 [SMILES: CCCCCCCCC(CCCCCC)C(=O)OCCCCCCN(CCCCO)CCCCCCOC(=O)C(CCCCCC)CCCCCCCC]

In some embodiments, the cationic lipid is cOrn-EE1, having the Formula IX:

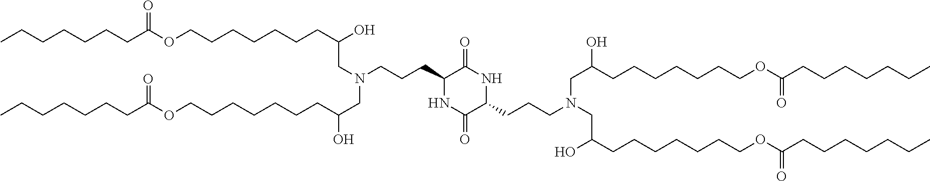 [SMILES: CCCCCCCC(=O)OCCCCCCCC(O)CN(CCC[C@@H]1NC(=O)[C@@H](CCCN(CC(O)CCCCCCCOC(=O)CCCCCCC)CC(O)CCCCCCCOC(=O)CCCCCCC)NC1=O)CC(O)CCCCCCCOC(=O)CCCCCCC]

In some embodiments, the cationic lipid may be selected from the group comprising cKK-E10; OF-02; [(6Z,9Z,28Z,31Z)-heptatriaconta-6,9,28,31-tetraen-19-yl]4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl](Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl]N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (3060i10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-016B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); GL-HEPES-E3-E10-DS-3-E18-1; GL-HEPES-E3-E12-DS-4-E10; GL-HEPES-E3-E12-DS-3-E14; and combinations thereof.

In some embodiments, the cationic lipid is IM-001, having the Formula X (EP23306049.0):

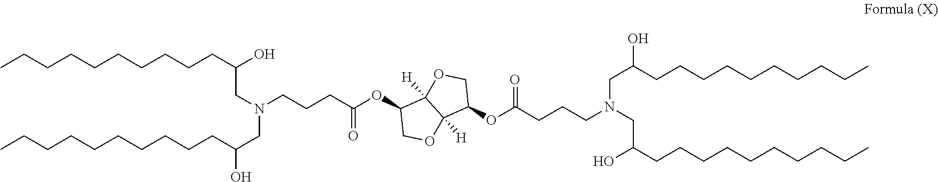 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

In some embodiments, the cationic lipid is IS-001, having the Formula XI (EP23306049.0):

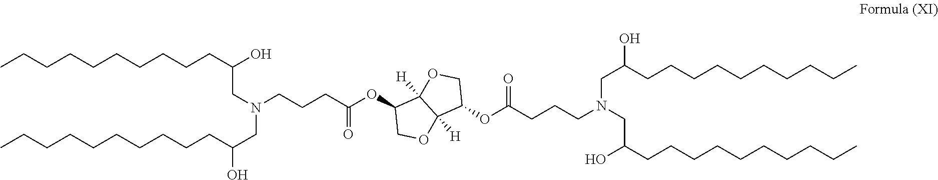 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

In some embodiments, the cationic lipid is biodegradable.

In some embodiments, the cationic lipid is not biodegradable.

In some embodiments, the cationic lipid is cleavable.

In some embodiments, the cationic lipid is not cleavable.

Cationic lipids are described in further detail in Dong et al. (PNAS. 111(11):3955-60. 2014); Fenton et al. (Adv Mater. 28:2939. 2016); U.S. Pat. Nos. 9,512,073; and 10,201,618, each of which is incorporated herein by reference.

ii. PEGylated Lipids

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268(1):235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)](C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

iii. Cholesterol-Based Lipids

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3β-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3β-ol); lathosterol (5α-cholest-7-en-3β-ol); diosgenin ((3β,25R)-spirost-5-en-3-ol); campesterol (campest-5-en-3β-ol); campestanol (5a-campestan-3b-ol); 24-methylene cholesterol (5,24(28)-cholestadien-24-methylen-3β-ol); cholesteryl margarate (cholest-5-en-3β-yl heptadecanoate); cholesteryl oleate; cholesteryl stearate and other modified forms of cholesterol. In some embodiments, the cholesterol-based lipid used in the LNPs is cholesterol.

iv. Helper Lipids

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

In other embodiments, the present LNPs comprise (i) SM-102; (ii) DMG-PEG2000; (iii) cholesterol; and (iv) DSPC.

In yet other embodiments, the present LNPs comprise (i) ALC-0315; (ii) ALC-0159; (iii) cholesterol; and (iv) DSPC.

v. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A: B: C: D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises: a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%); a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%); a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 1%, 6%, 17%, 18%, 19%, 20%, 21%, 22%, 23%, 24%, 25%, 26%, 27%, 28%, 29%, 30%, 31%, 32%, 33%, 34%, or 35%), wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the LNP of the disclosure comprises: a cationic lipid at a molar ratio of 45 to 50%; a PEGylated lipid at a molar ratio of 1.5 to 1.7%; a cholesterol-based lipid at a molar ratio of 38 to 43%; and a helper lipid at a molar ratio of 9 to 10%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises DLin-MC3-DMA (MC3) at a molar ratio of 50%; DMG-PEG2000 at a molar ratio of 1.5%; cholesterol at a molar ratio of 38.5%; and DSPC at a molar ratio of 10%. This LNP formulation is designated “Lipid F” herein.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: IS-001 at a molar ratio of 40%; DMG-PEG2000 at a molar ratio of 1.5%; cholesterol at a molar ratio of 28.5%; and DOPE at a molar ratio of 30%.

In some embodiments, the LNP formulation is as defined for “Lipid A”, “Lipid B” or “Lipid D”.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

vi. Nucleic Acids within LNPs

The LNP compositions described herein may comprise a nucleic acid (e.g., a mRNA) of the present disclosure.

Where desired, the LNP may be multivalent. In some embodiments, the LNP may carry nucleic acids, such as mRNAs, which encode more than one polypeptide of the present disclosure, such as two, three, four, five, six, seven, or eight polypeptides. For example, the LNP may carry multiple nucleic acids of the present disclosure (e.g., mRNA), each encoding a different polypeptide of the disclosure; or carry a polycistronic mRNA that can be translated into more than one polypeptide of the disclosure (e.g., each antigen-coding sequence is separated by a nucleotide linker encoding a self-cleaving peptide such as a 2A peptide). An LNP carrying different nucleic acids (e.g., mRNA) typically comprises (encapsulate) multiple copies of each nucleic acid. For example, an LNP carrying or encapsulating two different nucleic acids typically carries multiple copies of each of the two different nucleic acids.

In some embodiments, a single LNP formulation may comprise multiple kinds (e.g., two, three, four, five, six, seven, eight, nine, ten, or more) of LNPs, each kind carrying a different nucleic acid (e.g., mRNA).

When the nucleic acid is mRNA, the mRNA may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridine), 2′-fluoro ribonucleotides, and 2′-methoxy ribonucleotides, and/or phosphorothioate bonds). The mRNA molecule may comprise a 5′ cap and a polyA tail.

vii. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

viii. Processes for Making Present LNP Compositions

The present LNPs can be prepared by various techniques presently known in the art. For example, multilamellar vesicles (ILV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MILVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and PCT/US2020/043223 (filed Jul. 23, 2020) and can be used to practice the present disclosure. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm) or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of between 50% and 99%; or greater than about 60, 65, 70, 75, 80, 85, 90, 92, 95, 98, or 99%. Typically, lipid nanoparticles for use herein have an encapsulation efficiency of at least 90% (e.g., at least 91, 92, 93, 94, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present disclosure contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. An exemplary process for making and purifying mRNA is described in Example 1. In this process, in an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 3.5 or 4.5.

An exemplary, nonlimiting process for making an mRNA-LNP composition is described in Example 1. The process involves mixing of a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### H. Nucleic Acid Formulations and Adjuvants

The nucleic acid vaccines, primers, and boosters disclosed herein may be formulated for systemic administration via parenteral delivery. Parenteral administration includes intravenous, intra-arterial, subcutaneous, intradermal, intraperitoneal, or intramuscular injection or infusion. Formulations for parenteral administration may include sterile aqueous solutions, which may also contain buffers, diluents and other pharmaceutically acceptable additives known to the skilled artisan. For intravenous use, the total concentration of solutes may be controlled to render the preparation isotonic. Intravenous, intra-arterial, subcutaneous, or intramuscular injection are preferred routes of administration. Additionally or alternatively, the disclosed vaccines can be formulated for intranasal administration or contact with other mucosa membranes.

Formulations of the nucleic acids for injection may be presented in unit dosage form, e.g., in ampules, or in multi-dose containers, optionally with an added preservative. The formulations may take such forms as suspensions, solutions or emulsions in oily or aqueous vehicles, and may contain formulatory agents such as suspending, stabilizing and/or dispersing agents. The formulations may comprise any suitable pharmaceutically acceptable excipients.

Commonly, nucleic acids that are administered to a subject are formulated in a lipid composition, such as a lipid nanoparticle, as discussed above.

### I. Vaccine Adjuvants

The disclosed immunogenic compositions (e.g., comprising fusion proteins, nanoparticles or mRNA molecule(s) as described herein) can comprise an adjuvant to improve immune responses and promote protective responses. An adjuvant is an ingredient used in some vaccines that helps create a stronger immune response in people receiving the vaccine. Adjuvants help the body to produce an immune response strong enough to protect the person from the disease he or she is being vaccinated against.

The present disclosure provides vaccine formulations that contain any of (or a combination of) the disclosed antigens and/or nanoparticles and at least one adjuvant selected from the group consisting of ALFQ, alhydrogel, and combination thereof.

The adjuvant ALFQ was developed by the U.S. Army, and is an Army-Liposome-Formulation (ALF) containing high amounts of cholesterol together with the QS21 saponin (ALFQ). ALFQ has been used in numerous animal studies and with a variety of immunogens, and has shown effectiveness in eliciting robust immune responses. In contrast to some adjuvants, ALFQ tends to elicit a balanced Th1/Th2 immune response avoiding a skewed immune response that has been implicated in vaccine associated enhanced respiratory disease (VAERD). In some embodiments, the ALFQ adjuvant is a liposomal formulation containing monophosphoryl lipid A (MPLA) and QS-21 saponin. In some embodiments, the ALFQ liposomes may contain about 600 μg/mL monophosphoryl 3-deacyl lipid A (3D-PHAD) and about 300 μg/mL QS-21. To make the ALFQ, in one exemplary embodiment, 14.7 mL of ALF55 (containing 1.236 mg/mL 3D-PHAD) may be diluted with 6.5 mL of isotonic Sorensen's PBS pH 6.15 in a sterile glass vial and adding 9.08 mL of QS-21 (1 mg/mL) to the diluted ALF55 while slowly stirring.

Alhydrogel refers to a range of aluminum hydroxide gel products which have been specifically developed for use as an adjuvant in human and veterinary vaccines. The gel is a suspension of boehmite-like (aluminium oxyhydroxide) hydrated nano/micron size crystals in loose aggregates. The products have very low conductivity due to the absence of buffering ions. They have a positive charge at neutral pH and effectively adsorb negatively charged antigens. The primary purpose of the adjuvant in vaccines is to boost the antibody-mediated (Th2) immune response to the antigens. Alhydrogel products can be combined with other adjuvant types (such as monophosphoryl lipids) to achieve a balanced Th1/Th2 immune response. For the purposes of formulating the disclosed vaccines, an alhydrogel stock may be diluted before combining with the disclosed nanoparticles such that the concentration of the aluminum is about 500 μg/ml, about 550 μg/ml, about 600 μg/ml, about 650 μg/ml, about 700 μg/ml, about 750 μg/ml, about 800 μg/ml, about 850 μg/ml, about 900 μg/ml, about 950 μg/ml, about 1000 μg/ml, about 1050 μg/ml, about 1100 μg/ml, about 1150 μg/ml, about 1200 μg/ml, about 1250 μg/ml, about 1300 μg/ml, about 1350 μg/ml, about 1400 μg/ml, about 1450 μg/ml, or about 1500 μg/ml, or more.

Other vaccine adjuvants are known in the art, and based on the results reported herein with respect to ALFQ, and Alhydrogel, those of skill in the art will understand that other adjuvants also could be used with and complement the function of the disclosed antigens and nanoparticles. Other adjuvants that are suitable for use with the disclosed antigens and nanoparticles include, but are not limited to, monophosphoryl lipid A (MPLA), oil in water emulsions, ADJUPLEX™ (a lecithin and carbomer homopolymer), ADDAVAX™ (a squalene-based oil-in-water nano-emulsion), CARBOPOL® polymers (crosslinked polyacrylic acid polymers), Poly IC:LC (a synthetic complex of carboxymethylcellulose, polyinosinic-polycytidylic acid, and poly-L-lysine double-stranded RNA), PolyL:C (polyinosinic:polycytidylic acid), CpG oligodeoxynucleotides, Flagellin, Iscomatrix (comprised of saponin, cholesterol, and dipalmitoylphosphatidylcholine), virosomes, MF59 (a squalene-based oil-in-water emulsion), AS03 (a squalene-based oil-in-water emulsion), and AS04 (alum-absorbed 3-O-desacyl-4′-monophosphoryl lipid A), among others.

### J. Pharmaceutical Compositions

Pharmaceutical compositions of the present disclosure include immunogenic compositions (e.g., vaccines) comprising nanoparticles or mRNA molecules as disclosed herein.

In some embodiments, the pharmaceutical compositions will also comprise an adjuvant (e.g., ALFQ, alhydrogel, or a combination thereof, or an adjuvant suitable for use with an mRNA vaccine). The nanoparticle(s) or mRNA molecule(s), alone or in combination with one or more adjuvants, may be formulated into a suitable carrier to form a pharmaceutical composition suitable for the intended route of administration. It also should be understood that an immunogenic composition as described herein may itself be a pharmaceutical composition, and may comprise, e.g., an adjuvant and/or a suitable carrier for the intended route of administration. Thus, in the discussion that follows, reference to a “pharmaceutical composition” should be understood to encompass embodiments of an immunogenic composition as described herein.

In some embodiments, the pharmaceutical composition is formulated for systemic administration via parenteral delivery. Parenteral administration includes intravenous, intra-arterial, subcutaneous, intradermal, intraperitoneal, or intramuscular injection or infusion. Formulations for parenteral administration may include sterile aqueous solutions, which may also contain buffers, diluents and other pharmaceutically acceptable additives known to the skilled artisan. For intravenous use, the total concentration of solutes may be controlled to render the preparation isotonic. Intravenous, intra-arterial, subcutaneous, or intramuscular injection are preferred routes of administration. Additionally or alternatively, the disclosed vaccines can be formulated for intranasal administration or administration via contact with another mucosa membrane.

Pharmaceutical compositions for injection may be presented in unit dosage form, e.g., in ampules, or in multi-dose containers, optionally with an added preservative. The compositions may take such forms as suspensions, solutions or emulsions in oily or aqueous vehicles, and may contain formulatory agents such as suspending, stabilizing and/or dispersing agents. The disclosed vaccines may be formulated using any suitable pharmaceutically acceptable excipients.

Pharmaceutical compositions for intranasal administration may take the form of liquid dispersions, suspensions, solutions, or emulsions and may be incorporated into a nasal aerosol or nasal spray. Such compositions may contain formulatory agents such as suspending, stabilizing and/or dispersing agents, and may formulated using any suitable pharmaceutically acceptable excipients. Intranasal administration includes administration via the nose, either with or without concomitant inhalation during administration. Such administration is typically through contact of a disclosed vaccine with the nasal mucosa, nasal turbinates, or sinus cavity. Administration by inhalation may comprise intranasal administration, or may include oral inhalation. Such administration may also include contact with the oral mucosa, bronchial mucosa, and other epithelia.

The disclosed immunogenic compositions may be formulated to be administered concurrently with another therapeutic agent. The immunogenic compositions may be formulated to be administered in sequence with another therapeutic agent. For example, the immunogenic compositions may be administered either before or after the subject has received a regimen of an anti-viral therapy.

Any of the immunogenic compositions and pharmaceutical compositions disclosed herein can be used for treating or preventing a coronavirus infection, such as SARS-CoV-2 infection (e.g., COVID-19) or SARS-CoV-1 infection, for example. A pharmaceutical composition or immunogenic composition for use against a specific coronavirus infection (such as SARS-CoV-2), typically will include antigenic peptides of the target coronavirus (e.g., SARS-CoV-2) (or mRNA encoding them), but optionally additionally or alternatively may include antigenic peptides of a closely related coronavirus (such as SARS-CoV-1) (or mRNA encoding them). Optimal doses and routes of administration may vary depending on the nature of the immunogenic composition (e.g., mRNA vs. nanoparticle), virus(es) being targeted, and subject being treated.

### IV. Treatment and Prevention of Coronavirus Infection

The present disclosure provides methods of treatment and prevention of coronavirus infections, including but not limited to sarbecovirus infections and merbecovirus infections, by administering an immunogenic composition (e.g. a vaccine) as described herein, comprising one or more of the nanoparticles or mRNA molecules disclosed herein. The present disclosure also provides uses of the disclosed immunogenic composition and pharmaceutical compositions for treating or preventing coronavirus infections, such as SARS-CoV-2 infections (e.g., COVID-19), SARS infections, and MERS infections. In accordance with any methods and uses disclosed herein, the subject may be at risk of a coronavirus infection or may already be infected with a coronavirus. Additionally or alternatively, the subject may not previously have been administered a vaccine for prevention of a coronavirus infection or may previously have been administered a vaccine for prevention of a coronavirus infection. Methods targeting a specific coronavirus infection (such as SARS-CoV-2), typically will use an immunogenic composition or pharmaceutical composition that includes antigenic peptides of the target coronavirus (e.g., SARS-CoV-2) (or mRNA encoding them), but optionally additionally or alternatively may include antigenic peptides of a closely related coronavirus (such as SARS-CoV-1) (or mRNA encoding them).

The disclosed methods comprise administering to a subject an effective amount of one or more of the immunogenic composition (e.g., vaccines) or pharmaceutical compositions disclosed herein. Administration may be performed via intravenous, intra-arterial, intramuscular, subcutaneous, or intradermal injection. In some embodiments, the subject may be at risk of exposure to a coronavirus, such as SARS-CoV-2, MERS, or SARS-CoV-1, for example. In some embodiments, the subject may have previously been exposed to a coronavirus, such as SARS-CoV-2, MERS, or SARS-CoV-1. In some embodiments, the subject has not previously been administered a vaccine for prevention of a coronavirus infection. In some embodiments the subject previously has been administered a vaccine for prevention of a coronavirus infection. In some embodiments, the subject may have an active infection which may be treated as a result of the administration. In some embodiments, the administration of the vaccine prevents the subject from developing a coronavirus infection. In some embodiments, the method elicits an immune response in the subject against a coronavirus, optionally wherein the immune response comprises neutralizing antibodies, further optionally wherein the neutralizing antibodies cross-neutralizes two or more coronavirus strains, further optionally wherein the neutralizing antibodies cross-neutralizes one or more coronavirus strains that is not a component strain of the immunogenic composition.

The methods can further include administration of a priming agent (i.e., “primer”) for the nanoparticle vaccine or immunogenic composition as described herein. The primer can be administered prior to the administration of the nanoparticle vaccine (e.g., 1 week, 2 weeks, 3 weeks, 4 weeks, 5 weeks, or 6 or more weeks prior). The primer may comprise a nucleic acid (i.e., DNA or mRNA) that encodes a fusion protein or all, a fragment, or a variant of the RBD of a coronavirus S protein (e.g., the S protein of SARS-CoV-2 or SARS-CoV-1).

For the purposes of the disclosed methods and uses, treatment and/or prevention of infection by all coronaviruses, are specifically contemplated, including treatment and/or prevention of various strains of SARS-CoV-2. Also contemplated are methods and uses for treatment and/or prevention of infection by all strains and variants of SARS-CoV-1, SARS-CoV-2, and MERS-CoV, as well as all strains and variants of other coronaviruses disclosed herein.

Dosage regimens can be adjusted to provide the optimum desired response (e.g., production of antibodies and/or cytokines against a coronavirus). For example, in some embodiments, a single bolus of vaccine (e.g., an immunogenic composition as described herein) may be administered, while in some embodiments, several doses may be administered over time, or the dose may be proportionally reduced or increased as indicated by the situation. For example, in some embodiments the disclosed vaccines may be administered once or twice weekly, once or twice monthly, once every week, once every other week, once every three weeks, once every four weeks, once every other month, once every three months, once every four months, once every five months, once every six months, once every seven weeks, once every eight weeks, once every three months, once every four months, once every five months, once every six months, or once a year. In some embodiments, a subject may be administered an initial dose and then receive one or more booster doses with a predefined span of time in between each dose (e.g., 1, 2, 3, or 4 weeks, or 1, 2, 3, 4, 5, 6, 9, or 12 months). In some embodiments, a subject may receive only a single dose. In some embodiments, a subject may receive an initial dose followed by one or more subsequent doses of an equal or lesser concentration at a set time after this initial dose, such as 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 or more weeks, such as 24 weeks, 52 weeks, 104 weeks, 260 weeks, or 520 weeks.

Doses may likewise by adjusted to provide the optimum desired response. For example, in some embodiments, a dose of the disclosed vaccines may comprise 1 μg to 50 mg of vaccine. A single does may comprise about 1 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg about 70 μg, about 75 μg, about 80 μg, about 85 μg, about 90 μg, about 95 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 100 μg, about 825 μg, about 850 μg, about 875 μg, about 900 μg, about 925 μg, about 950 μg, about 975 μg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 5.75 mg, about 5 mg, about 10 mg, about 15 mg, about 20 mg, about 25 mg, about 30 mg, about 35 mg, about 45 mg, or about 50 mg. In some embodiments, a single dose may comprise 4 mg or less of the vaccine or nanoparticle.

Alternatively, dosing may be based on the number of nanoparticles administered to a subject. For example, in some embodiments, a dose of the disclosed vaccines may comprise 1.0×10⁸ to 1.0×10¹² nanoparticles. For example, a single dose may comprise 1.0×10⁸, 1.5×10⁸, 2.0×10⁸, 2.5×10⁸, 3.0×10⁸, 3.5×10⁸, 4.0×10⁸, 4.5×10⁸, 5.0×10⁸, 5.5×10⁸, 6.0×10⁸, 6.5×10⁸, 7.0×10⁸, 7.5×10⁸, 8.0×10⁸, 8.5×10⁸, 9.0×10⁸, 9.5×10⁸, 1.0×10⁹, 1.5×10⁹, 2.0×10⁹, 2.5×10⁹, 3.0×10⁹, 3.5×10⁹, 4.0×10⁹, 4.5×10⁹, 5.0×10⁹, 5.5×10⁹, 6.0×10⁹, 6.5×10⁹, 7.0×10⁹, 7.5×10⁹, 8.0×10⁹, 8.5×10⁹, 9.0×10⁹, 9.5×10⁹, 10×10¹⁰, 1.5×10¹⁰, 2.0×10¹⁰, 2.5×10¹⁰, 3.0×10¹⁰, 3.5×10¹⁰, 4.0×10¹⁰, 4.5×10¹⁰, 5.0×10¹⁰, 5.5×10¹⁰, 6.0×10¹⁰, 6.5×10¹⁰, 7.0×10¹⁰, 7.5×10¹⁰, 8.0×10¹⁰, 8.5×10¹⁰, 9.0×10¹⁰, 9.5×10¹⁰, 1.0×10¹¹, 1.5×10¹¹, 2.0×10¹¹, 2.5×10¹¹, 3.0×10¹¹, 3.5×10¹¹, 4.0×10¹¹, 4.5×10¹¹, 5.0×10¹¹, 5.5×10¹¹, 6.0×10¹¹, 6.5×10¹¹, 7.0×10¹¹, 7.5×10¹¹, 8.0×10¹¹, 8.5×10¹¹, 9.0×10¹¹, 9.5×10¹¹, or 1.0×10¹² nanoparticles. In some embodiments, the dose may be about 9.5×10⁸, about 9.75×10⁸, about 9.85×10⁸, about 9.95×10⁸, about 1.0×10⁹, about 1.1×10⁹, about 1.15×10⁹, about 1.2×10⁹, about 1.25×10⁹, about 1.3×10⁹, about 1.35×10⁹, about 1.4×10⁹, about 1.45×10⁹, or about 1.5×10⁹ nanoparticles

In some embodiments, the subject is a mammal. In some embodiments, the subject is a human. In embodiments in which the subject is a human, the subject may be at least 18 years old, 40 years old, at least 45 years old, at least 50 years old, at least 55 years old, at least 60 years old, at least 65 years old, at least 70 years old, at least 75 years old, or at least 80 years old or older. In some embodiments, the subject is a pediatric subject (i.e., less than 18 years old).

### V. Screening for Binding Molecules

In addition to being used for treatment, the disclosed nanoparticles and fusion proteins can be used to screen binding molecules, such as antibodies, for ability to bind to and neutralize a coronavirus (e.g., SARS-CoV-1 or SARS-CoV-2). Any of the fusion proteins disclosed herein can be contacted with a putative coronavirus binding molecule, such as a putative anti-coronavirus antibody, and assessed for binding to the fusion protein or nanoparticle. Antibodies (or other binding molecules) that bind to the fusion proteins disclosed herein are expected to be neutralizing.

### VI. Passive Immunotherapy and Treatment with Binding Molecules

Binding molecules (e.g., antibodies that bind to SARS-CoV-2 or another coronavirus as disclosed herein) can be used for passive immunotherapy to prevent the development of a coronavirus infection or for the treatment of a subject that already has a coronavirus infection. In general, coronavirus-specific antibodies can be obtained from a subject that was administered an immunogenic composition as disclosed herein or coronavirus-specific antibodies can be identified from a subject that recovered from a coronavirus infection (e.g., COVID-19) using the disclosed fusion proteins and nanoparticles as bait for a screening assay. These antibodies can be administered to a subject that has been exposed to or is at risk of exposure to a coronavirus in order to prevent the development of a coronavirus infection such as COVID-19 or SARS-CoV-1 infection, for example (i.e., the antibodies can serve as a “passive immunotherapy”). Additionally or alternatively, these antibodies can be administered to a subject that has been infected with a coronavirus, such as SARS-CoV-1 or SARS-CoV-2, to treat the infection by, for example, reducing or eliminating viral load.

The disclosed binding proteins may be or be derived from a human IgG1 antibody, a human IgG2 antibody, a human IgG3 antibody, or a human IgG4 antibody. In some embodiments, the binding protein may be or be derived from a class of antibody selected from IgG, IgM, IgA, IgE, and IgD. That is, the disclosed binding proteins may comprise all or part of the constant regions, framework regions, or a combination thereof of an IgG, IgM, IgA, IgE, or IgD antibody. For instance, a disclosed binding protein comprising an IgG1 immunoglobulin structure may be modified to replace (or “switch”) the IgG1 structure with the corresponding structure of another IgG-class immunoglobulin or an IgM, IgA, IgE, or IgD immunoglobulin. This type of modification or switching may be performed in order to augment the neutralization functions of the peptide, such as antibody dependent cell cytotoxicity (ADCC) and complement fixation (CDC). A person of ordinary skill in the art will understand that, for example, a recombinant IgG1 immunoglobulin structure can be “switched” to the corresponding regions of immunoglobulin structures from other immunoglobulin classes, such as recombinant secretory IgAQ1 or recombinant secretory IgA2, such as may be useful for topical application onto mucosal surfaces. For example, immunoglobulin IgA structures are known to have applications in protective immune surveillance directed against invasion of infectious diseases, which makes such structures suitable for methods of using the disclosed binding proteins in such contexts, e.g., treating or preventing coronavirus infection (e.g., COVID-19 or SARS-CoV-1 infection) or the spread of coronavirus from one individual to another.

Any of the coronavirus-specific binding proteins or antibodies obtained from a subject inoculated with a disclosed immunogenic composition or screened/selected using the disclosed fusion proteins can be used for treating and/or preventing a coronavirus infection, such as COVID-19 or SARS-CoV-1 infection, for example. Optimal doses and routes of administration may vary, such as based on the route of administration and dosage form, the age and weight of the subject, and/or the subject's condition, including the type and severity of the coronavirus infection, and can be determined by the skilled practitioner. The binding proteins can be formulated in a pharmaceutical composition suitable for administration to a subject by any intended route of administration.

### VII. Example Embodiments

The present disclosure provides the following example embodiments, which are non-limiting with respect to the disclosure.

Embodiment 1: A nanoparticle comprising a fusion protein comprising a nanoparticle-forming peptide and at least two antigenic coronavirus peptides selected from: a receptor-binding domain (RBD) of a coronavirus, or a fragment or variant thereof, an N-terminal domain (NTD) of a coronavirus, or a fragment or variant thereof, an S1 domain of a coronavirus, or a fragment or variant thereof, a stabilized extracellular spike S-2P domain of a coronavirus, or a fragment or variant thereof, a stabilized extracellular spike S domain of a coronavirus, or a fragment or variant thereof, a stabilized extracellular spike S-trimer of a coronavirus, or a fragment or variant thereof, and a mosaic coronavirus spike protein, wherein at least one domain of the mosaic coronavirus spike protein is substituted or added from a heterologous.

Embodiment 2: A nanoparticle of embodiment 1, wherein the nanoparticle-forming peptide comprises or is a ferritin protein or a fragment or variant thereof.

Embodiment 3: A nanoparticle of embodiment 1 or 2, wherein the nanoparticle-forming peptide comprises or is Helicobacter pylori ferritin (Hpf) or a fragment or variant thereof.

Embodiment 4: A nanoparticle of any one of embodiments 1-3, wherein the nanoparticle-forming peptide comprises an amino acid sequence selected from:
a. ESQVRQQFSKDIEKLLNEQVNKEMQSSNLYMSMSSWCYTHSLDGAGLFL FDHAAEEYEHAKKLIIFLNENNVPVQLTSISAPEHKFEGLTQIFQKAYEHE QHISESINNIVDHAIKSKDHATFNFLQWYVAEQHEEEVLFKDILDKIELIGN ENHGLYLADQYVKGIAKSRKSGS (SEQ ID NO: 1) or a fragment or variant thereof,
b. DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINNIVD HAIKSKDHATFNFLQWYVAEQHEEEVLFKDILDKIELIGNENHGLYLADQ YVKGIAKSRKSGS (SEQ ID NO: 2) or a fragment or variant thereof, and
c. SKDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESINNI VDHAIKSKDHATFNFLQWYVAEQHEEEVLFKDILDKIELIGNENHGLYLA DQYVKGIAKSRKSGS (SEQ ID NO: 3) or a fragment or variant thereof.

Embodiment 5: A nanoparticle of any one of embodiments 1-4, wherein the at least two antigenic coronavirus peptides are connected via a linker.

Embodiment 6: A nanoparticle of any one of embodiments 1-5, wherein the at least two antigenic coronavirus peptides are connected to the nanoparticle-forming peptide via a linker.

Embodiment 7: A nanoparticle of embodiment 5 or 6, wherein the linker comprises an amino acid sequence selected from: GSGGGG (SEQ ID NO: 11), GGGG (SEQ ID NO: 15), GSGG (SEQ ID NO: 5), GGG (SEQ ID NO: 16), and SGG (SEQ ID NO: 17).

Embodiment 8: A nanoparticle of any one of embodiments 1-7, wherein the fusion protein comprises 3-10 antigenic coronavirus peptides connected in series, optionally wherein the antigenic coronavirus peptides are connected via peptide linkers.

Embodiment 9: A nanoparticle of any one of embodiments 1-8, wherein the at least two antigenic coronavirus peptides are isolated or derived from one or more coronaviruses selected from SARS-CoV-2, human coronavirus OC43 (hCoV-OC43), Middle East respiratory syndrome-related coronavirus (MERS-CoV), severe acute respiratory syndrome-related coronavirus (SARS-CoV-1), HKU-1, 229E, or NL63.

Embodiment 10: A nanoparticle of any one of embodiments 1-9, wherein the fusion protein comprises a format selected from beads on a string, domain fusion, loop insertion, or domain insertion.

Embodiment 11: A nanoparticle of any one of embodiments 1-10, wherein the fusion protein comprises a format shown in FIG. 2.

Embodiment 12: A nanoparticle of any one of embodiments 1-11, wherein the fusion protein comprises an amino acid sequence disclosed in Table 6.

Embodiment 13: A vaccine comprising a nanoparticle of any one of embodiments 1-12.

Embodiment 14: A vaccine of embodiment 13, wherein the vaccine further comprises one or more adjuvants selected from ALFQ, alhydrogel, and combinations thereof.

Embodiment 15: A messenger RNA (mRNA) encoding a nanoparticle according to any one of embodiments 1-12.

Embodiment 16: A method of treating or preventing a coronavirus infection in a subject in need thereof, comprising administering to a subject in need thereof the nanoparticle according to any one of embodiments 1-12, the vaccine according to any one of embodiments 13-14 or the mRNA according to embodiment 15.

Embodiment 17: A method of embodiment 16, wherein the subject is at risk of contracting a coronavirus infection.

Embodiment 18: A method of embodiment 16, wherein the subject has already contracted a coronavirus infection.

Embodiment 19: A nanoparticle according to any one of embodiments 1-12, a vaccine according to any one of embodiments 13-14, or a mRNA according to embodiment 15, for use in treating or preventing a coronavirus infection in a subject in need thereof.

Embodiment 20: A nanoparticle, vaccine or mRNA for use of embodiment 19, wherein the subject is at risk of contracting a coronavirus infection.

Embodiment 21: A nanoparticle, vaccine, or mRNA for use of embodiment 19, wherein the subject has already contracted a coronavirus infection.

Embodiment 22: Use of a nanoparticle according to any one of embodiments 1-12, a vaccine according to any of embodiments 13-14, or the mRNA according to embodiment 16 in the preparation of a medicament for treating or preventing a coronavirus infection in a subject in need thereof.

Embodiment 23: A DNA molecule, comprising a sequence encoding a nanoparticle according to any one of embodiments 1-12.

Embodiment 24: A plasmid comprising the DNA molecule of embodiment 23.

Embodiment 25: A plasmid of embodiment 24, wherein the plasmid can express the DNA molecule in vivo.

The following examples are given to illustrate the present disclosure. It should be understood that the invention is not to be limited to the specific conditions or details described in these examples.

### EXAMPLES

### Example 1—Screening of Disclosed Fusion Proteins

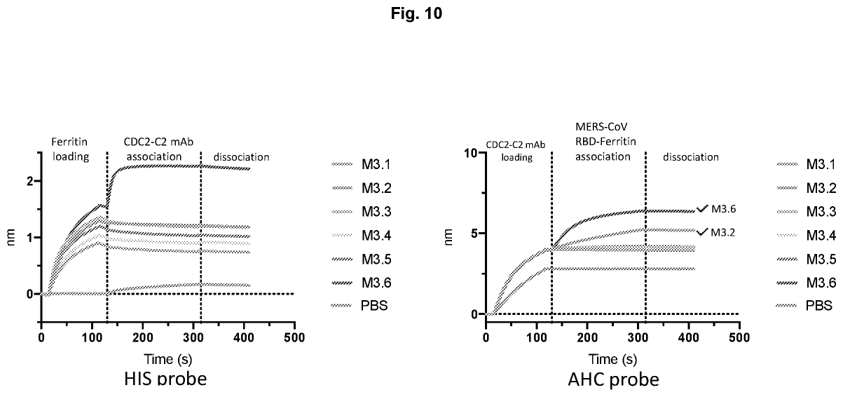

Binding studies of MERS-CoV RBD-ferritin nanoparticle immunogens were performed, assessing MERS-CoV RBD-ferritin constructs for binding to MERS-CoV-neutralizing human monoclonal antibody CDC-C2 in two formats. Briefly, biosensors are hydrated in PBS prior to use. Assay steps are performed at 30° C. with agitation set at 1,000 crpm in the Octet RED96 instrument (ForteBio). Biosensors are equilibrated in assay buffer (PBS) for 15 seconds before loading of IgG antibodies (30 g/ml diluted in PBS). MERS-CoV neutralizing antibodies that target the spike RBD used include D12, F11, CDC2-C2, and JC57-11, while SARS-CoV-2 neutralizing antibodies include WRAIR-2125, WRAIR-5001, and ShAb02. MERS-CoV and SARS-CoV-2 antibodies were immobilized onto AHC biosensors (ForteBio) for 100 seconds, followed by a brief baseline in assay buffer for 15 sec. Immobilized antibodies were then dipped in various antigens for 180 seconds. Response values were measured at the end of the association step. Results for constructs M.1-M3.6 are shown in FIG. 10.

Similar antigenic characterization of MVERS RBD-containing immunogens M3.7, M3.12 and M3.13, and dual-RBD constructs SARS-CoV-2-ERS-CoV RR-FN immunogens 248-250 and 267-270 was conducted using a set of neutralizing antibodies by octet biolayer interferometry. The results (binding responses after 180 seconds of association) are shown in Table 4 below.

TABLE 4

| | M3.7 | M3.12 | M3.13 | CoV267 | CoV268 | CoV269 | CoV270 | CoV247 | CoV248 | CoV249 | CoV250 |
|---|---|---|---|---|---|---|---|---|---|---|---|
| mAbs | | | | | | | | | | | |
| MERS-CoV | | | | | | | | | | | |
| specific | | | | | | | | | | | |
| D12 | 1.03 | 0.93 | 1.13 | 1.45 | 1.24 | 1.43 | 1.33 | 1.37 | 1.08 | 1.41 | 1.19 |
| F11 | 0.83 | 0.779 | 0.90 | 1.15 | 0.98 | 1.09 | 1.03 | 1.22 | 0.87 | 1.30 | 1.08 |
| CDC2-C2 | 1.35 | 1.66 | 1.62 | 2.03 | 1.92 | 1.94 | 1.90 | 2.05 | 1.69 | 2.18 | 2.05 |
| JC57-11 | 0.85 | 0.90 | 1.13 | 1.28 | 1.23 | 1.32 | 1.20 | 1.39 | 1.07 | 1.45 | 1.35 |
| SARS-CoV- | | | | | | | | | | | |
| 2 specific | | | | | | | | | | | |
| WRAIR- | −0.03 | −0.01 | 0.003 | 1.76 | 1.85 | 1.75 | 1.76 | 1.80 | 1.61 | 1.95 | 1.83 |
| 2125 | | | | | | | | | | | |
| WRAIR- | −0.04 | 0.013 | −0.03 | 1.77 | 1.83 | 1.78 | 1.73 | 1.79 | 1.29 | 1.98 | 1.82 |
| 5001 | | | | | | | | | | | |
| ShAb02 | −0.04 | 0.03 | 0.01 | 2.30 | 2.27 | 2.16 | 2.15 | 2.60 | 2.10 | 2.57 | 2.28 |

Values are response (nm) after 180 seconds.

Antigenic characterization of SARS-CoV-1 RBD-containing immunogens CoV263, CoV277, CoV278, CoV316, and CoV317 immunogens were assessed using a set of neutralizing antibodies by octet biolayer interferometry. The results are shown in Table 5 below.

TABLE 5

| | | | | | | | | | | CoV1B06-PL |
|---|---|---|---|---|---|---|---|---|---|---|
| | | | | | | | | | | (positive |
| | CoV263 | CoV277 | CoV278 | CoV316 | CoV317 | CoV272 | CoV239 | CoV256 | CoVS01 | ctrl.) |
| mAbs | | | | | | | | | | |
| hACE2-Fc | 0.40 | 0.40 | 0.48 | 0.65 | 0.91 | 0.40 | 0.18 | 0.47 | 0.24 | 0.36 |
| (CoV225) | | | | | | | | | | |
| ShAb01 | 1.07 | 0.82 | 0.68 | 0.83 | 1.09 | −0.04 | 0.90 | 0.98 | 0.06 | 0.61 |
| CR3022 | 0.98 | 1.10 | 1.31 | 1.25 | 1.03 | −0.03 | 0.42 | 1.42 | 0.13 | 0.67 |
| SARS- | | | | | | | | | | |
| CoV-2 | | | | | | | | | | |
| specific | | | | | | | | | | |
| ShAb02 | 2.03 | 1.57 | 1.28 | 1.25 | 1.66 | 0.79 | 0.73 | 1.85 | −0.04 | 1.61 |

Values are response (nm) after 180 seconds.

### Example 2—Production and Size-Estimation of Fusion Proteins

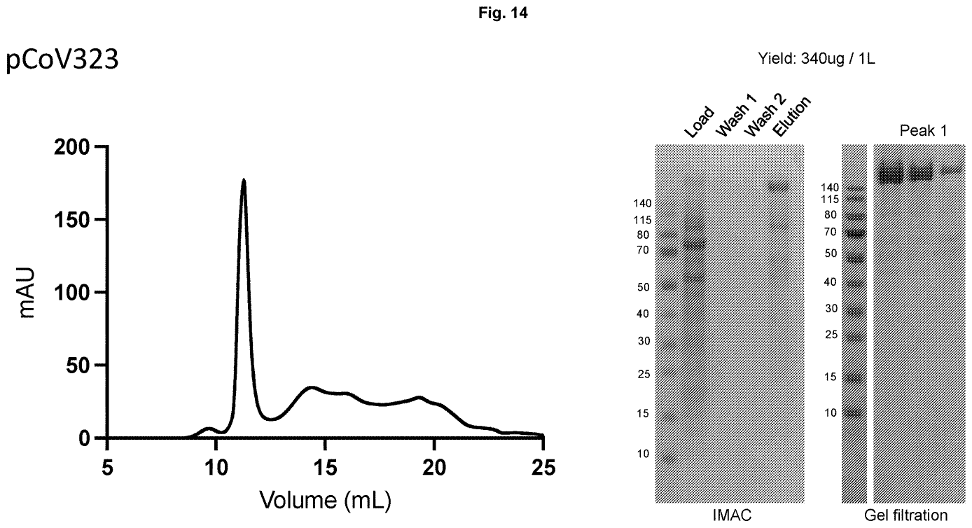

RR-SpFN constructs were designed and tested for expression, yield, and nanoparticle formation. Construct pCoV323 (RR-SpFN MR14-SARS1-SpFN) was expressed in Expi293F cells for 5 days at 37° C. and purified by Galanthus nivalis lectin (GNA) affinity chromatography. This construct showed reasonable expression levels of 0.4 mg/L medium supernatant. Purified protein was assessed by SDS-PAGE and size-exclusion chromatography to assess expression and to evaluate size. The results are shown in FIG. 14, with the RR-SpFN construct showing appropriate size by SDS-PAGE and nanoparticle formation by size-exclusion chromatography.

### Example 3 mRNA Construct Expression

mRNA constructs encoding spike antigens were provided and expressed in HeLa cells. Additionally, expressed fusion proteins were assessed using a set of neutralizing antibodies by octet biolayer interferometry.

### Methods

HeLa cells were plated in 24-well plates at 0.075 million cells/well in 0.5 mL EMEM+10% FBS. Cells were transfected the next day with 1 μg/million cells mRNA constructs with lipofectamine 2000. The different mRNA constructs have been tested for in vitro expression and secretion in HeLa cells after transfection. 24 hours post-transfection, the cell lysates and supernatants (to assess secretion) were analyzed in dot blot by probing the Spikes or RBDs with anti-RBD monoclonal antibodies (mAbs) targeting conformational neutralizing epitopes (SA55 for example) and/or with anti-ferritin mAb.

For octet binding studies, approximately 12 μg of mRNA in total was used to transfect 25 ml of Expi 293F cells. Supernatant was harvested after 2-4 days and the supernatant was assessed for binding against a set of CoV-specific antibodies, human ACE2, and a negative control influenza antibody by biolayer interferometry.

### Preparation of Lysates

Cells were harvested the 22-24 hours later and lysed in 225 μL per well of CelLytic M+1× HALT. Lysates were incubated on ice for 10 minutes then cleared in a microcentrifuge at max speed for 10 min at 4° C. Lysates were diluted 2-fold and 4-fold with PBS for downstream analysis.

### Preparation of Supernatant Samples

Cell culture supernatants were harvested the 22-24 hours after transfection. Supernatant samples were diluted 2-fold and 4-fold with PBS for downstream analysis.

### Expression Analysis

Neat, 2-fold, and 4-fold diluted lysate or supernatant samples were spotted onto a dry nitrocellulose membrane in 1 μL dots. The membranes were then allowed to dry completely before further handling. Blots were blocked with Intercept blocking buffer for 1 hour at room temperature. Blots were stained with human anti-Spike antibody or human anti-ferritin antibody. Antibody staining was done in Intercept Blocking buffer+0.2% Tween 20 for overnight at 4° C. Blots were washed with TBST 3×5 minutes. Blots were then stained with donkey anti-human IR800 secondary antibody in Intercept Blocking buffer+0.2% Tween 20 for 1 hr at RT. Blots were washed with TBST 4×5 minutes and scanned on a Licor odyssey.

### Expression Results

The following constructs were expressed and detected in lysate and/or supernatant, as indicated in Table 10. Expression in the supernatant indicated that the protein was secreted. All mRNA expressed the expected antigens in cell lysates; the S-2P antigens were not detected in the supernatant, as expected for transmembrane proteins; some of the more complex multi-strain constructs (R-SpFN and RR-SpFN) were not detected in the supernatant, indicating either low or defective secretion. The results are summarized in Table 10 below.

TABLE 10

| | | Lysate | Supernatant |
|---|---|---|---|
| SEQ ID NO | Construct Name | Expression | Expression |
| 553 | WA-1 RFN | Yes | Yes |
| 554 | Beta RFN | Yes | Yes |
| 555 | BQ1.1 RFN | Yes | Yes |
| 556 | SARS-CoV-1 RFN | Yes | Yes |
| 557 | Khosta-2 RFN | Yes | Yes |
| 558 | BANAL20-247 RFN | Yes | Yes |
| 559 | MERS RFN | Yes | Yes |
| 560 | WA-1 SpFN | Yes | Yes |
| 561 | Beta SpFN | Yes | Yes |
| 562 | BQ1.1 SpFN | Yes | Yes |
| 563 | SARS-CoV-1 SpFN | Yes | Yes |
| 564 | Khosta-2 SpFN | Yes | Yes |
| 565 | MERS SpFN | Yes | Yes |
| 566 | BANAL20-247 SpFN | Yes | Yes |
| 567 | WA1 mRNA_S2P | Yes | No |
| 568 | Beta mRNA_S2P | Yes | No |
| 569 | BQ1.1 mRNA_S2P | Yes | No |
| 570 | SARS mRNA_S2P | Yes | No |
| 571 | Khosta2 mRNA_S2P | Yes | No |
| 572 | BANAL20247 mRNA_S2P | Yes | No |
| 573 | MERS mRNA_S2P | Yes | No |
| 574 | XBB.1.5 mRNA_S2P | N/A | N/A |
| 575 | Khosta2-WA1 RRFN | Yes | Yes |
| 576 | WA1-Khosta2 RRFN | Yes | Yes |
| 577 | SARS1-WA1 RRFN | Yes | Yes |
| 578 | WA1-SARSI RRFN | Yes | Yes |
| N/A | Beta-WA1 RRFN | Yes | Yes |
| N/A | WA1-Beta RRFN | Yes | Yes |
| N/A | BA4/5-WA1 RRFN | Yes | Yes |
| N/A | WA1-BA4/5 RRFN | Yes | Yes |
| N/A | BANAL20247-WA1 RRFN | Yes | Yes |
| N/A | WA1-BANAL20247 RRFN | Yes | Yes |
| N/A | MERS-WA1 RRFN - pCoV249-like | Yes | Yes |
| N/A | WA1-MERS RRFN - pCoV247-like | Yes | Yes |
| N/A | BQ1.1-WA1 RRFN | Yes | Yes |
| N/A | Beta-WA1 R-SpFN | Yes | No |
| N/A | BA4/5-WA1 R-SpFN | Yes | No |
| N/A | SARS1-WAI R-spFN | Yes | No |
| N/A | MERS-WA1 R-SpFN | Yes | No |
| N/A | Khosta2-WA1 R-spFN | Yes | Yes |
| N/A | BQ1.1-WA1 R-SpFN | Yes | No |
| 579 | WA1-BANAL20247-SARS1-WA1 RR- | Yes | Yes |
| | mosSpFN | | |
| 580 | SARS1-BANAL20247-Khosta2-WA1 RR- | Yes | No |
| | mosSpFN | | |
| 581 | Khosta2-BANAL20247-SARS1-WA1 RR- | Yes | Yes |
| | mosSpFN | | |
| N/A | WA1-SARS1-BANAL20247-WA1 RR- | Yes | No |
| | mosSpFN | | |
| N/A | SARS1-BANAL20247-WA1 RR-mosSpFN | Yes | No |
| 582 | XBB.1.5 SpFN | N/A | N/A |

The specific constructs for which SEQ ID NOs are not provided were not moved forward for in vivo studies at the present time.

### Octet Binding Results

The results of the octet binding studies are summarized in Table 11 below.

TABLE 11

| | Antibody or human ACE2 receptor binding | | | | | | |
|---|---|---|---|---|---|---|---|
| | | | | | ShAb34 | | Flu-1 |
| | | | WRAIR- | | (MERS- | | (negative |
| Construct Name | ShAb02 | ShAb01 | 2173 | ShAb24 | specific) | ACE2-Fc | control) |
| CO1-WA1 RFN - | 2.345 | 2.244 | 1.475 | 2.294 | 0 | 1.233 | 0 |
| pCoV131 | | | | | | | |
| CO2 - WA-1 RFN - | 2.546 | 2.482 | 1.597 | 2.379 | 0 | 0.914 | 0 |
| pCoV131 | | | | | | | |
| CO1 - Beta RFN | 1.245 | 0.929 | 0.478 | 1.131 | 0 | 0.247 | 0 |
| CO2 - Beta RFN | 1.440 | 0.939 | 0.441 | 1.063 | 0 | 0.242 | 0 |
| CO1 - SARS-CoV-1 | 0.120 | 0.782 | 0.282 | 0.793 | 0 | 0.447 | 0 |
| RFN | | | | | | | |
| CO2 - SARS-CoV-1 | 0.111 | 0.768 | 0.269 | 0.788 | 0 | 0.380 | 0 |
| RFN | | | | | | | |
| CO1 - empty FN | 0.101 | 0.077 | 0.275 | 0.048 | 0 | 0.107 | 0 |
| CO2 - empty FN | 0.382 | 0.291 | 0.629 | 0.484 | 0 | 0.063 | 0 |
| CO2 - Khosta-2 RFN | 1.851 | 1.578 | 0.950 | 1.727 | 0 | 0.912 | 0 |
| CO1 - BANAL20-247 | 2.041 | 1.537 | 0.816 | 1.397 | 0 | 0.224 | 0 |
| RFN | | | | | | | |
| CO2 - BANAL20-247 | 2.087 | 1.629 | 0.335 | 1.498 | 0 | 0.084 | 0 |
| RFN | | | | | | | |
| CO1 - MERS RFN - | 0.389 | 0.192 | 0.666 | 0.246 | 1.684 | 0.180 | 0 |
| M3.7-like | | | | | | | |
| CO2 - MERS RFN - | 0.150 | 0.125 | 0.302 | 0.173 | 1.650 | 0.141 | 0 |
| M3.7-like | | | | | | | |
| CO1-WA1 SpFN | 0.957 | 0.323 | 0.908 | 0.017 | 0 | 0.224 | 0 |
| CO2 - WA1 SpFN | 0.772 | 0.259 | 0.897 | 0.315 | 0 | 0.234 | 0 |
| CO2-Beta SpFN | 0.929 | 0.423 | 0.899 | 0.009 | 0 | 0.483 | 0 |
| CO2 - SARS-CoV-1 | 0.070 | 0.100 | 0.190 | 0.042 | 0 | 0.094 | 0 |
| SpFN | | | | | | | |
| CO1 - Khosta-2 SpFN | −0.038 | 0.382 | 0.029 | 0.430 | 0 | 0.047 | 0 |
| CO2 - Khosta-2 SpFN | 0.044 | 0.558 | 0.160 | 0.414 | 0 | 0.356 | 0 |
| CO1 - MERS SpFN | 0.033 | 0.077 | 0.132 | 0.020 | 0.642 | 0.072 | 0 |
| CO2 - MERS SpFN | −0.060 | 0.053 | 0.175 | −0.003 | 1.047 | 0.081 | 0 |
| CO1 - BANAL20-247 | 1.019 | 0.345 | 0.265 | 0.238 | 0 | 0.096 | 0 |
| SpFN | | | | | | | |
| CO2 - BANAL20-247 | 1.079 | 0.604 | 0.234 | 0.457 | 0 | 0.024 | 0 |
| SpFN | | | | | | | |
| CO1 - Beta-WA1 | 1.350 | 1.478 | 1.344 | 1.421 | 0 | 0.272 | 0 |
| RRFN | | | | | | | |
| CO1 - WA1-Beta | 1.175 | 1.064 | 1.129 | 1.339 | 0 | 0.310 | 0 |
| RRFN | | | | | | | |
| CO2 - WA1-Beta | 1.346 | 1.463 | 1.375 | 1.517 | 0 | 0.401 | 0 |
| RRFN | | | | | | | |
| CO2 - BA4/5-WA1 | 1.576 | 1.376 | 1.417 | 1.445 | 0 | 0.347 | 0 |
| RRFN | | | | | | | |
| CO1 - WA1-BA4/5 | 1.028 | 0.457 | 1.143 | 0.761 | 0 | 0.218 | 0 |
| RRFN | | | | | | | |
| CO2 - WA1-BA4/5 | 1.011 | 0.658 | 1.308 | 1.120 | 0 | 0.259 | 0 |
| RRFN | | | | | | | |
| CO2-MERS-WA1 | 1.454 | 1.277 | 1.047 | 1.043 | 1.488 | 0.260 | 0 |
| RRFN -pCoV249-like | | | | | | | |
| CO1 - WA1-MERS | 1.377 | 0.864 | 1.123 | 1.059 | 1.144 | 0.381 | 0 |
| RRFN - pCoV247- | | | | | | | |
| like | | | | | | | |
| CO2 - WA1-MERS | 1.305 | 0.896 | 1.140 | 1.075 | 1.195 | 0.382 | 0 |
| RRFN - pCoV247- | | | | | | | |
| like | | | | | | | |
| CO1 - Khosta2-WA1 | 1.829 | 1.579 | 1.369 | 1.207 | 0 | 0.558 | 0 |
| RRFN | | | | | | | |
| CO2 - Khosta2-WA1 | 1.956 | 1.662 | 1.429 | 1.325 | 0 | 0.519 | 0 |
| RRFN | | | | | | | |
| CO2 - WA1-Khosta2 | 1.653 | 1.467 | 1.350 | 1.257 | 0 | 0.490 | 0 |
| RRFN | | | | | | | |
| CO1 - WA1- | 1.299 | 1.152 | 1.252 | 1.065 | 0 | 0.471 | 0 |
| BANAL20247 RRFN | | | | | | | |
| CO2 - WA1- | 1.350 | 1.339 | 1.233 | 1.189 | 0 | 0.360 | 0 |
| BANAL20247 RRFN | | | | | | | |
| CO2 - Beta-WA1 R- | 0.062 | 0.009 | 0.179 | 0.013 | 0 | 0.063 | 0 |
| SpFN | | | | | | | |
| CO1 - BA4 - 5-WA1 | 0.135 | 0.081 | 0.279 | 0.014 | 0 | 0.064 | 0 |
| R-SpFN | | | | | | | |
| CO1-SARS1-WA-1 | 0.125 | 0.079 | 0.245 | 0.009 | 0 | 0.105 | 0 |
| R-SpFN | | | | | | | |
| CO1 - MERS-WA1 | 0.155 | −0.012 | 0.333 | 0.041 | 0.162 | 0.089 | 0 |
| R-SpFN | | | | | | | |
| CO2 - MERS-WA1 | −0.077 | −0.018 | 0.109 | −0.068 | 0.013 | 0.030 | 0 |
| R-SpFN | | | | | | | |
| CO1 - Khosta2-WA1 | 0.208 | 0.048 | 0.354 | 0.151 | 0 | 0.167 | 0 |
| R-SpFN | | | | | | | |
| CO2 - Khosta2-WA1 | 0.225 | 0.064 | 0.307 | 0.133 | 0 | 0.157 | 0 |
| R-SpFN | | | | | | | |
| CO2-Khosta2- | 0.885 | 0.603 | 0.241 | 0.489 | 0 | 0.337 | 0 |
| BANAL20247- | | | | | | | |
| SARS1-WA1 RR- | | | | | | | |
| mosSpFN | | | | | | | |
| CO1 - SARS1- | 0.194 | 0.109 | 0.160 | 0.132 | 0 | 0.134 | 0 |
| BANAL20247- | | | | | | | |
| Khosta2-WA1 RR- | | | | | | | |
| mosSpFN | | | | | | | |
| CO2-SARS1- | 0.256 | 0.080 | 0.252 | 0.166 | 0 | 0.256 | 0 |
| BANAL20247- | | | | | | | |
| Khosta2-WA1 RR- | | | | | | | |
| mosSpFn | | | | | | | |
| CO1 - SARS1- | 0.068 | 0.024 | 0.148 | −0.004 | 0 | 0.106 | 0 |
| BANAL20247-WA1 | | | | | | | |
| RR-SpFN | | | | | | | |
| CO1-WA1- | 1.266 | 0.604 | 1.041 | 0.690 | 0 | 0.521 | 0 |
| BANAL20247- | | | | | | | |
| SARS1-WA1 RR- | | | | | | | |
| mosSpFN | | | | | | | |
| CO1 - BQ1.1 RFN | 0.465 | 0.102 | 0.168 | 0.438 | 0 | 0.226 | 0 |
| CO2 - BQ1.1 RFN | 0.908 | 0.143 | 0.153 | 0.637 | 0 | 0.236 | 0 |
| CO1 - BQ1.1 SpFN | 0.123 | 0.023 | 0.011 | −0.063 | 0 | 0.080 | 0 |
| CO2 - BQ1.1 SpFN | 0.287 | 0.057 | 0.082 | −0.018 | 0 | 0.158 | 0 |
| CO1 - BQ1.1-WA1 | 1.590 | 0.966 | 1.398 | 1.388 | 0 | 0.342 | 0 |
| RRFN | | | | | | | |
| CO2 - BQ1.1-WA1 | 1.426 | 0.640 | 1.252 | 1.147 | 0 | 0.325 | 0 |
| RRFN | | | | | | | |
| CO1 - BQ1.1-WA1 | 0.183 | 0.074 | 0.289 | −0.003 | 0 | 0.143 | 0 |
| R-SpFN | | | | | | | |
| CO2 - BQ1.1-WA1 | 0.430 | 0.218 | 0.496 | 0.197 | 0 | 0.237 | 0 |
| R-SpFN | | | | | | | |
| CO2 - SARS1-WA1 | 1.577 | 1.109 | 1.325 | 1.139 | 0 | 0.393 | 0 |
| RRFN | | | | | | | |
| CO1 - WA1-SARS1 | 1.176 | 0.461 | 1.104 | 0.834 | 0 | 0.289 | 0 |
| RRFN | | | | | | | |
| CO2 - WA1-SARS1 | 1.726 | 1.122 | 1.353 | 1.188 | 0 | 0.402 | 0 |
| RRFN | | | | | | | |
| BANAL20247-WA1 | 1.723 | 1.708 | 1.193 | 1.235 | 0 | 0.581 | 0 |
| RRFN | | | | | | | |
| BANAL20247-WA1 | 1.617 | 1.605 | 1.348 | 1.214 | 0 | 0.576 | 0 |
| RRFN | | | | | | | |
| CO2 - WA1-SARS1- | 0.286 | 0.083 | 0.286 | 0.113 | 0 | −0.030 | 0 |
| BANAL20247-WA1 | | | | | | | |
| RR-mosSpFN | | | | | | | |
| co-expression of | 0.850 | 0.165 | 0.635 | 0.100 | 0.000 | 0.222 | −0.148 |
| WA1 SpFN-BQ.1.1 | | | | | | | |
| SpFN-SARS1 SpFN | | | | | | | |
| co-expression of | 0.622 | 0.296 | 0.649 | 0.193 | 0.000 | 0.217 | −0.066 |
| WA1 SpFN-Khosta2 | | | | | | | |
| SpFN-SARS1 SpFN | | | | | | | |
| co-expression of | 0.755 | 0.047 | 0.546 | 0.067 | 0.000 | 0.222 | −0.112 |
| WA1SpFN-Beta | | | | | | | |
| SpFN-BQ.1.1 SpFN | | | | | | | |
| co-expression of | 2.263 | 1.501 | 1.624 | 1.731 | 0.000 | 0.905 | −0.116 |
| WA1-Khosta2- | | | | | | | |
| SARS1 RFN | | | | | | | |
| co-expression of | 1.911 | 1.431 | 1.262 | 1.725 | 0.000 | 0.796 | −0.088 |
| empty FN and Beta | | | | | | | |
| RFN | | | | | | | |
| co-expression of | 1.285 | 0.792 | 0.097 | 1.363 | 0.000 | 0.786 | −0.053 |
| empty FN and BQ1.1 | | | | | | | |
| RFN | | | | | | | |
| co-expression of | −0.044 | 0.651 | 0.181 | 0.941 | 0.000 | 0.705 | 0.021 |
| empty FN and SARS1 | | | | | | | |
| RFN | | | | | | | |
| co-expression of | 0.178 | 0.188 | 0.444 | 0.249 | 1.908 | 0.183 | 0.000 |
| empty FN and MERS | | | | | | | |
| RFN | | | | | | | |
| co-expression of | 1.919 | 1.508 | 0.364 | 1.531 | 0.000 | 0.081 | −0.133 |
| empty FN and | | | | | | | |
| BANAL20247 RFN | | | | | | | |
| co-expression of | 1.632 | 1.935 | 1.719 | 2.143 | 0.000 | 0.901 | 0.000 |
| empty FN, WA1 | | | | | | | |
| RFN, BANAL20247 | | | | | | | |
| RFN and SARS1 | | | | | | | |
| RFN | | | | | | | |
| co-expression of | 1.680 | 1.668 | 1.804 | 0.014 | 0.000 | 0.831 | 0.000 |
| empty FN, WA1 | | | | | | | |
| RFN, Beta RFN, and | | | | | | | |
| BQ1.1 RFN | | | | | | | |

Binding values are response (nm) after 180 seconds measured by biolayer interferometry.

### Example 4: Mosaic Nanoparticle Assembly

Based on the differences observed in the apparent size of RFN or SpFN from different strains using native condition gel electrophoresis (PAGE), the assembly of RBD or Spike trimers from different strains on one ferritin nanoparticle was assessed by comparing the apparent size of monovalent and multivalent RFN (or SpFN). We also evaluated whether co-expression of empty FN with RFNs has a potential benefit on the assembly and secretion efficiency, e.g., decrease the risk of steric hindrance between RBDs on the FN.

### Methods

HeLa cells were plated in 24-well plates at 0.075 million cells/well in 0.5 mL EMEM+10% FBS. Cells were transfected the next day with 1 μg/million cells mRNA constructs with lipofectamine 2000. 24 h post-transfection, the cell lysates and supernatants (to assess secretion) were analyzed in Native-PAGE western blots by probing with anti-ferritin mAb.

### Preparation of Lysates

Cells were harvested the 22-24 hours later and lysed in 125 μL per well of CelLytic M+1×HALT. Lysates were incubated on ice for 10 minutes then cleared in a microcentrifuge at max speed for 10 min at 4° C. 15 μL of lysate were combined with 5 μL of Native Tris-Glycine sample buffer without reducing agent.

### Preparation of Supernatant Samples

Cell culture supernatants were harvested the 22-24 hours after transfection. 15 μL of supernatant were combined with 5 μL of Native Tris-Glycine sample buffer without reducing agent.

### Analysis

The resulting lysate or supernatant samples were run on 4-12% Gradient Native-PAGE at 185V for 75 minutes. Protein was transferred to nitrocellulose membrane. Blots were blocked with Intercept blocking buffer for 1 hour at room temperature. Blots were stained with human anti-ferritin antibody. Antibody staining was done in Intercept Blocking buffer+0.2% Tween 20 for overnight at 4° C. Blots were washed with TBST 3×5 minutes. Blots were then stained with goat anti-human IR800 secondary antibody in Intercept Blocking buffer+0.2% Tween 20 for 1 hr at RT. Blots were washed with TBST 4×5 minutes and scanned on a Licor odyssey.

### Results

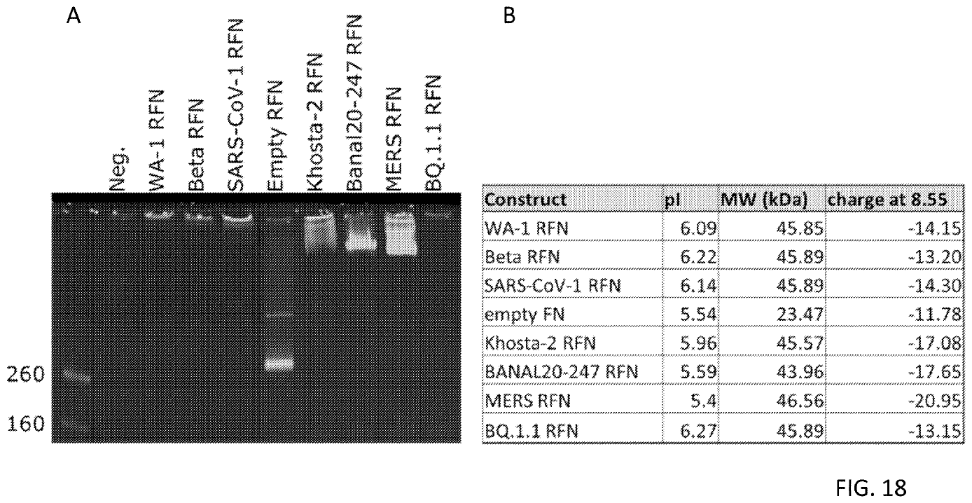

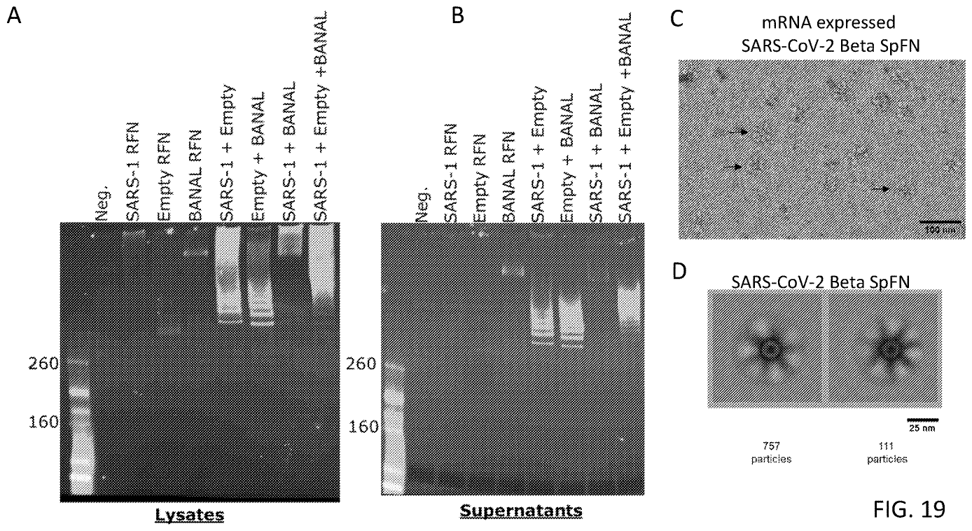

Results of these studies are shown in FIGS. 18 and 19. The data shows that when co-expressing SARS-1 RFN (higher migrating) and BANAL-20-247 RFN (lower migrating), the banding pattern is shifted compared to the pattern observed with the single RFNs to an intermediate pattern. This indicates that there are multiple species most likely composed of different ratios of each construct. This same phenomenon is observed when each RFN is combined with the empty FN (Ferritin alone). This effect is more dramatic as the migration difference is much larger and clearly exhibits multiple species of complexes. Additionally, the lowest migrating multivalent complex is higher than the empty FN indicating that all detectable empty FN is in a complex with at least 1 RFN component. The combination of all three components produces its own distinct banding pattern supporting the formation of complexes containing all three components. The supernatant samples show similar banding patterns indicating that multimember complexes are able to be secreted. Interestingly, the lower migrating complexes appear to be much more efficiently secreted compared to the higher migrating complexes. This indicates that smaller complexes with a higher proportion of empty FN may be more efficiently secreted.

### Example 5—Immunogenicity Study in Naïve Mice

This mouse study focused on mRNA designs spanning RFN and SpFN antigen display paradigms at different antigenic distances (Table 8 below). The vaccine compositions were formulated in Lipid D LNP and evaluated for their immunogenicity in naïve male and female C57BL/6 mice using a 2-dose primary immunization schedule, 4 weeks apart, via the intra-muscular route. Comparison formulations were prepared with monovalent versions of antigens of each component strain of a multivalent mixture, to compare contributions of individual strains and evaluate the impact of multivalent presentation. Additionally, stabilized transmembrane spike proteins (S-2P) for all strains of the monovalent and multivalent formulation were created to assess the contribution of antigen display by the ferritin nanoparticles.

In one set of formulations, the RFN formulations also included a construct encoding the ferritin monomer (without a conjugated antigen), termed an “empty FN.” For one subset of formulations individual mRNA constructs were separately encapsulated in separate LNPs administered in the same composition, and compared to results achieved with a formulations prepared with matched mRNAs co-encapsulated in the same LNP.

### Formulations

For each immunization group (see Table 8) the composition administered was formulated and diluted to a concentration befitting a 1 μg dose per construct per 50 μL. If an mRNA encoding only empty ferritin was included, that mRNA was added at 0.3 μl per construct per 50 μL. Mice receiving control formulations listed as “co-administration” were inoculated with three separate injections, each containing one listed construct encapsulated in an LNP at a dose of 1 μg (total dose 3 μg).

TABLE 8

| Group | Immunogen | Construct_1 | Construct_2 | Construct_3 | Construct_4 | Construct_5 | mRNA dose (μg) |
|---|---|---|---|---|---|---|---|
| 1 | WA-1 RFN + FN | Empty FN | RFN WA-1 | | | | 1.3 |
| 2 | Beta RFN + FN | Empty FN | Beta RFN | | | | 1.3 |
| 3 | BQ.1.1 RFN + FN | Empty FN | BQ.1.1 RFN | — | | | 1.3 |
| 4 | SARS-1 RFN + FN | Empty FN | SARS-1 RFN | | | | 1.3 |
| 5 | Khosta2 RFN + FN | Empty FN | Khosta2 RFN | | | | 1.3 |
| 6 | MERS RFN + FN | Empty FN | MERS RFN | | | | 1.3 |
| 7 | BANAL20247 | Empty FN | BANAL20247 | | | | 1.3 |
| | RFN + FN | | RFN | | | | |
| 8 | Mix D Co-encap | Empty FN | WA-1 RFN | BANAL20247 | SARS-1 | | 3.9 |
| | RFN | | | RFN | RFN | | |
| 9 | Mix C Co-encap | Empty FN | WA-1 RFN | Khosta2 RFN | SARS-1 | | 3.9 |
| | RFN | | | | RFN | | |
| 10 | Mix A Co-encap | Empty FN | WA-1 RFN | Beta RFN | BQ1.1 RFN | | 3.9 |
| | RFN | | | | | | |
| 11 | Mix B Co-encap | Empty FN | WA-1 RFN | BQ1.1 RFN | SARS-1 | | 3.9 |
| | RFN | | | | RFN | | |
| 12 | Mix E Co-encap | Empty FN | WA-1 RFN | SARS-1 RFN | MERS RFN | | 3.9 |
| | RFN | | | | | | |
| 13 | Mix D Co-encap | WA-1 RFN | BANAL20-247 | SARS-1 RFN | | | 3 |
| | RFN (no empty | | RFN | | | | |
| | FN) | | | | | | |
| 14 | Mix D Co-admin | Empty FN | WA-1 RFN | BANAL20-247 | SARS-1 | | 3.9 |
| | RFN + FN coencap | | | RFN | RFN | | |
| 15 | WA-1 S-2P | WA-1 S-2P | | | | | 1 |
| 16 | BQ1.1 S-2P | BQ1.1 S-2P | | | | | 1 |
| 17 | SARS-1 S-2P | SARS-1 S-2P | | | | | 1 |
| 18 | Khosta2 SpFN | Khosta2 SpFN | | | | | 1 |
| 19 | WA-1 SpFN | WA-1 SpFN | | | | | 1 |
| 20 | SARS-1 SpFN | SARS-1 SpFN | | | | | 1 |
| 21 | BQ1.1 SpFN | BQ1.1 SpFN | | | | | 1 |
| 22 | Beta SpFN | Beta SpFN | | | | | 1 |
| 23 | BANAL20247 | BANAL20247 | | | | | 1 |
| | SpFN | SpFN | | | | | |
| 24 | MERS SpFN | MERS SpFN | | | | | 1 |
| 25 | Mix B Co-encap | WA-1 SpFN | BQ1.1 SpFN | SARS-1 SpFN | | | 3 |
| | SpFN | | | | | | |
| 26 | Mix C Co-encap | WA-1 SpFN | Khosta2 SpFN | SARS-1 SpFN | | | 3 |
| | SpFN | | | | | | |
| 27 | Mix A Co-encap | WA-1 SpFN | Beta SpFN | BQ1.1 SpFN | | | 3 |
| | SpFN | | | | | | |
| 28 | Mix D Co-encap | WA-1 SpFN | BANAL20247 | SARS-1 SpFN | | | 3 |
| | SpFN | | SpFN | | | | |
| 29 | Mix E Co-encap | WA-1 SpFN | SARS-1 SpFN | MERS SpFN | | | 3 |
| | SpFN | | | | | | |
| 30 | Mix C Co-admin | WA-1 SpFN | Khosta2 SpFN | SARS-1 SpFN | | | 3 |
| | SpFN | | | | | | |
| 31 | Mix B Co-admin S- | WA-1 S-2P | BQ1.1 S-2P | SARS-1 S-2P | | | 3 |
| | 2P | | | | | | |
| 32 | Empty FN | Empty FN | | | | | 0.3 |
| 33 | Mix D Co-admin | WA-1 RFN | BANAL20247 | SARS-1 RFN | | | 3 |
| | RFN (no empty | | RFN | | | | |
| | FN) | | | | | | |
| 34 | WA-1 RFN (No | WA-1 RFN | | | | | 1 |
| | empty FN) | | | | | | |
| 35 | BANAL20247 | BANAL20247 | | | | | 1 |
| | RFN (No empty | RFN | | | | | |
| | FN) | | | | | | |
| 36 | SARS-1 RFN (No | SARS-1 RFN | | | | | 1 |
| | Empty FN) | | | | | | |
| 37 | Beta S-2P | Beta S-2P | | | | | 1 |
| 38 | BANAL20247 S- | BANAL20247 | | | | | 1 |
| | 2P | S-2P | | | | | |
| 39 | MERS S-2P | MERS S-2P | | | | | 1 |
| 40 | Khosta2 S-2P | Khosta2 S-2P | | | | | 1 |
| 41 | Mix A Co-admin S- | WA-1 S-2P | Beta S-2P | BQ1.1 S-2P | | | 3 |
| | 2P | | | | | | |
| 42 | Mix D Co-admin S- | WA-1 S-2P | BANAL20247 | SARS-1 S-2P | | | 3 |
| | 2P | | S-2P | | | | |
| 43 | Mix E Co-admin S- | WA-1 S-2P | SARS-1 S-2P | MEERS S-2P | | | 3 |
| | 2P | | | | | | |
| 44 | Mix C Co-admin S- | WA-1 S-2P | Khosta2 S-2P | SARS-1 S-2P | | | 3 |
| | 2P | | | | | | |
| 45 | Mix C Patterned | Empty FN | Khosta2-WA1 | WA1-Khosta2 | SARS1- | WA1- | 4.3 |
| | RRFN + FN | | RRFN | RRFN | WA1 RRFN | SARS1 | |
| | | | | | | RRFN | |
| 46 | Mix D RR-SpFN | WA1- | | | | | 1 |
| | | BANAL20247- | | | | | |
| | | SARS1-WA1 | | | | | |
| | | RR-mosSpFN | | | | | |
| 47 | Mix D RR-SpFN | WA1- | | | | | 3 |
| | | BANAL20247- | | | | | |
| | | SARS1-WA1 | | | | | |
| | | RR-mosSpFN | | | | | |
| 48 | Mix D + Mix F | WA1- | SARS1- | | | | 2 |
| | Patterned RR-SpFN | BANAL20247- | BANAL20247- | | | | |
| | | SARS1-WA1 | Khosta2-WA1 | | | | |
| | | RR-mosSpFN | RR-mosSpFN | | | | |
| 49 | Mix D + Mix F | WA1- | Khosta2- | | | | 2 |
| | Patterned RR-SpFN | BANAL20247- | BANAL20247- | | | | |
| | | SARS1-WA1 | SARS1-WA1 | | | | |
| | | RR-mosSpFN | RR-mosSpFN | | | | |
| 50 | Mix D + Mix F | WA1- | Khosta2- | SARS1- | | | 3 |
| | Patterned RR-SpFN | BANAL20247- | BANAL20247- | BANAL20247- | | | |
| | | SARS1-WA1 | SARS1-WA1 | Khosta2-WA1 | | | |
| | | RR-mosSpFN | RR-mosSpFN | RR-mosSpFN | | | |
| 51 | Khosta-2 WA-1 R- | Khosta-2 WA- | | | | | 1 |
| | SpFN | 1 R-SpFN | | | | | |

SARS-1 = SARS-CoV-1
All mRNA constructs were encapsulated in Lipid D LNPs.
Groups 8-12: Co-encapsulated mRNA encoding ferritin conjugated to RBD and empty ferritin (RFN + FN)
Groups 25-31: Co-encapsulated mRNA encoding ferritin conjugated to spike proteins (SpFN)
Group 45: Co-encapsulated mRNA encoding ferritin conjugated to two sequential RBDs separated by linkers (RRFN)
Groups 46-47: Monovalent mRNA encoding ferritin conjugated to Spike conjugated to two or more sequential RBDs separated by linkers at two doses, 1 μg and 3 μg. (RR-MosSpFN)
Groups 48-50: Co-encapsulated multiple mRNAs encoding ferritin conjugated to Spike conjugated to two or more sequential RBDs separated by linkers including strains from Mix D on one construct and Mix F on one (Groups 48, 49 - note different order of RBDs) or two (Group 50) constructs (RR-MosSpFN)
Group 51: Co-encapsulated mRNA encoding ferritin conjugated to Spike conjugated to RBD (monovalent with WA-1 spike and Khosta-2 RBD similar to Mix D lacking SARS-CoV-1 immunogen)
For control groups, mRNA encoding the following constructs were prepared:
Groups 1-7, 18-24: Monovalent ferritin conjugated to RBD (RFN) or spike protein (SpFN)
Groups 13, 33-36: Monovalent or multivalent (13, 33) ferritin conjugated to RBD without empty ferritin (RFN)
Groups 15-17, 37-40: Monovalent transmembrane stabilized Spike proteins
Groups 31, 41-44: Multivalent transmembrane stabilized spike proteins encapsulated in separate LNPs for co-administration
Groups 14, 30, 33: Ferritin conjugated to RBD (RFN) (Group 33), ferritin conjugated to RBD co-encapsulated with empty ferritin (RFN + FN) (Group 14), or Spike protein (SpFN) encapsulated in separate LNPs for co-administrations (Group 30)
Group 32: empty ferritin

The sera collected two weeks after the second immunization were tested for neutralization capacity against a panel of strains selected for their clinical relevance. This panel included SARS-CoV-2 (Clade 1b) variants (WA-1, Delta, Beta, BA.5, BQ.1.1, XBB.1.5) and a representative strain from SARS-CoV-1. A Merbicovirus readout was also included.

### Pseudovirus Neutralization Assay

The Spike protein (S) expression plasmid sequences for SARS-CoV-2 and SARS-CoV-1 were codon optimized and modified to remove an 18 amino acid endoplasmic reticulum retention signal in the cytoplasmic tail in the case of SARS-CoV-2, and a 28 amino acid deletion in the cytoplasmic tail in the case of SARS-CoV. This allowed increased S incorporation into pseudovirions (PSV) and thereby improve infectivity. Virions pseudotyped with the vesicular stomatitis virus (VSV) Gprotein were used as a non-specific control. SARS-CoV-2 pseudovirions (PSV) were produced by co-transfection of HEK293T/17 cells with a SARS-CoV-2 S plasmid (pcDNA3.4) and an HIV-1 NL4-3 luciferase reporter plasmid (The reagent was obtained through the NIH HIV Reagent Program, Division of AIDS, NIAID, NIH: Human Immunodeficiency Virus 1 (HIV-1) NL4-3 ΔEnv Vpr Luciferase Reporter Vector (pNL4-3.Luc.R-E-), ARP-3418, contributed by Drs. Nathaniel Landau and Aaron Diamond).

Infectivity and neutralization titers were determined using ACE2-expressing HEK293 target cells (Integral Molecular) in a semi-automated assay format using robotic liquid handling (Biomek NXp Beckman Coulter). Test sera were diluted 1:40 in growth medium and serially diluted, then 25 μL/well was added to a white 96-well plate. An equal volume of diluted SARS-CoV-2 PSV was added to each well and plates were incubated for 1 hour at 37° C. Target cells were added to each well (40,000 cells/well) and plates were incubated for an additional 48 hours. RLUs were measured with the EnVision Multimode Plate Reader (Perkin Elmer, Waltham, MA) using the Bright-Glo Luciferase Assay System (Promega Corporation, Madison, WI). Neutralization dose-response curves were fitted by nonlinear regression with a five-parameter curve fit using the LabKey Server®, and the final titers reported as the reciprocal of the dilution of serum necessary to achieve 50% neutralization (ID50, 50% inhibitory dilution) and 80% neutralization (ID80, 80% inhibitory dilution).

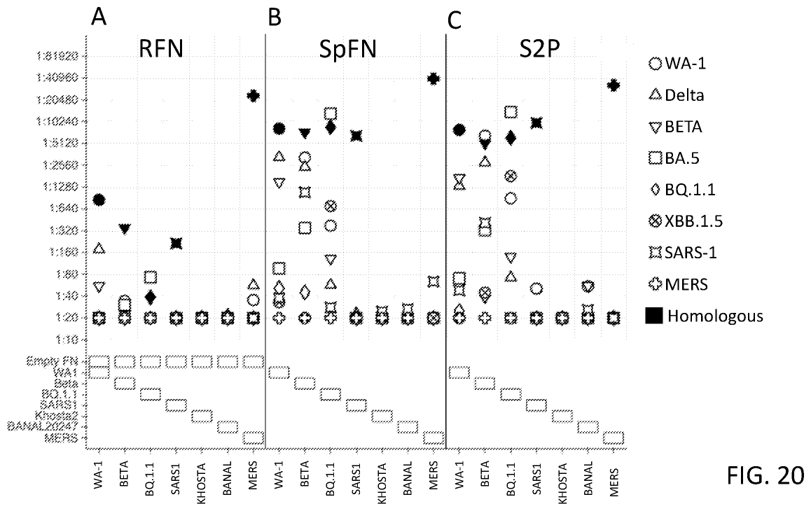

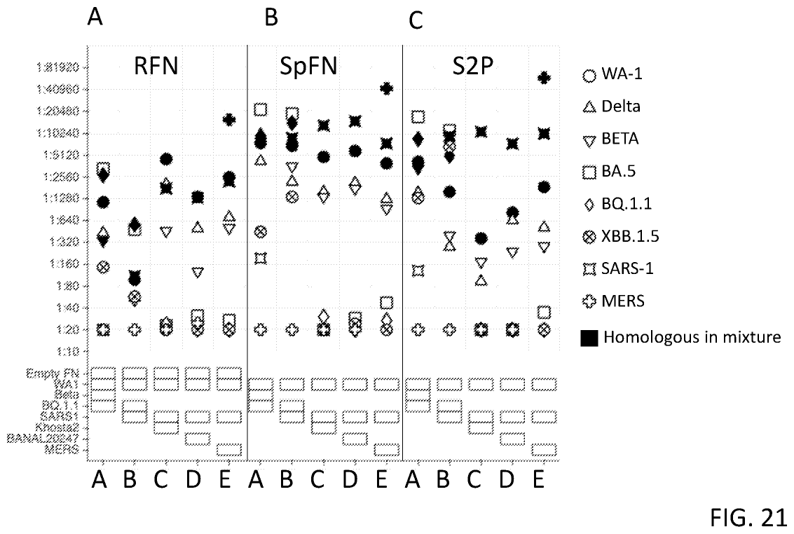

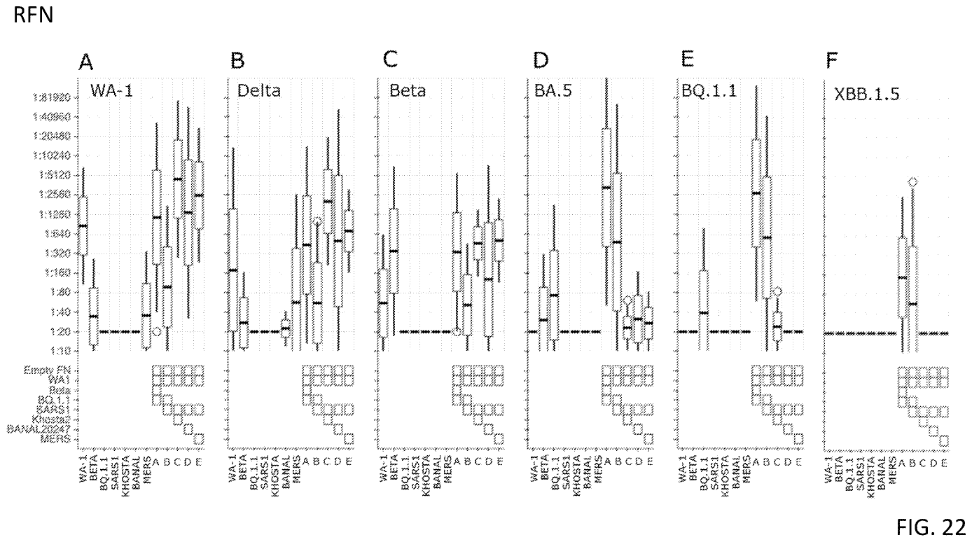

The results for monovalent constructs and multivalent mixes A through E formulations in either the RFN, SpFN, or S2P antigen presentation format are summarized in FIGS. 20 and 21. Statistical representation of the mouse group titers for mice inoculated with monovalent or multivalent mixes A through E in either RFN, SpFN, or S2P antigen presentation format are provided in FIGS. 22, 23, and 24. Generally, across antigen presentation formats, monovalent strains provided substantial neutralizing titers to similar strains (FIG. 20). WA-1 (Groups 1, 15, 19) neutralizes WA-1, Delta, and Beta. For monovalent controls, the greatest breadth across Clades 1a and 1b was seen with Beta immunization (Groups 2, 22, 37) from which sera was observed to neutralize WA-1, Delta, and Beta. In addition, some mice had substantial titer to two sequentially distant Omicron strains, BA.5 and BQ.1.1, and Clade 1a SARS-CoV-1 pseudoviruses. Sera from BQ.1.1 immunized mice (Groups 3, 16, 21) elicited high titers to tested Omicron strains, BA.5 and BQ.1.1 with diminished titers to Beta, Delta, and WA-1. These three monovalent controls establish that the selected Clade 1b strains span the antigenic landscape of the SARS-CoV-2 pandemic from original strain to recent Omicron strains. For other antigens including SARS-CoV-1, Khosta-2, BANAL20-247, and MERS, immune mouse sera did not elicit any neutralizing titers to Clade 1b pseudoviruses tested. SARS-CoV-1 (Groups 4, 17, 20) and MERS (6, 24, 39) strains elicited high titers only to their homologous pseudovirus (FIG. 20).

Mix A (WA-1, Beta, BQ.1.1) (Groups 10, 27, 41) multivalent formulations including three-component monovalent strains, generated breadth across clade 1b demonstrated by high titer observations against all clade 1b sarbecoviruses tested, especially for the SpFN and S-2P antigen display methods (Groups 27 and 41 respectively). Mix B (WA-1, BQ.1.1, SARS-CoV-1) (Groups 11, 25, 31) generated high titers across all clade 1b sarbecoviruses as well but also provided at least homologous coverage within clade 1a. Mixes including WA-1, SARS-CoV-1 and one of Khosta-2 (Mix C; Groups 9, 26, 44), BANAL20-247 (Mix D; Groups 8, 28, 42), or MERS (Mix E; Groups 12, 29, 43) demonstrated titers similar to WA-1 for clade 1b and SARS-CoV-1 for clade 1a with little interference from the additional strain (FIGS. 21-24).

Enhanced Clade 1b Neutralization Titers with Mix B Immunization

Sera generated using the antigen designs and immunization strategies disclosed here may be considered to have an enhanced breadth of immune response if they have increased neutralization titers (as measured by pseudoneutralization assay) to one or more sarbecovirus strains not included as a vaccine component. The enhanced effect of multivalent antigen presentation as described herein can be assessed by comparing the pseudoneutralization titers between mouse groups inoculated with multivalent formulations and their concordant monovalent formulations in the same antigenic presentation.

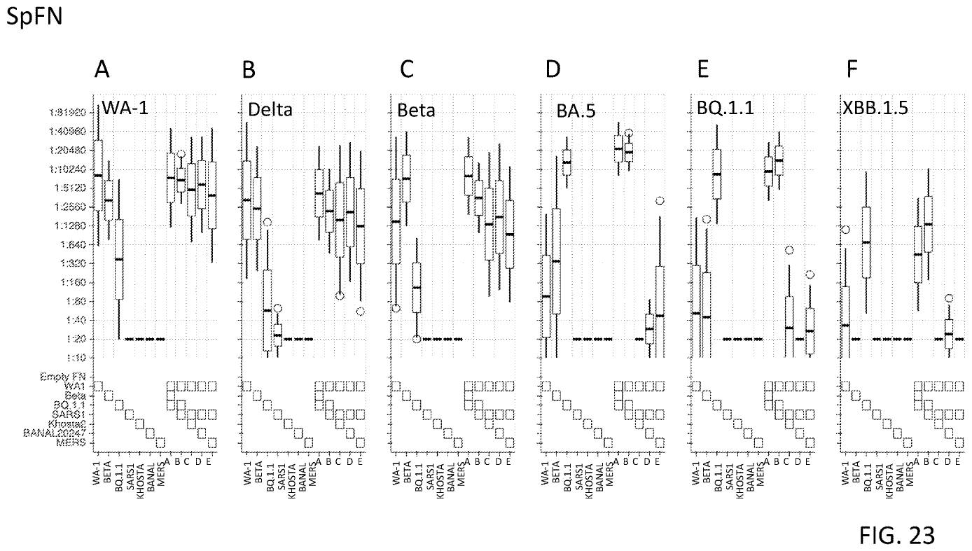

Neutralization titers against the Beta strain elicited by the medium antigenic distance set containing WA-1, BQ.1.1, and SARS-CoV-1 (Mix B) in the co-encapsulated SpFN antigenic presentation format (Groups 25), was enhanced compared to those elicited by WA-1, BQ.1.1, or SARS-1 monovalent SpFN (Groups 19-21) (FIG. 23, Panel C). The titer to the Beta pseudovirus for Mix B SpFN was also improved (per GMT titer and minimum titer per mouse group) compared to any other multi- or monovalent not containing the Beta strain across all antigen presentations.

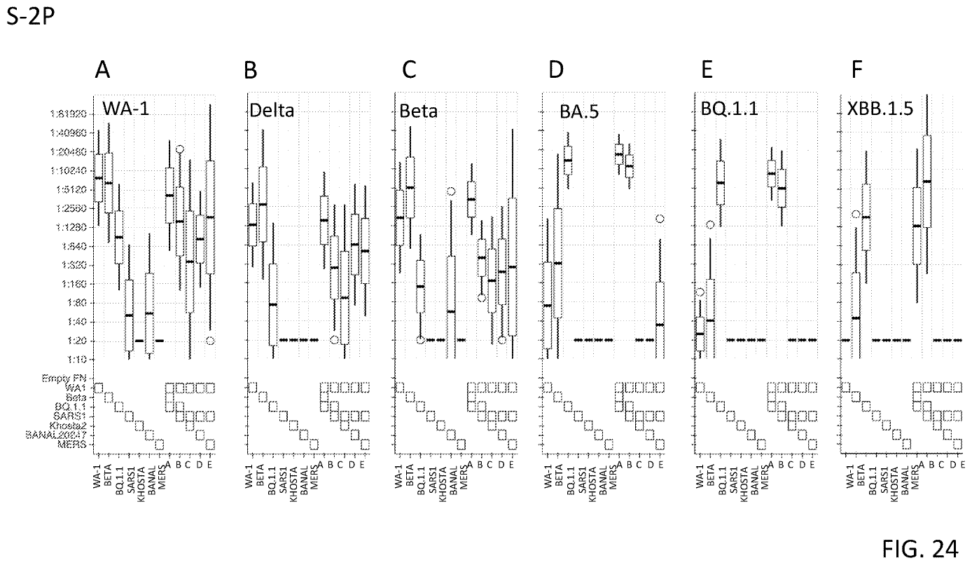

Without being bound by theory, this enhanced titer to the Beta pseudovirus may be mediated by generation of antibodies that bind to a broader range of common epitopes (e.g. antibodies tolerant to accumulated mutations between strains) to allow for simultaneous binding to two of the WA-1, BQ.1.1, and SARS-CoV-1 SpFN constructs displayed on the same nanoparticle. This result is evidence of enhanced immune response breadth because the component strains in Mix B do not contain a Beta strain antigen, but still elicited a neutralization titer of more than 1:2560. Similarly, immunizations with either co-encapsulated SpFN or co-administered S-2P Mix B also trend towards enhanced cross-neutralization of XBB.1.5 strain (FIGS. 23F and 24F). Immunization with co-encapsulated SpFN Mix B also trends towards enhanced homologous titer for BQ.1.1 (FIG. 23, Panel E).

Although others have reported increased titers to several SARS-CoV-1 strains through inoculation with a multivalent display system presenting eight SARS-CoV-1 and SARS CoV-2 strains, all observed titers in those reports were consistent with vaccination by the component antigens. In other words, any breadth increase was simply mediated by each individual antigen present rather than cross-binding between antigens. In contrast, the results here show increased breadth that is necessarily mediated by peripheral binding between one or more multivalent antigens.

Multivalent SpFN Antigen Presentation Format Elicits Higher Neutralization Titers than Multivalent S-2P Format

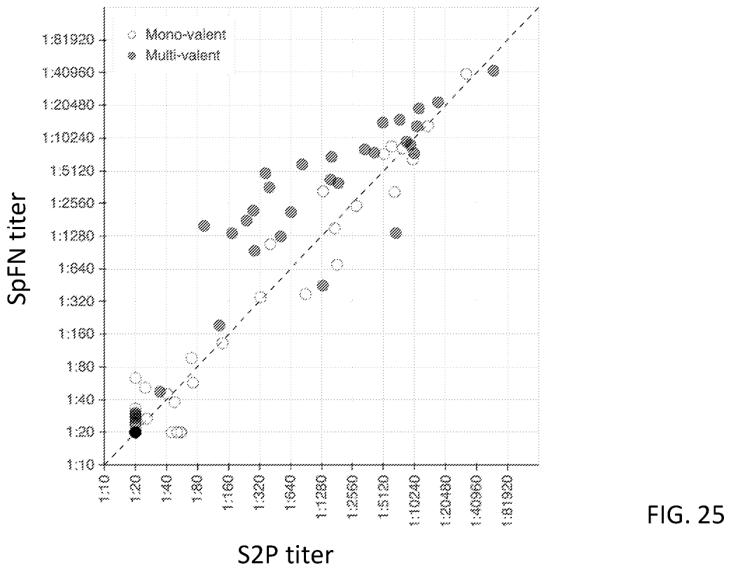

Comparing the pseudoneutralization titers between mouse groups inoculated with the Mix B antigenic distance set presented as stabilized transmembrane proteins (S-2P) and SpFN, demonstrates the benefit of the ferritin antigen presentation system. FIG. 25 compares pseudoneutralization titers between mono- and multivalent SpFN groups and S-2P groups. For monovalent groups (open circles; S-2P, monovalent groups 15-17, 37-40; SpFN, monovalent groups 18-24) neutralization titers trending along the x=y line (dashed line) were observed, suggesting that titers are approximately equal across antigen presentations. However, the multivalent groups (filled circles; S-2P, groups 31, 41-44; SpFN, groups 25-29) showed improved pseudoneutralization titers for the SpFN antigen presentation, revealed by titers above the x=y line (dashed line) in four out of the six strains tested (with the exception of SARS-CoV-1 and XBB.1.5, not indicated in FIG. 25).

### Co-Encapsulation and Co-Administered Multivalent Antigens

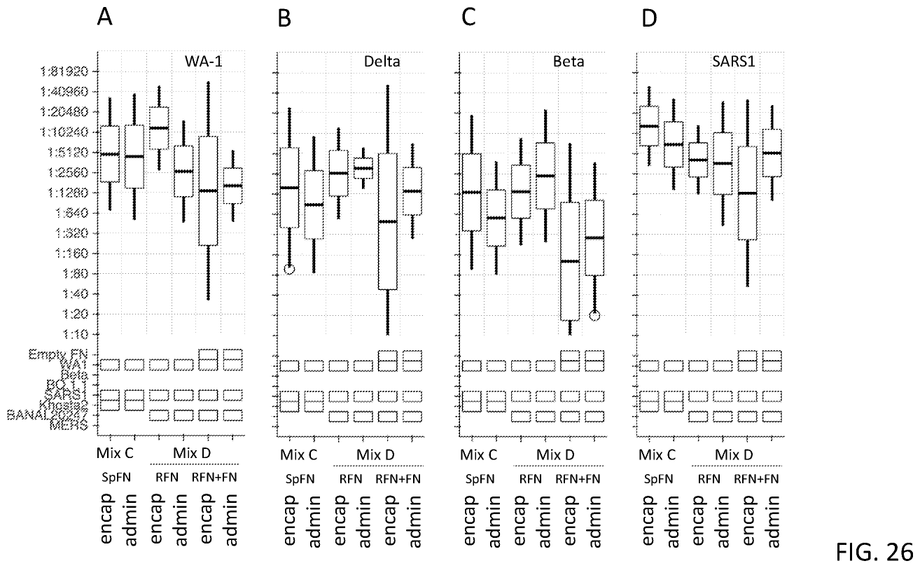

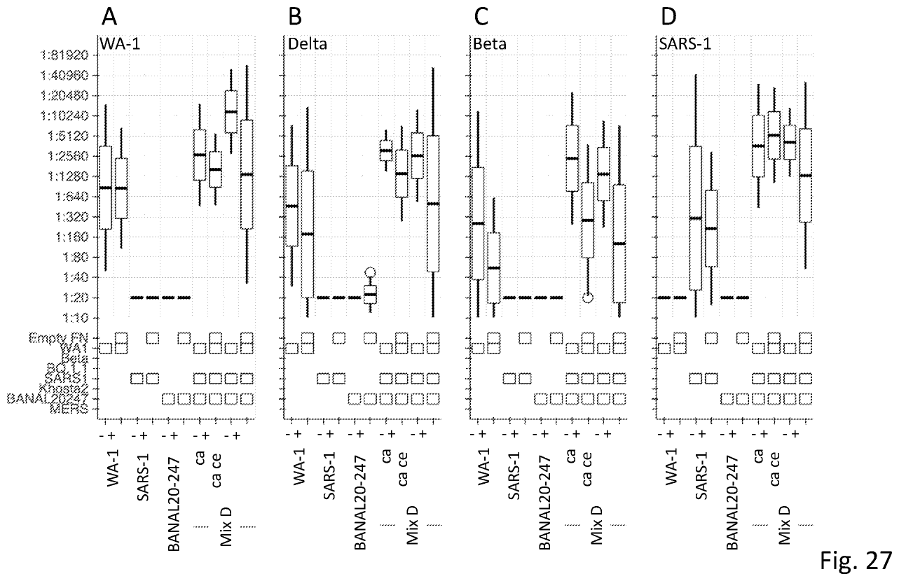

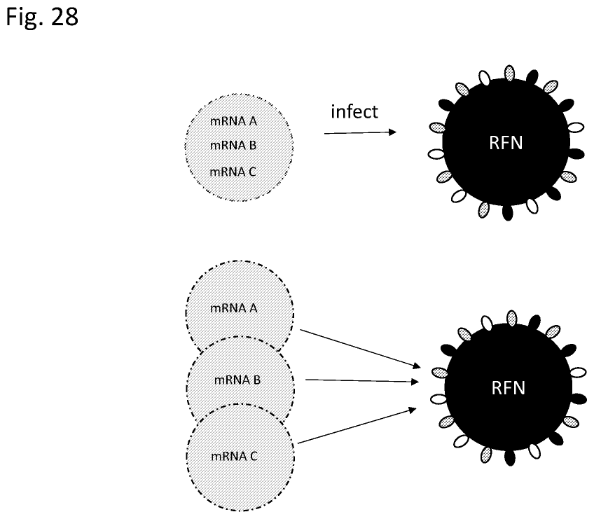

Comparison of results obtained in mouse groups inoculated with LNP formulations of co-encapsulated mRNA vs. LNP formulations of mixtures of separately encapsulated mRNA (e.g., with each mRNA molecule separately encapsulated in LNPs and co-administered), did not reveal a significant difference between co-encapsulation and separate encapsulation. Pseudoneutralization titers were very similar between all three co-administered mixtures and their co-encapsulated counterparts. This suggests that individual mRNA/LNP formulations of each type are taken up by each cell, resulting in similar expression patterns and nanoparticle formation (FIGS. 26A-26D).

### Neutralization Titers Elicited by RFN, SpFN and S2P Antigen Presentation Formats

Comparing matched strain sets between these RFN, SpFN, and S-2P antigen display formats, SpFN formulations showed improved titers across all Clade 1a and 1b readouts (FIGS. 20-23). As an exception, the MERS readout showed strong immunogenicity for both RFN and SpFN constructs and formulations containing a MERS construct.

### Example 6: Multivalent Antigens as Booster Vaccine

The ability of multivalent compositions to elicit cross-neutralizing antibodies upon booster vaccination will be evaluated in BALB/C female mice (n=8) primed with mRNA encoding WA-1 at day 0 and day 21, and boosted (1^{st} boost) 3 months later with the formulations and dose levels as shown in Table 9. The mice will be bled one day before the boost to assess pre-boost titers in all groups. Animals will then be bled two weeks after the boost to determine and compare across the groups post-boost titers. Commercial S2-P XBB.1.5 SARS CoV-2 (SoC) vaccine will be included in this study as a comparator for the proposed novel booster formulations.

TABLE 9

| | Prime | | | | | | | 2^{nd} Boost | |
|---|---|---|---|---|---|---|---|---|---|
| | Antigen | Dose | Antigen | 1^{st} Boost | | | Dose | | Dose |
| Group | D 0/D 21 | (μg) | Type | Construct_1 | Construct_2 | Construct_3 | (μg) | Construct | (μg) |
| 1 | Prime: | 1 | Multivalent | WA-1 SpFN | BQ.1.1 | SARS-1 | 3 | | |
| | WA-1 | | SpFN | | SpFN | SpFN | | | |
| 2 | S-2P | | | Beta SpFN | BQ.1.1 | SARS1 | 3 | | |
| | mRNA | | | | SpFN | SpFN | | | |
| 3 | | | | BQ.1.1 | SARS-1 | Khosta2 | 3 | | |
| | | | | SpFN | SpFN | SpFN | | | |
| 4 | | | Monovalent | BQ.1.1 | | | 1 | | |
| | | | SpFN | SpFN | | | | | |
| 5 | | | | WA-1 SpFN | | | 1 | WA-1 SpFN | 1 |
| 6 | | | Multivalent | WA-1 S-2P | BQ.1.1 S-2P | SARS-1 S- | 3 | | |
| | | | S-2P | | | 2P | | | |
| 7 | | | | Beta S-2P | BQ1.1 S-2P | SARS1 S- | 3 | | |
| | | | | | | 2P | | | |
| 8 | | | | BQ.1.1 S-2P | SARS-1 S- | Khosta2 S- | 3 | | |
| | | | | | 2P | 2P | | | |
| 9 | | | | Beta S-2P | XBB.1.5 S- | SARS1 S- | 3 | | |
| | | | | | 2P | 2P | | | |
| 10 | | | Monovalent | XBB.1.5 S- | | | 1 | | |
| | | | S2P | 2P | | | | | |
| 11 | | | | WA-1 S-2P | | | 1 | WA-1 S-2P | 1 |
| 12 | | | Multivalent | Beta SpFN | BQ.1.1 | SARS1 | 3 | Beta/BQ.1.1/ | 3 |
| | | | SpFN | | SpFN | SpFN | | SARS1 | |
| | | | | | | | | SpFN | |
| 13 | | | Multivalent | Beta S-2P | BQ.1.1 S-2P | SARS1 S- | 3 | Beta/BQ.1.1/ | 3 |
| | | | S-2P | | | 2P | | SARS1 S-2P | |
| 14 | | | | Commercial | | | | | |
| | | | | S2P vaccine | | | | | |
| 15 | | 0.25 (low | Multivalent | WA-1 SpFN | BQ.1.1 | SARS-1 | | | |
| | | dose | | | SpFN | SpFN | | | |
| 16 | | groups) | | Beta SpFN | BQ1.1 | SARS1 | | | |
| | | | | | SpFN | SpFN | | | |
| 17 | | | | WA-1 S-2P | BQ.1.1 S-2P | SARS-1 S- | | | |
| | | | | | | 2P | | | |
| 18 | | | | Beta S-2P | BQ1.1 S-2P | SARS1 S- | | | |
| | | | | | | 2P | | | |

| | |
|---|---|
| 19 | PBS |

The effects of a second boost on potential further increase in the breadth of neutralizing antibodies will be tested for the selected S-2P and SpFN multivalent formulation (Beta, BQ.1.1, SARS-CoV-1) and monovalent controls WA-1 S-2P and SpFN. The second boost will be introduced 1 month after the first boost. Mice will be bled one day before the second boost is administered and then bled again two weeks after the second boost to assess pre- and post-second boost titers. All bleeds from this study will be tested for their preudoneutralization titers against a defined panel of strains across Clades 1a, 1b, 3 and 4 selected for their clinical relevance.

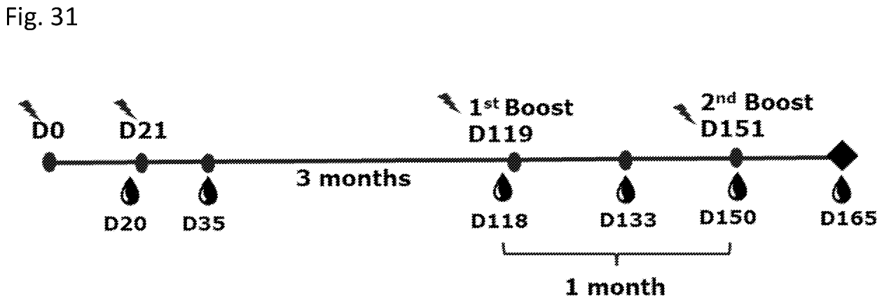

A specific dosing regimen will be as follows: Priming: Day 0 and D21; Bleed: Day 35; Pre-boost bleed: about one day before 1st boost; 1st Boost: 3 months after Day 21 (D119); Bleed: 2 weeks after 1st boost (D133); Pre-2nd boost bleed: about one day before 2nd boost (D150); 2nd boost: one month after 1st boost (D151). There will be 8 mice in each group. The dosing regimen is illustrated in FIG. 31.

For this study, multivalent compositions will comprise admixtures of LNPs separately encapsulating mRNA molecules encoding a given antigen, but another study may employ multivalent compositions that comprise multiple mRNA molecules co-encapsulated in a single LNP.

TABLE 6

| | | | | SEQ |
|---|---|---|---|---|
| Construct | | | | ID |
| Name | Design format | Construct description | Protein sequence | NO |
| M3.1-RBD- | RBD-Ferritin | His8-3c-M3-RBD-Y499R- | HHHHHHHHGPLEVLFQGPEGVECDFSPLLSGTPPQVYNFKRLVFTNCNYNLTKLLSLFSVNDFTCSQI | 29 |
| Ferr | | Ferritin | SPAAIASNCYSSLILDYFSYPLSMKSDLSVSSAGPISQFNYKQSFSNPTCLILATVPHNLTTITKPLKYSRI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.2-RBD- | RBD-Ferritin | His8-3c-M3-RBD-L506R- | HHHHHHHHGPLEVLFQGPEGVECDFSPLLSGTPPQVYNFKRLVFTNCNYNLTKLLSLFSVNDFTCSQI | 30 |
| Ferr | | Ferritin | SPAAIASNCYSSLILDYFSYPLSMKSDLSVSSAGPISQFNYKQSFSNPTCLILATVPHNLTTITKPLKYSYI | |
| | | | NKCSRRLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.3-RBD- | RBD-Ferritin | His8-3c-M3-RBD-V555R- | HHHHHHHHGPLEVLFQGPEGVECDFSPLLSGTPPQVYNFKRLVFTNCNYNLTKLLSLFSVNDFTCSQI | 31 |
| Ferr | | Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLRASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.4-RBD- | RBD-Ferritin | His8-3c-M3-RBD-W553R- | HHHHHHHHGPLEVLFQGPEGVECDFSPLLSGTPPQVYNFKRLVFTNCNYNLTKLLSLFSVNDFTCSQI | 32 |
| Ferr | | Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGRLVASGSTVAMTEQL | |
| | | | QMGFGITVQNGTDTNSVCPKGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| M3.5-RBD- | RBD-Ferritin | His8-3c-M3-RBD-Y523K- | HHHHHHHHGPLEVLFQGPEGVECDFSPLLSGTPPQVYNFKRLVFTNCNYNLTKLLSLFSVNDFTCSQI | 33 |
| Ferr | | Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQKSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.6-RBD- | RBD-Ferritin | His8-3c-M3-RBD-Y523R- | HHHHHHHHGPLEVLFQGPEGVECDFSPLLSGTPPQVYNFKRLVFTNCNYNLTKLLSLFSVNDFTCSQI | 34 |
| Ferr | | Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQRSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.7-RBD- | RBD-Ferritin | His8-3c-M3-RBD-W535A- | HHHHHHHHGPLEVLFQGPEGVECDFSPLLSGTPPQVYNFKRLVFTNCNYNLTKLLSLFSVNDFTCSQI | 35 |
| Ferr | | Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVAEDGDYYRKQLSPLEGGGWLVASGSTVAMTEQL | |
| | | | QMGFGITVQNGTDTNSVCPKGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| M3.8-RBD- | RBD-Ferritin | His8-3c-M3-RBD- | HHHHHHHHGPLEVLFQGPEGVECDFSPLLSGTPPQVYNFKRLVFTNCNYNLTKLLSNFSVNDFTCSQI | 36 |
| Ferr | | L417N(glycan)-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.9-RBD- | RBD-Ferritin | His8-3c-M3-RBD-A434R- | HHHHHHHHGPLEVLFQGPEGVECDFSPLLSGTPPQVYNFKRLVFTNCNYNLTKLLSLFSVNDFTCSQI | 37 |
| Ferr | | Ferritin | SPAAIRSNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.10-RBD- | RBD-Ferritin | His8-3c-M3-RBD-A434N- | HHHHHHHHGPLEVLFQGPEGVECDFSPLLSGTPPQVYNFKRLVFTNCNYNLTKLLSLFSVNDFTCSQI | 38 |
| Ferr | | N436T (glycan)-Ferritin | SPAAINSTCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.11-RBD- | RBD-Ferritin | M3.2 + 3.6-RBD-Ferr | HHHHHHHHGPLEVLFQGPEGVECDFSPLLSGTPPQVYNFKRLVFTNCNYNLTKLLSLFSVNDFTCSQI | 39 |
| Ferr | | | SPAAIASNCYSSLILDYFSYPLSMKSDLSVSSAGPISQFNYKQSFSNPTCLILATVPHNLTTITKPLKYSYI | |
| | | | NKCSRRLSDDRTEVPQLVNANQRSPCVSIVPSTVWEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| M3.12-RBD- | RBD-Ferritin | M3.6 + 3.7-RBD-Ferritin | HHHHHHHHGPLEVLFQGPEGVECDFSPLLSGTPPQVYNFKRLVFTNCNYNLTKLLSLFSVNDFTCSQI | 40 |
| Ferr | | combo | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQRSPCVSIVPSTVAEDGDYYRKQLSPLEGGGWLVASGSTVAMTEQL | |
| | | | QMGFGITVQNGTDTNSVCPKGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| M3.13-RBD- | RBD-Ferritin | M3.6+3.7+3.2-RBD-Ferritin | HHHHHHHHGPLEVLFQGPEGVECDFSPLLSGTPPQVYNFKRLVFTNCNYNLTKLLSLFSVNDFTCSQI | 41 |
| Ferr | | combo | SPAAIASNCYSSLILDYFSYPLSMKSDLSVSSAGPISQFNYKQSFSNPTCLILATVPHNLTTITKPLKYSYI | |
| | | | NKCSRRLSDDRTEVPQLVNANQRSPCVSIVPSTVAEDGDYYRKQLSPLEGGGWLVASGSTVAMTEQ | |
| | | | LQMGFGITVQNGTDTNSVCPKGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| pCoV254 | RBD-Ferritin | RFN with omicron RBD but | HHHHHHHHGPLEVLFQGPNITNLCPFDEVFNATRFASVYAWNRKRISNCVADYSVLYNLAPFFTFKCY | 42 |
| | | including the solubilizing | GVSPTKLNDLCFTNVYADSFVIRGDEVRQIAPGQTGNIADYNYKLPDDFTGCVIAWNSNKLDSKVSGN | |
| | | mutations from pCoV127 | YNYLYRLFRKSNLKPFERDISTEIYQAGNKPCNGVAGFNCYAPLRSYSFRPTYGVGHQPYRVVVLSFE | |
| | | | NKSAPATVCGPGSGGGGESQVRQQFSKDIEKLLNEQVNKEMQSSNLYMSMSSWCYTHSLDGAGLF | |
| | | | LFDHAAEEYEHAKKLIIFLNENNVPVQLTSISAPEHKFEGLTQIFQKAYEHEQHISESINNIVDHAIKSKDH | |
| | | | ATFNFLQWYVAEQHEEEVLFKDILDKIELIGNENHGLYLADQYVKGIAKSRKSGS | |
| pCoV256 | RBD-NTD-Ferritin | His8-3c-RBD-GSGGSG- | HHHHHHHHGPLEVLFQGPNITNLCPFGEVFNATRFASVYAWNRKRISNCVADYSVLYNSASFSTFKCY | 43 |
| | | NTD(delta)-Ferrritin | GVSPTKLNDLCFTNVYADSFVIRGDEVRQIAPGQTGKIADYNYKLPDDFTGCVIAWNSNNLDSKVGGN | |
| | | | YNYLRRLFRKSNLKPFERDISTEIYQAGSTPCNGVEGFNCYFPLQSYGFQPTNGVGYQPYRVVVLSFE | |
| | | | NKSAPATVCGPGSGGSGSSQCVNLRTRTQLPPAYTNSFTRGVYYPDKVFRSSVLHSTQDLFLPFFSN | |
| | | | VTWFHAIHVSGTNGTKRFDNPVLPFNDGVYFASIEKSNIIRGWIFGTTLDSKTQSLLIVNNATNVVIKVC | |
| | | | EFQFCNDPFLDVYYHKNNKSWMESGVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLGSGGGGESQVRQQFSKDIEKLLNEQVNKEMQSSN | |
| | | | LYMSMSSWCYTHSLDGAGLFLFDHAAEEYEHAKKLIIFLNENNVPVQLTSISAPEHKFEGLTQIFQKAY | |
| | | | EHEQHISESINNIVDHAIKSKDHATFNFLQWYVAEQHEEEVLFKDILDKIELIGNENHGLYLADQYVKGIA | |
| | | | KSRKSGS | |
| pCoV263 | RBD-RBD-Ferritin | His8-3c-SARS1RBD-GSGGSG- | HHHHHHHHGPLEVLFQGPNITNLCPFGEVFNATKFPSVYAWERKKISNCVADYSVLYNSTFFSTFKCY | 44 |
| | | RBD(131)-Ferritin | GVSATKLNDLCFSNVYADSFVVKGDDVRQIAPGQTGVIADYNYKLPDDFMGCVLAWNTRNIDATSTG | |
| | | | NYNYKYRYLRHGKLRPFERDISNVPFSPDGKPCTPPALNCYWPLNDYGFYTTTGIGYQPYRVVVLSFE | |
| | | | LLNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoV264 | RBD-RBD-Ferritin | His8-3c-SARS1RBD-GSGGSG- | HHHHHHHHGPLEVLFQGPNITNLCPFGEVFNATKFPSVYAWERKKISNCVADYSVLYNSTFFSTFKCY | 45 |
| | | RBD(127)-Ferritin | GVSATKLNDLCFSNVYADSFVVKGDDVRQIAPGQTGVIADYNYKLPDDFMGCVLAWNTRNIDATSTG | |
| | | | NYNYKYRYLRHGKLRPFERDISNVPFSPDGKPCTPPALNCYWPLNDYGFYTTTGIGYQPYRVVVLSFE | |
| | | | LLNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLYRLFRKSNLKPFERDISTEIYQAGSTPCNGVEGFNCYAPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoV267 | RBD-RBD-Ferritin | His8-3c-RBD(131)-GSGGSG- | HHHHHHHHGPLEVLFQGPNITNLCPFGEVFNATRFASVYAWNRKRISNCVADYSVLYNSASFSTFKCY | 46 |
| | | M3.7RBD-Ferritin | GVSPTKLNDLCFTNVYADSFVIRGDEVRQIAPGQTGKIADYNYKLPDDFTGCVIAWNSNNLDSKVGGN | |
| | | | YNYLRRLFRKSNLKPFERDISTEIYQAGSTPCNGVEGFNCYFPLQSYGFQPTNGVGYQPYRVVVLSFE | |
| | | | NKSAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVAEDGDYYRKQLSPLEGGGWLVASGSTVAMTEQLQ | |
| | | | MGFGITVQNGTDTNSVCPKGSGGGGESQVRQQFSKDIEKLLNEQVNKEMQSSNLYMSMSSWCYTH | |
| | | | SLDGAGLFLFDHAAEEYEHAKKLIIFLNENNVPVQLTSISAPEHKFEGLTQIFQKAYEHEQHISESINNIV | |
| | | | DHAIKSKDHATFNFLQWYVAEQHEEEVLFKDILDKIELIGNENHGLYLADQYVKGIAKSRKSGS | |
| pCoV268 | RBD-RBD-Ferritin | His8-3c-RBD(127)-GSGGSG- | HHHHHHHHGPLEVLFQGPNITNLCPFGEVFNATRFASVYAWNRKRISNCVADYSVLYNSASFSTFKCY | 47 |
| | | M3.7RBD-Ferritin | GVSPTKLNDLCFTNVYADSFVIRGDEVRQIAPGQTGKIADYNYKLPDDFTGCVIAWNSNNLDSKVGGN | |
| | | | YNYLYRLFRKSNLKPFERDISTEIYQAGSTPCNGVEGFNCYAPLQSYGFQPTNGVGYQPYRVVVLSFE | |
| | | | NKSAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVAEDGDYYRKQLSPLEGGGWLVASGSTVAMTEQLQ | |
| | | | MGFGITVQNGTDTNSVCPKGSGGGGESQVRQQFSKDIEKLLNEQVNKEMQSSNLYMSMSSWCYTH | |
| | | | SLDGAGLFLFDHAAEEYEHAKKLIIFLNENNVPVQLTSISAPEHKFEGLTQIFQKAYEHEQHISESINNIV | |
| | | | DHAIKSKDHATFNFLQWYVAEQHEEEVLFKDILDKIELIGNENHGLYLADQYVKGIAKSRKSGS | |
| pCoV269 | RBD-RBD-Ferritin | His8-3c-M3.7RBD-GSGGSG- | HHHHHHHHGPLEVLFQGPEGVECDFSPLLSGTPPQVYNFKRLVFTNCNYNLTKLLSLFSVNDFTCSQI | 48 |
| | | RBD(131)-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVAEDGDYYRKQLSPLEGGGWLVASGSTVAMTEQL | |
| | | | QMGFGITVQNGTDTNSVCPKGSGGSGNITNLCPFGEVFNATRFASVYAWNRKRISNCVADYSVLYNS | |
| | | | ASFSTFKCYGVSPTKLNDLCFTNVYADSFVIRGDEVRQIAPGQTGKIADYNYKLPDDFTGCVIAWNSN | |
| | | | NLDSKVGGNYNYLRRLFRKSNLKPFERDISTEIYQAGSTPCNGVEGFNCYFPLQSYGFQPTNGVGYQ | |
| | | | PYRVVVLSFENKSAPATVCGPGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoV270 | RBD-RBD-Ferritin | His8-3c-M3.7RBD-GSGGSG- | HHHHHHHHGPLEVLFQGPEGVECDFSPLLSGTPPQVYNFKRLVFTNCNYNLTKLLSLFSVNDFTCSQI | 49 |
| | | RBD(127)-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVAEDGDYYRKQLSPLEGGGWLVASGSTVAMTEQL | |
| | | | QMGFGITVQNGTDTNSVCPKGSGGSGNITNLCPFGEVFNATRFASVYAWNRKRISNCVADYSVLYNS | |
| | | | ASFSTFKCYGVSPTKLNDLCFTNVYADSFVIRGDEVRQIAPGQTGKIADYNYKLPDDFTGCVIAWNSN | |
| | | | NLDSKVGGNYNYLYRLFRKSNLKPFERDISTEIYQAGSTPCNGVEGFNCYAPLQSYGFQPTNGVGYQ | |
| | | | PYRVVVLSFENKSAPATVCGPGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoV272 | SpFN | A701V in Omicron SpFN | SSQCVNLTTRTQLPPAYTNSFTRGVYYPDKVFRSSVLHSTQDLFLPFFSNVTWFHVISGTNGTKRFDN | 50 |
| | | plasmid | PVLPFNDGVYFASIEKSNIIRGWIFGTTLDSKTQSLLIVNNATNVVIKVCEFQFCNDPFLDHKNNKSWME | |
| | | | SEFRVYSSANNCTFEYVSQPFLMDLEGKQGNFKNLREFVFKNIDGYFKIYSKHTPIIVREPEDLPQGFS | |
| | | | ALEPLVDLPIGINITRFQTLLALHRSYLTPGDSSSGWTAGAAAYYVGYLQPRTFLLKYNENGTITDAVDC | |
| | | | ALDPLSETKCTLKSFTVEKGIYQTSNFRVQPTESIVRFPNITNLCPFDEVFNATRFASVYAWNRKRISNC | |
| | | | VADYSVLYNLAPFFTFKCYGVSPTKLNDLCFTNVYADSFVIRGDEVRQIAPGQTGNIADYNYKLPDDFT | |
| | | | GCVIAWNSNKLDSKVSGNYNYLYRLFRKSNLKPFERDISTEIYQAGNKPCNGVAGFNCYFPLRSYSFR | |
| | | | PTYGVGHQPYRVVVLSFELLHAPATVCGPKKSTNLVKNKCVNFNFNGLKGTGVLTESNKKFLPFQQF | |
| | | | GRDIADTTDAVRDPQTLEILDITPCSFGGVSVITPGTNTSNQVAVLYQGVNCTEVPVAIHADQLTPTWR | |
| | | | VYSTGSNVFQTRAGCLIGAEYVNNSYECDIPIGAGICASYQTQTKSHGSASSVASQSIIAYTMSLGVEN | |
| | | | SVAYSNNSIAIPTNFTISVTTEILPVSMTKTSVDCTMYICGDSTECSNLLLQYGSFCTQLKRALTGIAVEQ | |
| | | | DKNTQEVFAQVKQIYKTPPIKYFGGFNFSQILPDPSKPSKRSFIEDLLFNKVTLADAGFIKQYGDCLGDI | |
| | | | AARDLICAQKFKGLTVLPPLLTDEMIAQYTSALLAGTITSGWTFGAGAALQIPFAMQMAYRFNGIGVTQ | |
| | | | NVLYENQKLIANQFNSAIGKIQDSLSSTASALGKLQDVVNHNAQALNTLVKQLSSKFGAISSVLNDIFSR | |
| | | | LDPPEAEVQIDRLITGRLQSLQTYVTQQLIRAAEIRASANLAATKMSECVLGQSKRVDFCGKGYHLMSF | |
| | | | PQSAPHGVVFLHVTYVPAQEKNFTTAPAICHDGKAHFPREGVFVSNGTHWFVTQRNFYEPQIITTDNT | |
| | | | FVSGNCDVVIGIVNNTVYDPLQSELDSIKEELDKIHKNGSGGSGDIIKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| pCoV273 | R2-SD-Ferritin | His8-3c-R2-SD-FN where | HHHHHHHHGPLEVLFQGPNITNLCPFGEVFNATKFPSVYAWERKKISNCVADYSVLYNSTFFSTFKCY | 51 |
| | | SARS-1 RBD is added | GVSATKLNDLCFSNVYADSFVVKGDDVRQIAPGQTGVIADYNYKLPDDFMGCVLAWNTRNIDATSTG | |
| | | | NYNYKYRYLRHGKLRPFERDISNVPFSPDGKPCTPPALNCYWPLNDYGFYTTTGIGYQPYRVVVLSFE | |
| | | | LLNAPATVCGPGSGSCALDPLSETKCTLKSFTVEKGIYQTSNFRVQPTESIVRFPNITNLCPFGEVFNA | |
| | | | TRFASVYAWNRKRISNCVADYSVLYNSASFSTFKCYGVSPTKLNDLCFTNVYADSFVIRGDEVRQIAP | |
| | | | GQTGKIADYNYKLPDDFTGCVIAWNSNNLDSKVGGNYNYLYRLFRKSNLKPFERDISTEIYQAGSTPC | |
| | | | NGVEGFNCYFPLQSYGFQPTNGVGYQPYRVVVLSFELLHAPATVCGPKKSTNLVKNKCVNFNFNGLT | |
| | | | GTGVLTESNKKFLPFQQFGRDIADTTDAVRDPQTLEILDITPCSFGGVSVITPGTNTSNQVAVLYQDVN | |
| | | | CTEVPVAIHADQLTPTWRVYSTGSNVFQTRAGCLIGAEHVNNSYECDIPIGAGICASYQTGGSQSIIAY | |
| | | | TGSGGGGESQVRQQFSKDIEKLLNEQVNKEMQSSNLYMSMSSWCYTHSLDGAGLFLFDHAAEEYEH | |
| | | | AKKLIIFLNENNVPVQLTSISAPEHKFEGLTQIFQKAYEHEQHISESINNIVDHAIKSKDHATFNFLQWYV | |
| | | | AEQHEEEVLFKDILDKIELIGNENHGLYLADQYVKGIAKSRKSGS | |
| pCoV274 | R-S1-Ferritin | His8-3c-R-S1-FN where | HHHHHHHHGPLEVLFQGPNITNLCPFGEVFNATKFPSVYAWERKKISNCVADYSVLYNSTFFSTFKCY | 52 |
| | | SARS-1 RBD is added | GVSATKLNDLCFSNVYADSFVVKGDDVRQIAPGQTGVIADYNYKLPDDFMGCVLAWNTRNIDATSTG | |
| | | | NYNYKYRYLRHGKLRPFERDISNVPFSPDGKPCTPPALNCYWPLNDYGFYTTTGIGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoV275 | R2-SD-S2-FN (NTD | His8-3c-R2-SD-S2-FN where | HHHHHHHHGPLEVLFQGPNITNLCPFGEVFNATKFPSVYAWERKKISNCVADYSVLYNSTFFSTFKCY | 53 |
| | replaced by a | SARS-1 RBD is added | GVSATKLNDLCFSNVYADSFVVKGDDVRQIAPGQTGVIADYNYKLPDDFMGCVLAWNTRNIDATSTG | |
| | second RBD) | (Hexapro Version) | NYNYKYRYLRHGKLRPFERDISNVPFSPDGKPCTPPALNCYWPLNDYGFYTTTGIGYQPYRVVVLSFE | |
| | | | LLNAPATVCGPGSGSCALDPLSETKCTLKSFTVEKGIYQTSNFRVQPTESIVRFPNITNLCPFGEVFNA | |
| | | | TRFASVYAWNRKRISNCVADYSVLYNSASFSTFKCYGVSPTKLNDLCFTNVYADSFVIRGDEVRQIAP | |
| | | | GQTGKIADYNYKLPDDFTGCVIAWNSNNLDSKVGGNYNYLYRLFRKSNLKPFERDISTEIYQAGSTPC | |
| | | | NGVEGFNCYFPLQSYGFQPTNGVGYQPYRVVVLSFELLHAPATVCGPKKSTNLVKNKCVNFNFNGLT | |
| | | | GTGVLTESNKKFLPFQQFGRDIADTTDAVRDPQTLEILDITPCSFGGVSVITPGTNTSNQVAVLYQDVN | |
| | | | CTEVPVAIHADQLTPTWRVYSTGSNVFQTRAGCLIGAEHVNNSYECDIPIGAGICASYQTQTNSPGSA | |
| | | | SSVASQSIIAYTMSLGAENSVAYSNNSIAIPTNFTISVTTEILPVSMTKTSVDCTMYICGDSTECSNLLLQ | |
| | | | YGSFCTQLNRALTGIAVEQDKNTQEVFAQVKQIYKTPPIKDFGGFNFSQILPDPSKPSKRSPIEDLLFNK | |
| | | | VTLADAGFIKQYGDCLGDIAARDLICAQKFNGLTVLPPLLTDEMIAQYTSALLAGTITSGWTFGAGPALQ | |
| | | | IPFPMQMAYRFNGIGVTQNVLYENQKLIANQFNSAIGKIQDSLSSTPSALGKLQDVVNQNAQALNTLVK | |
| | | | QLSSNFGAISSVLNDILSRLDPPEAEVQIDRLITGRLQSLQTYVTQQLIRAAEIRASANLAATKMSECVLG | |
| | | | QSKRVDFCGKGYHLMSFPQSAPHGVVFLHVTYVPAQEKNFTTAPAICHDGKAHFPREGVFVSNGTH | |
| | | | WFVTQRNFYEPQIITTDNTFVSGNCDWVIGIVNNTVYDPLQSELDSIKEELDKIHKNGSGGSGDIIKLLNE | |
| | | | QVNKEMQSSNLYMSMSSWCYTHSLDGAGLFLFDHAAEEYEHAKKLIIFLNENNVPVQLTSISAPEHKF | |
| | | | EGLTQIFQKAYEHEQHISESINNIVDHAIKSKDHATFNFLQWYVAEQHEEEVLFKDILDKIELIGNENHGL | |
| | | | YLADQYVKGIAKSRKSGS | |
| pCoV276 | RBD-Spike- | His8-3c-R-SpFN where | HHHHHHHHGPLEVLFQGPNITNLCPFGEVFNATKFPSVYAWERKKISNCVADYSVLYNSTFFSTFKCY | 54 |
| | Ferritin | SARS-1 RBD is added | GVSATKLNDLCFSNVYADSFVVKGDDVRQIAPGQTGVIADYNYKLPDDFMGCVLAWNTRNIDATSTG | |
| | | (Hexapro Version) | NYNYKYRYLRHGKLRPFERDISNVPFSPDGKPCTPPALNCYWPLNDYGFYTTTGIGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTQTN | |
| | | | SPGSASSVASQSIIAYTMSLGAENSVAYSNNSIAIPTNFTISVTTEILPVSMTKTSVDCTMYICGDSTECS | |
| | | | NLLLQYGSFCTQLNRALTGIAVEQDKNTQEVFAQVKQIYKTPPIKDFGGFNFSQILPDPSKPSKRSPIED | |
| | | | LLFNKVTLADAGFIKQYGDCLGDIAARDLICAQKFNGLTVLPPLLTDEMIAQYTSALLAGTITSGWTFGA | |
| | | | GPALQIPFPMQMAYRFNGIGVTQNVLYENQKLIANQFNSAIGKIQDSLSSTPSALGKLQDVVNQNAQAL | |
| | | | NTLVKQLSSNFGAISSVLNDILSRLDPPEAEVQIDRLITGRLQSLQTYVTQQLIRAAEIRASANLAATKMS | |
| | | | ECVLGQSKRVDFCGKGYHLMSFPQSAPHGVVFLHVTYVPAQEKNFTTAPAICHDGKAHFPREGVFVS | |
| | | | NGTHWFVTQRNFYEPQIITTDNTFVSGNCDWVIGIVNNTVYDPLQSELDSIKEELDKIHKNGSGGSGDII | |
| | | | KLLNEQVNKEMQSSNLYMSMSSWCYTHSLDGAGLFLFDHAAEEYEHAKKLIIFLNENNVPVQLTSISAP | |
| | | | EHKFEGLTQIFQKAYEHEQHISESINNIVDHAIKSKDHATFNFLQWYVAEQHEEEVLFKDILDKIELIGNE | |
| | | | NHGLYLADQYVKGIAKSRKSGS | |
| pCoV277 | RBD-RBD-NTD- | His8-3c-RRN-FN where | HHHHHHHHGPLEVLFQGPNITNLCPFGEVFNATKFPSVYAWERKKISNCVADYSVLYNSTFFSTFKCY | 55 |
| | Ferritin | SARS-1 RBD is added to | GVSATKLNDLCFSNVYADSFVVKGDDVRQIAPGQTGVIADYNYKLPDDFMGCVLAWNTRNIDATSTG | |
| | | pCoV146 | NYNYKYRYLRHGKLRPFERDISNVPFSPDGKPCTPPALNCYWPLNDYGFYTTTGIGYQPYRVVVLSFE | |
| | | | LLNAPATVCGPGSGGNITNLCPFGEVFNATRFASVYAWNRKRISNCVADYSVLYNSASFSTFKCYGVS | |
| | | | PTKLNDLCFTNVYADSFVIRGDEVRQIAPGQTGKIADYNYKLPDDFTGCVIAWNSNNLDSKVGGNYNY | |
| | | | LRRLFRKSNLKPFERDISTEIYQAGSTPCNGVEGFNCYFPLQSYGFQPTNGVGYQPYRVVVLSFENKS | |
| | | | APATVCGPGSGGSGSSQCVNLTTRTQLPPAYTNSFTRGVYYPDKVFRSSVLHSTQDLFLPFFSNVTW | |
| | | | FHAIHVSGTNGTKRFDNPVLPFNDGVYFASTEKSNIIRGWIFGTTLDSKTQSLLIVNNATNVVIKVCEFQ | |
| | | | FCNDPFLGVYYHKNNKSWMESEFRVYSSANNCTFEYVSQPFLMDLEGKQGNFKNLREFVFKNIDGYF | |
| | | | KIYSKHTPINLVRDLPQGFSALEPLVDLPIGINITRFQTLLALHRSYLTPGDSSSGWTAGAAAYYVGYLQ | |
| | | | PRTFLLKYNENGTITDAVDCALDPLSETKCTLGSGGGGESQVRQQFSKDIEKLLNEQVNKEMQSSNLY | |
| | | | MSMSSWCYTHSLDGAGLFLFDHAAEEYEHAKKLIIFLNENNVPVQLTSISAPEHKFEGLTQIFQKAYEH | |
| | | | EQHISESINNIVDHAIKSKDHATFNFLQWYVAEQHEEEVLFKDILDKIELIGNENHGLYLADQYVKGIAKS | |
| | | | RKSGS | |
| pCoV278 | RBD-NTD-RBD-NTD- | His8-3c-RNRN-FN where | HHHHHHHHGPLEVLFQGPNITNLCPFGEVFNATKFPSVYAWERKKISNCVADYSVLYNSTFFSTFKCY | 56 |
| | Ferritin | SARS-1 RBD and NTD are | GVSATKLNDLCFSNVYADSFVVKGDDVRQIAPGQTGVIADYNYKLPDDFMGCVLAWNTRNIDATSTG | |
| | | added to pCoV146 | NYNYKYRYLRHGKLRPFERDISNVPFSPDGKPCTPPALNCYWPLNDYGFYTTTGIGYQPYRVVVLSFE | |
| | | | LLNAPATVCGPGSGGLDRCTTFDDVQAPNYTQHTSSMRGVYYPDEIFRSDTLYLTQDLFLPFYSNVTG | |
| | | | FHTINHTFGNPVIPFKDGIYFAATEKSNVVRGWVFGSTMNNKSQSVIIINNSTNWVIRACNFELCDNPFF | |
| | | | AVSKPMGTQTHTMIFDNAFNCTFEYISDAFSLDVSEKSGNFKHLREFVFKNKDGFLYVYKGYQPIDVV | |
| | | | RDLPSGFNTLKPIFKLPLGINITNFRAILTAFSPAQDIWGTSAAAYFVGYLKPTTFMLKYDENGTITDAVD | |
| | | | CSQNPLAELKCSVGSGGSGNITNLCPFGEVFNATRFASVYAWNRKRISNCVADYSVLYNSASFSTFKC | |
| | | | YGVSPTKLNDLCFTNVYADSFVIRGDEVRQIAPGQTGKIADYNYKLPDDFTGCVIAWNSNNLDSKVGG | |
| | | | NYNYLRRLFRKSNLKPFERDISTEIYQAGSTPCNGVEGFNCYFPLQSYGFQPTNGVGYQPYRVVVLSF | |
| | | | ENKSAPATVCGPGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLGSGGGGESQVRQQFSKDIEKLLNEQVNKEMQ | |
| | | | SSNLYMSMSSWCYTHSLDGAGLFLFDHAAEEYEHAKKLIIFLNENNVPVQLTSISAPEHKFEGLTQIFQ | |
| | | | KAYEHEQHISESINNIVDHAIKSKDHATFNFLQWYVAEQHEEEVLFKDILDKIELIGNENHGLYLADQYV | |
| | | | KGIAKSRKSGS | |
| pCoV287 | RBD inserted into | His8-3c-R2N-FN-70 where | HHHHHHHHGPLEVLFQGPNITNLCPFGEVFNATRFASVYAWNRKRISNCVADYSVLYNSASFSTFKCY | 57 |
| | NTD | SARS-1 RBD is inserted into | GVSPTKLNDLCFTNVYADSFVIRGDEVRQIAPGQTGKIADYNYKLPDDFTGCVIAWNSNNLDSKVGGN | |
| | loop-Ferritin | the NTD (loop at 70) in | YNYLRRLFRKSNLKPFERDISTEIYQAGSTPCNGVEGFNCYFPLQSYGFQPTNGVGYQPYRVVVLSFE | |
| | | pCoV146 | NKSAPATVCGPGSGGSGSSQCVNLTTRTQLPPAYTNSFTRGVYYPDKVFRSSVLHSTQDLFLPFFSN | |
| | | | VTWFHAIHVGSGGSGNITNLCPFGEVFNATKFPSVYAWERKKISNCVADYSVLYNSTFFSTFKCYGVS | |
| | | | ATKLNDLCFSNVYADSFVVKGDDVRQIAPGQTGVIADYNYKLPDDFMGCVLAWNTRNIDATSTGNYN | |
| | | | YKYRYLRHGKLRPFERDISNVPFSPDGKPCTPPALNCYWPLNDYGFYTTTGIGYQPYRVVVLSFELLN | |
| | | | APATVCGPGSGGSGSGTNGTKRFDNPVLPFNDGVYFASTEKSNIIRGWIFGTTLDSKTQSLLIVNNAT | |
| | | | NVVIKVCEFQFCNDPFLGVYYHKNNKSWMESEFRVYSSANNCTFEYVSQPFLMDLEGKQGNFKNLR | |
| | | | EFVFKNIDGYFKIYSKHTPINLVRDLPQGFSALEPLVDLPIGINITRFQTLLALHRSYLTPGDSSSGWTAG | |
| | | | AAAYYVGYLQPRTFLLKYNENGTITDAVDCALDPLSETKCTLGSGGGGESQVRQQFSKDIEKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoV288 | RBD inserted into | His8-3c-R2N-FN-148 where | HHHHHHHHGPLEVLFQGPNITNLCPFGEVFNATRFASVYAWNRKRISNCVADYSVLYNSASFSTFKCY | 58 |
| | NTD | SARS-1 RBD is inserted into | GVSPTKLNDLCFTNVYADSFVIRGDEVRQIAPGQTGKIADYNYKLPDDFTGCVIAWNSNNLDSKVGGN | |
| | loop-Ferritin | the NTD (loop at 148) in | YNYLRRLFRKSNLKPFERDISTEIYQAGSTPCNGVEGFNCYFPLQSYGFQPTNGVGYQPYRVVVLSFE | |
| | | pCoV146 | NKSAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNGSGGSGNITNLCPFGEVFNATKFPSVYAWERKKISNCVADYSVLYNSTFFS | |
| | | | TFKCYGVSATKLNDLCFSNVYADSFVVKGDDVRQIAPGQTGVIADYNYKLPDDFMGCVLAWNTRNIDA | |
| | | | TSTGNYNYKYRYLRHGKLRPFERDISNVPFSPDGKPCTPPALNCYWPLNDYGFYTTTGIGYQPYRVV | |
| | | | VLSFELLNAPATVCGPGSGGSGNKSWMESEFRVYSSANNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLGSGGGGESQVRQQFSKDIEKLLNEQVNK | |
| | | | EMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoV289 | RBD inserted into | His8-3c-R2N-FN-166 where | HHHHHHHHGPLEVLFQGPNITNLCPFGEVFNATRFASVYAWNRKRISNCVADYSVLYNSASFSTFKCY | 59 |
| | NTD | SARS-1 RBD is inserted into | GVSPTKLNDLCFTNVYADSFVIRGDEVRQIAPGQTGKIADYNYKLPDDFTGCVIAWNSNNLDSKVGGN | |
| | loop-Ferritin | the NTD (loop at 166) in | YNYLRRLFRKSNLKPFERDISTEIYQAGSTPCNGVEGFNCYFPLQSYGFQPTNGVGYQPYRVVVLSFE | |
| | | pCoV146 | NKSAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANGSGGSGNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPGSGGSGNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLGSGGGGESQVRQQFSKDIEKLLNEQVNK | |
| | | | EMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoV290 | RBD inserted into | His8-3c-R-SpFN where MERS | HHHHHHHHGPLEVLFQGPEGVECDFSPLLSGTPPQVYNFKRLVFTNCNYNLTKLLSLFSVNDFTCSQI | 60 |
| | NTD | (M3) RBD is added (similar | SPAAIASNCYSSLILDYFSYPLSMKSDLSVSSAGPISQFNYKQSFSNPTCLILATVPHNLTTITKPLKYSYI | |
| | loop-Ferritin | to pCoV276 but SARS1 RBD | NKCSRLLSDDRTEVPQLVNANQYSPCVSIVPSTVWEDGDYYRKQLSPLEGGGWLVASGSTVAMTEQ | |
| | | is replaced with M3 RBD) | LQMGFGITVQNGTDTNSVCPKGSGGSGSSQCVNLTTRTQLPPAYTNSFTRGVYYPDKVFRSSVLHST | |
| | | (Hexapro Version) | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLKSFTVEKGIYQTSNFRVQPTESIV | |
| | | | RFPNITNLCPFGEVFNATRFASVYAWNRKRISNCVADYSVLYNSASFSTFKCYGVSPTKLNDLCFTNV | |
| | | | YADSFVIRGDEVRQIAPGQTGKIADYNYKLPDDFTGCVIAWNSNNLDSKVGGNYNYLYRLFRKSNLKP | |
| | | | FERDISTEIYQAGSTPCNGVEGFNCYFPLQSYGFQPTNGVGYQPYRVVVLSFELLHAPATVCGPKKST | |
| | | | NLVKNKCVNFNFNGLTGTGVLTESNKKFLPFQQFGRDIADTTDAVRDPQTLEILDITPCSFGGVSVITP | |
| | | | GTNTSNQVAVLYQDVNCTEVPVAIHADQLTPTWRVYSTGSNVFQTRAGCLIGAEHVNNSYECDIPIGA | |
| | | | GICASYQTQTNSPGSASSVASQSIIAYTMSLGAENSVAYSNNSIAIPTNFTISVTTEILPVSMTKTSVDCT | |
| | | | MYICGDSTECSNLLLQYGSFCTQLNRALTGIAVEQDKNTQEVFAQVKQIYKTPPIKDFGGFNFSQILPD | |
| | | | PSKPSKRSPIEDLLFNKVTLADAGFIKQYGDCLGDIAARDLICAQKFNGLTVLPPLLTDEMIAQYTSALLA | |
| | | | GTITSGWTFGAGPALQIPFPMQMAYRFNGIGVTQNVLYENQKLIANQFNSAIGKIQDSLSSTPSALGKL | |
| | | | QDVVNQNAQALNTLVKQLSSNFGAISSVLNDILSRLDPPEAEVQIDRLITGRLQSLQTYVTQQLIRAAEI | |
| | | | RASANLAATKMSECVLGQSKRVDFCGKGYHLMSFPQSAPHGVVFLHVTYVPAQEKNFTTAPAICHDG | |
| | | | KAHFPREGVFVSNGTHWFVTQRNFYEPQIITTDNTFVSGNCDVVIGIVNNTVYDPLQSELDSIKEELDKI | |
| | | | HKNGSGGSGDIIKLLNEQVNKEMQSSNLYMSMSSWCYTHSLDGAGLFLFDHAAEEYEHAKKLIIFLNE | |
| | | | NNVPVQLTSISAPEHKFEGLTQIFQKAYEHEQHISESINNIVDHAIKSKDHATFNFLQWYVAEQHEEEVL | |
| | | | FKDILDKIELIGNENHGLYLADQYVKGIAKSRKSGS | |
| pCoV291 | RBD inserted into | His8-3c-RR-SpFN where MERS | HHHHHHHHGPLEVLFQGPEGVECDFSPLLSGTPPQVYNFKRLVFTNCNYNLTKLLSLFSVNDFTCSQI | 61 |
| | NTD | (M3) RBD is added in | SPAAIASNCYSSLILDYFSYPLSMKSDLSVSSAGPISQFNYKQSFSNPTCLILATVPHNLTTITKPLKYSYI | |
| | loop-Ferritin | addition to the SARS1 RBD | NKCSRLLSDDRTEVPQLVNANQYSPCVSIVPSTVWEDGDYYRKQLSPLEGGGWLVASGSTVAMTEQ | |
| | | that's already in pCoV276) | LQMGFGITVQNGTDTNSVCPKGSGGSGNITNLCPFGEVFNATKFPSVYAWERKKISNCVADYSVLYN | |
| | | (Hexapro Version) | STFFSTFKCYGVSATKLNDLCFSNVYADSFVVKGDDVRQIAPGQTGVIADYNYKLPDDFMGCVLAWN | |
| | | | TRNIDATSTGNYNYKYRYLRHGKLRPFERDISNVPFSPDGKPCTPPALNCYWPLNDYGFYTTTGIGYQ | |
| | | | PYRVVVLSFELLNAPATVCGPGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLKSFTVEKGIYQTSNFRVQPTESIV | |
| | | | RFPNITNLCPFGEVFNATRFASVYAWNRKRISNCVADYSVLYNSASFSTFKCYGVSPTKLNDLCFTNV | |
| | | | YADSFVIRGDEVRQIAPGQTGKIADYNYKLPDDFTGCVIAWNSNNLDSKVGGNYNYLYRLFRKSNLKP | |
| | | | FERDISTEIYQAGSTPCNGVEGFNCYFPLQSYGFQPTNGVGYQPYRVVVLSFELLHAPATVCGPKKST | |
| | | | NLVKNKCVNFNFNGLTGTGVLTESNKKFLPFQQFGRDIADTTDAVRDPQTLEILDITPCSFGGVSVITP | |
| | | | GTNTSNQVAVLYQDVNCTEVPVAIHADQLTPTWRVYSTGSNVFQTRAGCLIGAEHVNNSYECDIPIGA | |
| | | | GICASYQTQTNSPGSASSVASQSIIAYTMSLGAENSVAYSNNSIAIPTNFTISVTTEILPVSMTKTSVDCT | |
| | | | MYICGDSTECSNLLLQYGSFCTQLNRALTGIAVEQDKNTQEVFAQVKQIYKTPPIKDFGGFNFSQILPD | |
| | | | PSKPSKRSPIEDLLFNKVTLADAGFIKQYGDCLGDIAARDLICAQKFNGLTVLPPLLTDEMIAQYTSALLA | |
| | | | GTITSGWTFGAGPALQIPFPMQMAYRFNGIGVTQNVLYENQKLIANQFNSAIGKIQDSLSSTPSALGKL | |
| | | | QDVVNQNAQALNTLVKQLSSNFGAISSVLNDILSRLDPPEAEVQIDRLITGRLQSLQTYVTQQLIRAAEI | |
| | | | RASANLAATKMSECVLGQSKRVDFCGKGYHLMSFPQSAPHGVVFLHVTYVPAQEKNFTTAPAICHDG | |
| | | | KAHFPREGVFVSNGTHWFVTQRNFYEPQIITTDNTFVSGNCDVVIGIVNNTVYDPLQSELDSIKEELDKI | |
| | | | HKNGSGGSGDIIKLLNEQVNKEMQSSNLYMSMSSWCYTHSLDGAGLFLFDHAAEEYEHAKKLIIFLNE | |
| | | | NNVPVQLTSISAPEHKFEGLTQIFQKAYEHEQHISESINNIVDHAIKSKDHATFNFLQWYVAEQHEEEVL | |
| | | | FKDILDKIELIGNENHGLYLADQYVKGIAKSRKSGS | |
| pCoV292 | RBD insert | RBD-His6 from Omicron | NITNLCPFDEVFNATRFASVYAWNRKRISNCVADYSVLYNFAPFFAFKCYGVSPTKLNDLCFTNVYADS | 62 |
| | | BA.2.12.1 | FVIRGNEVSQIAPGQTGNIADYNYKLPDDFTGCVIAWNSNKLDSKVGGNYNYQYRLFRKSNLKPFERD | |
| | | | ISTEIYQAGNKPCNGVAGFNCYFPLRSYGFRPTYGVGHQPYRVVVLSFELLHAPATVCGPGSHHHHH | |
| | | | H | |
| pCoV294 | SpFN | VFLIP version of SpFN: | SSQCVNLTTRTQLPPAYTNSFTRGVYYPDKVFRSSVLHSTQDLFLPFFSNVTWFHAIHVSGTNGTKRF | 63 |
| | | PrefLead-Spike-FL2-Y707C- | DNPVLPFNDGVYFASTEKSNIIRGWIFGTTLDSKTQSLLIVNNATNVVIKVCEFQFCNDPFLGVYYHKNN | |
| | | T883C-P986K-Ferritin | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATRFASVYAWNRK | |
| | | | RISNCVADYSVLYNSASFSTFKCYGVSPTKLNDLCFTNVYADSFVIRGDEVRQIAPGQTGKIADYNYKL | |
| | | | PDDFTGCVIAWNSNNLDSKVGGNYNYLYRLFRKSNLKPFERDISTEIYQAGSTPCNGVEGFNCYFPLQ | |
| | | | SYGFQPTNGVGYQPYRVVVLSFELLHAPATVCGPKKSTNLVKNKCVNFNFNGLTGTGVLTESNKKFL | |
| | | | PFQQFGRDIADTTDAVRDPQTLEILDITPCSFGGVSVITPGTNTSNQVAVLYQDVNCTEVPVAIHADQL | |
| | | | TPTWRVYSTGSNVFQTRAGCLIGAEHVNNSYECDIPIGAGICASYQGGGSGGGSSIIAYTMSLGAENS | |
| | | | VACSNNSIAIPTNFTISVTTEILPVSMTKTSVDCTMYICGDSTECSNLLLQYGSFCTQLNRALTGIAVEQD | |
| | | | KNTQEVFAQVKQIYKTPPIKDFGGFNFSQILPDPSKPSKRSPIEDLLFNKVTLADAGFIKQYGDCLGDIA | |
| | | | ARDLICAQKFNGLTVLPPLLTDEMIAQYTSALLAGTICSGWTFGAGPALQIPFPMQMAYRFNGIGVTQN | |
| | | | VLYENQKLIANQFNSAIGKIQDSLSSTPSALGKLQDVVNQNAQALNTLVKQLSSNFGAISSVLNDILSRL | |
| | | | DKPEAEVQIDRLITGRLQSLQTYVTQQLIRAAEIRASANLAATKMSECVLGQSKRVDFCGKGYHLMSFP | |
| | | | QSAPHGVVFLHVTYVPAQEKNFTTAPAICHDGKAHFPREGVFVSNGTHWFVTQRNFYEPQIITTDNTF | |
| | | | VSGNCDVVIGIVNNTVYDPLQSELDSIKEELDKIHKNGSGGSGDIIKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESI | |
| | | | NNIVDHAIKSKDHATFNFLQWYVAEQHEEEVLFKDILDKIELIGNENHGLYLADQYVKGIAKSRKSGS | |
| pCoV295 | RBD-Spike- | VFLIP version of R-SpFN | HHHHHHHHGPLEVLFQGPNITNLCPFGEVFNATKFPSVYAWERKKISNCVADYSVLYNSTFFSTFKCY | 64 |
| | Ferritin | with SARS1 RBD added: His8- | GVSATKLNDLCFSNVYADSFVVKGDDVRQIAPGQTGVIADYNYKLPDDFMGCVLAWNTRNIDATSTG | |
| | | 3c-PrefLead-Spike-FL2- | NYNYKYRYLRHGKLRPFERDISNVPFSPDGKPCTPPALNCYWPLNDYGFYTTTGIGYQPYRVVVLSFE | |
| | | Y707C-T883C-P986K-Ferritin | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQGGGS | |
| | | | GGGSSIIAYTMSLGAENSVACSNNSIAIPTNFTISVTTEILPVSMTKTSVDCTMYICGDSTECSNLLLQYG | |
| | | | SFCTQLNRALTGIAVEQDKNTQEVFAQVKQIYKTPPIKDFGGFNFSQILPDPSKPSKRSPIEDLLFNKVT | |
| | | | LADAGFIKQYGDCLGDIAARDLICAQKFNGLTVLPPLLTDEMIAQYTSALLAGTICSGWTFGAGPALQIP | |
| | | | FPMQMAYRFNGIGVTQNVLYENQKLIANQFNSAIGKIQDSLSSTPSALGKLQDVVNQNAQALNTLVKQ | |
| | | | LSSNFGAISSVLNDILSRLDKPEAEVQIDRLITGRLQSLQTYVTQQLIRAAEIRASANLAATKMSECVLGQ | |
| | | | SKRVDFCGKGYHLMSFPQSAPHGVVFLHVTYVPAQEKNFTTAPAICHDGKAHFPREGVFVSNGTHW | |
| | | | FVTQRNFYEPQIITTDNTFVSGNCDVVIGIVNNTVYDPLQSELDSIKEELDKIHKNGSGGSGDIIKLLNEQ | |
| | | | VNKEMQSSNLYMSMSSWCYTHSLDGAGLFLFDHAAEEYEHAKKLIIFLNENNVPVQLTSISAPEHKFE | |
| | | | GLTQIFQKAYEHEQHISESINNIVDHAIKSKDHATFNFLQWYVAEQHEEEVLFKDILDKIELIGNENHGLY | |
| | | | LADQYVKGIAKSRKSGS | |
| pCoV298 | mosaic SpFN | SpFN with SARS1 RBD | SSQCVNLTTRTQLPPAYTNSFTRGVYYPDKVFRSSVLHSTQDLFLPFFSNVTWFHAIHVSGTNGTKRF | 65 |
| | | replacing the original RBD | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATKFPSVYAWERK | |
| | | | KISNCVADYSVLYNSTFFSTFKCYGVSATKLNDLCFSNVYADSFVVKGDDVRQIAPGQTGVIADYNYKL | |
| | | | PDDFMGCVLAWNTRNIDATSTGNYNYKYRYLRHGKLRPFERDISNVPFSPDGKPCTPPALNCYWPLN | |
| | | | DYGFYTTTGIGYQPYRVVVLSFELLNAPATVCGPKKSTNLVKNKCVNFNFNGLTGTGVLTESNKKFLP | |
| | | | FQQFGRDIADTTDAVRDPQTLEILDITPCSFGGVSVITPGTNTSNQVAVLYQDVNCTEVPVAIHADQLT | |
| | | | PTWRVYSTGSNVFQTRAGCLIGAEHVNNSYECDIPIGAGICASYQTQTNSPGSASSVASQSIIAYTMSL | |
| | | | GAENSVAYSNNSIAIPTNFTISVTTEILPVSMTKTSVDCTMYICGDSTECSNLLLQYGSFCTQLNRALTGI | |
| | | | AVEQDKNTQEVFAQVKQIYKTPPIKDFGGFNFSQILPDPSKPSKRSFIEDLLFNKVTLADAGFIKQYGDC | |
| | | | LGDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoV300 | RBD-Spike- | His8-3c-R-SpFN with | HHHHHHHHGPLEVLFQGPNITNLCPFGEVFNATKFPSVYAWERKKISNCVADYSVLYNSTFFSTFKCY | 66 |
| | Ferritin | original SpFN where | GVSATKLNDLCFSNVYADSFVVKGDDVRQIAPGQTGVIADYNYKLPDDFMGCVLAWNTRNIDATSTG | |
| | | SARS-1 RBD is added | NYNYKYRYLRHGKLRPFERDISNVPFSPDGKPCTPPALNCYWPLNDYGFYTTTGIGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTQTN | |
| | | | SPGSASSVASQSIIAYTMSLGAENSVAYSNNSIAIPTNFTISVTTEILPVSMTKTSVDCTMYICGDSTECS | |
| | | | NLLLQYGSFCTQLNRALTGIAVEQDKNTQEVFAQVKQIYKTPPIKDFGGFNFSQILPDPSKPSKRSFIED | |
| | | | LLFNKVTLADAGFIKQYGDCLGDIAARDLICAQKFNGLTVLPPLLTDEMIAQYTSALLAGTITSGWTFGA | |
| | | | GAALQIPFAMQMAYRFNGIGVTQNVLYENQKLIANQFNSAIGKIQDSLSSTASALGKLQDVVNQNAQAL | |
| | | | NTLVKQLSSNFGAISSVLNDILSRLDPPEAEVQIDRLITGRLQSLQTYVTQQLIRAAEIRASANLAATKMS | |
| | | | ECVLGQSKRVDFCGKGYHLMSFPQSAPHGVVFLHVTYVPAQEKNFTTAPAICHDGKAHFPREGVFVS | |
| | | | NGTHWFVTQRNFYEPQIITTDNTFVSGNCDVVIGIVNNTVYDPLQSELDSIKEELDKIHKNGSGGSGDII | |
| | | | KLLNEQVNKEMQSSNLYMSMSSWCYTHSLDGAGLFLFDHAAEEYEHAKKLIIFLNENNVPVQLTSISAP | |
| | | | EHKFEGLTQIFQKAYEHEQHISESINNIVDHAIKSKDHATFNFLQWYVAEQHEEEVLFKDILDKIELIGNE | |
| | | | NHGLYLADQYVKGIAKSRKSGS | |
| pCoV301 | RBD-RBD-Ferritin | His8-3c-SARS1RBD(131like)- | HHHHHHHHGPLEVLFQGPNITNLCPFGEVFNATKFPSVYAWERKKISNCVADYSVLYNSTFFSTFKCY | 67 |
| | | GSGGSG-RBD(131)- | GVSATKLNDLCFSNVYADSFVVKGDDVRQIAPGQTGVIADYNYKLPDDFMGCVLAWNTRNIDATSTG | |
| | | Ferritin | NYNYKRRYLRHGKLRPFERDISNVPFSPDGKPCTPPALNCYWPLNDYGFYTTTGIGYQPYRVVVLSFE | |
| | | | NKSAPATVCGPGSGGSGNITNLCPFGEVFNATRFASVYAWNRKRISNCVADYSVLYNSASFSTFKCY | |
| | | | GVSPTKLNDLCFTNVYADSFVIRGDEVRQIAPGQTGKIADYNYKLPDDFTGCVIAWNSNNLDSKVGGN | |
| | | | YNYLRRLFRKSNLKPFERDISTEIYQAGSTPCNGVEGFNCYFPLQSYGFQPTNGVGYQPYRVVVLSFE | |
| | | | NKSAPATVCGPGSGGGGESQVRQQFSKDIEKLLNEQVNKEMQSSNLYMSMSSWCYTHSLDGAGLF | |
| | | | LFDHAAEEYEHAKKLIIFLNENNVPVQLTSISAPEHKFEGLTQIFQKAYEHEQHISESINNIVDHAIKSKDH | |
| | | | ATFNFLQWYVAEQHEEEVLFKDILDKIELIGNENHGLYLADQYVKGIAKSRKSGS | |
| pCoV302 | RBD-S1-Ferritin | His8-3c-R-S1-FN where SARS- | HHHHHHHHGPLEVLFQGPNITNLCPFGEVFNATKFPSVYAWERKKISNCVADYSVLYNSTFFSTFKCY | 68 |
| | | 1 RBD(131like) is added | GVSATKLNDLCFSNVYADSFVVKGDDVRQIAPGQTGVIADYNYKLPDDFMGCVLAWNTRNIDATSTG | |
| | | | NYNYKRRYLRHGKLRPFERDISNVPFSPDGKPCTPPALNCYWPLNDYGFYTTTGIGYQPYRVVVLSFE | |
| | | | NKSAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoV303 | RBD-RBD-NTD- | His8-3c-RRN-FN where SARS-1 | HHHHHHHHGPLEVLFQGPNITNLCPFGEVFNATKFPSVYAWERKKISNCVADYSVLYNSTFFSTFKCY | 69 |
| | Ferritin | RBD(131like) is added to | GVSATKLNDLCFSNVYADSFVVKGDDVRQIAPGQTGVIADYNYKLPDDFMGCVLAWNTRNIDATSTG | |
| | | pCoV146 | NYNYKRRYLRHGKLRPFERDISNVPFSPDGKPCTPPALNCYWPLNDYGFYTTTGIGYQPYRVVVLSFE | |
| | | | NKSAPATVCGPGSGGNITNLCPFGEVFNATRFASVYAWNRKRISNCVADYSVLYNSASFSTFKCYGV | |
| | | | SPTKLNDLCFTNVYADSFVIRGDEVRQIAPGQTGKIADYNYKLPDDFTGCVIAWNSNNLDSKVGGNYN | |
| | | | YLRRLFRKSNLKPFERDISTEIYQAGSTPCNGVEGFNCYFPLQSYGFQPTNGVGYQPYRVVVLSFENK | |
| | | | SAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLGSGGGGESQVRQQFSKDIEKLLNEQVNKEMQSSN | |
| | | | LYMSMSSWCYTHSLDGAGLFLFDHAAEEYEHAKKLIIFLNENNVPVQLTSISAPEHKFEGLTQIFQKAY | |
| | | | EHEQHISESINNIVDHAIKSKDHATFNFLQWYVAEQHEEEVLFKDILDKIELIGNENHGLYLADQYVKGIA | |
| | | | KSRKSGS | |
| pCoV304 | RBD-RBD-NTD- | His8-3c-RNRN-FN where SARS- | HHHHHHHHGPLEVLFQGPNITNLCPFGEVFNATKFPSVYAWERKKISNCVADYSVLYNSTFFSTFKCY | 70 |
| | Ferritin | 1 RBD(131like) and NTD | GVSATKLNDLCFSNVYADSFVVKGDDVRQIAPGQTGVIADYNYKLPDDFMGCVLAWNTRNIDATSTG | |
| | | are added to pCoV146 | NYNYKRRYLRHGKLRPFERDISNVPFSPDGKPCTPPALNCYWPLNDYGFYTTTGIGYQPYRVVVLSFE | |
| | | | NKSAPATVCGPGSGGLDRCTTFDDVQAPNYTQHTSSMRGVYYPDEIFRSDTLYLTQDLFLPFYSNVT | |
| | | | GFHTINHTFGNPVIPFKDGIYFAATEKSNVVRGWVFGSTMNNKSQSVIIINNSTNWVIRACNFELCDNPF | |
| | | | FAVSKPMGTQTHTMIFDNAFNCTFEYISDAFSLDVSEKSGNFKHLREFVFKNKDGFLYVYKGYQPIDWV | |
| | | | RDLPSGFNTLKPIFKLPLGINITNFRAILTAFSPAQDIWGTSAAAYFVGYLKPTTFMLKYDENGTITDAVD | |
| | | | CSQNPLAELKCSVGSGGSGNITNLCPFGEVFNATRFASVYAWNRKRISNCVADYSVLYNSASFSTFKC | |
| | | | YGVSPTKLNDLCFTNVYADSFVIRGDEVRQIAPGQTGKIADYNYKLPDDFTGCVIAWNSNNLDSKVGG | |
| | | | NYNYLRRLFRKSNLKPFERDISTEIYQAGSTPCNGVEGFNCYFPLQSYGFQPTNGVGYQPYRVVVLSF | |
| | | | ENKSAPATVCGPGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLGSGGGGESQVRQQFSKDIEKLLNEQVNKEMQ | |
| | | | SSNLYMSMSSWCYTHSLDGAGLFLFDHAAEEYEHAKKLIIFLNENNVPVQLTSISAPEHKFEGLTQIFQ | |
| | | | KAYEHEQHISESINNIVDHAIKSKDHATFNFLQWYVAEQHEEEVLFKDILDKIELIGNENHGLYLADQYV | |
| | | | KGIAKSRKSGS | |
| pCoV305 | RBD inserted into | His8-3c-R2N-FN-70 where | HHHHHHHHGPLEVLFQGPNITNLCPFGEVFNATRFASVYAWNRKRISNCVADYSVLYNSASFSTFKCY | 71 |
| | NTD | SARS-1 RBD(131like) is | GVSPTKLNDLCFTNVYADSFVIRGDEVRQIAPGQTGKIADYNYKLPDDFTGCVIAWNSNNLDSKVGGN | |
| | loop-Ferritin | inserted into the NTD | YNYLRRLFRKSNLKPFERDISTEIYQAGSTPCNGVEGFNCYFPLQSYGFQPTNGVGYQPYRVVVLSFE | |
| | | (loop at 70) in pCoV146 | NKSAPATVCGPGSGGSGSSQCVNLTTRTQLPPAYTNSFTRGVYYPDKVFRSSVLHSTQDLFLPFFSN | |
| | | | VTWFHAIHVGSGGSGNITNLCPFGEVFNATKFPSVYAWERKKISNCVADYSVLYNSTFFSTFKCYGVS | |
| | | | ATKLNDLCFSNVYADSFVVKGDDVRQIAPGQTGVIADYNYKLPDDFMGCVLAWNTRNIDATSTGNYN | |
| | | | YKRRYLRHGKLRPFERDISNVPFSPDGKPCTPPALNCYWPLNDYGFYTTTGIGYQPYRVWLSFENKS | |
| | | | APATVCGPGSGGSGSGTNGTKRFDNPVLPFNDGVYFASTEKSNIIRGWIFGTTLDSKTQSLLIVNNAT | |
| | | | NVVIKVCEFQFCNDPFLGVYYHKNNKSWMESEFRVYSSANNCTFEYVSQPFLMDLEGKQGNFKNLR | |
| | | | EFVFKNIDGYFKIYSKHTPINLVRDLPQGFSALEPLVDLPIGINITRFQTLLALHRSYLTPGDSSSGWTAG | |
| | | | AAAYYVGYLQPRTFLLKYNENGTITDAVDCALDPLSETKCTLGSGGGGESQVRQQFSKDIEKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoV306 | RBD inserted into | His8-3c-R2N-FN-148 where | HHHHHHHHGPLEVLFQGPNITNLCPFGEVFNATRFASVYAWNRKRISNCVADYSVLYNSASFSTFKCY | 72 |
| | NTD | SARS-1 RBD(131like) is | GVSPTKLNDLCFTNVYADSFVIRGDEVRQIAPGQTGKIADYNYKLPDDFTGCVIAWNSNNLDSKVGGN | |
| | loop-Ferritin | inserted into the NTD | YNYLRRLFRKSNLKPFERDISTEIYQAGSTPCNGVEGFNCYFPLQSYGFQPTNGVGYQPYRVVVLSFE | |
| | | (loop at 148) in pCoV146 | NKSAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNGSGGSGNITNLCPFGEVFNATKFPSVYAWERKKISNCVADYSVLYNSTFFS | |
| | | | TFKCYGVSATKLNDLCFSNVYADSFVVKGDDVRQIAPGQTGVIADYNYKLPDDFMGCVLAWNTRNIDA | |
| | | | TSTGNYNYKRRYLRHGKLRPFERDISNVPFSPDGKPCTPPALNCYWPLNDYGFYTTTGIGYQPYRVV | |
| | | | VLSFENKSAPATVCGPGSGGSGNKSWMESEFRVYSSANNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLGSGGGGESQVRQQFSKDIEKLLNEQVNK | |
| | | | EMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoV307 | RBD inserted into | His8-3c-R2N-FN-166 where | HHHHHHHHGPLEVLFQGPNITNLCPFGEVFNATRFASVYAWNRKRISNCVADYSVLYNSASFSTFKCY | 73 |
| | NTD | SARS-1 RBD(131like) is | GVSPTKLNDLCFTNVYADSFVIRGDEVRQIAPGQTGKIADYNYKLPDDFTGCVIAWNSNNLDSKVGGN | |
| | loop-Ferritin | inserted into the NTD | YNYLRRLFRKSNLKPFERDISTEIYQAGSTPCNGVEGFNCYFPLQSYGFQPTNGVGYQPYRVVVLSFE | |
| | | (loop at 166) in pCoV146 | NKSAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANGSGGSGNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKRRYLRHGKLRPFERDISNVPFSPDGKPCTPPALNCYWPLND | |
| | | | YGFYTTTGIGYQPYRVVVLSFENKSAPATVCGPGSGGSGNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLGSGGGGESQVRQQFSKDIEKLLNEQVNK | |
| | | | EMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoV311 | R-mosaic SpFN | His8-3c-R-MERS-SpFN where | HHHHHHHHGPLEVLFQGPNLPDCNIEEWLTAKSVPSPLNWERKTFSNCNFNLSSLLRFVQADSFSCN | 74 |
| | | Clu2 RBD is added | NIDASKVYGMCFGSISVDKFAIPNSRQVDLQLGNSGFLQSFNYKIDTTATSCQLYYSLPANNVTINNYN | |
| | | | PSSWNRRYGFNDDTVFKPRPAGVLTNHDVVYSQQCFKVPNTFCPCKNNGSCVSSGKGTTNGSGTC | |
| | | | PAGTNYRTCENTTWCACSCTPDPITASGPRKCPQKKSLVGVGEHCSGLGVDSDYCGNVSCLCSTDA | |
| | | | FLGWSADSCLVNDRCNIFANIILNGINSGTTCSTGSGGSGYVDVGPDSVKSACIEVDIQQTFFDKTWP | |
| | | | RPIDVSKADGIIYPQGRTYSNITITYQGLFPYQGDHGDMYVYSAGHATGTTPQKLFVANYSQDVKQFA | |
| | | | NGFVVRIGAAANSTGTVIISPSTSATIRKIYPAFMLGSSVGNFSDGKMGRFFNHTLVLLPDGCGTLLRAF | |
| | | | YCILEPRSGNHCPAGNSYTSFATYHTPATDCSDGNYNRNASLNSFKEYFNLRNCTFMYTYNITEDEILE | |
| | | | WFGITQTAQGVHLFSSRYVDLYGGNMFQFATLPVYDTIKYYSIIPHSIRSIQSDRKAWAAFYVYKLQPL | |
| | | | TFLLDFSVDGYIRRAIDCGFNDLSQLHCSYESFDVESGVYSVSSFEAKPSGSVVEQAEGVECDFSPLL | |
| | | | SGTPPQVYNFKRLVFTNCNYNLTKLLSLFSVNDFTCSQISPAAIASNCYSSLILDYFSYPLSMKSDLSVS | |
| | | | SAGPISQFNYKQSFSNPTCLILATVPHNLTTITKPLKYSYINKCSRFLSDDRTEVPQLVNANQYSPCVSIV | |
| | | | PSTVWEDGDYYRKQLSPLEGGGWLVASGSTVAMTEQLQMGFGITVQYGTDTNSVCPKLEFANDTKI | |
| | | | ASQLGNCVEYSLYGVSGRGVFQNCTAVGVRQQRFVYDAYQNLVGYYSDDGNYYCLRACVSVPVSVI | |
| | | | YDKETKTHATLFGSVACEHISSTMSQYSRSTRSMLKRRDSTYGPLQTPVGCVLGLVNSSLFVEDCKLP | |
| | | | LGQSLCALPDTPSTLTPASVGSVPGEMRLASIAFNHPIQVDQLNSSYFKLSIPTNFSFGVTQEYIQTTIQ | |
| | | | KVTVDCKQYVCNGFQKCEQLLREYGQFCSKINQALHGANLRQDDSVRNLFASVKSSQSSPIIPGFGG | |
| | | | DFNLTLLEPVSISTGSRSARSAIEDLLFDKVTIADPGYMQGYDDCMQQGPASARDLICAQYVAGYKVLP | |
| | | | PLMDVNMEAAYTSSLLGSIAGVGWTAGLSSFAAIPFAQSIFYRLNGVGITQQVLSENQKLIANKFNQAL | |
| | | | GAMQTGFTTTNEAFHKVQDAVNNNAQALSKLASELSNTFGAISASIGDIIQRLDPPEQDAQIDRLINGRL | |
| | | | TTLNAFVAQQLVRSESAALSAQLAKDKVNECVKAQSKRSGFCGQGTHIVSFVVNAPNGLYFMHVGYY | |
| | | | PSNHIEVVSAYGLCDAANPTNCIAPVNGYFIKTNNTRIVDEWSYTGSSFYAPEPITSLNTKYVAPQVTY | |
| | | | QNISTNLPPPLLGNSTGIDFQDELDEIHKQGSGGSGDIIKLLNEQVNKEMQSSNLYMSMSSWCYTHSL | |
| | | | DGAGLFLFDHAAEEYEHAKKLIIFLNENNVPVQLTSISAPEHKFEGLTQIFQKAYEHEQHISESINNIVDH | |
| | | | AIKSKDHATFNFLQWYVAEQHEEEVLFKDILDKIELIGNENHGLYLADQYVKGIAKSRKSGS | |
| pCoV312 | R-mosaic SpFN | His8-3c-R-SpFN where Clu2 | HHHHHHHHGPLEVLFQGPNLPDCNIEEWLTAKSVPSPLNWERKTFSNCNFNLSSLLRFVQADSFSCN | 75 |
| | | RBD is added | NIDASKVYGMCFGSISVDKFAIPNSRQVDLQLGNSGFLQSFNYKIDTTATSCQLYYSLPANNVTINNYN | |
| | | | PSSWNRRYGFNDDTVFKPRPAGVLTNHDVVYSQQCFKVPNTFCPCKNNGSCVSSGKGTTNGSGTC | |
| | | | PAGTNYRTCENTTWCACSCTPDPITASGPRKCPQKKSLVGVGEHCSGLGVDSDYCGNVSCLCSTDA | |
| | | | FLGWSADSCLVNDRCNIFANIILNGINSGTTCSTGSGGSGSSQCVNLTTRTQLPPAYTNSFTRGVYYP | |
| | | | DKVFRSSVLHSTQDLFLPFFSNVTWFHAIHVSGTNGTKRFDNPVLPFNDGVYFASTEKSNIIRGWIFGT | |
| | | | TLDSKTQSLLIVNNATNVVIKVCEFQFCNDPFLGVYYHKNNKSWMESEFRVYSSANNCTFEYVSQPFL | |
| | | | MDLEGKQGNFKNLREFVFKNIDGYFKIYSKHTPINLVRDLPQGFSALEPLVDLPIGINITRFQTLLALHRS | |
| | | | YLTPGDSSSGWTAGAAAYYVGYLQPRTFLLKYNENGTITDAVDCALDPLSETKCTLKSFTVEKGIYQT | |
| | | | SNFRVQPTESIVRFPNITNLCPFGEVFNATRFASVYAWNRKRISNCVADYSVLYNSASFSTFKCYGVSP | |
| | | | TKLNDLCFTNVYADSFVIRGDEVRQIAPGQTGKIADYNYKLPDDFTGCVIAWNSNNLDSKVGGNYNYL | |
| | | | YRLFRKSNLKPFERDISTEIYQAGSTPCNGVEGFNCYFPLQSYGFQPTNGVGYQPYRVWVLSFELLHA | |
| | | | PATVCGPKKSTNLVKNKCVNFNFNGLTGTGVLTESNKKFLPFQQFGRDIADTTDAVRDPQTLEILDITP | |
| | | | CSFGGVSVITPGTNTSNQVAVLYQDVNCTEVPVAIHADQLTPTWRVYSTGSNVFQTRAGCLIGAEHVN | |
| | | | NSYECDIPIGAGICASYQTQTNSPGSASSVASQSIIAYTMSLGAENSVAYSNNSIAIPTNFTISVTTEILPV | |
| | | | SMTKTSVDCTMYICGDSTECSNLLLQYGSFCTQLNRALTGIAVEQDKNTQEVFAQVKQIYKTPPIKDFG | |
| | | | GFNFSQILPDPSKPSKRSFIEDLLFNKVTLADAGFIKQYGDCLGDIAARDLICAQKFNGLTVLPPLLTDE | |
| | | | MIAQYTSALLAGTITSGWTFGAGAALQIPFAMQMAYRFNGIGVTQNVLYENQKLIANQFNSAIGKIQDS | |
| | | | LSSTASALGKLQDVVNQNAQALNTLVKQLSSNFGAISSVLNDILSRLDPPEAEVQIDRLITGRLQSLQTY | |
| | | | VTQQLIRAAEIRASANLAATKMSECVLGQSKRVDFCGKGYHLMSFPQSAPHGVVFLHVTYVPAQEKN | |
| | | | FTTAPAICHDGKAHFPREGVFVSNGTHWFVTQRNFYEPQIITTDNTFVSGNCDVVIGIVNNTVYDPLQS | |
| | | | ELDSIKEELDKIHKNGSGGSGDIIKLLNEQVNKEMQSSNLYMSMSSWCYTHSLDGAGLFLFDHAAEEY | |
| | | | EHAKKLIIFLNENNVPVQLTSISAPEHKFEGLTQIFQKAYEHEQHISESINNIVDHAIKSKDHATFNFLQW | |
| | | | YVAEQHEEEVLFKDILDKIELIGNENHGLYLADQYVKGIAKSRKSGS | |
| pCoV313 | R-mosaic SpFN | His8-3c-R-SpFN where the | HHHHHHHHGPLEVLFQGPNLPDCNIEEWLTAKSVPSPLNWERKTFSNCNFNLSSLLRFVQADSFSCN | 76 |
| | | RBD is replaced with Clu3 | NIDASKVYGMCFGSISVDKFAIPNSRQVDLQLGNSGFLQSFNYKIDTTATSCQLYYSLPANNVTINNYN | |
| | | RBD and Clu2 RBD is added | PSSWNRRYGFNDDTVFKPRPAGVLTNHDVVYSQQCFKVPNTFCPCKNNGSCVSSGKGTTNGSGTC | |
| | | to N-term | PAGTNYRTCENTTWCACSCTPDPITASGPRKCPQKKSLVGVGEHCSGLGVDSDYCGNVSCLCSTDA | |
| | | | FLGWSADSCLVNDRCNIFANIILNGINSGTTCSTGSGGSGSSQCVNLTTRTQLPPAYTNSFTRGVYYP | |
| | | | DKVFRSSVLHSTQDLFLPFFSNVTWFHAIHVSGTNGTKRFDNPVLPFNDGVYFASTEKSNIIRGWIFGT | |
| | | | TLDSKTQSLLIVNNATNVVIKVCEFQFCNDPFLGVYYHKNNKSWMESEFRVYSSANNCTFEYVSQPFL | |
| | | | MDLEGKQGNFKNLREFVFKNIDGYFKIYSKHTPINLVRDLPQGFSALEPLVDLPIGINITRFQTLLALHRS | |
| | | | YLTPGDSSSGWTAGAAAYYVGYLQPRTFLLKYNENGTITDAVDCALDPLSETKCTLKSFTVEKGIYQT | |
| | | | SNFRVQPTESIVRFPQGVECDFSKLFKAAAPQIYNFSRLVFTNCNYNLTKLLSLFQVDEFSCHQISPSAL | |
| | | | ASGCYSSLTVDYFAYPLSMKSYLQIGSAGEISQFNYKQDFSNPTCRILASVPANVSITKPDKYIYISQCY | |
| | | | SLSAYSDVDVPHYVNPGQYTSIPCLYLTSSGFDEDGQYFRYNLTQFEGQNYLVATGVTVSMTDNLQM | |
| | | | VFVITVQYGTDTNSVCPKKSTNLVKNKCVNFNFNGLTGTGVLTESNKKFLPFQQFGRDIADTTDAVRD | |
| | | | PQTLEILDITPCSFGGVSVITPGTNTSNQVAVLYQDVNCTEVPVAIHADQLTPTWRVYSTGSNVFQTRA | |
| | | | GCLIGAEHVNNSYECDIPIGAGICASYQTQTNSPGSASSVASQSIIAYTMSLGAENSVAYSNNSIAIPTN | |
| | | | FTISVTTEILPVSMTKTSVDCTMYICGDSTECSNLLLQYGSFCTQLNRALTGIAVEQDKNTQEVFAQVK | |
| | | | QIYKTPPIKDFGGFNFSQILPDPSKPSKRSFIEDLLFNKVTLADAGFIKQYGDCLGDIAARDLICAQKFNG | |
| | | | LTVLPPLLTDEMIAQYTSALLAGTITSGWTFGAGAALQIPFAMQMAYRFNGIGVTQNVLYENQKLIANQ | |
| | | | FNSAIGKIQDSLSSTASALGKLQDVVNQNAQALNTLVKQLSSNFGAISSVLNDILSRLDPPEAEVQIDRLI | |
| | | | TGRLQSLQTYVTQQLIRAAEIRASANLAATKMSECVLGQSKRVDFCGKGYHLMSFPQSAPHGVVFLH | |
| | | | VTYVPAQEKNFTTAPAICHDGKAHFPREGVFVSNGTHWFVTQRNFYEPQIITTDNTFVSGNCDVVIGIV | |
| | | | NNTVYDPLQSELDSIKEELDKIHKNGSGGSGDIIKLLNEQVNKEMQSSNLYMSMSSWCYTHSLDGAGL | |
| | | | FLFDHAAEEYEHAKKLIIFLNENNVPVQLTSISAPEHKFEGLTQIFQKAYEHEQHISESINNIVDHAIKSKD | |
| | | | HATFNFLQWYVAEQHEEEVLFKDILDKIELIGNENHGLYLADQYVKGIAKSRKSGS | |
| pCoV316 | R-SpFN | R-SpFN with SpFN-HexaPro | HHHHHHHHGPLEVLFQGPNITNLCPFGEVFNATKFPSVYAWERKKISNCVADYSVLYNSTFFSTFKCY | 77 |
| | | with SARS-1 RBD(131like) | GVSATKLNDLCFSNVYADSFVVKGDDVRQIAPGQTGVIADYNYKLPDDFMGCVLAWNTRNIDATSTG | |
| | | added | NYNYKRRYLRHGKLRPFERDISNVPFSPDGKPCTPPALNCYWPLNDYGFYTTTGIGYQPYRVVVLSFE | |
| | | | NKSAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTQTN | |
| | | | SPGSASSVASQSIIAYTMSLGAENSVAYSNNSIAIPTNFTISVTTEILPVSMTKTSVDCTMYICGDSTECS | |
| | | | NLLLQYGSFCTQLNRALTGIAVEQDKNTQEVFAQVKQIYKTPPIKDFGGFNFSQILPDPSKPSKRSPIED | |
| | | | LLFNKVTLADAGFIKQYGDCLGDIAARDLICAQKFNGLTVLPPLLTDEMIAQYTSALLAGTITSGWTFGA | |
| | | | GPALQIPFPMQMAYRFNGIGVTQNVLYENQKLIANQFNSAIGKIQDSLSSTPSALGKLQDVVNQNAQAL | |
| | | | NTLVKQLSSNFGAISSVLNDILSRLDPPEAEVQIDRLITGRLQSLQTYVTQQLIRAAEIRASANLAATKMS | |
| | | | ECVLGQSKRVDFCGKGYHLMSFPQSAPHGVVFLHVTYVPAQEKNFTTAPAICHDGKAHFPREGVFVS | |
| | | | NGTHWFVTQRNFYEPQIITTDNTFVSGNCDWVIGIVNNTVYDPLQSELDSIKEELDKIHKNGSGGSGDII | |
| | | | KLLNEQVNKEMQSSNLYMSMSSWCYTHSLDGAGLFLFDHAAEEYEHAKKLIIFLNENNVPVQLTSISAP | |
| | | | EHKFEGLTQIFQKAYEHEQHISESINNIVDHAIKSKDHATFNFLQWYVAEQHEEEVLFKDILDKIELIGNE | |
| | | | NHGLYLADQYVKGIAKSRKSGS | |
| pCoV317 | R-SpFN | R-SpFN with original SpFN | HHHHHHHHGPLEVLFQGPNITNLCPFGEVFNATKFPSVYAWERKKISNCVADYSVLYNSTFFSTFKCY | 78 |
| | | with SARS-1 RBD(131like) | GVSATKLNDLCFSNVYADSFVVKGDDVRQIAPGQTGVIADYNYKLPDDFMGCVLAWNTRNIDATSTG | |
| | | added | NYNYKRRYLRHGKLRPFERDISNVPFSPDGKPCTPPALNCYWPLNDYGFYTTTGIGYQPYRVVVLSFE | |
| | | | NKSAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTQTN | |
| | | | SPGSASSVASQSIIAYTMSLGAENSVAYSNNSIAIPTNFTISVTTEILPVSMTKTSVDCTMYICGDSTECS | |
| | | | NLLLQYGSFCTQLNRALTGIAVEQDKNTQEVFAQVKQIYKTPPIKDFGGFNFSQILPDPSKPSKRSFIED | |
| | | | LLFNKVTLADAGFIKQYGDCLGDIAARDLICAQKFNGLTVLPPLLTDEMIAQYTSALLAGTITSGWTFGA | |
| | | | GAALQIPFAMQMAYRFNGIGVTQNVLYENQKLIANQFNSAIGKIQDSLSSTASALGKLQDVVNQNAQAL | |
| | | | NTLVKQLSSNFGAISSVLNDILSRLDPPEAEVQIDRLITGRLQSLQTYVTQQLIRAAEIRASANLAATKMS | |
| | | | ECVLGQSKRVDFCGKGYHLMSFPQSAPHGVVFLHVTYVPAQEKNFTTAPAICHDGKAHFPREGVFVS | |
| | | | NGTHWFVTQRNFYEPQIITTDNTFVSGNCDVVIGIVNNTVYDPLQSELDSIKEELDKIHKNGSGGSGDII | |
| | | | KLLNEQVNKEMQSSNLYMSMSSWCYTHSLDGAGLFLFDHAAEEYEHAKKLIIFLNENNVPVQLTSISAP | |
| | | | EHKFEGLTQIFQKAYEHEQHISESINNIVDHAIKSKDHATFNFLQWYVAEQHEEEVLFKDILDKIELIGNE | |
| | | | NHGLYLADQYVKGIAKSRKSGS | |
| pCoV247 | RBD-RBD-Ferritin | His8-3c-RBD(131)-GSGGSG- | HHHHHHHHGPLEVLFQGPNITNLCPFGEVFNATRFASVYAWNRKRISNCVADYSVLYNSASFSTFKCY | 79 |
| | | M3RBD-Ferritin | GVSPTKLNDLCFTNVYADSFVIRGDEVRQIAPGQTGKIADYNYKLPDDFTGCVIAWNSNNLDSKVGGN | |
| | | | YNYLRRLFRKSNLKPFERDISTEIYQAGSTPCNGVEGFNCYFPLQSYGFQPTNGVGYQPYRVVVLSFE | |
| | | | NKSAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVWEDGDYYRKQLSPLEGGGWLVASGSTVAMTEQL | |
| | | | QMGFGITVQNGTDTNSVCPKGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoV248 | RBD-RBD-Ferritin | His8-3c-RBD(127)-GSGGSG- | HHHHHHHHGPLEVLFQGPNITNLCPFGEVFNATRFASVYAWNRKRISNCVADYSVLYNSASFSTFKCY | 80 |
| | | M3RBD-Ferritin | GVSPTKLNDLCFTNVYADSFVIRGDEVRQIAPGQTGKIADYNYKLPDDFTGCVIAWNSNNLDSKVGGN | |
| | | | YNYLYRLFRKSNLKPFERDISTEIYQAGSTPCNGVEGFNCYAPLQSYGFQPTNGVGYQPYRVVVLSFE | |
| | | | NKSAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVWEDGDYYRKQLSPLEGGGWLVASGSTVAMTEQL | |
| | | | QMGFGITVQNGTDTNSVCPKGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoV249 | RBD-RBD-Ferritin | His8-3c-M3RBD-GSGGSG- | HHHHHHHHGPLEVLFQGPEGVECDFSPLLSGTPPQVYNFKRLVFTNCNYNLTKLLSLFSVNDFTCSQI | 81 |
| | | RBD(131)-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGSGNITNLCPFGEVFNATRFASVYAWNRKRISNCVADYSVLYN | |
| | | | SASFSTFKCYGVSPTKLNDLCFTNVYADSFVIRGDEVRQIAPGQTGKIADYNYKLPDDFTGCVIAWNS | |
| | | | NNLDSKVGGNYNYLRRLFRKSNLKPFERDISTEIYQAGSTPCNGVEGFNCYFPLQSYGFQPTNGVGY | |
| | | | QPYRVVVLSFENKSAPATVCGPGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pCoV250 | RBD-RBD-Ferritin | His8-3c-M3RBD-GSGGSG- | HHHHHHHHGPLEVLFQGPEGVECDFSPLLSGTPPQVYNFKRLVFTNCNYNLTKLLSLFSVNDFTCSQI | 82 |
| | | RBD(127)-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGSGNITNLCPFGEVFNATRFASVYAWNRKRISNCVADYSVLYN | |
| | | | SASFSTFKCYGVSPTKLNDLCFTNVYADSFVIRGDEVRQIAPGQTGKIADYNYKLPDDFTGCVIAWNS | |
| | | | NNLDSKVGGNYNYLYRLFRKSNLKPFERDISTEIYQAGSTPCNGVEGFNCYAPLQSYGFQPTNGVGY | |
| | | | QPYRVVVLSFENKSAPATVCGPGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pMR01 | RBD insert | RBD-His6 from | NITQLCPFNEVFNITSFPSVYAWERMRITNCVADYSVLYNSSASFSTFQCYGVSPTKLNDLCFSSVYAD | 83 |
| | | NC_014470.BM4831.BGR.2008 | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLASYGFTQSSGIGFQPYRVVVLSFELLNAPATVCGPGSHHHHHH | |
| pMR01W | RBD insert | MR01 RBD-His6-T498W | NITQLCPFNEVFNITSFPSVYAWERMRITNCVADYSVLYNSSASFSTFQCYGVSPTKLNDLCFSSVYAD | 84 |
| | | | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLASYGFWQSSGIGFQPYRVVVLSFELLNAPATVCGPGSHHHHHH | |
| pMR01YW | RBD insert | MR01 RBD-His6-A493Y-T498W | NITQLCPFNEVFNITSFPSVYAWERMRITNCVADYSVLYNSSASFSTFQCYGVSPTKLNDLCFSSVYAD | 85 |
| | | | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLYSYGFWQSSGIGFQPYRVVVLSFELLNAPATVCGPGSHHHHHH | |
| pMR02 | RBD insert | RBD-His6 from | NITNLCPFGEVENATTFPSVYAWERKRISNCVADYSVLYNSTSFSTFKCYGVSATKLNDLCFSNVYADS | 86 |
| | | KC881005.RsSHC014 | FVVKGDDVRQIAPGQTGVIADYNYKLPDDFLGCVLAWNTNSKDSSTSGNYNYLYRWVRRSKLNPYER | |
| | | | DLSNDIYSPGGQSCSAVGPNCYNPLRPYGFFTTAGVGHQPYRVVVLSFELLNAPATVCGPGSHHHHH | |
| | | | H | |
| pMR03 | RBD insert | RBD-His6 from | NITNLCPFGEVFNATTFPSVYAWERKRISNCVADYSVLYNSTSFSTFKCYGVSATKLNDLCFSNVYADS | 87 |
| | | KC881007.Bat.WIV1 | FVVKGDDVRQIAPGQTGVIADYNYKLPDDFTGCVLAWNTRNIDATQTGNYNYKYRSLRHGKLRPFER | |
| | | | DISNVPFSPDGKPCTPPAFNCYWPLNDYGFYITNGIGYQPYRVVVLSFELLNAPATVCGPGSHHHHHH | |
| pMR04 | RBD insert | RBD-His6 from AVP78042_ | NITNVCPFHKVFNATRFPSVYAWERTKISDCIADYTVFYNSTSFSTFKCYGVSPSKLIDLCFTSVYADTF | 88 |
| | | BatSARSlikecoronavirus | LIRFSEVRQVAPGQTGVIADYNYKLPDDFTGCVIAWNTAKQDTGHYFYRSHRSTKLKPFERDLSSDEN | |
| | | | GVRTLSTYDFNPNVPLEYQATRVVVLSFELLNAPATVCGPGSHHHHHH | |
| pMR05 | RBD insert | RBD-His6 from | NITNLCPFGEVFNATTFASVYAWNRKRISNCVADYSVLYNSTSFSTFKCYGVSPTKLNDLCFTNVYADS | 89 |
| | | MZ937001_BANAL20103_ | FVVRGDEVRQIAPGQTGKIADYNYKLPDDFTGCVIAWNSNNLDSKVGGNYNYLYRLFRKSNLKPFERD | |
| | | Laos_2020 | ISTEIYQAGSTPCNGVEGFNCYFPLQSYGFHPTNGVGYQPYRVVVLSFELLNAPATVCGPGSHHHHH | |
| | | | IH | |
| pMR06 | RBD insert | RBD-His6 from | NITNLCPFGEVFNATTFASVYAWNRKRISNCVADYSVLYNSTSFSTFKCYGVSPTKLNDLCFTNVYADS | 90 |
| | | MN996532.2013.Bat.RaTG13 | FVITGDEVRQIAPGQTGKIADYNYKLPDDFTGCVIAWNSKHIDAKEGGNFNYLYRLFRKANLKPFERDI | |
| | | | STEIYQAGSKPCNGQTGLNCYYPLYRYGFYPTDGVGHQPYRVWVLSFELLNAPATVCGPGSHHHHHH | |
| pMR08 | RBD insert | RBD-His6 from MZ937004_ | NITNFCPFDKVFNATRFPNVYAWQRTKISDCIADYTVLYNSTSFSTFKCYGVSPSKLIDLCFTSVYADTF | 91 |
| | | BANAL20247_Laos_2020 | LIRFSEVRQIAPGETGVIADYNYKLPDDFTGCVLAWNTAQQDIGSYFYRSHRAVKLKPFERDLSSDEN | |
| | | | GVRTLSTYDFNPNVPLDYQATRVVVLSFELLNAPATVCGPGSHHHHHH | |
| pMR08W | RBD insert | MR08 RBD-His6-N498W | NITNFCPFDKVFNATRFPNVYAWQRTKISDCIADYTVLYNSTSFSTFKCYGVSPSKLIDLCFTSVYADTF | 92 |
| | | | LIRFSEVRQIAPGETGVIADYNYKLPDDFTGCVLAWNTAQQDIGSYFYRSHRAVKLKPFERDLSSDEN | |
| | | | GVRTLSTYDFWPNVPLDYQATRVVVLSFELLNAPATVCGPGSHHH | |
| pMR08YW | RBD insert | MR08 RBD-His6-S493Y-N498W | NITNFCPFDKVFNATRFPNVYAWQRTKISDCIADYTVLYNSTSFSTFKCYGVSPSKLIDLCFTSVYADTF | 93 |
| | | | LIRFSEVRQIAPGETGVIADYNYKLPDDFTGCVLAWNTAQQDIGSYFYRSHRAVKLKPFERDLSSDEN | |
| | | | GVRTLYTYDFWPNVPLDYQATRVVVLSFELLNAPATVCGPGSHHHHHH | |
| pMR09 | RBD insert | RBD-His6 from ATO98145_ | NITNLCPFDAVFNATRFPSVYAWERVKISNCVADYTAFYNSTSFSTFKCYGVSPSKLIDLCFTSVYADT | 94 |
| | | BatSARSlikecoronavirus | FLIRFSEVRQVAPGETGVIADYNYKLPDDFTGCVIAWNTAKQDVGSYFYRSHRSSKLKPFERDLSSDE | |
| | | | NGVRTLSTYDFNPNVPLDYQATRVVVLSFELLNAPATVCGPGSHHHHHH | |
| pMR10 | RBD insert | RBD-His6 from JX993987. | NITNRCPFDKVFNATRFPSVYAWERTKISDCVADYTVLYNSTSFSTFKCYGVSPSKLIDLCFTSVYADT | 95 |
| | | 2011.BatRp.Shaanxi2011 | FLIRSSEVRQVAPGETGVIADYNYKLPDDFTGCVIAWNTANQDQGQYYYRSSRKEKLKPFERDLSSDE | |
| | | | NGVYTLSTYDFYPSVPLDYQATRVVVLSFELLNAPATVCGPGSHHHHHH | |
| pMR11 | RBD insert | RBD-His6 from KJ473812. | NITNLCPFDKVFNATRFPSVYAWERTKISDCVADYTVFYNSTSFSTFNCYGVSPSKLIDLCFTSVYADT | 96 |
| | | BtRfBetaCoV.HeB2013 | FLIRFSEVRQVAPGQTGVIADYNYKLPDDFTGCVIAWNTAKQDVGSYFYRSHRSSKLKPFERDLSSEE | |
| | | | NGVRTLSTYDFNQYVPLEYQATRVVVLSFELLNAPATVCGPGSHHHHHH | |
| pMR12 | RBD insert | RBD-His6 from | NITNRCPFDKVFNATRFPNVYAWERTKISDCVADYTVLYNSTSFSTFKCYGVSPSKLIDLCFTSVYADT | 97 |
| | | DQ071615.2003.Bat.Rp3 | FLIRSSEVRQVAPGETGVIADYNYKLPDDFTGCVIAWNTAKQDQGQYYYRSHRKTKLKPFERDLSSDE | |
| | | | NGVRTLSTYDFYPSVPVAYQATRVVVLSFELLNAPATVCGPGSHHHHHH | |
| pMR13 | RBD insert | RBD-His6 from | NITNRCPFDKVFNASRFPNVYAWERTKISDCVADYTVLYNSTSFSTFKCYGVSPSKLIDLCFTSVYADT | 98 |
| | | ACU31032_Rs_672_2006 | FLIRSSEVRQVAPGETGVIADYNYKLPDDFTGCVIAWNTAKQDQGQYYYRSSRKTKLKPFERDLTSDE | |
| | | | NGVRTLSTYDFYPNVPIEYQATRVVVLSFELLNAPATVCGPGSHHHHHH | |
| pMR14 | RBD insert | RBD-His6 from MZ081380_bat_ | NITNLCPFSQVFNATRFPSVYAWTRERISNCIADYSVLYNSTSFSTFRCYGVSPTKLNDLCFSNVYADS | 99 |
| | | Yunnan_RsYN04_2020 | MWVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFTSSVSQDYQPYRVVVLSFELLNAPATVCGPGSHHHHHH | |
| pMR14W | RBD insert | MR14 RBD-His6-T498W | NITNLCPFSQVFNATRFPSVYAWTRERISNCIADYSVLYNSTSFSTFRCYGVSPTKLNDLCFSNVYADS | 100 |
| | | | MWVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFWSSVSQDYQPYRVVVLSFELLNAPATVCGPGSHHHHHH | |
| pMR14YW | RBD insert | MR14 RBD-His6-L493Y-T498W | NITNLCPFSQVFNATRFPSVYAWTRERISNCIADYSVLYNSTSFSTFRCYGVSPTKLNDLCFSNVYADS | 101 |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLYKYGFWSSVSQDYQPYRVVVLSFELLNAPATVCGPGSHHHHHH | |
| pMR15 | RBD insert | Khosta2-MZ190138 | NMTNICPFDQVFNKTQFPSVYAWERVRISDCVSDYTVLYNSSASFSTFKCYGVSPTKLNDLCFSGVYA | 102 |
| | | | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFTSTSGVGYQPFRVVVLSFELLNAPATVCGPGSHHHHHH | |
| pMR15W | RBD insert | MR15 RBD-His6-T498W | NMTNICPFDQVFNKTQFPSVYAWERVRISDCVSDYTVLYNSSASFSTFKCYGVSPTKLNDLCFSGVYA | 103 |
| | | | DYFWKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFWSTSGVGYQPFRVVVLSFELLNAPATVCGPGSHHHHHH | |
| pMR15YW | RBD insert | MR15 RBD-His6-Q493Y-T498W | NMTNICPFDQVFNKTQFPSVYAWERVRISDCVSDYTVLYNSSASFSTFKCYGVSPTKLNDLCFSGVYA | 104 |
| | | | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLYDYGFWSTSGVGYQPFRVVVLSFELLNAPATVCGPGSHHHHHH | |
| pMR16 | RBD insert | KY352407_BtKY72_bat_Kenya | NITNLCPFGQVFNASNFPSVYAWERLRISDCVADYAVLYNSSSSFSTFKCYGVSPTKLNDLCFSSVYA | 105 |
| | | | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFTPTVGVGYQPYRVVVLSFELLNAPATVCGPGSHHHHH | |
| | | | H | |
| pMR16W | RBD insert | MR16 RBD-His6-T498W | NITNLCPFGQVFNASNFPSVYAWERLRISDCVADYAVLYNSSSSFSTFKCYGVSPTKLNDLCFSSVYA | 106 |
| | | | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFWPTVGVGYQPYRVVVLSFELLNAPATVCGPGSHHHH | |
| | | | HH | |
| pMR16YW | RBD insert | MR16 RBD-His6-K493Y-T498W | NITNLCPFGQVFNASNFPSVYAWERLRISDCVADYAVLYNSSSSFSTFKCYGVSPTKLNDLCFSSVYA | 107 |
| | | | DYFVVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLYSYGFWPTVGVGYQPYRVVVLSFELLNAPATVCGPGSHHHH | |
| | | | HH | |
| pMR17 | RBD insert | RaTG15_Ra7909 (RBD-His6) | NITNLCPFSQVFNATRFPSVYAWTRERISNCIADYSVLYNSTSFSTFRCYGVSPTKLNDLCFSNVYADS | 108 |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFTGSVSQDYQPYRVVVLSFELLNAPATVCGPGSHHHHHH | |
| pMR18 | RBD insert | HKU3-1_DQ022305.2005 | NITNRCPFDKVFNATRFPNVYAWERTKISDCVADYTVLYNSTSFSTFKCYGVSPSKLIDLCFTSVYADT | 109 |
| | | (RBD-His6) | FLIRSSEVRQVAPGETGVIADYNYKLPDDFTGCVIAWNTAKHDTGNYYYRSHRKTKLKPFERDLSSDD | |
| | | | GNGVYTLSTYDFNPNVPVAYQATRVVVLSFELLNAPATVCGPGSHHHHHH | |
| pMR19 | RBD insert | RhGB01-MW719567 | NVTQLCPFSDVFNRTMFPSVYAWERMSIRDCVADYAVLYNSSISFSTFECYGVSPTKLNDLCFSSVYA | 110 |
| | | | DYFWVRGDDVKQIAPGKTGVIADYNYKLPDDFVGCVLAWNSRHVDNKQGFYYRLFRHGKIKPYQRDM | |
| | | | SRELYSPSGTPCTVVGFNCYDPLAHYAFASPGIGIGYQPFRVVVLSFQLLNAPATVCGPGSHHHHHH | |
| pClu1 | RBD insert | bioinformatically derived | NITNLCPFDEVFNATRFPSVYAWERKKISNCVADYSVLYNSTSFSTFKCYGVSPSKLNDLCFTSVYADS | 111 |
| | | Cluster1_RBD | FVIRGDEVRQIAPGQTGVIADYNYKLPDDFTGCVIAWNTANQDSGEGGNPNYYYRSSRKSKLKPFER | |
| | | | DLSSDEYNVGSQPCNRMGPNCYRTLSTYDFYPTVGVGYQPYRVVVLSFELLNAPATVCGPGSHHHH | |
| | | | HH** | |
| pClu1a | RBD insert | Clu1RBD-His6-R355A | NITNLCPFDEVFNATRFPSVYAWEAKKISNCVADYSVLYNSTSFSTFKCYGVSPSKLNDLCFTSVYADS | 112 |
| | | (Class V knockout) | FVIRGDEVRQIAPGQTGVIADYNYKLPDDFTGCVIAWNTANQDSGEGGNPNYYYRSSRKSKLKPFER | |
| | | | DLSSDEYNVGSQPCNRMGPNCYRTLSTYDFYPTVGVGYQPYRVVVLSFELLNAPATVCGPGSHHHH | |
| | | | HH | |
| pClu1b | RBD insert | Clu1RBD-His6-K462A | NITNLCPFDEVFNATRFPSVYAWERKKISNCVADYSVLYNSTSFSTFKCYGVSPSKLNDLCFTSVYADS | 113 |
| | | (Class V knockout) | FVIRGDEVRQIAPGQTGVIADYNYKLPDDFTGCVIAWNTANQDSGEGGNPNYYYRSSRKSKLAPFER | |
| | | | DLSSDEYNVGSQPCNRMGPNCYRTLSTYDFYPTVGVGYQPYRVVVLSFELLNAPATVCGPGSHHHH | |
| | | | HH | |
| pClu1c | RBD insert | Clu1RBD-His6-R466A | NITNLCPFDEVFNATRFPSVYAWERKKISNCVADYSVLYNSTSFSTFKCYGVSPSKLNDLCFTSVYADS | 114 |
| | | (Class V knockout) | FVIRGDEVRQIAPGQTGVIADYNYKLPDDFTGCVIAWNTANQDSGEGGNPNYYYRSSRKSKLKPFEA | |
| | | | DLSSDEYNVGSQPCNRMGPNCYRTLSTYDFYPTVGVGYQPYRVVVLSFELLNAPATVCGPGSHHHH | |
| | | | HH | |
| pClu2 | RBD insert | bioinformatically derived | NLPDCNIEEWLTAKSVPSPLNWERKTFSNCNFNLSSLLRFVQADSFSCNNIDASKVYGMCFGSISVDK | 115 |
| | | Cluster2_RBD | FAIPNSRQVDLQLGNSGFLQSFNYKIDTTATSCQLYYSLPANNVTINNYNPSSWNRRYGFNDDTVFKP | |
| | | | RPAGVLTNHDVVYSQQCFKVPNTFCPCKNNGSCVSSGKGTTNGSGTCPAGTNYRTCENTTWCACS | |
| | | | CTPDPITASGPRKCPQKKSLVGVGEHCSGLGVDSDYCGNVSCLCSTDAFLGWSADSCLVNDRCNIFA | |
| | | | NIILNGINSGTTCSTGSHHHHHH** | |
| pClu3 | RBD insert | bioinformatically derived | QGVECDFSKLFKAAAPQIYNFSRLVFTNCNYNLTKLLSLFQVDEFSCHQISPSALASGCYSSLTVDYFA | 116 |
| | | Cluster3_RBD | YPLSMKSYLQIGSAGEISQFNYKQDFSNPTCRILASVPANVSITKPDKYIYISQCYSLSAYSDVDVPHYV | |
| | | | NPGQYTSIPCLYLTSSGFDEDGQYFRYNLTQFEGQNYLVATGVTVSMTDNLQMVFVITVQYGTDTNS | |
| | | | VCPGSHHHHHH** | |
| pOC43-F02 | OC43 SpFN | OC43-Fn (1b06v0-nogly) | AVIGDLKCTSDNINDKDTGPPPISTDTVDVTNGLGTYYVLDRVYLNTTLFLNGYYPTSGSTYRNMALKG | 117 |
| | | | SVLLSRLWFKPPFLSDFINGIFAKVKNTKVIKDRVMYSEFPAITIGSTFVNTSYSVWVQPRTINSTQDGD | |
| | | | NKLQGLLEVSVCQYNMCEYPQTICHPNLGNHRKELWHLDTGVVSCLYKRNFTYDVNADYLYFHFYQE | |
| | | | GGTFYAYFTDTGVVTKFLFNVYLGMALSHYYVMPLTCNSKLTLEYWVTPLTSRQYLLAFNQDGIIFNAV | |
| | | | DCMSDFMSEIKCKTQSIAPPTGVYELNGYTVQPIADVYRRKPNLPNCNIEAWLNDKSVPSPLNWERKT | |
| | | | FSNCNFNMSSLMSFIQADSFTCNNIDAAKIYGMCFSSITIDKFAIPNGRKVDLQLGNLGYLQSFNYRIDT | |
| | | | TATSCQLYYNLPAANVSVSRFNPSTWNKRFGFIEDSVFKPRPAGVLTNHDVVYAQHCFKAPKNFCPC | |
| | | | KLNGSCVGSGPGKNNGIGTCPAGTNYLTCDNLCTPDPITFTGTYKCPQTKSLVGIGEHCSGLAVKSDY | |
| | | | CGGNSCTCRPQAFLGWSADSCLQGDKCNIFANFILHDVNSGLTCSTDLQKANTDIILGVCVNYDLYGIL | |
| | | | GQGIFVEVNATYYNSWQNLLYDSNGNLYGFRDYITNRTFMIRSCYSGRVSAAFHANSSEPALLFRNIK | |
| | | | CNYVFNNSLTRQLQPINYFDSYLGCVVNAYNSTAISVQTCDLTVGSGYCVDYSKNGGSGGAITTGYRF | |
| | | | TNFEPFTVNSVNDSLEPVGGLYEIQIPSEFTIGNMVEFIQTSSPKVTIDCAAFVCGDYAACKSQLVEYGS | |
| | | | FCDNINAILTEVNELLDTTQLQVANSLMNGVTLSTKLKDGVNFNVDDINFSPVLGCLGSECSKASSRSAI | |
| | | | EDLLFDKVKLSDVGFVEAYNNCTGGAEIRDLICVQSYKGIKVLPPLLSENQFSGYTLAATSASLFPPWT | |
| | | | AAAGVPFYLNVQYRINGLGVTMDVLSQNQKLIANAFNNALYAIQEGFDATNSALVKIQAVVNANAEALN | |
| | | | NLLQQLSNRFGAISASLQEILSRLDPPEAEAQIDRLINGRLTALNAYVSQQLSDSTLVKFSAAQAMEKVN | |
| | | | ECVKSQSSRINFCGNGNHIISLVQNAPYGLYFIHFSYVPTKYVTARVSPGLCIAGDRGIAPKSGYFVNVN | |
| | | | NTWMYTGSGYYYPEPITENNVVVMSTCAVNYTKAPYVMLNTSISELQDFKEELDQWHKQGSGGSGDI | |
| | | | IKLLNEQVNKEMQSSNLYMSMSSWCYTHSLDGAGLFLFDHAAEEYEHAKKLIIFLNENNVPVQLTSISA | |
| | | | PEHKFEGLTQIFQKAYEHEQHISESINNIVDHAIKSKDHATFNFLQWYVAEQHEEEVLFKDILDKIELIGN | |
| | | | ENHGLYLADQYVKGIAKSRKSGS | |
| pOC43-F03 | OC43 SpFN | OC43-Fn (1b06v1) | AVIGDLKCTSDNINDKDTGPPPISTDTVDVTNGLGTYYVLDRVYLNTTLFLNGYYPTSGSTYRNMALKG | 118 |
| | | | SVLLSRLWFKPPFLSDFINGIFAKVKNTKVIKDRVMYSEFPAITIGSTFVNTSYSVVVQPRTINSTQDGD | |
| | | | NKLQGLLEVSVCQYNMCEYPQTICHPNLGNHRKELWHLDTGVVSCLYKRNFTYDVNADYLYFHFYQE | |
| | | | GGTFYAYFTDTGVVTKFLFNVYLGMALSHYYVMPLTCNSKLTLEYWVTPLTSRQYLLAFNQDGIIFNAV | |
| | | | DCMSDFMSEIKCKTQSIAPPTGVYELNGYTVQPIADVYRRKPNLPNCNIEAWLNDKSVPSPLNWERKT | |
| | | | FSNCNFNMSSLMSFIQADSFTCNNIDAAKIYGMCFSSITIDKFAIPNGRKVDLQLGNLGYLQSFNYRIDT | |
| | | | TATSCQLYYNLPAANVSVSRFNPSTWNKRFGFIEDSVFKPRPAGVLTNHDVVYAQHCFKAPKNFCPC | |
| | | | KLNGSCVGSGPGKNNGIGTCPAGTNYLTCDNLCTPDPITFTGTYKCPQTKSLVGIGEHCSGLAVKSDY | |
| | | | CGGNSCTCRPQAFLGWSADSCLQGDKCNIFANFILHDVNSGLTCSTDLQKANTDIILGVCVNYDLYGIL | |
| | | | GQGIFVEVNATYYNSWQNLLYDSNGNLYGFRDYITNRTFMIRSCYSGRVSAAFHANSSEPALLFRNIK | |
| | | | CNYVFNNSLTRQLQPINYFDSYLGCVVNAYNSTAISVQTCDLTVGSGYCVDYSKNGGSGGAITTGYRF | |
| | | | TNFEPFTVNSVNDSLEPVGGLYEIQIPSEFTIGNMVEFIQTSSPKVTIDCAAFVCGDYAACKSQLVEYGS | |
| | | | FCDNINAILTEVNELLDTTQLQVANSLMNGVTLSTKLKDGVNFNVDDINFSPVLGCLGSECSKASSRSAI | |
| | | | EDLLFDKVKLSDVGFVEAYNNCTGGAEIRDLICVQSYKGIKVLPPLLSENQFSGYTLAATSASLFPPWT | |
| | | | AAAGVPFYLNVQYRINGLGVTMDVLSQNQKLIANAFNNALYAIQEGFDATNSALVKIQAVVNANAEALN | |
| | | | NLLQQLSNRFGAISASLQEILSRLDPPEAEAQIDRLINGRLTALNAYVSQQLSDSTLVKFSAAQAMEKVN | |
| | | | ECVKSQSSRINFCGNGNHIISLVQNAPYGLYFIHFSYVPTKYVTARVSPGLCIAGDRGIAPKSGYFVNVN | |
| | | | NTWMYTGSGYYYPEPITENNVVVMSTCAVNYTKAPYVMLNTLQSNLQDIKEELDQIHKNGSGGSGDII | |
| | | | KLLNEQVNKEMQSSNLYMSMSSWCYTHSLDGAGLFLFDHAAEEYEHAKKLIIFLNENNVPVQLTSISAP | |
| | | | EHKFEGLTQIFQKAYEHEQHISESINNIVDHAIKSKDHATFNFLQWYVAEQHEEEVLFKDILDKIELIGNE | |
| | | | NHGLYLADQYVKGIAKSRKSGS | |
| pOC43-F04 | OC43 SpFN | OC43-Fn (1b06v1-nogly) | AVIGDLKCTSDNINDKDTGPPPISTDTVDVTNGLGTYYVLDRVYLNTTLFLNGYYPTSGSTYRNMALKG | 119 |
| | | | SVLLSRLWFKPPFLSDFINGIFAKVKNTKVIKDRVMYSEFPAITIGSTFVNTSYSVVVQPRTINSTQDGD | |
| | | | NKLQGLLEVSVCQYNMCEYPQTICHPNLGNHRKELWHLDTGVVSCLYKRNFTYDVNADYLYFHFYQE | |
| | | | GGTFYAYFTDTGVVTKFLFNVYLGMALSHYYVMPLTCNSKLTLEYWVTPLTSRQYLLAFNQDGIIFNAV | |
| | | | DCMSDFMSEIKCKTQSIAPPTGVYELNGYTVQPIADVYRRKPNLPNCNIEAWLNDKSVPSPLNWERKT | |
| | | | FSNCNFNMSSLMSFIQADSFTCNNIDAAKIYGMCFSSITIDKFAIPNGRKVDLQLGNLGYLQSFNYRIDT | |
| | | | TATSCQLYYNLPAANVSVSRFNPSTWNKRFGFIEDSVFKPRPAGVLTNHDVVYAQHCFKAPKNFCPC | |
| | | | KLNGSCVGSGPGKNNGIGTCPAGTNYLTCDNLCTPDPITFTGTYKCPQTKSLVGIGEHCSGLAVKSDY | |
| | | | CGGNSCTCRPQAFLGWSADSCLQGDKCNIFANFILHDVNSGLTCSTDLQKANTDIILGVCVNYDLYGIL | |
| | | | GQGIFVEVNATYYNSWQNLLYDSNGNLYGFRDYITNRTFMIRSCYSGRVSAAFHANSSEPALLFRNIK | |
| | | | CNYVFNNSLTRQLQPINYFDSYLGCVVNAYNSTAISVQTCDLTVGSGYCVDYSKNGGSGGAITTGYRF | |
| | | | TNFEPFTVNSVNDSLEPVGGLYEIQIPSEFTIGNMVEFIQTSSPKVTIDCAAFVCGDYAACKSQLVEYGS | |
| | | | FCDNINAILTEVNELLDTTQLQVANSLMNGVTLSTKLKDGVNFNVDDINFSPVLGCLGSECSKASSRSAI | |
| | | | EDLLFDKVKLSDVGFVEAYNNCTGGAEIRDLICVQSYKGIKVLPPLLSENQFSGYTLAATSASLFPPWT | |
| | | | AAAGVPFYLNVQYRINGLGVTMDVLSQNQKLIANAFNNALYAIQEGFDATNSALVKIQAVVNANAEALN | |
| | | | NLLQQLSNRFGAISASLQEILSRLDPPEAEAQIDRLINGRLTALNAYVSQQLSDSTLVKFSAAQAMEKVN | |
| | | | ECVKSQSSRINFCGNGNHIISLVQNAPYGLYFIHFSYVPTKYVTARVSPGLCIAGDRGIAPKSGYFVNVN | |
| | | | NTWMYTGSGYYYPEPITENNVVVMSTCAVNYTKAPYVMLNTLQSNLQDIKEELDQIHKQGSGGSGDII | |
| | | | KLLNEQVNKEMQSSNLYMSMSSWCYTHSLDGAGLFLFDHAAEEYEHAKKLIIFLNENNVPVQLTSISAP | |
| | | | EHKFEGLTQIFQKAYEHEQHISESINNIVDHAIKSKDHATFNFLQWYVAEQHEEEVLFKDILDKIELIGNE | |
| | | | NHGLYLADQYVKGIAKSRKSGS | |
| pOC43-F05 | OC43 SpFN | OC43-Fn (1b06v2) | AVIGDLKCTSDNINDKDTGPPPISTDTVDVTNGLGTYYVLDRVYLNTTLFLNGYYPTSGSTYRNMALKG | 120 |
| | | | SVLLSRLWFKPPFLSDFINGIFAKVKNTKVIKDRVMYSEFPAITIGSTFVNTSYSVVVQPRTINSTQDGD | |
| | | | NKLQGLLEVSVCQYNMCEYPQTICHPNLGNHRKELWHLDTGVVSCLYKRNFTYDVNADYLYFHFYQE | |
| | | | GGTFYAYFTDTGVVTKFLFNVYLGMALSHYYVMPLTCNSKLTLEYWVTPLTSRQYLLAFNQDGIIFNAV | |
| | | | DCMSDFMSEIKCKTQSIAPPTGVYELNGYTVQPIADVYRRKPNLPNCNIEAWLNDKSVPSPLNWERKT | |
| | | | FSNCNFNMSSLMSFIQADSFTCNNIDAAKIYGMCFSSITIDKFAIPNGRKVDLQLGNLGYLQSFNYRIDT | |
| | | | TATSCQLYYNLPAANVSVSRFNPSTWNKRFGFIEDSVFKPRPAGVLTNHDVVYAQHCFKAPKNFCPC | |
| | | | KLNGSCVGSGPGKNNGIGTCPAGTNYLTCDNLCTPDPITFTGTYKCPQTKSLVGIGEHCSGLAVKSDY | |
| | | | CGGNSCTCRPQAFLGWSADSCLQGDKCNIFANFILHDVNSGLTCSTDLQKANTDIILGVCVNYDLYGIL | |
| | | | GQGIFVEVNATYYNSWQNLLYDSNGNLYGFRDYITNRTFMIRSCYSGRVSAAFHANSSEPALLFRNIK | |
| | | | CNYVFNNSLTRQLQPINYFDSYLGCVVNAYNSTAISVQTCDLTVGSGYCVDYSKNGGSGGAITTGYRF | |
| | | | TNFEPFTVNSVNDSLEPVGGLYEIQIPSEFTIGNMVEFIQTSSPKVTIDCAAFVCGDYAACKSQLVEYGS | |
| | | | FCDNINAILTEVNELLDTTQLQVANSLMNGVTLSTKLKDGVNFNVDDINFSPVLGCLGSECSKASSRSAI | |
| | | | EDLLFDKVKLSDVGFVEAYNNCTGGAEIRDLICVQSYKGIKVLPPLLSENQFSGYTLAATSASLFPPWT | |
| | | | AAAGVPFYLNVQYRINGLGVTMDVLSQNQKLIANAFNNALYAIQEGFDATNSALVKIQAVVNANAEALN | |
| | | | NLLQQLSNRFGAISASLQEILSRLDPPEAEAQIDRLINGRLTALNAYVSQQLSDSTLVKFSAAQAMEKVN | |
| | | | ECVKSQSSRINFCGNGNHIISLVQNAPYGLYFIHFSYVPTKYVTARVSPGLCIAGDRGIAPKSGYFVNVN | |
| | | | NTWMYTGSGYYYPEPITENNVVVMSTCAVNYTKAPYVMLNTLQSNLQDLKEELDQLHKNGSGGSGDI | |
| | | | IKLLNEQVNKEMQSSNLYMSMSSWCYTHSLDGAGLFLFDHAAEEYEHAKKLIIFLNENNVPVQLTSISA | |
| | | | PEHKFEGLTQIFQKAYEHEQHISESINNIVDHAIKSKDHATFNFLQWYVAEQHEEEVLFKDILDKIELIGN | |
| | | | ENHGLYLADQYVKGIAKSRKSGS | |
| pOC43-F06 | OC43 SpFN | OC43-Fn (1b06v2-nogly) | AVIGDLKCTSDNINDKDTGPPPISTDTVDVTNGLGTYYVLDRVYLNTTLFLNGYYPTSGSTYRNMALKG | 121 |
| | | | SVLLSRLWFKPPFLSDFINGIFAKVKNTKVIKDRVMYSEFPAITIGSTFVNTSYSVVVQPRTINSTQDGD | |
| | | | NKLQGLLEVSVCQYNMCEYPQTICHPNLGNHRKELWHLDTGVVSCLYKRNFTYDVNADYLYFHFYQE | |
| | | | GGTFYAYFTDTGVVTKFLFNVYLGMALSHYYVMPLTCNSKLTLEYWVTPLTSRQYLLAFNQDGIIFNAV | |
| | | | DCMSDFMSEIKCKTQSIAPPTGVYELNGYTVQPIADVYRRKPNLPNCNIEAWLNDKSVPSPLNWERKT | |
| | | | FSNCNFNMSSLMSFIQADSFTCNNIDAAKIYGMCFSSITIDKFAIPNGRKVDLQLGNLGYLQSFNYRIDT | |
| | | | TATSCQLYYNLPAANVSVSRFNPSTWNKRFGFIEDSVFKPRPAGVLTNHDVVYAQHCFKAPKNFCPC | |
| | | | KLNGSCVGSGPGKNNGIGTCPAGTNYLTCDNLCTPDPITFTGTYKCPQTKSLVGIGEHCSGLAVKSDY | |
| | | | CGGNSCTCRPQAFLGWSADSCLQGDKCNIFANFILHDVNSGLTCSTDLQKANTDIILGVCVNYDLYGIL | |
| | | | GQGIFVEVNATYYNSWQNLLYDSNGNLYGFRDYITNRTFMIRSCYSGRVSAAFHANSSEPALLFRNIK | |
| | | | CNYVFNNSLTRQLQPINYFDSYLGCVVNAYNSTAISVQTCDLTVGSGYCVDYSKNGGSGGAITTGYRF | |
| | | | TNFEPFTVNSVNDSLEPVGGLYEIQIPSEFTIGNMVEFIQTSSPKVTIDCAAFVCGDYAACKSQLVEYGS | |
| | | | FCDNINAILTEVNELLDTTQLQVANSLMNGVTLSTKLKDGVNFNVDDINFSPVLGCLGSECSKASSRSAI | |
| | | | EDLLFDKVKLSDVGFVEAYNNCTGGAEIRDLICVQSYKGIKVLPPLLSENQFSGYTLAATSASLFPPWT | |
| | | | AAAGVPFYLNVQYRINGLGVTMDVLSQNQKLIANAFNNALYAIQEGFDATNSALVKIQAVVNANAEALN | |
| | | | NLLQQLSNRFGAISASLQEILSRLDPPEAEAQIDRLINGRLTALNAYVSQQLSDSTLVKFSAAQAMEKVN | |
| | | | ECVKSQSSRINFCGNGNHIISLVQNAPYGLYFIHFSYVPTKYVTARVSPGLCIAGDRGIAPKSGYFVNVN | |
| | | | NTWMYTGSGYYYPEPITENNVVVMSTCAVNYTKAPYVMLNTLQSNLQDLKEELDQLHKQGSGGSGDI | |
| | | | IKLLNEQVNKEMQSSNLYMSMSSWCYTHSLDGAGLFLFDHAAEEYEHAKKLIIFLNENNVPVQLTSISA | |
| | | | PEHKFEGLTQIFQKAYEHEQHISESINNIVDHAIKSKDHATFNFLQWYVAEQHEEEVLFKDILDKIELIGN | |
| | | | ENHGLYLADQYVKGIAKSRKSGS | |
| pOC43-F07 | OC43 SpFN | OC43-Fn (1b06v3-nogly) | AVIGDLKCTSDNINDKDTGPPPISTDTVDVTNGLGTYYVLDRVYLNTTLFLNGYYPTSGSTYRNMALKG | 122 |
| | | | SVLLSRLWFKPPFLSDFINGIFAKVKNTKVIKDRVMYSEFPAITIGSTFVNTSYSVVVQPRTINSTQDGD | |
| | | | NKLQGLLEVSVCQYNMCEYPQTICHPNLGNHRKELWHLDTGVVSCLYKRNFTYDVNADYLYFHFYQE | |
| | | | GGTFYAYFTDTGVVTKFLFNVYLGMALSHYYVMPLTCNSKLTLEYWVTPLTSRQYLLAFNQDGIIFNAV | |
| | | | DCMSDFMSEIKCKTQSIAPPTGVYELNGYTVQPIADVYRRKPNLPNCNIEAWLNDKSVPSPLNWERKT | |
| | | | FSNCNFNMSSLMSFIQADSFTCNNIDAAKIYGMCFSSITIDKFAIPNGRKVDLQLGNLGYLQSFNYRIDT | |
| | | | TATSCQLYYNLPAANVSVSRFNPSTWNKRFGFIEDSVFKPRPAGVLTNHDVVYAQHCFKAPKNFCPC | |
| | | | KLNGSCVGSGPGKNNGIGTCPAGTNYLTCDNLCTPDPITFTGTYKCPQTKSLVGIGEHCSGLAVKSDY | |
| | | | CGGNSCTCRPQAFLGWSADSCLQGDKCNIFANFILHDVNSGLTCSTDLQKANTDIILGVCVNYDLYGIL | |
| | | | GQGIFVEVNATYYNSWQNLLYDSNGNLYGFRDYITNRTFMIRSCYSGRVSAAFHANSSEPALLFRNIK | |
| | | | CNYVFNNSLTRQLQPINYFDSYLGCVVNAYNSTAISVQTCDLTVGSGYCVDYSKNGGSGGAITTGYRF | |
| | | | TNFEPFTVNSVNDSLEPVGGLYEIQIPSEFTIGNMVEFIQTSSPKVTIDCAAFVCGDYAACKSQLVEYGS | |
| | | | FCDNINAILTEVNELLDTTQLQVANSLMNGVTLSTKLKDGVNFNVDDINFSPVLGCLGSECSKASSRSAI | |
| | | | EDLLFDKVKLSDVGFVEAYNNCTGGAEIRDLICVQSYKGIKVLPPLLSENQFSGYTLAATSASLFPPWT | |
| | | | AAAGVPFYLNVQYRINGLGVTMDVLSQNQKLIANAFNNALYAIQEGFDATNSALVKIQAVVNANAEALN | |
| | | | NLLQQLSNRFGAISASLQEILSRLDPPEAEAQIDRLINGRLTALNAYVSQQLSDSTLVKFSAAQAMEKVN | |
| | | | ECVKSQSSRINFCGNGNHIISLVQNAPYGLYFIHFSYVPTKYVTARVSPGLCIAGDRGIAPKSGYFVNVN | |
| | | | NTWMYTGSGYYYPEPITENNVVVMSTCAVNYTKAPYVMLNTSISELQDIKEELDQIHKQGSGGSGDIIK | |
| | | | LLNEQVNKEMQSSNLYMSMSSWCYTHSLDGAGLFLFDHAAEEYEHAKKLIIFLNENNVPVQLTSISAPE | |
| | | | HKFEGLTQIFQKAYEHEQHISESINNIVDHAIKSKDHATFNFLQWYVAEQHEEEVLFKDILDKIELIGNEN | |
| | | | HGLYLADQYVKGIAKSRKSGS | |
| pOC43-F08 | OC43 SpFN | OC43-Fn (1b06v4-nogly) | AVIGDLKCTSDNINDKDTGPPPISTDTVDVTNGLGTYYVLDRVYLNTTLFLNGYYPTSGSTYRNMALKG | 123 |
| | | | SVLLSRLWFKPPFLSDFINGIFAKVKNTKVIKDRVMYSEFPAITIGSTFVNTSYSVVVQPRTINSTQDGD | |
| | | | NKLQGLLEVSVCQYNMCEYPQTICHPNLGNHRKELWHLDTGVVSCLYKRNFTYDVNADYLYFHFYQE | |
| | | | GGTFYAYFTDTGVVTKFLFNVYLGMALSHYYVMPLTCNSKLTLEYWVTPLTSRQYLLAFNQDGIIFNAV | |
| | | | DCMSDFMSEIKCKTQSIAPPTGVYELNGYTVQPIADVYRRKPNLPNCNIEAWLNDKSVPSPLNWERKT | |
| | | | FSNCNFNMSSLMSFIQADSFTCNNIDAAKIYGMCFSSITIDKFAIPNGRKVDLQLGNLGYLQSFNYRIDT | |
| | | | TATSCQLYYNLPAANVSVSRFNPSTWNKRFGFIEDSVFKPRPAGVLTNHDVVYAQHCFKAPKNFCPC | |
| | | | KLNGSCVGSGPGKNNGIGTCPAGTNYLTCDNLCTPDPITFTGTYKCPQTKSLVGIGEHCSGLAVKSDY | |
| | | | CGGNSCTCRPQAFLGWSADSCLQGDKCNIFANFILHDVNSGLTCSTDLQKANTDIILGVCVNYDLYGIL | |
| | | | GQGIFVEVNATYYNSWQNLLYDSNGNLYGFRDYITNRTFMIRSCYSGRVSAAFHANSSEPALLFRNIK | |
| | | | CNYVFNNSLTRQLQPINYFDSYLGCVVNAYNSTAISVQTCDLTVGSGYCVDYSKNGGSGGAITTGYRF | |
| | | | TNFEPFTVNSVNDSLEPVGGLYEIQIPSEFTIGNMVEFIQTSSPKVTIDCAAFVCGDYAACKSQLVEYGS | |
| | | | FCDNINAILTEVNELLDTTQLQVANSLMNGVTLSTKLKDGVNFNVDDINFSPVLGCLGSECSKASSRSAI | |
| | | | EDLLFDKVKLSDVGFVEAYNNCTGGAEIRDLICVQSYKGIKVLPPLLSENQFSGYTLAATSASLFPPWT | |
| | | | AAAGVPFYLNVQYRINGLGVTMDVLSQNQKLIANAFNNALYAIQEGFDATNSALVKIQAVVNANAEALN | |
| | | | NLLQQLSNRFGAISASLQEILSRLDPPEAEAQIDRLINGRLTALNAYVSQQLSDSTLVKFSAAQAMEKVN | |
| | | | ECVKSQSSRINFCGNGNHIISLVQNAPYGLYFIHFSYVPTKYVTARVSPGLCIAGDRGIAPKSGYFVNVN | |
| | | | NTWMYTGSGYYYPEPITENNVVVMSTCAVNYTKAPYVMLNTSISELQDLKEELDQLHKQGSGGSGDII | |
| | | | KLLNEQVNKEMQSSNLYMSMSSWCYTHSLDGAGLFLFDHAAEEYEHAKKLIIFLNENNVPVQLTSISAP | |
| | | | EHKFEGLTQIFQKAYEHEQHISESINNIVDHAIKSKDHATFNFLQWYVAEQHEEEVLFKDILDKIELIGNE | |
| | | | NHGLYLADQYVKGIAKSRKSGS | |
| pCoV256 | RBD-NTD-Ferritin | His8-3c-RBD-GSGGSG- | HHHHHHHHGPLEVLFQGPNITNLCPFGEVFNATRFASVYAWNRKRISNCVADYSVLYNSASFSTFKCY | 124 |
| RBD- | | NTD(delta)-Ferritin | GVSPTKLNDLCFTNVYADSFVIRGDEVRQIAPGQTGKIADYNYKLPDDFTGCVIAWNSNNLDSKVGGN | |
| NTD(delta)- | | | YNYLRRLFRKSNLKPFERDISTEIYQAGSTPCNGVEGFNCYFPLQSYGFQPTNGVGYQPYRVVVLSFE | |
| Ferritin | | | NKSAPATVCGPGSGGSGSSQCVNLRTRTQLPPAYTNSFTRGVYYPDKVFRSSVLHSTQDLFLPFFSN | |
| | | | VTWFHAIHVSGTNGTKRFDNPVLPFNDGVYFASIEKSNIIRGWIFGTTLDSKTQSLLIVNNATNVVIKVC | |
| | | | EFQFCNDPFLDVYYHKNNKSWMESGVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLGSGGGGESQVRQQFSKDIEKLLNEQVNKEMQSSN | |
| | | | LYMSMSSWCYTHSLDGAGLFLFDHAAEEYEHAKKLIIFLNENNVPVQLTSISAPEHKFEGLTQIFQKAY | |
| | | | EHEQHISESINNIVDHAIKSKDHATFNFLQWYVAEQHEEEVLFKDILDKIELIGNENHGLYLADQYVKGIA | |
| | | | KSRKSGS | |
| pNL63-F02 | NL63 SpFN | NL63-Fn (1b06v1) | CNSNANLSMLQLGVPDNSSTIVTGLLPTHWFCANQSTSVYSANGFFYIDVGNHRSAFALHTGYYDAN | 125 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSWVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WVDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYIGDLNKTLQELEQNLEKLVKKGSGGSGDIIKLLNEQVNKEMQSSNLYM | |
| | | | SMSSWCYTHSLDGAGLFLFDHAAEEYEHAKKLIIFLNENNVPVQLTSISAPEHKFEGLTQIFQKAYEHE | |
| | | | QHISESINNIVDHAIKSKDHATFNFLQWYVAEQHEEEVLFKDILDKIELIGNENHGLYLADQYVKGIAKSR | |
| | | | KSGS | |
| pNL63-F03 | NL63 SpFN | NL63-Fn (SARS2graft) | CNSNANLSMLQLGVPDNSSTIVTGLLPTHWFCANQSTSVYSANGFFYIDVGNHRSAFALHTGYYDAN | 126 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSVVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WVDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYVDINKTLQEIAQNLDKIHKQGSGGSGDIIKLLNEQVNKEMQSSNLYMSM | |
| | | | SSWCYTHSLDGAGLFLFDHAAEEYEHAKKLIIFLNENNVPVQLTSISAPEHKFEGLTQIFQKAYEHEQHI | |
| | | | SESINNIVDHAIKSKDHATFNFLQWYVAEQHEEEVLFKDILDKIELIGNENHGLYLADQYVKGIAKSRKS | |
| | | | GS | |
| pNL63-F04 | NL63 SpFN | NL63-Fn (MERSvOlike) | CNSNANLSMLQLGVPDNSSTIVTGLLPTHWFCANQSTSVYSANGFFYIDVGNHRSAFALHTGYYDAN | 127 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSVVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYVDLNKTLQEIAQQGSGGSGDIIKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| pNL63-F05 | NL63 SpFN | NL63(SARS2graft-linker) | CNSNANLSMLQLGVPDNSSTIVTGLLPTHWFCANQSTSVYSANGFFYIDVGNHRSAFALHTGYYDAN | 128 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSVVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYTGSGDINKTLQEIAQNLDKIHKQGSGGSGDIIKLLNEQVNKEMQSSNLY | |
| | | | MSMSSWCYTHSLDGAGLFLFDHAAEEYEHAKKLIIFLNENNVPVQLTSISAPEHKFEGLTQIFQKAYEH | |
| | | | EQHISESINNIVDHAIKSKDHATFNFLQWYVAEQHEEEVLFKDILDKIELIGNENHGLYLADQYVKGIAKS | |
| | | | RKSGS | |
| pNL63-F06 | NL63 SpFN | NL63 (MERSvOlike-linker) | CNSNANLSMLQLGVPDNSSTIVTGLLPTHWFCANQSTSVYSANGFFYIDVGNHRSAFALHTGYYDAN | 129 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSVVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WVDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYTGSGDLNKTLQEIAQQGSGGSGDIIKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| pNL63-F07 | NL63 SpFN | NL63(1b06v0-linker) | CNSNANLSMLQLGVPDNSSTIVTGLLPTHWFCANQSTSVYSANGFFYIDVGNHRSAFALHTGYYDAN | 130 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSVVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYTGSGDLNKTLQEIEQNLEKLDKKGSGGSGDIIKLLNEQVNKEMQSSNL | |
| | | | YMSMSSWCYTHSLDGAGLFLFDHAAEEYEHAKKLIIFLNENNVPVQLTSISAPEHKFEGLTQIFQKAYE | |
| | | | HEQHISESINNIVDHAIKSKDHATFNFLQWYVAEQHEEEVLFKDILDKIELIGNENHGLYLADQYVKGIAK | |
| | | | SRKSGS | |
| pNL63-F08 | NL63 SpFN | NL63(HR2graft) | CNSNANLSMLQLGVPDNSSTIVTGLLPTHWFCANQSTSVYSANGFFYIDVGNHRSAFALHTGYYDAN | 131 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSVVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYVDLNKTLQEIEQNLEKIDKKLNDLTKNLQNLSSELKQLEAKIDQINSTGS | |
| | | | GGSGDIIKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pNL63-F09 | NL63 SpFN | NL63(HR2graft-linker) | CNSNANLSMLQLGVPDNSSTIVTGLLPTHWFCANQSTSVYSANGFFYIDVGNHRSAFALHTGYYDAN | 132 |
| | | | QYYIYVTNEIGLNASVTLKICKFSRNTTFDFLSNASSSFDCIVNLLFTEQLGAPLGITISGETVRLHLYNVT | |
| | | | RTFYVPAAYKLTKLSVKCYFNYSCVFSVVNATVTVNVTTHNGRVVNYTVCDDCNGYTDNIFSVQQDG | |
| | | | RIPNGFPFNNWFLLTNGSTLVDGVSRLYQPLRLTCLWPVPGLKSSTGFVYFNATGSDVNCNGYQHNS | |
| | | | WVDVMRYNLNFSANSLDNLKSGVIVFKTLQYDVLFYCSNSSSGVLDTTIPFGPSSQPYYCFINSTINTTH | |
| | | | VSTFVGILPPTVREIVVARTGQFYINGFKYFDLGFIEAVNFNVTTASATDFWTVAFATFVDVLVNVSATN | |
| | | | IQNLLYCDSPFEKLQCEHLQFGLQDGFYSANFLDDNVLPETYVALPIYYQHTDINFTATASFGGSCYVC | |
| | | | KPHQVNISLNGNTSVCVRTSHFSIRYIYNRVKSGSPGDSSWHIYLKSGTCPFSFSKLNNFQKFKTICFS | |
| | | | TVEVPGSCNFPLEATWHYTSYTIVGALYVTWSEGNSITGVPYPVSGIREFSNLVLNNCTKYNIYDYVGT | |
| | | | GIIRSSNQSLAGGITYVSNSGNLLGFKNVSTGNIFIVTPCNQPDQVAVYQQSIIGAMTAVNESRYGLQNL | |
| | | | LQLPNFYYVSNGGNNCTTAVMTYSNFGICADGSLIPVRPRNSSDNGISAIITANLSIPSNWTTSVQVEYL | |
| | | | QITSTPIVVDCATYVCNGNPRCKNLLKQYTSACKTIEDALRLSAHLETNDVSSMLTFDSNAFSLANVTSF | |
| | | | GDYNLSSVLPQRNIRSSRIAGRSALEDLLFSKVVTSGLGTVDVDYKSCTKGLSIADLACAQYYNGIMVL | |
| | | | PGVADAERMAMYTGSLIGGMVLGGLTSAAAIPFSLALQARLNYVALQTDVLQENQKILAASFNKAINNI | |
| | | | VASFSSVNDAITQTAEAIHTVTIALNKIQDVVNQQGSALNHLTSQLRHNFQAISNSIQAIYDRLDPPQAD | |
| | | | QQVDRLITGRLAALNAFVSQVLNKYTEVRGSRRLAQQKINECVKSQSNRYGFCGNGTHIFSIVNSAPD | |
| | | | GLLFLHTVLLPTDYKNVKAWSGICVDGIYGYVLRQPNLVLYSDNGVFRVTSRVMFQPRLPVLSDFVQIY | |
| | | | NCNVTFVNISRVELHTVIPDYTGSGDLNKTLQEIEQNLEKIDKKLNDLTKNLQNLSSELKQLEAKIDQINS | |
| | | | TGSGGSGDIIKLLNEQVNKEMQSSNLYMSMSSWCYTHSLDGAGLFLFDHAAEEYEHAKKLIIFLNENN | |
| | | | VPVQLTSISAPEHKFEGLTQIFQKAYEHEQHISESINNIVDHAIKSKDHATFNFLQWYVAEQHEEEVLFK | |
| | | | DILDKIELIGNENHGLYLADQYVKGIAKSRKSGS | |
| pHKU1-F01 | mosaic SpFN | HKU1 SpFN with OC43 RBD | VIGDFNCTNSFINDYNKTIPRISEDWDVSLGLGTYYVLNRVYLNTTLLFTGYFPKSGANFRDLALKGSIY | 133 |
| | | replacement | LSTLWYKPPFLSDFNNGIFSKVKNTKLYVNNTLYSEFSTIVIGSVFVNTSYTIVVQPHNGILEITACQYTM | |
| | | | CEYPHTVCKSKGSIRNESWHIDSSEPLCLFKKNFTYNVSADWLYFHFYQERGVFYAYYADVGMPTTF | |
| | | | LFSLYLGTILSHYYVMPLTCNAISSNTDNETLEYWVTPLSRRQYLLNFDEHGVITNAVDCSSSFLSEIQC | |
| | | | KTQSFAPNTGVYDLSGFTVKPVATVYRRIPNLPGSNCNIEAWLNDKSVPSPLNWERKTFSNCNFNMS | |
| | | | SLMSFIQADSFTCNNIDAAKIYGMCFSSITIDKFAIPNGRKVDLQLGNLGYLQSFNYRIDTTATSCQLYYN | |
| | | | LPAANVSVSRFNPSTWNKRFGFIEDSVFKPRPAGVLTNHDVVYAQHCFKAPKNFCPCKLNGSCVGSG | |
| | | | PGKNNGIGTCPAGTNYLTCDNLCTPDPITFTGTYKCPQTKSLVGIGEHCSGLAVKSDYCGGNSCTCRP | |
| | | | QAFLGWSADSCLQGDKCNIFANFILHDVNSGLTCSTDGSDLLYSNTEISTGVCVNYDLYGITGQGIFKE | |
| | | | VSAAYYNNWQNLLYDSNGNIIGFKDFLINKTYTILPCYSGRVSAAFYQNSSSPALLYRNLKCSYVLNNI | |
| | | | SFISQPFYFDSYLGCVLNAVNLTSYSVSSCDLRMGSGFCIDYALPSSGGSGSGISSPYRFVTFEPFNVS | |
| | | | FVNDSVETVGGLFEIQIPTNFTIAGHEEFIQTSSPKVTIDCSAFVCSNYAACHDLLSEYGTFCDNINSILN | |
| | | | EVNDLLDITQLQVANALMQGVTLSSNLNTNLHSDVDNIDFKSLLGCLGSQCGSSSRSLLEDLLFNKVKL | |
| | | | SDVGFVEAYNNCTGGSEIRDLLCVQSFNGIKVLPPILSETQISGYTTAATVAAMFPPWSAAAGVPFSLN | |
| | | | VQYRINGLGVTMDVLNKNQKLIANAFNKALLSIQNGFTATNSALAKIQSVVNANAQALNSLLQQLFNKF | |
| | | | GAISSSLQEILSRLDPPEAQVQIDRLINGRLTALNAYVSQQLSDITLIKAGASRAIEKVNECVKSQSPRINF | |
| | | | CGNGNHILSLVQNAPYGLLFIHFSYKPTSFKTVLVSPGLCLSGDRGIAPKQGYFIKQNDSWMFTGSSY | |
| | | | YYPEPISDKNVVFMNSCSVNFTKAPFIYLNNSISELSDFEAELSQWHKNGSGGSGDIIKLLNEQVNKEM | |
| | | | QSSNLYMSMSSWCYTHSLDGAGLFLFDHAAEEYEHAKKLIIFLNENNVPVQLTSISAPEHKFEGLTQIF | |
| | | | QKAYEHEQHISESINNIVDHAIKSKDHATFNFLQWYVAEQHEEEVLFKDILDKIELIGNENHGLYLADQY | |
| | | | VKGIAKSRKSGS | |
| pHKU1-F02 | mosaic SpFN | HKU1 SpFN with OC43 | VIGDFNCTNSFINDYNKTIPRISEDVVDVSLGLGTYYVLNRVYLNTTLLFTGYFPKSGANFRDLALKGSIY | 134 |
| | | RBD-SD1 replacement | LSTLWYKPPFLSDFNNGIFSKVKNTKLYVNNTLYSEFSTIVIGSVFVNTSYTIVVQPHNGILEITACQYTM | |
| | | | CEYPHTVCKSKGSIRNESWHIDSSEPLCLFKKNFTYNVSADWLYFHFYQERGVFYAYYADVGMPTTF | |
| | | | LFSLYLGTILSHYYVMPLTCNAISSNTDNETLEYWVTPLSRRQYLLNFDEHGVITNAVDCSSSFLSEIQC | |
| | | | KTQSFAPNTGVYDLSGFTVKPVGSQPIADVYRRKPNLPNCNIEAWLNDKSVPSPLNWERKTFSNCNF | |
| | | | NMSSLMSFIQADSFTCNNIDAAKIYGMCFSSITIDKFAIPNGRKVDLQLGNLGYLQSFNYRIDTTATSCQ | |
| | | | LYYNLPAANVSVSRFNPSTWNKRFGFIEDSVFKPRPAGVLTNHDVVYAQHCFKAPKNFCPCKLNGSC | |
| | | | VGSGPGKNNGIGTCPAGTNYLTCDNLCTPDPITFTGTYKCPQTKSLVGIGEHCSGLAVKSDYCGGNS | |
| | | | CTCRPQAFLGWSADSCLQGDKCNIFANFILHDVNSGLTCSTDLQKANTDIILGVCVNYDLYGILGQGIF | |
| | | | VEVNATYYNSWQNLLYDSNGNLYGFRDYITNRTFMIRSCGSPCYSGRVSAAFYQNSSSPALLYRNLK | |
| | | | CSYVLNNISFISQPFYFDSYLGCVLNAVNLTSYSVSSCDLRMGSGFCIDYALPSSGGSGSGISSPYRFV | |
| | | | TFEPFNVSFVNDSVETVGGLFEIQIPTNFTIAGHEEFIQTSSPKVTIDCSAFVCSNYAACHDLLSEYGTF | |
| | | | CDNINSILNEVNDLLDITQLQVANALMQGVTLSSNLNTNLHSDVDNIDFKSLLGCLGSQCGSSSRSLLED | |
| | | | LLFNKVKLSDVGFVEAYNNCTGGSEIRDLLCVQSFNGIKVLPPILSETQISGYTTAATVAAMFPPWSAA | |
| | | | AGVPFSLNVQYRINGLGVTMDVLNKNQKLIANAFNKALLSIQNGFTATNSALAKIQSVVNANAQALNSLL | |
| | | | QQLFNKFGAISSSLQEILSRLDPPEAQVQIDRLINGRLTALNAYVSQQLSDITLIKAGASRAIEKVNECVK | |
| | | | SQSPRINFCGNGNHILSLVQNAPYGLLFIHFSYKPTSFKTVLVSPGLCLSGDRGIAPKQGYFIKQNDSW | |
| | | | MFTGSSYYYPEPISDKNVVFMNSCSVNFTKAPFIYLNNSISELSDFEAELSQWHKNGSGGSGDIIKLLN | |
| | | | EQVNKEMQSSNLYMSMSSWCYTHSLDGAGLFLFDHAAEEYEHAKKLIIFLNENNVPVQLTSISAPEHK | |
| | | | FEGLTQIFQKAYEHEQHISESINNIVDHAIKSKDHATFNFLQWYVAEQHEEEVLFKDILDKIELIGNENHG | |
| | | | LYLADQYVKGIAKSRKSGS | |
| p229E-F01 | mosaic SpFN | 229E SpFN with NL63 RBD | CQTTNGLNTSYSVCNGCVGYSENVFAVESGGYIPSDFAFNNWFLLTNTSSVVDGVVRSFQPLLLNCL | 135 |
| | | replacement | WSVSGLRFTTGFVYFNGTGRGDCKGFSSDVLSDVIRYNLNFEENLRRGTILFKTSYGVVVFYCTNNTL | |
| | | | VSGDAHIPFGTVLGNFYCFVNTTIGNETTSAFVGALPKTVREFVISRTGHFYINGYRYFTLGNVEAVNF | |
| | | | NVTTAETTDFCTVALASYADVLVNVSQTSIANIIYCNSVINRLRCDQLSFDVPDGFYSTSPIQSVELPVSI | |
| | | | VSLPVYHGSQHTDINFTATASFGGSCYVCKPHQVNISLNGNTSVCVRTSHFSIRYIYNRVKSGSPGDS | |
| | | | SWHIYLKSGTCPFSFSKLNNFQKFKTICFSTVEVPGSCNFPLEATWHYTSYTIVGALYVTWSEGNSITG | |
| | | | GSGVPQPVEGVSSFMNVTLDKCTKYNIYDVSGVGVIRVSNDTFLNGITYTSTSGNLLGFKDVTKGTIYS | |
| | | | ITPCNPPDQLVVYQQAVVGAMLSENFTSYGFSNVVELPKFFYASNGTYNCTDAVLTYSSFGVCADGSII | |
| | | | AVQPRNVSYDSVSAIVTANLSIPSNWTTSVQVEYLQITSTPIVVDCSTYVCNGNVRCVELLKQYTSACK | |
| | | | TIEDALRNSAMLESADVSEMLTFDKKAFTLANVSSFGDYNLSSVIPSLPRSGSRVAGRSAIEDILFSKLV | |
| | | | TSGLGTVDADYKKCTKGLSIADLACAQYYNGIMVLPGVADAERMAMYTGSLIGGIALGGLTSAASIPFS | |
| | | | LAIQSRLNYVALQTDVLQENQKILAASFNKAMTNIVDAFTGVNDAITQTSQALQTVATALNKIQDVVNQQ | |
| | | | GNSLNHLTSQLRQNFQAISSSIQAIYDRLDPPQADQQVDRLITGRLAALNVFVSHTLTKYTEVRASRQL | |
| | | | AQQKVNECVKSQSKRYGFCGNGTHIFSLVNAAPEGLVFLHTVLLPTQYKDVEAWSGLCVDGRNGYVL | |
| | | | RQPNLALYKEGNYYRITSRIMFEPRIPTIADFVQIENCNVTFVNISRSELQTIVPEYIDLNKTLQELSYKLP | |
| | | | NLTVKGSGGSGDIIKLLNEQVNKEMQSSNLYMSMSSWCYTHSLDGAGLFLFDHAAEEYEHAKKLIIFLN | |
| | | | ENNVPVQLTSISAPEHKFEGLTQIFQKAYEHEQHISESINNIVDHAIKSKDHATFNFLQWYVAEQHEEEV | |
| | | | LFKDILDKIELIGNENHGLYLADQYVKGIAKSRKSGS | |
| p229E-F02 | mosaic SpFN | 229E SpFN with NL63 | CQTTNGLNTSYSVCNGCVGYSENVFAVESGGYIPSDFAFNNWFLLTNTSSVVDGVVRSFQPLLLNCL | 136 |
| | | RBD-SD1 replacement | WSVSGLRFTTGFVYFNGTGRGDCKGFSSDVLSDVIRYNLNFEENLRRGTILFKTSYGVVVFYCTNNTL | |
| | | | VSGDAHIPFGTVLGNFYCFVNTTIGNETTSAFVGALPKTVREFVISRTGHFYINGYRYFTLGNVEAVNF | |
| | | | NVTTAETTDFCTVALASYADVLVNVSQTSIANIIYCNSVINRLRCDQLSFDVPDGFYSTSPIQSVELPGS | |
| | | | ETYVALPIYYQHTDINFTATASFGGSCYVCKPHQVNISLNGNTSVCVRTSHFSIRYIYNRVKSGSPGDS | |
| | | | SWHIYLKSGTCPFSFSKLNNFQKFKTICFSTVEVPGSCNFPLEATWHYTSYTIVGALYVTWSEGNSITG | |
| | | | VPYPVSGIREFSNLVLNNCTKYNIYDYVGTGIIRSSNQSLAGGITYVSNSGNLLGFKNVSTGNIFIVTPCN | |
| | | | GSPPDQLVVYQQAVVGAMLSENFTSYGFSNVVELPKFFYASNGTYNCTDAVLTYSSFGVCADGSIIAV | |
| | | | QPRNVSYDSVSAIVTANLSIPSNWTTSVQVEYLQITSTPIWVDCSTYVCNGNVRCVELLKQYTSACKTIE | |
| | | | DALRNSAMLESADVSEMLTFDKKAFTLANVSSFGDYNLSSVIPSLPRSGSRVAGRSAIEDILFSKLVTS | |
| | | | GLGTVDADYKKCTKGLSIADLACAQYYNGIMVLPGVADAERMAMYTGSLIGGIALGGLTSAASIPFSLAI | |
| | | | QSRLNYVALQTDVLQENQKILAASFNKAMTNIVDAFTGVNDAITQTSQALQTVATALNKIQDVVNQQGN | |
| | | | SLNHLTSQLRQNFQAISSSIQAIYDRLDPPQADQQVDRLITGRLAALNVFVSHTLTKYTEVRASRQLAQ | |
| | | | QKVNECVKSQSKRYGFCGNGTHIFSLVNAAPEGLVFLHTVLLPTQYKDVEAWSGLCVDGRNGYVLRQ | |
| | | | PNLALYKEGNYYRITSRIMFEPRIPTIADFVQIENCNVTFVNISRSELQTIVPEYIDLNKTLQELSYKLPNLT | |
| | | | VKGSGGSGDIIKLLNEQVNKEMQSSNLYMSMSSWCYTHSLDGAGLFLFDHAAEEYEHAKKLIIFLNEN | |
| | | | NVPVQLTSISAPEHKFEGLTQIFQKAYEHEQHISESINNIVDHAIKSKDHATFNFLQWYVAEQHEEEVLF | |
| | | | KDILDKIELIGNENHGLYLADQYVKGIAKSRKSGS | |
| pCoV266 | SpFN | BA.1 Omicron SpFN | SSQCVNLTTRTQLPPAYTNSFTRGVYYPDKVFRSSVLHSTQDLFLPFFSNVTWFHVISGTNGTKRFDN | 137 |
| | | (1B-06-PL) | PVLPFNDGVYFASIEKSNIIRGWIFGTTLDSKTQSLLIVNNATNVVIKVCEFQFCNDPFLDHKNNKSWME | |
| | | | SEFRVYSSANNCTFEYVSQPFLMDLEGKQGNFKNLREFVFKNIDGYFKIYSKHTPIIVREPEDLPQGFS | |
| | | | ALEPLVDLPIGINITRFQTLLALHRSYLTPGDSSSGWTAGAAAYYVGYLQPRTFLLKYNENGTITDAVDC | |
| | | | ALDPLSETKCTLKSFTVEKGIYQTSNFRVQPTESIVRFPNITNLCPFDEVFNATRFASVYAWNRKRISNC | |
| | | | VADYSVLYNLAPFFTFKCYGVSPTKLNDLCFTNVYADSFVIRGDEVRQIAPGQTGNIADYNYKLPDDFT | |
| | | | GCVIAWNSNKLDSKVSGNYNYLYRLFRKSNLKPFERDISTEIYQAGNKPCNGVAGFNCYFPLRSYSFR | |
| | | | PTYGVGHQPYRVVVLSFELLHAPATVCGPKKSTNLVKNKCVNFNFNGLKGTGVLTESNKKFLPFQQF | |
| | | | GRDIADTTDAVRDPQTLEILDITPCSFGGVSVITPGTNTSNQVAVLYQGVNCTEVPVAIHADQLTPTWR | |
| | | | VYSTGSNVFQTRAGCLIGAEYVNNSYECDIPIGAGICASYQTQTKSHGSASSVASQSIIAYTMSLGAEN | |
| | | | SVAYSNNSIAIPTNFTISVTTEILPVSMTKTSVDCTMYICGDSTECSNLLLQYGSFCTQLKRALTGIAVEQ | |
| | | | DKNTQEVFAQVKQIYKTPPIKYFGGFNFSQILPDPSKPSKRSFIEDLLFNKVTLADAGFIKQYGDCLGDI | |
| | | | AARDLICAQKFKGLTVLPPLLTDEMIAQYTSALLAGTITSGWTFGAGAALQIPFAMQMAYRFNGIGVTQ | |
| | | | NVLYENQKLIANQFNSAIGKIQDSLSSTASALGKLQDVVNHNAQALNTLVKQLSSKFGAISSVLNDIFSR | |
| | | | LDPPEAEVQIDRLITGRLQSLQTYVTQQLIRAAEIRASANLAATKMSECVLGQSKRVDFCGKGYHLMSF | |
| | | | PQSAPHGVVFLHVTYVPAQEKNFTTAPAICHDGKAHFPREGVFVSNGTHWFVTQRNFYEPQIITTDNT | |
| | | | FVSGNCDVVIGIVNNTVYDPLQSELDSIKEELDKIHKNGSGGSGDIIKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| pCoVP501 | R-SpFN | RBD from Omicron | NITNLCPFDEVFNATRFASVYAWNRKRISNCVADYSVLYNFAPFFAFKCYGVSPTKLNDLCFTNVYADS | 138 |
| | | BA.4/5_SpFN | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP502 | R-SpFN | RBD from Omicron | NITNLCPFDEVFNATRFASVYAWNRKRISNCVADYSVLYNFAPFFAFKCYGVSPTKLNDLCFTNVYADS | 139 |
| | | BA.2.12.1_SpFN | FVIRGNEVSQIAPGQTGNIADYNYKLPDDFTGCVIAWNSNKLDSKVGGNYNYQYRLFRKSNLKPFERD | |
| | | | ISTEIYQAGNKPCNGVAGFNCYFPLRSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP503 | R-SpFN | RBD from NC_014470.BM4831. | NITQLCPFNEVFNITSFPSVYAWERMRITNCVADYSVLYNSSASFSTFQCYGVSPTKLNDLCFSSVYAD | 140 |
| | | BGR.2008_SpFN | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLASYGFTQSSGIGFQPYRVVVLSFELLNAPATVCGPGSGGSGSSQCV | |
| | | | NLTTRTQLPPAYTNSFTRGVYYPDKVFRSSVLHSTQDLFLPFFSNVTWFHAIHVSGTNGTKRFDNPVL | |
| | | | PFNDGVYFASTEKSNIIRGWIFGTTLDSKTQSLLIVNNATNVVIKVCEFQFCNDPFLGVYYHKNNKSWM | |
| | | | ESEFRVYSSANNCTFEYVSQPFLMDLEGKQGNFKNLREFVFKNIDGYFKIYSKHTPINLVRDLPQGFSA | |
| | | | LEPLVDLPIGINITRFQTLLALHRSYLTPGDSSSGWTAGAAAYYVGYLQPRTFLLKYNENGTITDAVDCA | |
| | | | LDPLSETKCTLKSFTVEKGIYQTSNFRVQPTESIVRFPNITNLCPFGEVFNATRFASVYAWNRKRISNC | |
| | | | VADYSVLYNSASFSTFKCYGVSPTKLNDLCFTNVYADSFVIRGDEVRQIAPGQTGKIADYNYKLPDDFT | |
| | | | GCVIAWNSNNLDSKVGGNYNYLYRLFRKSNLKPFERDISTEIYQAGSTPCNGVEGFNCYFPLQSYGFQ | |
| | | | PTNGVGYQPYRVVVLSFELLHAPATVCGPKKSTNLVKNKCVNFNFNGLTGTGVLTESNKKFLPFQQF | |
| | | | GRDIADTTDAVRDPQTLEILDITPCSFGGVSVITPGTNTSNQVAVLYQDVNCTEVPVAIHADQLTPTWR | |
| | | | VYSTGSNVFQTRAGCLIGAEHVNNSYECDIPIGAGICASYQTQTNSPGSASSVASQSIIAYTMSLGAEN | |
| | | | SVAYSNNSIAIPTNFTISVTTEILPVSMTKTSVDCTMYICGDSTECSNLLLQYGSFCTQLNRALTGIAVEQ | |
| | | | DKNTQEVFAQVKQIYKTPPIKDFGGFNFSQILPDPSKPSKRSFIEDLLFNKVTLADAGFIKQYGDCLGDI | |
| | | | AARDLICAQKFNGLTVLPPLLTDEMIAQYTSALLAGTITSGWTFGAGAALQIPFAMQMAYRENGIGVTQ | |
| | | | NVLYENQKLIANQFNSAIGKIQDSLSSTASALGKLQDVVNQNAQALNTLVKQLSSNFGAISSVLNDILSR | |
| | | | LDPPEAEVQIDRLITGRLQSLQTYVTQQLIRAAEIRASANLAATKMSECVLGQSKRVDFCGKGYHLMSF | |
| | | | PQSAPHGVVFLHVTYVPAQEKNFTTAPAICHDGKAHFPREGVFVSNGTHWFVTQRNFYEPQIITTDNT | |
| | | | FVSGNCDVVIGIVNNTVYDPLQSELDSIKEELDKIHKNGSGGSGDIIKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| pCoVP504 | R-SpFN | BM48-31 RBD-T498W_SpFN | NITQLCPFNEVFNITSFPSVYAWERMRITNCVADYSVLYNSSASFSTFQCYGVSPTKLNDLCFSSVYAD | 141 |
| | | | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLASYGFWQSSGIGFQPYRVVVLSFELLNAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATRFASVYAWNRKRIS | |
| | | | NCVADYSVLYNSASFSTFKCYGVSPTKLNDLCFTNVYADSFVIRGDEVRQIAPGQTGKIADYNYKLPDD | |
| | | | FTGCVIAWNSNNLDSKVGGNYNYLYRLFRKSNLKPFERDISTEIYQAGSTPCNGVEGFNCYFPLQSYG | |
| | | | FQPTNGVGYQPYRVVVLSFELLHAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP505 | R-SpFN | BM48-31 RBD-A493Y- | NITQLCPFNEVFNITSFPSVYAWERMRITNCVADYSVLYNSSASFSTFQCYGVSPTKLNDLCFSSVYAD | 142 |
| | | T498W_SpFN | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLYSYGFWQSSGIGFQPYRVVVLSFELLNAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATRFASVYAWNRKRIS | |
| | | | NCVADYSVLYNSASFSTFKCYGVSPTKLNDLCFTNVYADSFVIRGDEVRQIAPGQTGKIADYNYKLPDD | |
| | | | FTGCVIAWNSNNLDSKVGGNYNYLYRLFRKSNLKPFERDISTEIYQAGSTPCNGVEGFNCYFPLQSYG | |
| | | | FQPTNGVGYQPYRVVVLSFELLHAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP506 | R-SpFN | RBD from KC881005. | NITNLCPFGEVFNATTFPSVYAWERKRISNCVADYSVLYNSTSFSTFKCYGVSATKLNDLCFSNVYADS | 143 |
| | | RsSHC014_SpFN | FVVKGDDVRQIAPGQTGVIADYNYKLPDDFLGCVLAWNTNSKDSSTSGNYNYLYRWVRRSKLNPYER | |
| | | | DLSNDIYSPGGQSCSAVGPNCYNPLRPYGFFTTAGVGHQPYRVVVLSFELLNAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP507 | R-SpFN | RBD from KC881007.Bat. | NITNLCPFGEVFNATTFPSVYAWERKRISNCVADYSVLYNSTSFSTFKCYGVSATKLNDLCFSNVYADS | 144 |
| | | WIV1_SpFN | FVVKGDDVRQIAPGQTGVIADYNYKLPDDFTGCVLAWNTRNIDATQTGNYNYKYRSLRHGKLRPFER | |
| | | | DISNVPFSPDGKPCTPPAFNCYWPLNDYGFYITNGIGYQPYRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP508 | R-SpFN | RBD from AVP78042_ | NITNVCPFHKVFNATRFPSVYAWERTKISDCIADYTVFYNSTSFSTFKCYGVSPSKLIDLCFTSVYADTF | 145 |
| | | BatSARSlikecoronavirus_ | LIRFSEVRQVAPGQTGVIADYNYKLPDDFTGCVIAWNTAKQDTGHYFYRSHRSTKLKPFERDLSSDEN | |
| | | SpFN | GVRTLSTYDFNPNVPLEYQATRVVVLSFELLNAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP509 | R-SpFN | RBD from MZ937001_ | NITNLCPFGEVENATTFASVYAWNRKRISNCVADYSVLYNSTSFSTFKCYGVSPTKLNDLCFTNVYADS | 146 |
| | | BANAL20103_Laos_2020_SpFN | FVVRGDEVRQIAPGQTGKIADYNYKLPDDFTGCVIAWNSNNLDSKVGGNYNYLYRLFRKSNLKPFERD | |
| | | | ISTEIYQAGSTPCNGVEGFNCYFPLQSYGFHPTNGVGYQPYRVVVLSFELLNAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGFNCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP510 | R-SpFN | RBD from MN996532.2013. | NITNLCPFGEVFNATTFASVYAWNRKRISNCVADYSVLYNSTSFSTFKCYGVSPTKLNDLCFTNVYADS | 147 |
| | | Bat.RaTG13_SpFN | FVITGDEVRQIAPGQTGKIADYNYKLPDDFTGCVIAWNSKHIDAKEGGNFNYLYRLFRKANLKPFERDI | |
| | | | STEIYQAGSKPCNGQTGLNCYYPLYRYGFYPTDGVGHQPYRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP511 | R-SpFN | RBD from MZ937004_ | NITNFCPFDKVFNATRFPNVYAWQRTKISDCIADYTVLYNSTSFSTFKCYGVSPSKLIDLCFTSVYADTF | 148 |
| | | BANAL20247_Laos_2020_SpFN | LIRFSEVRQIAPGETGVIADYNYKLPDDFTGCVLAWNTAQQDIGSYFYRSHRAVKLKPFERDLSSDEN | |
| | | | GVRTLSTYDFNPNVPLDYQATRVVVLSFELLNAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRENGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP512 | R-SpFN | BANAL-20-247 | NITNFCPFDKVFNATRFPNVYAWQRTKISDCIADYTVLYNSTSFSTFKCYGVSPSKLIDLCFTSVYADTF | 149 |
| | | RBD-N498W_SpFN | LIRFSEVRQIAPGETGVIADYNYKLPDDFTGCVLAWNTAQQDIGSYFYRSHRAVKLKPFERDLSSDEN | |
| | | | GVRTLSTYDFWPNVPLDYQATRVVVLSFELLNAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP513 | R-SpFN | BANAL-20-247 | NITNFCPFDKVFNATRFPNVYAWQRTKISDCIADYTVLYNSTSFSTFKCYGVSPSKLIDLCFTSVYADTF | 150 |
| | | RBD-S493Y-N498W_SpFN | LIRFSEVRQIAPGETGVIADYNYKLPDDFTGCVLAWNTAQQDIGSYFYRSHRAVKLKPFERDLSSDEN | |
| | | | GVRTLYTYDFWPNVPLDYQATRVVVLSFELLNAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP514 | R-SpFN | RBD from ATO98145_ | NITNLCPFDAVFNATRFPSVYAWERVKISNCVADYTAFYNSTSFSTFKCYGVSPSKLIDLCFTSVYADT | 151 |
| | | BatSARSlikecoronavirus_ | FLIRFSEVRQVAPGETGVIADYNYKLPDDFTGCVIAWNTAKQDVGSYFYRSHRSSKLKPFERDLSSDE | |
| | | SpFN | NGVRTLSTYDFNPNVPLDYQATRVVVLSFELLNAPATVCGPGSGGSGSSQCVNLTTRTQLPPAYTNS | |
| | | | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATRFASVYAWNRKRISNCVADYSVLYNSASFSTF | |
| | | | KCYGVSPTKLNDLCFTNVYADSFVIRGDEVRQIAPGQTGKIADYNYKLPDDFTGCVIAWNSNNLDSKV | |
| | | | GGNYNYLYRLFRKSNLKPFERDISTEIYQAGSTPCNGVEGFNCYFPLQSYGFQPTNGVGYQPYRVVV | |
| | | | LSFELLHAPATVCGPKKSTNLVKNKCVNFNFNGLTGTGVLTESNKKFLPFQQFGRDIADTTDAVRDPQ | |
| | | | TLEILDITPCSFGGVSVITPGTNTSNQVAVLYQDVNCTEVPVAIHADQLTPTWRVYSTGSNVFQTRAGC | |
| | | | LIGAEHVNNSYECDIPIGAGICASYQTQTNSPGSASSVASQSIIAYTMSLGAENSVAYSNNSIAIPTNFTI | |
| | | | SVTTEILPVSMTKTSVDCTMYICGDSTECSNLLLQYGSFCTQLNRALTGIAVEQDKNTQEVFAQVKQIY | |
| | | | KTPPIKDFGGFNFSQILPDPSKPSKRSFIEDLLFNKVTLADAGFIKQYGDCLGDIAARDLICAQKFNGLTV | |
| | | | LPPLLTDEMIAQYTSALLAGTITSGWTFGAGAALQIPFAMQMAYRFNGIGVTQNVLYENQKLIANQFNS | |
| | | | AIGKIQDSLSSTASALGKLQDVVNQNAQALNTLVKQLSSNFGAISSVLNDILSRLDPPEAEVQIDRLITGR | |
| | | | LQSLQTYVTQQLIRAAEIRASANLAATKMSECVLGQSKRVDFCGKGYHLMSFPQSAPHGVVFLHVTYV | |
| | | | PAQEKNFTTAPAICHDGKAHFPREGVFVSNGTHWFVTQRNFYEPQIITTDNTFVSGNCDWVIGIVNNTV | |
| | | | YDPLQSELDSIKEELDKIHKNGSGGSGDIIKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP515 | R-SpFN | RBD from JX993987.2011. | NITNRCPFDKVFNATRFPSVYAWERTKISDCVADYTVLYNSTSFSTFKCYGVSPSKLIDLCFTSVYADT | 152 |
| | | BatRp.Shaanxi2011_SpFN | FLIRSSEVRQVAPGETGVIADYNYKLPDDFTGCVIAWNTANQDQGQYYYRSSRKEKLKPFERDLSSDE | |
| | | | NGVYTLSTYDFYPSVPLDYQATRVVVLSFELLNAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP516 | R-SpFN | RBD from KJ473812. | NITNLCPFDKVFNATRFPSVYAWERTKISDCVADYTVFYNSTSFSTFNCYGVSPSKLIDLCFTSVYADT | 153 |
| | | BtRfBetaCoV.HeB2013_SpFN | FLIRFSEVRQVAPGQTGVIADYNYKLPDDFTGCVIAWNTAKQDVGSYFYRSHRSSKLKPFERDLSSEE | |
| | | | NGVRTLSTYDFNQYVPLEYQATRVVVLSFELLNAPATVCGPGSGGSGSSQCVNLTTRTQLPPAYTNS | |
| | | | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATRFASVYAWNRKRISNCVADYSVLYNSASFSTF | |
| | | | KCYGVSPTKLNDLCFTNVYADSFVIRGDEVRQIAPGQTGKIADYNYKLPDDFTGCVIAWNSNNLDSKV | |
| | | | GGNYNYLYRLFRKSNLKPFERDISTEIYQAGSTPCNGVEGFNCYFPLQSYGFQPTNGVGYQPYRVVV | |
| | | | LSFELLHAPATVCGPKKSTNLVKNKCVNFNFNGLTGTGVLTESNKKFLPFQQFGRDIADTTDAVRDPQ | |
| | | | TLEILDITPCSFGGVSVITPGTNTSNQVAVLYQDVNCTEVPVAIHADQLTPTWRVYSTGSNVFQTRAGC | |
| | | | LIGAEHVNNSYECDIPIGAGICASYQTQTNSPGSASSVASQSIIAYTMSLGAENSVAYSNNSIAIPTNFTI | |
| | | | SVTTEILPVSMTKTSVDCTMYICGDSTECSNLLLQYGSFCTQLNRALTGIAVEQDKNTQEVFAQVKQIY | |
| | | | KTPPIKDFGGFNFSQILPDPSKPSKRSFIEDLLFNKVTLADAGFIKQYGDCLGDIAARDLICAQKFNGLTV | |
| | | | LPPLLTDEMIAQYTSALLAGTITSGWTFGAGAALQIPFAMQMAYRFNGIGVTQNVLYENQKLIANQFNS | |
| | | | AIGKIQDSLSSTASALGKLQDVVNQNAQALNTLVKQLSSNFGAISSVLNDILSRLDPPEAEVQIDRLITGR | |
| | | | LQSLQTYVTQQLIRAAEIRASANLAATKMSECVLGQSKRVDFCGKGYHLMSFPQSAPHGVVFLHVTYV | |
| | | | PAQEKNFTTAPAICHDGKAHFPREGVFVSNGTHWFVTQRNFYEPQIITTDNTFVSGNCDWVIGIVNNTV | |
| | | | YDPLQSELDSIKEELDKIHKNGSGGSGDIIKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP517 | R-SpFN | RBD from | NITNRCPFDKVFNATRFPNVYAWERTKISDCVADYTVLYNSTSFSTFKCYGVSPSKLIDLCFTSVYADT | 154 |
| | | DQ071615.2003.Bat.Rp3_SpFN | FLIRSSEVRQVAPGETGVIADYNYKLPDDFTGCVIAWNTAKQDQGQYYYRSHRKTKLKPFERDLSSDE | |
| | | | NGVRTLSTYDFYPSVPVAYQATRVVVLSFELLNAPATVCGPGSGGSGSSQCVNLTTRTQLPPAYTNS | |
| | | | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATRFASVYAWNRKRISNCVADYSVLYNSASFSTF | |
| | | | KCYGVSPTKLNDLCFTNVYADSFVIRGDEVRQIAPGQTGKIADYNYKLPDDFTGCVIAWNSNNLDSKV | |
| | | | GGNYNYLYRLFRKSNLKPFERDISTEIYQAGSTPCNGVEGFNCYFPLQSYGFQPTNGVGYQPYRVVV | |
| | | | LSFELLHAPATVCGPKKSTNLVKNKCVNFNFNGLTGTGVLTESNKKFLPFQQFGRDIADTTDAVRDPQ | |
| | | | TLEILDITPCSFGGVSVITPGTNTSNQVAVLYQDVNCTEVPVAIHADQLTPTWRVYSTGSNVFQTRAGC | |
| | | | LIGAEHVNNSYECDIPIGAGICASYQTQTNSPGSASSVASQSIIAYTMSLGAENSVAYSNNSIAIPTNFTI | |
| | | | SVTTEILPVSMTKTSVDCTMYICGDSTECSNLLLQYGSFCTQLNRALTGIAVEQDKNTQEVFAQVKQIY | |
| | | | KTPPIKDFGGFNFSQILPDPSKPSKRSFIEDLLFNKVTLADAGFIKQYGDCLGDIAARDLICAQKFNGLTV | |
| | | | LPPLLTDEMIAQYTSALLAGTITSGWTFGAGAALQIPFAMQMAYRFNGIGVTQNVLYENQKLIANQFNS | |
| | | | AIGKIQDSLSSTASALGKLQDVVNQNAQALNTLVKQLSSNFGAISSVLNDILSRLDPPEAEVQIDRLITGR | |
| | | | LQSLQTYVTQQLIRAAEIRASANLAATKMSECVLGQSKRVDFCGKGYHLMSFPQSAPHGVVFLHVTYV | |
| | | | PAQEKNFTTAPAICHDGKAHFPREGVFVSNGTHWFVTQRNFYEPQIITTDNTFVSGNCDWVIGIVNNTV | |
| | | | YDPLQSELDSIKEELDKIHKNGSGGSGDIIKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP518 | R-SpFN | RBD from | NITNRCPFDKVFNASRFPNVYAWERTKISDCVADYTVLYNSTSFSTFKCYGVSPSKLIDLCFTSVYADT | 155 |
| | | ACU31032_Rs_672_2006_SpFN | FLIRSSEVRQVAPGETGVIADYNYKLPDDFTGCVIAWNTAKQDQGQYYYRSSRKTKLKPFERDLTSDE | |
| | | | NGVRTLSTYDFYPNVPIEYQATRVVVLSFELLNAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRENGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP519 | R-SpFN | RBD from MZ081380_bat_ | NITNLCPFSQVFNATRFPSVYAWTRERISNCIADYSVLYNSTSFSTFRCYGVSPTKLNDLCFSNVYADS | 156 |
| | | Yunnan_RsYN04_2020_SpFN | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFTSSVSQDYQPYRVVVLSFELLNAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP520 | R-SpFN | RsYN04 RBD-T498W_SpFN | NITNLCPFSQVFNATRFPSVYAWTRERISNCIADYSVLYNSTSFSTFRCYGVSPTKLNDLCFSNVYADS | 157 |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFWSSVSQDYQPYRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP521 | R-SpFN | RsYN04 RBD-L493Y-T498W_ | NITNLCPFSQVFNATRFPSVYAWTRERISNCIADYSVLYNSTSFSTFRCYGVSPTKLNDLCFSNVYADS | 158 |
| | | SpFN | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLYKYGFWSSVSQDYQPYRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP522 | R-SpFN | Khosta2-MZ190138_SpFN | NMTNICPFDQVFNKTQFPSVYAWERVRISDCVSDYTVLYNSSASFSTFKCYGVSPTKLNDLCFSGVYA | 159 |
| | | | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFTSTSGVGYQPFRVVVLSFELLNAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP523 | R-SpFN | Khosta-2 RBD-T498W_SpFN | NMTNICPFDQVFNKTQFPSVYAWERVRISDCVSDYTVLYNSSASFSTFKCYGVSPTKLNDLCFSGVYA | 160 |
| | | | DYFWKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFWSTSGVGYQPFRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP524 | R-SpFN | Khosta-2 RBD-Q493Y- | NMTNICPFDQVFNKTQFPSVYAWERVRISDCVSDYTVLYNSSASFSTFKCYGVSPTKLNDLCFSGVYA | 161 |
| | | T498W_SpFN | DYFWKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLYDYGFWSTSGVGYQPFRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP525 | R-SpFN | KY352407_BtKY72_bat_ | NITNLCPFGQVFNASNFPSVYAWERLRISDCVADYAVLYNSSSSFSTFKCYGVSPTKLNDLCFSSVYA | 162 |
| | | Kenya_SpFN | DYFWKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFTPTVGVGYQPYRVVVLSFELLNAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP526 | R-SpFN | BtKY72 RBD-T498W _SpFN | NITNLCPFGQVFNASNFPSVYAWERLRISDCVADYAVLYNSSSSFSTFKCYGVSPTKLNDLCFSSVYA | 163 |
| | | | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFWPTVGVGYQPYRVVVLSFELLNAPATVCGPGSGGSG | |
| | | | SSQCVNLTTRTQLPPAYTNSFTRGVYYPDKVFRSSVLHSTQDLFLPFFSNVTWFHAIHVSGTNGTKRF | |
| | | | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATRFASVYAWNRK | |
| | | | RISNCVADYSVLYNSASFSTFKCYGVSPTKLNDLCFTNVYADSFVIRGDEVRQIAPGQTGKIADYNYKL | |
| | | | PDDFTGCVIAWNSNNLDSKVGGNYNYLYRLFRKSNLKPFERDISTEIYQAGSTPCNGVEGFNCYFPLQ | |
| | | | SYGFQPTNGVGYQPYRVVVLSFELLHAPATVCGPKKSTNLVKNKCVNFNFNGLTGTGVLTESNKKFL | |
| | | | PFQQFGRDIADTTDAVRDPQTLEILDITPCSFGGVSVITPGTNTSNQVAVLYQDVNCTEVPVAIHADQL | |
| | | | TPTWRVYSTGSNVFQTRAGCLIGAEHVNNSYECDIPIGAGICASYQTQTNSPGSASSVASQSIIAYTMS | |
| | | | LGAENSVAYSNNSIAIPTNFTISVTTEILPVSMTKTSVDCTMYICGDSTECSNLLLQYGSFCTQLNRALT | |
| | | | GIAVEQDKNTQEVFAQVKQIYKTPPIKDFGGFNFSQILPDPSKPSKRSFIEDLLFNKVTLADAGFIKQYG | |
| | | | DCLGDIAARDLICAQKFNGLTVLPPLLTDEMIAQYTSALLAGTITSGWTFGAGAALQIPFAMQMAYRFN | |
| | | | GIGVTQNVLYENQKLIANQFNSAIGKIQDSLSSTASALGKLQDVVNQNAQALNTLVKQLSSNFGAISSVL | |
| | | | NDILSRLDPPEAEVQIDRLITGRLQSLQTYVTQQLIRAAEIRASANLAATKMSECVLGQSKRVDFCGKG | |
| | | | YHLMSFPQSAPHGVVFLHVTYVPAQEKNFTTAPAICHDGKAHFPREGVFVSNGTHWFVTQRNFYEPQ | |
| | | | IITTDNTFVSGNCDVVIGIVNNTVYDPLQSELDSIKEELDKIHKNGSGGSGDIIKLLNEQVNKEMQSSNLY | |
| | | | MSMSSWCYTHSLDGAGLFLFDHAAEEYEHAKKLIIFLNENNVPVQLTSISAPEHKFEGLTQIFQKAYEH | |
| | | | EQHISESINNIVDHAIKSKDHATFNFLQWYVAEQHEEEVLFKDILDKIELIGNENHGLYLADQYVKGIAKS | |
| | | | RKSGS | |
| pCoVP527 | R-SpFN | BtKY72 RBD-K493Y-T498W_ | NITNLCPFGQVFNASNFPSVYAWERLRISDCVADYAVLYNSSSSFSTFKCYGVSPTKLNDLCFSSVYA | 164 |
| | | SpFN | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLYSYGFWPTVGVGYQPYRVVVLSFELLNAPATVCGPGSGGSG | |
| | | | SSQCVNLTTRTQLPPAYTNSFTRGVYYPDKVFRSSVLHSTQDLFLPFFSNVTWFHAIHVSGTNGTKRF | |
| | | | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATRFASVYAWNRK | |
| | | | RISNCVADYSVLYNSASFSTFKCYGVSPTKLNDLCFTNVYADSFVIRGDEVRQIAPGQTGKIADYNYKL | |
| | | | PDDFTGCVIAWNSNNLDSKVGGNYNYLYRLFRKSNLKPFERDISTEIYQAGSTPCNGVEGFNCYFPLQ | |
| | | | SYGFQPTNGVGYQPYRVVVLSFELLHAPATVCGPKKSTNLVKNKCVNFNFNGLTGTGVLTESNKKFL | |
| | | | PFQQFGRDIADTTDAVRDPQTLEILDITPCSFGGVSVITPGTNTSNQVAVLYQDVNCTEVPVAIHADQL | |
| | | | TPTWRVYSTGSNVFQTRAGCLIGAEHVNNSYECDIPIGAGICASYQTQTNSPGSASSVASQSIIAYTMS | |
| | | | LGAENSVAYSNNSIAIPTNFTISVTTEILPVSMTKTSVDCTMYICGDSTECSNLLLQYGSFCTQLNRALT | |
| | | | GIAVEQDKNTQEVFAQVKQIYKTPPIKDFGGFNFSQILPDPSKPSKRSFIEDLLFNKVTLADAGFIKQYG | |
| | | | DCLGDIAARDLICAQKFNGLTVLPPLLTDEMIAQYTSALLAGTITSGWTFGAGAALQIPFAMQMAYRFN | |
| | | | GIGVTQNVLYENQKLIANQFNSAIGKIQDSLSSTASALGKLQDVVNQNAQALNTLVKQLSSNFGAISSVL | |
| | | | NDILSRLDPPEAEVQIDRLITGRLQSLQTYVTQQLIRAAEIRASANLAATKMSECVLGQSKRVDFCGKG | |
| | | | YHLMSFPQSAPHGVVFLHVTYVPAQEKNFTTAPAICHDGKAHFPREGVFVSNGTHWFVTQRNFYEPQ | |
| | | | IITTDNTFVSGNCDVVIGIVNNTVYDPLQSELDSIKEELDKIHKNGSGGSGDIIKLLNEQVNKEMQSSNLY | |
| | | | MSMSSWCYTHSLDGAGLFLFDHAAEEYEHAKKLIIFLNENNVPVQLTSISAPEHKFEGLTQIFQKAYEH | |
| | | | EQHISESINNIVDHAIKSKDHATFNFLQWYVAEQHEEEVLFKDILDKIELIGNENHGLYLADQYVKGIAKS | |
| | | | RKSGS | |
| pCoVP528 | R-SpFN | RaTG15_Ra7909 (RBD)_SpFN | NITNLCPFSQVFNATRFPSVYAWTRERISNCIADYSVLYNSTSFSTFRCYGVSPTKLNDLCFSNVYADS | 165 |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFTGSVSQDYQPYRVVVLSFELLNAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGENCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP529 | R-SpFN | HKU3-1_DQ022305.2005 | NITNRCPFDKVFNATRFPNVYAWERTKISDCVADYTVLYNSTSFSTFKCYGVSPSKLIDLCFTSVYADT | 166 |
| | | (RBD)_SpFN | FLIRSSEVRQVAPGETGVIADYNYKLPDDFTGCVIAWNTAKHDTGNYYYRSHRKTKLKPFERDLSSDD | |
| | | | GNGVYTLSTYDFNPNVPVAYQATRVVVLSFELLNAPATVCGPGSGGSGSSQCVNLTTRTQLPPAYTN | |
| | | | SFTRGVYYPDKVFRSSVLHSTQDLFLPFFSNVTWFHAIHVSGTNGTKRFDNPVLPFNDGVYFASTEKS | |
| | | | NIIRGWIFGTTLDSKTQSLLIVNNATNVVIKVCEFQFCNDPFLGVYYHKNNKSWMESEFRVYSSANNCT | |
| | | | FEYVSQPFLMDLEGKQGNFKNLREFVFKNIDGYFKIYSKHTPINLVRDLPQGFSALEPLVDLPIGINITRF | |
| | | | QTLLALHRSYLTPGDSSSGWTAGAAAYYVGYLQPRTFLLKYNENGTITDAVDCALDPLSETKCTLKSF | |
| | | | TVEKGIYQTSNFRVQPTESIVRFPNITNLCPFGEVFNATRFASVYAWNRKRISNCVADYSVLYNSASFS | |
| | | | TFKCYGVSPTKLNDLCFTNVYADSFVIRGDEVRQIAPGQTGKIADYNYKLPDDFTGCVIAWNSNNLDS | |
| | | | KVGGNYNYLYRLFRKSNLKPFERDISTEIYQAGSTPCNGVEGFNCYFPLQSYGFQPTNGVGYQPYRV | |
| | | | WLSFELLHAPATVCGPKKSTNLVKNKCVNFNFNGLTGTGVLTESNKKFLPFQQFGRDIADTTDAVRD | |
| | | | PQTLEILDITPCSFGGVSVITPGTNTSNQVAVLYQDVNCTEVPVAIHADQLTPTWRVYSTGSNVFQTRA | |
| | | | GCLIGAEHVNNSYECDIPIGAGICASYQTQTNSPGSASSVASQSIIAYTMSLGAENSVAYSNNSIAIPTN | |
| | | | FTISVTTEILPVSMTKTSVDCTMYICGDSTECSNLLLQYGSFCTQLNRALTGIAVEQDKNTQEVFAQVK | |
| | | | QIYKTPPIKDFGGFNFSQILPDPSKPSKRSFIEDLLFNKVTLADAGFIKQYGDCLGDIAARDLICAQKENG | |
| | | | LTVLPPLLTDEMIAQYTSALLAGTITSGWTFGAGAALQIPFAMQMAYRFNGIGVTQNVLYENQKLIANQ | |
| | | | FNSAIGKIQDSLSSTASALGKLQDVVNQNAQALNTLVKQLSSNFGAISSVLNDILSRLDPPEAEVQIDRLI | |
| | | | TGRLQSLQTYVTQQLIRAAEIRASANLAATKMSECVLGQSKRVDFCGKGYHLMSFPQSAPHGVVFLH | |
| | | | VTYVPAQEKNFTTAPAICHDGKAHFPREGVFVSNGTHWFVTQRNFYEPQIITTDNTFVSGNCDVVIGIV | |
| | | | NNTVYDPLQSELDSIKEELDKIHKNGSGGSGDIIKLLNEQVNKEMQSSNLYMSMSSWCYTHSLDGAGL | |
| | | | FLFDHAAEEYEHAKKLIIFLNENNVPVQLTSISAPEHKFEGLTQIFQKAYEHEQHISESINNIVDHAIKSKD | |
| | | | HATFNFLQWYVAEQHEEEVLFKDILDKIELIGNENHGLYLADQYVKGIAKSRKSGS | |
| pCoVP530 | R-SpFN | RhGB01-MW719567 | NVTQLCPFSDVFNRTMFPSVYAWERMSIRDCVADYAVLYNSSISFSTFECYGVSPTKLNDLCFSSVYA | 167 |
| | | (RBD)_SpFN | DYFWVRGDDVKQIAPGKTGVIADYNYKLPDDFVGCVLAWNSRHVDNKQGFYYRLFRHGKIKPYQRDM | |
| | | | SRELYSPSGTPCTVVGFNCYDPLAHYAFASPGIGIGYQPFRVVVLSFQLLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVWVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP531 | R-SpFN | bioinformatically derived | NITNLCPFDEVFNATRFPSVYAWERKKISNCVADYSVLYNSTSFSTFKCYGVSPSKLNDLCFTSVYADS | 168 |
| | | Cluster1_RBD (SARS- | FVIRGDEVRQIAPGQTGVIADYNYKLPDDFTGCVIAWNTANQDSGEGGNPNYYYRSSRKSKLKPFER | |
| | | related)_SpFN | DLSSDEYNVGSQPCNRMGPNCYRTLSTYDFYPTVGVGYQPYRVVVLSFELLNAPATVCGPGSGGSG | |
| | | | SSQCVNLTTRTQLPPAYTNSFTRGVYYPDKVFRSSVLHSTQDLFLPFFSNVTWFHAIHVSGTNGTKRF | |
| | | | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATRFASVYAWNRK | |
| | | | RISNCVADYSVLYNSASFSTFKCYGVSPTKLNDLCFTNVYADSFVIRGDEVRQIAPGQTGKIADYNYKL | |
| | | | PDDFTGCVIAWNSNNLDSKVGGNYNYLYRLFRKSNLKPFERDISTEIYQAGSTPCNGVEGFNCYFPLQ | |
| | | | SYGFQPTNGVGYQPYRVVVLSFELLHAPATVCGPKKSTNLVKNKCVNFNFNGLTGTGVLTESNKKFL | |
| | | | PFQQFGRDIADTTDAVRDPQTLEILDITPCSFGGVSVITPGTNTSNQVAVLYQDVNCTEVPVAIHADQL | |
| | | | TPTWRVYSTGSNVFQTRAGCLIGAEHVNNSYECDIPIGAGICASYQTQTNSPGSASSVASQSIIAYTMS | |
| | | | LGAENSVAYSNNSIAIPTNFTISVTTEILPVSMTKTSVDCTMYICGDSTECSNLLLQYGSFCTQLNRALT | |
| | | | GIAVEQDKNTQEVFAQVKQIYKTPPIKDFGGFNFSQILPDPSKPSKRSFIEDLLFNKVTLADAGFIKQYG | |
| | | | DCLGDIAARDLICAQKFNGLTVLPPLLTDEMIAQYTSALLAGTITSGWTFGAGAALQIPFAMQMAYRFN | |
| | | | GIGVTQNVLYENQKLIANQFNSAIGKIQDSLSSTASALGKLQDVVNQNAQALNTLVKQLSSNFGAISSVL | |
| | | | NDILSRLDPPEAEVQIDRLITGRLQSLQTYVTQQLIRAAEIRASANLAATKMSECVLGQSKRVDFCGKG | |
| | | | YHLMSFPQSAPHGVVFLHVTYVPAQEKNFTTAPAICHDGKAHFPREGVFVSNGTHWFVTQRNFYEPQ | |
| | | | IITTDNTFVSGNCDVVIGIVNNTVYDPLQSELDSIKEELDKIHKNGSGGSGDIIKLLNEQVNKEMQSSNLY | |
| | | | MSMSSWCYTHSLDGAGLFLFDHAAEEYEHAKKLIIFLNENNVPVQLTSISAPEHKFEGLTQIFQKAYEH | |
| | | | EQHISESINNIVDHAIKSKDHATFNFLQWYVAEQHEEEVLFKDILDKIELIGNENHGLYLADQYVKGIAKS | |
| | | | RKSGS | |
| pCoVP532 | R-SpFN | bioinformatically derived | NLPDCNIEEWLTAKSVPSPLNWERKTFSNCNFNLSSLLRFVQADSFSCNNIDASKVYGMCFGSISVDK | 169 |
| | | Cluster2_RBD (OC43- | FAIPNSRQVDLQLGNSGFLQSFNYKIDTTATSCQLYYSLPANNVTINNYNPSSWNRRYGFNDDTVFKP | |
| | | related)_SpFN | RPAGVLTNHDVVYSQQCFKVPNTFCPCKNNGSCVSSGKGTTNGSGTCPAGTNYRTCENTTWCACS | |
| | | | CTPDPITASGPRKCPQKKSLVGVGEHCSGLGVDSDYCGNVSCLCSTDAFLGWSADSCLVNDRCNIFA | |
| | | | NIILNGINSGTTCSTGSGGSGSSQCVNLTTRTQLPPAYTNSFTRGVYYPDKVFRSSVLHSTQDLFLPFF | |
| | | | SNVTWFHAIHVSGTNGTKRFDNPVLPFNDGVYFASTEKSNIIRGWIFGTTLDSKTQSLLIVNNATNVVIK | |
| | | | VCEFQFCNDPFLGVYYHKNNKSWMESEFRVYSSANNCTFEYVSQPFLMDLEGKQGNFKNLREFVFK | |
| | | | NIDGYFKIYSKHTPINLVRDLPQGFSALEPLVDLPIGINITRFQTLLALHRSYLTPGDSSSGWTAGAAAYY | |
| | | | VGYLQPRTFLLKYNENGTITDAVDCALDPLSETKCTLKSFTVEKGIYQTSNFRVQPTESIVRFPNITNLC | |
| | | | PFGEVFNATRFASVYAWNRKRISNCVADYSVLYNSASFSTFKCYGVSPTKLNDLCFTNVYADSFVIRG | |
| | | | DEVRQIAPGQTGKIADYNYKLPDDFTGCVIAWNSNNLDSKVGGNYNYLYRLFRKSNLKPFERDISTEIY | |
| | | | QAGSTPCNGVEGFNCYFPLQSYGFQPTNGVGYQPYRVVVLSFELLHAPATVCGPKKSTNLVKNKCV | |
| | | | NFNFNGLTGTGVLTESNKKFLPFQQFGRDIADTTDAVRDPQTLEILDITPCSFGGVSVITPGTNTSNQV | |
| | | | AVLYQDVNCTEVPVAIHADQLTPTWRVYSTGSNVFQTRAGCLIGAEHVNNSYECDIPIGAGICASYQT | |
| | | | QTNSPGSASSVASQSIIAYTMSLGAENSVAYSNNSIAIPTNFTISVTTEILPVSMTKTSVDCTMYICGDST | |
| | | | ECSNLLLQYGSFCTQLNRALTGIAVEQDKNTQEVFAQVKQIYKTPPIKDFGGFNFSQILPDPSKPSKRS | |
| | | | FIEDLLFNKVTLADAGFIKQYGDCLGDIAARDLICAQKFNGLTVLPPLLTDEMIAQYTSALLAGTITSGWT | |
| | | | FGAGAALQIPFAMQMAYRFNGIGVTQNVLYENQKLIANQFNSAIGKIQDSLSSTASALGKLQDVVNQN | |
| | | | AQALNTLVKQLSSNFGAISSVLNDILSRLDPPEAEVQIDRLITGRLQSLQTYVTQQLIRAAEIRASANLAA | |
| | | | TKMSECVLGQSKRVDFCGKGYHLMSFPQSAPHGVVFLHVTYVPAQEKNFTTAPAICHDGKAHFPREG | |
| | | | VFVSNGTHWFVTQRNFYEPQIITTDNTFVSGNCDVVIGIVNNTVYDPLQSELDSIKEELDKIHKNGSGG | |
| | | | SGDIIKLLNEQVNKEMQSSNLYMSMSSWCYTHSLDGAGLFLFDHAAEEYEHAKKLIIFLNENNVPVQLT | |
| | | | SISAPEHKFEGLTQIFQKAYEHEQHISESINNIVDHAIKSKDHATFNFLQWYVAEQHEEEVLFKDILDKIE | |
| | | | LIGNENHGLYLADQYVKGIAKSRKSGS | |
| pCoVP533 | R-SpFN | bioinformatically derived | QGVECDFSKLFKAAAPQIYNFSRLVFTNCNYNLTKLLSLFQVDEFSCHQISPSALASGCYSSLTVDYFA | 170 |
| | | Cluster3_RBD (MERS- | YPLSMKSYLQIGSAGEISQFNYKQDFSNPTCRILASVPANVSITKPDKYIYISQCYSLSAYSDVDVPHYV | |
| | | related)_SpFN | NPGQYTSIPCLYLTSSGFDEDGQYFRYNLTQFEGQNYLVATGVTVSMTDNLQMVFVITVQYGTDTNS | |
| | | | VCPGSGGSGSSQCVNLTTRTQLPPAYTNSFTRGVYYPDKVFRSSVLHSTQDLFLPFFSNVTWFHAIH | |
| | | | VSGTNGTKRFDNPVLPFNDGVYFASTEKSNIIRGWIFGTTLDSKTQSLLIVNNATNVVIKVCEFQFQND | |
| | | | PFLGVYYHKNNKSWMESEFRVYSSANNCTFEYVSQPFLMDLEGKQGNFKNLREFVFKNIDGYFKIYS | |
| | | | KHTPINLVRDLPQGFSALEPLVDLPIGINITRFQTLLALHRSYLTPGDSSSGWTAGAAAYYVGYLQPRTF | |
| | | | LLKYNENGTITDAVDCALDPLSETKCTLKSFTVEKGIYQTSNFRVQPTESIVRFPNITNLCPFGEVFNAT | |
| | | | RFASVYAWNRKRISNCVADYSVLYNSASFSTFKCYGVSPTKLNDLCFTNVYADSFVIRGDEVRQIAPG | |
| | | | QTGKIADYNYKLPDDFTGCVIAWNSNNLDSKVGGNYNYLYRLFRKSNLKPFERDISTEIYQAGSTPCN | |
| | | | GVEGFNCYFPLQSYGFQPTNGVGYQPYRVVVLSFELLHAPATVCGPKKSTNLVKNKCVNFNFNGLTG | |
| | | | TGVLTESNKKFLPFQQFGRDIADTTDAVRDPQTLEILDITPCSFGGVSVITPGTNTSNQVAVLYQDVNC | |
| | | | TEVPVAIHADQLTPTWRVYSTGSNVFQTRAGCLIGAEHVNNSYECDIPIGAGICASYQTQTNSPGSAS | |
| | | | SVASQSIIAYTMSLGAENSVAYSNNSIAIPTNFTISVTTEILPVSMTKTSVDCTMYICGDSTECSNLLLQY | |
| | | | GSFCTQLNRALTGIAVEQDKNTQEVFAQVKQIYKTPPIKDFGGFNFSQILPDPSKPSKRSFIEDLLFNKV | |
| | | | TLADAGFIKQYGDCLGDIAARDLICAQKFNGLTVLPPLLTDEMIAQYTSALLAGTITSGWTFGAGAALQI | |
| | | | PFAMQMAYRFNGIGVTQNVLYENQKLIANQFNSAIGKIQDSLSSTASALGKLQDVVNQNAQALNTLVK | |
| | | | QLSSNFGAISSVLNDILSRLDPPEAEVQIDRLITGRLQSLQTYVTQQLIRAAEIRASANLAATKMSECVLG | |
| | | | QSKRVDFCGKGYHLMSFPQSAPHGVVFLHVTYVPAQEKNFTTAPAICHDGKAHFPREGVFVSNGTH | |
| | | | WFVTQRNFYEPQIITTDNTFVSGNCDVVIGIVNNTVYDPLQSELDSIKEELDKIHKNGSGGSGDIIKLLNE | |
| | | | QVNKEMQSSNLYMSMSSWCYTHSLDGAGLFLFDHAAEEYEHAKKLIIFLNENNVPVQLTSISAPEHKF | |
| | | | EGLTQIFQKAYEHEQHISESINNIVDHAIKSKDHATFNFLQWYVAEQHEEEVLFKDILDKIELIGNENHGL | |
| | | | YLADQYVKGIAKSRKSGS | |
| pCoVP534 | R-SpFN | MER1 (ErinaceusCoV/2012- | ELTECDLDVLFKNDAPIIANYSRRVFTNCNYNLTKLLSLVQVDEFVCHKTTPEALATGCYSSLTVDWFA | 171 |
| | | 174/GER/2012) RBD_SpFN | LPFSMKSTLAIGSAEAISMFNYNQDYSNPTCRIHAAVTANVSTALNFTANANYAYISRCQGVDGKPILLQ | |
| | | | PGQMTNIACRSGVLARPSDADYFGYSFQGRNYYLGRKSYKPKTDEGDVQMVYVITPKYDKGPDTVC | |
| | | | PLKGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATRF | |
| | | | ASVYAWNRKRISNCVADYSVLYNSASFSTFKCYGVSPTKLNDLCFTNVYADSFVIRGDEVRQIAPGQT | |
| | | | GKIADYNYKLPDDFTGCVIAWNSNNLDSKVGGNYNYLYRLFRKSNLKPFERDISTEIYQAGSTPCNGV | |
| | | | EGFNCYFPLQSYGFQPTNGVGYQPYRVVVLSFELLHAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDWVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoVP535 | R-SpFN | MER2 (Neoromicia/5038) | VYPDCNFTELFRERAPTIMQYKREVFTRCNYNLSLLLSLVQVDEFVCDKITPEALATGCYSSLTVDWFA | 172 |
| | | RBD_SpFN | FPYAWKSYLAIGSADRIVRFNYNQDYSNPSCRIHSKVNSSIGISYAGAYSYITNCNYGATNKDDVVKPG | |
| | | | GRASQQCITGALNSPTTGQLWAYNFGGVPYRVSRLTYTDHLSDPLDMVYVITVKYEPGAETVCPKQG | |
| | | | SGGSGSSQCVNLTTRTQLPPAYTNSFTRGVYYPDKVFRSSVLHSTQDLFLPFFSNVTWFHAIHVSGT | |
| | | | NGTKRFDNPVLPFNDGVYFASTEKSNIIRGWIFGTTLDSKTQSLLIVNNATNVVIKVCEFQFCNDPFLGV | |
| | | | YYHKNNKSWMESEFRVYSSANNCTFEYVSQPFLMDLEGKQGNFKNLREFVFKNIDGYFKIYSKHTPIN | |
| | | | LVRDLPQGFSALEPLVDLPIGINITRFQTLLALHRSYLTPGDSSSGWTAGAAAYYVGYLQPRTFLLKYN | |
| | | | ENGTITDAVDCALDPLSETKCTLKSFTVEKGIYQTSNFRVQPTESIVRFPNITNLCPFGEVFNATRFASV | |
| | | | YAWNRKRISNCVADYSVLYNSASFSTFKCYGVSPTKLNDLCFTNVYADSFVIRGDEVRQIAPGQTGKIA | |
| | | | DYNYKLPDDFTGCVIAWNSNNLDSKVGGNYNYLYRLFRKSNLKPFERDISTEIYQAGSTPCNGVEGEN | |
| | | | CYFPLQSYGFQPTNGVGYQPYRVVVLSFELLHAPATVCGPKKSTNLVKNKCVNFNFNGLTGTGVLTE | |
| | | | SNKKFLPFQQFGRDIADTTDAVRDPQTLEILDITPCSFGGVSVITPGTNTSNQVAVLYQDVNCTEVPVAI | |
| | | | HADQLTPTWRVYSTGSNVFQTRAGCLIGAEHVNNSYECDIPIGAGICASYQTQTNSPGSASSVASQSII | |
| | | | AYTMSLGAENSVAYSNNSIAIPTNFTISVTTEILPVSMTKTSVDCTMYICGDSTECSNLLLQYGSFCTQL | |
| | | | NRALTGIAVEQDKNTQEVFAQVKQIYKTPPIKDFGGFNFSQILPDPSKPSKRSFIEDLLFNKVTLADAGFI | |
| | | | KQYGDCLGDIAARDLICAQKFNGLTVLPPLLTDEMIAQYTSALLAGTITSGWTFGAGAALQIPFAMQMA | |
| | | | YRFNGIGVTQNVLYENQKLIANQFNSAIGKIQDSLSSTASALGKLQDVVNQNAQALNTLVKQLSSNFGA | |
| | | | ISSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDVVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP536 | R-SpFN | MER3 (HKU4 SM3A) | QATECDFSPMLTGVAPQVYNFKRLVFSNCNYNLTKLLSLFAVDEFSCNGISPDAIARGCYSTLTVDYFA | 173 |
| | | RBD_SpFN | YPLSMKSYIRPGSAGNIPLYNYKQSFANPTCRVMASVPANVTITKPDAYGYISKCSRLTGANQDVETPL | |
| | | | YINPGEYSICRDFSPGGFSEDGQVFKRTLTQFEGGGLLIGVGTRVPMTDNLQMSFIIFVQYGTGTDSV | |
| | | | CPMLGSGGSGSSQCVNLTTRTQLPPAYTNSFTRGVYYPDKVFRSSVLHSTQDLFLPFFSNVTWFHAI | |
| | | | HVSGTNGTKRFDNPVLPFNDGVYFASTEKSNIIRGWIFGTTLDSKTQSLLIVNNATNVVIKVCEFQFCN | |
| | | | DPFLGVYYHKNNKSWMESEFRVYSSANNCTFEYVSQPFLMDLEGKQGNFKNLREFVFKNIDGYFKIY | |
| | | | SKHTPINLVRDLPQGFSALEPLVDLPIGINITRFQTLLALHRSYLTPGDSSSGWTAGAAAYYVGYLQPRT | |
| | | | FLLKYNENGTITDAVDCALDPLSETKCTLKSFTVEKGIYQTSNFRVQPTESIVRFPNITNLCPFGEVFNA | |
| | | | TRFASVYAWNRKRISNCVADYSVLYNSASFSTFKCYGVSPTKLNDLCFTNVYADSFVIRGDEVRQIAP | |
| | | | GQTGKIADYNYKLPDDFTGCVIAWNSNNLDSKVGGNYNYLYRLFRKSNLKPFERDISTEIYQAGSTPC | |
| | | | NGVEGFNCYFPLQSYGFQPTNGVGYQPYRVVVLSFELLHAPATVCGPKKSTNLVKNKCVNFNFNGLT | |
| | | | GTGVLTESNKKFLPFQQFGRDIADTTDAVRDPQTLEILDITPCSFGGVSVITPGTNTSNQVAVLYQDVN | |
| | | | CTEVPVAIHADQLTPTWRVYSTGSNVFQTRAGCLIGAEHVNNSYECDIPIGAGICASYQTQTNSPGSA | |
| | | | SSVASQSIIAYTMSLGAENSVAYSNNSIAIPTNFTISVTTEILPVSMTKTSVDCTMYICGDSTECSNLLLQ | |
| | | | YGSFCTQLNRALTGIAVEQDKNTQEVFAQVKQIYKTPPIKDFGGFNFSQILPDPSKPSKRSFIEDLLFNK | |
| | | | VTLADAGFIKQYGDCLGDIAARDLICAQKFNGLTVLPPLLTDEMIAQYTSALLAGTITSGWTFGAGAALQ | |
| | | | IPFAMQMAYRFNGIGVTQNVLYENQKLIANQFNSAIGKIQDSLSSTASALGKLQDVVNQNAQALNTLVK | |
| | | | QLSSNFGAISSVLNDILSRLDPPEAEVQIDRLITGRLQSLQTYVTQQLIRAAEIRASANLAATKMSECVLG | |
| | | | QSKRVDFCGKGYHLMSFPQSAPHGVVFLHVTYVPAQEKNFTTAPAICHDGKAHFPREGVFVSNGTH | |
| | | | WFVTQRNFYEPQIITTDNTFVSGNCDWVIGIVNNTVYDPLQSELDSIKEELDKIHKNGSGGSGDIIKLLNE | |
| | | | QVNKEMQSSNLYMSMSSWCYTHSLDGAGLFLFDHAAEEYEHAKKLIIFLNENNVPVQLTSISAPEHKF | |
| | | | EGLTQIFQKAYEHEQHISESINNIVDHAIKSKDHATFNFLQWYVAEQHEEEVLFKDILDKIELIGNENHGL | |
| | | | YLADQYVKGIAKSRKSGS | |
| pCoVP537 | R-SpFN | MER4 (BatCoV-Ita2 206645- | QGVECDFSKLFKAAPPQIYNFSRLVFTNCNYNLTKLLSLFHVSEFSCHQVSPSALASGCYSSLTVDYFA | 174 |
| | | 63) RBD_SpFN | YPLYLASYLQQGSTGEIAQYNYKQDFSNPTCRILASVPANVSIPKPDKYIWLSQCYSFSAYSGDIPHYV | |
| | | | QPGQYTPCLYLTSSGFDKSYQTNRDMQNKMAATGVTSSMTDNLQMAFVISVQYGTDTNSVCPMGS | |
| | | | GGSGSSQCVNLTTRTQLPPAYTNSFTRGVYYPDKVFRSSVLHSTQDLFLPFFSNVTWFHAIHVSGTN | |
| | | | GTKRFDNPVLPFNDGVYFASTEKSNIIRGWIFGTTLDSKTQSLLIVNNATNVVIKVCEFQFCNDPFLGVY | |
| | | | YHKNNKSWMESEFRVYSSANNCTFEYVSQPFLMDLEGKQGNFKNLREFVFKNIDGYFKIYSKHTPINL | |
| | | | VRDLPQGFSALEPLVDLPIGINITRFQTLLALHRSYLTPGDSSSGWTAGAAAYYVGYLQPRTFLLKYNE | |
| | | | NGTITDAVDCALDPLSETKCTLKSFTVEKGIYQTSNFRVQPTESIVRFPNITNLCPFGEVFNATRFASVY | |
| | | | AWNRKRISNCVADYSVLYNSASFSTFKCYGVSPTKLNDLCFTNVYADSFVIRGDEVRQIAPGQTGKIA | |
| | | | DYNYKLPDDFTGCVIAWNSNNLDSKVGGNYNYLYRLFRKSNLKPFERDISTEIYQAGSTPCNGVEGFN | |
| | | | CYFPLQSYGFQPTNGVGYQPYRVVVLSFELLHAPATVCGPKKSTNLVKNKCVNFNFNGLTGTGVLTE | |
| | | | SNKKFLPFQQFGRDIADTTDAVRDPQTLEILDITPCSFGGVSVITPGTNTSNQVAVLYQDVNCTEVPVAI | |
| | | | HADQLTPTWRVYSTGSNVFQTRAGCLIGAEHVNNSYECDIPIGAGICASYQTQTNSPGSASSVASQSII | |
| | | | AYTMSLGAENSVAYSNNSIAIPTNFTISVTTEILPVSMTKTSVDCTMYICGDSTECSNLLLQYGSFCTQL | |
| | | | NRALTGIAVEQDKNTQEVFAQVKQIYKTPPIKDFGGFNFSQILPDPSKPSKRSFIEDLLFNKVTLADAGFI | |
| | | | KQYGDCLGDIAARDLICAQKFNGLTVLPPLLTDEMIAQYTSALLAGTITSGWTFGAGAALQIPFAMQMA | |
| | | | YRFNGIGVTQNVLYENQKLIANQFNSAIGKIQDSLSSTASALGKLQDVVNQNAQALNTLVKQLSSNFGA | |
| | | | ISSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDVVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP538 | R-SpFN | MER5 (BatCoV-Ita1 206645- | QGVECDFSKLFKAAPPQIYNFSRLVFTNCNYNLTKLLSLFHVSEFSCHQVSPSALASGCYSSLTVDYFA | 175 |
| | | 40) RBD_SpFN | YPLYLASYLQQGSTGEIAQYNYKQDFSNPTCRILASVPANVSIPKPDKYIWLSQCYSFSAYSGDVPHYV | |
| | | | LPGQYTPCLYLTSSGFDNSYQTNRDFQNKMAATGVISSMTDNLQMAFVISVQYGTDTNSVCPMGSG | |
| | | | GSGSSQCVNLTTRTQLPPAYTNSFTRGVYYPDKVFRSSVLHSTQDLFLPFFSNVTWFHAIHVSGTNGT | |
| | | | KRFDNPVLPFNDGVYFASTEKSNIIRGWIFGTTLDSKTQSLLIVNNATNWVIKVCEFQFCNDPFLGVYYH | |
| | | | KNNKSWMESEFRVYSSANNCTFEYVSQPFLMDLEGKQGNFKNLREFVFKNIDGYFKIYSKHTPINLVR | |
| | | | DLPQGFSALEPLVDLPIGINITRFQTLLALHRSYLTPGDSSSGWTAGAAAYYVGYLQPRTFLLKYNENG | |
| | | | TITDAVDCALDPLSETKCTLKSFTVEKGIYQTSNFRVQPTESIVRFPNITNLCPFGEVFNATRFASVYAW | |
| | | | NRKRISNCVADYSVLYNSASFSTFKCYGVSPTKLNDLCFTNVYADSFVIRGDEVRQIAPGQTGKIADYN | |
| | | | YKLPDDFTGCVIAWNSNNLDSKVGGNYNYLYRLFRKSNLKPFERDISTEIYQAGSTPCNGVEGENCYF | |
| | | | PLQSYGFQPTNGVGYQPYRVVVLSFELLHAPATVCGPKKSTNLVKNKCVNFNFNGLTGTGVLTESNK | |
| | | | KFLPFQQFGRDIADTTDAVRDPQTLEILDITPCSFGGVSVITPGTNTSNQVAVLYQDVNCTEVPVAIHAD | |
| | | | QLTPTWRVYSTGSNVFQTRAGCLIGAEHVNNSYECDIPIGAGICASYQTQTNSPGSASSVASQSIIAYT | |
| | | | MSLGAENSVAYSNNSIAIPTNFTISVTTEILPVSMTKTSVDCTMYICGDSTECSNLLLQYGSFCTQLNRA | |
| | | | LTGIAVEQDKNTQEVFAQVKQIYKTPPIKDFGGFNFSQILPDPSKPSKRSFIEDLLFNKVTLADAGFIKQY | |
| | | | GDCLGDIAARDLICAQKFNGLTVLPPLLTDEMIAQYTSALLAGTITSGWTFGAGAALQIPFAMQMAYRF | |
| | | | NGIGVTQNVLYENQKLIANQFNSAIGKIQDSLSSTASALGKLQDVVNQNAQALNTLVKQLSSNFGAISS | |
| | | | VLNDILSRLDPPEAEVQIDRLITGRLQSLQTYVTQQLIRAAEIRASANLAATKMSECVLGQSKRVDFCGK | |
| | | | GYHLMSFPQSAPHGVVFLHVTYVPAQEKNFTTAPAICHDGKAHFPREGVFVSNGTHWFVTQRNFYEP | |
| | | | QIITTDNTFVSGNCDVVIGIVNNTVYDPLQSELDSIKEELDKIHKNGSGGSGDIIKLLNEQVNKEMQSSNL | |
| | | | YMSMSSWCYTHSLDGAGLFLFDHAAEEYEHAKKLIIFLNENNVPVQLTSISAPEHKFEGLTQIFQKAYE | |
| | | | HEQHISESINNIVDHAIKSKDHATFNFLQWYVAEQHEEEVLFKDILDKIELIGNENHGLYLADQYVKGIAK | |
| | | | SRKSGS | |
| pCoVP539 | R-SpFN | HKU5 RBD_SpFN | TTQECDFTPMLTGTPPPIYNFKRLVFTNCNYNLTKLLSLFQVSEFSCHQVSPSSLATGCYSSLTVDYFA | 176 |
| | | | YSTDMSSYLQPGSAGAIVQFNYKQDFSNPTCRVLATVPQNLTTITKPSNYAYLTECYKTSAYGKNYLY | |
| | | | NAPGAYTPCLSLASRGFSTKYQSHSDGELTTTGYIYPVTGNLQMAFIISVQYGTDTNSVCPMGSGGSG | |
| | | | SSQCVNLTTRTQLPPAYTNSFTRGVYYPDKVFRSSVLHSTQDLFLPFFSNVTWFHAIHVSGTNGTKRF | |
| | | | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATRFASVYAWNRK | |
| | | | RISNCVADYSVLYNSASFSTFKCYGVSPTKLNDLCFTNVYADSFVIRGDEVRQIAPGQTGKIADYNYKL | |
| | | | PDDFTGCVIAWNSNNLDSKVGGNYNYLYRLFRKSNLKPFERDISTEIYQAGSTPCNGVEGFNCYFPLQ | |
| | | | SYGFQPTNGVGYQPYRVVVLSFELLHAPATVCGPKKSTNLVKNKCVNFNFNGLTGTGVLTESNKKFL | |
| | | | PFQQFGRDIADTTDAVRDPQTLEILDITPCSFGGVSVITPGTNTSNQVAVLYQDVNCTEVPVAIHADQL | |
| | | | TPTWRVYSTGSNVFQTRAGCLIGAEHVNNSYECDIPIGAGICASYQTQTNSPGSASSVASQSIIAYTMS | |
| | | | LGAENSVAYSNNSIAIPTNFTISVTTEILPVSMTKTSVDCTMYICGDSTECSNLLLQYGSFCTQLNRALT | |
| | | | GIAVEQDKNTQEVFAQVKQIYKTPPIKDFGGFNFSQILPDPSKPSKRSFIEDLLFNKVTLADAGFIKQYG | |
| | | | DCLGDIAARDLICAQKFNGLTVLPPLLTDEMIAQYTSALLAGTITSGWTFGAGAALQIPFAMQMAYRFN | |
| | | | GIGVTQNVLYENQKLIANQFNSAIGKIQDSLSSTASALGKLQDVVNQNAQALNTLVKQLSSNFGAISSVL | |
| | | | NDILSRLDPPEAEVQIDRLITGRLQSLQTYVTQQLIRAAEIRASANLAATKMSECVLGQSKRVDFCGKG | |
| | | | YHLMSFPQSAPHGVVFLHVTYVPAQEKNFTTAPAICHDGKAHFPREGVFVSNGTHWFVTQRNFYEPQ | |
| | | | IITTDNTFVSGNCDVVIGIVNNTVYDPLQSELDSIKEELDKIHKNGSGGSGDIIKLLNEQVNKEMQSSNLY | |
| | | | MSMSSWCYTHSLDGAGLFLFDHAAEEYEHAKKLIIFLNENNVPVQLTSISAPEHKFEGLTQIFQKAYEH | |
| | | | EQHISESINNIVDHAIKSKDHATFNFLQWYVAEQHEEEVLFKDILDKIELIGNENHGLYLADQYVKGIAKS | |
| | | | RKSGS | |
| pCoVP540 | R-SpFN | SARS-COV-1 RBD_SpFN | NITNLCPFGEVFNATKFPSVYAWERKKISNCVADYSVLYNSTFFSTFKCYGVSATKLNDLCFSNVYADS | 177 |
| | | | FVVKGDDVRQIAPGQTGVIADYNYKLPDDFMGCVLAWNTRNIDATSTGNYNYKYRYLRHGKLRPFER | |
| | | | DISNVPFSPDGKPCTPPALNCYWPLNDYGFYTTTGIGYQPYRVVVLSFELLNAPATVCGPGSGGGSS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP541 | R-R(Omicron | RBD from Omicron | NITNLCPFDEVFNATRFASVYAWNRKRISNCVADYSVLYNFAPFFAFKCYGVSPTKLNDLCFTNVYADS | 178 |
| | BA4/5)-SpFN | BA.2.12.1_Omi-BA.4/5- | FVIRGNEVSQIAPGQTGNIADYNYKLPDDFTGCVIAWNSNKLDSKVGGNYNYQYRLFRKSNLKPFERD | |
| | | RBD_SpFN | ISTEIYQAGNKPCNGVAGFNCYFPLRSYGFRPTYGVGHQPYRVVVLSFELLHAPATVCGPGSGGSGNI | |
| | | | TNLCPFDEVFNATRFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP542 | R-R(Omicron | RBD from NC_014470. | NITQLCPFNEVFNITSFPSVYAWERMRITNCVADYSVLYNSSASFSTFQCYGVSPTKLNDLCFSSVYAD | 179 |
| | BA4/5)-SpFN | BM4831.BGR.2008_Omi-BA.4/5- | YFVVKGDDVRQIAPAQTGVIADYNYKLPDDFTGCVIAWNTNSLDSSNEFFYRRFRHGKIKPYGRDLSN | |
| | | RBD_SpFN | VLFNPSGGTCSAEGLNCYKPLASYGFTQSSGIGFQPYRVVVLSFELLNAPATVCGPGSGGSGNITNLC | |
| | | | PFDEVFNATRFASVYAWNRKRISNCVADYSVLYNFAPFFAFKCYGVSPTKLNDLCFTNVYADSFVIRG | |
| | | | NEVSQIAPGQTGNIADYNYKLPDDFTGCVIAWNSNKLDSKVGGNYNYRYRLFRKSNLKPFERDISTEIY | |
| | | | QAGNKPCNGVAGVNCYFPLQSYGFRPTYGVGHQPYRVVVLSFELLHAPATVCGPGSGGSGSSQCV | |
| | | | NLTTRTQLPPAYTNSFTRGVYYPDKVFRSSVLHSTQDLFLPFFSNVTWFHAIHVSGTNGTKRFDNPVL | |
| | | | PFNDGVYFASTEKSNIIRGWIFGTTLDSKTQSLLIVNNATNVVIKVCEFQFCNDPFLGVYYHKNNKSWM | |
| | | | ESEFRVYSSANNCTFEYVSQPFLMDLEGKQGNFKNLREFVFKNIDGYFKIYSKHTPINLVRDLPQGFSA | |
| | | | LEPLVDLPIGINITRFQTLLALHRSYLTPGDSSSGWTAGAAAYYVGYLQPRTFLLKYNENGTITDAVDCA | |
| | | | LDPLSETKCTLKSFTVEKGIYQTSNFRVQPTESIVRFPNITNLCPFGEVFNATRFASVYAWNRKRISNC | |
| | | | VADYSVLYNSASFSTFKCYGVSPTKLNDLCFTNVYADSFVIRGDEVRQIAPGQTGKIADYNYKLPDDFT | |
| | | | GCVIAWNSNNLDSKVGGNYNYLYRLFRKSNLKPFERDISTEIYQAGSTPCNGVEGFNCYFPLQSYGFQ | |
| | | | PTNGVGYQPYRVVVLSFELLHAPATVCGPKKSTNLVKNKCVNFNFNGLTGTGVLTESNKKFLPFQQF | |
| | | | GRDIADTTDAVRDPQTLEILDITPCSFGGVSVITPGTNTSNQVAVLYQDVNCTEVPVAIHADQLTPTWR | |
| | | | VYSTGSNVFQTRAGCLIGAEHVNNSYECDIPIGAGICASYQTQTNSPGSASSVASQSIIAYTMSLGAEN | |
| | | | SVAYSNNSIAIPTNFTISVTTEILPVSMTKTSVDCTMYICGDSTECSNLLLQYGSFCTQLNRALTGIAVEQ | |
| | | | DKNTQEVFAQVKQIYKTPPIKDFGGFNFSQILPDPSKPSKRSFIEDLLFNKVTLADAGFIKQYGDCLGDI | |
| | | | AARDLICAQKFNGLTVLPPLLTDEMIAQYTSALLAGTITSGWTFGAGAALQIPFAMQMAYRFNGIGVTQ | |
| | | | NVLYENQKLIANQFNSAIGKIQDSLSSTASALGKLQDVVNQNAQALNTLVKQLSSNFGAISSVLNDILSR | |
| | | | LDPPEAEVQIDRLITGRLQSLQTYVTQQLIRAAEIRASANLAATKMSECVLGQSKRVDFCGKGYHLMSF | |
| | | | PQSAPHGVVFLHVTYVPAQEKNFTTAPAICHDGKAHFPREGVFVSNGTHWFVTQRNFYEPQIITTDNT | |
| | | | FVSGNCDVVIGIVNNTVYDPLQSELDSIKEELDKIHKNGSGGSGDIIKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| pCoVP543 | R-R(Omicron | BM48-31 RBD-T498W_Omi- | NITQLCPFNEVFNITSFPSVYAWERMRITNCVADYSVLYNSSASFSTFQCYGVSPTKLNDLCFSSVYAD | 180 |
| | BA4/5)-SpFN | BA.4/5-RBD_SpFN | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLASYGFWQSSGIGFQPYRVVVLSFELLNAPATVCGPGSGGSGNITNL | |
| | | | CPFDEVFNATRFASVYAWNRKRISNCVADYSVLYNFAPFFAFKCYGVSPTKLNDLCFTNVYADSFVIR | |
| | | | GNEVSQIAPGQTGNIADYNYKLPDDFTGCVIAWNSNKLDSKVGGNYNYRYRLFRKSNLKPFERDISTEI | |
| | | | YQAGNKPCNGVAGVNCYFPLQSYGFRPTYGVGHQPYRVVVLSFELLHAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATRFASVYAWNRKRIS | |
| | | | NCVADYSVLYNSASFSTFKCYGVSPTKLNDLCFTNVYADSFVIRGDEVRQIAPGQTGKIADYNYKLPDD | |
| | | | FTGCVIAWNSNNLDSKVGGNYNYLYRLFRKSNLKPFERDISTEIYQAGSTPCNGVEGFNCYFPLQSYG | |
| | | | FQPTNGVGYQPYRVWVLSFELLHAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP544 | R-R(Omicron | BM48-31 RBD-A493Y- | NITQLCPFNEVFNITSFPSVYAWERMRITNCVADYSVLYNSSASFSTFQCYGVSPTKLNDLCFSSVYAD | 181 |
| | BA4/5)-SpFN | T498W_Omi-BA.4/5-RBD_SpFN | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLYSYGFWQSSGIGFQPYRVVVLSFELLNAPATVCGPGSGGSGNITNL | |
| | | | CPFDEVFNATRFASVYAWNRKRISNCVADYSVLYNFAPFFAFKCYGVSPTKLNDLCFTNVYADSFVIR | |
| | | | GNEVSQIAPGQTGNIADYNYKLPDDFTGCVIAWNSNKLDSKVGGNYNYRYRLFRKSNLKPFERDISTEI | |
| | | | YQAGNKPCNGVAGVNCYFPLQSYGFRPTYGVGHQPYRVVVLSFELLHAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATRFASVYAWNRKRIS | |
| | | | NCVADYSVLYNSASFSTFKCYGVSPTKLNDLCFTNVYADSFVIRGDEVRQIAPGQTGKIADYNYKLPDD | |
| | | | FTGCVIAWNSNNLDSKVGGNYNYLYRLFRKSNLKPFERDISTEIYQAGSTPCNGVEGFNCYFPLQSYG | |
| | | | FQPTNGVGYQPYRVVVLSFELLHAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP545 | R-R(Omicron | RBD from | NITNLCPFGEVFNATTFPSVYAWERKRISNCVADYSVLYNSTSFSTFKCYGVSATKLNDLCFSNVYADS | 182 |
| | BA4/5)-SpFN | KC881005.RsSHC014_Omi- | FVVKGDDVRQIAPGQTGVIADYNYKLPDDFLGCVLAWNTNSKDSSTSGNYNYLYRWVRRSKLNPYER | |
| | | BA.4/5-RBD_SpFN | DLSNDIYSPGGQSCSAVGPNCYNPLRPYGFFTTAGVGHQPYRVVVLSFELLNAPATVCGPGSGGSG | |
| | | | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP546 | R-R(Omicron | RBD from | NITNLCPFGEVFNATTFPSVYAWERKRISNCVADYSVLYNSTSFSTFKCYGVSATKLNDLCFSNVYADS | 183 |
| | BA4/5)-SpFN | KC881007.Bat.WIV1_Omi- | FVVKGDDVRQIAPGQTGVIADYNYKLPDDFTGCVLAWNTRNIDATQTGNYNYKYRSLRHGKLRPFER | |
| | | BA.4/5-RBD_SpFN | DISNVPFSPDGKPCTPPAFNCYWPLNDYGFYITNGIGYQPYRVVVLSFELLNAPATVCGPGSGGSGNI | |
| | | | TNLCPFDEVFNATRFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGFNCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP547 | R-R(Omicron | RBD from AVP78042_ | NITNVCPFHKVFNATRFPSVYAWERTKISDCIADYTVFYNSTSFSTFKCYGVSPSKLIDLCFTSVYADTF | 184 |
| | BA4/5)-SpFN | BatSARSlikecoronavirus_Omi- | LIRFSEVRQVAPGQTGVIADYNYKLPDDFTGCVIAWNTAKQDTGHYFYRSHRSTKLKPFERDLSSDEN | |
| | | BA.4/5-RBD_SpFN | GVRTLSTYDFNPNVPLEYQATRVVVLSFELLNAPATVCGPGSGGSGNITNLCPFDEVFNATRFASVYA | |
| | | | WNRKRISNCVADYSVLYNFAPFFAFKCYGVSPTKLNDLCFTNVYADSFVIRGNEVSQIAPGQTGNIADY | |
| | | | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP548 | R-R(Omicron | RBD from MZ937001_ | NITNLCPFGEVFNATTFASVYAWNRKRISNCVADYSVLYNSTSFSTFKCYGVSPTKLNDLCFTNVYADS | 185 |
| | BA4/5)-SpFN | BANAL20103_Laos_2020_Omi- | FVVRGDEVRQIAPGQTGKIADYNYKLPDDFTGCVIAWNSNNLDSKVGGNYNYLYRLFRKSNLKPFERD | |
| | | BA.4/5-RBD_SpFN | ISTEIYQAGSTPCNGVEGFNCYFPLQSYGFHPTNGVGYQPYRVVVLSFELLNAPATVCGPGSGGSGNI | |
| | | | TNLCPFDEVFNATRFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP549 | R-R(Omicron | RBD from MN996532. | NITNLCPFGEVFNATTFASVYAWNRKRISNCVADYSVLYNSTSFSTFKCYGVSPTKLNDLCFTNVYADS | 186 |
| | BA4/5)-SpFN | 2013.Bat.RaTG13_Omi-BA.4/5- | FVITGDEVRQIAPGQTGKIADYNYKLPDDFTGCVIAWNSKHIDAKEGGNFNYLYRLFRKANLKPFERDI | |
| | | RBD_SpFN | STEIYQAGSKPCNGQTGLNCYYPLYRYGFYPTDGVGHQPYRVVVLSFELLNAPATVCGPGSGGSGNI | |
| | | | TNLCPFDEVFNATRFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP550 | R-R(Omicron | RBD from MZ937004_ | NITNFCPFDKVFNATRFPNVYAWQRTKISDCIADYTVLYNSTSFSTFKCYGVSPSKLIDLCFTSVYADTF | 187 |
| | BA4/5)-SpFN | BANAL20247_Laos_2020_Omi- | LIRFSEVRQIAPGETGVIADYNYKLPDDFTGCVLAWNTAQQDIGSYFYRSHRAVKLKPFERDLSSDEN | |
| | | | GVRTLSTYDFNPNVPLDYQATRVVVLSFELLNAPATVCGPGSGGSGNITNLCPFDEVFNATRFASVYA | |
| | | BA.4/5-RBD_SpFN | WNRKRISNCVADYSVLYNFAPFFAFKCYGVSPTKLNDLCFTNVYADSFVIRGNEVSQIAPGQTGNIADY | |
| | | | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP551 | R-R(Omicron | BANAL-20-247 RBD-N498W_ | NITNFCPFDKVFNATRFPNVYAWQRTKISDCIADYTVLYNSTSFSTFKCYGVSPSKLIDLCFTSVYADTF | 188 |
| | BA4/5)-SpFN | Omi-BA.4/5-RBD_SpFN | LIRFSEVRQIAPGETGVIADYNYKLPDDFTGCVLAWNTAQQDIGSYFYRSHRAVKLKPFERDLSSDEN | |
| | | | GVRTLSTYDFWPNVPLDYQATRVVVLSFELLNAPATVCGPGSGGSGNITNLCPFDEVFNATRFASVYA | |
| | | | WNRKRISNCVADYSVLYNFAPFFAFKCYGVSPTKLNDLCFTNVYADSFVIRGNEVSQIAPGQTGNIADY | |
| | | | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNWVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP552 | R-R(Omicron | BANAL-20-247 RBD-S493Y- | NITNFCPFDKVFNATRFPNVYAWQRTKISDCIADYTVLYNSTSFSTFKCYGVSPSKLIDLCFTSVYADTF | 189 |
| | BA4/5)-SpFN | N498W_Omi-BA.4/5-RBD_SpFN | LIRFSEVRQIAPGETGVIADYNYKLPDDFTGCVLAWNTAQQDIGSYFYRSHRAVKLKPFERDLSSDEN | |
| | | | GVRTLYTYDFWPNVPLDYQATRVVVLSFELLNAPATVCGPGSGGSGNITNLCPFDEVFNATRFASVYA | |
| | | | WNRKRISNCVADYSVLYNFAPFFAFKCYGVSPTKLNDLCFTNVYADSFVIRGNEVSQIAPGQTGNIADY | |
| | | | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP553 | R-R(Omicron | RBD from ATO98145_ | NITNLCPFDAVFNATRFPSVYAWERVKISNCVADYTAFYNSTSFSTFKCYGVSPSKLIDLCFTSVYADT | 190 |
| | BA4/5)-SpFN | BatSARSlikecoronavirus_Omi- | FLIRFSEVRQVAPGETGVIADYNYKLPDDFTGCVIAWNTAKQDVGSYFYRSHRSSKLKPFERDLSSDE | |
| | | BA.4/5-RBD_SpFN | NGVRTLSTYDFNPNVPLDYQATRVVVLSFELLNAPATVCGPGSGGSGNITNLCPFDEVFNATRFASVY | |
| | | | AWNRKRISNCVADYSVLYNFAPFFAFKCYGVSPTKLNDLCFTNVYADSFVIRGNEVSQIAPGQTGNIAD | |
| | | | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP554 | R-R(Omicron | RBD from JX993987. | NITNRCPFDKVFNATRFPSVYAWERTKISDCVADYTVLYNSTSFSTFKCYGVSPSKLIDLCFTSVYADT | 191 |
| | BA4/5)-SpFN | 2011.BatRp.Shaanxi2011_Omi- | FLIRSSEVRQVAPGETGVIADYNYKLPDDFTGCVIAWNTANQDQGQYYYRSSRKEKLKPFERDLSSDE | |
| | | BA.4/5-RBD_SpFN | NGVYTLSTYDFYPSVPLDYQATRVVVLSFELLNAPATVCGPGSGGSGNITNLCPFDEVFNATRFASVY | |
| | | | AWNRKRISNCVADYSVLYNFAPFFAFKCYGVSPTKLNDLCFTNVYADSFVIRGNEVSQIAPGQTGNIAD | |
| | | | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP555 | R-R(Omicron | RBD from KJ473812. | NITNLCPFDKVFNATRFPSVYAWERTKISDCVADYTVFYNSTSFSTFNCYGVSPSKLIDLCFTSVYADT | 192 |
| | BA4/5)-SpFN | BtRfBetaCoV.HeB2013_Omi- | FLIRFSEVRQVAPGQTGVIADYNYKLPDDFTGCVIAWNTAKQDVGSYFYRSHRSSKLKPFERDLSSEE | |
| | | BA.4/5-RBD_SpFN | NGVRTLSTYDFNQYVPLEYQATRVVVLSFELLNAPATVCGPGSGGSGNITNLCPFDEVFNATRFASVY | |
| | | | AWNRKRISNCVADYSVLYNFAPFFAFKCYGVSPTKLNDLCFTNVYADSFVIRGNEVSQIAPGQTGNIAD | |
| | | | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRENGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP556 | R-R(Omicron | RBD from DQ071615.2003. | NITNRCPFDKVFNATRFPNVYAWERTKISDCVADYTVLYNSTSFSTFKCYGVSPSKLIDLCFTSVYADT | 193 |
| | BA4/5)-SpFN | Bat.Rp3_Omi-BA.4/5-RBD_ | FLIRSSEVRQVAPGETGVIADYNYKLPDDFTGCVIAWNTAKQDQGQYYYRSHRKTKLKPFERDLSSDE | |
| | | SpFN | NGVRTLSTYDFYPSVPVAYQATRVVVLSFELLNAPATVCGPGSGGSGNITNLCPFDEVFNATRFASVY | |
| | | | AWNRKRISNCVADYSVLYNFAPFFAFKCYGVSPTKLNDLCFTNVYADSFVIRGNEVSQIAPGQTGNIAD | |
| | | | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP557 | R-R(Omicron | RBD from | NITNRCPFDKVFNASRFPNVYAWERTKISDCVADYTVLYNSTSFSTFKCYGVSPSKLIDLCFTSVYADT | 194 |
| | BA4/5)-SpFN | ACU31032_Rs_672_2006_Omi- | FLIRSSEVRQVAPGETGVIADYNYKLPDDFTGCVIAWNTAKQDQGQYYYRSSRKTKLKPFERDLTSDE | |
| | | BA.4/5-RBD_SpFN | NGVRTLSTYDFYPNVPIEYQATRVVVLSFELLNAPATVCGPGSGGSGNITNLCPFDEVFNATRFASVY | |
| | | | AWNRKRISNCVADYSVLYNFAPFFAFKCYGVSPTKLNDLCFTNVYADSFVIRGNEVSQIAPGQTGNIAD | |
| | | | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATRFASVYAWNRKRISNCVADYSVLYNSASFSTFK | |
| | | | CYGVSPTKLNDLCFTNVYADSFVIRGDEVRQIAPGQTGKIADYNYKLPDDFTGCVIAWNSNNLDSKVG | |
| | | | GNYNYLYRLFRKSNLKPFERDISTEIYQAGSTPCNGVEGFNCYFPLQSYGFQPTNGVGYQPYRVVVLS | |
| | | | FELLHAPATVCGPKKSTNLVKNKCVNFNFNGLTGTGVLTESNKKFLPFQQFGRDIADTTDAVRDPQTL | |
| | | | EILDITPCSFGGVSVITPGTNTSNQVAVLYQDVNCTEVPVAIHADQLTPTWRVYSTGSNVFQTRAGCLI | |
| | | | GAEHVNNSYECDIPIGAGICASYQTQTNSPGSASSVASQSIIAYTMSLGAENSVAYSNNSIAIPTNFTISV | |
| | | | TTEILPVSMTKTSVDCTMYICGDSTECSNLLLQYGSFCTQLNRALTGIAVEQDKNTQEVFAQVKQIYKT | |
| | | | PPIKDFGGFNFSQILPDPSKPSKRSFIEDLLFNKVTLADAGFIKQYGDCLGDIAARDLICAQKFNGLTVLP | |
| | | | PLLTDEMIAQYTSALLAGTITSGWTFGAGAALQIPFAMQMAYRFNGIGVTQNVLYENQKLIANQFNSAI | |
| | | | GKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP558 | R-R(Omicron | RBD from MZ081380_ | NITNLCPFSQVFNATRFPSVYAWTRERISNCIADYSVLYNSTSFSTFRCYGVSPTKLNDLCFSNVYADS | 195 |
| | BA4/5)-SpFN | bat_Yunnan_RsYN04_ | MWVRGDEVRQIAPSQTGVIADYNYKLPDDFTGCVIAWNSKAKDENGQYFYRLFRKSKLLPFQRDVSN | |
| | | 2020_Omi-BA.4/5-RBD_SpFN | VTYGSGKNDGCNPSEADCYWPLLKYGFTSSVSQDYQPYRVVVLSFELLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGENCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP559 | R-R(Omicron | RsYN04 RBD-T498W_Omi- | NITNLCPFSQVFNATRFPSVYAWTRERISNCIADYSVLYNSTSFSTFRCYGVSPTKLNDLCFSNVYADS | 196 |
| | BA4/5)-SpFN | BA.4/5-RBD_SpFN | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFWSSVSQDYQPYRVVVLSFELLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVWVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP560 | R-R(Omicron | RsYN04 RBD-L493Y-T498W_ | NITNLCPFSQVFNATRFPSVYAWTRERISNCIADYSVLYNSTSFSTFRCYGVSPTKLNDLCFSNVYADS | 197 |
| | SBA4/5)-pFN | Omi-BA.4/5-RBD_SpFN | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLYKYGFWSSVSQDYQPYRVVVLSFELLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGENCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP561 | R-R(Omicron | Khosta2-MZ190138_Omi- | NMTNICPFDQVFNKTQFPSVYAWERVRISDCVSDYTVLYNSSASFSTFKCYGVSPTKLNDLCFSGVYA | 198 |
| | BA4/5)-SpFN | BA.4/5-RBD_SpFN | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFTSTSGVGYQPFRVVVLSFELLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP562 | R-R(Omicron | Khosta-2 RBD-T498W_Omi- | NMTNICPFDQVFNKTQFPSVYAWERVRISDCVSDYTVLYNSSASFSTFKCYGVSPTKLNDLCFSGVYA | 199 |
| | SBA4/5)-pFN | BA.4/5-RBD_SpFN | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFWSTSGVGYQPFRVVVLSFELLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGENCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP563 | R-R(Omicron | Khosta-2 RBD-Q493Y- | NMTNICPFDQVFNKTQFPSVYAWERVRISDCVSDYTVLYNSSASFSTFKCYGVSPTKLNDLCFSGVYA | 200 |
| | BA4/5)-SpFN | T498W_Omi-BA.4/5-RBD_SpFN | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLYDYGFWSTSGVGYQPFRVVVLSFELLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP564 | R-R(Omicron | KY352407_BtKY72_bat_K | NITNLCPFGQVFNASNFPSVYAWERLRISDCVADYAVLYNSSSSFSTFKCYGVSPTKLNDLCFSSVYA | 201 |
| | BA4/5)-SpFN | enya_Omi-BA.4/5-RBD_SpFN | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFTPTVGVGYQPYRVVVLSFELLNAPATVCGPGSGGSGN | |
| | | | ITNLCPFDEVFNATRFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP565 | R-R(Omicron | BtKY72 RBD-T498W_Omi- | NITNLCPFGQVFNASNFPSVYAWERLRISDCVADYAVLYNSSSSFSTFKCYGVSPTKLNDLCFSSVYA | 202 |
| | BA4/5)-SpFN | BA.4/5-RBD_SpFN | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFWPTVGVGYQPYRVVVLSFELLNAPATVCGPGSGGSG | |
| | | | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP566 | R-R(Omicron | BtKY72 RBD-K493Y-T498W_ | NITNLCPFGQVFNASNFPSVYAWERLRISDCVADYAVLYNSSSSFSTFKCYGVSPTKLNDLCFSSVYA | 203 |
| | BA4/5)-SpFN | Omi-BA.4/5-RBD_SpFN | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLYSYGFWPTVGVGYQPYRVVVLSFELLNAPATVCGPGSGGSG | |
| | | | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP567 | R-R(Omicron | RaTG15_Ra7909 (RBD)_Omi- | NITNLCPFSQVFNATRFPSVYAWTRERISNCIADYSVLYNSTSFSTFRCYGVSPTKLNDLCFSNVYADS | 204 |
| | BA4/5)-SpFN | BA.4/5-RBD_SpFN | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFTGSVSQDYQPYRVVVLSFELLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP568 | R-R(Omicron | HKU3-1_DQ022305.2005 | NITNRCPFDKVFNATRFPNVYAWERTKISDCVADYTVLYNSTSFSTFKCYGVSPSKLIDLCFTSVYADT | 205 |
| | BA4/5)-SpFN | (RBD)_Omi-BA.4/5-RBD_SpFN | FLIRSSEVRQVAPGETGVIADYNYKLPDDFTGCVIAWNTAKHDTGNYYYRSHRKTKLKPFERDLSSDD | |
| | | | GNGVYTLSTYDFNPNVPVAYQATRVVVLSFELLNAPATVCGPGSGGSGNITNLCPFDEVFNATRFASV | |
| | | | YAWNRKRISNCVADYSVLYNFAPFFAFKCYGVSPTKLNDLCFTNVYADSFVIRGNEVSQIAPGQTGNIA | |
| | | | DYNYKLPDDFTGCVIAWNSNKLDSKVGGNYNYRYRLFRKSNLKPFERDISTEIYQAGNKPCNGVAGV | |
| | | | NCYFPLQSYGFRPTYGVGHQPYRVVVLSFELLHAPATVCGPGSGGSGSSQCVNLTTRTQLPPAYTNS | |
| | | | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATRFASVYAWNRKRISNCVADYSVLYNSASFSTF | |
| | | | KCYGVSPTKLNDLCFTNVYADSFVIRGDEVRQIAPGQTGKIADYNYKLPDDFTGCVIAWNSNNLDSKV | |
| | | | GGNYNYLYRLFRKSNLKPFERDISTEIYQAGSTPCNGVEGFNCYFPLQSYGFQPTNGVGYQPYRVVV | |
| | | | LSFELLHAPATVCGPKKSTNLVKNKCVNFNFNGLTGTGVLTESNKKFLPFQQFGRDIADTTDAVRDPQ | |
| | | | TLEILDITPCSFGGVSVITPGTNTSNQVAVLYQDVNCTEVPVAIHADQLTPTWRVYSTGSNVFQTRAGC | |
| | | | LIGAEHVNNSYECDIPIGAGICASYQTQTNSPGSASSVASQSIIAYTMSLGAENSVAYSNNSIAIPTNFTI | |
| | | | SVTTEILPVSMTKTSVDCTMYICGDSTECSNLLLQYGSFCTQLNRALTGIAVEQDKNTQEVFAQVKQIY | |
| | | | KTPPIKDFGGFNFSQILPDPSKPSKRSFIEDLLFNKVTLADAGFIKQYGDCLGDIAARDLICAQKFNGLTV | |
| | | | LPPLLTDEMIAQYTSALLAGTITSGWTFGAGAALQIPFAMQMAYRFNGIGVTQNVLYENQKLIANQFNS | |
| | | | AIGKIQDSLSSTASALGKLQDVVNQNAQALNTLVKQLSSNFGAISSVLNDILSRLDPPEAEVQIDRLITGR | |
| | | | LQSLQTYVTQQLIRAAEIRASANLAATKMSECVLGQSKRVDFCGKGYHLMSFPQSAPHGVVFLHVTYV | |
| | | | PAQEKNFTTAPAICHDGKAHFPREGVFVSNGTHWFVTQRNFYEPQIITTDNTFVSGNCDWVIGIVNNTV | |
| | | | YDPLQSELDSIKEELDKIHKNGSGGSGDIIKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP569 | R-R(Omicron | RhGB01-MW719567 (RBD)_ | NVTQLCPFSDVFNRTMFPSVYAWERMSIRDCVADYAVLYNSSISFSTFECYGVSPTKLNDLCFSSVYA | 206 |
| | BA4/5)-SpFN | Omi-BA.4/5-RBD_SpFN | DYFWVRGDDVKQIAPGKTGVIADYNYKLPDDFVGCVLAWNSRHVDNKQGFYYRLFRHGKIKPYQRDM | |
| | | | SRELYSPSGTPCTVVGFNCYDPLAHYAFASPGIGIGYQPFRVVVLSFQLLNAPATVCGPGSGGSGNIT | |
| | | | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP570 | R-R(Omicron | bioinformatically derived | NITNLCPFDEVFNATRFPSVYAWERKKISNCVADYSVLYNSTSFSTFKCYGVSPSKLNDLCFTSVYADS | 207 |
| | BA4/5)-SpFN | Cluster1_RBD (SARS- | FVIRGDEVRQIAPGQTGVIADYNYKLPDDFTGCVIAWNTANQDSGEGGNPNYYYRSSRKSKLKPFER | |
| | | related)_Omi-BA.4/5- | DLSSDEYNVGSQPCNRMGPNCYRTLSTYDFYPTVGVGYQPYRVVVLSFELLNAPATVCGPGSGGSG | |
| | | RBD_SpFN | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP571 | R-R(Omicron | bioinformatically derived | NLPDCNIEEWLTAKSVPSPLNWERKTFSNCNFNLSSLLRFVQADSFSCNNIDASKVYGMCFGSISVDK | 208 |
| | BA4/5)-SpFN | Cluster2_RBD (OC43- | FAIPNSRQVDLQLGNSGFLQSFNYKIDTTATSCQLYYSLPANNVTINNYNPSSWNRRYGFNDDTVFKP | |
| | | related)_Omni-BA.4/5- | RPAGVLTNHDVVYSQQCFKVPNTFCPCKNNGSCVSSGKGTTNGSGTCPAGTNYRTCENTTWCACS | |
| | | RBD_SpFN | CTPDPITASGPRKCPQKKSLVGVGEHCSGLGVDSDYCGNVSCLCSTDAFLGWSADSCLVNDRCNIFA | |
| | | | NIILNGINSGTTCSTGSGGSGNITNLCPFDEVFNATRFASVYAWNRKRISNCVADYSVLYNFAPFFAFK | |
| | | | CYGVSPTKLNDLCFTNVYADSFVIRGNEVSQIAPGQTGNIADYNYKLPDDFTGCVIAWNSNKLDSKVG | |
| | | | GNYNYRYRLFRKSNLKPFERDISTEIYQAGNKPCNGVAGVNCYFPLQSYGFRPTYGVGHQPYRVVVL | |
| | | | SFELLHAPATVCGPGSGGSGSSQCVNLTTRTQLPPAYTNSFTRGVYYPDKVFRSSVLHSTQDLFLPFF | |
| | | | SNVTWFHAIHVSGTNGTKRFDNPVLPFNDGVYFASTEKSNIIRGWIFGTTLDSKTQSLLIVNNATNVVIK | |
| | | | VCEFQFCNDPFLGVYYHKNNKSWMESEFRVYSSANNCTFEYVSQPFLMDLEGKQGNFKNLREFVFK | |
| | | | NIDGYFKIYSKHTPINLVRDLPQGFSALEPLVDLPIGINITRFQTLLALHRSYLTPGDSSSGWTAGAAAYY | |
| | | | VGYLQPRTFLLKYNENGTITDAVDCALDPLSETKCTLKSFTVEKGIYQTSNFRVQPTESIVRFPNITNLC | |
| | | | PFGEVFNATRFASVYAWNRKRISNCVADYSVLYNSASFSTFKCYGVSPTKLNDLCFTNVYADSFVIRG | |
| | | | DEVRQIAPGQTGKIADYNYKLPDDFTGCVIAWNSNNLDSKVGGNYNYLYRLFRKSNLKPFERDISTEIY | |
| | | | QAGSTPCNGVEGFNCYFPLQSYGFQPTNGVGYQPYRVVVLSFELLHAPATVCGPKKSTNLVKNKCV | |
| | | | NFNFNGLTGTGVLTESNKKFLPFQQFGRDIADTTDAVRDPQTLEILDITPCSFGGVSVITPGTNTSNQV | |
| | | | AVLYQDVNCTEVPVAIHADQLTPTWRVYSTGSNVFQTRAGCLIGAEHVNNSYECDIPIGAGICASYQT | |
| | | | QTNSPGSASSVASQSIIAYTMSLGAENSVAYSNNSIAIPTNFTISVTTEILPVSMTKTSVDCTMYICGDST | |
| | | | ECSNLLLQYGSFCTQLNRALTGIAVEQDKNTQEVFAQVKQIYKTPPIKDFGGFNFSQILPDPSKPSKRS | |
| | | | FIEDLLFNKVTLADAGFIKQYGDCLGDIAARDLICAQKFNGLTVLPPLLTDEMIAQYTSALLAGTITSGWT | |
| | | | FGAGAALQIPFAMQMAYRFNGIGVTQNVLYENQKLIANQFNSAIGKIQDSLSSTASALGKLQDVVNQN | |
| | | | AQALNTLVKQLSSNFGAISSVLNDILSRLDPPEAEVQIDRLITGRLQSLQTYVTQQLIRAAEIRASANLAA | |
| | | | TKMSECVLGQSKRVDFCGKGYHLMSFPQSAPHGVVFLHVTYVPAQEKNFTTAPAICHDGKAHFPREG | |
| | | | VFVSNGTHWFVTQRNFYEPQIITTDNTFVSGNCDVVIGIVNNTVYDPLQSELDSIKEELDKIHKNGSGG | |
| | | | SGDIIKLLNEQVNKEMQSSNLYMSMSSWCYTHSLDGAGLFLFDHAAEEYEHAKKLIIFLNENNVPVQLT | |
| | | | SISAPEHKFEGLTQIFQKAYEHEQHISESINNIVDHAIKSKDHATFNFLQWYVAEQHEEEVLFKDILDKIE | |
| | | | LIGNENHGLYLADQYVKGIAKSRKSGS | |
| pCoVP572 | R-R(Omicron | bioinformatically derived | QGVECDFSKLFKAAAPQIYNFSRLVFTNCNYNLTKLLSLFQVDEFSCHQISPSALASGCYSSLTVDYFA | 209 |
| | BA4/5)-SpFN | Cluster3_RBD (MERS- | YPLSMKSYLQIGSAGEISQFNYKQDFSNPTCRILASVPANVSITKPDKYIYISQCYSLSAYSDVDVPHYV | |
| | | related)_Omi-BA.4/5- | NPGQYTSIPCLYLTSSGFDEDGQYFRYNLTQFEGQNYLVATGVTVSMTDNLQMVFVITVQYGTDTNS | |
| | | RBD_SpFN | VCPGSGGSGNITNLCPFDEVFNATRFASVYAWNRKRISNCVADYSVLYNFAPFFAFKCYGVSPTKLND | |
| | | | LCFTNVYADSFVIRGNEVSQIAPGQTGNIADYNYKLPDDFTGCVIAWNSNKLDSKVGGNYNYRYRLFR | |
| | | | KSNLKPFERDISTEIYQAGNKPCNGVAGVNCYFPLQSYGFRPTYGVGHQPYRVVVLSFELLHAPATVC | |
| | | | GPGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATRF | |
| | | | ASVYAWNRKRISNCVADYSVLYNSASFSTFKCYGVSPTKLNDLCFTNVYADSFVIRGDEVRQIAPGQT | |
| | | | GKIADYNYKLPDDFTGCVIAWNSNNLDSKVGGNYNYLYRLFRKSNLKPFERDISTEIYQAGSTPCNGV | |
| | | | EGFNCYFPLQSYGFQPTNGVGYQPYRVVVLSFELLHAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDVVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoVP573 | R-R(Omicron | MER1 (ErinaceusCoV/2012- | ELTECDLDVLFKNDAPIIANYSRRVFTNCNYNLTKLLSLVQVDEFVCHKTTPEALATGCYSSLTVDWFA | 210 |
| | BA4/5)-SpFN | 174/GER/2012) RBD_Omi- | LPFSMKSTLAIGSAEAISMFNYNQDYSNPTCRIHAAVTANVSTALNFTANANYAYISRCQGVDGKPILLQ | |
| | | BA.4/5-RBD_SpFN | PGQMTNIACRSGVLARPSDADYFGYSFQGRNYYLGRKSYKPKTDEGDVQMVYVITPKYDKGPDTVC | |
| | | | PLKGSGGSGNITNLCPFDEVFNATRFASVYAWNRKRISNCVADYSVLYNFAPFFAFKCYGVSPTKLND | |
| | | | LCFTNVYADSFVIRGNEVSQIAPGQTGNIADYNYKLPDDFTGCVIAWNSNKLDSKVGGNYNYRYRLFR | |
| | | | KSNLKPFERDISTEIYQAGNKPCNGVAGVNCYFPLQSYGFRPTYGVGHQPYRVWVLSFELLHAPATVC | |
| | | | GPGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATRF | |
| | | | ASVYAWNRKRISNCVADYSVLYNSASFSTFKCYGVSPTKLNDLCFTNVYADSFVIRGDEVRQIAPGQT | |
| | | | GKIADYNYKLPDDFTGCVIAWNSNNLDSKVGGNYNYLYRLFRKSNLKPFERDISTEIYQAGSTPCNGV | |
| | | | EGFNCYFPLQSYGFQPTNGVGYQPYRVVVLSFELLHAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDWVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoVP574 | R-R(Omicron | MER2 (Neoromicia/5038) | VYPDCNFTELFRERAPTIMQYKREVFTRCNYNLSLLLSLVQVDEFVCDKITPEALATGCYSSLTVDWFA | 211 |
| | BA4/5)-SpFN | RBD_Omi-BA.4/5-RBD_SpFN | FPYAWKSYLAIGSADRIVRFNYNQDYSNPSCRIHSKVNSSIGISYAGAYSYITNCNYGATNKDDVVKPG | |
| | | | GRASQQCITGALNSPTTGQLWAYNFGGVPYRVSRLTYTDHLSDPLDMVYVITVKYEPGAETVCPKQG | |
| | | | SGGSGNITNLCPFDEVFNATRFASVYAWNRKRISNCVADYSVLYNFAPFFAFKCYGVSPTKLNDLCFT | |
| | | | NVYADSFVIRGNEVSQIAPGQTGNIADYNYKLPDDFTGCVIAWNSNKLDSKVGGNYNYRYRLFRKSNL | |
| | | | KPFERDISTEIYQAGNKPCNGVAGVNCYFPLQSYGFRPTYGVGHQPYRVVVLSFELLHAPATVCGPG | |
| | | | SGGSGSSQCVNLTTRTQLPPAYTNSFTRGVYYPDKVFRSSVLHSTQDLFLPFFSNVTWFHAIHVSGT | |
| | | | NGTKRFDNPVLPFNDGVYFASTEKSNIIRGWIFGTTLDSKTQSLLIVNNATNVVIKVCEFQFCNDPFLGV | |
| | | | YYHKNNKSWMESEFRVYSSANNCTFEYVSQPFLMDLEGKQGNFKNLREFVFKNIDGYFKIYSKHTPIN | |
| | | | LVRDLPQGFSALEPLVDLPIGINITRFQTLLALHRSYLTPGDSSSGWTAGAAAYYVGYLQPRTFLLKYN | |
| | | | ENGTITDAVDCALDPLSETKCTLKSFTVEKGIYQTSNFRVQPTESIVRFPNITNLCPFGEVFNATRFASV | |
| | | | YAWNRKRISNCVADYSVLYNSASFSTFKCYGVSPTKLNDLCFTNVYADSFVIRGDEVRQIAPGQTGKIA | |
| | | | DYNYKLPDDFTGCVIAWNSNNLDSKVGGNYNYLYRLFRKSNLKPFERDISTEIYQAGSTPCNGVEGEN | |
| | | | CYFPLQSYGFQPTNGVGYQPYRVVVLSFELLHAPATVCGPKKSTNLVKNKCVNFNFNGLTGTGVLTE | |
| | | | SNKKFLPFQQFGRDIADTTDAVRDPQTLEILDITPCSFGGVSVITPGTNTSNQVAVLYQDVNCTEVPVAI | |
| | | | HADQLTPTWRVYSTGSNVFQTRAGCLIGAEHVNNSYECDIPIGAGICASYQTQTNSPGSASSVASQSII | |
| | | | AYTMSLGAENSVAYSNNSIAIPTNFTISVTTEILPVSMTKTSVDCTMYICGDSTECSNLLLQYGSFCTQL | |
| | | | NRALTGIAVEQDKNTQEVFAQVKQIYKTPPIKDFGGFNFSQILPDPSKPSKRSFIEDLLFNKVTLADAGFI | |
| | | | KQYGDCLGDIAARDLICAQKFNGLTVLPPLLTDEMIAQYTSALLAGTITSGWTFGAGAALQIPFAMQMA | |
| | | | YRFNGIGVTQNVLYENQKLIANQFNSAIGKIQDSLSSTASALGKLQDVVNQNAQALNTLVKQLSSNFGA | |
| | | | ISSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDVVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP575 | R-R(Omicron | MER3 (HKU4 SM3A) RBD_ | QATECDFSPMLTGVAPQVYNFKRLVFSNCNYNLTKLLSLFAVDEFSCNGISPDAIARGCYSTLTVDYFA | 212 |
| | BA4/5)-SpFN | Omi-BA.4/5-RBD_SpFN | YPLSMKSYIRPGSAGNIPLYNYKQSFANPTCRVMASVPANVTITKPDAYGYISKCSRLTGANQDVETPL | |
| | | | YINPGEYSICRDFSPGGFSEDGQVFKRTLTQFEGGGLLIGVGTRVPMTDNLQMSFIIFVQYGTGTDSV | |
| | | | CPMLGSGGSGNITNLCPFDEVFNATRFASVYAWNRKRISNCVADYSVLYNFAPFFAFKCYGVSPTKLN | |
| | | | DLCFTNVYADSFVIRGNEVSQIAPGQTGNIADYNYKLPDDFTGCVIAWNSNKLDSKVGGNYNYRYRLF | |
| | | | RKSNLKPFERDISTEIYQAGNKPCNGVAGVNCYFPLQSYGFRPTYGVGHQPYRVVVLSFELLHAPATV | |
| | | | CGPGSGGSGSSQCVNLTTRTQLPPAYTNSFTRGVYYPDKVFRSSVLHSTQDLFLPFFSNVTWFHAIH | |
| | | | VSGTNGTKRFDNPVLPFNDGVYFASTEKSNIIRGWIFGTTLDSKTQSLLIVNNATNVVIKVCEFQFQND | |
| | | | PFLGVYYHKNNKSWMESEFRVYSSANNCTFEYVSQPFLMDLEGKQGNFKNLREFVFKNIDGYFKIYS | |
| | | | KHTPINLVRDLPQGFSALEPLVDLPIGINITRFQTLLALHRSYLTPGDSSSGWTAGAAAYYVGYLQPRTF | |
| | | | LLKYNENGTITDAVDCALDPLSETKCTLKSFTVEKGIYQTSNFRVQPTESIVRFPNITNLCPFGEVFNAT | |
| | | | RFASVYAWNRKRISNCVADYSVLYNSASFSTFKCYGVSPTKLNDLCFTNVYADSFVIRGDEVRQIAPG | |
| | | | QTGKIADYNYKLPDDFTGCVIAWNSNNLDSKVGGNYNYLYRLFRKSNLKPFERDISTEIYQAGSTPCN | |
| | | | GVEGFNCYFPLQSYGFQPTNGVGYQPYRVVVLSFELLHAPATVCGPKKSTNLVKNKCVNFNFNGLTG | |
| | | | TGVLTESNKKFLPFQQFGRDIADTTDAVRDPQTLEILDITPCSFGGVSVITPGTNTSNQVAVLYQDVNC | |
| | | | TEVPVAIHADQLTPTWRVYSTGSNVFQTRAGCLIGAEHVNNSYECDIPIGAGICASYQTQTNSPGSAS | |
| | | | SVASQSIIAYTMSLGAENSVAYSNNSIAIPTNFTISVTTEILPVSMTKTSVDCTMYICGDSTECSNLLLQY | |
| | | | GSFCTQLNRALTGIAVEQDKNTQEVFAQVKQIYKTPPIKDFGGFNFSQILPDPSKPSKRSFIEDLLFNKV | |
| | | | TLADAGFIKQYGDCLGDIAARDLICAQKFNGLTVLPPLLTDEMIAQYTSALLAGTITSGWTFGAGAALQI | |
| | | | PFAMQMAYRFNGIGVTQNVLYENQKLIANQFNSAIGKIQDSLSSTASALGKLQDVVNQNAQALNTLVK | |
| | | | QLSSNFGAISSVLNDILSRLDPPEAEVQIDRLITGRLQSLQTYVTQQLIRAAEIRASANLAATKMSECVLG | |
| | | | QSKRVDFCGKGYHLMSFPQSAPHGVVFLHVTYVPAQEKNFTTAPAICHDGKAHFPREGVFVSNGTH | |
| | | | WFVTQRNFYEPQIITTDNTFVSGNCDWVIGIVNNTVYDPLQSELDSIKEELDKIHKNGSGGSGDIIKLLNE | |
| | | | QVNKEMQSSNLYMSMSSWCYTHSLDGAGLFLFDHAAEEYEHAKKLIIFLNENNVPVQLTSISAPEHKF | |
| | | | EGLTQIFQKAYEHEQHISESINNIVDHAIKSKDHATFNFLQWYVAEQHEEEVLFKDILDKIELIGNENHGL | |
| | | | YLADQYVKGIAKSRKSGS | |
| pCoVP576 | R-R(Omicron | MER4 (BatCoV-Ita2 206645- | QGVECDFSKLFKAAPPQIYNFSRLVFTNCNYNLTKLLSLFHVSEFSCHQVSPSALASGCYSSLTVDYFA | 213 |
| | BA4/5)-SpFN | 63) RBD_Omi-BA.4/5- | YPLYLASYLQQGSTGEIAQYNYKQDFSNPTCRILASVPANVSIPKPDKYIWLSQCYSFSAYSGDIPHYV | |
| | | RBD_SpFN | QPGQYTPCLYLTSSGFDKSYQTNRDMQNKMAATGVTSSMTDNLQMAFVISVQYGTDTNSVCPMGS | |
| | | | GGSGNITNLCPFDEVFNATRFASVYAWNRKRISNCVADYSVLYNFAPFFAFKCYGVSPTKLNDLCFTN | |
| | | | VYADSFVIRGNEVSQIAPGQTGNIADYNYKLPDDFTGCVIAWNSNKLDSKVGGNYNYRYRLFRKSNLK | |
| | | | PFERDISTEIYQAGNKPCNGVAGVNCYFPLQSYGFRPTYGVGHQPYRVVVLSFELLHAPATVCGPGS | |
| | | | GGSGSSQCVNLTTRTQLPPAYTNSFTRGVYYPDKVFRSSVLHSTQDLFLPFFSNVTWFHAIHVSGTN | |
| | | | GTKRFDNPVLPFNDGVYFASTEKSNIIRGWIFGTTLDSKTQSLLIVNNATNVVIKVCEFQFCNDPFLGVY | |
| | | | YHKNNKSWMESEFRVYSSANNCTFEYVSQPFLMDLEGKQGNFKNLREFVFKNIDGYFKIYSKHTPINL | |
| | | | VRDLPQGFSALEPLVDLPIGINITRFQTLLALHRSYLTPGDSSSGWTAGAAAYYVGYLQPRTFLLKYNE | |
| | | | NGTITDAVDCALDPLSETKCTLKSFTVEKGIYQTSNFRVQPTESIVRFPNITNLCPFGEVFNATRFASVY | |
| | | | AWNRKRISNCVADYSVLYNSASFSTFKCYGVSPTKLNDLCFTNVYADSFVIRGDEVRQIAPGQTGKIA | |
| | | | DYNYKLPDDFTGCVIAWNSNNLDSKVGGNYNYLYRLFRKSNLKPFERDISTEIYQAGSTPCNGVEGEN | |
| | | | CYFPLQSYGFQPTNGVGYQPYRVVVLSFELLHAPATVCGPKKSTNLVKNKCVNFNFNGLTGTGVLTE | |
| | | | SNKKFLPFQQFGRDIADTTDAVRDPQTLEILDITPCSFGGVSVITPGTNTSNQVAVLYQDVNCTEVPVAI | |
| | | | HADQLTPTWRVYSTGSNVFQTRAGCLIGAEHVNNSYECDIPIGAGICASYQTQTNSPGSASSVASQSII | |
| | | | AYTMSLGAENSVAYSNNSIAIPTNFTISVTTEILPVSMTKTSVDCTMYICGDSTECSNLLLQYGSFCTQL | |
| | | | NRALTGIAVEQDKNTQEVFAQVKQIYKTPPIKDFGGFNFSQILPDPSKPSKRSFIEDLLFNKVTLADAGFI | |
| | | | KQYGDCLGDIAARDLICAQKFNGLTVLPPLLTDEMIAQYTSALLAGTITSGWTFGAGAALQIPFAMQMA | |
| | | | YRFNGIGVTQNVLYENQKLIANQFNSAIGKIQDSLSSTASALGKLQDVVNQNAQALNTLVKQLSSNFGA | |
| | | | ISSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDVVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP577 | R-R(Omicron | MER5 (BatCoV-Ita1 206645- | QGVECDFSKLFKAAPPQIYNFSRLVFTNCNYNLTKLLSLFHVSEFSCHQVSPSALASGCYSSLTVDYFA | 214 |
| | BA4/5)-SpFN | 40) RBD_Omi-BA.4/5- | YPLYLASYLQQGSTGEIAQYNYKQDFSNPTCRILASVPANVSIPKPDKYIWLSQCYSFSAYSGDVPHYV | |
| | | RBD_SpFN | LPGQYTPCLYLTSSGFDNSYQTNRDFQNKMAATGVISSMTDNLQMAFVISVQYGTDTNSVCPMGSG | |
| | | | GSGNITNLCPFDEVFNATRFASVYAWNRKRISNCVADYSVLYNFAPFFAFKCYGVSPTKLNDLCFTNV | |
| | | | YADSFVIRGNEVSQIAPGQTGNIADYNYKLPDDFTGCVIAWNSNKLDSKVGGNYNYRYRLFRKSNLKP | |
| | | | FERDISTEIYQAGNKPCNGVAGVNCYFPLQSYGFRPTYGVGHQPYRVVVLSFELLHAPATVCGPGSG | |
| | | | GSGSSQCVNLTTRTQLPPAYTNSFTRGVYYPDKVFRSSVLHSTQDLFLPFFSNVTWFHAIHVSGTNGT | |
| | | | KRFDNPVLPFNDGVYFASTEKSNIIRGWIFGTTLDSKTQSLLIVNNATNWVIKVCEFQFCNDPFLGVYYH | |
| | | | KNNKSWMESEFRVYSSANNCTFEYVSQPFLMDLEGKQGNFKNLREFVFKNIDGYFKIYSKHTPINLVR | |
| | | | DLPQGFSALEPLVDLPIGINITRFQTLLALHRSYLTPGDSSSGWTAGAAAYYVGYLQPRTFLLKYNENG | |
| | | | TITDAVDCALDPLSETKCTLKSFTVEKGIYQTSNFRVQPTESIVRFPNITNLCPFGEVFNATRFASVYAW | |
| | | | NRKRISNCVADYSVLYNSASFSTFKCYGVSPTKLNDLCFTNVYADSFVIRGDEVRQIAPGQTGKIADYN | |
| | | | YKLPDDFTGCVIAWNSNNLDSKVGGNYNYLYRLFRKSNLKPFERDISTEIYQAGSTPCNGVEGFNCYF | |
| | | | PLQSYGFQPTNGVGYQPYRVVVLSFELLHAPATVCGPKKSTNLVKNKCVNFNFNGLTGTGVLTESNK | |
| | | | KFLPFQQFGRDIADTTDAVRDPQTLEILDITPCSFGGVSVITPGTNTSNQVAVLYQDVNCTEVPVAIHAD | |
| | | | QLTPTWRVYSTGSNVFQTRAGCLIGAEHVNNSYECDIPIGAGICASYQTQTNSPGSASSVASQSIIAYT | |
| | | | MSLGAENSVAYSNNSIAIPTNFTISVTTEILPVSMTKTSVDCTMYICGDSTECSNLLLQYGSFCTQLNRA | |
| | | | LTGIAVEQDKNTQEVFAQVKQIYKTPPIKDFGGFNFSQILPDPSKPSKRSFIEDLLFNKVTLADAGFIKQY | |
| | | | GDCLGDIAARDLICAQKFNGLTVLPPLLTDEMIAQYTSALLAGTITSGWTFGAGAALQIPFAMQMAYRF | |
| | | | NGIGVTQNVLYENQKLIANQFNSAIGKIQDSLSSTASALGKLQDVVNQNAQALNTLVKQLSSNFGAISS | |
| | | | VLNDILSRLDPPEAEVQIDRLITGRLQSLQTYVTQQLIRAAEIRASANLAATKMSECVLGQSKRVDFCGK | |
| | | | GYHLMSFPQSAPHGVVFLHVTYVPAQEKNFTTAPAICHDGKAHFPREGVFVSNGTHWFVTQRNFYEP | |
| | | | QIITTDNTFVSGNCDVVIGIVNNTVYDPLQSELDSIKEELDKIHKNGSGGSGDIIKLLNEQVNKEMQSSNL | |
| | | | YMSMSSWCYTHSLDGAGLFLFDHAAEEYEHAKKLIIFLNENNVPVQLTSISAPEHKFEGLTQIFQKAYE | |
| | | | HEQHISESINNIVDHAIKSKDHATFNFLQWYVAEQHEEEVLFKDILDKIELIGNENHGLYLADQYVKGIAK | |
| | | | SRKSGS | |
| pCoVP578 | R-R(Omicron | HKU5 RBD_Omi-BA.4/5- | TTQECDFTPMLTGTPPPIYNFKRLVFTNCNYNLTKLLSLFQVSEFSCHQVSPSSLATGCYSSLTVDYFA | 215 |
| | BA4/5)-SpFN | RBD_SpFN | YSTDMSSYLQPGSAGAIVQFNYKQDFSNPTCRVLATVPQNLTTITKPSNYAYLTECYKTSAYGKNYLY | |
| | | | NAPGAYTPCLSLASRGFSTKYQSHSDGELTTTGYIYPVTGNLQMAFIISVQYGTDTNSVCPMGSGGSG | |
| | | | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP579 | R-R(Omicron | SARS-COV-1 RBD_Omi-BA.4/5- | NITNLCPFGEVFNATKFPSVYAWERKKISNCVADYSVLYNSTFFSTFKCYGVSATKLNDLCFSNVYADS | 216 |
| | BA4/5)-SpFN | RBD_SpFN | FVVKGDDVRQIAPGQTGVIADYNYKLPDDFMGCVLAWNTRNIDATSTGNYNYKYRYLRHGKLRPFER | |
| | | | DISNVPFSPDGKPCTPPALNCYWPLNDYGFYTTTGIGYQPYRVVVLSFELLNAPATVCGPGSGGSGNI | |
| | | | TNLCPFDEVFNATRFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP580 | mosaic SpFN | SpFN with RBD from Omicron | SSQCVNLTTRTQLPPAYTNSFTRGVYYPDKVFRSSVLHSTQDLFLPFFSNVTWFHAIHVSGTNGTKRF | 217 |
| | | BA.4/5 replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP581 | mosaic SpFN | SpFN with RBD from Omicron | SSQCVNLTTRTQLPPAYTNSFTRGVYYPDKVFRSSVLHSTQDLFLPFFSNVTWFHAIHVSGTNGTKRF | 218 |
| | | BA.2.12.1 replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYQYRLFRKSNLKPFERD | |
| | | | ISTEIYQAGNKPCNGVAGFNCYFPLRSYGFRPTYGVGHQPYRVWVLSFELLHAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDWVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP582 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 219 |
| | | NC_014470.BM4831.BGR.2008 | DNPVLPFNDGVYFASTEKSNIIRGWIFGTTLDSKTQSLLIVNNATNVVIKVCEFQFCNDPFLGVYYHKNN | |
| | | replacing the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITQLCPFNEVFNITSFPSVYAWERMRITNCVADYSVLYNSSASFSTFQCYGVSPTKLNDLCFSSVYAD | |
| | | | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLASYGFTQSSGIGFQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP583 | mosaic SpFN | SpFN with BM48-31 RBD- | SSQCVNLTTRTQLPPAYTNSFTRGVYYPDKVFRSSVLHSTQDLFLPFFSNVTWFHAIHVSGTNGTKRF | 220 |
| | | T498W replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITQLCPFNEVFNITSFPSVYAWERMRITNCVADYSVLYNSSASFSTFQCYGVSPTKLNDLCFSSVYAD | |
| | | | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLASYGFWQSSGIGFQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP584 | mosaic SpFN | SpFN with BM48-31 RBD- | SSQCVNLTTRTQLPPAYTNSFTRGVYYPDKVFRSSVLHSTQDLFLPFFSNVTWFHAIHVSGTNGTKRF | 221 |
| | | A493Y-T498W replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITQLCPFNEVFNITSFPSVYAWERMRITNCVADYSVLYNSSASFSTFQCYGVSPTKLNDLCFSSVYAD | |
| | | | YFVVKGDDVRQIAPAQTGVIADYNYKLPDDFTGCVIAWNTNSLDSSNEFFYRRFRHGKIKPYGRDLSN | |
| | | | VLFNPSGGTCSAEGLNCYKPLYSYGFWQSSGIGFQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP585 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 222 |
| | | KC881005.RsSHC014 replacing | DNPVLPFNDGVYFASTEKSNIIRGWIFGTTLDSKTQSLLIVNNATNVVIKVCEFQFCNDPFLGVYYHKNN | |
| | | the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFGEVFNATTFPSVYAWERKRISNCVADYSVLYNSTSFSTFKCYGVSATKLNDLCFSNVYADS | |
| | | | FVVKGDDVRQIAPGQTGVIADYNYKLPDDFLGCVLAWNTNSKDSSTSGNYNYLYRWVRRSKLNPYER | |
| | | | DLSNDIYSPGGQSCSAVGPNCYNPLRPYGFFTTAGVGHQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDWNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP586 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 223 |
| | | KC881007.Bat.WIV1 replacing | DNPVLPFNDGVYFASTEKSNIIRGWIFGTTLDSKTQSLLIVNNATNVVIKVCEFQFCNDPFLGVYYHKNN | |
| | | the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFGEVFNATTFPSVYAWERKRISNCVADYSVLYNSTSFSTFKCYGVSATKLNDLCFSNVYADS | |
| | | | FVVKGDDVRQIAPGQTGVIADYNYKLPDDFTGCVLAWNTRNIDATQTGNYNYKYRSLRHGKLRPFER | |
| | | | DISNVPFSPDGKPCTPPAFNCYWPLNDYGFYITNGIGYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDWVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP587 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 224 |
| | | AVP78042_ | DNPVLPFNDGVYFASTEKSNIIRGWIFGTTLDSKTQSLLIVNNATNVVIKVCEFQFCNDPFLGVYYHKNN | |
| | | BatSARSlikecoronavirus | KSWMESEFRVYSSANNCTFEYVSQPFLMDLEGKQGNFKNLREFVFKNIDGYFKIYSKHTPINLVRDLP | |
| | | replacing the original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNVCPFHKVFNATRFPSVYAWERTKISDCIADYTVFYNSTSFSTFKCYGVSPSKLIDLCFTSVYADTF | |
| | | | LIRFSEVRQVAPGQTGVIADYNYKLPDDFTGCVIAWNTAKQDTGHYFYRSHRSTKLKPFERDLSSDEN | |
| | | | GVRTLSTYDFNPNVPLEYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP588 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 225 |
| | | MZ937001_BANAL20103_Laos_ | DNPVLPFNDGVYFASTEKSNIIRGWIFGTTLDSKTQSLLIVNNATNVVIKVCEFQFCNDPFLGVYYHKNN | |
| | | 2020 replacing the | KSWMESEFRVYSSANNCTFEYVSQPFLMDLEGKQGNFKNLREFVFKNIDGYFKIYSKHTPINLVRDLP | |
| | | original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFGEVFNATTFASVYAWNRKRISNCVADYSVLYNSTSFSTFKCYGVSPTKLNDLCFTNVYADS | |
| | | | FVVRGDEVRQIAPGQTGKIADYNYKLPDDFTGCVIAWNSNNLDSKVGGNYNYLYRLFRKSNLKPFERD | |
| | | | ISTEIYQAGSTPCNGVEGFNCYFPLQSYGFHPTNGVGYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP589 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 226 |
| | | MN996532.2013.Bat.RaTG13 | DNPVLPFNDGVYFASTEKSNIIRGWIFGTTLDSKTQSLLIVNNATNVVIKVCEFQFCNDPFLGVYYHKNN | |
| | | replacing the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFGEVFNATTFASVYAWNRKRISNCVADYSVLYNSTSFSTFKCYGVSPTKLNDLCFTNVYADS | |
| | | | FVITGDEVRQIAPGQTGKIADYNYKLPDDFTGCVIAWNSKHIDAKEGGNFNYLYRLFRKANLKPFERDI | |
| | | | STEIYQAGSKPCNGQTGLNCYYPLYRYGFYPTDGVGHQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP590 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 227 |
| | | MZ937004_BANAL20247_ | DNPVLPFNDGVYFASTEKSNIIRGWIFGTTLDSKTQSLLIVNNATNVVIKVCEFQFCNDPFLGVYYHKNN | |
| | | Laos_2020 replacing the | KSWMESEFRVYSSANNCTFEYVSQPFLMDLEGKQGNFKNLREFVFKNIDGYFKIYSKHTPINLVRDLP | |
| | | original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNFCPFDKVFNATRFPNVYAWQRTKISDCIADYTVLYNSTSFSTFKCYGVSPSKLIDLCFTSVYADTF | |
| | | | LIRFSEVRQIAPGETGVIADYNYKLPDDFTGCVLAWNTAQQDIGSYFYRSHRAVKLKPFERDLSSDEN | |
| | | | GVRTLSTYDFNPNVPLDYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP591 | mosaic SpFN | SpFN with BANAL-20-247 | SSQCVNLTTRTQLPPAYTNSFTRGVYYPDKVFRSSVLHSTQDLFLPFFSNVTWFHAIHVSGTNGTKRF | 228 |
| | | RBD-N498W replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNFCPFDKVFNATRFPNVYAWQRTKISDCIADYTVLYNSTSFSTFKCYGVSPSKLIDLCFTSVYADTF | |
| | | | LIRFSEVRQIAPGETGVIADYNYKLPDDFTGCVLAWNTAQQDIGSYFYRSHRAVKLKPFERDLSSDEN | |
| | | | GVRTLSTYDFWPNVPLDYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP592 | mosaic SpFN | SpFN with BANAL-20-247 | SSQCVNLTTRTQLPPAYTNSFTRGVYYPDKVFRSSVLHSTQDLFLPFFSNVTWFHAIHVSGTNGTKRF | 229 |
| | | RBD-S493Y-N498W replacing | DNPVLPFNDGVYFASTEKSNIIRGWIFGTTLDSKTQSLLIVNNATNVVIKVCEFQFCNDPFLGVYYHKNN | |
| | | the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNFCPFDKVFNATRFPNVYAWQRTKISDCIADYTVLYNSTSFSTFKCYGVSPSKLIDLCFTSVYADTF | |
| | | | LIRFSEVRQIAPGETGVIADYNYKLPDDFTGCVLAWNTAQQDIGSYFYRSHRAVKLKPFERDLSSDEN | |
| | | | GVRTLYTYDFWPNVPLDYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP593 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 230 |
| | | ATO98145_ | DNPVLPFNDGVYFASTEKSNIIRGWIFGTTLDSKTQSLLIVNNATNWVIKVCEFQFCNDPFLGVYYHKNN | |
| | | BatSARSlikecoronavirus | KSWMESEFRVYSSANNCTFEYVSQPFLMDLEGKQGNFKNLREFVFKNIDGYFKIYSKHTPINLVRDLP | |
| | | replacing the original | QGFSALEPLVDLPIGINITRFQTLLALHRSYLTPGDSSSGWTAGAAAYYVGYLQPRTFLLKYNENGTITD | |
| | | RBD | AVDCALDPLSETKCTLKSFTVEKGIYQTSNFRVQPTESIVRFP | |
| | | | NITNLCPFDAVFNATRFPSVYAWERVKISNCVADYTAFYNSTSFSTFKCYGVSPSKLIDLCFTSVYADT | |
| | | | FLIRFSEVRQVAPGETGVIADYNYKLPDDFTGCVIAWNTAKQDVGSYFYRSHRSSKLKPFERDLSSDE | |
| | | | NGVRTLSTYDFNPNVPLDYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP594 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 231 |
| | | JX993987.2011.BatRp. | DNPVLPFNDGVYFASTEKSNIIRGWIFGTTLDSKTQSLLIVNNATNVVIKVCEFQFCNDPFLGVYYHKNN | |
| | | Shaanxi2011 | KSWMESEFRVYSSANNCTFEYVSQPFLMDLEGKQGNFKNLREFVFKNIDGYFKIYSKHTPINLVRDLP | |
| | | replacing the original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNRCPFDKVFNATRFPSVYAWERTKISDCVADYTVLYNSTSFSTFKCYGVSPSKLIDLCFTSVYADT | |
| | | | FLIRSSEVRQVAPGETGVIADYNYKLPDDFTGCVIAWNTANQDQGQYYYRSSRKEKLKPFERDLSSDE | |
| | | | NGVYTLSTYDFYPSVPLDYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP595 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 232 |
| | | KJ473812.BtRfBetaCoV. | DNPVLPFNDGVYFASTEKSNIIRGWIFGTTLDSKTQSLLIVNNATNVVIKVCEFQFCNDPFLGVYYHKNN | |
| | | HeB2013 | KSWMESEFRVYSSANNCTFEYVSQPFLMDLEGKQGNFKNLREFVFKNIDGYFKIYSKHTPINLVRDLP | |
| | | replacing the original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFDKVFNATRFPSVYAWERTKISDCVADYTVFYNSTSFSTFNCYGVSPSKLIDLCFTSVYADT | |
| | | | FLIRFSEVRQVAPGQTGVIADYNYKLPDDFTGCVIAWNTAKQDVGSYFYRSHRSSKLKPFERDLSSEE | |
| | | | NGVRTLSTYDFNQYVPLEYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP596 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 233 |
| | | DQ071615.2003.Bat.Rp3 | DNPVLPFNDGVYFASTEKSNIIRGWIFGTTLDSKTQSLLIVNNATNVVIKVCEFQFCNDPFLGVYYHKNN | |
| | | replacing | KSWMESEFRVYSSANNCTFEYVSQPFLMDLEGKQGNFKNLREFVFKNIDGYFKIYSKHTPINLVRDLP | |
| | | the original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNRCPFDKVFNATRFPNVYAWERTKISDCVADYTVLYNSTSFSTFKCYGVSPSKLIDLCFTSVYADT | |
| | | | FLIRSSEVRQVAPGETGVIADYNYKLPDDFTGCVIAWNTAKQDQGQYYYRSHRKTKLKPFERDLSSDE | |
| | | | NGVRTLSTYDFYPSVPVAYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP597 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 234 |
| | | ACU31032_Rs_672_2006 | DNPVLPFNDGVYFASTEKSNIIRGWIFGTTLDSKTQSLLIVNNATNVVIKVCEFQFCNDPFLGVYYHKNN | |
| | | replacing | KSWMESEFRVYSSANNCTFEYVSQPFLMDLEGKQGNFKNLREFVFKNIDGYFKIYSKHTPINLVRDLP | |
| | | the original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNRCPFDKVFNASRFPNVYAWERTKISDCVADYTVLYNSTSFSTFKCYGVSPSKLIDLCFTSVYADT | |
| | | | FLIRSSEVRQVAPGETGVIADYNYKLPDDFTGCVIAWNTAKQDQGQYYYRSSRKTKLKPFERDLTSDE | |
| | | | NGVRTLSTYDFYPNVPIEYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP598 | mosaic SpFN | SpFN with RBD from | SSQCVNLTTRTQLPPAYTNSFTRGVYYPDKVFRSSVLHSTQDLFLPFFSNVTWFHAIHVSGTNGTKRF | 235 |
| | | MZ081380_bat_Yunnan_ | DNPVLPFNDGVYFASTEKSNIIRGWIFGTTLDSKTQSLLIVNNATNVVIKVCEFQFCNDPFLGVYYHKNN | |
| | | RsYN04_2020 replacing the | KSWMESEFRVYSSANNCTFEYVSQPFLMDLEGKQGNFKNLREFVFKNIDGYFKIYSKHTPINLVRDLP | |
| | | original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFSQVFNATRFPSVYAWTRERISNCIADYSVLYNSTSFSTFRCYGVSPTKLNDLCFSNVYADS | |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFTSSVSQDYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP599 | mosaic SpFN | SpFN with RsYN04 RBD-T498W | SSQCVNLTTRTQLPPAYTNSFTRGVYYPDKVFRSSVLHSTQDLFLPFFSNVTWFHAIHVSGTNGTKRF | 236 |
| | | replacing the original | DNPVLPFNDGVYFASTEKSNIIRGWIFGTTLDSKTQSLLIVNNATNVVIKVCEFQFCNDPFLGVYYHKNN | |
| | | RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFSQVFNATRFPSVYAWTRERISNCIADYSVLYNSTSFSTFRCYGVSPTKLNDLCFSNVYADS | |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFWSSVSQDYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP600 | mosaic SpFN | SpFN with RsYN04 RBD- | SSQCVNLTTRTQLPPAYTNSFTRGVYYPDKVFRSSVLHSTQDLFLPFFSNVTWFHAIHVSGTNGTKRF | 237 |
| | | L493Y-T498W replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFSQVFNATRFPSVYAWTRERISNCIADYSVLYNSTSFSTFRCYGVSPTKLNDLCFSNVYADS | |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLYKYGFWSSVSQDYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP601 | mosaic SpFN | SpFN with Khosta2-MZ190138 | SSQCVNLTTRTQLPPAYTNSFTRGVYYPDKVFRSSVLHSTQDLFLPFFSNVTWFHAIHVSGTNGTKRF | 238 |
| | | replacing the original RBD | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NMTNICPFDQVFNKTQFPSVYAWERVRISDCVSDYTVLYNSSASFSTFKCYGVSPTKLNDLCFSGVYA | |
| | | | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFTSTSGVGYQPFRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP602 | mosaic SpFN | SpFN with Khosta-2 RBD- | SSQCVNLTTRTQLPPAYTNSFTRGVYYPDKVFRSSVLHSTQDLFLPFFSNVTWFHAIHVSGTNGTKRF | 239 |
| | | T498W replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NMTNICPFDQVFNKTQFPSVYAWERVRISDCVSDYTVLYNSSASFSTFKCYGVSPTKLNDLCFSGVYA | |
| | | | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLQDYGFWSTSGVGYQPFRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP603 | mosaic SpFN | SpFN with Khosta-2 RBD- | SSQCVNLTTRTQLPPAYTNSFTRGVYYPDKVFRSSVLHSTQDLFLPFFSNVTWFHAIHVSGTNGTKRF | 240 |
| | | Q493Y-T498W replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NMTNICPFDQVFNKTQFPSVYAWERVRISDCVSDYTVLYNSSASFSTFKCYGVSPTKLNDLCFSGVYA | |
| | | | DYFWVKGDHVHQIAPGQTGVIADYNYKLPSEFVGCILAWNTRTIDSKRGFYYRLFRHGNIRPYERDTSN | |
| | | | VPYNAAGGTCNQPGTHNCYEPLYDYGFWSTSGVGYQPFRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP604 | mosaic SpFN | SpFN with KY352407_BtKY72_ | SSQCVNLTTRTQLPPAYTNSFTRGVYYPDKVFRSSVLHSTQDLFLPFFSNVTWFHAIHVSGTNGTKRF | 241 |
| | | bat_Kenya replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFGQVFNASNFPSVYAWERLRISDCVADYAVLYNSSSSFSTFKCYGVSPTKLNDLCFSSVYA | |
| | | | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFTPTVGVGYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP605 | mosaic SpFN | SpFN with BtKY72 RBD-T498W | SSQCVNLTTRTQLPPAYTNSFTRGVYYPDKVFRSSVLHSTQDLFLPFFSNVTWFHAIHVSGTNGTKRF | 242 |
| | | replacing the original | DNPVLPFNDGVYFASTEKSNIIRGWIFGTTLDSKTQSLLIVNNATNVVIKVCEFQFCNDPFLGVYYHKNN | |
| | | RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFGQVFNASNFPSVYAWERLRISDCVADYAVLYNSSSSFSTFKCYGVSPTKLNDLCFSSVYA | |
| | | | DYFWVKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLKSYGFWPTVGVGYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP606 | mosaic SpFN | SpFN with BtKY72 RBD- | SSQCVNLTTRTQLPPAYTNSFTRGVYYPDKVFRSSVLHSTQDLFLPFFSNVTWFHAIHVSGTNGTKRF | 243 |
| | | K493Y-T498W replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFGQVFNASNFPSVYAWERLRISDCVADYAVLYNSSSSFSTFKCYGVSPTKLNDLCFSSVYA | |
| | | | DYFWKGDDVRQIAPAQTGVIADYNYKLPDDFTGCVLAWNTNSVDSKSGNNFYYRLFRHGKIKPYER | |
| | | | DISNVLYNSAGGTCSSISQLGCYEPLYSYGFWPTVGVGYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP607 | mosaic SpFN | SpFN with RaTG15_Ra7909 | SSQCVNLTTRTQLPPAYTNSFTRGVYYPDKVFRSSVLHSTQDLFLPFFSNVTWFHAIHVSGTNGTKRF | 244 |
| | | (RBD) replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNLCPFSQVFNATRFPSVYAWTRERISNCIADYSVLYNSTSFSTFRCYGVSPTKLNDLCFSNVYADS | |
| | | | MVVRGDEVRQIAPSQTGVIADYNYKLPDDFTGCVIAWNSKAKDENGQYFYRLFRKSKLLPFQRDVSN | |
| | | | VTYGSGKNDGCNPSEADCYWPLLKYGFTGSVSQDYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDWNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP608 | mosaic SpFN | SpFN with HKU3- | SSQCVNLTTRTQLPPAYTNSFTRGVYYPDKVFRSSVLHSTQDLFLPFFSNVTWFHAIHVSGTNGTKRF | 245 |
| | | 1_DQ022305.2005 (RBD) | DNPVLPFNDGVYFASTEKSNIIRGWIFGTTLDSKTQSLLIVNNATNVVIKVCEFQFCNDPFLGVYYHKNN | |
| | | replacing the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NITNRCPFDKVFNATRFPNVYAWERTKISDCVADYTVLYNSTSFSTFKCYGVSPSKLIDLCFTSVYADT | |
| | | | FLIRSSEVRQVAPGETGVIADYNYKLPDDFTGCVIAWNTAKHDTGNYYYRSHRKTKLKPFERDLSSDD | |
| | | | GNGVYTLSTYDFNPNVPVAYQATRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP609 | mosaic SpFN | SpFN with RhGB01-MW719567 | SSQCVNLTTRTQLPPAYTNSFTRGVYYPDKVFRSSVLHSTQDLFLPFFSNVTWFHAIHVSGTNGTKRF | 246 |
| | | replacing the original | DNPVLPFNDGVYFASTEKSNIIRGWIFGTTLDSKTQSLLIVNNATNVVIKVCEFQFCNDPFLGVYYHKNN | |
| | | RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | NVTQLCPFSDVFNRTMFPSVYAWERMSIRDCVADYAVLYNSSISFSTFECYGVSPTKLNDLCFSSVYA | |
| | | | DYFWVRGDDVKQIAPGKTGVIADYNYKLPDDFVGCVLAWNSRHVDNKQGFYYRLFRHGKIKPYQRDM | |
| | | | SRELYSPSGTPCTWVGFNCYDPLAHYAFASPGIGIGYQPFRVVVLSFQLLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP610 | mosaic SpFN | SpFN with | SSQCVNLTTRTQLPPAYTNSFTRGVYYPDKVFRSSVLHSTQDLFLPFFSNVTWFHAIHVSGTNGTKRF | 247 |
| | | bioinformatically | DNPVLPFNDGVYFASTEKSNIIRGWIFGTTLDSKTQSLLIVNNATNVVIKVCEFQFCNDPFLGVYYHKNN | |
| | | derived Cluster1_RBD (SARS | KSWMESEFRVYSSANNCTFEYVSQPFLMDLEGKQGNFKNLREFVFKNIDGYFKIYSKHTPINLVRDLP | |
| | | related) replacing the | QGFSALEPLVDLPIGINITRFQTLLALHRSYLTPGDSSSGWTAGAAAYYVGYLQPRTFLLKYNENGTITD | |
| | | original RBD | AVDCALDPLSETKCTLKSFTVEKGIYQTSNFRVQPTESIVRFP | |
| | | | NITNLCPFDEVFNATRFPSVYAWERKKISNCVADYSVLYNSTSFSTFKCYGVSPSKLNDLCFTSVYADS | |
| | | | FVIRGDEVRQIAPGQTGVIADYNYKLPDDFTGCVIAWNTANQDSGEGGNPNYYYRSSRKSKLKPFER | |
| | | | DLSSDEYNVGSQPCNRMGPNCYRTLSTYDFYPTVGVGYQPYRVVVLSFELLNAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP611 | mosaic SpFN | SpFN with | SSQCVNLTTRTQLPPAYTNSFTRGVYYPDKVFRSSVLHSTQDLFLPFFSNVTWFHAIHVSGTNGTKRF | 248 |
| | | bioinformatically derived | DNPVLPFNDGVYFASTEKSNIIRGWIFGTTLDSKTQSLLIVNNATNVVIKVCEFQFCNDPFLGVYYHKNN | |
| | | Cluster2_RBD (QC43- | KSWMESEFRVYSSANNCTFEYVSQPFLMDLEGKQGNFKNLREFVFKNIDGYFKIYSKHTPINLVRDLP | |
| | | related) replacing the | QGFSALEPLVDLPIGINITRFQTLLALHRSYLTPGDSSSGWTAGAAAYYVGYLQPRTFLLKYNENGTITD | |
| | | original RBD | AVDCALDPLSETKCTLKSFTVEKGIYQTSNFRVQPTESIVRFP | |
| | | | NLPDCNIEEWLTAKSVPSPLNWERKTFSNCNFNLSSLLRFVQADSFSCNNIDASKVYGMCFGSISVDK | |
| | | | FAIPNSRQVDLQLGNSGFLQSFNYKIDTTATSCQLYYSLPANNVTINNYNPSSWNRRYGFNDDTVFKP | |
| | | | RPAGVLTNHDVVYSQQCFKVPNTFCPCKNNGSCVSSGKGTTNGSGTCPAGTNYRTCENTTWCACS | |
| | | | CTPDPITASGPRKCPQKKSLVGVGEHCSGLGVDSDYCGNVSCLCSTDAFLGWSADSCLVNDRCNIFA | |
| | | | NIILNGINSGTTCST | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP612 | mosaic SpFN | SpFN with | SSQCVNLTTRTQLPPAYTNSFTRGVYYPDKVFRSSVLHSTQDLFLPFFSNVTWFHAIHVSGTNGTKRF | 249 |
| | | bioinformatically derived | DNPVLPFNDGVYFASTEKSNIIRGWIFGTTLDSKTQSLLIVNNATNVVIKVCEFQFCNDPFLGVYYHKNN | |
| | | Cluster3_RBD | KSWMESEFRVYSSANNCTFEYVSQPFLMDLEGKQGNFKNLREFVFKNIDGYFKIYSKHTPINLVRDLP | |
| | | (MERS-related) replacing | QGFSALEPLVDLPIGINITRFQTLLALHRSYLTPGDSSSGWTAGAAAYYVGYLQPRTFLLKYNENGTITD | |
| | | the original RBD | AVDCALDPLSETKCTLKSFTVEKGIYQTSNFRVQPTESIVRFP | |
| | | | QGVECDFSKLFKAAAPQIYNFSRLVFTNCNYNLTKLLSLFQVDEFSCHQISPSALASGCYSSLTVDYFA | |
| | | | YPLSMKSYLQIGSAGEISQFNYKQDFSNPTCRILASVPANVSITKPDKYIYISQCYSLSAYSDVDVPHYV | |
| | | | NPGQYTSIPCLYLTSSGFDEDGQYFRYNLTQFEGQNYLVATGVTVSMTDNLQMVFVITVQYGTDTNS | |
| | | | VCP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDVVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP613 | mosaic SpFN | SpFN with MER1 | SSQCVNLTTRTQLPPAYTNSFTRGVYYPDKVFRSSVLHSTQDLFLPFFSNVTWFHAIHVSGTNGTKRF | 250 |
| | | (ErinaceusCoV/2012- | DNPVLPFNDGVYFASTEKSNIIRGWIFGTTLDSKTQSLLIVNNATNVVIKVCEFQFCNDPFLGVYYHKNN | |
| | | 174/GER/2012) | KSWMESEFRVYSSANNCTFEYVSQPFLMDLEGKQGNFKNLREFVFKNIDGYFKIYSKHTPINLVRDLP | |
| | | replacing the original RBD | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | ELTECDLDVLFKNDAPIIANYSRRVFTNCNYNLTKLLSLVQVDEFVCHKTTPEALATGCYSSLTVDWFA | |
| | | | LPFSMKSTLAIGSAEAISMFNYNQDYSNPTCRIHAAVTANVSTALNFTANANYAYISRCQGVDGKPILLQ | |
| | | | PGQMTNIACRSGVLARPSDADYFGYSFQGRNYYLGRKSYKPKTDEGDVQMVYVITPKYDKGPDTVC | |
| | | | PLK | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP614 | mosaic SpFN | SpFN with MER2 (Neoromicia/ | SSQCVNLTTRTQLPPAYTNSFTRGVYYPDKVFRSSVLHSTQDLFLPFFSNVTWFHAIHVSGTNGTKRF | 251 |
| | | 5038) replacing the | DNPVLPFNDGVYFASTEKSNIIRGWIFGTTLDSKTQSLLIVNNATNVVIKVCEFQFCNDPFLGVYYHKNN | |
| | | original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | VYPDCNFTELFRERAPTIMQYKREVFTRCNYNLSLLLSLVQVDEFVCDKITPEALATGCYSSLTVDWFA | |
| | | | FPYAWKSYLAIGSADRIVRFNYNQDYSNPSCRIHSKVNSSIGISYAGAYSYITNCNYGATNKDDVVKPG | |
| | | | GRASQQCITGALNSPTTGQLWAYNFGGVPYRVSRLTYTDHLSDPLDMVYVITVKYEPGAETVCPKQ | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP615 | mosaic SpFN | SpFN with MER3 (HKU4 SM3A) | SSQCVNLTTRTQLPPAYTNSFTRGVYYPDKVFRSSVLHSTQDLFLPFFSNVTWFHAIHVSGTNGTKRF | 252 |
| | | replacing the original RBD | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | QATECDFSPMLTGVAPQVYNFKRLVFSNCNYNLTKLLSLFAVDEFSCNGISPDAIARGCYSTLTVDYFA | |
| | | | YPLSMKSYIRPGSAGNIPLYNYKQSFANPTCRVMASVPANVTITKPDAYGYISKCSRLTGANQDVETPL | |
| | | | YINPGEYSICRDFSPGGFSEDGQVFKRTLTQFEGGGLLIGVGTRVPMTDNLQMSFIIFVQYGTGTDSV | |
| | | | CPML | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP616 | mosaic SpFN | SpFN with MER4 (BatCoV- | SSQCVNLTTRTQLPPAYTNSFTRGVYYPDKVFRSSVLHSTQDLFLPFFSNVTWFHAIHVSGTNGTKRF | 253 |
| | | Ita2 206645-63) replacing | DNPVLPFNDGVYFASTEKSNIIRGWIFGTTLDSKTQSLLIVNNATNVVIKVCEFQFCNDPFLGVYYHKNN | |
| | | the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | QGVECDFSKLFKAAPPQIYNFSRLVFTNCNYNLTKLLSLFHVSEFSCHQVSPSALASGCYSSLTVDYFA | |
| | | | YPLYLASYLQQGSTGEIAQYNYKQDFSNPTCRILASVPANVSIPKPDKYIWLSQCYSFSAYSGDIPHYV | |
| | | | QPGQYTPCLYLTSSGFDKSYQTNRDMQNKMAATGVTSSMTDNLQMAFVISVQYGTDTNSVCPM | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP617 | mosaic SpFN | SpFN with MER5 (BatCoV- | SSQCVNLTTRTQLPPAYTNSFTRGVYYPDKVFRSSVLHSTQDLFLPFFSNVTWFHAIHVSGTNGTKRF | 254 |
| | | Ita1 206645-40) replacing | DNPVLPFNDGVYFASTEKSNIIRGWIFGTTLDSKTQSLLIVNNATNVVIKVCEFQFCNDPFLGVYYHKNN | |
| | | the original RBD | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | QGVECDFSKLFKAAPPQIYNFSRLVFTNCNYNLTKLLSLFHVSEFSCHQVSPSALASGCYSSLTVDYFA | |
| | | | YPLYLASYLQQGSTGEIAQYNYKQDFSNPTCRILASVPANVSIPKPDKYIWLSQCYSFSAYSGDVPHYV | |
| | | | LPGQYTPCLYLTSSGFDNSYQTNRDFQNKMAATGVISSMTDNLQMAFVISVQYGTDTNSVCPM | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP618 | mosaic SpFN | SpFN with HKU5 replacing | SSQCVNLTTRTQLPPAYTNSFTRGVYYPDKVFRSSVLHSTQDLFLPFFSNVTWFHAIHVSGTNGTKRF | 255 |
| | | the original RBD | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFP | |
| | | | TTQECDFTPMLTGTPPPIYNFKRLVFTNCNYNLTKLLSLFQVSEFSCHQVSPSSLATGCYSSLTVDYFA | |
| | | | YSTDMSSYLQPGSAGAIVQFNYKQDFSNPTCRVLATVPQNLTTITKPSNYAYLTECYKTSAYGKNYLY | |
| | | | NAPGAYTPCLSLASRGFSTKYQSHSDGELTTTGYIYPVTGNLQMAFIISVQYGTDTNSVCPM | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| pCoVP619 | (R)-mosaic-SpFN | RBD from Omicron BA.4/5_ | NITNLCPFDEVFNATRFASVYAWNRKRISNCVADYSVLYNFAPFFAFKCYGVSPTKLNDLCFTNVYADS | 256 |
| | | mosaicSpFN with SARS-1 | FVIRGNEVSQIAPGQTGNIADYNYKLPDDFTGCVIAWNSNKLDSKVGGNYNYRYRLFRKSNLKPFERDI | |
| | | replacing the original RBD | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP620 | (R)-mosaic-SpFN | RBD from Omicron BA.2.12. | NITNLCPFDEVFNATRFASVYAWNRKRISNCVADYSVLYNFAPFFAFKCYGVSPTKLNDLCFTNVYADS | 257 |
| | | 1_mosaicSpFN with SARS-1 | FVIRGNEVSQIAPGQTGNIADYNYKLPDDFTGCVIAWNSNKLDSKVGGNYNYQYRLFRKSNLKPFERD | |
| | | replacing the original RBD | ISTEIYQAGNKPCNGVAGFNCYFPLRSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP621 | (R)-mosaic-SpFN | RBD from NC_014470.BM4831. | NITQLCPFNEVFNITSFPSVYAWERMRITNCVADYSVLYNSSASFSTFQCYGVSPTKLNDLCFSSVYAD | 258 |
| | | BGR.2008_mosaicSpFN | YFVVKGDDVRQIAPAQTGVIADYNYKLPDDFTGCVIAWNTNSLDSSNEFFYRRFRHGKIKPYGRDLSN | |
| | | with SARS-1 replacing the | VLFNPSGGTCSAEGLNCYKPLASYGFTQSSGIGFQPYRVVVLSFELLNAPATVCGPGSGGSGSSQCV | |
| | | original RBD | NLTTRTQLPPAYTNSFTRGVYYPDKVFRSSVLHSTQDLFLPFFSNVTWFHAIHVSGTNGTKRFDNPVL | |
| | | | PFNDGVYFASTEKSNIIRGWIFGTTLDSKTQSLLIVNNATNVVIKVCEFQFCNDPFLGVYYHKNNKSWM | |
| | | | ESEFRVYSSANNCTFEYVSQPFLMDLEGKQGNFKNLREFVFKNIDGYFKIYSKHTPINLVRDLPQGFSA | |
| | | | LEPLVDLPIGINITRFQTLLALHRSYLTPGDSSSGWTAGAAAYYVGYLQPRTFLLKYNENGTITDAVDCA | |
| | | | LDPLSETKCTLKSFTVEKGIYQTSNFRVQPTESIVRFPNITNLCPFGEVFNATKFPSVYAWERKKISNCV | |
| | | | ADYSVLYNSTFFSTFKCYGVSATKLNDLCFSNVYADSFVVKGDDVRQIAPGQTGVIADYNYKLPDDFM | |
| | | | GCVLAWNTRNIDATSTGNYNYKYRYLRHGKLRPFERDISNVPFSPDGKPCTPPALNCYWPLNDYGFY | |
| | | | TTTGIGYQPYRVVVLSFELLNAPATVCGPKKSTNLVKNKCVNFNFNGLTGTGVLTESNKKFLPFQQFG | |
| | | | RDIADTTDAVRDPQTLEILDITPCSFGGVSVITPGTNTSNQVAVLYQDVNCTEVPVAIHADQLTPTWRV | |
| | | | YSTGSNVFQTRAGCLIGAEHVNNSYECDIPIGAGICASYQTQTNSPGSASSVASQSIIAYTMSLGAENS | |
| | | | VAYSNNSIAIPTNFTISVTTEILPVSMTKTSVDCTMYICGDSTECSNLLLQYGSFCTQLNRALTGIAVEQD | |
| | | | KNTQEVFAQVKQIYKTPPIKDFGGFNFSQILPDPSKPSKRSFIEDLLFNKVTLADAGFIKQYGDCLGDIA | |
| | | | ARDLICAQKFNGLTVLPPLLTDEMIAQYTSALLAGTITSGWTFGAGAALQIPFAMQMAYRFNGIGVTQN | |
| | | | VLYENQKLIANQFNSAIGKIQDSLSSTASALGKLQDVVNQNAQALNTLVKQLSSNFGAISSVLNDILSRL | |
| | | | DPPEAEVQIDRLITGRLQSLQTYVTQQLIRAAEIRASANLAATKMSECVLGQSKRVDFCGKGYHLMSFP | |
| | | | QSAPHGVVFLHVTYVPAQEKNFTTAPAICHDGKAHFPREGVFVSNGTHWFVTQRNFYEPQIITTDNTF | |
| | | | VSGNCDWVIGIVNNTVYDPLQSELDSIKEELDKIHKNGSGGSGDIIKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESI | |
| | | | NNIVDHAIKSKDHATFNFLQWYVAEQHEEEVLFKDILDKIELIGNENHGLYLADQYVKGIAKSRKSGS | |
| pCoVP622 | (R)-mosaic-SpFN | BM48-31 RBD-T498W_ | NITQLCPFNEVFNITSFPSVYAWERMRITNCVADYSVLYNSSASFSTFQCYGVSPTKLNDLCFSSVYAD | 259 |
| | | mosaicSpFN with SARS-1 | YFVVKGDDVRQIAPAQTGVIADYNYKLPDDFTGCVIAWNTNSLDSSNEFFYRRFRHGKIKPYGRDLSN | |
| | | replacing the original RBD | VLFNPSGGTCSAEGLNCYKPLASYGFWQSSGIGFQPYRVVVLSFELLNAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATKFPSVYAWERKKISN | |
| | | | CVADYSVLYNSTFFSTFKCYGVSATKLNDLCFSNVYADSFVVKGDDVRQIAPGQTGVIADYNYKLPDD | |
| | | | FMGCVLAWNTRNIDATSTGNYNYKYRYLRHGKLRPFERDISNVPFSPDGKPCTPPALNCYWPLNDYG | |
| | | | FYTTTGIGYQPYRVWVLSFELLNAPATVCGPKKSTNLVKNKCVNFNFNGLTGTGVLTESNKKFLPFQQ | |
| | | | FGRDIADTTDAVRDPQTLEILDITPCSFGGVSVITPGTNTSNQVAVLYQDVNCTEVPVAIHADQLTPTW | |
| | | | RVYSTGSNVFQTRAGCLIGAEHVNNSYECDIPIGAGICASYQTQTNSPGSASSVASQSIIAYTMSLGAE | |
| | | | NSVAYSNNSIAIPTNFTISVTTEILPVSMTKTSVDCTMYICGDSTECSNLLLQYGSFCTQLNRALTGIAVE | |
| | | | QDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP623 | (R)-mosaic-SpFN | BM48-31 RBD-A493Y-T498W_ | NITQLCPFNEVFNITSFPSVYAWERMRITNCVADYSVLYNSSASFSTFQCYGVSPTKLNDLCFSSVYAD | 260 |
| | | mosaicSpFN with SARS-1 | YFVVKGDDVRQIAPAQTGVIADYNYKLPDDFTGCVIAWNTNSLDSSNEFFYRRFRHGKIKPYGRDLSN | |
| | | replacing the original RBD | VLFNPSGGTCSAEGLNCYKPLYSYGFWQSSGIGFQPYRVVVLSFELLNAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATKFPSVYAWERKKISN | |
| | | | CVADYSVLYNSTFFSTFKCYGVSATKLNDLCFSNVYADSFVVKGDDVRQIAPGQTGVIADYNYKLPDD | |
| | | | FMGCVLAWNTRNIDATSTGNYNYKYRYLRHGKLRPFERDISNVPFSPDGKPCTPPALNCYWPLNDYG | |
| | | | FYTTTGIGYQPYRVVVLSFELLNAPATVCGPKKSTNLVKNKCVNFNFNGLTGTGVLTESNKKFLPFQQ | |
| | | | FGRDIADTTDAVRDPQTLEILDITPCSFGGVSVITPGTNTSNQVAVLYQDVNCTEVPVAIHADQLTPTW | |
| | | | RVYSTGSNVFQTRAGCLIGAEHVNNSYECDIPIGAGICASYQTQTNSPGSASSVASQSIIAYTMSLGAE | |
| | | | NSVAYSNNSIAIPTNFTISVTTEILPVSMTKTSVDCTMYICGDSTECSNLLLQYGSFCTQLNRALTGIAVE | |
| | | | QDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP624 | (R)-mosaic-SpFN | RBD from KC881005. | NITNLCPFGEVFNATTFPSVYAWERKRISNCVADYSVLYNSTSFSTFKCYGVSATKLNDLCFSNVYADS | 261 |
| | | RsSHC014 _mosaicSpFN with | FVVKGDDVRQIAPGQTGVIADYNYKLPDDFLGCVLAWNTNSKDSSTSGNYNYLYRWVRRSKLNPYER | |
| | | SARS-1 replacing the | DLSNDIYSPGGQSCSAVGPNCYNPLRPYGFFTTAGVGHQPYRVVVLSFELLNAPATVCGPGSGGSGS | |
| | | original RBD | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP625 | (R)-mosaic-SpFN | RBD from KC881007. | NITNLCPFGEVFNATTFPSVYAWERKRISNCVADYSVLYNSTSFSTFKCYGVSATKLNDLCFSNVYADS | 262 |
| | | Bat.WIV1_mosaicSpFN with | FVVKGDDVRQIAPGQTGVIADYNYKLPDDFTGCVLAWNTRNIDATQTGNYNYKYRSLRHGKLRPFER | |
| | | SARS-1 replacing the | DISNVPFSPDGKPCTPPAFNCYWPLNDYGFYITNGIGYQPYRVVVLSFELLNAPATVCGPGSGGSGSS | |
| | | original RBD | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP626 | (R)-mosaic-SpFN | RBD AVP78042_from | NITNVCPFHKVFNATRFPSVYAWERTKISDCIADYTVFYNSTSFSTFKCYGVSPSKLIDLCFTSVYADTF | 263 |
| | | BatSARSlikecoronavirus_ | LIRFSEVRQVAPGQTGVIADYNYKLPDDFTGCVIAWNTAKQDTGHYFYRSHRSTKLKPFERDLSSDEN | |
| | | mosaicSpFN with SARS-1 | GVRTLSTYDFNPNVPLEYQATRVVVLSFELLNAPATVCGPGSGGSGSSQCVNLTTRTQLPPAYTNSFT | |
| | | replacing the original RBD | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDWVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP627 | (R)-mosaic-SpFN | RBD from MZ937001_ | NITNLCPFGEVFNATTFASVYAWNRKRISNCVADYSVLYNSTSFSTFKCYGVSPTKLNDLCFTNVYADS | 264 |
| | | BANAL20103_Laos_2020_ | FVVRGDEVRQIAPGQTGKIADYNYKLPDDFTGCVIAWNSNNLDSKVGGNYNYLYRLFRKSNLKPFERD | |
| | | mosaicSpFN with SARS-1 | ISTEIYQAGSTPCNGVEGFNCYFPLQSYGFHPTNGVGYQPYRVVVLSFELLNAPATVCGPGSGGSGS | |
| | | replacing the original RBD | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP628 | (R)-mosaic-SpFN | RBD from MN996532.2013. | NITNLCPFGEVFNATTFASVYAWNRKRISNCVADYSVLYNSTSFSTFKCYGVSPTKLNDLCFTNVYADS | 265 |
| | | Bat.RaTG13_mosaicSpFN | FVITGDEVRQIAPGQTGKIADYNYKLPDDFTGCVIAWNSKHIDAKEGGNFNYLYRLFRKANLKPFERDI | |
| | | with SARS-1 replacing the | STEIYQAGSKPCNGQTGLNCYYPLYRYGFYPTDGVGHQPYRVVVLSFELLNAPATVCGPGSGGSGSS | |
| | | original RBD | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNWVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP629 | (R)-mosaic-SpFN | RBD from MZ937004_ | NITNFCPFDKVFNATRFPNVYAWQRTKISDCIADYTVLYNSTSFSTFKCYGVSPSKLIDLCFTSVYADTF | 266 |
| | | BANAL20247_Laos_2020_ | LIRFSEVRQIAPGETGVIADYNYKLPDDFTGCVLAWNTAQQDIGSYFYRSHRAVKLKPFERDLSSDEN | |
| | | mosaicSpFN with SARS-1 | GVRTLSTYDFNPNVPLDYQATRVVVLSFELLNAPATVCGPGSGGSGSSQCVNLTTRTQLPPAYTNSF | |
| | | replacing the original RBD | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRENGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP630 | (R)-mosaic-SpFN | BANAL-20-247 RBD-N498W_ | NITNFCPFDKVFNATRFPNVYAWQRTKISDCIADYTVLYNSTSFSTFKCYGVSPSKLIDLCFTSVYADTF | 267 |
| | | mosaicSpFN with SARS-1 | LIRFSEVRQIAPGETGVIADYNYKLPDDFTGCVLAWNTAQQDIGSYFYRSHRAVKLKPFERDLSSDEN | |
| | | replacing the original RBD | GVRTLSTYDFWPNVPLDYQATRVVVLSFELLNAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP631 | (R)-mosaic-SpFN | BANAL-20-247 RBD-S493Y- | NITNFCPFDKVFNATRFPNVYAWQRTKISDCIADYTVLYNSTSFSTFKCYGVSPSKLIDLCFTSVYADTF | 268 |
| | | N498W_mosaicSpFN with | LIRFSEVRQIAPGETGVIADYNYKLPDDFTGCVLAWNTAQQDIGSYFYRSHRAVKLKPFERDLSSDEN | |
| | | SARS-1 replacing the | GVRTLYTYDFWPNVPLDYQATRVVVLSFELLNAPATVCGPGSGGSGSSQCVNLTTRTQLPPAYTNSF | |
| | | original RBD | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP632 | (R)-mosaic-SpFN | RBD from ATO98145_ | NITNLCPFDAVFNATRFPSVYAWERVKISNCVADYTAFYNSTSFSTFKCYGVSPSKLIDLCFTSVYADT | 269 |
| | | BatSARSlikecoronavirus_ | FLIRFSEVRQVAPGETGVIADYNYKLPDDFTGCVIAWNTAKQDVGSYFYRSHRSSKLKPFERDLSSDE | |
| | | mosaicSpFN with SARS-1 | NGVRTLSTYDFNPNVPLDYQATRVVVLSFELLNAPATVCGPGSGGSGSSQCVNLTTRTQLPPAYTNS | |
| | | replacing the original RBD | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATKFPSVYAWERKKISNCVADYSVLYNSTFFSTF | |
| | | | KCYGVSATKLNDLCFSNVYADSFVVKGDDVRQIAPGQTGVIADYNYKLPDDFMGCVLAWNTRNIDAT | |
| | | | STGNYNYKYRYLRHGKLRPFERDISNVPFSPDGKPCTPPALNCYWPLNDYGFYTTTGIGYQPYRVVVL | |
| | | | SFELLNAPATVCGPKKSTNLVKNKCVNFNFNGLTGTGVLTESNKKFLPFQQFGRDIADTTDAVRDPQT | |
| | | | LEILDITPCSFGGVSVITPGTNTSNQVAVLYQDVNCTEVPVAIHADQLTPTWRVYSTGSNVFQTRAGCL | |
| | | | IGAEHVNNSYECDIPIGAGICASYQTQTNSPGSASSVASQSIIAYTMSLGAENSVAYSNNSIAIPTNFTIS | |
| | | | VTTEILPVSMTKTSVDCTMYICGDSTECSNLLLQYGSFCTQLNRALTGIAVEQDKNTQEVFAQVKQIYK | |
| | | | TPPIKDFGGFNFSQILPDPSKPSKRSFIEDLLFNKVTLADAGFIKQYGDCLGDIAARDLICAQKFNGLTVL | |
| | | | PPLLTDEMIAQYTSALLAGTITSGWTFGAGAALQIPFAMQMAYRFNGIGVTQNVLYENQKLIANQFNSA | |
| | | | IGKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP633 | (R)-mosaic-SpFN | RBD from JX993987.2011. | NITNRCPFDKVFNATRFPSVYAWERTKISDCVADYTVLYNSTSFSTFKCYGVSPSKLIDLCFTSVYADT | 270 |
| | | BatRp.Shaanxi2011_ | FLIRSSEVRQVAPGETGVIADYNYKLPDDFTGCVIAWNTANQDQGQYYYRSSRKEKLKPFERDLSSDE | |
| | | mosaicSpFN with SARS-1 | NGVYTLSTYDFYPSVPLDYQATRVVVLSFELLNAPATVCGPGSGGSGSSQCVNLTTRTQLPPAYTNSF | |
| | | replacing the original RBD | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP634 | (R)-mosaic-SpFN | RBD from KJ473812. | NITNLCPFDKVFNATRFPSVYAWERTKISDCVADYTVFYNSTSFSTFNCYGVSPSKLIDLCFTSVYADT | 271 |
| | | BtRfBetaCoV.HeB2013_ | FLIRFSEVRQVAPGQTGVIADYNYKLPDDFTGCVIAWNTAKQDVGSYFYRSHRSSKLKPFERDLSSEE | |
| | | mosaicSpFN with SARS-1 | NGVRTLSTYDFNQYVPLEYQATRVVVLSFELLNAPATVCGPGSGGSGSSQCVNLTTRTQLPPAYTNS | |
| | | replacing the original RBD | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATKFPSVYAWERKKISNCVADYSVLYNSTFFSTF | |
| | | | KCYGVSATKLNDLCFSNVYADSFVVKGDDVRQIAPGQTGVIADYNYKLPDDFMGCVLAWNTRNIDAT | |
| | | | STGNYNYKYRYLRHGKLRPFERDISNVPFSPDGKPCTPPALNCYWPLNDYGFYTTTGIGYQPYRVVVL | |
| | | | SFELLNAPATVCGPKKSTNLVKNKCVNFNFNGLTGTGVLTESNKKFLPFQQFGRDIADTTDAVRDPQT | |
| | | | LEILDITPCSFGGVSVITPGTNTSNQVAVLYQDVNCTEVPVAIHADQLTPTWRVYSTGSNVFQTRAGCL | |
| | | | IGAEHVNNSYECDIPIGAGICASYQTQTNSPGSASSVASQSIIAYTMSLGAENSVAYSNNSIAIPTNFTIS | |
| | | | VTTEILPVSMTKTSVDCTMYICGDSTECSNLLLQYGSFCTQLNRALTGIAVEQDKNTQEVFAQVKQIYK | |
| | | | TPPIKDFGGFNFSQILPDPSKPSKRSFIEDLLFNKVTLADAGFIKQYGDCLGDIAARDLICAQKFNGLTVL | |
| | | | PPLLTDEMIAQYTSALLAGTITSGWTFGAGAALQIPFAMQMAYRFNGIGVTQNVLYENQKLIANQFNSA | |
| | | | IGKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP635 | (R)-mosaic-SpFN | RBD from DQ071615.2003. | NITNRCPFDKVFNATRFPNVYAWERTKISDCVADYTVLYNSTSFSTFKCYGVSPSKLIDLCFTSVYADT | 272 |
| | | Bat.Rp3_mosaicSpFN with | FLIRSSEVRQVAPGETGVIADYNYKLPDDFTGCVIAWNTAKQDQGQYYYRSHRKTKLKPFERDLSSDE | |
| | | SARS-1 replacing the | NGVRTLSTYDFYPSVPVAYQATRVVVLSFELLNAPATVCGPGSGGSGSSQCVNLTTRTQLPPAYTNS | |
| | | original RBD | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATKFPSVYAWERKKISNCVADYSVLYNSTFFSTF | |
| | | | KCYGVSATKLNDLCFSNVYADSFVVKGDDVRQIAPGQTGVIADYNYKLPDDFMGCVLAWNTRNIDAT | |
| | | | STGNYNYKYRYLRHGKLRPFERDISNVPFSPDGKPCTPPALNCYWPLNDYGFYTTTGIGYQPYRVWVL | |
| | | | SFELLNAPATVCGPKKSTNLVKNKCVNFNFNGLTGTGVLTESNKKFLPFQQFGRDIADTTDAVRDPQT | |
| | | | LEILDITPCSFGGVSVITPGTNTSNQVAVLYQDVNCTEVPVAIHADQLTPTWRVYSTGSNVFQTRAGCL | |
| | | | IGAEHVNNSYECDIPIGAGICASYQTQTNSPGSASSVASQSIIAYTMSLGAENSVAYSNNSIAIPTNFTIS | |
| | | | VTTEILPVSMTKTSVDCTMYICGDSTECSNLLLQYGSFCTQLNRALTGIAVEQDKNTQEVFAQVKQIYK | |
| | | | TPPIKDFGGFNFSQILPDPSKPSKRSFIEDLLFNKVTLADAGFIKQYGDCLGDIAARDLICAQKFNGLTVL | |
| | | | PPLLTDEMIAQYTSALLAGTITSGWTFGAGAALQIPFAMQMAYRFNGIGVTQNVLYENQKLIANQFNSA | |
| | | | IGKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP636 | (R)-mosaic-SpFN | RBD from ACU31032_ | NITNRCPFDKVFNASRFPNVYAWERTKISDCVADYTVLYNSTSFSTFKCYGVSPSKLIDLCFTSVYADT | 273 |
| | | Rs_672_2006_mosaicSpFN | FLIRSSEVRQVAPGETGVIADYNYKLPDDFTGCVIAWNTAKQDQGQYYYRSSRKTKLKPFERDLTSDE | |
| | | with SARS-1 replacing the | NGVRTLSTYDFYPNVPIEYQATRVVVLSFELLNAPATVCGPGSGGSGSSQCVNLTTRTQLPPAYTNSF | |
| | | original RBD | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP637 | (R)-mosaic-SpFN | RBD from MZ081380_bat_ | NITNLCPFSQVFNATRFPSVYAWTRERISNCIADYSVLYNSTSFSTFRCYGVSPTKLNDLCFSNVYADS | 274 |
| | | Yunnan_RsYN04_2020_ | MVVRGDEVRQIAPSQTGVIADYNYKLPDDFTGCVIAWNSKAKDENGQYFYRLFRKSKLLPFQRDVSN | |
| | | mosaicSpFN with SARS-1 | VTYGSGKNDGCNPSEADCYWPLLKYGFTSSVSQDYQPYRVVVLSFELLNAPATVCGPGSGGSGSSQ | |
| | | replacing the original RBD | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP638 | (R)-mosaic-SpFN | RsYN04 RBD-T498W_ | NITNLCPFSQVFNATRFPSVYAWTRERISNCIADYSVLYNSTSFSTFRCYGVSPTKLNDLCFSNVYADS | 275 |
| | | mosaicSpFN with SARS-1 | MVVRGDEVRQIAPSQTGVIADYNYKLPDDFTGCVIAWNSKAKDENGQYFYRLFRKSKLLPFQRDVSN | |
| | | replacing the original RBD | VTYGSGKNDGCNPSEADCYWPLLKYGFWSSVSQDYQPYRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP639 | (R)-mosaic-SpFN | RsYN04 RBD-L493Y-T498W_ | NITNLCPFSQVFNATRFPSVYAWTRERISNCIADYSVLYNSTSFSTFRCYGVSPTKLNDLCFSNVYADS | 276 |
| | | mosaicSpFN with SARS-1 | MVVRGDEVRQIAPSQTGVIADYNYKLPDDFTGCVIAWNSKAKDENGQYFYRLFRKSKLLPFQRDVSN | |
| | | replacing the original RBD | VTYGSGKNDGCNPSEADCYWPLYKYGFWSSVSQDYQPYRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP640 | (R)-mosaic-SpFN | Khosta2-MZ190138 _ | NMTNICPFDQVFNKTQFPSVYAWERVRISDCVSDYTVLYNSSASFSTFKCYGVSPTKLNDLCFSGVYA | 277 |
| | | mosaicSpFN with SARS-1 | DYFWKGDHVHQIAPGQTGVIADYNYKLPSEFVGCILAWNTRTIDSKRGFYYRLFRHGNIRPYERDTSN | |
| | | replacing | VPYNAAGGTCNQPGTHNCYEPLQDYGFTSTSGVGYQPFRVVVLSFELLNAPATVCGPGSGGSGSSQ | |
| | | the original RBD | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP641 | (R)-mosaic-SpFN | Khosta-2 RBD-T498W_ | NMTNICPFDQVFNKTQFPSVYAWERVRISDCVSDYTVLYNSSASFSTFKCYGVSPTKLNDLCFSGVYA | 278 |
| | | mosaicSpFN with SARS-1 | DYFWVKGDHVHQIAPGQTGVIADYNYKLPSEFVGCILAWNTRTIDSKRGFYYRLFRHGNIRPYERDTSN | |
| | | replacing the original RBD | VPYNAAGGTCNQPGTHNCYEPLQDYGFWSTSGVGYQPFRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP642 | (R)-mosaic-SpFN | Khosta-2 RBD-Q493Y-T498W_ | NMTNICPFDQVFNKTQFPSVYAWERVRISDCVSDYTVLYNSSASFSTFKCYGVSPTKLNDLCFSGVYA | 279 |
| | | mosaicSpFN with SARS-1 | DYFWVKGDHVHQIAPGQTGVIADYNYKLPSEFVGCILAWNTRTIDSKRGFYYRLFRHGNIRPYERDTSN | |
| | | replacing the original RBD | VPYNAAGGTCNQPGTHNCYEPLYDYGFWSTSGVGYQPFRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP643 | (R)-mosaic-SpFN | KY352407_BtKY72_bat_Kenya_ | NITNLCPFGQVFNASNFPSVYAWERLRISDCVADYAVLYNSSSSFSTFKCYGVSPTKLNDLCFSSVYA | 280 |
| | | mosaicSpFN with SARS- | DYFWVKGDDVRQIAPAQTGVIADYNYKLPDDFTGCVLAWNTNSVDSKSGNNFYYRLFRHGKIKPYER | |
| | | 1 replacing the original | DISNVLYNSAGGTCSSISQLGCYEPLKSYGFTPTVGVGYQPYRVVVLSFELLNAPATVCGPGSGGSGS | |
| | | RBD | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFWVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP644 | (R)-mosaic-SpFN | BtKY72 RBD-T498W_ | NITNLCPFGQVFNASNFPSVYAWERLRISDCVADYAVLYNSSSSFSTFKCYGVSPTKLNDLCFSSVYA | 281 |
| | | mosaicSpFN with SARS-1 | DYFWVKGDDVRQIAPAQTGVIADYNYKLPDDFTGCVLAWNTNSVDSKSGNNFYYRLFRHGKIKPYER | |
| | | replacing the original RBD | DISNVLYNSAGGTCSSISQLGCYEPLKSYGFWPTVGVGYQPYRVVVLSFELLNAPATVCGPGSGGSG | |
| | | | SSQCVNLTTRTQLPPAYTNSFTRGVYYPDKVFRSSVLHSTQDLFLPFFSNVTWFHAIHVSGTNGTKRF | |
| | | | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATKFPSVYAWERK | |
| | | | KISNCVADYSVLYNSTFFSTFKCYGVSATKLNDLCFSNVYADSFVVKGDDVRQIAPGQTGVIADYNYKL | |
| | | | PDDFMGCVLAWNTRNIDATSTGNYNYKYRYLRHGKLRPFERDISNVPFSPDGKPCTPPALNCYWPLN | |
| | | | DYGFYTTTGIGYQPYRVVVLSFELLNAPATVCGPKKSTNLVKNKCVNFNFNGLTGTGVLTESNKKFLP | |
| | | | FQQFGRDIADTTDAVRDPQTLEILDITPCSFGGVSVITPGTNTSNQVAVLYQDVNCTEVPVAIHADQLT | |
| | | | PTWRVYSTGSNVFQTRAGCLIGAEHVNNSYECDIPIGAGICASYQTQTNSPGSASSVASQSIIAYTMSL | |
| | | | GAENSVAYSNNSIAIPTNFTISVTTEILPVSMTKTSVDCTMYICGDSTECSNLLLQYGSFCTQLNRALTGI | |
| | | | AVEQDKNTQEVFAQVKQIYKTPPIKDFGGFNFSQILPDPSKPSKRSFIEDLLFNKVTLADAGFIKQYGDC | |
| | | | LGDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP645 | (R)-mosaic-SpFN | BtKY72 RBD-K493Y-T498W_ | NITNLCPFGQVFNASNFPSVYAWERLRISDCVADYAVLYNSSSSFSTFKCYGVSPTKLNDLCFSSVYA | 282 |
| | | mosaicSpFN with SARS-1 | DYFWVKGDDVRQIAPAQTGVIADYNYKLPDDFTGCVLAWNTNSVDSKSGNNFYYRLFRHGKIKPYER | |
| | | replacing the original RBD | DISNVLYNSAGGTCSSISQLGCYEPLYSYGFWPTVGVGYQPYRVVVLSFELLNAPATVCGPGSGGSG | |
| | | | SSQCVNLTTRTQLPPAYTNSFTRGVYYPDKVFRSSVLHSTQDLFLPFFSNVTWFHAIHVSGTNGTKRF | |
| | | | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATKFPSVYAWERK | |
| | | | KISNCVADYSVLYNSTFFSTFKCYGVSATKLNDLCFSNVYADSFVVKGDDVRQIAPGQTGVIADYNYKL | |
| | | | PDDFMGCVLAWNTRNIDATSTGNYNYKYRYLRHGKLRPFERDISNVPFSPDGKPCTPPALNCYWPLN | |
| | | | DYGFYTTTGIGYQPYRVVVLSFELLNAPATVCGPKKSTNLVKNKCVNFNFNGLTGTGVLTESNKKFLP | |
| | | | FQQFGRDIADTTDAVRDPQTLEILDITPCSFGGVSVITPGTNTSNQVAVLYQDVNCTEVPVAIHADQLT | |
| | | | PTWRVYSTGSNVFQTRAGCLIGAEHVNNSYECDIPIGAGICASYQTQTNSPGSASSVASQSIIAYTMSL | |
| | | | GAENSVAYSNNSIAIPTNFTISVTTEILPVSMTKTSVDCTMYICGDSTECSNLLLQYGSFCTQLNRALTGI | |
| | | | AVEQDKNTQEVFAQVKQIYKTPPIKDFGGFNFSQILPDPSKPSKRSFIEDLLFNKVTLADAGFIKQYGDC | |
| | | | LGDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP646 | (R)-mosaic-SpFN | RaTG15_Ra7909 (RBD)_ | NITNLCPFSQVFNATRFPSVYAWTRERISNCIADYSVLYNSTSFSTFRCYGVSPTKLNDLCFSNVYADS | 283 |
| | | mosaicSpFN with SARS-1 | MVVRGDEVRQIAPSQTGVIADYNYKLPDDFTGCVIAWNSKAKDENGQYFYRLFRKSKLLPFQRDVSN | |
| | | replacing the original RBD | VTYGSGKNDGCNPSEADCYWPLLKYGFTGSVSQDYQPYRVVVLSFELLNAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP647 | (R)-mosaic-SpFN | HKU3-1_DQ022305.2005 | NITNRCPFDKVFNATRFPNVYAWERTKISDCVADYTVLYNSTSFSTFKCYGVSPSKLIDLCFTSVYADT | 284 |
| | | (RBD)_mosaicSpFN with SARS- | FLIRSSEVRQVAPGETGVIADYNYKLPDDFTGCVIAWNTAKHDTGNYYYRSHRKTKLKPFERDLSSDD | |
| | | 1 replacing the original | GNGVYTLSTYDFNPNVPVAYQATRVVVLSFELLNAPATVCGPGSGGSGSSQCVNLTTRTQLPPAYTN | |
| | | RBD | SFTRGVYYPDKVFRSSVLHSTQDLFLPFFSNVTWFHAIHVSGTNGTKRFDNPVLPFNDGVYFASTEKS | |
| | | | NIIRGWIFGTTLDSKTQSLLIVNNATNVVIKVCEFQFCNDPFLGVYYHKNNKSWMESEFRVYSSANNCT | |
| | | | FEYVSQPFLMDLEGKQGNFKNLREFVFKNIDGYFKIYSKHTPINLVRDLPQGFSALEPLVDLPIGINITRF | |
| | | | QTLLALHRSYLTPGDSSSGWTAGAAAYYVGYLQPRTFLLKYNENGTITDAVDCALDPLSETKCTLKSF | |
| | | | TVEKGIYQTSNFRVQPTESIVRFPNITNLCPFGEVFNATKFPSVYAWERKKISNCVADYSVLYNSTFFS | |
| | | | TFKCYGVSATKLNDLCFSNVYADSFVVKGDDVRQIAPGQTGVIADYNYKLPDDFMGCVLAWNTRNIDA | |
| | | | TSTGNYNYKYRYLRHGKLRPFERDISNVPFSPDGKPCTPPALNCYWPLNDYGFYTTTGIGYQPYRWV | |
| | | | VLSFELLNAPATVCGPKKSTNLVKNKCVNFNFNGLTGTGVLTESNKKFLPFQQFGRDIADTTDAVRDP | |
| | | | QTLEILDITPCSFGGVSVITPGTNTSNQVAVLYQDVNCTEVPVAIHADQLTPTWRVYSTGSNVFQTRAG | |
| | | | CLIGAEHVNNSYECDIPIGAGICASYQTQTNSPGSASSVASQSIIAYTMSLGAENSVAYSNNSIAIPTNFT | |
| | | | ISVTTEILPVSMTKTSVDCTMYICGDSTECSNLLLQYGSFCTQLNRALTGIAVEQDKNTQEVFAQVKQIY | |
| | | | KTPPIKDFGGFNFSQILPDPSKPSKRSFIEDLLFNKVTLADAGFIKQYGDCLGDIAARDLICAQKFNGLTV | |
| | | | LPPLLTDEMIAQYTSALLAGTITSGWTFGAGAALQIPFAMQMAYRFNGIGVTQNVLYENQKLIANQFNS | |
| | | | AIGKIQDSLSSTASALGKLQDVVNQNAQALNTLVKQLSSNFGAISSVLNDILSRLDPPEAEVQIDRLITGR | |
| | | | LQSLQTYVTQQLIRAAEIRASANLAATKMSECVLGQSKRVDFCGKGYHLMSFPQSAPHGVVFLHVTYV | |
| | | | PAQEKNFTTAPAICHDGKAHFPREGVFVSNGTHWFVTQRNFYEPQIITTDNTFVSGNCDVVIGIVNNTV | |
| | | | YDPLQSELDSIKEELDKIHKNGSGGSGDIIKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP648 | (R)-mosaic-SpFN | RhGB01-MW719567_mosaicSpFN | NVTQLCPFSDVFNRTMFPSVYAWERMSIRDCVADYAVLYNSSISFSTFECYGVSPTKLNDLCFSSVYA | 285 |
| | | with SARS-1 replacing | DYFWVRGDDVKQIAPGKTGVIADYNYKLPDDFVGCVLAWNSRHVDNKQGFYYRLFRHGKIKPYQRDM | |
| | | the original RBD | SRELYSPSGTPCTVVGFNCYDPLAHYAFASPGIGIGYQPFRVVVLSFQLLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP649 | (R)-mosaic-SpFN | bioinformatically derived | NITNLCPFDEVFNATRFPSVYAWERKKISNCVADYSVLYNSTSFSTFKCYGVSPSKLNDLCFTSVYADS | 286 |
| | | Cluster1_RBD (SARS- | FVIRGDEVRQIAPGQTGVIADYNYKLPDDFTGCVIAWNTANQDSGEGGNPNYYYRSSRKSKLKPFER | |
| | | related)_ | DLSSDEYNVGSQPCNRMGPNCYRTLSTYDFYPTVGVGYQPYRVVVLSFELLNAPATVCGPGSGGSG | |
| | | mosaicSpFN with SARS-1 | SSQCVNLTTRTQLPPAYTNSFTRGVYYPDKVFRSSVLHSTQDLFLPFFSNVTWFHAIHVSGTNGTKRF | |
| | | replacing the original RBD | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATKFPSVYAWERK | |
| | | | KISNCVADYSVLYNSTFFSTFKCYGVSATKLNDLCFSNVYADSFVVKGDDVRQIAPGQTGVIADYNYKL | |
| | | | PDDFMGCVLAWNTRNIDATSTGNYNYKYRYLRHGKLRPFERDISNVPFSPDGKPCTPPALNCYWPLN | |
| | | | DYGFYTTTGIGYQPYRVVVLSFELLNAPATVCGPKKSTNLVKNKCVNFNFNGLTGTGVLTESNKKFLP | |
| | | | FQQFGRDIADTTDAVRDPQTLEILDITPCSFGGVSVITPGTNTSNQVAVLYQDVNCTEVPVAIHADQLT | |
| | | | PTWRVYSTGSNVFQTRAGCLIGAEHVNNSYECDIPIGAGICASYQTQTNSPGSASSVASQSIIAYTMSL | |
| | | | GAENSVAYSNNSIAIPTNFTISVTTEILPVSMTKTSVDCTMYICGDSTECSNLLLQYGSFCTQLNRALTGI | |
| | | | AVEQDKNTQEVFAQVKQIYKTPPIKDFGGFNFSQILPDPSKPSKRSFIEDLLFNKVTLADAGFIKQYGDC | |
| | | | LGDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP650 | (R)-mosaic-SpFN | bioinformatically derived | NLPDCNIEEWLTAKSVPSPLNWERKTFSNCNFNLSSLLRFVQADSFSCNNIDASKVYGMCFGSISVDK | 287 |
| | | Cluster2_RBD (OC43- | FAIPNSRQVDLQLGNSGFLQSFNYKIDTTATSCQLYYSLPANNVTINNYNPSSWNRRYGFNDDTVFKP | |
| | | related)_ | RPAGVLTNHDVVYSQQCFKVPNTFCPCKNNGSCVSSGKGTTNGSGTCPAGTNYRTCENTTWCACS | |
| | | mosaicSpFN with SARS-1 | CTPDPITASGPRKCPQKKSLVGVGEHCSGLGVDSDYCGNVSCLCSTDAFLGWSADSCLVNDRCNIFA | |
| | | replacing the original RBD | NIILNGINSGTTCSTGSGGSGSSQCVNLTTRTQLPPAYTNSFTRGVYYPDKVFRSSVLHSTQDLFLPFF | |
| | | | SNVTWFHAIHVSGTNGTKRFDNPVLPFNDGVYFASTEKSNIIRGWIFGTTLDSKTQSLLIVNNATNVVIK | |
| | | | VCEFQFCNDPFLGVYYHKNNKSWMESEFRVYSSANNCTFEYVSQPFLMDLEGKQGNFKNLREFVFK | |
| | | | NIDGYFKIYSKHTPINLVRDLPQGFSALEPLVDLPIGINITRFQTLLALHRSYLTPGDSSSGWTAGAAAYY | |
| | | | VGYLQPRTFLLKYNENGTITDAVDCALDPLSETKCTLKSFTVEKGIYQTSNFRVQPTESIVRFPNITNLC | |
| | | | PFGEVFNATKFPSVYAWERKKISNCVADYSVLYNSTFFSTFKCYGVSATKLNDLCFSNVYADSFVVKG | |
| | | | DDVRQIAPGQTGVIADYNYKLPDDFMGCVLAWNTRNIDATSTGNYNYKYRYLRHGKLRPFERDISNVP | |
| | | | FSPDGKPCTPPALNCYWPLNDYGFYTTTGIGYQPYRVVVLSFELLNAPATVCGPKKSTNLVKNKCVNF | |
| | | | NFNGLTGTGVLTESNKKFLPFQQFGRDIADTTDAVRDPQTLEILDITPCSFGGVSVITPGTNTSNQVAV | |
| | | | LYQDVNCTEVPVAIHADQLTPTWRVYSTGSNVFQTRAGCLIGAEHVNNSYECDIPIGAGICASYQTQT | |
| | | | NSPGSASSVASQSIIAYTMSLGAENSVAYSNNSIAIPTNFTISVTTEILPVSMTKTSVDCTMYICGDSTEC | |
| | | | SNLLLQYGSFCTQLNRALTGIAVEQDKNTQEVFAQVKQIYKTPPIKDFGGFNFSQILPDPSKPSKRSFIE | |
| | | | DLLFNKVTLADAGFIKQYGDCLGDIAARDLICAQKFNGLTVLPPLLTDEMIAQYTSALLAGTITSGWTFG | |
| | | | AGAALQIPFAMQMAYRFNGIGVTQNVLYENQKLIANQFNSAIGKIQDSLSSTASALGKLQDVVNQNAQ | |
| | | | ALNTLVKQLSSNFGAISSVLNDILSRLDPPEAEVQIDRLITGRLQSLQTYVTQQLIRAAEIRASANLAATK | |
| | | | MSECVLGQSKRVDFCGKGYHLMSFPQSAPHGVVFLHVTYVPAQEKNFTTAPAICHDGKAHFPREGVF | |
| | | | VSNGTHWFVTQRNFYEPQIITTDNTFVSGNCDVVIGIVNNTVYDPLQSELDSIKEELDKIHKNGSGGSG | |
| | | | DIIKLLNEQVNKEMQSSNLYMSMSSWCYTHSLDGAGLFLFDHAAEEYEHAKKLIIFLNENNVPVQLTSIS | |
| | | | APEHKFEGLTQIFQKAYEHEQHISESINNIVDHAIKSKDHATFNFLQWYVAEQHEEEVLFKDILDKIELIG | |
| | | | NENHGLYLADQYVKGIAKSRKSGS | |
| pCoVP651 | (R)-mosaic-SpFN | bioinformatically derived | QGVECDFSKLFKAAAPQIYNFSRLVFTNCNYNLTKLLSLFQVDEFSCHQISPSALASGCYSSLTVDYFA | 288 |
| | | Cluster3_RBD (MERS- | YPLSMKSYLQIGSAGEISQFNYKQDFSNPTCRILASVPANVSITKPDKYIYISQCYSLSAYSDVDVPHYV | |
| | | related)_ | NPGQYTSIPCLYLTSSGFDEDGQYFRYNLTQFEGQNYLVATGVTVSMTDNLQMVFVITVQYGTDTNS | |
| | | mosaicSpFN with SARS-1 | VCPGSGGSGSSQCVNLTTRTQLPPAYTNSFTRGVYYPDKVFRSSVLHSTQDLFLPFFSNVTWFHAIH | |
| | | replacing the original RBD | VSGTNGTKRFDNPVLPFNDGVYFASTEKSNIIRGWIFGTTLDSKTQSLLIVNNATNVVIKVCEFQFQND | |
| | | | PFLGVYYHKNNKSWMESEFRVYSSANNCTFEYVSQPFLMDLEGKQGNFKNLREFVFKNIDGYFKIYS | |
| | | | KHTPINLVRDLPQGFSALEPLVDLPIGINITRFQTLLALHRSYLTPGDSSSGWTAGAAAYYVGYLQPRTF | |
| | | | LLKYNENGTITDAVDCALDPLSETKCTLKSFTVEKGIYQTSNFRVQPTESIVRFPNITNLCPFGEVFNAT | |
| | | | KFPSVYAWERKKISNCVADYSVLYNSTFFSTFKCYGVSATKLNDLCFSNVYADSFVVKGDDVRQIAPG | |
| | | | QTGVIADYNYKLPDDFMGCVLAWNTRNIDATSTGNYNYKYRYLRHGKLRPFERDISNVPFSPDGKPCT | |
| | | | PPALNCYWPLNDYGFYTTTGIGYQPYRVVVLSFELLNAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDVVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoVP652 | (R)-mosaic-SpFN | MER1 (ErinaceusCoV/2012- | ELTECDLDVLFKNDAPIIANYSRRVFTNCNYNLTKLLSLVQVDEFVCHKTTPEALATGCYSSLTVDWFA | 289 |
| | | 174/GER/2012)_mosaicSpFN | LPFSMKSTLAIGSAEAISMFNYNQDYSNPTCRIHAAVTANVSTALNFTANANYAYISRCQGVDGKPILLQ | |
| | | with SARS-1 replacing the | PGQMTNIACRSGVLARPSDADYFGYSFQGRNYYLGRKSYKPKTDEGDVQMVYVITPKYDKGPDTVC | |
| | | original RBD | PLKGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATKF | |
| | | | PSVYAWERKKISNCVADYSVLYNSTFFSTFKCYGVSATKLNDLCFSNVYADSFVVKGDDVRQIAPGQT | |
| | | | GVIADYNYKLPDDFMGCVLAWNTRNIDATSTGNYNYKYRYLRHGKLRPFERDISNVPFSPDGKPCTPP | |
| | | | ALNCYWPLNDYGFYTTTGIGYQPYRVVVLSFELLNAPATVCGPKKSTNLVKNKCVNFNFNGLTGTGVL | |
| | | | TESNKKFLPFQQFGRDIADTTDAVRDPQTLEILDITPCSFGGVSVITPGTNTSNQVAVLYQDVNCTEVP | |
| | | | VAIHADQLTPTWRVYSTGSNVFQTRAGCLIGAEHVNNSYECDIPIGAGICASYQTQTNSPGSASSVAS | |
| | | | QSIIAYTMSLGAENSVAYSNNSIAIPTNFTISVTTEILPVSMTKTSVDCTMYICGDSTECSNLLLQYGSFC | |
| | | | TQLNRALTGIAVEQDKNTQEVFAQVKQIYKTPPIKDFGGFNFSQILPDPSKPSKRSFIEDLLFNKVTLAD | |
| | | | AGFIKQYGDCLGDIAARDLICAQKFNGLTVLPPLLTDEMIAQYTSALLAGTITSGWTFGAGAALQIPFAM | |
| | | | QMAYRFNGIGVTQNVLYENQKLIANQFNSAIGKIQDSLSSTASALGKLQDVVNQNAQALNTLVKQLSS | |
| | | | NFGAISSVLNDILSRLDPPEAEVQIDRLITGRLQSLQTYVTQQLIRAAEIRASANLAATKMSECVLGQSK | |
| | | | RVDFCGKGYHLMSFPQSAPHGVVFLHVTYVPAQEKNFTTAPAICHDGKAHFPREGVFVSNGTHWFV | |
| | | | TQRNFYEPQIITTDNTFVSGNCDVVIGIVNNTVYDPLQSELDSIKEELDKIHKNGSGGSGDIIKLLNEQVN | |
| | | | KEMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoVP653 | (R)-mosaic-SpFN | MER2 (Neoromicia/5038)_ | VYPDCNFTELFRERAPTIMQYKREVFTRCNYNLSLLLSLVQVDEFVCDKITPEALATGCYSSLTVDWFA | 290 |
| | | mosaicSpFN with SARS-1 | FPYAWKSYLAIGSADRIVRFNYNQDYSNPSCRIHSKVNSSIGISYAGAYSYITNCNYGATNKDDVVKPG | |
| | | replacing the original RBD | GRASQQCITGALNSPTTGQLWAYNFGGVPYRVSRLTYTDHLSDPLDMVYVITVKYEPGAETVCPKQG | |
| | | | SGGSGSSQCVNLTTRTQLPPAYTNSFTRGVYYPDKVFRSSVLHSTQDLFLPFFSNVTWFHAIHVSGT | |
| | | | NGTKRFDNPVLPFNDGVYFASTEKSNIIRGWIFGTTLDSKTQSLLIVNNATNVVIKVCEFQFCNDPFLGV | |
| | | | YYHKNNKSWMESEFRVYSSANNCTFEYVSQPFLMDLEGKQGNFKNLREFVFKNIDGYFKIYSKHTPIN | |
| | | | LVRDLPQGFSALEPLVDLPIGINITRFQTLLALHRSYLTPGDSSSGWTAGAAAYYVGYLQPRTFLLKYN | |
| | | | ENGTITDAVDCALDPLSETKCTLKSFTVEKGIYQTSNFRVQPTESIVRFPNITNLCPFGEVFNATKFPSV | |
| | | | YAWERKKISNCVADYSVLYNSTFFSTFKCYGVSATKLNDLCFSNVYADSFVVKGDDVRQIAPGQTGVI | |
| | | | ADYNYKLPDDFMGCVLAWNTRNIDATSTGNYNYKYRYLRHGKLRPFERDISNVPFSPDGKPCTPPAL | |
| | | | NCYWPLNDYGFYTTTGIGYQPYRVVVLSFELLNAPATVCGPKKSTNLVKNKCVNFNFNGLTGTGVLTE | |
| | | | SNKKFLPFQQFGRDIADTTDAVRDPQTLEILDITPCSFGGVSVITPGTNTSNQVAVLYQDVNCTEVPVAI | |
| | | | HADQLTPTWRVYSTGSNVFQTRAGCLIGAEHVNNSYECDIPIGAGICASYQTQTNSPGSASSVASQSII | |
| | | | AYTMSLGAENSVAYSNNSIAIPTNFTISVTTEILPVSMTKTSVDCTMYICGDSTECSNLLLQYGSFCTQL | |
| | | | NRALTGIAVEQDKNTQEVFAQVKQIYKTPPIKDFGGFNFSQILPDPSKPSKRSFIEDLLFNKVTLADAGFI | |
| | | | KQYGDCLGDIAARDLICAQKFNGLTVLPPLLTDEMIAQYTSALLAGTITSGWTFGAGAALQIPFAMQMA | |
| | | | YRFNGIGVTQNVLYENQKLIANQFNSAIGKIQDSLSSTASALGKLQDVVNQNAQALNTLVKQLSSNFGA | |
| | | | ISSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDVVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP654 | (R)-mosaic-SpFN | MER3 (HKU4 SM3A)_ | QATECDFSPMLTGVAPQVYNFKRLVFSNCNYNLTKLLSLFAVDEFSCNGISPDAIARGCYSTLTVDYFA | 291 |
| | | mosaicSpFN with SARS-1 | YPLSMKSYIRPGSAGNIPLYNYKQSFANPTCRVMASVPANVTITKPDAYGYISKCSRLTGANQDVETPL | |
| | | replacing the original RBD | YINPGEYSICRDFSPGGFSEDGQVFKRTLTQFEGGGLLIGVGTRVPMTDNLQMSFIIFVQYGTGTDSV | |
| | | | CPMLGSGGSGSSQCVNLTTRTQLPPAYTNSFTRGVYYPDKVFRSSVLHSTQDLFLPFFSNVTWFHAI | |
| | | | HVSGTNGTKRFDNPVLPFNDGVYFASTEKSNIIRGWIFGTTLDSKTQSLLIVNNATNVVIKVCEFQFCN | |
| | | | DPFLGVYYHKNNKSWMESEFRVYSSANNCTFEYVSQPFLMDLEGKQGNFKNLREFVFKNIDGYFKIY | |
| | | | SKHTPINLVRDLPQGFSALEPLVDLPIGINITRFQTLLALHRSYLTPGDSSSGWTAGAAAYYVGYLQPRT | |
| | | | FLLKYNENGTITDAVDCALDPLSETKCTLKSFTVEKGIYQTSNFRVQPTESIVRFPNITNLCPFGEVFNA | |
| | | | TKFPSVYAWERKKISNCVADYSVLYNSTFFSTFKCYGVSATKLNDLCFSNVYADSFVVKGDDVRQIAP | |
| | | | GQTGVIADYNYKLPDDFMGCVLAWNTRNIDATSTGNYNYKYRYLRHGKLRPFERDISNVPFSPDGKP | |
| | | | CTPPALNCYWPLNDYGFYTTTGIGYQPYRVVVLSFELLNAPATVCGPKKSTNLVKNKCVNFNFNGLTG | |
| | | | TGVLTESNKKFLPFQQFGRDIADTTDAVRDPQTLEILDITPCSFGGVSVITPGTNTSNQVAVLYQDVNC | |
| | | | TEVPVAIHADQLTPTWRVYSTGSNVFQTRAGCLIGAEHVNNSYECDIPIGAGICASYQTQTNSPGSAS | |
| | | | SVASQSIIAYTMSLGAENSVAYSNNSIAIPTNFTISVTTEILPVSMTKTSVDCTMYICGDSTECSNLLLQY | |
| | | | GSFCTQLNRALTGIAVEQDKNTQEVFAQVKQIYKTPPIKDFGGFNFSQILPDPSKPSKRSFIEDLLFNKV | |
| | | | TLADAGFIKQYGDCLGDIAARDLICAQKFNGLTVLPPLLTDEMIAQYTSALLAGTITSGWTFGAGAALQI | |
| | | | PFAMQMAYRFNGIGVTQNVLYENQKLIANQFNSAIGKIQDSLSSTASALGKLQDVVNQNAQALNTLVK | |
| | | | QLSSNFGAISSVLNDILSRLDPPEAEVQIDRLITGRLQSLQTYVTQQLIRAAEIRASANLAATKMSECVLG | |
| | | | QSKRVDFCGKGYHLMSFPQSAPHGVVFLHVTYVPAQEKNFTTAPAICHDGKAHFPREGVFVSNGTH | |
| | | | WFVTQRNFYEPQIITTDNTFVSGNCDWVIGIVNNTVYDPLQSELDSIKEELDKIHKNGSGGSGDIIKLLNE | |
| | | | QVNKEMQSSNLYMSMSSWCYTHSLDGAGLFLFDHAAEEYEHAKKLIIFLNENNVPVQLTSISAPEHKF | |
| | | | EGLTQIFQKAYEHEQHISESINNIVDHAIKSKDHATFNFLQWYVAEQHEEEVLFKDILDKIELIGNENHGL | |
| | | | YLADQYVKGIAKSRKSGS | |
| pCoVP655 | (R)-mosaic-SpFN | MER4 (BatCoV-Ita2 206645- | QGVECDFSKLFKAAPPQIYNFSRLVFTNCNYNLTKLLSLFHVSEFSCHQVSPSALASGCYSSLTVDYFA | 292 |
| | | 63) mosaicSpFN with SARS- | YPLYLASYLQQGSTGEIAQYNYKQDFSNPTCRILASVPANVSIPKPDKYIWLSQCYSFSAYSGDIPHYV | |
| | | 1 replacing the original | QPGQYTPCLYLTSSGFDKSYQTNRDMQNKMAATGVTSSMTDNLQMAFVISVQYGTDTNSVCPMGS | |
| | | RBD | GGSGSSQCVNLTTRTQLPPAYTNSFTRGVYYPDKVFRSSVLHSTQDLFLPFFSNVTWFHAIHVSGTN | |
| | | | GTKRFDNPVLPFNDGVYFASTEKSNIIRGWIFGTTLDSKTQSLLIVNNATNVVIKVCEFQFCNDPFLGVY | |
| | | | YHKNNKSWMESEFRVYSSANNCTFEYVSQPFLMDLEGKQGNFKNLREFVFKNIDGYFKIYSKHTPINL | |
| | | | VRDLPQGFSALEPLVDLPIGINITRFQTLLALHRSYLTPGDSSSGWTAGAAAYYVGYLQPRTFLLKYNE | |
| | | | NGTITDAVDCALDPLSETKCTLKSFTVEKGIYQTSNFRVQPTESIVRFPNITNLCPFGEVFNATKFPSVY | |
| | | | AWERKKISNCVADYSVLYNSTFFSTFKCYGVSATKLNDLCFSNVYADSFVVKGDDVRQIAPGQTGVIA | |
| | | | DYNYKLPDDFMGCVLAWNTRNIDATSTGNYNYKYRYLRHGKLRPFERDISNVPFSPDGKPCTPPALN | |
| | | | CYWPLNDYGFYTTTGIGYQPYRVVVLSFELLNAPATVCGPKKSTNLVKNKCVNFNFNGLTGTGVLTES | |
| | | | NKKFLPFQQFGRDIADTTDAVRDPQTLEILDITPCSFGGVSVITPGTNTSNQVAVLYQDVNCTEVPVAIH | |
| | | | ADQLTPTWRVYSTGSNVFQTRAGCLIGAEHVNNSYECDIPIGAGICASYQTQTNSPGSASSVASQSIIA | |
| | | | YTMSLGAENSVAYSNNSIAIPTNFTISVTTEILPVSMTKTSVDCTMYICGDSTECSNLLLQYGSFCTQLN | |
| | | | RALTGIAVEQDKNTQEVFAQVKQIYKTPPIKDFGGFNFSQILPDPSKPSKRSFIEDLLFNKVTLADAGFIK | |
| | | | QYGDCLGDIAARDLICAQKFNGLTVLPPLLTDEMIAQYTSALLAGTITSGWTFGAGAALQIPFAMQMAY | |
| | | | RFNGIGVTQNVLYENQKLIANQFNSAIGKIQDSLSSTASALGKLQDVVNQNAQALNTLVKQLSSNFGAI | |
| | | | SSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDVVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP656 | (R)-mosaic-SpFN | MER5 (BatCoV-Ita1 206645- | QGVECDFSKLFKAAPPQIYNFSRLVFTNCNYNLTKLLSLFHVSEFSCHQVSPSALASGCYSSLTVDYFA | 293 |
| | | 40)_mosaicSpFN with SARS- | YPLYLASYLQQGSTGEIAQYNYKQDFSNPTCRILASVPANVSIPKPDKYIWLSQCYSFSAYSGDVPHYV | |
| | | 1 replacing the original | LPGQYTPCLYLTSSGFDNSYQTNRDFQNKMAATGVISSMTDNLQMAFVISVQYGTDTNSVCPMGSG | |
| | | RBD | GSGSSQCVNLTTRTQLPPAYTNSFTRGVYYPDKVFRSSVLHSTQDLFLPFFSNVTWFHAIHVSGTNGT | |
| | | | KRFDNPVLPFNDGVYFASTEKSNIIRGWIFGTTLDSKTQSLLIVNNATNVVIKVCEFQFCNDPFLGVYYH | |
| | | | KNNKSWMESEFRVYSSANNCTFEYVSQPFLMDLEGKQGNFKNLREFVFKNIDGYFKIYSKHTPINLVR | |
| | | | DLPQGFSALEPLVDLPIGINITRFQTLLALHRSYLTPGDSSSGWTAGAAAYYVGYLQPRTFLLKYNENG | |
| | | | TITDAVDCALDPLSETKCTLKSFTVEKGIYQTSNFRVQPTESIVRFPNITNLCPFGEVFNATKFPSVYAW | |
| | | | ERKKISNCVADYSVLYNSTFFSTFKCYGVSATKLNDLCFSNVYADSFVVKGDDVRQIAPGQTGVIADYN | |
| | | | YKLPDDFMGCVLAWNTRNIDATSTGNYNYKYRYLRHGKLRPFERDISNVPFSPDGKPCTPPALNCYW | |
| | | | PLNDYGFYTTTGIGYQPYRVVVLSFELLNAPATVCGPKKSTNLVKNKCVNFNFNGLTGTGVLTESNKK | |
| | | | FLPFQQFGRDIADTTDAVRDPQTLEILDITPCSFGGVSVITPGTNTSNQVAVLYQDVNCTEVPVAIHAD | |
| | | | QLTPTWRVYSTGSNVFQTRAGCLIGAEHVNNSYECDIPIGAGICASYQTQTNSPGSASSVASQSIIAYT | |
| | | | MSLGAENSVAYSNNSIAIPTNFTISVTTEILPVSMTKTSVDCTMYICGDSTECSNLLLQYGSFCTQLNRA | |
| | | | LTGIAVEQDKNTQEVFAQVKQIYKTPPIKDFGGFNFSQILPDPSKPSKRSFIEDLLFNKVTLADAGFIKQY | |
| | | | GDCLGDIAARDLICAQKFNGLTVLPPLLTDEMIAQYTSALLAGTITSGWTFGAGAALQIPFAMQMAYRF | |
| | | | NGIGVTQNVLYENQKLIANQFNSAIGKIQDSLSSTASALGKLQDVVNQNAQALNTLVKQLSSNFGAISS | |
| | | | VLNDILSRLDPPEAEVQIDRLITGRLQSLQTYVTQQLIRAAEIRASANLAATKMSECVLGQSKRVDFCGK | |
| | | | GYHLMSFPQSAPHGVVFLHVTYVPAQEKNFTTAPAICHDGKAHFPREGVFVSNGTHWFVTQRNFYEP | |
| | | | QIITTDNTFVSGNCDVVIGIVNNTVYDPLQSELDSIKEELDKIHKNGSGGSGDIIKLLNEQVNKEMQSSNL | |
| | | | YMSMSSWCYTHSLDGAGLFLFDHAAEEYEHAKKLIIFLNENNVPVQLTSISAPEHKFEGLTQIFQKAYE | |
| | | | HEQHISESINNIVDHAIKSKDHATFNFLQWYVAEQHEEEVLFKDILDKIELIGNENHGLYLADQYVKGIAK | |
| | | | SRKSGS | |
| pCoVP657 | (R)-mosaic-SpFN | HKU5 mosaicSpFN with | TTQECDFTPMLTGTPPPIYNFKRLVFTNCNYNLTKLLSLFQVSEFSCHQVSPSSLATGCYSSLTVDYFA | 294 |
| | | SARS-1 replacing the | YSTDMSSYLQPGSAGAIVQFNYKQDFSNPTCRVLATVPQNLTTITKPSNYAYLTECYKTSAYGKNYLY | |
| | | original RBD | NAPGAYTPCLSLASRGFSTKYQSHSDGELTTTGYIYPVTGNLQMAFIISVQYGTDTNSVCPMGSGGSG | |
| | | | SSQCVNLTTRTQLPPAYTNSFTRGVYYPDKVFRSSVLHSTQDLFLPFFSNVTWFHAIHVSGTNGTKRF | |
| | | | DNPVLPFNDGVYFASTEKSNIIRGWIFGTTLDSKTQSLLIVNNATNVVIKVCEFQFCNDPFLGVYYHKNN | |
| | | | KSWMESEFRVYSSANNCTFEYVSQPFLMDLEGKQGNFKNLREFVFKNIDGYFKIYSKHTPINLVRDLP | |
| | | | QGFSALEPLVDLPIGINITRFQTLLALHRSYLTPGDSSSGWTAGAAAYYVGYLQPRTFLLKYNENGTITD | |
| | | | AVDCALDPLSETKCTLKSFTVEKGIYQTSNFRVQPTESIVRFPNITNLCPFGEVFNATKFPSVYAWERK | |
| | | | KISNCVADYSVLYNSTFFSTFKCYGVSATKLNDLCFSNVYADSFVVKGDDVRQIAPGQTGVIADYNYKL | |
| | | | PDDFMGCVLAWNTRNIDATSTGNYNYKYRYLRHGKLRPFERDISNVPFSPDGKPCTPPALNCYWPLN | |
| | | | DYGFYTTTGIGYQPYRVVVLSFELLNAPATVCGPKKSTNLVKNKCVNFNFNGLTGTGVLTESNKKFLP | |
| | | | FQQFGRDIADTTDAVRDPQTLEILDITPCSFGGVSVITPGTNTSNQVAVLYQDVNCTEVPVAIHADQLT | |
| | | | PTWRVYSTGSNVFQTRAGCLIGAEHVNNSYECDIPIGAGICASYQTQTNSPGSASSVASQSIIAYTMSL | |
| | | | GAENSVAYSNNSIAIPTNFTISVTTEILPVSMTKTSVDCTMYICGDSTECSNLLLQYGSFCTQLNRALTGI | |
| | | | AVEQDKNTQEVFAQVKQIYKTPPIKDFGGFNFSQILPDPSKPSKRSFIEDLLFNKVTLADAGFIKQYGDC | |
| | | | LGDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP658 | (RR)-mosaic-SpFN | RBD from Omicron BA.2.12.1 | NITNLCPFDEVFNATRFASVYAWNRKRISNCVADYSVLYNFAPFFAFKCYGVSPTKLNDLCFTNVYADS | 295 |
| | | & RBD from Omicron | FVIRGNEVSQIAPGQTGNIADYNYKLPDDFTGCVIAWNSNKLDSKVGGNYNYQYRLFRKSNLKPFERD | |
| | | BA.4/5 mosaicSpFN with | ISTEIYQAGNKPCNGVAGFNCYFPLRSYGFRPTYGVGHQPYRVVVLSFELLHAPATVCGPGSGGSGNI | |
| | | SARS-1 replacing the | TNLCPFDEVFNATRFASVYAWNRKRISNCVADYSVLYNFAPFFAFKCYGVSPTKLNDLCFTNVYADSF | |
| | | original RBD | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP659 | (RR)-mosaic-SpFN | RBD from NC_014470. | NITQLCPFNEVFNITSFPSVYAWERMRITNCVADYSVLYNSSASFSTFQCYGVSPTKLNDLCFSSVYAD | 296 |
| | | BM4831.BGR.2008 & RBD from | YFVVKGDDVRQIAPAQTGVIADYNYKLPDDFTGCVIAWNTNSLDSSNEFFYRRFRHGKIKPYGRDLSN | |
| | | Omicron BA.4/5_mosaicSpFN | VLFNPSGGTCSAEGLNCYKPLASYGFTQSSGIGFQPYRVVVLSFELLNAPATVCGPGSGGSGNITNLC | |
| | | with SARS-1 replacing the | PFDEVFNATRFASVYAWNRKRISNCVADYSVLYNFAPFFAFKCYGVSPTKLNDLCFTNVYADSFVIRG | |
| | | original RBD | NEVSQIAPGQTGNIADYNYKLPDDFTGCVIAWNSNKLDSKVGGNYNYRYRLFRKSNLKPFERDISTEIY | |
| | | | QAGNKPCNGVAGVNCYFPLQSYGFRPTYGVGHQPYRVVVLSFELLHAPATVCGPGSGGSGSSQCV | |
| | | | NLTTRTQLPPAYTNSFTRGVYYPDKVFRSSVLHSTQDLFLPFFSNVTWFHAIHVSGTNGTKRFDNPVL | |
| | | | PFNDGVYFASTEKSNIIRGWIFGTTLDSKTQSLLIVNNATNVVIKVCEFQFCNDPFLGVYYHKNNKSWM | |
| | | | ESEFRVYSSANNCTFEYVSQPFLMDLEGKQGNFKNLREFVFKNIDGYFKIYSKHTPINLVRDLPQGFSA | |
| | | | LEPLVDLPIGINITRFQTLLALHRSYLTPGDSSSGWTAGAAAYYVGYLQPRTFLLKYNENGTITDAVDCA | |
| | | | LDPLSETKCTLKSFTVEKGIYQTSNFRVQPTESIVRFPNITNLCPFGEVFNATKFPSVYAWERKKISNCV | |
| | | | ADYSVLYNSTFFSTFKCYGVSATKLNDLCFSNVYADSFVVKGDDVRQIAPGQTGVIADYNYKLPDDFM | |
| | | | GCVLAWNTRNIDATSTGNYNYKYRYLRHGKLRPFERDISNVPFSPDGKPCTPPALNCYWPLNDYGFY | |
| | | | TTTGIGYQPYRVVVLSFELLNAPATVCGPKKSTNLVKNKCVNFNFNGLTGTGVLTESNKKFLPFQQFG | |
| | | | RDIADTTDAVRDPQTLEILDITPCSFGGVSVITPGTNTSNQVAVLYQDVNCTEVPVAIHADQLTPTWRV | |
| | | | YSTGSNVFQTRAGCLIGAEHVNNSYECDIPIGAGICASYQTQTNSPGSASSVASQSIIAYTMSLGAENS | |
| | | | VAYSNNSIAIPTNFTISVTTEILPVSMTKTSVDCTMYICGDSTECSNLLLQYGSFCTQLNRALTGIAVEQD | |
| | | | KNTQEVFAQVKQIYKTPPIKDFGGFNFSQILPDPSKPSKRSFIEDLLFNKVTLADAGFIKQYGDCLGDIA | |
| | | | ARDLICAQKFNGLTVLPPLLTDEMIAQYTSALLAGTITSGWTFGAGAALQIPFAMQMAYRFNGIGVTQN | |
| | | | VLYENQKLIANQFNSAIGKIQDSLSSTASALGKLQDVVNQNAQALNTLVKQLSSNFGAISSVLNDILSRL | |
| | | | DPPEAEVQIDRLITGRLQSLQTYVTQQLIRAAEIRASANLAATKMSECVLGQSKRVDFCGKGYHLMSFP | |
| | | | QSAPHGVVFLHVTYVPAQEKNFTTAPAICHDGKAHFPREGVFVSNGTHWFVTQRNFYEPQIITTDNTF | |
| | | | VSGNCDWVIGIVNNTVYDPLQSELDSIKEELDKIHKNGSGGSGDIIKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESI | |
| | | | NNIVDHAIKSKDHATFNFLQWYVAEQHEEEVLFKDILDKIELIGNENHGLYLADQYVKGIAKSRKSGS | |
| pCoVP660 | (RR)-mosaic-SpFN | BM48-31 RBD-T498W & RBD | NITQLCPFNEVFNITSFPSVYAWERMRITNCVADYSVLYNSSASFSTFQCYGVSPTKLNDLCFSSVYAD | 297 |
| | | from Omicron BA.4/5_ | YFVVKGDDVRQIAPAQTGVIADYNYKLPDDFTGCVIAWNTNSLDSSNEFFYRRFRHGKIKPYGRDLSN | |
| | | mosaicSpFN with SARS-1 | VLFNPSGGTCSAEGLNCYKPLASYGFWQSSGIGFQPYRVVVLSFELLNAPATVCGPGSGGSGNITNL | |
| | | replacing the original RBD | CPFDEVFNATRFASVYAWNRKRISNCVADYSVLYNFAPFFAFKCYGVSPTKLNDLCFTNVYADSFVIR | |
| | | | GNEVSQIAPGQTGNIADYNYKLPDDFTGCVIAWNSNKLDSKVGGNYNYRYRLFRKSNLKPFERDISTEI | |
| | | | YQAGNKPCNGVAGVNCYFPLQSYGFRPTYGVGHQPYRVVVLSFELLHAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATKFPSVYAWERKKISN | |
| | | | CVADYSVLYNSTFFSTFKCYGVSATKLNDLCFSNVYADSFVVKGDDVRQIAPGQTGVIADYNYKLPDD | |
| | | | FMGCVLAWNTRNIDATSTGNYNYKYRYLRHGKLRPFERDISNVPFSPDGKPCTPPALNCYWPLNDYG | |
| | | | FYTTTGIGYQPYRVVVLSFELLNAPATVCGPKKSTNLVKNKCVNFNFNGLTGTGVLTESNKKFLPFQQ | |
| | | | FGRDIADTTDAVRDPQTLEILDITPCSFGGVSVITPGTNTSNQVAVLYQDVNCTEVPVAIHADQLTPTW | |
| | | | RVYSTGSNVFQTRAGCLIGAEHVNNSYECDIPIGAGICASYQTQTNSPGSASSVASQSIIAYTMSLGAE | |
| | | | NSVAYSNNSIAIPTNFTISVTTEILPVSMTKTSVDCTMYICGDSTECSNLLLQYGSFCTQLNRALTGIAVE | |
| | | | QDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP661 | (RR)-mosaic-SpFN | BM48-31 RBD-A493Y-T498W & | NITQLCPFNEVFNITSFPSVYAWERMRITNCVADYSVLYNSSASFSTFQCYGVSPTKLNDLCFSSVYAD | 298 |
| | | RBD from Omicron | YFVVKGDDVRQIAPAQTGVIADYNYKLPDDFTGCVIAWNTNSLDSSNEFFYRRFRHGKIKPYGRDLSN | |
| | | BA.4/5_mosaicSpFN with | VLFNPSGGTCSAEGLNCYKPLYSYGFWQSSGIGFQPYRVVVLSFELLNAPATVCGPGSGGSGNITNL | |
| | | SARS-1 replacing the | CPFDEVFNATRFASVYAWNRKRISNCVADYSVLYNFAPFFAFKCYGVSPTKLNDLCFTNVYADSFVIR | |
| | | original RBD | GNEVSQIAPGQTGNIADYNYKLPDDFTGCVIAWNSNKLDSKVGGNYNYRYRLFRKSNLKPFERDISTEI | |
| | | | YQAGNKPCNGVAGVNCYFPLQSYGFRPTYGVGHQPYRVVVLSFELLHAPATVCGPGSGGSGSSQC | |
| | | | VNLTTRTQLPPAYTNSFTRGVYYPDKVFRSSVLHSTQDLFLPFFSNVTWFHAIHVSGTNGTKRFDNPV | |
| | | | LPFNDGVYFASTEKSNIIRGWIFGTTLDSKTQSLLIVNNATNVVIKVCEFQFCNDPFLGVYYHKNNKSW | |
| | | | MESEFRVYSSANNCTFEYVSQPFLMDLEGKQGNFKNLREFVFKNIDGYFKIYSKHTPINLVRDLPQGF | |
| | | | SALEPLVDLPIGINITRFQTLLALHRSYLTPGDSSSGWTAGAAAYYVGYLQPRTFLLKYNENGTITDAVD | |
| | | | CALDPLSETKCTLKSFTVEKGIYQTSNFRVQPTESIVRFPNITNLCPFGEVFNATKFPSVYAWERKKISN | |
| | | | CVADYSVLYNSTFFSTFKCYGVSATKLNDLCFSNVYADSFVVKGDDVRQIAPGQTGVIADYNYKLPDD | |
| | | | FMGCVLAWNTRNIDATSTGNYNYKYRYLRHGKLRPFERDISNVPFSPDGKPCTPPALNCYWPLNDYG | |
| | | | FYTTTGIGYQPYRVWVLSFELLNAPATVCGPKKSTNLVKNKCVNFNFNGLTGTGVLTESNKKFLPFQQ | |
| | | | FGRDIADTTDAVRDPQTLEILDITPCSFGGVSVITPGTNTSNQVAVLYQDVNCTEVPVAIHADQLTPTW | |
| | | | RVYSTGSNVFQTRAGCLIGAEHVNNSYECDIPIGAGICASYQTQTNSPGSASSVASQSIIAYTMSLGAE | |
| | | | NSVAYSNNSIAIPTNFTISVTTEILPVSMTKTSVDCTMYICGDSTECSNLLLQYGSFCTQLNRALTGIAVE | |
| | | | QDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP662 | (RR)-mosaic-SpFN | RBD from KC881005.RsSHC014 | NITNLCPFGEVFNATTFPSVYAWERKRISNCVADYSVLYNSTSFSTFKCYGVSATKLNDLCFSNVYADS | 299 |
| | | & RBD from Omicron | FVVKGDDVRQIAPGQTGVIADYNYKLPDDFLGCVLAWNTNSKDSSTSGNYNYLYRWVRRSKLNPYER | |
| | | BA.4/5 mosaicSpFN with | DLSNDIYSPGGQSCSAVGPNCYNPLRPYGFFTTAGVGHQPYRVVVLSFELLNAPATVCGPGSGGSG | |
| | | SARS-1 replacing the | NITNLCPFDEVFNATRFASVYAWNRKRISNCVADYSVLYNFAPFFAFKCYGVSPTKLNDLCFTNVYADS | |
| | | original RBD | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP663 | (RR)-mosaic-SpFN | RBD from KC881007.Bat.WIV1 | NITNLCPFGEVFNATTFPSVYAWERKRISNCVADYSVLYNSTSFSTFKCYGVSATKLNDLCFSNVYADS | 300 |
| | | & RBD from Omicron | FVVKGDDVRQIAPGQTGVIADYNYKLPDDFTGCVLAWNTRNIDATQTGNYNYKYRSLRHGKLRPFER | |
| | | BA.4/5_mosaicSpFN with | DISNVPFSPDGKPCTPPAFNCYWPLNDYGFYITNGIGYQPYRVVVLSFELLNAPATVCGPGSGGSGNI | |
| | | SARS-1 replacing the | TNLCPFDEVFNATRFASVYAWNRKRISNCVADYSVLYNFAPFFAFKCYGVSPTKLNDLCFTNVYADSF | |
| | | original RBD | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP664 | (RR)-mosaic-SpFN | RBD from AVP78042_ | NITNVCPFHKVFNATRFPSVYAWERTKISDCIADYTVFYNSTSFSTFKCYGVSPSKLIDLCFTSVYADTF | 301 |
| | | BatSARSlikecoronavirus & | LIRFSEVRQVAPGQTGVIADYNYKLPDDFTGCVIAWNTAKQDTGHYFYRSHRSTKLKPFERDLSSDEN | |
| | | RBD from Omicron BA.4/5_ | GVRTLSTYDFNPNVPLEYQATRVVVLSFELLNAPATVCGPGSGGSGNITNLCPFDEVFNATRFASVYA | |
| | | mosaicSpFN with SARS-1 | WNRKRISNCVADYSVLYNFAPFFAFKCYGVSPTKLNDLCFTNVYADSFVIRGNEVSQIAPGQTGNIADY | |
| | | replacing the original RBD | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP665 | (RR)-mosaic-SpFN | RBD from MZ937001_ | NITNLCPFGEVFNATTFASVYAWNRKRISNCVADYSVLYNSTSFSTFKCYGVSPTKLNDLCFTNVYADS | 302 |
| | | BANAL20103_Laos_2020 & RBD | FVVRGDEVRQIAPGQTGKIADYNYKLPDDFTGCVIAWNSNNLDSKVGGNYNYLYRLFRKSNLKPFERD | |
| | | from Omicron BA.4/5_ | ISTEIYQAGSTPCNGVEGFNCYFPLQSYGFHPTNGVGYQPYRVVVLSFELLNAPATVCGPGSGGSGNI | |
| | | mosaicSpFN with SARS-1 | TNLCPFDEVFNATRFASVYAWNRKRISNCVADYSVLYNFAPFFAFKCYGVSPTKLNDLCFTNVYADSF | |
| | | replacing the original RBD | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP666 | (RR)-mosaic-SpFN | RBD from MN996532.2013. | NITNLCPFGEVFNATTFASVYAWNRKRISNCVADYSVLYNSTSFSTFKCYGVSPTKLNDLCFTNVYADS | 303 |
| | | Bat.RaTG13 & RBD from | FVITGDEVRQIAPGQTGKIADYNYKLPDDFTGCVIAWNSKHIDAKEGGNFNYLYRLFRKANLKPFERDI | |
| | | Omicron BA.4/5_mosaicSpFN | STEIYQAGSKPCNGQTGLNCYYPLYRYGFYPTDGVGHQPYRVVVLSFELLNAPATVCGPGSGGSGNI | |
| | | with SARS-1 replacing the | TNLCPFDEVFNATRFASVYAWNRKRISNCVADYSVLYNFAPFFAFKCYGVSPTKLNDLCFTNVYADSF | |
| | | original RBD | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVWVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP667 | (RR)-mosaic-SpFN | RBD from MZ937004_ | NITNFCPFDKVFNATRFPNVYAWQRTKISDCIADYTVLYNSTSFSTFKCYGVSPSKLIDLCFTSVYADTF | 304 |
| | | BANAL20247_Laos_2020 & RBD | LIRFSEVRQIAPGETGVIADYNYKLPDDFTGCVLAWNTAQQDIGSYFYRSHRAVKLKPFERDLSSDEN | |
| | | from Omicron BA.4/5_ | GVRTLSTYDFNPNVPLDYQATRVVVLSFELLNAPATVCGPGSGGSGNITNLCPFDEVFNATRFASVYA | |
| | | mosaicSpFN with SARS-1 | WNRKRISNCVADYSVLYNFAPFFAFKCYGVSPTKLNDLCFTNVYADSFVIRGNEVSQIAPGQTGNIADY | |
| | | replacing the original RBD | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP668 | (RR)-mosaic-SpFN | BANAL-20-247 RBD-N498W & | NITNFCPFDKVFNATRFPNVYAWQRTKISDCIADYTVLYNSTSFSTFKCYGVSPSKLIDLCFTSVYADTF | 305 |
| | | RBD from Omicron BA.4/5_ | LIRFSEVRQIAPGETGVIADYNYKLPDDFTGCVLAWNTAQQDIGSYFYRSHRAVKLKPFERDLSSDEN | |
| | | mosaicSpFN with SARS-1 | GVRTLSTYDFWPNVPLDYQATRVVVLSFELLNAPATVCGPGSGGSGNITNLCPFDEVFNATRFASVYA | |
| | | replacing the original RBD | WNRKRISNCVADYSVLYNFAPFFAFKCYGVSPTKLNDLCFTNVYADSFVIRGNEVSQIAPGQTGNIADY | |
| | | | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | \|GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP669 | (RR)-mosaic-SpFN | BANAL-20-247 RBD-S493Y- | NITNFCPFDKVFNATRFPNVYAWQRTKISDCIADYTVLYNSTSFSTFKCYGVSPSKLIDLCFTSVYADTF | 306 |
| | | N498W & RBD from Omicron | LIRFSEVRQIAPGETGVIADYNYKLPDDFTGCVLAWNTAQQDIGSYFYRSHRAVKLKPFERDLSSDEN | |
| | | BA.4/5 mosaicSpFN with | GVRTLYTYDFWPNVPLDYQATRVVVLSFELLNAPATVCGPGSGGSGNITNLCPFDEVFNATRFASVYA | |
| | | SARS-1 replacing the | WNRKRISNCVADYSVLYNFAPFFAFKCYGVSPTKLNDLCFTNVYADSFVIRGNEVSQIAPGQTGNIADY | |
| | | original RBD | NYKLPDDFTGCVIAWNSNKLDSKVGGNYNYRYRLFRKSNLKPFERDISTEIYQAGNKPCNGVAGVNC | |
| | | | YFPLQSYGFRPTYGVGHQPYRVVVLSFELLHAPATVCGPGSGGSGSSQCVNLTTRTQLPPAYTNSFT | |
| | | | RGVYYPDKVFRSSVLHSTQDLFLPFFSNVTWFHAIHVSGTNGTKRFDNPVLPFNDGVYFASTEKSNIIR | |
| | | | GWIFGTTLDSKTQSLLIVNNATNVVIKVCEFQFCNDPFLGVYYHKNNKSWMESEFRVYSSANNCTFEY | |
| | | | VSQPFLMDLEGKQGNFKNLREFVFKNIDGYFKIYSKHTPINLVRDLPQGFSALEPLVDLPIGINITRFQTL | |
| | | | LALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP670 | (RR)-mosaic-SpFN | RBD from ATO98145_ | NITNLCPFDAVFNATRFPSVYAWERVKISNCVADYTAFYNSTSFSTFKCYGVSPSKLIDLCFTSVYADT | 307 |
| | | BatSARSlikecoronavirus & | FLIRFSEVRQVAPGETGVIADYNYKLPDDFTGCVIAWNTAKQDVGSYFYRSHRSSKLKPFERDLSSDE | |
| | | RBD from Omicron BA.4/5_ | NGVRTLSTYDFNPNVPLDYQATRVVVLSFELLNAPATVCGPGSGGSGNITNLCPFDEVFNATRFASVY | |
| | | mosaicSpFN with SARS-1 | AWNRKRISNCVADYSVLYNFAPFFAFKCYGVSPTKLNDLCFTNVYADSFVIRGNEVSQIAPGQTGNIAD | |
| | | replacing the original RBD | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP671 | (RR)-mosaic-SpFN | RBD from JX993987.2011. | NITNRCPFDKVFNATRFPSVYAWERTKISDCVADYTVLYNSTSFSTFKCYGVSPSKLIDLCFTSVYADT | 308 |
| | | BatRp.Shaanxi2011 & RBD | FLIRSSEVRQVAPGETGVIADYNYKLPDDFTGCVIAWNTANQDQGQYYYRSSRKEKLKPFERDLSSDE | |
| | | from Omicron BA.4/5_ | NGVYTLSTYDFYPSVPLDYQATRVVVLSFELLNAPATVCGPGSGGSGNITNLCPFDEVFNATRFASVY | |
| | | mosaicSpFN with SARS-1 | AWNRKRISNCVADYSVLYNFAPFFAFKCYGVSPTKLNDLCFTNVYADSFVIRGNEVSQIAPGQTGNIAD | |
| | | replacing the original RBD | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP672 | (RR)-mosaic-SpFN | RBD from KJ473812. | NITNLCPFDKVFNATRFPSVYAWERTKISDCVADYTVFYNSTSFSTFNCYGVSPSKLIDLCFTSVYADT | 309 |
| | | BtRfBetaCoV.HeB2013 & RBD | FLIRFSEVRQVAPGQTGVIADYNYKLPDDFTGCVIAWNTAKQDVGSYFYRSHRSSKLKPFERDLSSEE | |
| | | from Omicron BA.4/5_ | NGVRTLSTYDFNQYVPLEYQATRVVVLSFELLNAPATVCGPGSGGSGNITNLCPFDEVFNATRFASVY | |
| | | mosaicSpFN with SARS-1 | AWNRKRISNCVADYSVLYNFAPFFAFKCYGVSPTKLNDLCFTNVYADSFVIRGNEVSQIAPGQTGNIAD | |
| | | replacing the original RBD | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP673 | (RR)-mosaic-SpFN | RBD from DQ071615.2003. | NITNRCPFDKVFNATRFPNVYAWERTKISDCVADYTVLYNSTSFSTFKCYGVSPSKLIDLCFTSVYADT | 310 |
| | | Bat.Rp3 & RBD from Omicron | FLIRSSEVRQVAPGETGVIADYNYKLPDDFTGCVIAWNTAKQDQGQYYYRSHRKTKLKPFERDLSSDE | |
| | | BA.4/5_mosaicSpFN with | NGVRTLSTYDFYPSVPVAYQATRVVVLSFELLNAPATVCGPGSGGSGNITNLCPFDEVFNATRFASVY | |
| | | SARS-1 replacing the | AWNRKRISNCVADYSVLYNFAPFFAFKCYGVSPTKLNDLCFTNVYADSFVIRGNEVSQIAPGQTGNIAD | |
| | | original RBD | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP674 | (RR)-mosaic-SpFN | RBD from ACU31032_Rs_ | NITNRCPFDKVFNASRFPNVYAWERTKISDCVADYTVLYNSTSFSTFKCYGVSPSKLIDLCFTSVYADT | 311 |
| | | 672_2006 & RBD from Omicron | FLIRSSEVRQVAPGETGVIADYNYKLPDDFTGCVIAWNTAKQDQGQYYYRSSRKTKLKPFERDLTSDE | |
| | | BA.4/5_mosaicSpFN with | NGVRTLSTYDFYPNVPIEYQATRVVVLSFELLNAPATVCGPGSGGSGNITNLCPFDEVFNATRFASVY | |
| | | SARS-1 replacing the | AWNRKRISNCVADYSVLYNFAPFFAFKCYGVSPTKLNDLCFTNVYADSFVIRGNEVSQIAPGQTGNIAD | |
| | | original RBD | YNYKLPDDFTGCVIAWNSNKLDSKVGGNYNYRYRLFRKSNLKPFERDISTEIYQAGNKPCNGVAGVN | |
| | | | CYFPLQSYGFRPTYGVGHQPYRVVVLSFELLHAPATVCGPGSGGSGSSQCVNLTTRTQLPPAYTNSF | |
| | | | TRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNWVIKVCEFQFCNDPFLGVYYHKNNKSWMESEFRVYSSANNCTFE | |
| | | | YVSQPFLMDLEGKQGNFKNLREFVFKNIDGYFKIYSKHTPINLVRDLPQGFSALEPLVDLPIGINITRFQT | |
| | | | LLALHRSYLTPGDSSSGWTAGAAAYYVGYLQPRTFLLKYNENGTITDAVDCALDPLSETKCTLKSFTVE | |
| | | | KGIYQTSNFRVQPTESIVRFPNITNLCPFGEVFNATKFPSVYAWERKKISNCVADYSVLYNSTFFSTFK | |
| | | | CYGVSATKLNDLCFSNVYADSFVVKGDDVRQIAPGQTGVIADYNYKLPDDFMGCVLAWNTRNIDATST | |
| | | | GNYNYKYRYLRHGKLRPFERDISNVPFSPDGKPCTPPALNCYWPLNDYGFYTTTGIGYQPYRVVVLSF | |
| | | | ELLNAPATVCGPKKSTNLVKNKCVNFNFNGLTGTGVLTESNKKFLPFQQFGRDIADTTDAVRDPQTLEI | |
| | | | LDITPCSFGGVSVITPGTNTSNQVAVLYQDVNCTEVPVAIHADQLTPTWRVYSTGSNVFQTRAGCLIG | |
| | | | AEHVNNSYECDIPIGAGICASYQTQTNSPGSASSVASQSIIAYTMSLGAENSVAYSNNSIAIPTNFTISVT | |
| | | | TEILPVSMTKTSVDCTMYICGDSTECSNLLLQYGSFCTQLNRALTGIAVEQDKNTQEVFAQVKQIYKTP | |
| | | | PIKDFGGFNFSQILPDPSKPSKRSFIEDLLFNKVTLADAGFIKQYGDCLGDIAARDLICAQKFNGLTVLPP | |
| | | | LLTDEMIAQYTSALLAGTITSGWTFGAGAALQIPFAMQMAYRFNGIGVTQNVLYENQKLIANQFNSAIG | |
| | | | KIQDSLSSTASALGKLQDVVNQNAQALNTLVKQLSSNFGAISSVLNDILSRLDPPEAEVQIDRLITGRLQ | |
| | | | SLQTYVTQQLIRAAEIRASANLAATKMSECVLGQSKRVDFCGKGYHLMSFPQSAPHGVVFLHVTYVPA | |
| | | | QEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP675 | (RR)-mosaic-SpFN | RBD from MZ081380_bat_ | NITNLCPFSQVFNATRFPSVYAWTRERISNCIADYSVLYNSTSFSTFRCYGVSPTKLNDLCFSNVYADS | 312 |
| | | Yunnan_RsYN04_2020 & RBD | MVVRGDEVRQIAPSQTGVIADYNYKLPDDFTGCVIAWNSKAKDENGQYFYRLFRKSKLLPFQRDVSN | |
| | | from Omicron BA.4/ _ | VTYGSGKNDGCNPSEADCYWPLLKYGFTSSVSQDYQPYRVVVLSFELLNAPATVCGPGSGGSGNIT | |
| | | mosaicSpFN with SARS-1 | NLCPFDEVFNATRFASVYAWNRKRISNCVADYSVLYNFAPFFAFKCYGVSPTKLNDLCFTNVYADSFVI | |
| | | replacing the original RBD | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP676 | (RR)-mosaic-SpFN | RsYN04 RBD-T498W & RBD | NITNLCPFSQVFNATRFPSVYAWTRERISNCIADYSVLYNSTSFSTFRCYGVSPTKLNDLCFSNVYADS | 313 |
| | | from Omicron BA.4/5_ | MVVRGDEVRQIAPSQTGVIADYNYKLPDDFTGCVIAWNSKAKDENGQYFYRLFRKSKLLPFQRDVSN | |
| | | mosaicSpFN with SARS-1 | VTYGSGKNDGCNPSEADCYWPLLKYGFWSSVSQDYQPYRVVVLSFELLNAPATVCGPGSGGSGNIT | |
| | | replacing the original RBD | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP677 | (RR)-mosaic-SpFN | RsYN04 RBD-L493Y-T498W & | NITNLCPFSQVFNATRFPSVYAWTRERISNCIADYSVLYNSTSFSTFRCYGVSPTKLNDLCFSNVYADS | 314 |
| | | RBD from Omicron BA.4/5_ | MVVRGDEVRQIAPSQTGVIADYNYKLPDDFTGCVIAWNSKAKDENGQYFYRLFRKSKLLPFQRDVSN | |
| | | mosaicSpFN with SARS-1 | VTYGSGKNDGCNPSEADCYWPLYKYGFWSSVSQDYQPYRVVVLSFELLNAPATVCGPGSGGSGNIT | |
| | | replacing the original RBD | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP678 | (RR)-mosaic-SpFN | Khosta2-MZ190138 RBD & RBD | NMTNICPFDQVFNKTQFPSVYAWERVRISDCVSDYTVLYNSSASFSTFKCYGVSPTKLNDLCFSGVYA | 315 |
| | | from Omicron BA.4/5_ | DYFWVKGDHVHQIAPGQTGVIADYNYKLPSEFVGCILAWNTRTIDSKRGFYYRLFRHGNIRPYERDTSN | |
| | | mosaicSpFN with SARS-1 | VPYNAAGGTCNQPGTHNCYEPLQDYGFTSTSGVGYQPFRVVVLSFELLNAPATVCGPGSGGSGNIT | |
| | | replacing the original RBD | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVWVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP679 | (RR)-mosaic-SpFN | Khosta-2 RBD-T498W & RBD | NMTNICPFDQVFNKTQFPSVYAWERVRISDCVSDYTVLYNSSASFSTFKCYGVSPTKLNDLCFSGVYA | 316 |
| | | from Omicron BA.4/5_ | DYFWVKGDHVHQIAPGQTGVIADYNYKLPSEFVGCILAWNTRTIDSKRGFYYRLFRHGNIRPYERDTSN | |
| | | mosaicSpFN with SARS-1 | VPYNAAGGTCNQPGTHNCYEPLQDYGFWSTSGVGYQPFRVVVLSFELLNAPATVCGPGSGGSGNIT | |
| | | replacing the original RBD | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP680 | (RR)-mosaic-SpFN | Khosta-2 RBD-Q493Y-T498W & | NMTNICPFDQVFNKTQFPSVYAWERVRISDCVSDYTVLYNSSASFSTFKCYGVSPTKLNDLCFSGVYA | 317 |
| | | RBD from Omicron | DYFWVKGDHVHQIAPGQTGVIADYNYKLPSEFVGCILAWNTRTIDSKRGFYYRLFRHGNIRPYERDTSN | |
| | | BA.4/5_mosaicSpFN with | VPYNAAGGTCNQPGTHNCYEPLYDYGFWSTSGVGYQPFRVVVLSFELLNAPATVCGPGSGGSGNIT | |
| | | SARS-1 replacing the | NLCPFDEVFNATRFASVYAWNRKRISNCVADYSVLYNFAPFFAFKCYGVSPTKLNDLCFTNVYADSFVI | |
| | | original RBD | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP681 | (RR)-mosaic-SpFN | KY352407_BtKY72_bat_Kenya | NITNLCPFGQVFNASNFPSVYAWERLRISDCVADYAVLYNSSSSFSTFKCYGVSPTKLNDLCFSSVYA | 318 |
| | | & RBD from Omicron | DYFWKGDDVRQIAPAQTGVIADYNYKLPDDFTGCVLAWNTNSVDSKSGNNFYYRLFRHGKIKPYER | |
| | | BA.4/5_mosaicSpFN with | DISNVLYNSAGGTCSSISQLGCYEPLKSYGFTPTVGVGYQPYRVVVLSFELLNAPATVCGPGSGGSGN | |
| | | SARS-1 replacing the | ITNLCPFDEVFNATRFASVYAWNRKRISNCVADYSVLYNFAPFFAFKCYGVSPTKLNDLCFTNVYADSF | |
| | | original RBD | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFWVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP682 | (RR)-mosaic-SpFN | BtKY72 RBD-T498W & RBD | NITNLCPFGQVFNASNFPSVYAWERLRISDCVADYAVLYNSSSSFSTFKCYGVSPTKLNDLCFSSVYA | 319 |
| | | from Omicron BA.4/5_ | DYFWVKGDDVRQIAPAQTGVIADYNYKLPDDFTGCVLAWNTNSVDSKSGNNFYYRLFRHGKIKPYER | |
| | | mosaicSpFN with SARS-1 | DISNVLYNSAGGTCSSISQLGCYEPLKSYGFWPTVGVGYQPYRVVVLSFELLNAPATVCGPGSGGSG | |
| | | replacing the original RBD | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP683 | (RR)-mosaic-SpFN | BtKY72 RBD-K493Y-T498W & | NITNLCPFGQVFNASNFPSVYAWERLRISDCVADYAVLYNSSSSFSTFKCYGVSPTKLNDLCFSSVYA | 320 |
| | | RBD from Omicron BA.4/5_ | DYFWVKGDDVRQIAPAQTGVIADYNYKLPDDFTGCVLAWNTNSVDSKSGNNFYYRLFRHGKIKPYER | |
| | | mosaicSpFN with SARS-1 | DISNVLYNSAGGTCSSISQLGCYEPLYSYGFWPTVGVGYQPYRVVVLSFELLNAPATVCGPGSGGSG | |
| | | replacing the original RBD | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP684 | (RR)-mosaic-SpFN | RaTG15_Ra7909 (RBD) & RBD | NITNLCPFSQVFNATRFPSVYAWTRERISNCIADYSVLYNSTSFSTFRCYGVSPTKLNDLCFSNVYADS | 321 |
| | | from Omicron BA.4/5_ | MVVRGDEVRQIAPSQTGVIADYNYKLPDDFTGCVIAWNSKAKDENGQYFYRLFRKSKLLPFQRDVSN | |
| | | mosaicSpFN with SARS-1 | VTYGSGKNDGCNPSEADCYWPLLKYGFTGSVSQDYQPYRVVVLSFELLNAPATVCGPGSGGSGNIT | |
| | | replacing the original RBD | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP685 | (RR)-mosaic-SpFN | HKU3-1_DQ022305.2005 (RBD) | NITNRCPFDKVFNATRFPNVYAWERTKISDCVADYTVLYNSTSFSTFKCYGVSPSKLIDLCFTSVYADT | 322 |
| | | & RBD from Omicron | FLIRSSEVRQVAPGETGVIADYNYKLPDDFTGCVIAWNTAKHDTGNYYYRSHRKTKLKPFERDLSSDD | |
| | | BA.4/5_mosaicSpFN with | GNGVYTLSTYDFNPNVPVAYQATRVVVLSFELLNAPATVCGPGSGGSGNITNLCPFDEVFNATRFASV | |
| | | SARS-1 replacing the | YAWNRKRISNCVADYSVLYNFAPFFAFKCYGVSPTKLNDLCFTNVYADSFVIRGNEVSQIAPGQTGNIA | |
| | | original RBD | DYNYKLPDDFTGCVIAWNSNKLDSKVGGNYNYRYRLFRKSNLKPFERDISTEIYQAGNKPCNGVAGV | |
| | | | NCYFPLQSYGFRPTYGVGHQPYRVVVLSFELLHAPATVCGPGSGGSGSSQCVNLTTRTQLPPAYTNS | |
| | | | FTRGVYYPDKVFRSSVLHSTQDLFLPFFSNVTWFHAIHVSGTNGTKRFDNPVLPFNDGVYFASTEKSN | |
| | | | IIRGWIFGTTLDSKTQSLLIVNNATNVVIKVCEFQFCNDPFLGVYYHKNNKSWMESEFRVYSSANNCTF | |
| | | | EYVSQPFLMDLEGKQGNFKNLREFVFKNIDGYFKIYSKHTPINLVRDLPQGFSALEPLVDLPIGINITRFQ | |
| | | | TLLALHRSYLTPGDSSSGWTAGAAAYYVGYLQPRTFLLKYNENGTITDAVDCALDPLSETKCTLKSFTV | |
| | | | EKGIYQTSNFRVQPTESIVRFPNITNLCPFGEVFNATKFPSVYAWERKKISNCVADYSVLYNSTFFSTF | |
| | | | KCYGVSATKLNDLCFSNVYADSFVVKGDDVRQIAPGQTGVIADYNYKLPDDFMGCVLAWNTRNIDAT | |
| | | | STGNYNYKYRYLRHGKLRPFERDISNVPFSPDGKPCTPPALNCYWPLNDYGFYTTTGIGYQPYRVVVL | |
| | | | SFELLNAPATVCGPKKSTNLVKNKCVNFNFNGLTGTGVLTESNKKFLPFQQFGRDIADTTDAVRDPQT | |
| | | | LEILDITPCSFGGVSVITPGTNTSNQVAVLYQDVNCTEVPVAIHADQLTPTWRVYSTGSNVFQTRAGCL | |
| | | | IGAEHVNNSYECDIPIGAGICASYQTQTNSPGSASSVASQSIIAYTMSLGAENSVAYSNNSIAIPTNFTIS | |
| | | | VTTEILPVSMTKTSVDCTMYICGDSTECSNLLLQYGSFCTQLNRALTGIAVEQDKNTQEVFAQVKQIYK | |
| | | | TPPIKDFGGFNFSQILPDPSKPSKRSFIEDLLFNKVTLADAGFIKQYGDCLGDIAARDLICAQKFNGLTVL | |
| | | | PPLLTDEMIAQYTSALLAGTITSGWTFGAGAALQIPFAMQMAYRFNGIGVTQNVLYENQKLIANQFNSA | |
| | | | IGKIQDSLSSTASALGKLQDVVNQNAQALNTLVKQLSSNFGAISSVLNDILSRLDPPEAEVQIDRLITGRL | |
| | | | QSLQTYVTQQLIRAAEIRASANLAATKMSECVLGQSKRVDFCGKGYHLMSFPQSAPHGVVFLHVTYVP | |
| | | | AQEKNFTTAPAICHDGKAHFPREGVFVSNGTHWFVTQRNFYEPQIITTDNTFVSGNCDVVIGIVNNTVY | |
| | | | DPLQSELDSIKEELDKIHKNGSGGSGDIIKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP686 | (RR)-mosaic-SpFN | RhGB01-MW719567 RBD & RBD | NVTQLCPFSDVFNRTMFPSVYAWERMSIRDCVADYAVLYNSSISFSTFECYGVSPTKLNDLCFSSVYA | 323 |
| | | from Omicron BA.4/5_ | DYFWVRGDDVKQIAPGKTGVIADYNYKLPDDFVGCVLAWNSRHVDNKQGFYYRLFRHGKIKPYQRDM | |
| | | mosaicSpFN with SARS-1 | SRELYSPSGTPCTVVGFNCYDPLAHYAFASPGIGIGYQPFRVVVLSFQLLNAPATVCGPGSGGSGNIT | |
| | | replacing the original RBD | NLCPFDEVFNATRFASVYAWNRKRISNCVADYSVLYNFAPFFAFKCYGVSPTKLNDLCFTNVYADSFVI | |
| | | | RGNEVSQIAPGQTGNIADYNYKLPDDFTGCVIAWNSNKLDSKVGGNYNYRYRLFRKSNLKPFERDIST | |
| | | | EIYQAGNKPCNGVAGVNCYFPLQSYGFRPTYGVGHQPYRVVVLSFELLHAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATKFPSVYAWERKKIS | |
| | | | NCVADYSVLYNSTFFSTFKCYGVSATKLNDLCFSNVYADSFVVKGDDVRQIAPGQTGVIADYNYKLPD | |
| | | | DFMGCVLAWNTRNIDATSTGNYNYKYRYLRHGKLRPFERDISNVPFSPDGKPCTPPALNCYWPLNDY | |
| | | | GFYTTTGIGYQPYRVVVLSFELLNAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQDVNCTEVPVAIHADQLTPT | |
| | | | WRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAV | |
| | | | EQDKNTQEVFAQVKQIYKTPPIKDFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNQNAQALNTLVKQLSSNFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP687 | (RR)-mosaic-SpFN | bioinformatically derived | NITNLCPFDEVFNATRFPSVYAWERKKISNCVADYSVLYNSTSFSTFKCYGVSPSKLNDLCFTSVYADS | 324 |
| | | Cluster1_RBD (SARS- | FVIRGDEVRQIAPGQTGVIADYNYKLPDDFTGCVIAWNTANQDSGEGGNPNYYYRSSRKSKLKPFER | |
| | | related) &RBD from Omicron | DLSSDEYNVGSQPCNRMGPNCYRTLSTYDFYPTVGVGYQPYRVVVLSFELLNAPATVCGPGSGGSG | |
| | | BA.4/5_mosaicSpFN with | NITNLCPFDEVFNATRFASVYAWNRKRISNCVADYSVLYNFAPFFAFKCYGVSPTKLNDLCFTNVYADS | |
| | | SARS- | FVIRGNEVSQIAPGQTGNIADYNYKLPDDFTGCVIAWNSNKLDSKVGGNYNYRYRLFRKSNLKPFERDI | |
| | | replacing the original RBD | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP688 | (RR)-mosaic-SpFN | bioinformatically derived | NLPDCNIEEWLTAKSVPSPLNWERKTFSNCNFNLSSLLRFVQADSFSCNNIDASKVYGMCFGSISVDK | 325 |
| | | Cluster2_RBD (OC43- | FAIPNSRQVDLQLGNSGFLQSFNYKIDTTATSCQLYYSLPANNVTINNYNPSSWNRRYGFNDDTVFKP | |
| | | related) & | RPAGVLTNHDVVYSQQCFKVPNTFCPCKNNGSCVSSGKGTTNGSGTCPAGTNYRTCENTTWCACS | |
| | | RBD from Omicron BA.4/5_ | CTPDPITASGPRKCPQKKSLVGVGEHCSGLGVDSDYCGNVSCLCSTDAFLGWSADSCLVNDRCNIFA | |
| | | mosaicSpFN with SARS-1 | NIILNGINSGTTCSTGSGGSGNITNLCPFDEVFNATRFASVYAWNRKRISNCVADYSVLYNFAPFFAFK | |
| | | replacing the original RBD | CYGVSPTKLNDLCFTNVYADSFVIRGNEVSQIAPGQTGNIADYNYKLPDDFTGCVIAWNSNKLDSKVG | |
| | | | GNYNYRYRLFRKSNLKPFERDISTEIYQAGNKPCNGVAGVNCYFPLQSYGFRPTYGVGHQPYRVVVL | |
| | | | SFELLHAPATVCGPGSGGSGSSQCVNLTTRTQLPPAYTNSFTRGVYYPDKVFRSSVLHSTQDLFLPFF | |
| | | | SNVTWFHAIHVSGTNGTKRFDNPVLPFNDGVYFASTEKSNIIRGWIFGTTLDSKTQSLLIVNNATNVVIK | |
| | | | VCEFQFCNDPFLGVYYHKNNKSWMESEFRVYSSANNCTFEYVSQPFLMDLEGKQGNFKNLREFVFK | |
| | | | NIDGYFKIYSKHTPINLVRDLPQGFSALEPLVDLPIGINITRFQTLLALHRSYLTPGDSSSGWTAGAAAYY | |
| | | | VGYLQPRTFLLKYNENGTITDAVDCALDPLSETKCTLKSFTVEKGIYQTSNFRVQPTESIVRFPNITNLC | |
| | | | PFGEVFNATKFPSVYAWERKKISNCVADYSVLYNSTFFSTFKCYGVSATKLNDLCFSNVYADSFVVKG | |
| | | | DDVRQIAPGQTGVIADYNYKLPDDFMGCVLAWNTRNIDATSTGNYNYKYRYLRHGKLRPFERDISNVP | |
| | | | FSPDGKPCTPPALNCYWPLNDYGFYTTTGIGYQPYRVVVLSFELLNAPATVCGPKKSTNLVKNKCVNF | |
| | | | NFNGLTGTGVLTESNKKFLPFQQFGRDIADTTDAVRDPQTLEILDITPCSFGGVSVITPGTNTSNQVAV | |
| | | | LYQDVNCTEVPVAIHADQLTPTWRVYSTGSNVFQTRAGCLIGAEHVNNSYECDIPIGAGICASYQTQT | |
| | | | NSPGSASSVASQSIIAYTMSLGAENSVAYSNNSIAIPTNFTISVTTEILPVSMTKTSVDCTMYICGDSTEC | |
| | | | SNLLLQYGSFCTQLNRALTGIAVEQDKNTQEVFAQVKQIYKTPPIKDFGGFNFSQILPDPSKPSKRSFIE | |
| | | | DLLFNKVTLADAGFIKQYGDCLGDIAARDLICAQKFNGLTVLPPLLTDEMIAQYTSALLAGTITSGWTFG | |
| | | | AGAALQIPFAMQMAYRFNGIGVTQNVLYENQKLIANQFNSAIGKIQDSLSSTASALGKLQDVVNQNAQ | |
| | | | ALNTLVKQLSSNFGAISSVLNDILSRLDPPEAEVQIDRLITGRLQSLQTYVTQQLIRAAEIRASANLAATK | |
| | | | MSECVLGQSKRVDFCGKGYHLMSFPQSAPHGVVFLHVTYVPAQEKNFTTAPAICHDGKAHFPREGVF | |
| | | | VSNGTHWFVTQRNFYEPQIITTDNTFVSGNCDVVIGIVNNTVYDPLQSELDSIKEELDKIHKNGSGGSG | |
| | | | DIIKLLNEQVNKEMQSSNLYMSMSSWCYTHSLDGAGLFLFDHAAEEYEHAKKLIIFLNENNVPVQLTSIS | |
| | | | APEHKFEGLTQIFQKAYEHEQHISESINNIVDHAIKSKDHATFNFLQWYVAEQHEEEVLFKDILDKIELIG | |
| | | | NENHGLYLADQYVKGIAKSRKSGS | |
| pCoVP689 | (RR)-mosaic-SpFN | bioinformatically derived | QGVECDFSKLFKAAAPQIYNFSRLVFTNCNYNLTKLLSLFQVDEFSCHQISPSALASGCYSSLTVDYFA | 326 |
| | | Cluster3_RBD (MERS- | YPLSMKSYLQIGSAGEISQFNYKQDFSNPTCRILASVPANVSITKPDKYIYISQCYSLSAYSDVDVPHYV | |
| | | related) & | NPGQYTSIPCLYLTSSGFDEDGQYFRYNLTQFEGQNYLVATGVTVSMTDNLQMVFVITVQYGTDTNS | |
| | | RBD from Omicron BA.4/5_ | VCPGSGGSGNITNLCPFDEVFNATRFASVYAWNRKRISNCVADYSVLYNFAPFFAFKCYGVSPTKLND | |
| | | mosaicSpFN with SARS-1 | LCFTNVYADSFVIRGNEVSQIAPGQTGNIADYNYKLPDDFTGCVIAWNSNKLDSKVGGNYNYRYRLFR | |
| | | replacing the original RBD | KSNLKPFERDISTEIYQAGNKPCNGVAGVNCYFPLQSYGFRPTYGVGHQPYRVVVLSFELLHAPATVC | |
| | | | GPGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATKF | |
| | | | PSVYAWERKKISNCVADYSVLYNSTFFSTFKCYGVSATKLNDLCFSNVYADSFVVKGDDVRQIAPGQT | |
| | | | GVIADYNYKLPDDFMGCVLAWNTRNIDATSTGNYNYKYRYLRHGKLRPFERDISNVPFSPDGKPCTPP | |
| | | | ALNCYWPLNDYGFYTTTGIGYQPYRVVVLSFELLNAPATVCGPKKSTNLVKNKCVNFNFNGLTGTGVL | |
| | | | TESNKKFLPFQQFGRDIADTTDAVRDPQTLEILDITPCSFGGVSVITPGTNTSNQVAVLYQDVNCTEVP | |
| | | | VAIHADQLTPTWRVYSTGSNVFQTRAGCLIGAEHVNNSYECDIPIGAGICASYQTQTNSPGSASSVAS | |
| | | | QSIIAYTMSLGAENSVAYSNNSIAIPTNFTISVTTEILPVSMTKTSVDCTMYICGDSTECSNLLLQYGSFC | |
| | | | TQLNRALTGIAVEQDKNTQEVFAQVKQIYKTPPIKDFGGFNFSQILPDPSKPSKRSFIEDLLFNKVTLAD | |
| | | | AGFIKQYGDCLGDIAARDLICAQKFNGLTVLPPLLTDEMIAQYTSALLAGTITSGWTFGAGAALQIPFAM | |
| | | | QMAYRFNGIGVTQNVLYENQKLIANQFNSAIGKIQDSLSSTASALGKLQDVVNQNAQALNTLVKQLSS | |
| | | | NFGAISSVLNDILSRLDPPEAEVQIDRLITGRLQSLQTYVTQQLIRAAEIRASANLAATKMSECVLGQSK | |
| | | | RVDFCGKGYHLMSFPQSAPHGVVFLHVTYVPAQEKNFTTAPAICHDGKAHFPREGVFVSNGTHWFV | |
| | | | TQRNFYEPQIITTDNTFVSGNCDVVIGIVNNTVYDPLQSELDSIKEELDKIHKNGSGGSGDIIKLLNEQVN | |
| | | | KEMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoVP690 | (RR)-mosaic-SpFN | MER1 (ErinaceusCoV/2012- | ELTECDLDVLFKNDAPIIANYSRRVFTNCNYNLTKLLSLVQVDEFVCHKTTPEALATGCYSSLTVDWFA | 327 |
| | | 174/GER/2012) & RBD from | LPFSMKSTLAIGSAEAISMFNYNQDYSNPTCRIHAAVTANVSTALNFTANANYAYISRCQGVDGKPILLQ | |
| | | Omicron BA.4/5_mosaicSpFN | PGQMTNIACRSGVLARPSDADYFGYSFQGRNYYLGRKSYKPKTDEGDVQMVYVITPKYDKGPDTVC | |
| | | with SARS-1 replacing the | PLKGSGGSGNITNLCPFDEVFNATRFASVYAWNRKRISNCVADYSVLYNFAPFFAFKCYGVSPTKLND | |
| | | original RBD | LCFTNVYADSFVIRGNEVSQIAPGQTGNIADYNYKLPDDFTGCVIAWNSNKLDSKVGGNYNYRYRLFR | |
| | | | KSNLKPFERDISTEIYQAGNKPCNGVAGVNCYFPLQSYGFRPTYGVGHQPYRVVVLSFELLHAPATVC | |
| | | | GPGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATKF | |
| | | | PSVYAWERKKISNCVADYSVLYNSTFFSTFKCYGVSATKLNDLCFSNVYADSFVVKGDDVRQIAPGQT | |
| | | | GVIADYNYKLPDDFMGCVLAWNTRNIDATSTGNYNYKYRYLRHGKLRPFERDISNVPFSPDGKPCTPP | |
| | | | ALNCYWPLNDYGFYTTTGIGYQPYRVVVLSFELLNAPATVCGPKKSTNLVKNKCVNFNFNGLTGTGVL | |
| | | | TESNKKFLPFQQFGRDIADTTDAVRDPQTLEILDITPCSFGGVSVITPGTNTSNQVAVLYQDVNCTEVP | |
| | | | VAIHADQLTPTWRVYSTGSNVFQTRAGCLIGAEHVNNSYECDIPIGAGICASYQTQTNSPGSASSVAS | |
| | | | QSIIAYTMSLGAENSVAYSNNSIAIPTNFTISVTTEILPVSMTKTSVDCTMYICGDSTECSNLLLQYGSFC | |
| | | | TQLNRALTGIAVEQDKNTQEVFAQVKQIYKTPPIKDFGGFNFSQILPDPSKPSKRSFIEDLLFNKVTLAD | |
| | | | AGFIKQYGDCLGDIAARDLICAQKFNGLTVLPPLLTDEMIAQYTSALLAGTITSGWTFGAGAALQIPFAM | |
| | | | QMAYRFNGIGVTQNVLYENQKLIANQFNSAIGKIQDSLSSTASALGKLQDVVNQNAQALNTLVKQLSS | |
| | | | NFGAISSVLNDILSRLDPPEAEVQIDRLITGRLQSLQTYVTQQLIRAAEIRASANLAATKMSECVLGQSK | |
| | | | RVDFCGKGYHLMSFPQSAPHGVVFLHVTYVPAQEKNFTTAPAICHDGKAHFPREGVFVSNGTHWFV | |
| | | | TQRNFYEPQIITTDNTFVSGNCDVVIGIVNNTVYDPLQSELDSIKEELDKIHKNGSGGSGDIIKLLNEQVN | |
| | | | KEMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoVP691 | (RR)-mosaic-SpFN | MER2 (Neoromicia/5038) & | VYPDCNFTELFRERAPTIMQYKREVFTRQNYNLSLLLSLVQVDEFVCDKITPEALATGCYSSLTVDWFA | 328 |
| | | RBD from Omicron BA.4/5_ | FPYAWKSYLAIGSADRIVRFNYNQDYSNPSCRIHSKVNSSIGISYAGAYSYITNCNYGATNKDDVVKPG | |
| | | mosaicSpFN with SARS-1 | GRASQQCITGALNSPTTGQLWAYNFGGVPYRVSRLTYTDHLSDPLDMVYVITVKYEPGAETVCPKQG | |
| | | replacing the original RBD | SGGSGNITNLCPFDEVFNATRFASVYAWNRKRISNCVADYSVLYNFAPFFAFKCYGVSPTKLNDLCFT | |
| | | | NVYADSFVIRGNEVSQIAPGQTGNIADYNYKLPDDFTGCVIAWNSNKLDSKVGGNYNYRYRLFRKSNL | |
| | | | KPFERDISTEIYQAGNKPCNGVAGVNCYFPLQSYGFRPTYGVGHQPYRVVVLSFELLHAPATVCGPG | |
| | | | SGGSGSSQCVNLTTRTQLPPAYTNSFTRGVYYPDKVFRSSVLHSTQDLFLPFFSNVTWFHAIHVSGT | |
| | | | NGTKRFDNPVLPFNDGVYFASTEKSNIIRGWIFGTTLDSKTQSLLIVNNATNVVIKVCEFQFCNDPFLGV | |
| | | | YYHKNNKSWMESEFRVYSSANNCTFEYVSQPFLMDLEGKQGNFKNLREFVFKNIDGYFKIYSKHTPIN | |
| | | | LVRDLPQGFSALEPLVDLPIGINITRFQTLLALHRSYLTPGDSSSGWTAGAAAYYVGYLQPRTFLLKYN | |
| | | | ENGTITDAVDCALDPLSETKCTLKSFTVEKGIYQTSNFRVQPTESIVRFPNITNLCPFGEVFNATKFPSV | |
| | | | YAWERKKISNCVADYSVLYNSTFFSTFKCYGVSATKLNDLCFSNVYADSFVVKGDDVRQIAPGQTGVI | |
| | | | ADYNYKLPDDFMGCVLAWNTRNIDATSTGNYNYKYRYLRHGKLRPFERDISNVPFSPDGKPCTPPAL | |
| | | | NCYWPLNDYGFYTTTGIGYQPYRVVVLSFELLNAPATVCGPKKSTNLVKNKCVNFNFNGLTGTGVLTE | |
| | | | SNKKFLPFQQFGRDIADTTDAVRDPQTLEILDITPCSFGGVSVITPGTNTSNQVAVLYQDVNCTEVPVAI | |
| | | | HADQLTPTWRVYSTGSNVFQTRAGCLIGAEHVNNSYECDIPIGAGICASYQTQTNSPGSASSVASQSII | |
| | | | AYTMSLGAENSVAYSNNSIAIPTNFTISVTTEILPVSMTKTSVDCTMYICGDSTECSNLLLQYGSFCTQL | |
| | | | NRALTGIAVEQDKNTQEVFAQVKQIYKTPPIKDFGGFNFSQILPDPSKPSKRSFIEDLLFNKVTLADAGFI | |
| | | | KQYGDCLGDIAARDLICAQKFNGLTVLPPLLTDEMIAQYTSALLAGTITSGWTFGAGAALQIPFAMQMA | |
| | | | YRFNGIGVTQNVLYENQKLIANQFNSAIGKIQDSLSSTASALGKLQDVVNQNAQALNTLVKQLSSNFGA | |
| | | | ISSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDVVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP692 | (RR)-mosaic-SpFN | MER3 (HKU4 SM3A) & RBD | QATECDFSPMLTGVAPQVYNFKRLVFSNCNYNLTKLLSLFAVDEFSCNGISPDAIARGCYSTLTVDYFA | 329 |
| | | from Omicron BA.4/5_ | YPLSMKSYIRPGSAGNIPLYNYKQSFANPTCRVMASVPANVTITKPDAYGYISKCSRLTGANQDVETPL | |
| | | mosaicSpFN with SARS-1 | YINPGEYSICRDFSPGGFSEDGQVFKRTLTQFEGGGLLIGVGTRVPMTDNLQMSFIIFVQYGTGTDSV | |
| | | replacing the original RBD | CPMLGSGGSGNITNLCPFDEVFNATRFASVYAWNRKRISNCVADYSVLYNFAPFFAFKCYGVSPTKLN | |
| | | | DLCFTNVYADSFVIRGNEVSQIAPGQTGNIADYNYKLPDDFTGCVIAWNSNKLDSKVGGNYNYRYRLF | |
| | | | RKSNLKPFERDISTEIYQAGNKPCNGVAGVNCYFPLQSYGFRPTYGVGHQPYRVVVLSFELLHAPATV | |
| | | | CGPGSGGSGSSQCVNLTTRTQLPPAYTNSFTRGVYYPDKVFRSSVLHSTQDLFLPFFSNVTWFHAIH | |
| | | | VSGTNGTKRFDNPVLPFNDGVYFASTEKSNIIRGWIFGTTLDSKTQSLLIVNNATNVVIKVCEFQFCND | |
| | | | PFLGVYYHKNNKSWMESEFRVYSSANNCTFEYVSQPFLMDLEGKQGNFKNLREFVFKNIDGYFKIYS | |
| | | | KHTPINLVRDLPQGFSALEPLVDLPIGINITRFQTLLALHRSYLTPGDSSSGWTAGAAAYYVGYLQPRTF | |
| | | | LLKYNENGTITDAVDCALDPLSETKCTLKSFTVEKGIYQTSNFRVQPTESIVRFPNITNLCPFGEVFNAT | |
| | | | KFPSVYAWERKKISNCVADYSVLYNSTFFSTFKCYGVSATKLNDLCFSNVYADSFVVKGDDVRQIAPG | |
| | | | QTGVIADYNYKLPDDFMGCVLAWNTRNIDATSTGNYNYKYRYLRHGKLRPFERDISNVPFSPDGKPCT | |
| | | | PPALNCYWPLNDYGFYTTTGIGYQPYRVVVLSFELLNAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDWVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoVP693 | (RR)-mosaic-SpFN | MER4 (BatCoV-Ita2 206645- | QGVECDFSKLFKAAPPQIYNFSRLVFTNCNYNLTKLLSLFHVSEFSCHQVSPSALASGCYSSLTVDYFA | 330 |
| | | 63) & RBD from Omicron | YPLYLASYLQQGSTGEIAQYNYKQDFSNPTCRILASVPANVSIPKPDKYIWLSQCYSFSAYSGDIPHYV | |
| | | BA.4/5_mosaicSpFN with | QPGQYTPCLYLTSSGFDKSYQTNRDMQNKMAATGVTSSMTDNLQMAFVISVQYGTDTNSVCPMGS | |
| | | SARS-1 replacing the | GGSGNITNLCPFDEVFNATRFASVYAWNRKRISNCVADYSVLYNFAPFFAFKCYGVSPTKLNDLCFTN | |
| | | original RBD | VYADSFVIRGNEVSQIAPGQTGNIADYNYKLPDDFTGCVIAWNSNKLDSKVGGNYNYRYRLFRKSNLK | |
| | | | PFERDISTEIYQAGNKPCNGVAGVNCYFPLQSYGFRPTYGVGHQPYRVVVLSFELLHAPATVCGPGS | |
| | | | GGSGSSQCVNLTTRTQLPPAYTNSFTRGVYYPDKVFRSSVLHSTQDLFLPFFSNVTWFHAIHVSGTN | |
| | | | GTKRFDNPVLPFNDGVYFASTEKSNIIRGWIFGTTLDSKTQSLLIVNNATNVVIKVCEFQFCNDPFLGVY | |
| | | | YHKNNKSWMESEFRVYSSANNCTFEYVSQPFLMDLEGKQGNFKNLREFVFKNIDGYFKIYSKHTPINL | |
| | | | VRDLPQGFSALEPLVDLPIGINITRFQTLLALHRSYLTPGDSSSGWTAGAAAYYVGYLQPRTFLLKYNE | |
| | | | NGTITDAVDCALDPLSETKCTLKSFTVEKGIYQTSNFRVQPTESIVRFPNITNLCPFGEVFNATKFPSVY | |
| | | | AWERKKISNCVADYSVLYNSTFFSTFKCYGVSATKLNDLCFSNVYADSFVVKGDDVRQIAPGQTGVIA | |
| | | | DYNYKLPDDFMGCVLAWNTRNIDATSTGNYNYKYRYLRHGKLRPFERDISNVPFSPDGKPCTPPALN | |
| | | | CYWPLNDYGFYTTTGIGYQPYRVWVLSFELLNAPATVCGPKKSTNLVKNKCVNFNFNGLTGTGVLTES | |
| | | | NKKFLPFQQFGRDIADTTDAVRDPQTLEILDITPCSFGGVSVITPGTNTSNQVAVLYQDVNCTEVPVAIH | |
| | | | ADQLTPTWRVYSTGSNVFQTRAGCLIGAEHVNNSYECDIPIGAGICASYQTQTNSPGSASSVASQSIIA | |
| | | | YTMSLGAENSVAYSNNSIAIPTNFTISVTTEILPVSMTKTSVDCTMYICGDSTECSNLLLQYGSFCTQLN | |
| | | | RALTGIAVEQDKNTQEVFAQVKQIYKTPPIKDFGGFNFSQILPDPSKPSKRSFIEDLLFNKVTLADAGFIK | |
| | | | QYGDCLGDIAARDLICAQKFNGLTVLPPLLTDEMIAQYTSALLAGTITSGWTFGAGAALQIPFAMQMAY | |
| | | | RFNGIGVTQNVLYENQKLIANQFNSAIGKIQDSLSSTASALGKLQDVVNQNAQALNTLVKQLSSNFGAI | |
| | | | SSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDWVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| pCoVP694 | (RR)-mosaic-SpFN | MER5 (BatCoV-Ita1 206645- | QGVECDFSKLFKAAPPQIYNFSRLVFTNCNYNLTKLLSLFHVSEFSCHQVSPSALASGCYSSLTVDYFA | 331 |
| | | 40) & RBD from Omicron | YPLYLASYLQQGSTGEIAQYNYKQDFSNPTCRILASVPANVSIPKPDKYIWLSQCYSFSAYSGDVPHYV | |
| | | BA.4/5_mosaicSpFN with | LPGQYTPCLYLTSSGFDNSYQTNRDFQNKMAATGVISSMTDNLQMAFVISVQYGTDTNSVCPMGSG | |
| | | SARS-1 replacing the | GSGNITNLCPFDEVFNATRFASVYAWNRKRISNCVADYSVLYNFAPFFAFKCYGVSPTKLNDLCFTNV | |
| | | original RBD | YADSFVIRGNEVSQIAPGQTGNIADYNYKLPDDFTGCVIAWNSNKLDSKVGGNYNYRYRLFRKSNLKP | |
| | | | FERDISTEIYQAGNKPCNGVAGVNCYFPLQSYGFRPTYGVGHQPYRVVVLSFELLHAPATVCGPGSG | |
| | | | GSGSSQCVNLTTRTQLPPAYTNSFTRGVYYPDKVFRSSVLHSTQDLFLPFFSNVTWFHAIHVSGTNGT | |
| | | | KRFDNPVLPFNDGVYFASTEKSNIIRGWIFGTTLDSKTQSLLIVNNATNVVIKVCEFQFCNDPFLGVYYH | |
| | | | KNNKSWMESEFRVYSSANNCTFEYVSQPFLMDLEGKQGNFKNLREFVFKNIDGYFKIYSKHTPINLVR | |
| | | | DLPQGFSALEPLVDLPIGINITRFQTLLALHRSYLTPGDSSSGWTAGAAAYYVGYLQPRTFLLKYNENG | |
| | | | TITDAVDCALDPLSETKCTLKSFTVEKGIYQTSNFRVQPTESIVRFPNITNLCPFGEVFNATKFPSVYAW | |
| | | | ERKKISNCVADYSVLYNSTFFSTFKCYGVSATKLNDLCFSNVYADSFVVKGDDVRQIAPGQTGVIADYN | |
| | | | YKLPDDFMGCVLAWNTRNIDATSTGNYNYKYRYLRHGKLRPFERDISNVPFSPDGKPCTPPALNCYW | |
| | | | PLNDYGFYTTTGIGYQPYRVVVLSFELLNAPATVCGPKKSTNLVKNKCVNFNFNGLTGTGVLTESNKK | |
| | | | FLPFQQFGRDIADTTDAVRDPQTLEILDITPCSFGGVSVITPGTNTSNQVAVLYQDVNCTEVPVAIHAD | |
| | | | QLTPTWRVYSTGSNVFQTRAGCLIGAEHVNNSYECDIPIGAGICASYQTQTNSPGSASSVASQSIIAYT | |
| | | | MSLGAENSVAYSNNSIAIPTNFTISVTTEILPVSMTKTSVDCTMYICGDSTECSNLLLQYGSFCTQLNRA | |
| | | | LTGIAVEQDKNTQEVFAQVKQIYKTPPIKDFGGFNFSQILPDPSKPSKRSFIEDLLFNKVTLADAGFIKQY | |
| | | | GDCLGDIAARDLICAQKFNGLTVLPPLLTDEMIAQYTSALLAGTITSGWTFGAGAALQIPFAMQMAYRF | |

| | | | | |
|---|---|---|---|---|
| | | | NGIGVTQNVLYENQKLIANQFNSAIGKIQDSLSSTASALGKLQDVVNQNAQALNTLVKQLSSNFGAISS | |
| | | | VLNDILSRLDPPEAEVQIDRLITGRLQSLQTYVTQQLIRAAEIRASANLAATKMSECVLGQSKRVDFCGK | |
| | | | GYHLMSFPQSAPHGVVFLHVTYVPAQEKNFTTAPAICHDGKAHFPREGVFVSNGTHWFVTQRNFYEP | |
| | | | QIITTDNTFVSGNCDVVIGIVNNTVYDPLQSELDSIKEELDKIHKNGSGGSGDIIKLLNEQVNKEMQSSNL | |
| | | | YMSMSSWCYTHSLDGAGLFLFDHAAEEYEHAKKLIIFLNENNVPVQLTSISAPEHKFEGLTQIFQKAYE | |
| | | | HEQHISESINNIVDHAIKSKDHATFNFLQWYVAEQHEEEVLFKDILDKIELIGNENHGLYLADQYVKGIAK | |
| | | | SRKSGS | |
| pCoVP695 | (RR)-mosaic-SpFN | HKU5 & RBD from Omicron | TTQECDFTPMLTGTPPPIYNFKRLVFTNCNYNLTKLLSLFQVSEFSCHQVSPSSLATGCYSSLTVDYFA | 332 |
| | | BA.4/5_mosaicSpFN with | YSTDMSSYLQPGSAGAIVQFNYKQDFSNPTCRVLATVPQNLTTITKPSNYAYLTECYKTSAYGKNYLY | |
| | | SARS-1 replacing the | NAPGAYTPCLSLASRGFSTKYQSHSDGELTTTGYIYPVTGNLQMAFIISVQYGTDTNSVCPMGSGGSG | |
| | | original RBD | NITNLCPFDEVFNATRFASVYAWNRKRISNCVADYSVLYNFAPFFAFKCYGVSPTKLNDLCFTNVYADS | |
| | | | FVIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATKFPSVYAWERKKI | |
| | | | SNCVADYSVLYNSTFFSTFKCYGVSATKLNDLCFSNVYADSFVVKGDDVRQIAPGQTGVIADYNYKLP | |
| | | | DDFMGCVLAWNTRNIDATSTGNYNYKYRYLRHGKLRPFERDISNVPFSPDGKPCTPPALNCYWPLND | |
| | | | YGFYTTTGIGYQPYRVVVLSFELLNAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoVP696 | SpFN | BA.2 Omicron SpFN | SSQCVNLITRTQSYTNSFTRGVYYPDKVFRSSVLHSTQDLFLPFFSNVTWFHAIHVSGTNGTKRFDNP | 333 |
| | | (1B-06-PL) | VLPFNDGVYFASTEKSNIIRGWIFGTTLDSKTQSLLIVNNATNVVIKVCEFQFCNDPFLDVYYHKNNKS | |
| | | | WMESEFRVYSSANNCTFEYVSQPFLMDLEGKQGNFKNLREFVFKNIDGYFKIYSKHTPINLGRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFDEVFNATRFASVYAWNRKRIS | |
| | | | NCVADYSVLYNFAPFFAFKCYGVSPTKLNDLCFTNVYADSFVIRGNEVSQIAPGQTGNIADYNYKLPDD | |
| | | | FTGCVIAWNSNKLDSKVGGNYNYLYRLFRKSNLKPFERDISTEIYQAGNKPCNGVAGFNCYFPLRSYG | |
| | | | FRPTYGVGHQPYRVVVLSFELLHAPATVCGPKKSTNLVKNKCVNFNFNGLTGTGVLTESNKKFLPFQ | |
| | | | QFGRDIADTTDAVRDPQTLEILDITPCSFGGVSVITPGTNTSNQVAVLYQGVNCTEVPVAIHADQLTPT | |
| | | | WRVYSTGSNVFQTRAGCLIGAEYVNNSYECDIPIGAGICASYQTQTKSHGSASSVASQSIIAYTMSLGA | |
| | | | ENSVAYSNNSIAIPTNFTISVTTEILPVSMTKTSVDCTMYICGDSTECSNLLLQYGSFCTQLKRALTGIAV | |
| | | | EQDKNTQEVFAQVKQIYKTPPIKYFGGFNFSQILPDPSKPSKRSFIEDLLFNKVTLADAGFIKQYGDCLG | |
| | | | DIAARDLICAQKFNGLTVLPPLLTDEMIAQYTSALLAGTITSGWTFGAGAALQIPFAMQMAYRFNGIGVT | |
| | | | QNVLYENQKLIANQFNSAIGKIQDSLSSTASALGKLQDVVNHNAQALNTLVKQLSSKFGAISSVLNDILS | |
| | | | RLDPPEAEVQIDRLITGRLQSLQTYVTQQLIRAAEIRASANLAATKMSECVLGQSKRVDFCGKGYHLMS | |
| | | | FPQSAPHGVVFLHVTYVPAQEKNFTTAPAICHDGKAHFPREGVFVSNGTHWFVTQRNFYEPQIITTDN | |
| | | | TFVSGNCDVVIGIVNNTVYDPLQSELDSIKEELDKIHKNGSGGSGDIIKLLNEQVNKEMQSSNLYMSMS | |
| | | | SWCYTHSLDGAGLFLFDHAAEEYEHAKKLIIFLNENNVPVQLTSISAPEHKFEGLTQIFQKAYEHEQHIS | |
| | | | ESINNIVDHAIKSKDHATFNFLQWYVAEQHEEEVLFKDILDKIELIGNENHGLYLADQYVKGIAKSRKSG | |
| | | | S | |
| pCoVP697 | SpFN | BA.4/5 Omicron SpFN | SSQCVNLITRTQSYTNSFTRGVYYPDKVFRSSVLHSTQDLFLPFFSNVTWFHAISGTNGTKRFDNPVL | 334 |
| | | (1B-06-PL) | PFNDGVYFASTEKSNIIRGWIFGTTLDSKTQSLLIVNNATNWVIKVCEFQFCNDPFLDVYYHKNNKSWM | |
| | | | ESEFRVYSSANNCTFEYVSQPFLMDLEGKQGNFKNLREFVFKNIDGYFKIYSKHTPINLGRDLPQGFS | |
| | | | ALEPLVDLPIGINITRFQTLLALHRSYLTPGDSSSGWTAGAAAYYVGYLQPRTFLLKYNENGTITDAVDC | |
| | | | ALDPLSETKCTLKSFTVEKGIYQTSNFRVQPTESIVRFPNITNLCPFDEVFNATRFASVYAWNRKRISNC | |
| | | | VADYSVLYNFAPFFAFKCYGVSPTKLNDLCFTNVYADSFVIRGNEVSQIAPGQTGNIADYNYKLPDDFT | |
| | | | GCVIAWNSNKLDSKVGGNYNYRYRLFRKSNLKPFERDISTEIYQAGNKPCNGVAGVNCYFPLQSYGF | |
| | | | RPTYGVGHQPYRVVVLSFELLHAPATVCGPKKSTNLVKNKCVNFNFNGLTGTGVLTESNKKFLPFQQ | |
| | | | FGRDIADTTDAVRDPQTLEILDITPCSFGGVSVITPGTNTSNQVAVLYQGVNCTEVPVAIHADQLTPTW | |
| | | | RVYSTGSNVFQTRAGCLIGAEYVNNSYECDIPIGAGICASYQTQTKSHGSASSVASQSIIAYTMSLGAE | |
| | | | NSVAYSNNSIAIPTNFTISVTTEILPVSMTKTSVDCTMYICGDSTECSNLLLQYGSFCTQLKRALTGIAVE | |
| | | | QDKNTQEVFAQVKQIYKTPPIKYFGGFNFSQILPDPSKPSKRSFIEDLLFNKVTLADAGFIKQYGDCLGD | |
| | | | IAARDLICAQKFNGLTVLPPLLTDEMIAQYTSALLAGTITSGWTFGAGAALQIPFAMQMAYRFNGIGVTQ | |
| | | | NVLYENQKLIANQFNSAIGKIQDSLSSTASALGKLQDVVNHNAQALNTLVKQLSSKFGAISSVLNDILSR | |
| | | | LDPPEAEVQIDRLITGRLQSLQTYVTQQLIRAAEIRASANLAATKMSECVLGQSKRVDFCGKGYHLMSF | |
| | | | PQSAPHGVVFLHVTYVPAQEKNFTTAPAICHDGKAHFPREGVFVSNGTHWFVTQRNFYEPQIITTDNT | |
| | | | FVSGNCDVVIGIVNNTVYDPLQSELDSIKEELDKIHKNGSGGSGDIIKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| pCoVP698 | RBD-RBD-Ferritin | His8-3c-RBD from Omicron | HHHHHHHHGPLEVLFQGPNITNLCPFDEVFNATRFASVYAWNRKRISNCVADYSVLYNFAPFFAFKCY | 335 |
| | | BA.4/5-GSGGSG-RBD(131)- | GVSPTKLNDLCFTNVYADSFVIRGNEVSQIAPGQTGNIADYNYKLPDDFTGCVIAWNSNKLDSKVGGN | |
| | | Ferritin | YNYRYRLFRKSNLKPFERDISTEIYQAGNKPCNGVAGVNCYFPLQSYGFRPTYGVGHQPYRVVVLSFE | |
| | | | LLHAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP699 | RBD-RBD-Ferritin | His8-3c-RBD from Omicron | HHHHHHHHGPLEVLFQGPNITNLCPFDEVFNATRFASVYAWNRKRISNCVADYSVLYNFAPFFAFKCY | 336 |
| | | BA.2.12.1-GSGGSG- | GVSPTKLNDLCFTNVYADSFVIRGNEVSQIAPGQTGNIADYNYKLPDDFTGCVIAWNSNKLDSKVGGN | |
| | | RBD(131)-Ferritin | YNYQYRLFRKSNLKPFERDISTEIYQAGNKPCNGVAGFNCYFPLRSYGFRPTYGVGHQPYRVVVLSFE | |
| | | | LLHAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP700 | RBD-RBD-Ferritin | His8-3c-RBD from | HHHHHHHHGPLEVLFQGPNITQLCPFNEVFNITSFPSVYAWERMRITNCVADYSVLYNSSASFSTFQC | 337 |
| | | NC_014470.BM4831.BGR.2008- | YGVSPTKLNDLCFSSVYADYFVVKGDDVRQIAPAQTGVIADYNYKLPDDFTGCVIAWNTNSLDSSNEF | |
| | | GSGGSG-RBD(131)-Ferritin | FYRRFRHGKIKPYGRDLSNVLFNPSGGTCSAEGLNCYKPLASYGFTQSSGIGFQPYRVVVLSFELLNA | |
| | | | PATVCGPGSGGSGNITNLCPFGEVFNATRFASVYAWNRKRISNCVADYSVLYNSASFSTFKCYGVSP | |
| | | | TKLNDLCFTNVYADSFVIRGDEVRQIAPGQTGKIADYNYKLPDDFTGCVIAWNSNNLDSKVGGNYNYL | |
| | | | RRLFRKSNLKPFERDISTEIYQAGSTPCNGVEGFNCYFPLQSYGFQPTNGVGYQPYRVVVLSFENKSA | |
| | | | PATVCGPGSGGGGESQVRQQFSKDIEKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP701 | RBD-RBD-Ferritin | His8-3c-BM48-31 RBD-T498W- | HHHHHHHHGPLEVLFQGPNITQLCPFNEVFNITSFPSVYAWERMRITNCVADYSVLYNSSASFSTFQC | 338 |
| | | GSGGSG-RBD(131)- | YGVSPTKLNDLCFSSVYADYFWVKGDDVRQIAPAQTGVIADYNYKLPDDFTGCVIAWNTNSLDSSNEF | |
| | | Ferritin | FYRRFRHGKIKPYGRDLSNVLFNPSGGTCSAEGLNCYKPLASYGFWQSSGIGFQPYRVVVLSFELLNA | |
| | | | PATVCGPGSGGSGNITNLCPFGEVFNATRFASVYAWNRKRISNCVADYSVLYNSASFSTFKCYGVSP | |
| | | | TKLNDLCFTNVYADSFVIRGDEVRQIAPGQTGKIADYNYKLPDDFTGCVIAWNSNNLDSKVGGNYNYL | |
| | | | RRLFRKSNLKPFERDISTEIYQAGSTPCNGVEGFNCYFPLQSYGFQPTNGVGYQPYRVVVLSFENKSA | |
| | | | PATVCGPGSGGGGESQVRQQFSKDIEKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP702 | RBD-RBD-Ferritin | His8-3c-BM48-31 RBD-A493Y- | HHHHHHHHGPLEVLFQGPNITQLCPFNEVFNITSFPSVYAWERMRITNCVADYSVLYNSSASFSTFQC | 339 |
| | | T498W-GSGGSG- | YGVSPTKLNDLCFSSVYADYFVVKGDDVRQIAPAQTGVIADYNYKLPDDFTGCVIAWNTNSLDSSNEF | |
| | | RBD(131)-Ferritin | FYRRFRHGKIKPYGRDLSNVLFNPSGGTCSAEGLNCYKPLYSYGFWQSSGIGFQPYRVVVLSFELLNA | |
| | | | PATVCGPGSGGSGNITNLCPFGEVFNATRFASVYAWNRKRISNCVADYSVLYNSASFSTFKCYGVSP | |
| | | | TKLNDLCFTNVYADSFVIRGDEVRQIAPGQTGKIADYNYKLPDDFTGCVIAWNSNNLDSKVGGNYNYL | |
| | | | RRLFRKSNLKPFERDISTEIYQAGSTPCNGVEGFNCYFPLQSYGFQPTNGVGYQPYRVVVLSFENKSA | |
| | | | PATVCGPGSGGGGESQVRQQFSKDIEKLLNEQVNKEMQSSNLYMSMSSWCYTHSLDGAGLFLFDH | |
| | | | AAEEYEHAKKLIIFLNENNVPVQLTSISAPEHKFEGLTQIFQKAYEHEQHISESINNIVDHAIKSKDHATFN | |
| | | | FLQWYVAEQHEEEVLFKDILDKIELIGNENHGLYLADQYVKGIAKSRKSGS | |
| pCoVP703 | RBD-RBD-Ferritin | His8-3c-RBD from | HHHHHHHHGPLEVLFQGPNITNLCPFGEVFNATTFPSVYAWERKRISNCVADYSVLYNSTSFSTFKCY | 340 |
| | | KC881005.RsSHC014-GSGGSG- | GVSATKLNDLCFSNVYADSFVVKGDDVRQIAPGQTGVIADYNYKLPDDFLGCVLAWNTNSKDSSTSG | |
| | | RBD(131)-Ferritin | NYNYLYRWVRRSKLNPYERDLSNDIYSPGGQSCSAVGPNCYNPLRPYGFFTTAGVGHQPYRVVVLSF | |
| | | | ELLNAPATVCGPGSGGSGNITNLCPFGEVFNATRFASVYAWNRKRISNCVADYSVLYNSASFSTFKCY | |
| | | | GVSPTKLNDLCFTNVYADSFVIRGDEVRQIAPGQTGKIADYNYKLPDDFTGCVIAWNSNNLDSKVGGN | |
| | | | YNYLRRLFRKSNLKPFERDISTEIYQAGSTPCNGVEGFNCYFPLQSYGFQPTNGVGYQPYRVVVLSFE | |
| | | | NKSAPATVCGPGSGGGGESQVRQQFSKDIEKLLNEQVNKEMQSSNLYMSMSSWCYTHSLDGAGLF | |
| | | | LFDHAAEEYEHAKKLIIFLNENNVPVQLTSISAPEHKFEGLTQIFQKAYEHEQHISESINNIVDHAIKSKDH | |
| | | | ATFNFLQWYVAEQHEEEVLFKDILDKIELIGNENHGLYLADQYVKGIAKSRKSGS | |
| pCoVP704 | RBD-RBD-Ferritin | His8-3c-RBD from | HHHHHHHHGPLEVLFQGPNITNLCPFGEVFNATTFPSVYAWERKRISNCVADYSVLYNSTSFSTFKCY | 341 |
| | | KC881007.Bat.WIV1-GSGGSG- | GVSATKLNDLCFSNVYADSFVVKGDDVRQIAPGQTGVIADYNYKLPDDFTGCVLAWNTRNIDATQTGN | |
| | | RBD(131)-Ferritin | YNYKYRSLRHGKLRPFERDISNVPFSPDGKPCTPPAFNCYWPLNDYGFYITNGIGYQPYRVVVLSFEL | |
| | | | LNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP705 | RBD-RBD-Ferritin | His8-3c-RBD from AVP78042_ | HHHHHHHHGPLEVLFQGPNITNVCPFHKVFNATRFPSVYAWERTKISDCIADYTVFYNSTSFSTFKCY | 342 |
| | | BatSARSlikecoronavirus- | GVSPSKLIDLCFTSVYADTFLIRFSEVRQVAPGQTGVIADYNYKLPDDFTGCVIAWNTAKQDTGHYFYR | |
| | | GSGGSG-RBD(131)-Ferritin | SHRSTKLKPFERDLSSDENGVRTLSTYDFNPNVPLEYQATRVVVLSFELLNAPATVCGPGSGGSGNIT | |
| | | | NLCPFGEVFNATRFASVYAWNRKRISNCVADYSVLYNSASFSTFKCYGVSPTKLNDLCFTNVYADSFV | |
| | | | IRGDEVRQIAPGQTGKIADYNYKLPDDFTGCVIAWNSNNLDSKVGGNYNYLRRLFRKSNLKPFERDIST | |
| | | | EIYQAGSTPCNGVEGFNCYFPLQSYGFQPTNGVGYQPYRVVVLSFENKSAPATVCGPGSGGGGESQ | |
| | | | VRQQFSKDIEKLLNEQVNKEMQSSNLYMSMSSWCYTHSLDGAGLFLFDHAAEEYEHAKKLIIFLNENN | |
| | | | VPVQLTSISAPEHKFEGLTQIFQKAYEHEQHISESINNIVDHAIKSKDHATFNFLQWYVAEQHEEEVLFK | |
| | | | DILDKIELIGNENHGLYLADQYVKGIAKSRKSGS | |
| pCoVP706 | RBD-RBD-Ferritin | His8-3c-RBD from MZ937001_ | HHHHHHHHGPLEVLFQGPNITNLCPFGEVFNATTFASVYAWNRKRISNCVADYSVLYNSTSFSTFKCY | 343 |
| | | BANAL20103_Laos_2020- | GVSPTKLNDLCFTNVYADSFVVRGDEVRQIAPGQTGKIADYNYKLPDDFTGCVIAWNSNNLDSKVGG | |
| | | GSGGSG-RBD(131)-Ferritin | NYNYLYRLFRKSNLKPFERDISTEIYQAGSTPCNGVEGFNCYFPLQSYGFHPTNGVGYQPYRVVVLSF | |
| | | | ELLNAPATVCGPGSGGSGNITNLCPFGEVFNATRFASVYAWNRKRISNCVADYSVLYNSASFSTFKCY | |
| | | | GVSPTKLNDLCFTNVYADSFVIRGDEVRQIAPGQTGKIADYNYKLPDDFTGCVIAWNSNNLDSKVGGN | |
| | | | YNYLRRLFRKSNLKPFERDISTEIYQAGSTPCNGVEGFNCYFPLQSYGFQPTNGVGYQPYRVVVLSFE | |
| | | | NKSAPATVCGPGSGGGGESQVRQQFSKDIEKLLNEQVNKEMQSSNLYMSMSSWCYTHSLDGAGLF | |
| | | | LFDHAAEEYEHAKKLIIFLNENNVPVQLTSISAPEHKFEGLTQIFQKAYEHEQHISESINNIVDHAIKSKDH | |
| | | | ATFNFLQWYVAEQHEEEVLFKDILDKIELIGNENHGLYLADQYVKGIAKSRKSGS | |
| pCoVP707 | RBD-RBD-Ferritin | His8-3c-RBD from | HHHHHHHHGPLEVLFQGPNITNLCPFGEVFNATTFASVYAWNRKRISNCVADYSVLYNSTSFSTFKCY | 344 |
| | | MN996532.2013.Bat.RaTG13- | GVSPTKLNDLCFTNVYADSFVITGDEVRQIAPGQTGKIADYNYKLPDDFTGCVIAWNSKHIDAKEGGNF | |
| | | GSGGSG-RBD(131)-Ferritin | NYLYRLFRKANLKPFERDISTEIYQAGSKPCNGQTGLNCYYPLYRYGFYPTDGVGHQPYRVVVLSFEL | |
| | | | LNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP708 | RBD-RBD-Ferritin | His8-3c-RBD from MZ937004_ | HHHHHHHHGPLEVLFQGPNITNFCPFDKVFNATRFPNVYAWQRTKISDCIADYTVLYNSTSFSTFKCY | 345 |
| | | BANAL20247_Laos_2020- | GVSPSKLIDLCFTSVYADTFLIRFSEVRQIAPGETGVIADYNYKLPDDFTGCVLAWNTAQQDIGSYFYR | |
| | | GSGGSG-RBD(131)-Ferritin | SHRAVKLKPFERDLSSDENGVRTLSTYDFNPNVPLDYQATRVVVLSFELLNAPATVCGPGSGGSGNIT | |
| | | | NLCPFGEVFNATRFASVYAWNRKRISNCVADYSVLYNSASFSTFKCYGVSPTKLNDLCFTNVYADSFV | |
| | | | IRGDEVRQIAPGQTGKIADYNYKLPDDFTGCVIAWNSNNLDSKVGGNYNYLRRLFRKSNLKPFERDIST | |
| | | | EIYQAGSTPCNGVEGFNCYFPLQSYGFQPTNGVGYQPYRVVVLSFENKSAPATVCGPGSGGGGESQ | |
| | | | VRQQFSKDIEKLLNEQVNKEMQSSNLYMSMSSWCYTHSLDGAGLFLFDHAAEEYEHAKKLIIFLNENN | |
| | | | VPVQLTSISAPEHKFEGLTQIFQKAYEHEQHISESINNIVDHAIKSKDHATFNFLQWYVAEQHEEEVLFK | |
| | | | DILDKIELIGNENHGLYLADQYVKGIAKSRKSGS | |
| pCoVP709 | RBD-RBD-Ferritin | His8-3c-BANAL-20-247 RBD- | HHHHHHHHGPLEVLFQGPNITNFCPFDKVFNATRFPNVYAWQRTKISDCIADYTVLYNSTSFSTFKCY | 346 |
| | | N498W-GSGGSG-RBD(131)- | GVSPSKLIDLCFTSVYADTFLIRFSEVRQIAPGETGVIADYNYKLPDDFTGCVLAWNTAQQDIGSYFYR | |
| | | Ferritin | SHRAVKLKPFERDLSSDENGVRTLSTYDFWPNVPLDYQATRVVVLSFELLNAPATVCGPGSGGSGNI | |
| | | | TNLCPFGEVFNATRFASVYAWNRKRISNCVADYSVLYNSASFSTFKCYGVSPTKLNDLCFTNVYADSF | |
| | | | VIRGDEVRQIAPGQTGKIADYNYKLPDDFTGCVIAWNSNNLDSKVGGNYNYLRRLFRKSNLKPFERDIS | |
| | | | TEIYQAGSTPCNGVEGFNCYFPLQSYGFQPTNGVGYQPYRVVVLSFENKSAPATVCGPGSGGGGES | |
| | | | QVRQQFSKDIEKLLNEQVNKEMQSSNLYMSMSSWCYTHSLDGAGLFLFDHAAEEYEHAKKLIIFLNEN | |
| | | | NVPVQLTSISAPEHKFEGLTQIFQKAYEHEQHISESINNIVDHAIKSKDHATFNFLQWYVAEQHEEEVLF | |
| | | | KDILDKIELIGNENHGLYLADQYVKGIAKSRKSGS | |
| pCoVP710 | RBD-RBD-Ferritin | His8-3c-BANAL-20-247 RBD- | HHHHHHHHGPLEVLFQGPNITNFCPFDKVFNATRFPNVYAWQRTKISDCIADYTVLYNSTSFSTFKCY | 347 |
| | | S493Y-N498W-GSGGSG- | GVSPSKLIDLCFTSVYADTFLIRFSEVRQIAPGETGVIADYNYKLPDDFTGCVLAWNTAQQDIGSYFYR | |
| | | RBD(131)-Ferritin | SHRAVKLKPFERDLSSDENGVRTLYTYDFWPNVPLDYQATRVVVLSFELLNAPATVCGPGSGGSGNI | |
| | | | TNLCPFGEVFNATRFASVYAWNRKRISNCVADYSVLYNSASFSTFKCYGVSPTKLNDLCFTNVYADSF | |
| | | | VIRGDEVRQIAPGQTGKIADYNYKLPDDFTGCVIAWNSNNLDSKVGGNYNYLRRLFRKSNLKPFERDIS | |
| | | | TEIYQAGSTPCNGVEGFNCYFPLQSYGFQPTNGVGYQPYRVVVLSFENKSAPATVCGPGSGGGGES | |
| | | | QVRQQFSKDIEKLLNEQVNKEMQSSNLYMSMSSWCYTHSLDGAGLFLFDHAAEEYEHAKKLIIFLNEN | |
| | | | NVPVQLTSISAPEHKFEGLTQIFQKAYEHEQHISESINNIVDHAIKSKDHATFNFLQWYVAEQHEEEVLF | |
| | | | KDILDKIELIGNENHGLYLADQYVKGIAKSRKSGS | |
| pCoVP711 | RBD-RBD-Ferritin | His8-3c-RBD from ATO98145_ | HHHHHHHHGPLEVLFQGPNITNLCPFDAVFNATRFPSVYAWERVKISNCVADYTAFYNSTSFSTFKCY | 348 |
| | | BatSARSlikecoronavirus- | GVSPSKLIDLCFTSVYADTFLIRFSEVRQVAPGETGVIADYNYKLPDDFTGCVIAWNTAKQDVGSYFYR | |
| | | GSGGSG-RBD(131)-Ferritin | SHRSSKLKPFERDLSSDENGVRTLSTYDFNPNVPLDYQATRVVVLSFELLNAPATVCGPGSGGSGNIT | |
| | | | NLCPFGEVFNATRFASVYAWNRKRISNCVADYSVLYNSASFSTFKCYGVSPTKLNDLCFTNVYADSFV | |
| | | | IRGDEVRQIAPGQTGKIADYNYKLPDDFTGCVIAWNSNNLDSKVGGNYNYLRRLFRKSNLKPFERDIST | |
| | | | EIYQAGSTPCNGVEGFNCYFPLQSYGFQPTNGVGYQPYRVVVLSFENKSAPATVCGPGSGGGGESQ | |
| | | | VRQQFSKDIEKLLNEQVNKEMQSSNLYMSMSSWCYTHSLDGAGLFLFDHAAEEYEHAKKLIIFLNENN | |
| | | | VPVQLTSISAPEHKFEGLTQIFQKAYEHEQHISESINNIVDHAIKSKDHATFNFLQWYVAEQHEEEVLFK | |
| | | | DILDKIELIGNENHGLYLADQYVKGIAKSRKSGS | |
| pCoVP712 | RBD-RBD-Ferritin | His8-3c-RBD from JX993987. | HHHHHHHHGPLEVLFQGPNITNRCPFDKVFNATRFPSVYAWERTKISDCVADYTVLYNSTSFSTFKCY | 349 |
| | | 2011.BatRp.Shaanxi2011- | GVSPSKLIDLCFTSVYADTFLIRSSEVRQVAPGETGVIADYNYKLPDDFTGCVIAWNTANQDQGQYYY | |
| | | GSGGSG-RBD(131)-Ferritin | RSSRKEKLKPFERDLSSDENGVYTLSTYDFYPSVPLDYQATRVVVLSFELLNAPATVCGPGSGGSGNI | |
| | | | TNLCPFGEVFNATRFASVYAWNRKRISNCVADYSVLYNSASFSTFKCYGVSPTKLNDLCFTNVYADSF | |
| | | | VIRGDEVRQIAPGQTGKIADYNYKLPDDFTGCVIAWNSNNLDSKVGGNYNYLRRLFRKSNLKPFERDIS | |
| | | | TEIYQAGSTPCNGVEGFNCYFPLQSYGFQPTNGVGYQPYRVVVLSFENKSAPATVCGPGSGGGGES | |
| | | | QVRQQFSKDIEKLLNEQVNKEMQSSNLYMSMSSWCYTHSLDGAGLFLFDHAAEEYEHAKKLIIFLNEN | |
| | | | NVPVQLTSISAPEHKFEGLTQIFQKAYEHEQHISESINNIVDHAIKSKDHATFNFLQWYVAEQHEEEVLF | |
| | | | KDILDKIELIGNENHGLYLADQYVKGIAKSRKSGS | |
| pCoVP713 | RBD-RBD-Ferritin | His8-3c-RBD from KJ473812. | HHHHHHHHGPLEVLFQGPNITNLCPFDKVFNATRFPSVYAWERTKISDCVADYTVFYNSTSFSTENCY | 350 |
| | | BtRfBetaCoV.HeB2013- | GVSPSKLIDLCFTSVYADTFLIRFSEVRQVAPGQTGVIADYNYKLPDDFTGCVIAWNTAKQDVGSYFYR | |
| | | GSGGSG-RBD(131)-Ferritin | SHRSSKLKPFERDLSSEENGVRTLSTYDFNQYVPLEYQATRVVVLSFELLNAPATVCGPGSGGSGNIT | |
| | | | NLCPFGEVFNATRFASVYAWNRKRISNCVADYSVLYNSASFSTFKCYGVSPTKLNDLCFTNVYADSFV | |
| | | | IRGDEVRQIAPGQTGKIADYNYKLPDDFTGCVIAWNSNNLDSKVGGNYNYLRRLFRKSNLKPFERDIST | |
| | | | EIYQAGSTPCNGVEGFNCYFPLQSYGFQPTNGVGYQPYRVVVLSFENKSAPATVCGPGSGGGGESQ | |
| | | | VRQQFSKDIEKLLNEQVNKEMQSSNLYMSMSSWCYTHSLDGAGLFLFDHAAEEYEHAKKLIIFLNENN | |
| | | | VPVQLTSISAPEHKFEGLTQIFQKAYEHEQHISESINNIVDHAIKSKDHATFNFLQWYVAEQHEEEVLFK | |
| | | | DILDKIELIGNENHGLYLADQYVKGIAKSRKSGS | |
| pCoVP714 | RBD-RBD-Ferritin | His8-3c-RBD from DQ071615. | HHHHHHHHGPLEVLFQGPNITNRCPFDKVFNATRFPNVYAWERTKISDCVADYTVLYNSTSFSTFKCY | 351 |
| | | 2003.Bat.Rp3-GSGGSG- | GVSPSKLIDLCFTSVYADTFLIRSSEVRQVAPGETGVIADYNYKLPDDFTGCVIAWNTAKQDQGQYYY | |
| | | RBD(131)-Ferritin | RSHRKTKLKPFERDLSSDENGVRTLSTYDFYPSVPVAYQATRVVVLSFELLNAPATVCGPGSGGSGNI | |
| | | | TNLCPFGEVFNATRFASVYAWNRKRISNCVADYSVLYNSASFSTFKCYGVSPTKLNDLCFTNVYADSF | |
| | | | VIRGDEVRQIAPGQTGKIADYNYKLPDDFTGCVIAWNSNNLDSKVGGNYNYLRRLFRKSNLKPFERDIS | |
| | | | TEIYQAGSTPCNGVEGFNCYFPLQSYGFQPTNGVGYQPYRVVVLSFENKSAPATVCGPGSGGGGES | |
| | | | QVRQQFSKDIEKLLNEQVNKEMQSSNLYMSMSSWCYTHSLDGAGLFLFDHAAEEYEHAKKLIIFLNEN | |
| | | | NVPVQLTSISAPEHKFEGLTQIFQKAYEHEQHISESINNIVDHAIKSKDHATFNFLQWYVAEQHEEEVLF | |
| | | | KDILDKIELIGNENHGLYLADQYVKGIAKSRKSGS | |
| pCoVP715 | RBD-RBD-Ferritin | His8-3c-RBD from ACU31032_ | HHHHHHHHGPLEVLFQGPNITNRCPFDKVFNASRFPNVYAWERTKISDCVADYTVLYNSTSFSTFKCY | 352 |
| | | Rs_672_2006-GSGGSG- | GVSPSKLIDLCFTSVYADTFLIRSSEVRQVAPGETGVIADYNYKLPDDFTGCVIAWNTAKQDQGQYYY | |
| | | RBD(131)-Ferritin | RSSRKTKLKPFERDLTSDENGVRTLSTYDFYPNVPIEYQATRVVVLSFELLNAPATVCGPGSGGSGNIT | |
| | | | NLCPFGEVFNATRFASVYAWNRKRISNCVADYSVLYNSASFSTFKCYGVSPTKLNDLCFTNVYADSFV | |
| | | | IRGDEVRQIAPGQTGKIADYNYKLPDDFTGCVIAWNSNNLDSKVGGNYNYLRRLFRKSNLKPFERDIST | |
| | | | EIYQAGSTPCNGVEGFNCYFPLQSYGFQPTNGVGYQPYRVVVLSFENKSAPATVCGPGSGGGGESQ | |
| | | | VRQQFSKDIEKLLNEQVNKEMQSSNLYMSMSSWCYTHSLDGAGLFLFDHAAEEYEHAKKLIIFLNENN | |
| | | | VPVQLTSISAPEHKFEGLTQIFQKAYEHEQHISESINNIVDHAIKSKDHATFNFLQWYVAEQHEEEVLFK | |
| | | | DILDKIELIGNENHGLYLADQYVKGIAKSRKSGS | |
| pCoVP716 | RBD-RBD-Ferritin | His8-3c-RBD from MZ081380_ | HHHHHHHHGPLEVLFQGPNITNLCPFSQVFNATRFPSVYAWTRERISNCIADYSVLYNSTSFSTFRCY | 353 |
| | | bat_Yunnan_RsYN04_2020- | GVSPTKLNDLCFSNVYADSMVVRGDEVRQIAPSQTGVIADYNYKLPDDFTGCVIAWNSKAKDENGQY | |
| | | GSGGSG-RBD(131)-Ferritin | FYRLFRKSKLLPFQRDVSNVTYGSGKNDGCNPSEADCYWPLLKYGFTSSVSQDYQPYRVVVLSFELL | |
| | | | NAPATVCGPGSGGSGNITNLCPFGEVFNATRFASVYAWNRKRISNCVADYSVLYNSASFSTFKCYGV | |
| | | | SPTKLNDLCFTNVYADSFVIRGDEVRQIAPGQTGKIADYNYKLPDDFTGCVIAWNSNNLDSKVGGNYN | |
| | | | YLRRLFRKSNLKPFERDISTEIYQAGSTPCNGVEGFNCYFPLQSYGFQPTNGVGYQPYRVVVLSFENK | |
| | | | SAPATVCGPGSGGGGESQVRQQFSKDIEKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP717 | RBD-RBD-Ferritin | His8-3c-RsYN04 RBD-T498W- | HHHHHHHHGPLEVLFQGPNITNLCPFSQVFNATRFPSVYAWTRERISNCIADYSVLYNSTSFSTFRCY | 354 |
| | | GSGGSG-RBD(131)- | GVSPTKLNDLCFSNVYADSMVVRGDEVRQIAPSQTGVIADYNYKLPDDFTGCVIAWNSKAKDENGQY | |
| | | Ferritin | FYRLFRKSKLLPFQRDVSNVTYGSGKNDGCNPSEADCYWPLLKYGFWSSVSQDYQPYRVWVLSFELL | |
| | | | NAPATVCGPGSGGSGNITNLCPFGEVFNATRFASVYAWNRKRISNCVADYSVLYNSASFSTFKCYGV | |
| | | | SPTKLNDLCFTNVYADSFVIRGDEVRQIAPGQTGKIADYNYKLPDDFTGCVIAWNSNNLDSKVGGNYN | |
| | | | YLRRLFRKSNLKPFERDISTEIYQAGSTPCNGVEGFNCYFPLQSYGFQPTNGVGYQPYRVVVLSFENK | |
| | | | SAPATVCGPGSGGGGESQVRQQFSKDIEKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP718 | RBD-RBD-Ferritin | His8-3c-RsYN04 RBD-L493Y- | HHHHHHHHGPLEVLFQGPNITNLCPFSQVFNATRFPSVYAWTRERISNCIADYSVLYNSTSFSTFRCY | 355 |
| | | T498W-GSGGSG- | GVSPTKLNDLCFSNVYADSMVVRGDEVRQIAPSQTGVIADYNYKLPDDFTGCVIAWNSKAKDENGQY | |
| | | RBD(131)-Ferritin | FYRLFRKSKLLPFQRDVSNVTYGSGKNDGCNPSEADCYWPLYKYGFWSSVSQDYQPYRVVVLSFEL | |
| | | | LNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP719 | RBD-RBD-Ferritin | His8-3c-Khosta2-MZ190138 | HHHHHHHHGPLEVLFQGPNMTNICPFDQVFNKTQFPSVYAWERVRISDCVSDYTVLYNSSASFSTFK | 356 |
| | | RBD-GSGGSG-RBD(131)- | CYGVSPTKLNDLCFSGVYADYFVVKGDHVHQIAPGQTGVIADYNYKLPSEFVGCILAWNTRTIDSKRG | |
| | | Ferritin | FYYRLFRHGNIRPYERDTSNVPYNAAGGTCNQPGTHNCYEPLQDYGFTSTSGVGYQPFRVVVLSFEL | |
| | | | LNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP720 | RBD-RBD-Ferritin | His8-3c-Khosta-2 RBD- | HHHHHHHHGPLEVLFQGPNMTNICPFDQVFNKTQFPSVYAWERVRISDCVSDYTVLYNSSASFSTFK | 357 |
| | | T498W-GSGGSG-RBD(131)- | CYGVSPTKLNDLCFSGVYADYFVVKGDHVHQIAPGQTGVIADYNYKLPSEFVGCILAWNTRTIDSKRG | |
| | | Ferritin | FYYRLFRHGNIRPYERDTSNVPYNAAGGTCNQPGTHNCYEPLQDYGFWSTSGVGYQPFRVVVLSFE | |
| | | | LLNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP721 | RBD-RBD-Ferritin | His8-3c-Khosta-2 RBD- | HHHHHHHHGPLEVLFQGPNMTNICPFDQVFNKTQFPSVYAWERVRISDCVSDYTVLYNSSASFSTFK | 358 |
| | | Q493Y-T498W-GSGGSG- | CYGVSPTKLNDLCFSGVYADYFVVKGDHVHQIAPGQTGVIADYNYKLPSEFVGCILAWNTRTIDSKRG | |
| | | RBD(131)-Ferritin | FYYRLFRHGNIRPYERDTSNVPYNAAGGTCNQPGTHNCYEPLYDYGFWSTSGVGYQPFRVVVLSFEL | |
| | | | LNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP722 | RBD-RBD-Ferritin | His8-3c-KY352407_BtKY72_ | HHHHHHHHGPLEVLFQGPNITNLCPFGQVFNASNFPSVYAWERLRISDCVADYAVLYNSSSSFSTFKC | 359 |
| | | bat_Kenya RBD-GSGGSG- | YGVSPTKLNDLCFSSVYADYFWVKGDDVRQIAPAQTGVIADYNYKLPDDFTGCVLAWNTNSVDSKSG | |
| | | RBD(131)-Ferritin | NNFYYRLFRHGKIKPYERDISNVLYNSAGGTCSSISQLGCYEPLKSYGFTPTVGVGYQPYRVVVLSFEL | |
| | | | LNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP723 | RBD-RBD-Ferritin | His8-3c-BtKY72 RBD-T498W- | HHHHHHHHGPLEVLFQGPNITNLCPFGQVFNASNFPSVYAWERLRISDCVADYAVLYNSSSSFSTFKC | 360 |
| | | GSGGSG-RBD(131)- | YGVSPTKLNDLCFSSVYADYFVVKGDDVRQIAPAQTGVIADYNYKLPDDFTGCVLAWNTNSVDSKSG | |
| | | Ferritin | NNFYYRLFRHGKIKPYERDISNVLYNSAGGTCSSISQLGCYEPLKSYGFWPTVGVGYQPYRVVVLSFE | |
| | | | LLNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP724 | RBD-RBD-Ferritin | His8-3c-BtKY72 RBD-K493Y- | HHHHHHHHGPLEVLFQGPNITNLCPFGQVFNASNFPSVYAWERLRISDCVADYAVLYNSSSSFSTFKC | 361 |
| | | T498W-GSGGSG- | YGVSPTKLNDLCFSSVYADYFWVKGDDVRQIAPAQTGVIADYNYKLPDDFTGCVLAWNTNSVDSKSG | |
| | | RBD(131)-Ferritin | NNFYYRLFRHGKIKPYERDISNVLYNSAGGTCSSISQLGCYEPLYSYGFWPTVGVGYQPYRVVVLSFE | |
| | | | LLNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP725 | RBD-RBD-Ferritin | His8-3c-RaTG15_Ra7909 RBD- | HHHIHHHGPLEVLFQGPNITNLCPFSQVFNATRFPSVYAWTRERISNCIADYSVLYNSTSFSTFRCY | 362 |
| | | GSGGSG-RBD(131)- | GVSPTKLNDLCFSNVYADSMVVRGDEVRQIAPSQTGVIADYNYKLPDDFTGCVIAWNSKAKDENGQY | |
| | | Ferritin | FYRLFRKSKLLPFQRDVSNVTYGSGKNDGCNPSEADCYWPLLKYGFTGSVSQDYQPYRVVVLSFELL | |
| | | | NAPATVCGPGSGGSGNITNLCPFGEVFNATRFASVYAWNRKRISNCVADYSVLYNSASFSTFKCYGV | |
| | | | SPTKLNDLCFTNVYADSFVIRGDEVRQIAPGQTGKIADYNYKLPDDFTGCVIAWNSNNLDSKVGGNYN | |
| | | | YLRRLFRKSNLKPFERDISTEIYQAGSTPCNGVEGFNCYFPLQSYGFQPTNGVGYQPYRVVVLSFENK | |
| | | | SAPATVCGPGSGGGGESQVRQQFSKDIEKLLNEQVNKEMQSSNLYMSMSSWCYTHSLDGAGLFLFD | |
| | | | HAAEEYEHAKKLIIFLNENNVPVQLTSISAPEHKFEGLTQIFQKAYEHEQHISESINNIVDHAIKSKDHATF | |
| | | | NFLQWYVAEQHEEEVLFKDILDKIELIGNENHGLYLADQYVKGIAKSRKSGS | |
| pCoVP726 | RBD-RBD-Ferritin | His8-3c-HKU3-1_ | HHHHHHHHGPLEVLFQGPNITNRCPFDKVFNATRFPNVYAWERTKISDCVADYTVLYNSTSFSTFKCY | 363 |
| | | DQ022305.2005 RBD-GSGGSG- | GVSPSKLIDLCFTSVYADTFLIRSSEVRQVAPGETGVIADYNYKLPDDFTGCVIAWNTAKHDTGNYYYR | |
| | | RBD(131)-Ferritin | SHRKTKLKPFERDLSSDDGNGVYTLSTYDFNPNVPVAYQATRVVVLSFELLNAPATVCGPGSGGSGNI | |
| | | | TNLCPFGEVFNATRFASVYAWNRKRISNCVADYSVLYNSASFSTFKCYGVSPTKLNDLCFTNVYADSF | |
| | | | VIRGDEVRQIAPGQTGKIADYNYKLPDDFTGCVIAWNSNNLDSKVGGNYNYLRRLFRKSNLKPFERDIS | |
| | | | TEIYQAGSTPCNGVEGFNCYFPLQSYGFQPTNGVGYQPYRVVVLSFENKSAPATVCGPGSGGGGES | |
| | | | QVRQQFSKDIEKLLNEQVNKEMQSSNLYMSMSSWCYTHSLDGAGLFLFDHAAEEYEHAKKLIIFLNEN | |
| | | | NVPVQLTSISAPEHKFEGLTQIFQKAYEHEQHISESINNIVDHAIKSKDHATFNFLQWYVAEQHEEEVLF | |
| | | | KDILDKIELIGNENHGLYLADQYVKGIAKSRKSGS | |
| pCoVP727 | RBD-RBD-Ferritin | His8-3c-RhGB01-MW719567 | HHHHHHHHGPLEVLFQGPNVTQLCPFSDVFNRTMFPSVYAWERMSIRDCVADYAVLYNSSISFSTFE | 364 |
| | | RBD-GSGGSG-RBD(131)- | CYGVSPTKLNDLCFSSVYADYFVVRGDDVKQIAPGKTGVIADYNYKLPDDFVGCVLAWNSRHVDNKQ | |
| | | Ferritin | GFYYRLFRHGKIKPYQRDMSRELYSPSGTPCTVVGFNCYDPLAHYAFASPGIGIGYQPFRVVVLSFQL | |
| | | | LNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP728 | RBD-RBD-Ferritin | His8-3c-bioinformatically | HHHHHHHHGPLEVLFQGPNITNLCPFDEVFNATRFPSVYAWERKKISNCVADYSVLYNSTSFSTFKCY | 365 |
| | | derived Cluster1_RBD (SARS- | GVSPSKLNDLCFTSVYADSFVIRGDEVRQIAPGQTGVIADYNYKLPDDFTGCVIAWNTANQDSGEGGN | |
| | | related)-GSGGSG-RBD(131)- | PNYYYRSSRKSKLKPFERDLSSDEYNVGSQPCNRMGPNCYRTLSTYDFYPTVGVGYQPYRVVVLSFE | |
| | | Ferritin | LLNAPATVCGPGSGGSGNITNLCPFGEVFNATRFASVYAWNRKRISNCVADYSVLYNSASFSTFKCYG | |
| | | | VSPTKLNDLCFTNVYADSFVIRGDEVRQIAPGQTGKIADYNYKLPDDFTGCVIAWNSNNLDSKVGGNY | |
| | | | NYLRRLFRKSNLKPFERDISTEIYQAGSTPCNGVEGFNCYFPLQSYGFQPTNGVGYQPYRVVVLSFEN | |
| | | | KSAPATVCGPGSGGGGESQVRQQFSKDIEKLLNEQVNKEMQSSNLYMSMSSWCYTHSLDGAGLFLF | |
| | | | DHAAEEYEHAKKLIIFLNENNVPVQLTSISAPEHKFEGLTQIFQKAYEHEQHISESINNIVDHAIKSKDHAT | |
| | | | FNFLQWYVAEQHEEEVLFKDILDKIELIGNENHGLYLADQYVKGIAKSRKSGS | |
| pCoVP729 | RBD-RBD-Ferritin | His8-3c-bioinformatically | HHHHHHHHGPLEVLFQGPNLPDCNIEEWLTAKSVPSPLNWERKTFSNCNFNLSSLLRFVQADSFSCN | 366 |
| | | derived Cluster2_RBD (OC43- | NIDASKVYGMCFGSISVDKFAIPNSRQVDLQLGNSGFLQSFNYKIDTTATSCQLYYSLPANNVTINNYN | |
| | | related)-GSGGSG-RBD(131)- | PSSWNRRYGFNDDTVFKPRPAGVLTNHDVVYSQQCFKVPNTFCPCKNNGSCVSSGKGTTNGSGTC | |
| | | Ferritin | PAGTNYRTCENTTWCACSCTPDPITASGPRKCPQKKSLVGVGEHCSGLGVDSDYCGNVSCLCSTDA | |
| | | | FLGWSADSCLVNDRCNIFANIILNGINSGTTCSTGSGGSGNITNLCPFGEVFNATRFASVYAWNRKRIS | |
| | | | NCVADYSVLYNSASFSTFKCYGVSPTKLNDLCFTNVYADSFVIRGDEVRQIAPGQTGKIADYNYKLPDD | |
| | | | FTGCVIAWNSNNLDSKVGGNYNYLRRLFRKSNLKPFERDISTEIYQAGSTPCNGVEGFNCYFPLQSYG | |
| | | | FQPTNGVGYQPYRVVVLSFENKSAPATVCGPGSGGGGESQVRQQFSKDIEKLLNEQVNKEMQSSNL | |
| | | | YMSMSSWCYTHSLDGAGLFLFDHAAEEYEHAKKLIIFLNENNVPVQLTSISAPEHKFEGLTQIFQKAYE | |
| | | | HEQHISESINNIVDHAIKSKDHATFNFLQWYVAEQHEEEVLFKDILDKIELIGNENHGLYLADQYVKGIAK | |
| | | | SRKSGS | |
| pCoVP730 | RBD-RBD-Ferritin | His8-3c-bioinformatically | HHHHHHHHGPLEVLFQGPQGVECDFSKLFKAAAPQIYNFSRLVFTNCNYNLTKLLSLFQVDEFSCHQI | 367 |
| | | derived Cluster3_RBD (MERS- | SPSALASGCYSSLTVDYFAYPLSMKSYLQIGSAGEISQFNYKQDFSNPTCRILASVPANVSITKPDKYIYI | |
| | | related)-GSGGSG-RBD(131)- | SQCYSLSAYSDVDVPHYVNPGQYTSIPCLYLTSSGFDEDGQYFRYNLTQFEGQNYLVATGVTVSMTD | |
| | | Ferritin | NLQMVFVITVQYGTDTNSVCPGSGGSGNITNLCPFGEVFNATRFASVYAWNRKRISNCVADYSVLYN | |
| | | | SASFSTFKCYGVSPTKLNDLCFTNVYADSFVIRGDEVRQIAPGQTGKIADYNYKLPDDFTGCVIAWNS | |
| | | | NNLDSKVGGNYNYLRRLFRKSNLKPFERDISTEIYQAGSTPCNGVEGFNCYFPLQSYGFQPTNGVGY | |
| | | | QPYRVVVLSFENKSAPATVCGPGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pCoVP731 | RBD-RBD-Ferritin | His8-3c-MER1 (ErinaceusCoV/ | HHHHHHHHGPLEVLFQGPELTECDLDVLFKNDAPIIANYSRRVFTNCNYNLTKLLSLVQVDEFVCHKTT | 368 |
| | | 2012-174/GER/2012) RBD- | PEALATGCYSSLTVDWFALPFSMKSTLAIGSAEAISMFNYNQDYSNPTCRIHAAVTANVSTALNFTANA | |
| | | GSGGSG-RBD(131)-Ferritin | NYAYISRCQGVDGKPILLQPGQMTNIACRSGVLARPSDADYFGYSFQGRNYYLGRKSYKPKTDEGDV | |
| | | | QMVYVITPKYDKGPDTVCPLKGSGGSGNITNLCPFGEVFNATRFASVYAWNRKRISNCVADYSVLYNS | |
| | | | ASFSTFKCYGVSPTKLNDLCFTNVYADSFVIRGDEVRQIAPGQTGKIADYNYKLPDDFTGCVIAWNSN | |
| | | | NLDSKVGGNYNYLRRLFRKSNLKPFERDISTEIYQAGSTPCNGVEGFNCYFPLQSYGFQPTNGVGYQ | |
| | | | PYRVVVLSFENKSAPATVCGPGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoVP732 | RBD-RBD-Ferritin | His8-3c-MER2 (Neoromicia/ | HHHHHHHHGPLEVLFQGPVYPDCNFTELFRERAPTIMQYKREVFTRCNYNLSLLLSLVQVDEFVCDKI | 369 |
| | | 5038) RBD-GSGGSG- | TPEALATGCYSSLTVDWFAFPYAWKSYLAIGSADRIVRFNYNQDYSNPSCRIHSKVNSSIGISYAGAYS | |
| | | RBD(131)-Ferritin | YITNCNYGATNKDDVVKPGGRASQQCITGALNSPTTGQLWAYNFGGVPYRVSRLTYTDHLSDPLDMV | |
| | | | YVITVKYEPGAETVCPKQGSGGSGNITNLCPFGEVFNATRFASVYAWNRKRISNCVADYSVLYNSASF | |
| | | | STFKCYGVSPTKLNDLCFTNVYADSFVIRGDEVRQIAPGQTGKIADYNYKLPDDFTGCVIAWNSNNLD | |
| | | | SKVGGNYNYLRRLFRKSNLKPFERDISTEIYQAGSTPCNGVEGFNCYFPLQSYGFQPTNGVGYQPYR | |
| | | | VVVLSFENKSAPATVCGPGSGGGGESQVRQQFSKDIEKLLNEQVNKEMQSSNLYMSMSSWCYTHSL | |
| | | | DGAGLFLFDHAAEEYEHAKKLIIFLNENNVPVQLTSISAPEHKFEGLTQIFQKAYEHEQHISESINNIVDH | |
| | | | AIKSKDHATFNFLQWYVAEQHEEEVLFKDILDKIELIGNENHGLYLADQYVKGIAKSRKSGS | |
| pCoVP733 | RBD-RBD-Ferritin | His8-3c-MER3 (HKU4 SM3A) | HHHHHHHHGPLEVLFQGPQATECDFSPMLTGVAPQVYNFKRLVFSNCNYNLTKLLSLFAVDEFSCNGI | 370 |
| | | RBD-GSGGSG-RBD(131)- | SPDAIARGCYSTLTVDYFAYPLSMKSYIRPGSAGNIPLYNYKQSFANPTCRVMASVPANVTITKPDAYG | |
| | | Ferritin | YISKCSRLTGANQDVETPLYINPGEYSICRDFSPGGFSEDGQVFKRTLTQFEGGGLLIGVGTRVPMTD | |
| | | | NLQMSFIIFVQYGTGTDSVCPMLGSGGSGNITNLCPFGEVFNATRFASVYAWNRKRISNCVADYSVLY | |
| | | | NSASFSTFKCYGVSPTKLNDLCFTNVYADSFVIRGDEVRQIAPGQTGKIADYNYKLPDDFTGCVIAWN | |
| | | | SNNLDSKVGGNYNYLRRLFRKSNLKPFERDISTEIYQAGSTPCNGVEGFNCYFPLQSYGFQPTNGVG | |
| | | | YQPYRVVVLSFENKSAPATVCGPGSGGGGESQVRQQFSKDIEKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESI | |
| | | | NNIVDHAIKSKDHATFNFLQWYVAEQHEEEVLFKDILDKIELIGNENHGLYLADQYVKGIAKSRKSGS | |
| pCoVP734 | RBD-RBD-Ferritin | His8-3c-MER4 (BatCoV-Ita2 | HHHHHHHHGPLEVLFQGPQGVECDFSKLFKAAPPQIYNFSRLVFTNCNYNLTKLLSLFHVSEFSCHQV | 371 |
| | | 206645-63) RBD-GSGGSG- | SPSALASGCYSSLTVDYFAYPLYLASYLQQGSTGEIAQYNYKQDFSNPTCRILASVPANVSIPKPDKYI | |
| | | RBD(131)-Ferritin | WLSQCYSFSAYSGDIPHYVQPGQYTPCLYLTSSGFDKSYQTNRDMQNKMAATGVTSSMTDNLQMAF | |
| | | | VISVQYGTDTNSVCPMGSGGSGNITNLCPFGEVFNATRFASVYAWNRKRISNCVADYSVLYNSASFS | |
| | | | TFKCYGVSPTKLNDLCFTNVYADSFVIRGDEVRQIAPGQTGKIADYNYKLPDDFTGCVIAWNSNNLDS | |
| | | | KVGGNYNYLRRLFRKSNLKPFERDISTEIYQAGSTPCNGVEGFNCYFPLQSYGFQPTNGVGYQPYRV | |
| | | | WVLSFENKSAPATVCGPGSGGGGESQVRQQFSKDIEKLLNEQVNKEMQSSNLYMSMSSWCYTHSLD | |
| | | | GAGLFLFDHAAEEYEHAKKLIIFLNENNVPVQLTSISAPEHKFEGLTQIFQKAYEHEQHISESINNIVDHAI | |
| | | | KSKDHATFNFLQWYVAEQHEEEVLFKDILDKIELIGNENHGLYLADQYVKGIAKSRKSGS | |
| pCoVP735 | RBD-RBD-Ferritin | His8-3c-MER5 (BatCoV-Ita1 | HHHHHHHHGPLEVLFQGPQGVECDFSKLFKAAPPQIYNFSRLVFTNCNYNLTKLLSLFHVSEFSCHQV | 372 |
| | | 206645-40) RBD-GSGGSG- | SPSALASGCYSSLTVDYFAYPLYLASYLQQGSTGEIAQYNYKQDFSNPTCRILASVPANVSIPKPDKYI | |
| | | RBD(131)-Ferritin | WLSQCYSFSAYSGDVPHYVLPGQYTPCLYLTSSGFDNSYQTNRDFQNKMAATGVISSMTDNLQMAF | |
| | | | VISVQYGTDTNSVCPMGSGGSGNITNLCPFGEVFNATRFASVYAWNRKRISNCVADYSVLYNSASFS | |
| | | | TFKCYGVSPTKLNDLCFTNVYADSFVIRGDEVRQIAPGQTGKIADYNYKLPDDFTGCVIAWNSNNLDS | |
| | | | KVGGNYNYLRRLFRKSNLKPFERDISTEIYQAGSTPCNGVEGFNCYFPLQSYGFQPTNGVGYQPYRV | |
| | | | WLSFENKSAPATVCGPGSGGGGESQVRQQFSKDIEKLLNEQVNKEMQSSNLYMSMSSWCYTHSLD | |
| | | | GAGLFLFDHAAEEYEHAKKLIIFLNENNVPVQLTSISAPEHKFEGLTQIFQKAYEHEQHISESINNIVDHAI | |
| | | | KSKDHATFNFLQWYVAEQHEEEVLFKDILDKIELIGNENHGLYLADQYVKGIAKSRKSGS | |
| pCoVP736 | RBD-RBD-Ferritin | His8-3c-HKU5 RBD-GSGGSG- | HHHHHHHHGPLEVLFQGPTTQECDFTPMLTGTPPPIYNFKRLVFTNCNYNLTKLLSLFQVSEFSCHQV | 373 |
| | | RBD(131)-Ferritin | SPSSLATGCYSSLTVDYFAYSTDMSSYLQPGSAGAIVQFNYKQDFSNPTCRVLATVPQNLTTITKPSN | |
| | | | YAYLTECYKTSAYGKNYLYNAPGAYTPCLSLASRGFSTKYQSHSDGELTTTGYIYPVTGNLQMAFIISV | |
| | | | QYGTDTNSVCPMGSGGSGNITNLCPFGEVFNATRFASVYAWNRKRISNCVADYSVLYNSASFSTFKC | |
| | | | YGVSPTKLNDLCFTNVYADSFVIRGDEVRQIAPGQTGKIADYNYKLPDDFTGCVIAWNSNNLDSKVGG | |
| | | | NYNYLRRLFRKSNLKPFERDISTEIYQAGSTPCNGVEGFNCYFPLQSYGFQPTNGVGYQPYRVVVLSF | |
| | | | ENKSAPATVCGPGSGGGGESQVRQQFSKDIEKLLNEQVNKEMQSSNLYMSMSSWCYTHSLDGAGL | |
| | | | FLFDHAAEEYEHAKKLIIFLNENNVPVQLTSISAPEHKFEGLTQIFQKAYEHEQHISESINNIVDHAIKSKD | |
| | | | HATFNFLQWYVAEQHEEEVLFKDILDKIELIGNENHGLYLADQYVKGIAKSRKSGS | |
| pCoVP737 | R-S1-Ferritin | His8-3c-RBD from Omicron | HHHHHHHHGPLEVLFQGPNITNLCPFDEVFNATRFASVYAWNRKRISNCVADYSVLYNFAPFFAFKCY | 374 |
| | | BA.4/5-GSGGSG-S1-Ferritin | GVSPTKLNDLCFTNVYADSFVIRGNEVSQIAPGQTGNIADYNYKLPDDFTGCVIAWNSNKLDSKVGGN | |
| | | | YNYRYRLFRKSNLKPFERDISTEIYQAGNKPCNGVAGVNCYFPLQSYGFRPTYGVGHQPYRVVVLSFE | |
| | | | LLHAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP738 | R-S1-Ferritin | His8-3c-RBD from Omicron | HHHHHHHHGPLEVLFQGPNITNLCPFDEVFNATRFASVYAWNRKRISNCVADYSVLYNFAPFFAFKCY | 375 |
| | | BA.2.12.1-GSGGSG-S1- | GVSPTKLNDLCFTNVYADSFVIRGNEVSQIAPGQTGNIADYNYKLPDDFTGCVIAWNSNKLDSKVGGN | |
| | | Ferritin | YNYQYRLFRKSNLKPFERDISTEIYQAGNKPCNGVAGFNCYFPLRSYGFRPTYGVGHQPYRVVVLSFE | |
| | | | LLHAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP739 | R-S1-Ferritin | His8-3c-RBD from | HHHHHHHHGPLEVLFQGPNITQLCPFNEVFNITSFPSVYAWERMRITNCVADYSVLYNSSASFSTFQC | 376 |
| | | NC_014470.BM4831.BGR.2008- | YGVSPTKLNDLCFSSVYADYFWVKGDDVRQIAPAQTGVIADYNYKLPDDFTGCVIAWNTNSLDSSNEF | |
| | | GSGGSG-S1-Ferritin | FYRRFRHGKIKPYGRDLSNVLFNPSGGTCSAEGLNCYKPLASYGFTQSSGIGFQPYRVVVLSFELLNA | |
| | | | PATVCGPGSGGSGSSQCVNLTTRTQLPPAYTNSFTRGVYYPDKVFRSSVLHSTQDLFLPFFSNVTWF | |
| | | | HAIHVSGTNGTKRFDNPVLPFNDGVYFASTEKSNIIRGWIFGTTLDSKTQSLLIVNNATNVVIKVCEFQF | |
| | | | CNDPFLGVYYHKNNKSWMESEFRVYSSANNCTFEYVSQPFLMDLEGKQGNFKNLREFVFKNIDGYFK | |
| | | | IYSKHTPINLVRDLPQGFSALEPLVDLPIGINITRFQTLLALHRSYLTPGDSSSGWTAGAAAYYVGYLQP | |
| | | | RTFLLKYNENGTITDAVDCALDPLSETKCTLKSFTVEKGIYQTSNFRVQPTESIVRFPNITNLCPFGEVF | |
| | | | NATRFASVYAWNRKRISNCVADYSVLYNSASFSTFKCYGVSPTKLNDLCFTNVYADSFVIRGDEVRQI | |
| | | | APGQTGKIADYNYKLPDDFTGCVIAWNSNNLDSKVGGNYNYLYRLFRKSNLKPFERDISTEIYQAGST | |
| | | | PCNGVEGFNCYFPLQSYGFQPTNGVGYQPYRVVVLSFELLHAPATVCGPKKSTNLVKNKCVNFNFNG | |
| | | | LTGTGVLTESNKKFLPFQQFGRDIADTTDAVRDPQTLEILDITPCSFGGVSVITPGTNTSNQVAVLYQD | |
| | | | VNCTEVPVAIHADQLTPTWRVYSTGSNVFQTRAGCLIGAEHVNNSYECDIPIGAGICASYQTGGSQSII | |
| | | | AYTGSGGGGESQVRQQFSKDIEKLLNEQVNKEMQSSNLYMSMSSWCYTHSLDGAGLFLFDHAAEEY | |
| | | | EHAKKLIIFLNENNVPVQLTSISAPEHKFEGLTQIFQKAYEHEQHISESINNIVDHAIKSKDHATFNFLQW | |
| | | | YVAEQHEEEVLFKDILDKIELIGNENHGLYLADQYVKGIAKSRKSGS | |
| pCoVP740 | R-S1-Ferritin | His8-3c-BM48-31 RBD-T498W- | HHHHHHHHGPLEVLFQGPNITQLCPFNEVFNITSFPSVYAWERMRITNCVADYSVLYNSSASFSTFQC | 377 |
| | | GSGGSG-S1-Ferritin | YGVSPTKLNDLCFSSVYADYFWVKGDDVRQIAPAQTGVIADYNYKLPDDFTGCVIAWNTNSLDSSNEF | |
| | | | FYRRFRHGKIKPYGRDLSNVLFNPSGGTCSAEGLNCYKPLASYGFWQSSGIGFQPYRVVVLSFELLNA | |
| | | | PATVCGPGSGGSGSSQCVNLTTRTQLPPAYTNSFTRGVYYPDKVFRSSVLHSTQDLFLPFFSNVTWF | |
| | | | HAIHVSGTNGTKRFDNPVLPFNDGVYFASTEKSNIIRGWIFGTTLDSKTQSLLIVNNATNVVIKVCEFQF | |
| | | | CNDPFLGVYYHKNNKSWMESEFRVYSSANNCTFEYVSQPFLMDLEGKQGNFKNLREFVFKNIDGYFK | |
| | | | IYSKHTPINLVRDLPQGFSALEPLVDLPIGINITRFQTLLALHRSYLTPGDSSSGWTAGAAAYYVGYLQP | |
| | | | RTFLLKYNENGTITDAVDCALDPLSETKCTLKSFTVEKGIYQTSNFRVQPTESIVRFPNITNLCPFGEVF | |
| | | | NATRFASVYAWNRKRISNCVADYSVLYNSASFSTFKCYGVSPTKLNDLCFTNVYADSFVIRGDEVRQI | |
| | | | APGQTGKIADYNYKLPDDFTGCVIAWNSNNLDSKVGGNYNYLYRLFRKSNLKPFERDISTEIYQAGST | |
| | | | PCNGVEGFNCYFPLQSYGFQPTNGVGYQPYRVVVLSFELLHAPATVCGPKKSTNLVKNKCVNFNFNG | |
| | | | LTGTGVLTESNKKFLPFQQFGRDIADTTDAVRDPQTLEILDITPCSFGGVSVITPGTNTSNQVAVLYQD | |
| | | | VNCTEVPVAIHADQLTPTWRVYSTGSNVFQTRAGCLIGAEHVNNSYECDIPIGAGICASYQTGGSQSII | |
| | | | AYTGSGGGGESQVRQQFSKDIEKLLNEQVNKEMQSSNLYMSMSSWCYTHSLDGAGLFLFDHAAEEY | |
| | | | EHAKKLIIFLNENNVPVQLTSISAPEHKFEGLTQIFQKAYEHEQHISESINNIVDHAIKSKDHATFNFLQW | |
| | | | YVAEQHEEEVLFKDILDKIELIGNENHGLYLADQYVKGIAKSRKSGS | |
| pCoVP741 | R-S1-Ferritin | His8-3c-BM48-31 RBD-A493Y- | HHHHHHHHGPLEVLFQGPNITQLCPFNEVFNITSFPSVYAWERMRITNCVADYSVLYNSSASFSTFQC | 378 |
| | | T498W-GSGGSG-S1- | YGVSPTKLNDLCFSSVYADYFVVKGDDVRQIAPAQTGVIADYNYKLPDDFTGCVIAWNTNSLDSSNEF | |
| | | Ferritin | FYRRFRHGKIKPYGRDLSNVLFNPSGGTCSAEGLNCYKPLYSYGFWQSSGIGFQPYRVVVLSFELLNA | |
| | | | PATVCGPGSGGSGSSQCVNLTTRTQLPPAYTNSFTRGVYYPDKVFRSSVLHSTQDLFLPFFSNVTWF | |
| | | | HAIHVSGTNGTKRFDNPVLPFNDGVYFASTEKSNIIRGWIFGTTLDSKTQSLLIVNNATNVVIKVCEFQF | |
| | | | CNDPFLGVYYHKNNKSWMESEFRVYSSANNCTFEYVSQPFLMDLEGKQGNFKNLREFVFKNIDGYFK | |
| | | | IYSKHTPINLVRDLPQGFSALEPLVDLPIGINITRFQTLLALHRSYLTPGDSSSGWTAGAAAYYVGYLQP | |
| | | | RTFLLKYNENGTITDAVDCALDPLSETKCTLKSFTVEKGIYQTSNFRVQPTESIVRFPNITNLCPFGEVF | |
| | | | NATRFASVYAWNRKRISNCVADYSVLYNSASFSTFKCYGVSPTKLNDLCFTNVYADSFVIRGDEVRQI | |
| | | | APGQTGKIADYNYKLPDDFTGCVIAWNSNNLDSKVGGNYNYLYRLFRKSNLKPFERDISTEIYQAGST | |
| | | | PCNGVEGFNCYFPLQSYGFQPTNGVGYQPYRVVVLSFELLHAPATVCGPKKSTNLVKNKCVNFNFNG | |
| | | | LTGTGVLTESNKKFLPFQQFGRDIADTTDAVRDPQTLEILDITPCSFGGVSVITPGTNTSNQVAVLYQD | |
| | | | VNCTEVPVAIHADQLTPTWRVYSTGSNVFQTRAGCLIGAEHVNNSYECDIPIGAGICASYQTGGSQSII | |
| | | | AYTGSGGGGESQVRQQFSKDIEKLLNEQVNKEMQSSNLYMSMSSWCYTHSLDGAGLFLFDHAAEEY | |
| | | | EHAKKLIIFLNENNVPVQLTSISAPEHKFEGLTQIFQKAYEHEQHISESINNIVDHAIKSKDHATFNFLQW | |
| | | | YVAEQHEEEVLFKDILDKIELIGNENHGLYLADQYVKGIAKSRKSGS | |
| pCoVP742 | R-S1-Ferritin | His8-3c-RBD from KC881005. | HHHHHHHHGPLEVLFQGPNITNLCPFGEVFNATTFPSVYAWERKRISNCVADYSVLYNSTSFSTFKCY | 379 |
| | | RsSHC014-GSGGSG-S1- | GVSATKLNDLCFSNVYADSFVVKGDDVRQIAPGQTGVIADYNYKLPDDFLGCVLAWNTNSKDSSTSG | |
| | | Ferritin | NYNYLYRWVRRSKLNPYERDLSNDIYSPGGQSCSAVGPNCYNPLRPYGFFTTAGVGHQPYRVVVLSF | |
| | | | ELLNAPATVCGPGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLKSFTVEKGIYQTSNFRVQPTESIVRFPNITNLCP | |
| | | | FGEVFNATRFASVYAWNRKRISNCVADYSVLYNSASFSTFKCYGVSPTKLNDLCFTNVYADSFVIRGD | |
| | | | EVRQIAPGQTGKIADYNYKLPDDFTGCVIAWNSNNLDSKVGGNYNYLYRLFRKSNLKPFERDISTEIYQ | |
| | | | AGSTPCNGVEGFNCYFPLQSYGFQPTNGVGYQPYRVWVLSFELLHAPATVCGPKKSTNLVKNKCVNF | |
| | | | NFNGLTGTGVLTESNKKFLPFQQFGRDIADTTDAVRDPQTLEILDITPCSFGGVSVITPGTNTSNQVAV | |
| | | | LYQDVNCTEVPVAIHADQLTPTWRVYSTGSNVFQTRAGCLIGAEHVNNSYECDIPIGAGICASYQTGG | |
| | | | SQSIIAYTGSGGGGESQVRQQFSKDIEKLLNEQVNKEMQSSNLYMSMSSWCYTHSLDGAGLFLFDHA | |
| | | | AEEYEHAKKLIIFLNENNVPVQLTSISAPEHKFEGLTQIFQKAYEHEQHISESINNIVDHAIKSKDHATFNF | |
| | | | LQWYVAEQHEEEVLFKDILDKIELIGNENHGLYLADQYVKGIAKSRKSGS | |
| pCoVP743 | R-S1-Ferritin | His8-3c-RBD from KC881007. | HHHHHHHHGPLEVLFQGPNITNLCPFGEVENATTFPSVYAWERKRISNCVADYSVLYNSTSFSTFKCY | 380 |
| | | Bat.WIV1-GSGGSG-S1- | GVSATKLNDLCFSNVYADSFVVKGDDVRQIAPGQTGVIADYNYKLPDDFTGCVLAWNTRNIDATQTGN | |
| | | Ferritin | YNYKYRSLRHGKLRPFERDISNVPFSPDGKPCTPPAFNCYWPLNDYGFYITNGIGYQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP744 | R-S1-Ferritin | His8-3c-RBD from AVP78042_ | HHHHHHHHGPLEVLFQGPNITNVCPFHKVFNATRFPSVYAWERTKISDCIADYTVFYNSTSFSTFKCY | 381 |
| | | BatSARSlikecoronavirus- | GVSPSKLIDLCFTSVYADTFLIRFSEVRQVAPGQTGVIADYNYKLPDDFTGCVIAWNTAKQDTGHYFYR | |
| | | GSGGSG-S1-Ferritin | SHRSTKLKPFERDLSSDENGVRTLSTYDFNPNVPLEYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP745 | R-S1-Ferritin | His8-3c-RBD from MZ937001_ | HHHHHHHHGPLEVLFQGPNITNLCPFGEVFNATTFASVYAWNRKRISNCVADYSVLYNSTSFSTFKCY | 382 |
| | | BANAL20103_Laos_2020- | GVSPTKLNDLCFTNVYADSFVVRGDEVRQIAPGQTGKIADYNYKLPDDFTGCVIAWNSNNLDSKVGG | |
| | | GSGGSG-S1-Ferritin | NYNYLYRLFRKSNLKPFERDISTEIYQAGSTPCNGVEGFNCYFPLQSYGFHPTNGVGYQPYRVVVLSF | |
| | | | ELLNAPATVCGPGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLKSFTVEKGIYQTSNFRVQPTESIVRFPNITNLCP | |
| | | | FGEVFNATRFASVYAWNRKRISNCVADYSVLYNSASFSTFKCYGVSPTKLNDLCFTNVYADSFVIRGD | |
| | | | EVRQIAPGQTGKIADYNYKLPDDFTGCVIAWNSNNLDSKVGGNYNYLYRLFRKSNLKPFERDISTEIYQ | |
| | | | AGSTPCNGVEGFNCYFPLQSYGFQPTNGVGYQPYRVVVLSFELLHAPATVCGPKKSTNLVKNKCVNF | |
| | | | NFNGLTGTGVLTESNKKFLPFQQFGRDIADTTDAVRDPQTLEILDITPCSFGGVSVITPGTNTSNQVAV | |
| | | | LYQDVNCTEVPVAIHADQLTPTWRVYSTGSNVFQTRAGCLIGAEHVNNSYECDIPIGAGICASYQTGG | |
| | | | SQSIIAYTGSGGGGESQVRQQFSKDIEKLLNEQVNKEMQSSNLYMSMSSWCYTHSLDGAGLFLFDHA | |
| | | | AEEYEHAKKLIIFLNENNVPVQLTSISAPEHKFEGLTQIFQKAYEHEQHISESINNIVDHAIKSKDHATFNF | |
| | | | LQWYVAEQHEEEVLFKDILDKIELIGNENHGLYLADQYVKGIAKSRKSGS | |
| pCoVP746 | R-S1-Ferritin | His8-3c-RBD from | HHHHHHHHGPLEVLFQGPNITNLCPFGEVFNATTFASVYAWNRKRISNCVADYSVLYNSTSFSTFKCY | 383 |
| | | MN996532.2013.Bat.RaTG13- | GVSPTKLNDLCFTNVYADSFVITGDEVRQIAPGQTGKIADYNYKLPDDFTGCVIAWNSKHIDAKEGGNF | |
| | | GSGGSG-S1-Ferritin | NYLYRLFRKANLKPFERDISTEIYQAGSKPCNGQTGLNCYYPLYRYGFYPTDGVGHQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP747 | R-S1-Ferritin | His8-3c-RBD from MZ937004_ | HHHHHHHHGPLEVLFQGPNITNFCPFDKVFNATRFPNVYAWQRTKISDCIADYTVLYNSTSFSTFKCY | 384 |
| | | BANAL20247_Laos_2020- | GVSPSKLIDLCFTSVYADTFLIRFSEVRQIAPGETGVIADYNYKLPDDFTGCVLAWNTAQQDIGSYFYR | |
| | | GSGGSG-S1-Ferritin | SHRAVKLKPFERDLSSDENGVRTLSTYDFNPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVWVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP748 | R-S1-Ferritin | His8-3c-BANAL-20-247 RBD- | HHHHHHHHGPLEVLFQGPNITNFCPFDKVFNATRFPNVYAWQRTKISDCIADYTVLYNSTSFSTFKCY | 385 |
| | | N498W-GSGGSG-S1- | GVSPSKLIDLCFTSVYADTFLIRFSEVRQIAPGETGVIADYNYKLPDDFTGCVLAWNTAQQDIGSYFYR | |
| | | Ferritin | SHRAVKLKPFERDLSSDENGVRTLSTYDFWPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP749 | R-S1-Ferritin | His8-3c-BANAL-20-247 RBD- | HHHHHHHHGPLEVLFQGPNITNFCPFDKVFNATRFPNVYAWQRTKISDCIADYTVLYNSTSFSTFKCY | 386 |
| | | S493Y-N498W-GSGGSG- | GVSPSKLIDLCFTSVYADTFLIRFSEVRQIAPGETGVIADYNYKLPDDFTGCVLAWNTAQQDIGSYFYR | |
| | | S1-Ferritin | SHRAVKLKPFERDLSSDENGVRTLYTYDFWPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP750 | R-S1-Ferritin | His8-3c-RBD from ATO98145_ | HHHHHHHHGPLEVLFQGPNITNLCPFDAVFNATRFPSVYAWERVKISNCVADYTAFYNSTSFSTFKCY | 387 |
| | | BatSARSlikecoronavirus- | GVSPSKLIDLCFTSVYADTFLIRFSEVRQVAPGETGVIADYNYKLPDDFTGCVIAWNTAKQDVGSYFYR | |
| | | GSGGSG-S1-Ferritin | SHRSSKLKPFERDLSSDENGVRTLSTYDFNPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP751 | R-S1-Ferritin | His8-3c-RBD from JX993987. | HHHHHHHHGPLEVLFQGPNITNRCPFDKVFNATRFPSVYAWERTKISDCVADYTVLYNSTSFSTFKCY | 388 |
| | | 2011.BatRp.Shaanxi2011- | GVSPSKLIDLCFTSVYADTFLIRSSEVRQVAPGETGVIADYNYKLPDDFTGCVIAWNTANQDQGQYYY | |
| | | GSGGSG-S1-Ferritin | RSSRKEKLKPFERDLSSDENGVYTLSTYDFYPSVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP752 | R-S1-Ferritin | His8-3c-RBD from KJ473812. | HHHHHHHHGPLEVLFQGPNITNLCPFDKVFNATRFPSVYAWERTKISDCVADYTVFYNSTSFSTENCY | 389 |
| | | BtRfBetaCoV.HeB2013- | GVSPSKLIDLCFTSVYADTFLIRFSEVRQVAPGQTGVIADYNYKLPDDFTGCVIAWNTAKQDVGSYFYR | |
| | | GSGGSG-S1-Ferritin | SHRSSKLKPFERDLSSEENGVRTLSTYDFNQYVPLEYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP753 | R-S1-Ferritin | His8-3c-RBD from DQ071615. | HHHHHHHHGPLEVLFQGPNITNRCPFDKVFNATRFPNVYAWERTKISDCVADYTVLYNSTSFSTFKCY | 390 |
| | | 2003.Bat.Rp3-GSGGSG-S1- | GVSPSKLIDLCFTSVYADTFLIRSSEVRQVAPGETGVIADYNYKLPDDFTGCVIAWNTAKQDQGQYYY | |
| | | Ferritin | RSHRKTKLKPFERDLSSDENGVRTLSTYDFYPSVPVAYQATRVVVLSFELLNAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGENCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP754 | R-S1-Ferritin | His8-3c-RBD from ACU31032_ | HHHHHHHHGPLEVLFQGPNITNRCPFDKVFNASRFPNVYAWERTKISDCVADYTVLYNSTSFSTFKCY | 391 |
| | | Rs_672_2006-GSGGSG- | GVSPSKLIDLCFTSVYADTFLIRSSEVRQVAPGETGVIADYNYKLPDDFTGCVIAWNTAKQDQGQYYY | |
| | | S1-Ferritin | RSSRKTKLKPFERDLTSDENGVRTLSTYDFYPNVPIEYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP755 | R-S1-Ferritin | His8-3c-RBD from MZ081380_ | HHHHHHHHGPLEVLFQGPNITNLCPFSQVFNATRFPSVYAWTRERISNCIADYSVLYNSTSFSTFRCY | 392 |
| | | bat_Yunnan_RsYN04_2020- | GVSPTKLNDLCFSNVYADSMWVRGDEVRQIAPSQTGVIADYNYKLPDDFTGCVIAWNSKAKDENGQY | |
| | | GSGGSG-S1-Ferritin | FYRLFRKSKLLPFQRDVSNVTYGSGKNDGCNPSEADCYWPLLKYGFTSSVSQDYQPYRVVVLSFELL | |
| | | | NAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLKSFTVEKGIYQTSNFRVQPTESIVRFPNITNLCPFG | |
| | | | EVFNATRFASVYAWNRKRISNCVADYSVLYNSASFSTFKCYGVSPTKLNDLCFTNVYADSFVIRGDEV | |
| | | | RQIAPGQTGKIADYNYKLPDDFTGCVIAWNSNNLDSKVGGNYNYLYRLFRKSNLKPFERDISTEIYQAG | |
| | | | STPCNGVEGFNCYFPLQSYGFQPTNGVGYQPYRVVVLSFELLHAPATVCGPKKSTNLVKNKCVNFNF | |
| | | | NGLTGTGVLTESNKKFLPFQQFGRDIADTTDAVRDPQTLEILDITPCSFGGVSVITPGTNTSNQVAVLY | |
| | | | QDVNCTEVPVAIHADQLTPTWRVYSTGSNVFQTRAGCLIGAEHVNNSYECDIPIGAGICASYQTGGSQ | |
| | | | SIIAYTGSGGGGESQVRQQFSKDIEKLLNEQVNKEMQSSNLYMSMSSWCYTHSLDGAGLFLFDHAAE | |
| | | | EYEHAKKLIIFLNENNVPVQLTSISAPEHKFEGLTQIFQKAYEHEQHISESINNIVDHAIKSKDHATFNFLQ | |
| | | | WYVAEQHEEEVLFKDILDKIELIGNENHGLYLADQYVKGIAKSRKSGS | |
| pCoVP756 | R-S1-Ferritin | His8-3c-RsYN04 RBD-T498W- | HHHHHHHHGPLEVLFQGPNITNLCPFSQVFNATRFPSVYAWTRERISNCIADYSVLYNSTSFSTFRCY | 393 |
| | | GSGGSG-S1-Ferritin | GVSPTKLNDLCFSNVYADSMVVRGDEVRQIAPSQTGVIADYNYKLPDDFTGCVIAWNSKAKDENGQY | |
| | | | FYRLFRKSKLLPFQRDVSNVTYGSGKNDGCNPSEADCYWPLLKYGFWSSVSQDYQPYRVVVLSFELL | |
| | | | NAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLKSFTVEKGIYQTSNFRVQPTESIVRFPNITNLCPFG | |
| | | | EVFNATRFASVYAWNRKRISNCVADYSVLYNSASFSTFKCYGVSPTKLNDLCFTNVYADSFVIRGDEV | |
| | | | RQIAPGQTGKIADYNYKLPDDFTGCVIAWNSNNLDSKVGGNYNYLYRLFRKSNLKPFERDISTEIYQAG | |
| | | | STPCNGVEGFNCYFPLQSYGFQPTNGVGYQPYRVVVLSFELLHAPATVCGPKKSTNLVKNKCVNFNF | |
| | | | NGLTGTGVLTESNKKFLPFQQFGRDIADTTDAVRDPQTLEILDITPCSFGGVSVITPGTNTSNQVAVLY | |
| | | | QDVNCTEVPVAIHADQLTPTWRVYSTGSNVFQTRAGCLIGAEHVNNSYECDIPIGAGICASYQTGGSQ | |
| | | | SIIAYTGSGGGGESQVRQQFSKDIEKLLNEQVNKEMQSSNLYMSMSSWCYTHSLDGAGLFLFDHAAE | |
| | | | EYEHAKKLIIFLNENNVPVQLTSISAPEHKFEGLTQIFQKAYEHEQHISESINNIVDHAIKSKDHATFNFLQ | |
| | | | WYVAEQHEEEVLFKDILDKIELIGNENHGLYLADQYVKGIAKSRKSGS | |
| pCoVP757 | R-S1-Ferritin | His8-3c-RsYN04 RBD-L493Y- | HHHHHHHHGPLEVLFQGPNITNLCPFSQVFNATRFPSVYAWTRERISNCIADYSVLYNSTSFSTFRCY | 394 |
| | | T498W-GSGGSG-S1- | GVSPTKLNDLCFSNVYADSMVVRGDEVRQIAPSQTGVIADYNYKLPDDFTGCVIAWNSKAKDENGQY | |
| | | Ferritin | FYRLFRKSKLLPFQRDVSNVTYGSGKNDGCNPSEADCYWPLYKYGFWSSVSQDYQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP758 | R-S1-Ferritin | His8-3c-Khosta2-MZ190138 | HHHHHHHHGPLEVLFQGPNMTNICPFDQVFNKTQFPSVYAWERVRISDCVSDYTVLYNSSASFSTFK | 395 |
| | | RBD-GSGGSG-S1-Ferritin | CYGVSPTKLNDLCFSGVYADYFVVKGDHVHQIAPGQTGVIADYNYKLPSEFVGCILAWNTRTIDSKRG | |
| | | | FYYRLFRHGNIRPYERDTSNVPYNAAGGTCNQPGTHNCYEPLQDYGFTSTSGVGYQPFRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP759 | R-S1-Ferritin | His8-3c-Khosta-2 RBD- | HHHHHHHHGPLEVLFQGPNMTNICPFDQVFNKTQFPSVYAWERVRISDCVSDYTVLYNSSASFSTFK | 396 |
| | | T498W-GSGGSG-S1-Ferritin | CYGVSPTKLNDLCFSGVYADYFWKGDHVHQIAPGQTGVIADYNYKLPSEFVGCILAWNTRTIDSKRG | |
| | | | FYYRLFRHGNIRPYERDTSNVPYNAAGGTCNQPGTHNCYEPLQDYGFWSTSGVGYQPFRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP760 | R-S1-Ferritin | His8-3c-Khosta-2 RBD- | HHHHHHHHGPLEVLFQGPNMTNICPFDQVFNKTQFPSVYAWERVRISDCVSDYTVLYNSSASFSTFK | 397 |
| | | Q493Y-T498W-GSGGSG-S1- | CYGVSPTKLNDLCFSGVYADYFVVKGDHVHQIAPGQTGVIADYNYKLPSEFVGCILAWNTRTIDSKRG | |
| | | Ferritin | FYYRLFRHGNIRPYERDTSNVPYNAAGGTCNQPGTHNCYEPLYDYGFWSTSGVGYQPFRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP761 | R-S1-Ferritin | His8-3c-KY352407_ | HHHHHHHHGPLEVLFQGPNITNLCPFGQVFNASNFPSVYAWERLRISDCVADYAVLYNSSSSFSTFKC | 398 |
| | | BtKY72_bat_Kenya RBD- | YGVSPTKLNDLCFSSVYADYFWVKGDDVRQIAPAQTGVIADYNYKLPDDFTGCVLAWNTNSVDSKSG | |
| | | GSGGSG-S1-Ferritin | NNFYYRLFRHGKIKPYERDISNVLYNSAGGTCSSISQLGCYEPLKSYGFTPTVGVGYQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP762 | R-S1-Ferritin | His8-3c-BtKY72 RBD-T498W- | HHHHHHHHGPLEVLFQGPNITNLCPFGQVFNASNFPSVYAWERLRISDCVADYAVLYNSSSSFSTFKC | 399 |
| | | GSGGSG-S1-Ferritin | YGVSPTKLNDLCFSSVYADYFWVKGDDVRQIAPAQTGVIADYNYKLPDDFTGCVLAWNTNSVDSKSG | |
| | | | NNFYYRLFRHGKIKPYERDISNVLYNSAGGTCSSISQLGCYEPLKSYGFWPTVGVGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP763 | R-S1-Ferritin | His8-3c-BtKY72 RBD-K493Y- | HHHHHHHHGPLEVLFQGPNITNLCPFGQVFNASNFPSVYAWERLRISDCVADYAVLYNSSSSFSTFKC | 400 |
| | | T498W-GSGGSG-S1- | YGVSPTKLNDLCFSSVYADYFWVKGDDVRQIAPAQTGVIADYNYKLPDDFTGCVLAWNTNSVDSKSG | |
| | | Ferritin | NNFYYRLFRHGKIKPYERDISNVLYNSAGGTCSSISQLGCYEPLYSYGFWPTVGVGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP764 | R-S1-Ferritin | His8-3c-RaTG15_Ra7909 RBD- | HHHHHHHHGPLEVLFQGPNITNLCPFSQVFNATRFPSVYAWTRERISNCIADYSVLYNSTSFSTFRCY | 401 |
| | | GSGGSG-S1-Ferritin | GVSPTKLNDLCFSNVYADSMVVRGDEVRQIAPSQTGVIADYNYKLPDDFTGCVIAWNSKAKDENGQY | |
| | | | FYRLFRKSKLLPFQRDVSNVTYGSGKNDGCNPSEADCYWPLLKYGFTGSVSQDYQPYRVVVLSFELL | |
| | | | NAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLKSFTVEKGIYQTSNFRVQPTESIVRFPNITNLCPFG | |
| | | | EVFNATRFASVYAWNRKRISNCVADYSVLYNSASFSTFKCYGVSPTKLNDLCFTNVYADSFVIRGDEV | |
| | | | RQIAPGQTGKIADYNYKLPDDFTGCVIAWNSNNLDSKVGGNYNYLYRLFRKSNLKPFERDISTEIYQAG | |
| | | | STPCNGVEGFNCYFPLQSYGFQPTNGVGYQPYRVVVLSFELLHAPATVCGPKKSTNLVKNKCVNFNF | |
| | | | NGLTGTGVLTESNKKFLPFQQFGRDIADTTDAVRDPQTLEILDITPCSFGGVSVITPGTNTSNQVAVLY | |
| | | | QDVNCTEVPVAIHADQLTPTWRVYSTGSNVFQTRAGCLIGAEHVNNSYECDIPIGAGICASYQTGGSQ | |
| | | | SIIAYTGSGGGGESQVRQQFSKDIEKLLNEQVNKEMQSSNLYMSMSSWCYTHSLDGAGLFLFDHAAE | |
| | | | EYEHAKKLIIFLNENNVPVQLTSISAPEHKFEGLTQIFQKAYEHEQHISESINNIVDHAIKSKDHATFNFLQ | |
| | | | WYVAEQHEEEVLFKDILDKIELIGNENHGLYLADQYVKGIAKSRKSGS | |
| pCoVP765 | R-S1-Ferritin | His8-3c-HKU3-1_ | HHHHHHHHGPLEVLFQGPNITNRCPFDKVFNATRFPNVYAWERTKISDCVADYTVLYNSTSFSTFKCY | 402 |
| | | DQ022305.2005 RBD-GSGGSG- | GVSPSKLIDLCFTSVYADTFLIRSSEVRQVAPGETGVIADYNYKLPDDFTGCVIAWNTAKHDTGNYYYR | |
| | | S1-Ferritin | SHRKTKLKPFERDLSSDDGNGVYTLSTYDFNPNVPVAYQATRVVVLSFELLNAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLKSFTVEKGIYQTSNFRVQPTESIVRFPNITNLCPFGEVFNATRFASVYAWNRKR | |
| | | | ISNCVADYSVLYNSASFSTFKCYGVSPTKLNDLCFTNVYADSFVIRGDEVRQIAPGQTGKIADYNYKLP | |
| | | | DDFTGCVIAWNSNNLDSKVGGNYNYLYRLFRKSNLKPFERDISTEIYQAGSTPCNGVEGFNCYFPLQS | |
| | | | YGFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTGGSQSIIAYTGSGGGGESQVRQ | |
| | | | QFSKDIEKLLNEQVNKEMQSSNLYMSMSSWCYTHSLDGAGLFLFDHAAEEYEHAKKLIIFLNENNVPV | |
| | | | QLTSISAPEHKFEGLTQIFQKAYEHEQHISESINNIVDHAIKSKDHATFNFLQWYVAEQHEEEVLFKDILD | |
| | | | KIELIGNENHGLYLADQYVKGIAKSRKSGS | |
| pCoVP766 | R-S1-Ferritin | His8-3c-RhGB01-MW719567 | HHHHHHHHGPLEVLFQGPNVTQLCPFSDVFNRTMFPSVYAWERMSIRDCVADYAVLYNSSISFSTFE | 403 |
| | | RBD-GSGGSG-S1-Ferritin | CYGVSPTKLNDLCFSSVYADYFVVRGDDVKQIAPGKTGVIADYNYKLPDDFVGCVLAWNSRHVDNKQ | |
| | | | GFYYRLFRHGKIKPYQRDMSRELYSPSGTPCTVVGFNCYDPLAHYAFASPGIGIGYQPFRVVVLSFQL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP767 | R-S1-Ferritin | His8-3c-bioinformatically | HHHHHHHHGPLEVLFQGPNITNLCPFDEVFNATRFPSVYAWERKKISNCVADYSVLYNSTSFSTFKCY | 404 |
| | | derived Cluster1_RBD (SARS- | GVSPSKLNDLCFTSVYADSFVIRGDEVRQIAPGQTGVIADYNYKLPDDFTGCVIAWNTANQDSGEGGN | |
| | | related)-GSGGSG-S1-Ferritin | PNYYYRSSRKSKLKPFERDLSSDEYNVGSQPCNRMGPNCYRTLSTYDFYPTVGVGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDE | |
| | | | VRQIAPGQTGKIADYNYKLPDDFTGCVIAWNSNNLDSKVGGNYNYLYRLFRKSNLKPFERDISTEIYQA | |
| | | | GSTPCNGVEGFNCYFPLQSYGFQPTNGVGYQPYRVVVLSFELLHAPATVCGPKKSTNLVKNKCVNFN | |
| | | | FNGLTGTGVLTESNKKFLPFQQFGRDIADTTDAVRDPQTLEILDITPCSFGGVSVITPGTNTSNQVAVL | |
| | | | YQDVNCTEVPVAIHADQLTPTWRVYSTGSNVFQTRAGCLIGAEHVNNSYECDIPIGAGICASYQTGGS | |
| | | | QSIIAYTGSGGGGESQVRQQFSKDIEKLLNEQVNKEMQSSNLYMSMSSWCYTHSLDGAGLFLFDHAA | |
| | | | EEYEHAKKLIIFLNENNVPVQLTSISAPEHKFEGLTQIFQKAYEHEQHISESINNIVDHAIKSKDHATFNFL | |
| | | | QWYVAEQHEEEVLFKDILDKIELIGNENHGLYLADQYVKGIAKSRKSGS | |
| pCoVP768 | R-S1-Ferritin | His8-3c-bioinformatically | HHHHHHHHGPLEVLFQGPNLPDCNIEEWLTAKSVPSPLNWERKTFSNCNFNLSSLLRFVQADSFSCN | 405 |
| | | derived Cluster2_RBD (OC43- | NIDASKVYGMCFGSISVDKFAIPNSRQVDLQLGNSGFLQSFNYKIDTTATSCQLYYSLPANNVTINNYN | |
| | | related)-GSGGSG-S1-Ferritin | PSSWNRRYGFNDDTVFKPRPAGVLTNHDVVYSQQCFKVPNTFCPCKNNGSCVSSGKGTTNGSGTC | |
| | | | PAGTNYRTCENTTWCACSCTPDPITASGPRKCPQKKSLVGVGEHCSGLGVDSDYCGNVSCLCSTDA | |
| | | | FLGWSADSCLVNDRCNIFANIILNGINSGTTCSTGSGGSGSSQCVNLTTRTQLPPAYTNSFTRGVYYP | |
| | | | DKVFRSSVLHSTQDLFLPFFSNVTWFHAIHVSGTNGTKRFDNPVLPFNDGVYFASTEKSNIIRGWIFGT | |
| | | | TLDSKTQSLLIVNNATNVVIKVCEFQFCNDPFLGVYYHKNNKSWMESEFRVYSSANNCTFEYVSQPFL | |
| | | | MDLEGKQGNFKNLREFVFKNIDGYFKIYSKHTPINLVRDLPQGFSALEPLVDLPIGINITRFQTLLALHRS | |
| | | | YLTPGDSSSGWTAGAAAYYVGYLQPRTFLLKYNENGTITDAVDCALDPLSETKCTLKSFTVEKGIYQT | |
| | | | SNFRVQPTESIVRFPNITNLCPFGEVFNATRFASVYAWNRKRISNCVADYSVLYNSASFSTFKCYGVSP | |
| | | | TKLNDLCFTNVYADSFVIRGDEVRQIAPGQTGKIADYNYKLPDDFTGCVIAWNSNNLDSKVGGNYNYL | |
| | | | YRLFRKSNLKPFERDISTEIYQAGSTPCNGVEGFNCYFPLQSYGFQPTNGVGYQPYRVVVLSFELLHA | |
| | | | PATVCGPKKSTNLVKNKCVNFNFNGLTGTGVLTESNKKFLPFQQFGRDIADTTDAVRDPQTLEILDITP | |
| | | | CSFGGVSVITPGTNTSNQVAVLYQDVNCTEVPVAIHADQLTPTWRVYSTGSNVFQTRAGCLIGAEHVN | |
| | | | NSYECDIPIGAGICASYQTGGSQSIIAYTGSGGGGESQVRQQFSKDIEKLLNEQVNKEMQSSNLYMSM | |
| | | | SSWCYTHSLDGAGLFLFDHAAEEYEHAKKLIIFLNENNVPVQLTSISAPEHKFEGLTQIFQKAYEHEQHI | |
| | | | SESINNIVDHAIKSKDHATFNFLQWYVAEQHEEEVLFKDILDKIELIGNENHGLYLADQYVKGIAKSRKS | |
| | | | GS | |
| pCoVP769 | R-S1-Ferritin | His8-3c-bioinformatically | HHHHHHHHGPLEVLFQGPQGVECDFSKLFKAAAPQIYNFSRLVFTNCNYNLTKLLSLFQVDEFSCHQI | 406 |
| | | derived Cluster3_RBD (MERS- | SPSALASGCYSSLTVDYFAYPLSMKSYLQIGSAGEISQFNYKQDFSNPTCRILASVPANVSITKPDKYIYI | |
| | | related)-GSGGSG-S1-Ferritin | SQCYSLSAYSDVDVPHYVNPGQYTSIPCLYLTSSGFDEDGQYFRYNLTQFEGQNYLVATGVTVSMTD | |
| | | | NLQMVFVITVQYGTDTNSVCPGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLKSFTVEKGIYQTSNFRVQPTESIV | |
| | | | RFPNITNLCPFGEVFNATRFASVYAWNRKRISNCVADYSVLYNSASFSTFKCYGVSPTKLNDLCFTNV | |
| | | | YADSFVIRGDEVRQIAPGQTGKIADYNYKLPDDFTGCVIAWNSNNLDSKVGGNYNYLYRLFRKSNLKP | |
| | | | FERDISTEIYQAGSTPCNGVEGFNCYFPLQSYGFQPTNGVGYQPYRVVVLSFELLHAPATVCGPKKST | |
| | | | NLVKNKCVNFNFNGLTGTGVLTESNKKFLPFQQFGRDIADTTDAVRDPQTLEILDITPCSFGGVSVITP | |
| | | | GTNTSNQVAVLYQDVNCTEVPVAIHADQLTPTWRVYSTGSNVFQTRAGCLIGAEHVNNSYECDIPIGA | |
| | | | GICASYQTGGSQSIIAYTGSGGGGESQVRQQFSKDIEKLLNEQVNKEMQSSNLYMSMSSWCYTHSLD | |
| | | | GAGLFLFDHAAEEYEHAKKLIIFLNENNVPVQLTSISAPEHKFEGLTQIFQKAYEHEQHISESINNIVDHAI | |
| | | | KSKDHATFNFLQWYVAEQHEEEVLFKDILDKIELIGNENHGLYLADQYVKGIAKSRKSGS | |
| pCoVP770 | R-S1-Ferritin | His8-3c-MER1 (ErinaceusCoV/ | HHHHHHHHGPLEVLFQGPELTECDLDVLFKNDAPIIANYSRRVFTNCNYNLTKLLSLVQVDEFVCHKTT | 407 |
| | | 2012-174/GER/2012) RBD- | PEALATGCYSSLTVDWFALPFSMKSTLAIGSAEAISMFNYNQDYSNPTCRIHAAVTANVSTALNFTANA | |
| | | GSGGSG-S1-Ferritin | NYAYISRCQGVDGKPILLQPGQMTNIACRSGVLARPSDADYFGYSFQGRNYYLGRKSYKPKTDEGDV | |
| | | | QMVYVITPKYDKGPDTVCPLKGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLKSFTVEKGIYQTSNFRVQPTESIV | |
| | | | RFPNITNLCPFGEVFNATRFASVYAWNRKRISNCVADYSVLYNSASFSTFKCYGVSPTKLNDLCFTNV | |
| | | | YADSFVIRGDEVRQIAPGQTGKIADYNYKLPDDFTGCVIAWNSNNLDSKVGGNYNYLYRLFRKSNLKP | |
| | | | FERDISTEIYQAGSTPCNGVEGFNCYFPLQSYGFQPTNGVGYQPYRVVVLSFELLHAPATVCGPKKST | |
| | | | NLVKNKCVNFNFNGLTGTGVLTESNKKFLPFQQFGRDIADTTDAVRDPQTLEILDITPCSFGGVSVITP | |
| | | | GTNTSNQVAVLYQDVNCTEVPVAIHADQLTPTWRVYSTGSNVFQTRAGCLIGAEHVNNSYECDIPIGA | |
| | | | GICASYQTGGSQSIIAYTGSGGGGESQVRQQFSKDIEKLLNEQVNKEMQSSNLYMSMSSWCYTHSLD | |
| | | | GAGLFLFDHAAEEYEHAKKLIIFLNENNVPVQLTSISAPEHKFEGLTQIFQKAYEHEQHISESINNIVDHAI | |
| | | | KSKDHATFNFLQWYVAEQHEEEVLFKDILDKIELIGNENHGLYLADQYVKGIAKSRKSGS | |
| pCoVP771 | R-S1-Ferritin | His8-3c-MER2 (Neoromicia/ | HHHHHHHHGPLEVLFQGPVYPDCNFTELFRERAPTIMQYKREVFTRCNYNLSLLLSLVQVDEFVCDKI | 408 |
| | | 5038) RBD-GSGGSG-S1- | TPEALATGCYSSLTVDWFAFPYAWKSYLAIGSADRIVRFNYNQDYSNPSCRIHSKVNSSIGISYAGAYS | |
| | | Ferritin | YITNCNYGATNKDDVVKPGGRASQQCITGALNSPTTGQLWAYNFGGVPYRVSRLTYTDHLSDPLDMV | |
| | | | YVITVKYEPGAETVCPKQGSGGSGSSQCVNLTTRTQLPPAYTNSFTRGVYYPDKVFRSSVLHSTQDL | |
| | | | FLPFFSNVTWFHAIHVSGTNGTKRFDNPVLPFNDGVYFASTEKSNIIRGWIFGTTLDSKTQSLLIVNNAT | |
| | | | NVVIKVCEFQFCNDPFLGVYYHKNNKSWMESEFRVYSSANNCTFEYVSQPFLMDLEGKQGNFKNLR | |
| | | | EFVFKNIDGYFKIYSKHTPINLVRDLPQGFSALEPLVDLPIGINITRFQTLLALHRSYLTPGDSSSGWTAG | |
| | | | AAAYYVGYLQPRTFLLKYNENGTITDAVDCALDPLSETKCTLKSFTVEKGIYQTSNFRVQPTESIVRFPN | |
| | | | ITNLCPFGEVFNATRFASVYAWNRKRISNCVADYSVLYNSASFSTFKCYGVSPTKLNDLCFTNVYADS | |
| | | | FVIRGDEVRQIAPGQTGKIADYNYKLPDDFTGCVIAWNSNNLDSKVGGNYNYLYRLFRKSNLKPFERDI | |
| | | | STEIYQAGSTPCNGVEGFNCYFPLQSYGFQPTNGVGYQPYRVVVLSFELLHAPATVCGPKKSTNLVK | |
| | | | NKCVNFNFNGLTGTGVLTESNKKFLPFQQFGRDIADTTDAVRDPQTLEILDITPCSFGGVSVITPGTNT | |
| | | | SNQVAVLYQDVNCTEVPVAIHADQLTPTWRVYSTGSNVFQTRAGCLIGAEHVNNSYECDIPIGAGICA | |
| | | | SYQTGGSQSIIAYTGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP772 | R-S1-Ferritin | His8-3c-MER3 (HKU4 SM3A) | HHHHHHHHGPLEVLFQGPQATECDFSPMLTGVAPQVYNFKRLVFSNCNYNLTKLLSLFAVDEFSCNGI | 409 |
| | | RBD-GSGGSG-S1-Ferritin | SPDAIARGCYSTLTVDYFAYPLSMKSYIRPGSAGNIPLYNYKQSFANPTCRVMASVPANVTITKPDAYG | |
| | | | YISKCSRLTGANQDVETPLYINPGEYSICRDFSPGGFSEDGQVFKRTLTQFEGGGLLIGVGTRVPMTD | |
| | | | NLQMSFIIFVQYGTGTDSVCPMLGSGGSGSSQCVNLTTRTQLPPAYTNSFTRGVYYPDKVFRSSVLH | |
| | | | STQDLFLPFFSNVTWFHAIHVSGTNGTKRFDNPVLPFNDGVYFASTEKSNIIRGWIFGTTLDSKTQSLLI | |
| | | | VNNATNVVIKVCEFQFCNDPFLGVYYHKNNKSWMESEFRVYSSANNCTFEYVSQPFLMDLEGKQGN | |
| | | | FKNLREFVFKNIDGYFKIYSKHTPINLVRDLPQGFSALEPLVDLPIGINITRFQTLLALHRSYLTPGDSSSG | |
| | | | WTAGAAAYYVGYLQPRTFLLKYNENGTITDAVDCALDPLSETKCTLKSFTVEKGIYQTSNFRVQPTESI | |
| | | | VRFPNITNLCPFGEVFNATRFASVYAWNRKRISNCVADYSVLYNSASFSTFKCYGVSPTKLNDLCFTN | |
| | | | VYADSFVIRGDEVRQIAPGQTGKIADYNYKLPDDFTGCVIAWNSNNLDSKVGGNYNYLYRLFRKSNLK | |
| | | | PFERDISTEIYQAGSTPCNGVEGFNCYFPLQSYGFQPTNGVGYQPYRVVVLSFELLHAPATVCGPKKS | |
| | | | TNLVKNKCVNFNFNGLTGTGVLTESNKKFLPFQQFGRDIADTTDAVRDPQTLEILDITPCSFGGVSVITP | |
| | | | GTNTSNQVAVLYQDVNCTEVPVAIHADQLTPTWRVYSTGSNVFQTRAGCLIGAEHVNNSYECDIPIGA | |
| | | | GICASYQTGGSQSIIAYTGSGGGGESQVRQQFSKDIEKLLNEQVNKEMQSSNLYMSMSSWCYTHSLD | |
| | | | GAGLFLFDHAAEEYEHAKKLIIFLNENNVPVQLTSISAPEHKFEGLTQIFQKAYEHEQHISESINNIVDHAI | |
| | | | KSKDHATFNFLQWYVAEQHEEEVLFKDILDKIELIGNENHGLYLADQYVKGIAKSRKSGS | |
| pCoVP773 | R-S1-Ferritin | His8-3c-MER4 (BatCoV-Ita2 | HHHHHHHHGPLEVLFQGPQGVECDFSKLFKAAPPQIYNFSRLVFTNCNYNLTKLLSLFHVSEFSCHQV | 410 |
| | | 206645-63) RBD-GSGGSG- | SPSALASGCYSSLTVDYFAYPLYLASYLQQGSTGEIAQYNYKQDFSNPTCRILASVPANVSIPKPDKYI | |
| | | S1-Ferritin | WLSQCYSFSAYSGDIPHYVQPGQYTPCLYLTSSGFDKSYQTNRDMQNKMAATGVTSSMTDNLQMAF | |
| | | | VISVQYGTDTNSVCPMGSGGSGSSQCVNLTTRTQLPPAYTNSFTRGVYYPDKVFRSSVLHSTQDLFL | |
| | | | PFFSNVTWFHAIHVSGTNGTKRFDNPVLPFNDGVYFASTEKSNIIRGWIFGTTLDSKTQSLLIVNNATN | |
| | | | WIKVCEFQFCNDPFLGVYYHKNNKSWMESEFRVYSSANNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLKSFTVEKGIYQTSNFRVQPTESIVRFPNIT | |
| | | | NLCPFGEVFNATRFASVYAWNRKRISNCVADYSVLYNSASFSTFKCYGVSPTKLNDLCFTNVYADSFV | |
| | | | IRGDEVRQIAPGQTGKIADYNYKLPDDFTGCVIAWNSNNLDSKVGGNYNYLYRLFRKSNLKPFERDIST | |
| | | | EIYQAGSTPCNGVEGFNCYFPLQSYGFQPTNGVGYQPYRVVVLSFELLHAPATVCGPKKSTNLVKNK | |
| | | | CVNFNFNGLTGTGVLTESNKKFLPFQQFGRDIADTTDAVRDPQTLEILDITPCSFGGVSVITPGTNTSN | |
| | | | QVAVLYQDVNCTEVPVAIHADQLTPTWRVYSTGSNVFQTRAGCLIGAEHVNNSYECDIPIGAGICASY | |
| | | | QTGGSQSIIAYTGSGGGGESQVRQQFSKDIEKLLNEQVNKEMQSSNLYMSMSSWCYTHSLDGAGLF | |
| | | | LFDHAAEEYEHAKKLIIFLNENNVPVQLTSISAPEHKFEGLTQIFQKAYEHEQHISESINNIVDHAIKSKDH | |
| | | | ATFNFLQWYVAEQHEEEVLFKDILDKIELIGNENHGLYLADQYVKGIAKSRKSGS | |
| pCoVP774 | R-S1-Ferritin | His8-3c-MER5 (BatCoV-Ita1 | HHHHHHHHGPLEVLFQGPQGVECDFSKLFKAAPPQIYNFSRLVFTNCNYNLTKLLSLFHVSEFSCHQV | 411 |
| | | 206645-40) RBD-GSGGSG- | SPSALASGCYSSLTVDYFAYPLYLASYLQQGSTGEIAQYNYKQDFSNPTCRILASVPANVSIPKPDKYI | |
| | | S1-Ferritin | WLSQCYSFSAYSGDVPHYVLPGQYTPCLYLTSSGFDNSYQTNRDFQNKMAATGVISSMTDNLQMAF | |
| | | | VISVQYGTDTNSVCPMGSGGSGSSQCVNLTTRTQLPPAYTNSFTRGVYYPDKVFRSSVLHSTQDLFL | |
| | | | PFFSNVTWFHAIHVSGTNGTKRFDNPVLPFNDGVYFASTEKSNIIRGWIFGTTLDSKTQSLLIVNNATN | |
| | | | WIKVCEFQFCNDPFLGVYYHKNNKSWMESEFRVYSSANNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLKSFTVEKGIYQTSNFRVQPTESIVRFPNIT | |
| | | | NLCPFGEVFNATRFASVYAWNRKRISNCVADYSVLYNSASFSTFKCYGVSPTKLNDLCFTNVYADSFV | |
| | | | IRGDEVRQIAPGQTGKIADYNYKLPDDFTGCVIAWNSNNLDSKVGGNYNYLYRLFRKSNLKPFERDIST | |
| | | | EIYQAGSTPCNGVEGFNCYFPLQSYGFQPTNGVGYQPYRVVVLSFELLHAPATVCGPKKSTNLVKNK | |
| | | | CVNFNFNGLTGTGVLTESNKKFLPFQQFGRDIADTTDAVRDPQTLEILDITPCSFGGVSVITPGTNTSN | |
| | | | QVAVLYQDVNCTEVPVAIHADQLTPTWRVYSTGSNVFQTRAGCLIGAEHVNNSYECDIPIGAGICASY | |
| | | | QTGGSQSIIAYTGSGGGGESQVRQQFSKDIEKLLNEQVNKEMQSSNLYMSMSSWCYTHSLDGAGLF | |
| | | | LFDHAAEEYEHAKKLIIFLNENNVPVQLTSISAPEHKFEGLTQIFQKAYEHEQHISESINNIVDHAIKSKDH | |
| | | | ATFNFLQWYVAEQHEEEVLFKDILDKIELIGNENHGLYLADQYVKGIAKSRKSGS | |
| pCoVP775 | R-S1-Ferritin | His8-3c-HKU5 RBD-GSGGSG- | HHHHHHHHGPLEVLFQGPTTQECDFTPMLTGTPPPIYNFKRLVFTNCNYNLTKLLSLFQVSEFSCHQV | 412 |
| | | S1-Ferritin | SPSSLATGCYSSLTVDYFAYSTDMSSYLQPGSAGAIVQFNYKQDFSNPTCRVLATVPQNLTTITKPSN | |
| | | | YAYLTECYKTSAYGKNYLYNAPGAYTPCLSLASRGFSTKYQSHSDGELTTTGYIYPVTGNLQMAFIISV | |
| | | | QYGTDTNSVCPMGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLKSFTVEKGIYQTSNFRVQPTESIVRFPNITNLCP | |
| | | | FGEVFNATRFASVYAWNRKRISNCVADYSVLYNSASFSTFKCYGVSPTKLNDLCFTNVYADSFVIRGD | |
| | | | EVRQIAPGQTGKIADYNYKLPDDFTGCVIAWNSNNLDSKVGGNYNYLYRLFRKSNLKPFERDISTEIYQ | |
| | | | AGSTPCNGVEGFNCYFPLQSYGFQPTNGVGYQPYRVVVLSFELLHAPATVCGPKKSTNLVKNKCVNF | |
| | | | NFNGLTGTGVLTESNKKFLPFQQFGRDIADTTDAVRDPQTLEILDITPCSFGGVSVITPGTNTSNQVAV | |
| | | | LYQDVNCTEVPVAIHADQLTPTWRVYSTGSNVFQTRAGCLIGAEHVNNSYECDIPIGAGICASYQTGG | |
| | | | SQSIIAYTGSGGGGESQVRQQFSKDIEKLLNEQVNKEMQSSNLYMSMSSWCYTHSLDGAGLFLFDHA | |
| | | | AEEYEHAKKLIIFLNENNVPVQLTSISAPEHKFEGLTQIFQKAYEHEQHISESINNIVDHAIKSKDHATFNF | |
| | | | LQWYVAEQHEEEVLFKDILDKIELIGNENHGLYLADQYVKGIAKSRKSGS | |
| pCoVP776 | R-R-N-Ferritin | His8-3c-RBD from Omicron | HHHHHHHHGPLEVLFQGPNITNLCPFDEVFNATRFASVYAWNRKRISNCVADYSVLYNFAPFFAFKCY | 413 |
| | | BA.4/5-GSGGSG-RN-Ferritin | GVSPTKLNDLCFTNVYADSFVIRGNEVSQIAPGQTGNIADYNYKLPDDFTGCVIAWNSNKLDSKVGGN | |
| | | | YNYRYRLFRKSNLKPFERDISTEIYQAGNKPCNGVAGVNCYFPLQSYGFRPTYGVGHQPYRVVVLSFE | |
| | | | LLHAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP777 | R-R-N-Ferritin | His8-3c-RBD from Omicron | HHHHHHHHGPLEVLFQGPNITNLCPFDEVFNATRFASVYAWNRKRISNCVADYSVLYNFAPFFAFKCY | 414 |
| | | BA.2.12.1-GSGGSG-RN- | GVSPTKLNDLCFTNVYADSFVIRGNEVSQIAPGQTGNIADYNYKLPDDFTGCVIAWNSNKLDSKVGGN | |
| | | Ferritin | YNYQYRLFRKSNLKPFERDISTEIYQAGNKPCNGVAGFNCYFPLRSYGFRPTYGVGHQPYRVVVLSFE | |
| | | | LLHAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP778 | R-R-N-Ferritin | His8-3c-RBD from | HHHHHHHHGPLEVLFQGPNITQLCPFNEVFNITSFPSVYAWERMRITNCVADYSVLYNSSASFSTFQC | 415 |
| | | NC_014470.BM4831.BGR.2008- | YGVSPTKLNDLCFSSVYADYFWVKGDDVRQIAPAQTGVIADYNYKLPDDFTGCVIAWNTNSLDSSNEF | |
| | | GSGGSG-RN-Ferritin | FYRRFRHGKIKPYGRDLSNVLFNPSGGTCSAEGLNCYKPLASYGFTQSSGIGFQPYRVVVLSFELLNA | |
| | | | PATVCGPGSGGSGSSQCVNLTTRTQLPPAYTNSFTRGVYYPDKVFRSSVLHSTQDLFLPFFSNVTWF | |
| | | | HAIHVSGTNGTKRFDNPVLPFNDGVYFASTEKSNIIRGWIFGTTLDSKTQSLLIVNNATNVVIKVCEFQF | |
| | | | CNDPFLGVYYHKNNKSWMESEFRVYSSANNCTFEYVSQPFLMDLEGKQGNFKNLREFVFKNIDGYFK | |
| | | | IYSKHTPINLVRDLPQGFSALEPLVDLPIGINITRFQTLLALHRSYLTPGDSSSGWTAGAAAYYVGYLQP | |
| | | | RTFLLKYNENGTITDAVDCALDPLSETKCTLGSGGGGESQVRQQFSKDIEKLLNEQVNKEMQSSNLYM | |
| | | | SMSSWCYTHSLDGAGLFLFDHAAEEYEHAKKLIIFLNENNVPVQLTSISAPEHKFEGLTQIFQKAYEHE | |
| | | | QHISESINNIVDHAIKSKDHATFNFLQWYVAEQHEEEVLFKDILDKIELIGNENHGLYLADQYVKGIAKSR | |
| | | | KSGS | |
| pCoVP779 | R-R-N-Ferritin | His8-3c-BM48-31 RBD-T498W- | HHHHHHHHGPLEVLFQGPNITQLCPFNEVFNITSFPSVYAWERMRITNCVADYSVLYNSSASFSTFQC | 416 |
| | | GSGGSG-RN-Ferritin | YGVSPTKLNDLCFSSVYADYFVVKGDDVRQIAPAQTGVIADYNYKLPDDFTGCVIAWNTNSLDSSNEF | |
| | | | FYRRFRHGKIKPYGRDLSNVLFNPSGGTCSAEGLNCYKPLASYGFWQSSGIGFQPYRVVVLSFELLNA | |
| | | | PATVCGPGSGGSGSSQCVNLTTRTQLPPAYTNSFTRGVYYPDKVFRSSVLHSTQDLFLPFFSNVTWF | |
| | | | HAIHVSGTNGTKRFDNPVLPFNDGVYFASTEKSNIIRGWIFGTTLDSKTQSLLIVNNATNVVIKVCEFQF | |
| | | | CNDPFLGVYYHKNNKSWMESEFRVYSSANNCTFEYVSQPFLMDLEGKQGNFKNLREFVFKNIDGYFK | |
| | | | IYSKHTPINLVRDLPQGFSALEPLVDLPIGINITRFQTLLALHRSYLTPGDSSSGWTAGAAAYYVGYLQP | |
| | | | RTFLLKYNENGTITDAVDCALDPLSETKCTLGSGGGGESQVRQQFSKDIEKLLNEQVNKEMQSSNLYM | |
| | | | SMSSWCYTHSLDGAGLFLFDHAAEEYEHAKKLIIFLNENNVPVQLTSISAPEHKFEGLTQIFQKAYEHE | |
| | | | QHISESINNIVDHAIKSKDHATFNFLQWYVAEQHEEEVLFKDILDKIELIGNENHGLYLADQYVKGIAKSR | |
| | | | KSGS | |
| pCoVP780 | R-R-N-Ferritin | His8-3c-BM48-31 RBD-A493Y- | HHHIIHHHGPLEVLFQGPNITQLCPFNEVFNITSFPSVYAWERMRITNCVADYSVLYNSSASFSTFQC | 417 |
| | | T498W-GSGGSG-RN- | YGVSPTKLNDLCFSSVYADYFVVKGDDVRQIAPAQTGVIADYNYKLPDDFTGCVIAWNTNSLDSSNEF | |
| | | Ferritin | FYRRFRHGKIKPYGRDLSNVLFNPSGGTCSAEGLNCYKPLYSYGFWQSSGIGFQPYRVVVLSFELLNA | |
| | | | PATVCGPGSGGSGSSQCVNLTTRTQLPPAYTNSFTRGVYYPDKVFRSSVLHSTQDLFLPFFSNVTWF | |
| | | | HAIHVSGTNGTKRFDNPVLPFNDGVYFASTEKSNIIRGWIFGTTLDSKTQSLLIVNNATNVVIKVCEFQF | |
| | | | CNDPFLGVYYHKNNKSWMESEFRVYSSANNCTFEYVSQPFLMDLEGKQGNFKNLREFVFKNIDGYFK | |
| | | | IYSKHTPINLVRDLPQGFSALEPLVDLPIGINITRFQTLLALHRSYLTPGDSSSGWTAGAAAYYVGYLQP | |
| | | | RTFLLKYNENGTITDAVDCALDPLSETKCTLGSGGGGESQVRQQFSKDIEKLLNEQVNKEMQSSNLYM | |
| | | | SMSSWCYTHSLDGAGLFLFDHAAEEYEHAKKLIIFLNENNVPVQLTSISAPEHKFEGLTQIFQKAYEHE | |
| | | | QHISESINNIVDHAIKSKDHATFNFLQWYVAEQHEEEVLFKDILDKIELIGNENHGLYLADQYVKGIAKSR | |
| | | | KSGS | |
| pCoVP781 | R-R-N-Ferritin | His8-3c-RBD from KC881005. | HHHHHHHHGPLEVLFQGPNITNLCPFGEVFNATTFPSVYAWERKRISNCVADYSVLYNSTSFSTFKCY | 418 |
| | | RsSHC014-GSGGSG-RN- | GVSATKLNDLCFSNVYADSFVVKGDDVRQIAPGQTGVIADYNYKLPDDFLGCVLAWNTNSKDSSTSG | |
| | | Ferritin | NYNYLYRWVRRSKLNPYERDLSNDIYSPGGQSCSAVGPNCYNPLRPYGFFTTAGVGHQPYRVVVLSF | |
| | | | ELLNAPATVCGPGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLGSGGGGESQVRQQFSKDIEKLLNEQVNKEMQ | |
| | | | SSNLYMSMSSWCYTHSLDGAGLFLFDHAAEEYEHAKKLIIFLNENNVPVQLTSISAPEHKFEGLTQIFQ | |
| | | | KAYEHEQHISESINNIVDHAIKSKDHATFNFLQWYVAEQHEEEVLFKDILDKIELIGNENHGLYLADQYV | |
| | | | KGIAKSRKSGS | |
| pCoVP782 | R-R-N-Ferritin | His8-3c-RBD from KC881007. | HHHHHHHHGPLEVLFQGPNITNLCPFGEVFNATTFPSVYAWERKRISNCVADYSVLYNSTSFSTFKCY | 419 |
| | | Bat.WIV1-GSGGSG-RN- | GVSATKLNDLCFSNVYADSFVVKGDDVRQIAPGQTGVIADYNYKLPDDFTGCVLAWNTRNIDATQTGN | |
| | | Ferritin | YNYKYRSLRHGKLRPFERDISNVPFSPDGKPCTPPAFNCYWPLNDYGFYITNGIGYQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP783 | R-R-N-Ferritin | His8-3c-RBD from AVP78042_ | HHHHHHHHGPLEVLFQGPNITNVCPFHKVFNATRFPSVYAWERTKISDCIADYTVFYNSTSFSTFKCY | 420 |
| | | BatSARSlikecoronavirus- | GVSPSKLIDLCFTSVYADTFLIRFSEVRQVAPGQTGVIADYNYKLPDDFTGCVIAWNTAKQDTGHYFYR | |
| | | GSGGSG-RN-Ferritin | SHRSTKLKPFERDLSSDENGVRTLSTYDFNPNVPLEYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP784 | R-R-N-Ferritin | His8-3c-RBD from MZ937001_ | HHHHHHHHGPLEVLFQGPNITNLCPFGEVFNATTFASVYAWNRKRISNCVADYSVLYNSTSFSTFKCY | 421 |
| | | BANAL20103_Laos_2020- | GVSPTKLNDLCFTNVYADSFVVRGDEVRQIAPGQTGKIADYNYKLPDDFTGCVIAWNSNNLDSKVGG | |
| | | GSGGSG-RN-Ferritin | NYNYLYRLFRKSNLKPFERDISTEIYQAGSTPCNGVEGFNCYFPLQSYGFHPTNGVGYQPYRVVVLSF | |
| | | | ELLNAPATVCGPGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLGSGGGGESQVRQQFSKDIEKLLNEQVNKEMQ | |
| | | | SSNLYMSMSSWCYTHSLDGAGLFLFDHAAEEYEHAKKLIIFLNENNVPVQLTSISAPEHKFEGLTQIFQ | |
| | | | KAYEHEQHISESINNIVDHAIKSKDHATFNFLQWYVAEQHEEEVLFKDILDKIELIGNENHGLYLADQYV | |
| | | | KGIAKSRKSGS | |
| pCoVP785 | R-R-N-Ferritin | His8-3c-RBD from | HHHHHHHHGPLEVLFQGPNITNLCPFGEVFNATTFASVYAWNRKRISNCVADYSVLYNSTSFSTFKCY | 422 |
| | | MN996532.2013.Bat.RaTG13- | GVSPTKLNDLCFTNVYADSFVITGDEVRQIAPGQTGKIADYNYKLPDDFTGCVIAWNSKHIDAKEGGNF | |
| | | GSGGSG-RN-Ferritin | NYLYRLFRKANLKPFERDISTEIYQAGSKPCNGQTGLNCYYPLYRYGFYPTDGVGHQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP786 | R-R-N-Ferritin | His8-3c-RBD from MZ937004_ | HHHHHHHHGPLEVLFQGPNITNFCPFDKVFNATRFPNVYAWQRTKISDCIADYTVLYNSTSFSTFKCY | 423 |
| | | BANAL20247_Laos_2020- | GVSPSKLIDLCFTSVYADTFLIRFSEVRQIAPGETGVIADYNYKLPDDFTGCVLAWNTAQQDIGSYFYR | |
| | | GSGGSG-RN-Ferritin | SHRAVKLKPFERDLSSDENGVRTLSTYDFNPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP787 | R-R-N-Ferritin | His8-3c-BANAL-20-247 RBD- | HHHHHHHHGPLEVLFQGPNITNFCPFDKVFNATRFPNVYAWQRTKISDCIADYTVLYNSTSFSTFKCY | 424 |
| | | N498W-GSGGSG-RN- | GVSPSKLIDLCFTSVYADTFLIRFSEVRQIAPGETGVIADYNYKLPDDFTGCVLAWNTAQQDIGSYFYR | |
| | | Ferritin | SHRAVKLKPFERDLSSDENGVRTLSTYDFWPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP788 | R-R-N-Ferritin | His8-3c-BANAL-20-247 RBD- | HHHHHHHHGPLEVLFQGPNITNFCPFDKVFNATRFPNVYAWQRTKISDCIADYTVLYNSTSFSTFKCY | 425 |
| | | S493Y-N498W-GSGGSG- | GVSPSKLIDLCFTSVYADTFLIRFSEVRQIAPGETGVIADYNYKLPDDFTGCVLAWNTAQQDIGSYFYR | |
| | | RN-Ferritin | SHRAVKLKPFERDLSSDENGVRTLYTYDFWPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP789 | R-R-N-Ferritin | His8-3c-RBD from ATO98145_ | HHHHHHHHGPLEVLFQGPNITNLCPFDAVFNATRFPSVYAWERVKISNCVADYTAFYNSTSFSTFKCY | 426 |
| | | BatSARSlikecoronavirus- | GVSPSKLIDLCFTSVYADTFLIRFSEVRQVAPGETGVIADYNYKLPDDFTGCVIAWNTAKQDVGSYFYR | |
| | | GSGGSG-RN-Ferritin | SHRSSKLKPFERDLSSDENGVRTLSTYDFNPNVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP790 | R-R-N-Ferritin | His8-3c-RBD from JX993987. | HHHHHHHHGPLEVLFQGPNITNRCPFDKVFNATRFPSVYAWERTKISDCVADYTVLYNSTSFSTFKCY | 427 |
| | | 2011 .BatRp.Shaanxi2011- | GVSPSKLIDLCFTSVYADTFLIRSSEVRQVAPGETGVIADYNYKLPDDFTGCVIAWNTANQDQGQYYY | |
| | | GSGGSG-RN-Ferritin | RSSRKEKLKPFERDLSSDENGVYTLSTYDFYPSVPLDYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNWVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP791 | R-R-N-Ferritin | His8-3c-RBD from KJ473812. | HHHHHHHHGPLEVLFQGPNITNLCPFDKVFNATRFPSVYAWERTKISDCVADYTVFYNSTSFSTENCY | 428 |
| | | BtRfBetaCoV.HeB2013- | GVSPSKLIDLCFTSVYADTFLIRFSEVRQVAPGQTGVIADYNYKLPDDFTGCVIAWNTAKQDVGSYFYR | |
| | | GSGGSG-RN-Ferritin | SHRSSKLKPFERDLSSEENGVRTLSTYDFNQYVPLEYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP792 | R-R-N-Ferritin | His8-3c-RBD from DQ071615. | HHHHHHHHGPLEVLFQGPNITNRCPFDKVFNATRFPNVYAWERTKISDCVADYTVLYNSTSFSTFKCY | 429 |
| | | 2003.Bat.Rp3-GSGGSG- | GVSPSKLIDLCFTSVYADTFLIRSSEVRQVAPGETGVIADYNYKLPDDFTGCVIAWNTAKQDQGQYYY | |
| | | RN-Ferritin | RSHRKTKLKPFERDLSSDENGVRTLSTYDFYPSVPVAYQATRVVVLSFELLNAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLGSGGGGESQVRQQFSKDIEKLLNEQVNKEMQSSNLYMSMSSWCYTHSLDGA | |
| | | | GLFLFDHAAEEYEHAKKLIIFLNENNVPVQLTSISAPEHKFEGLTQIFQKAYEHEQHISESINNIVDHAIKS | |
| | | | KDHATFNFLQWYVAEQHEEEVLFKDILDKIELIGNENHGLYLADQYVKGIAKSRKSGS | |
| pCoVP793 | R-R-N-Ferritin | His8-3c-RBD from ACU31032_ | HHHHHHHHGPLEVLFQGPNITNRCPFDKVFNASRFPNVYAWERTKISDCVADYTVLYNSTSFSTFKCY | 430 |
| | | Rs_672_2006-GSGGSG- | GVSPSKLIDLCFTSVYADTFLIRSSEVRQVAPGETGVIADYNYKLPDDFTGCVIAWNTAKQDQGQYYY | |
| | | RN-Ferritin | RSSRKTKLKPFERDLTSDENGVRTLSTYDFYPNVPIEYQATRVVVLSFELLNAPATVCGPGSGGSGSS | |
| | | | QCVNLTTRTQLPPAYTNSFTRGVYYPDKVFRSSVLHSTQDLFLPFFSNVTWFHAIHVSGTNGTKRFDN | |
| | | | PVLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLGSGGGGESQVRQQFSKDIEKLLNEQVNKEMQSSNLYMSMSSWCYTHSLDGAG | |
| | | | LFLFDHAAEEYEHAKKLIIFLNENNVPVQLTSISAPEHKFEGLTQIFQKAYEHEQHISESINNIVDHAIKSK | |
| | | | DHATFNFLQWYVAEQHEEEVLFKDILDKIELIGNENHGLYLADQYVKGIAKSRKSGS | |
| pCoVP794 | R-R-N-Ferritin | His8-3c-RBD from MZ081380_ | HHHHHHHHGPLEVLFQGPNITNLCPFSQVFNATRFPSVYAWTRERISNCIADYSVLYNSTSFSTFRCY | 431 |
| | | bat_Yunnan_RsYN04_2020 | GVSPTKLNDLCFSNVYADSMVVRGDEVRQIAPSQTGVIADYNYKLPDDFTGCVIAWNSKAKDENGQY | |
| | | GSGGSG-RN-Ferritin | FYRLFRKSKLLPFQRDVSNVTYGSGKNDGCNPSEADCYWPLLKYGFTSSVSQDYQPYRVVVLSFELL | |
| | | | NAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLGSGGGGESQVRQQFSKDIEKLLNEQVNKEMQSSN | |
| | | | LYMSMSSWCYTHSLDGAGLFLFDHAAEEYEHAKKLIIFLNENNVPVQLTSISAPEHKFEGLTQIFQKAY | |
| | | | EHEQHISESINNIVDHAIKSKDHATFNFLQWYVAEQHEEEVLFKDILDKIELIGNENHGLYLADQYVKGIA | |
| | | | KSRKSGS | |
| pCoVP795 | R-R-N-Ferritin | His8-3c-RsYN04 RBD-T498W- | HHHHHHHHGPLEVLFQGPNITNLCPFSQVFNATRFPSVYAWTRERISNCIADYSVLYNSTSFSTFRCY | 432 |
| | | GSGGSG-RN-Ferritin | GVSPTKLNDLCFSNVYADSMVVRGDEVRQIAPSQTGVIADYNYKLPDDFTGCVIAWNSKAKDENGQY | |
| | | | FYRLFRKSKLLPFQRDVSNVTYGSGKNDGCNPSEADCYWPLLKYGFWSSVSQDYQPYRVVVLSFELL | |
| | | | NAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLGSGGGGESQVRQQFSKDIEKLLNEQVNKEMQSSN | |
| | | | LYMSMSSWCYTHSLDGAGLFLFDHAAEEYEHAKKLIIFLNENNVPVQLTSISAPEHKFEGLTQIFQKAY | |
| | | | EHEQHISESINNIVDHAIKSKDHATFNFLQWYVAEQHEEEVLFKDILDKIELIGNENHGLYLADQYVKGIA | |
| | | | KSRKSGS | |
| pCoVP796 | R-R-N-Ferritin | His8-3c-RsYN04 RBD-L493Y- | HHHHHHHHGPLEVLFQGPNITNLCPFSQVFNATRFPSVYAWTRERISNCIADYSVLYNSTSFSTFRCY | 433 |
| | | T498W-GSGGSG-RN- | GVSPTKLNDLCFSNVYADSMVVRGDEVRQIAPSQTGVIADYNYKLPDDFTGCVIAWNSKAKDENGQY | |
| | | Ferritin | FYRLFRKSKLLPFQRDVSNVTYGSGKNDGCNPSEADCYWPLYKYGFWSSVSQDYQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP797 | R-R-N-Ferritin | His8-3c-Khosta2-MZ190138 | HHHHHHHHGPLEVLFQGPNMTNICPFDQVFNKTQFPSVYAWERVRISDCVSDYTVLYNSSASFSTFK | 434 |
| | | RBD-GSGGSG-RN-Ferritin | CYGVSPTKLNDLCFSGVYADYFVVKGDHVHQIAPGQTGVIADYNYKLPSEFVGCILAWNTRTIDSKRG | |
| | | | FYYRLFRHGNIRPYERDTSNVPYNAAGGTCNQPGTHNCYEPLQDYGFTSTSGVGYQPFRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP798 | R-R-N-Ferritin | His8-3c-Khosta-2 RBD- | HHHHHHHHGPLEVLFQGPNMTNICPFDQVFNKTQFPSVYAWERVRISDCVSDYTVLYNSSASFSTFK | 435 |
| | | T498W-GSGGSG-RN-Ferritin | CYGVSPTKLNDLCFSGVYADYFVVKGDHVHQIAPGQTGVIADYNYKLPSEFVGCILAWNTRTIDSKRG | |
| | | | FYYRLFRHGNIRPYERDTSNVPYNAAGGTCNQPGTHNCYEPLQDYGFWSTSGVGYQPFRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP799 | R-R-N-Ferritin | His8-3c-Khosta-2 RBD- | HHHHHHHHGPLEVLFQGPNMTNICPFDQVFNKTQFPSVYAWERVRISDCVSDYTVLYNSSASFSTFK | 436 |
| | | Q493Y-T498W-GSGGSG-RN- | CYGVSPTKLNDLCFSGVYADYFWVKGDHVHQIAPGQTGVIADYNYKLPSEFVGCILAWNTRTIDSKRG | |
| | | Ferritin | FYYRLFRHGNIRPYERDTSNVPYNAAGGTCNQPGTHNCYEPLYDYGFWSTSGVGYQPFRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP800 | R-R-N-Ferritin | His8-3c-KY352407_ | HHHHHHHHGPLEVLFQGPNITNLCPFGQVFNASNFPSVYAWERLRISDCVADYAVLYNSSSSFSTFKC | 437 |
| | | BtKY72_bat_Kenya RBD- | YGVSPTKLNDLCFSSVYADYFWVKGDDVRQIAPAQTGVIADYNYKLPDDFTGCVLAWNTNSVDSKSG | |
| | | GSGGSG-RN-Ferritin | NNFYYRLFRHGKIKPYERDISNVLYNSAGGTCSSISQLGCYEPLKSYGFTPTVGVGYQPYRVVVLSFEL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP801 | R-R-N-Ferritin | His8-3c-BtKY72 RBD-T498W- | HHHHHHHHGPLEVLFQGPNITNLCPFGQVFNASNFPSVYAWERLRISDCVADYAVLYNSSSSFSTFKC | 438 |
| | | GSGGSG-RN-Ferritin | YGVSPTKLNDLCFSSVYADYFVVKGDDVRQIAPAQTGVIADYNYKLPDDFTGCVLAWNTNSVDSKSG | |
| | | | NNFYYRLFRHGKIKPYERDISNVLYNSAGGTCSSISQLGCYEPLKSYGFWPTVGVGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP802 | R-R-N-Ferritin | His8-3c-BtKY72 RBD-K493Y- | HHHHHHHHGPLEVLFQGPNITNLCPFGQVFNASNFPSVYAWERLRISDCVADYAVLYNSSSSFSTFKC | 439 |
| | | T498W-GSGGSG-RN- | YGVSPTKLNDLCFSSVYADYFVVKGDDVRQIAPAQTGVIADYNYKLPDDFTGCVLAWNTNSVDSKSG | |
| | | Ferritin | NNFYYRLFRHGKIKPYERDISNVLYNSAGGTCSSISQLGCYEPLYSYGFWPTVGVGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP803 | R-R-N-Ferritin | His8-3c-RaTG15_Ra7909 RBD- | HHHHHHHHGPLEVLFQGPNITNLCPFSQVFNATRFPSVYAWTRERISNCIADYSVLYNSTSFSTFRCY | 440 |
| | | GSGGSG-RN-Ferritin | GVSPTKLNDLCFSNVYADSMVVRGDEVRQIAPSQTGVIADYNYKLPDDFTGCVIAWNSKAKDENGQY | |
| | | | FYRLFRKSKLLPFQRDVSNVTYGSGKNDGCNPSEADCYWPLLKYGFTGSVSQDYQPYRVVVLSFELL | |
| | | | NAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLGSGGGGESQVRQQFSKDIEKLLNEQVNKEMQSSN | |
| | | | LYMSMSSWCYTHSLDGAGLFLFDHAAEEYEHAKKLIIFLNENNVPVQLTSISAPEHKFEGLTQIFQKAY | |
| | | | EHEQHISESINNIVDHAIKSKDHATFNFLQWYVAEQHEEEVLFKDILDKIELIGNENHGLYLADQYVKGIA | |
| | | | KSRKSGS | |
| pCoVP804 | R-R-N-Ferritin | His8-3c-HKU3-1_ | HHHHHHHHGPLEVLFQGPNITNRCPFDKVFNATRFPNVYAWERTKISDCVADYTVLYNSTSFSTFKCY | 441 |
| | | DQ022305.2005 RBD-GSGGSG- | GVSPSKLIDLCFTSVYADTFLIRSSEVRQVAPGETGVIADYNYKLPDDFTGCVIAWNTAKHDTGNYYYR | |
| | | RN-Ferritin | SHRKTKLKPFERDLSSDDGNGVYTLSTYDFNPNVPVAYQATRVVVLSFELLNAPATVCGPGSGGSGS | |
| | | | SQCVNLTTRTQLPPAYTNSFTRGVYYPDKVFRSSVLHSTQDLFLPFFSNVTWFHAIHVSGTNGTKRFD | |
| | | | NPVLPFNDGVYFASTEKSNIIRGWIFGTTLDSKTQSLLIVNNATNVVIKVCEFQFCNDPFLGVYYHKNNK | |
| | | | SWMESEFRVYSSANNCTFEYVSQPFLMDLEGKQGNFKNLREFVFKNIDGYFKIYSKHTPINLVRDLPQ | |
| | | | GFSALEPLVDLPIGINITRFQTLLALHRSYLTPGDSSSGWTAGAAAYYVGYLQPRTFLLKYNENGTITDA | |
| | | | VDCALDPLSETKCTLGSGGGGESQVRQQFSKDIEKLLNEQVNKEMQSSNLYMSMSSWCYTHSLDGA | |
| | | | GLFLFDHAAEEYEHAKKLIIFLNENNVPVQLTSISAPEHKFEGLTQIFQKAYEHEQHISESINNIVDHAIKS | |
| | | | KDHATFNFLQWYVAEQHEEEVLFKDILDKIELIGNENHGLYLADQYVKGIAKSRKSGS | |
| pCoVP805 | R-R-N-Ferritin | His8-3c-RhGB01-MW719567 | HHHHHHHHGPLEVLFQGPNVTQLCPFSDVFNRTMFPSVYAWERMSIRDCVADYAVLYNSSISFSTFE | 442 |
| | | RBD-GSGGSG-RN-Ferritin | CYGVSPTKLNDLCFSSVYADYFVVRGDDVKQIAPGKTGVIADYNYKLPDDFVGCVLAWNSRHVDNKQ | |
| | | | GFYYRLFRHGKIKPYQRDMSRELYSPSGTPCTVVGFNCYDPLAHYAFASPGIGIGYQPFRVVVLSFQL | |
| | | | LNAPATVCGPGSGGSGSSQCVNLTTRTQLPPAYTNSFTRGVYYPDKVFRSSVLHSTQDLFLPFFSNV | |
| | | | TWFHAIHVSGTNGTKRFDNPVLPFNDGVYFASTEKSNIIRGWIFGTTLDSKTQSLLIVNNATNVVIKVCE | |
| | | | FQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP806 | R-R-N-Ferritin | His8-3c-bioinformatically | HHHHHHHHGPLEVLFQGPNITNLCPFDEVFNATRFPSVYAWERKKISNCVADYSVLYNSTSFSTFKCY | 443 |
| | | derived Cluster1_RBD (SARS- | GVSPSKLNDLCFTSVYADSFVIRGDEVRQIAPGQTGVIADYNYKLPDDFTGCVIAWNTANQDSGEGGN | |
| | | related)-GSGGSG-RN-Ferritin | PNYYYRSSRKSKLKPFERDLSSDEYNVGSQPCNRMGPNCYRTLSTYDFYPTVGVGYQPYRVVVLSFE | |
| | | | LLNAPATVCGPGSGGSGSSQCVNLTTRTQLPPAYTNSFTRGVYYPDKVFRSSVLHSTQDLFLPFFSN | |
| | | | VTWFHAIHVSGTNGTKRFDNPVLPFNDGVYFASTEKSNIIRGWIFGTTLDSKTQSLLIVNNATNVVIKVC | |
| | | | EFQFCNDPFLGVYYHKNNKSWMESEFRVYSSANNCTFEYVSQPFLMDLEGKQGNFKNLREFVFKNID | |
| | | | GYFKIYSKHTPINLVRDLPQGFSALEPLVDLPIGINITRFQTLLALHRSYLTPGDSSSGWTAGAAAYYVG | |
| | | | YLQPRTFLLKYNENGTITDAVDCALDPLSETKCTLGSGGGGESQVRQQFSKDIEKLLNEQVNKEMQSS | |
| | | | NLYMSMSSWCYTHSLDGAGLFLFDHAAEEYEHAKKLIIFLNENNVPVQLTSISAPEHKFEGLTQIFQKA | |
| | | | YEHEQHISESINNIVDHAIKSKDHATFNFLQWYVAEQHEEEVLFKDILDKIELIGNENHGLYLADQYVKGI | |
| | | | AKSRKSGS | |
| pCoVP807 | R-R-N-Ferritin | His8-3c-bioinformatically | HHHHHHHHGPLEVLFQGPNLPDCNIEEWLTAKSVPSPLNWERKTFSNCNFNLSSLLRFVQADSFSCN | 444 |
| | | derived Cluster2_RBD (OC43- | NIDASKVYGMCFGSISVDKFAIPNSRQVDLQLGNSGFLQSFNYKIDTTATSCQLYYSLPANNVTINNYN | |
| | | related)-GSGGSG-RN-Ferritin | PSSWNRRYGFNDDTVFKPRPAGVLTNHDVVYSQQCFKVPNTFCPCKNNGSCVSSGKGTTNGSGTC | |
| | | | PAGTNYRTCENTTWCACSCTPDPITASGPRKCPQKKSLVGVGEHCSGLGVDSDYCGNVSCLCSTDA | |
| | | | FLGWSADSCLVNDRCNIFANIILNGINSGTTCSTGSGGSGSSQCVNLTTRTQLPPAYTNSFTRGVYYP | |
| | | | DKVFRSSVLHSTQDLFLPFFSNVTWFHAIHVSGTNGTKRFDNPVLPFNDGVYFASTEKSNIIRGWIFGT | |
| | | | TLDSKTQSLLIVNNATNVVIKVCEFQFCNDPFLGVYYHKNNKSWMESEFRVYSSANNCTFEYVSQPFL | |
| | | | MDLEGKQGNFKNLREFVFKNIDGYFKIYSKHTPINLVRDLPQGFSALEPLVDLPIGINITRFQTLLALHRS | |
| | | | YLTPGDSSSGWTAGAAAYYVGYLQPRTFLLKYNENGTITDAVDCALDPLSETKCTLGSGGGGESQVR | |
| | | | QQFSKDIEKLLNEQVNKEMQSSNLYMSMSSWCYTHSLDGAGLFLFDHAAEEYEHAKKLIIFLNENNVP | |
| | | | VQLTSISAPEHKFEGLTQIFQKAYEHEQHISESINNIVDHAIKSKDHATFNFLQWYVAEQHEEEVLFKDIL | |
| | | | DKIELIGNENHGLYLADQYVKGIAKSRKSGS | |
| pCoVP808 | R-R-N-Ferritin | His8-3c-bioinformatically | HHHHHHHHGPLEVLFQGPQGVECDFSKLFKAAAPQIYNFSRLVFTNCNYNLTKLLSLFQVDEFSCHQI | 445 |
| | | derived Cluster3_RBD (MERS- | SPSALASGCYSSLTVDYFAYPLSMKSYLQIGSAGEISQFNYKQDFSNPTCRILASVPANVSITKPDKYIYI | |
| | | related)-GSGGSG-RN-Ferritin | SQCYSLSAYSDVDVPHYVNPGQYTSIPCLYLTSSGFDEDGQYFRYNLTQFEGQNYLVATGVTVSMTD | |
| | | | NLQMVFVITVQYGTDTNSVCPGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLGSGGGGESQVRQQFSKDIEKLLN | |
| | | | EQVNKEMQSSNLYMSMSSWCYTHSLDGAGLFLFDHAAEEYEHAKKLIIFLNENNVPVQLTSISAPEHK | |
| | | | FEGLTQIFQKAYEHEQHISESINNIVDHAIKSKDHATFNFLQWYVAEQHEEEVLFKDILDKIELIGNENHG | |
| | | | LYLADQYVKGIAKSRKSGS | |
| pCoVP809 | R-R-N-Ferritin | His8-3c-MER1 (ErinaceusCoV/ | HHHHHHHHGPLEVLFQGPELTECDLDVLFKNDAPIIANYSRRVFTNCNYNLTKLLSLVQVDEFVCHKTT | 446 |
| | | 2012-174/GER/2012) RBD- | PEALATGCYSSLTVDWFALPFSMKSTLAIGSAEAISMFNYNQDYSNPTCRIHAAVTANVSTALNFTANA | |
| | | GSGGSG-RN-Ferritin | NYAYISRCQGVDGKPILLQPGQMTNIACRSGVLARPSDADYFGYSFQGRNYYLGRKSYKPKTDEGDV | |
| | | | QMVYVITPKYDKGPDTVCPLKGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLGSGGGGESQVRQQFSKDIEKLLN | |
| | | | EQVNKEMQSSNLYMSMSSWCYTHSLDGAGLFLFDHAAEEYEHAKKLIIFLNENNVPVQLTSISAPEHK | |
| | | | FEGLTQIFQKAYEHEQHISESINNIVDHAIKSKDHATFNFLQWYVAEQHEEEVLFKDILDKIELIGNENHG | |
| | | | LYLADQYVKGIAKSRKSGS | |
| pCoVP810 | R-R-N-Ferritin | His8-3c-MER2 (Neoromicia/ | HHHHHHHHGPLEVLFQGPVYPDCNFTELFRERAPTIMQYKREVFTRCNYNLSLLLSLVQVDEFVCDKI | 447 |
| | | 5038) RBD-GSGGSG-RN- | TPEALATGCYSSLTVDWFAFPYAWKSYLAIGSADRIVRFNYNQDYSNPSCRIHSKVNSSIGISYAGAYS | |
| | | Ferritin | YITNCNYGATNKDDVVKPGGRASQQCITGALNSPTTGQLWAYNFGGVPYRVSRLTYTDHLSDPLDMV | |
| | | | YVITVKYEPGAETVCPKQGSGGSGSSQCVNLTTRTQLPPAYTNSFTRGVYYPDKVFRSSVLHSTQDL | |
| | | | FLPFFSNVTWFHAIHVSGTNGTKRFDNPVLPFNDGVYFASTEKSNIIRGWIFGTTLDSKTQSLLIVNNAT | |
| | | | NVVIKVCEFQFCNDPFLGVYYHKNNKSWMESEFRVYSSANNCTFEYVSQPFLMDLEGKQGNFKNLR | |
| | | | EFVFKNIDGYFKIYSKHTPINLVRDLPQGFSALEPLVDLPIGINITRFQTLLALHRSYLTPGDSSSGWTAG | |
| | | | AAAYYVGYLQPRTFLLKYNENGTITDAVDCALDPLSETKCTLGSGGGGESQVRQQFSKDIEKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| pCoVP811 | R-R-N-Ferritin | His8-3c-MER3 (HKU4 SM3A) | HHHHHHHHGPLEVLFQGPQATECDFSPMLTGVAPQVYNFKRLVFSNCNYNLTKLLSLFAVDEFSCNGI | 448 |
| | | RBD-GSGGSG-RN-Ferritin | SPDAIARGCYSTLTVDYFAYPLSMKSYIRPGSAGNIPLYNYKQSFANPTCRVMASVPANVTITKPDAYG | |
| | | | YISKCSRLTGANQDVETPLYINPGEYSICRDFSPGGFSEDGQVFKRTLTQFEGGGLLIGVGTRVPMTD | |
| | | | NLQMSFIIFVQYGTGTDSVCPMLGSGGSGSSQCVNLTTRTQLPPAYTNSFTRGVYYPDKVFRSSVLH | |
| | | | STQDLFLPFFSNVTWFHAIHVSGTNGTKRFDNPVLPFNDGVYFASTEKSNIIRGWIFGTTLDSKTQSLLI | |
| | | | VNNATNVVIKVCEFQFCNDPFLGVYYHKNNKSWMESEFRVYSSANNCTFEYVSQPFLMDLEGKQGN | |
| | | | FKNLREFVFKNIDGYFKIYSKHTPINLVRDLPQGFSALEPLVDLPIGINITRFQTLLALHRSYLTPGDSSSG | |
| | | | WTAGAAAYYVGYLQPRTFLLKYNENGTITDAVDCALDPLSETKCTLGSGGGGESQVRQQFSKDIEKLL | |
| | | | NEQVNKEMQSSNLYMSMSSWCYTHSLDGAGLFLFDHAAEEYEHAKKLIIFLNENNVPVQLTSISAPEH | |
| | | | KFEGLTQIFQKAYEHEQHISESINNIVDHAIKSKDHATFNFLQWYVAEQHEEEVLFKDILDKIELIGNENH | |
| | | | GLYLADQYVKGIAKSRKSGS | |
| pCoVP812 | R-R-N-Ferritin | His8-3c-MER4 (BatCoV-Ita2 | HHHHHHHHGPLEVLFQGPQGVECDFSKLFKAAPPQIYNFSRLVFTNCNYNLTKLLSLFHVSEFSCHQV | 449 |
| | | 206645-63) RBD-GSGGSG- | SPSALASGCYSSLTVDYFAYPLYLASYLQQGSTGEIAQYNYKQDFSNPTCRILASVPANVSIPKPDKYI | |
| | | RN-Ferritin | WLSQCYSFSAYSGDIPHYVQPGQYTPCLYLTSSGFDKSYQTNRDMQNKMAATGVTSSMTDNLQMAF | |
| | | | VISVQYGTDTNSVCPMGSGGSGSSQCVNLTTRTQLPPAYTNSFTRGVYYPDKVFRSSVLHSTQDLFL | |
| | | | PFFSNVTWFHAIHVSGTNGTKRFDNPVLPFNDGVYFASTEKSNIIRGWIFGTTLDSKTQSLLIVNNATN | |
| | | | WIKVCEFQFCNDPFLGVYYHKNNKSWMESEFRVYSSANNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLGSGGGGESQVRQQFSKDIEKLLNEQVNK | |
| | | | EMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoVP813 | R-R-N-Ferritin | His8-3c-MER5 (BatCoV-Ita1 | HHHHHHHHGPLEVLFQGPQGVECDFSKLFKAAPPQIYNFSRLVFTNCNYNLTKLLSLFHVSEFSCHQV | 450 |
| | | 206645-40) RBD-GSGGSG- | SPSALASGCYSSLTVDYFAYPLYLASYLQQGSTGEIAQYNYKQDFSNPTCRILASVPANVSIPKPDKYI | |
| | | RN-Ferritin | WLSQCYSFSAYSGDVPHYVLPGQYTPCLYLTSSGFDNSYQTNRDFQNKMAATGVISSMTDNLQMAF | |
| | | | VISVQYGTDTNSVCPMGSGGSGSSQCVNLTTRTQLPPAYTNSFTRGVYYPDKVFRSSVLHSTQDLFL | |
| | | | PFFSNVTWFHAIHVSGTNGTKRFDNPVLPFNDGVYFASTEKSNIIRGWIFGTTLDSKTQSLLIVNNATN | |
| | | | WIKVCEFQFCNDPFLGVYYHKNNKSWMESEFRVYSSANNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAA | |
| | | | AYYVGYLQPRTFLLKYNENGTITDAVDCALDPLSETKCTLGSGGGGESQVRQQFSKDIEKLLNEQVNK | |
| | | | EMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| pCoVP814 | R-R-N-Ferritin | His8-3c-HKU5 RBD-GSGGSG- | HHHHHHHHGPLEVLFQGPTTQECDFTPMLTGTPPPIYNFKRLVFTNCNYNLTKLLSLFQVSEFSCHQV | 451 |
| | | RN-Ferritin | SPSSLATGCYSSLTVDYFAYSTDMSSYLQPGSAGAIVQFNYKQDFSNPTCRVLATVPQNLTTITKPSN | |
| | | | YAYLTECYKTSAYGKNYLYNAPGAYTPCLSLASRGFSTKYQSHSDGELTTTGYIYPVTGNLQMAFIISV | |
| | | | QYGTDTNSVCPMGSGGSGSSQCVNLTTRTQLPPAYTNSFTRGVYYPDKVFRSSVLHSTQDLFLPFFS | |
| | | | NVTWFHAIHVSGTNGTKRFDNPVLPFNDGVYFASTEKSNIIRGWIFGTTLDSKTQSLLIVNNATNVVIKV | |
| | | | CEFQFCNDPFLGVYYHKNNKSWMESEFRVYSSANNCTFEYVSQPFLMDLEGKQGNFKNLREFVFKNI | |
| | | | DGYFKIYSKHTPINLVRDLPQGFSALEPLVDLPIGINITRFQTLLALHRSYLTPGDSSSGWTAGAAAYYV | |
| | | | GYLQPRTFLLKYNENGTITDAVDCALDPLSETKCTLGSGGGGESQVRQQFSKDIEKLLNEQVNKEMQ | |
| | | | SSNLYMSMSSWCYTHSLDGAGLFLFDHAAEEYEHAKKLIIFLNENNVPVQLTSISAPEHKFEGLTQIFQ | |
| | | | KAYEHEQHISESINNIVDHAIKSKDHATFNFLQWYVAEQHEEEVLFKDILDKIELIGNENHGLYLADQYV | |
| | | | KGIAKSRKSGS | |
| pCoVP815 | R-R-N-Ferritin | His8-3c-MERS-Eng1 RBD- | HHHHHHHHGPLEVLFQGPEGVECDFSPLLSGTPPQVYNFKRLVFTNCNYNLTKLLSLFSVNDFTCSQI | 452 |
| | | GSGGSG-RN-Ferritin | SPAAIASNCYSSLILDYFSYPLSMKSDLSVSSAGPISQFNYKQSFSNPTCLILATVPHNLTTITKPLKYSYI | |
| | | | NKCSRFLSDDRTEVPQLVNANQYSPCVSIVPSTVWEDGDYYRKQLSPLEGGGWLVASGSTVAMTEQ | |
| | | | LQMGFGITVQYGTDTNSVCPKGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLGSGGGGESQVRQQFSKDIEKLLN | |
| | | | EQVNKEMQSSNLYMSMSSWCYTHSLDGAGLFLFDHAAEEYEHAKKLIIFLNENNVPVQLTSISAPEHK | |
| | | | FEGLTQIFQKAYEHEQHISESINNIVDHAIKSKDHATFNFLQWYVAEQHEEEVLFKDILDKIELIGNENHG | |
| | | | LYLADQYVKGIAKSRKSGS | |
| pCoVP816 | R-R-N-Ferritin | His8-3c-MERS-EMC RBD- | HHHHHHHHGPLEVLFQGPEGVECDFSPLLSGTPPQVYNFKRLVFTNCNYNLTKLLSLFSVNDFTCSQI | 453 |
| | | GSGGSG-RN-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQYGTDTNSVCPKGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLGSGGGGESQVRQQFSKDIEKLLN | |
| | | | EQVNKEMQSSNLYMSMSSWCYTHSLDGAGLFLFDHAAEEYEHAKKLIIFLNENNVPVQLTSISAPEHK | |
| | | | FEGLTQIFQKAYEHEQHISESINNIVDHAIKSKDHATFNFLQWYVAEQHEEEVLFKDILDKIELIGNENHG | |
| | | | LYLADQYVKGIAKSRKSGS | |
| pCoVP817 | RBD-Ferritin | His8-3c-Clu1-RBD-Ferritin | HHHHHHHHGPLEVLFQGPNITNLCPFDEVFNATRFPSVYAWERKKISNCVADYSVLYNSTSFSTFKCY | 454 |
| | | | GVSPSKLNDLCFTSVYADSFVIRGDEVRQIAPGQTGVIADYNYKLPDDFTGCVIAWNTANQDSGEGGN | |
| | | | PNYYYRSSRKSKLKPFERDLSSDEYNVGSQPCNRMGPNCYRTLSTYDFYPTVGVGYQPYRVVVLSFE | |
| | | | LLNAPATVCGP | |
| | | | GSGGGGESQVRQQFSKDIEKLLNEQVNKEMQSSNLYMSMSSWCYTHSLDGAGLFLFDHAAEEYEHA | |
| | | | KKLIIFLNENNVPVQLTSISAPEHKFEGLTQIFQKAYEHEQHISESINNIVDHAIKSKDHATFNFLQWYVA | |
| | | | EQHEEEVLFKDILDKIELIGNENHGLYLADQYVKGIAKSRKSGS | |
| pCoVP818 | RBD-Ferritin | His8-3c-Clu1-RBD(131_ | HHHHHHHHGPLEVLFQGPNITNLCPFDEVFNATRFPSVYAWERKKISNCVADYSVLYNSTSFSTFKCY | 455 |
| | | like)-Ferritin | GVSPSKLNDLCFTSVYADSFVIRGDEVRQIAPGQTGVIADYNYKLPDDFTGCVIAWNTANQDSGEGGN | |
| | | | PNYYRRSSRKSKLKPFERDLSSDEYNVGSQPCNRMGPNCYRTLSTYDFYPTVGVGYQPYRVVVLSFE | |
| | | | NKSAPATVCGP | |
| | | | GSGGGGESQVRQQFSKDIEKLLNEQVNKEMQSSNLYMSMSSWCYTHSLDGAGLFLFDHAAEEYEHA | |
| | | | KKLIIFLNENNVPVQLTSISAPEHKFEGLTQIFQKAYEHEQHISESINNIVDHAIKSKDHATFNFLQWYVA | |
| | | | EQHEEEVLFKDILDKIELIGNENHGLYLADQYVKGIAKSRKSGS | |
| pCoVP819 | RBD-RBD-Ferritin | His8-3c-Clu1-RBD-GSGGSG- | HHHHHHHHGPLEVLFQGPNITNLCPFDEVFNATRFPSVYAWERKKISNCVADYSVLYNSTSFSTFKCY | 456 |
| | | M3RBD-Ferritin | GVSPSKLNDLCFTSVYADSFVIRGDEVRQIAPGQTGVIADYNYKLPDDFTGCVIAWNTANQDSGEGGN | |
| | | | PNYYYRSSRKSKLKPFERDLSSDEYNVGSQPCNRMGPNCYRTLSTYDFYPTVGVGYQPYRVVVLSFE | |
| | | | LLNAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVWEDGDYYRKQLSPLEGGGWLVASGSTVAMTEQL | |
| | | | QMGFGITVQNGTDTNSVCPKGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoVP820 | RBD-RBD-Ferritin | His8-3c-Clu1-RBD(131_ | HHHHHHHHGPLEVLFQGPNITNLCPFDEVFNATRFPSVYAWERKKISNCVADYSVLYNSTSFSTFKCY | 457 |
| | | like)-GSGGSG-M3RBD-Ferritin | GVSPSKLNDLCFTSVYADSFVIRGDEVRQIAPGQTGVIADYNYKLPDDFTGCVIAWNTANQDSGEGGN | |
| | | | PNYYRRSSRKSKLKPFERDLSSDEYNVGSQPCNRMGPNCYRTLSTYDFYPTVGVGYQPYRVVVLSFE | |
| | | | NKSAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVWEDGDYYRKQLSPLEGGGWLVASGSTVAMTEQL | |
| | | | QMGFGITVQNGTDTNSVCPKGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoVP821 | RBD-RBD-Ferritin | His8-3c-M3RBD-GSGGSG- | HHHHHHHHGPLEVLFQGPEGVECDFSPLLSGTPPQVYNFKRLVFTNCNYNLTKLLSLFSVNDFTCSQI | 458 |
| | | Clu1-RBD-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGSGNITNLCPFDEVFNATRFPSVYAWERKKISNCVADYSVLYN | |
| | | | STSFSTFKCYGVSPSKLNDLCFTSVYADSFVIRGDEVRQIAPGQTGVIADYNYKLPDDFTGCVIAWNTA | |
| | | | NQDSGEGGNPNYYYRSSRKSKLKPFERDLSSDEYNVGSQPCNRMGPNCYRTLSTYDFYPTVGVGY | |
| | | | QPYRVVVLSFELLNAPATVCGPGSGGGGESQVRQQFSKDIEKLLNEQVNKEMQSSNLYMSMSSWCY | |
| | | | THSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| pCoVP822 | RBD-RBD-Ferritin | His8-3c-M3RBD-GSGGSG-Clu1- | HHHHHHHHGPLEVLFQGPEGVECDFSPLLSGTPPQVYNFKRLVFTNCNYNLTKLLSLFSVNDFTCSQI | 459 |
| | | RBD(131_like)-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVWEDGDYYRKQLSPLEGGGWLVASGSTVAMTEQ | |
| | | | LQMGFGITVQNGTDTNSVCPKGSGGSGNITNLCPFDEVFNATRFPSVYAWERKKISNCVADYSVLYN | |
| | | | STSFSTFKCYGVSPSKLNDLCFTSVYADSFVIRGDEVRQIAPGQTGVIADYNYKLPDDFTGCVIAWNTA | |
| | | | NQDSGEGGNPNYYRRSSRKSKLKPFERDLSSDEYNVGSQPCNRMGPNCYRTLSTYDFYPTVGVGY | |
| | | | QPYRWVVLSFENKSAPATVCGPGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pCoVP823 | RBD-RBD-Ferritin | His8-3c-Clu1-RBD-GSGGSG- | HHHHHHHHGPLEVLFQGPNITNLCPFDEVFNATRFPSVYAWERKKISNCVADYSVLYNSTSFSTFKCY | 460 |
| | | M3.7RBD-Ferritin | GVSPSKLNDLCFTSVYADSFVIRGDEVRQIAPGQTGVIADYNYKLPDDFTGCVIAWNTANQDSGEGGN | |
| | | | PNYYYRSSRKSKLKPFERDLSSDEYNVGSQPCNRMGPNCYRTLSTYDFYPTVGVGYQPYRVVVLSFE | |
| | | | LLNAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVAEDGDYYRKQLSPLEGGGWLVASGSTVAMTEQLQ | |
| | | | MGFGITVQNGTDTNSVCPKGSGGGGESQVRQQFSKDIEKLLNEQVNKEMQSSNLYMSMSSWCYTH | |
| | | | SLDGAGLFLFDHAAEEYEHAKKLIIFLNENNVPVQLTSISAPEHKFEGLTQIFQKAYEHEQHISESINNIV | |
| | | | DHAIKSKDHATFNFLQWYVAEQHEEEVLFKDILDKIELIGNENHGLYLADQYVKGIAKSRKSGS | |
| pCoVP824 | RBD-RBD-Ferritin | His8-3c-Clu1-RBD(131_ | HHHHHHHHGPLEVLFQGPNITNLCPFDEVFNATRFPSVYAWERKKISNCVADYSVLYNSTSFSTFKCY | 461 |
| | | like)-GSGGSG-M3.7RBD- | GVSPSKLNDLCFTSVYADSFVIRGDEVRQIAPGQTGVIADYNYKLPDDFTGCVIAWNTANQDSGEGGN | |
| | | Ferritin | PNYYRRSSRKSKLKPFERDLSSDEYNVGSQPCNRMGPNCYRTLSTYDFYPTVGVGYQPYRVVVLSFE | |
| | | | NKSAPATVCGPGSGGSGEGVECDFSPLLSGTPPQVYNFKRLVFTNCNYNLTKLLSLFSVNDFTCSQIS | |
| | | | PAAIASNCYSSLILDYFSYPLSMKSDLSVSSAGPISQFNYKQSFSNPTCLILATVPHNLTTITKPLKYSYIN | |
| | | | KCSRLLSDDRTEVPQLVNANQYSPCVSIVPSTVAEDGDYYRKQLSPLEGGGWLVASGSTVAMTEQLQ | |
| | | | MGFGITVQNGTDTNSVCPKGSGGGGESQVRQQFSKDIEKLLNEQVNKEMQSSNLYMSMSSWCYTH | |
| | | | SLDGAGLFLFDHAAEEYEHAKKLIIFLNENNVPVQLTSISAPEHKFEGLTQIFQKAYEHEQHISESINNIV | |
| | | | DHAIKSKDHATFNFLQWYVAEQHEEEVLFKDILDKIELIGNENHGLYLADQYVKGIAKSRKSGS | |
| pCoVP825 | RBD-RBD-Ferritin | His8-3c-M3.7RBD-GSGGSG- | HHHHHHHHGPLEVLFQGPEGVECDFSPLLSGTPPQVYNFKRLVFTNCNYNLTKLLSLFSVNDFTCSQI | 462 |
| | | Clu1-RBD-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVAEDGDYYRKQLSPLEGGGWLVASGSTVAMTEQL | |
| | | | QMGFGITVQNGTDTNSVCPKGSGGSGNITNLCPFDEVFNATRFPSVYAWERKKISNCVADYSVLYNS | |
| | | | TSFSTFKCYGVSPSKLNDLCFTSVYADSFVIRGDEVRQIAPGQTGVIADYNYKLPDDFTGCVIAWNTAN | |
| | | | QDSGEGGNPNYYYRSSRKSKLKPFERDLSSDEYNVGSQPCNRMGPNCYRTLSTYDFYPTVGVGYQP | |
| | | | YRVVVLSFELLNAPATVCGPGSGGGGESQVRQQFSKDIEKLLNEQVNKEMQSSNLYMSMSSWCYTH | |
| | | | SLDGAGLFLFDHAAEEYEHAKKLIIFLNENNVPVQLTSISAPEHKFEGLTQIFQKAYEHEQHISESINNIV | |
| | | | DHAIKSKDHATFNFLQWYVAEQHEEEVLFKDILDKIELIGNENHGLYLADQYVKGIAKSRKSGS | |
| pCoVP826 | RBD-RBD-Ferritin | His8-3c-M3.7RBD-GSGGSG- | HHHHHHHHGPLEVLFQGPEGVECDFSPLLSGTPPQVYNFKRLVFTNCNYNLTKLLSLFSVNDFTCSQI | 463 |
| | | Clu1-RBD(131_like)-Ferritin | SPAAIASNCYSSLILDYFSYPLSMKSDLSVSSAGPISQFNYKQSFSNPTCLILATVPHNLTTITKPLKYSYI | |
| | | | NKCSRLLSDDRTEVPQLVNANQYSPCVSIVPSTVAEDGDYYRKQLSPLEGGGWLVASGSTVAMTEQL | |
| | | | QMGFGITVQNGTDTNSVCPKGSGGSGNITNLCPFDEVFNATRFPSVYAWERKKISNCVADYSVLYNS | |
| | | | TSFSTFKCYGVSPSKLNDLCFTSVYADSFVIRGDEVRQIAPGQTGVIADYNYKLPDDFTGCVIAWNTAN | |
| | | | QDSGEGGNPNYYRRSSRKSKLKPFERDLSSDEYNVGSQPCNRMGPNCYRTLSTYDFYPTVGVGYQ | |
| | | | PYRVVVLSFENKSAPATVCGPGSGGGGESQVRQQFSKDIEKLLNEQVNKEMQSSNLYMSMSSWCYT | |
| | | | HSLDGAGLFLFDHAAEEYEHAKKLIIFLNENNVPVQLTSISAPEHKFEGLTQIFQKAYEHEQHISESINNI | |
| | | | VDHAIKSKDHATFNFLQWYVAEQHEEEVLFKDILDKIELIGNENHGLYLADQYVKGIAKSRKSGS | |
| pCoVP827 | RBD-Ferritin | His8-3c-Clu3-RBD-Ferritin | HHHHHHHHGPLEVLFQGPQGVECDFSKLFKAAAPQIYNFSRLVFTNCNYNLTKLLSLFQVDEFSCHQI | 464 |
| | | | SPSALASGCYSSLTVDYFAYPLSMKSYLQIGSAGEISQFNYKQDFSNPTCRILASVPANVSITKPDKYIYI | |
| | | | SQCYSLSAYSDVDVPHYVNPGQYTSIPCLYLTSSGFDEDGQYFRYNLTQFEGQNYLVATGVTVSMTD | |
| | | | NLQMVFVITVQYGTDTNSVCPGGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pCoVP828 | RBD-Ferritin | His8-3c-Clu3(M3like- | HHHHHHHHGPLEVLFQGPQGVECDFSKLFKAAAPQIYNFSRLVFTNCNYNLTKLLSLFQVDEFSCHQI | 465 |
| | | baseglycan)-RBD-Ferritin | SPSALASGCYSSLTVDYFAYPLSMKSYLQIGSAGEISQFNYKQDFSNPTCRILASVPANVSITKPDKYIYI | |
| | | | SQCYSLSAYSDVDVPHYVNPGQYTSIPCLYLTSSGFDEDGQYFRYNLTQFEGQNYLVATGVTVSMTD | |
| | | | NLQMVFVITVQNGTDTNSVCPGGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pCoVP829 | RBD-Ferritin | His8-3c-Clu3(M3.2like- | HHHHHHHHGPLEVLFQGPQGVECDFSKLFKAAAPQIYNFSRLVFTNCNYNLTKLLSLFQVDEFSCHQI | 466 |
| | | baseglycan_L506R)-RBD- | SPSALASGCYSSLTVDYFAYPLSMKSYLQIGSAGEISQFNYKQDFSNPTCRILASVPANVSITKPDKYIYI | |
| | | Ferritin | SQCYSRSAYSDVDVPHYVNPGQYTSIPCLYLTSSGFDEDGQYFRYNLTQFEGQNYLVATGVTVSMTD | |
| | | | NLQMVFVITVQNGTDTNSVCPGGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pCoVP830 | RBD-Ferritin | His8-3c-Clu3(M3.6like- | HHHHHHHHGPLEVLFQGPQGVECDFSKLFKAAAPQIYNFSRLVFTNCNYNLTKLLSLFQVDEFSCHQI | 467 |
| | | baseglycan_Y523R)-RBD- | SPSALASGCYSSLTVDYFAYPLSMKSYLQIGSAGEISQFNYKQDFSNPTCRILASVPANVSITKPDKYIYI | |
| | | Ferritin | SQCYSRSAYSDVDVPHYVNPGQRTSIPCLYLTSSGFDEDGQYFRYNLTQFEGQNYLVATGVTVSMTD | |
| | | | NLQMVFVITVQNGTDTNSVCPGGSGGGGESQVRQQFSKDIEKLLNEQVNKEMQSSNLYMSMSSWC | |
| | | | YTHSLDGAGLFLFDHAAEEYEHAKKLIIFLNENNVPVQLTSISAPEHKFEGLTQIFQKAYEHEQHISESIN | |
| | | | NIVDHAIKSKDHATFNFLQWYVAEQHEEEVLFKDILDKIELIGNENHGLYLADQYVKGIAKSRKSGS | |
| pCoVM01 | MERS SpFN | MERS England-1 SpFN | YVDVGPDSVKSACIEVDIQQTFFDKTWPRPIDVSKADGIIYPQGRTYSNITITYQGLFPYQGDHGDMYV | 468 |
| | | (1b06vOnogly) | YSAGHATGTTPQKLFVANYSQDVKQFANGFWVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD468 | |
| | | | QLNSSYFKLSIPTNFSFGVTQEYIQTTIQKVTVDCKQYVQNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSTGIDFQDELDEIHKQGSGGSGDIIKLL | |
| | | | NEQVNKEMQSSNLYMSMSSWCYTHSLDGAGLFLFDHAAEEYEHAKKLIIFLNENNVPVQLTSISAPEH | |
| | | | KFEGLTQIFQKAYEHEQHISESINNIVDHAIKSKDHATFNFLQWYVAEQHEEEVLFKDILDKIELIGNENH | |
| | | | GLYLADQYVKGIAKSRKSGS | |
| pCoVM02 | MERS SpFN | MERS | YVDVGPDSVKSACIEVDIQQTFFDKTWPRPIDVSKADGIIYPQGRTYSNITITYQGLFPYQGDHGDMYV | 469 |
| | | England-1 SpFN (1b06v0) | YSAGHATGTTPQKLFVANYSQDVKQFANGFVVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD469 | |
| | | | QLNSSYFKLSIPTNFSFGVTQEYIQTTIQKVTVDCKQYVCNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSTGIDFQDELDEIHKNGSGGSGDIIKLL | |
| | | | NEQVNKEMQSSNLYMSMSSWCYTHSLDGAGLFLFDHAAEEYEHAKKLIIFLNENNVPVQLTSISAPEH | |
| | | | KFEGLTQIFQKAYEHEQHISESINNIVDHAIKSKDHATFNFLQWYVAEQHEEEVLFKDILDKIELIGNENH | |
| | | | GLYLADQYVKGIAKSRKSGS | |
| pCoVM03 | MERS SpFN | MERS England-1 SpFN | YVDVGPDSVKSACIEVDIQQTFFDKTWPRPIDVSKADGIIYPQGRTYSNITITYQGLFPYQGDHGDMYV | 470 |
| | | (1b06v1nogly) | YSAGHATGTTPQKLFVANYSQDVKQFANGFVVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD | |
| | | | QLNSSYFKLSIPTNFSFGVTQEYIQTTIQKVTVDCKQYVCNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSELQDIQDELDEIHKQGSGGSGDIIKLL | |
| | | | NEQVNKEMQSSNLYMSMSSWCYTHSLDGAGLFLFDHAAEEYEHAKKLIIFLNENNVPVQLTSISAPEH | |
| | | | KFEGLTQIFQKAYEHEQHISESINNIVDHAIKSKDHATFNFLQWYVAEQHEEEVLFKDILDKIELIGNENH | |
| | | | GLYLADQYVKGIAKSRKSGS | |
| pCoVM04 | MERS SpFN | MERS England-1 SpFN | YVDVGPDSVKSACIEVDIQQTFFDKTWPRPIDVSKADGIIYPQGRTYSNITITYQGLFPYQGDHGDMYV | 471 |
| | | (1b06v1) | YSAGHATGTTPQKLFVANYSQDVKQFANGFVVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD | |
| | | | QLNSSYFKLSIPTNFSFGVTQEYIQTTIQKVTVDCKQYVCNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSELQDIQDELDEIHKNGSGGSGDIIKLL | |
| | | | NEQVNKEMQSSNLYMSMSSWCYTHSLDGAGLFLFDHAAEEYEHAKKLIIFLNENNVPVQLTSISAPEH | |
| | | | KFEGLTQIFQKAYEHEQHISESINNIVDHAIKSKDHATFNFLQWYVAEQHEEEVLFKDILDKIELIGNENH | |
| | | | GLYLADQYVKGIAKSRKSGS | |
| pCoVM05 | MERS SpFN | MERS England-1 SpFN | YVDVGPDSVKSACIEVDIQQTFFDKTWPRPIDVSKADGIIYPQGRTYSNITITYQGLFPYQGDHGDMYV | 472 |
| | | (1b06v2nogly) | YSAGHATGTTPQKLFVANYSQDVKQFANGFVVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD | |
| | | | QLNSSYFKLSIPTNFSFGVTQEYIQTTIQKVTVDCKQYVCNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSELQDLQDELDELHKQGSGGSGDIIKL | |
| | | | LNEQVNKEMQSSNLYMSMSSWCYTHSLDGAGLFLFDHAAEEYEHAKKLIIFLNENNVPVQLTSISAPE | |
| | | | HKFEGLTQIFQKAYEHEQHISESINNIVDHAIKSKDHATFNFLQWYVAEQHEEEVLFKDILDKIELIGNEN | |
| | | | HGLYLADQYVKGIAKSRKSGS | |
| pCoVM06 | MERS SpFN | MERS England-1 SpFN | YVDVGPDSVKSACIEVDIQQTFFDKTWPRPIDVSKADGIIYPQGRTYSNITITYQGLFPYQGDHGDMYV | 473 |
| | | (1b06v2) | YSAGHATGTTPQKLFVANYSQDVKQFANGFVVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD | |
| | | | QLNSSYFKLSIPTNFSFGVTQEYIQTTIQKVTVDCKQYVCNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSELQDLQDELDELHKNGSGGSGDIIKL | |
| | | | LNEQVNKEMQSSNLYMSMSSWCYTHSLDGAGLFLFDHAAEEYEHAKKLIIFLNENNVPVQLTSISAPE | |
| | | | HKFEGLTQIFQKAYEHEQHISESINNIVDHAIKSKDHATFNFLQWYVAEQHEEEVLFKDILDKIELIGNEN | |
| | | | HGLYLADQYVKGIAKSRKSGS | |
| pCoVM07 | MERS SpFN | MERS England-1 SpFN | YVDVGPDSVKSACIEVDIQQTFFDKTWPRPIDVSKADGIIYPQGRTYSNITITYQGLFPYQGDHGDMYV | 474 |
| | | (1b06v3nogly) | YSAGHATGTTPQKLFVANYSQDVKQFANGFVVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD | |
| | | | QLNSSYFKLSIPTNFSFGVTQEYIQTTIQKVTVDCKQYVQNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSTLQDIQDELDEIHKQGSGGSGDIIKLL | |
| | | | NEQVNKEMQSSNLYMSMSSWCYTHSLDGAGLFLFDHAAEEYEHAKKLIIFLNENNVPVQLTSISAPEH | |
| | | | KFEGLTQIFQKAYEHEQHISESINNIVDHAIKSKDHATFNFLQWYVAEQHEEEVLFKDILDKIELIGNENH | |
| | | | GLYLADQYVKGIAKSRKSGS | |
| pCoVM08 | MERS SpFN | MERS England-1 SpFN | YVDVGPDSVKSACIEVDIQQTFFDKTWPRPIDVSKADGIIYPQGRTYSNITITYQGLFPYQGDHGDMYV | 475 |
| | | (1b06v4nogly) | YSAGHATGTTPQKLFVANYSQDVKQFANGFVVRIGAAANSTGTVIISPSTSATIRKIYPAFMLGSSVGN | |
| | | | FSDGKMGRFFNHTLVLLPDGCGTLLRAFYCILEPRSGNHCPAGNSYTSFATYHTPATDCSDGNYNRN | |
| | | | ASLNSFKEYFNLRNCTFMYTYNITEDEILEWFGITQTAQGVHLFSSRYVDLYGGNMFQFATLPVYDTIK | |
| | | | YYSIIPHSIRSIQSDRKAWAAFYVYKLQPLTFLLDFSVDGYIRRAIDCGFNDLSQLHCSYESFDVESGVY | |
| | | | SVSSFEAKPSGSVVEQAEGVECDFSPLLSGTPPQVYNFKRLVFTNCNYNLTKLLSLFSVNDFTCSQISP | |
| | | | AAIASNCYSSLILDYFSYPLSMKSDLSVSSAGPISQFNYKQSFSNPTCLILATVPHNLTTITKPLKYSYINK | |
| | | | CSRFLSDDRTEVPQLVNANQYSPCVSIVPSTVWEDGDYYRKQLSPLEGGGWLVASGSTVAMTEQLQ | |
| | | | MGFGITVQYGTDTNSVCPKLEFANDTKIASQLGNCVEYSLYGVSGRGVFQNCTAVGVRQQRFVYDAY | |
| | | | QNLVGYYSDDGNYYCLRACVSVPVSVIYDKETKTHATLFGSVACEHISSTMSQYSRSTRSMLKRRDST | |
| | | | YGPLQTPVGCVLGLVNSSLFVEDCKLPLGQSLCALPDTPSTLTPASVGSVPGEMRLASIAFNHPIQVD | |
| | | | QLNSSYFKLSIPTNFSFGVTQEYIQTTIQKVTVDCKQYVQNGFQKCEQLLREYGQFCSKINQALHGANL | |
| | | | RQDDSVRNLFASVKSSQSSPIIPGFGGDFNLTLLEPVSISTGSRSARSAIEDLLFDKVTIADPGYMQGY | |
| | | | DDCMQQGPASARDLICAQYVAGYKVLPPLMDVNMEAAYTSSLLGSIAGVGWTAGLSSFAAIPFAQSIF | |
| | | | YRLNGVGITQQVLSENQKLIANKFNQALGAMQTGFTTTNEAFHKVQDAVNNNAQALSKLASELSNTFG | |
| | | | AISASIGDIIQRLDPPEQDAQIDRLINGRLTTLNAFVAQQLVRSESAALSAQLAKDKVNECVKAQSKRSG | |
| | | | FCGQGTHIVSFVVNAPNGLYFMHVGYYPSNHIEVVSAYGLCDAANPTNCIAPVNGYFIKTNNTRIVDE | |
| | | | WSYTGSSFYAPEPITSLNTKYVAPQVTYQNISTNLPPPLLGNSTLQDLQDELDELHKQGSGGSGDIIKL | |
| | | | LNEQVNKEMQSSNLYMSMSSWCYTHSLDGAGLFLFDHAAEEYEHAKKLIIFLNENNVPVQLTSISAPE | |
| | | | HKFEGLTQIFQKAYEHEQHISESINNIVDHAIKSKDHATFNFLQWYVAEQHEEEVLFKDILDKIELIGNEN | |
| | | | HGLYLADQYVKGIAKSRKSGS | |
| | RFN | Beta RFN | MDSKGSSQKGSRLLLLLVVSNLLLPQGVVGQNITNLCPFGEVFNATRFASVYAWNRKRISNCVADYSV | 476 |
| | | | LYNSASFSTFKCYGVSPTKLNDLCFTNVYADSFVIRGDEVRQIAPGQTGNIADYNYKLPDDFTGCVIAW | |
| | | | NSNNLDSKVGGNYNYLRRLFRKSNLKPFERDISTEIYQAGSTPCNGVKGFNCYFPLQSYGFQPTYGV | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | RFN | BA.4/5 RFN | MDSKGSSQKGSRLLLLLVVSNLLLPQGWVGQNITNLCPFDEVFNATRFASVYAWNRKRISNCVADYSV | 477 |
| | | | LYNFAPFFAFKCYGVSPTKLNDLCFTNVYADSFVIRGNEVSQIAPGQTGNIADYNYKLPDDFTGCVIAW | |
| | | | NSNKLDSKVGGNYNYRRRLFRKSNLKPFERDISTEIYQAGNKPCNGVAGVNCYFPLQSYGFRPTYGV | |
| | | | GHQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | RFN | Khosta-2 RFN | MDSKGSSQKGSRLLLLLVVSNLLLPQGVVGQNMTNICPFDQVFNKTQFPSVYAWERVRISDCVSDYT | 478 |
| | | | VLYNSSASFSTFKCYGVSPTKLNDLCFSGVYADYFVVKGDHVHQIAPGQTGVIADYNYKLPSEFVGCIL | |
| | | | AWNTRTIDSKRGFYYRLFRHGNIRPYERDTSNVPYNAAGGTCNQPGTHNCYEPLQDYGFTSTSGVG | |
| | | | YQPFRVVVLSFENKSAPATVCGGGSGGSGESQVRQQFSKDIEKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESI | |
| | | | NNIVDHAIKSKDHATFNFLQWYVAEQHEEEVLFKDILDKIELIGNENHGLYLADQYVKGIAKSRKSGS | |
| | RFN | BANAL20-247 RFN | MDSKGSSQKGSRLLLLLVVSNLLLPQGVVGQNITNFCPFDKVFNATRFPNVYAWQRTKISDCIADYTVL | 479 |
| | | | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGESQVRQQFSKDIEKLLNEQVNKEMQSSNLYMSMSSWCYTHSLDGAGLFLFDHAAEEY | |
| | | | EHAKKLIIFLNENNVPVQLTSISAPEHKFEGLTQIFQKAYEHEQHISESINNIVDHAIKSKDHATFNFLQW | |
| | | | YVAEQHEEEVLFKDILDKIELIGNENHGLYLADQYVKGIAKSRKSGS | |
| | SpFN | BA.4/5 SpFN | MDSKGSSQKGSRLLLLLVVSNLLLPQGWGQSSQCVNLITRTQSYTNSFTRGVYYPDKVFRSSVLHST | 480 |

| | | | | |
|---|---|---|---|---|
| | | | QDLFLPFFSNVTWFHAISGTNGTKRFDNPVLPFNDGVYFASTEKSNIIRGWIFGTTLDSKTQSLLIVNNA | |
| | | | TNVVIKVCEFQFCNDPFLDVYYHKNNKSWMESEFRVYSSANNCTFEYVSQPFLMDLEGKQGNFKNLR | |
| | | | EFVFKNIDGYFKIYSKHTPINLGRDLPQGFSALEPLVDLPIGINITRFQTLLALHRSYLTPGDSSSGWTAG | |
| | | | AAAYYVGYLQPRTFLLKYNENGTITDAVDCALDPLSETKCTLKSFTVEKGIYQTSNFRVQPTESIVRFPN | |
| | | | ITNLCPFDEVFNATRFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSKVGGNYNYRYRLFRKSNLKPFERDI | |
| | | | STEIYQAGNKPCNGVAGVNCYFPLQSYGFRPTYGVGHQPYRVVVLSFELLHAPATVCGPKKSTNLVK | |
| | | | NKCVNFNFNGLTGTGVLTESNKKFLPFQQFGRDIADTTDAVRDPQTLEILDITPCSFGGVSVITPGTNT | |
| | | | SNQVAVLYQGVNCTEVPVAIHADQLTPTWRVYSTGSNVFQTRAGCLIGAEYVNNSYECDIPIGAGICA | |
| | | | SYQTQTKSHGSASSVASQSIIAYTMSLGAENSVAYSNNSIAIPTNFTISVTTEILPVSMTKTSVDCTMYIC | |
| | | | GDSTECSNLLLQYGSFCTQLKRALTGIAVEQDKNTQEVFAQVKQIYKTPPIKYFGGFNFSQILPDPSKP | |
| | | | SKRSFIEDLLFNKVTLADAGFIKQYGDCLGDIAARDLICAQKFNGLTVLPPLLTDEMIAQYTSALLAGTIT | |
| | | | SGWTFGAGAALQIPFAMQMAYRFNGIGVTQNVLYENQKLIANQFNSAIGKIQDSLSSTASALGKLQDV | |
| | | | VNHNAQALNTLVKQLSSKFGAISSVLNDILSRLDPPEAEVQIDRLITGRLQSLQTYVTQQLIRAAEIRASA | |
| | | | NLAATKMSECVLGQSKRVDFCGKGYHLMSFPQSAPHGVVFLHVTYVPAQEKNFTTAPAICHDGKAHF | |
| | | | PREGVFVSNGTHWFVTQRNFYEPQIITTDNTFVSGNCDVVIGIVNNTVYDPLQSELDSIKEELDKIHKN | |
| | | | GSGGSGDIIKLLNEQVNKEMQSSNLYMSMSSWCYTHSLDGAGLFLFDHAAEEYEHAKKLIIFLNENNV | |
| | | | PVQLTSISAPEHKFEGLTQIFQKAYEHEQHISESINNIVDHAIKSKDHATFNFLQWYVAEQHEEEVLFKDI | |
| | | | LDKIELIGNENHGLYLADQYVKGIAKSRKSGS | |
| | SpFN | Khosta-2 SpFN | MDSKGSSQKGSRLLLLLVVSNLLLPQGVVGQSSQDGCATLSTKLTPTQLQVNSTRRGFYYFDDIYRSS | 481 |
| | | | LRVLVPGYFLPFGTNITRYQAQVINWTAIFFDNPVIPFNDGVYFAALEHSNVVRGWIFGTTLDNTTQSAI | |
| | | | WVNNATHILISVCYFNVCKEPMFAVSNFKPYKSWVYDRANNCTFNRAYPLNISTTPEPGKFKALREQVF | |
| | | | RYQDGFLYIYHSYESINSDTLPPGFSVLKPMLKLPLGLNITRFRVVMAMHSLTTSNFNTHSVNYFVGHL | |
| | | | KPLTMLVEFSPNGTIIDAIDCSQDPLSELKCTTKSFSVEKGIYQTSNFRVSPSLDVVRFPNMTNICPFDQ | |
| | | | VFNKTQFPSVYAWERVRISDCVSDYTVLYNSSASFSTFKCYGVSPTKLNDLCFSGVYADYFVVKGDH | |
| | | | VHQIAPGQTGVIADYNYKLPSEFVGCILAWNTRTIDSKRGFYYRLFRHGNIRPYERDTSNVPYNAAGG | |
| | | | TCNQPGTHNCYEPLQDYGFTSTSGVGYQPFRVVVLSFELLNAPATVCGPKQSTDLVKNKCVNFNFNG | |
| | | | LTGTGVLTDSNKKFQPFQQFGRDSADFTDSVKDPKTLEILDITPCSYGGVSVITPGTNTSDSVAVLYQD | |
| | | | VNCTDVPTMLHMDQVSNDWRVYAVNTDGNMFQTQAGCLVGATYDNTSYECDIPVGAGVCAKFQTT | |
| | | | GSASQSSILAYTMSLGEDSNVAYSNNSIAIPTNFTISVTTEVLPVSMTKTAVDCNMYICGDSTECSSLLL | |
| | | | QYGSFCQQLNRALAGVSVEQDKNTQDVFAQVKSIYKVSAIKDFGGFNFSQILPDPAKPSKRSFIEDLLY | |
| | | | NKVTLADPGFMKQYGDCLGGVNARDLICAQKFNGLTVLPPLLTDDMIAAYTSALVSGTALSGFTFGAG | |
| | | | AALQIPFAMQMAYRFNGIGVTQNVLYENQKQIANQFNKAISQIQESLTTTATALGKLQDVINQNAIALNT | |
| | | | LVKQLSSNFGAISSVLNDILSRLDPPEAEVQIDRLITGRLQSLQTYVTQQLIRAADIRASANLAATKMSEC | |
| | | | VLGQSKRVDFCGKGYHLMSFPQAAPHGVVFLHVTYVPSQDKNFTTAPAICHKGKAYFPREGVFVTNG | |
| | | | THWFVTQRNFYQPEVITTENTFESGNCDWVIGIVNNTVYDPLQSELDSIKEELDKIHKNGSGGSGDIIKL | |
| | | | LNEQVNKEMQSSNLYMSMSSWCYTHSLDGAGLFLFDHAAEEYEHAKKLIIFLNENNVPVQLTSISAPE | |
| | | | HKFEGLTQIFQKAYEHEQHISESINNIVDHAIKSKDHATFNFLQWYVAEQHEEEVLFKDILDKIELIGNEN | |
| | | | HGLYLADQYVKGIAKSRKSGS | |
| | SpFN | BANAL20-247 SpFN | MDSKGSSQKGSRLLLLLVVSNLLLPQGVVGQTTCVTGPTTENKQNVSSLMRGVYYPDDIYRSNVNVL | 482 |
| | | | FTVPFLRFNSTLTWYNFWNQAYTSRVMEFGDGIYFSTVDKSNAVRGWIFGTTLDNTTQSALLINNGSL | |
| | | | VTIQVCYFQFCANPAFLVAGGQQTSAAVYISSHNCTYSEVLSHISLDFTEDTGSFKRLREFVFKNNDGF | |
| | | | LHIYGAYEPHVIGIGVTPSLPSTFKPLIPLWKLPLGLNITNFKVVLTFRSNSQPLQANFAVGSLKLTTFML | |
| | | | SFDINGTIDKAVDCSSDPLSELKCTLKSFNVSKGIYPTSNFRVLPSTEVVRFPNITNFCPFDKVFNATRF | |
| | | | PNVYAWQRTKISDCIADYTVLYNSTSFSTFKCYGVSPSKLIDLCFTSVYADTFLIRFSEVRQIAPGETGVI | |
| | | | ADYNYKLPDDFTGCVLAWNTAQQDIGSYFYRSHRAVKLKPFERDLSSDENGVRTLSTYDFNPNVPLD | |
| | | | YQATRVVVLSFELLNAPATVCGPKLSTQLVKNRCVNFNFNGLRGTGVLTDSDKRFQSFQQFGRDSAD | |
| | | | FTDSVRDPQTLQILDISPCSFGGVSVITPGTQTSSKVAVLYQDVNCTDVPTALGLDQISAAWRVYAIGN | |
| | | | DVFQTQAGCLVGAEHTNISYECDIPIGAGVCASYNSGSASVGTNSIIAYAMSIGAESSIAYSNNSIAIPTN | |
| | | | FSIQVTTEVLPVSMSKTSVDCTMYICGDSQECNKLLLQYGSFCAQLNRALSGVAVEQDKNTEAVFAQV | |
| | | | KQIYKAPVIKDLGGFNFSQLLPDPTKPSQRSFIEDLLFDKVTLSDAGFIKQYGDCLGDIAARDLICAQKF | |
| | | | NGLTVLPPLLTDEMIAAYTSALVSGTATAGWTFGIGAALQVPFAMQMAYRFNGIGVTQNVLYENQKLIA | |
| | | | NQFNSAIGKIQDSLLSTASALGKLQDVVNQNAQALNTLVKQLSSNFGAISSVLNDILSRLDPPEAEVQID | |
| | | | RLITGRLQSLQTYVTQQLIRAAEIRASANLAATKMSECVLGQSKRVDFCGKGYHLMSFPQSAPHGVVF | |
| | | | LHVTYVPAQEKNFTTAPAICYDGKAHFPREGVFVSNGTHWFVTQRNFYEPQIITTDNTFVSGTCDVVI | |
| | | | GIVNNTVYDPLQSELDSIKEELDKIHKNGSGGSGDIIKLLNEQVNKEMQSSNLYMSMSSWCYTHSLDG | |
| | | | AGLFLFDHAAEEYEHAKKLIIFLNENNVPVQLTSISAPEHKFEGLTQIFQKAYEHEQHISESINNIVDHAIK | |
| | | | SKDHATFNFLQWYVAEQHEEEVLFKDILDKIELIGNENHGLYLADQYVKGIAKSRKSGS | |
| | RRFN | Beta-WA1 RRFN | MDSKGSSQKGSRLLLLLVVSNLLLPQGVVGQNITNLCPFGEVFNATRFASVYAWNRKRISNCVADYSV | 483 |
| | | | LYNSASFSTFKCYGVSPTKLNDLCFTNVYADSFVIRGDEVRQIAPGQTGNIADYNYKLPDDFTGCVIAW | |
| | | | NSNNLDSKVGGNYNYLRRLFRKSNLKPFERDISTEIYQAGSTPCNGVKGFNCYFPLQSYGFQPTYGV | |
| | | | GYQPYRVVVLSFENKSAPATVCGGGSGGSGNITNLCPFGEVFNATRFASVYAWNRKRISNCVADYSV | |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | RRFN | WA1-Beta RRFN | MDSKGSSQKGSRLLLLLVVSNLLLPQGVVGQNITNLCPFGEVFNATRFASVYAWNRKRISNCVADYSV | 484 |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNITNLCPFGEVFNATRFASVYAWNRKRISNCVADYSV | |
| | | | LYNSASFSTFKCYGVSPTKLNDLCFTNVYADSFVIRGDEVRQIAPGQTGNIADYNYKLPDDFTGCVIAW | |
| | | | NSNNLDSKVGGNYNYLRRLFRKSNLKPFERDISTEIYQAGSTPCNGVKGFNCYFPLQSYGFQPTYGV | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | RRFN | BA4/5-WA1 RRFN | MDSKGSSQKGSRLLLLLVVSNLLLPQGVVGQNITNLCPFDEVFNATRFASVYAWNRKRISNCVADYSV | 485 |
| | | | LYNFAPFFAFKCYGVSPTKLNDLCFTNVYADSFVIRGNEVSQIAPGQTGNIADYNYKLPDDFTGCVIAW | |
| | | | NSNKLDSKVGGNYNYRRRLFRKSNLKPFERDISTEIYQAGNKPCNGVAGVNCYFPLQSYGFRPTYGV | |
| | | | GHQPYRVVVLSFENKSAPATVCGGGSGGSGNITNLCPFGEVFNATRFASVYAWNRKRISNCVADYSV | |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | RRFN | WA1-BA4/5 RRFN | MDSKGSSQKGSRLLLLLVVSNLLLPQGWVGQNITNLCPFGEVFNATRFASVYAWNRKRISNCVADYSV | 486 |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNITNLCPFDEVFNATRFASVYAWNRKRISNCVADYSV | |
| | | | LYNFAPFFAFKCYGVSPTKLNDLCFTNVYADSFVIRGNEVSQIAPGQTGNIADYNYKLPDDFTGCVIAW | |
| | | | NSNKLDSKVGGNYNYRRRLFRKSNLKPFERDISTEIYQAGNKPCNGVAGVNCYFPLQSYGFRPTYGV | |
| | | | GHQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | RRFN | Khosta2-WA1 RRFN | MDSKGSSQKGSRLLLLLVVSNLLLPQGVVGQNMTNICPFDQVFNKTQFPSVYAWERVRISDCVSDYT | 487 |
| | | | VLYNSSASFSTFKCYGVSPTKLNDLCFSGVYADYFVVKGDHVHQIAPGQTGVIADYNYKLPSEFVGCIL | |
| | | | AWNTRTIDSKRGFYYRLFRHGNIRPYERDTSNVPYNAAGGTCNQPGTHNCYEPLQDYGFTSTSGVG | |
| | | | YQPFRVVVLSFENKSAPATVCGGGSGGSGNITNLCPFGEVFNATRFASVYAWNRKRISNCVADYSVL | |
| | | | YNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | RRFN | WA1-Khosta2 RRFN | MDSKGSSQKGSRLLLLLVVSNLLLPQGVVGQNITNLCPFGEVFNATRFASVYAWNRKRISNCVADYSV | 488 |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNMTNICPFDQVFNKTQFPSVYAWERVRISDCVSDYT | |
| | | | VLYNSSASFSTFKCYGVSPTKLNDLCFSGVYADYFVVKGDHVHQIAPGQTGVIADYNYKLPSEFVGCIL | |
| | | | AWNTRTIDSKRGFYYRLFRHGNIRPYERDTSNVPYNAAGGTCNQPGTHNCYEPLQDYGFTSTSGVG | |
| | | | YQPFRVVVLSFENKSAPATVCGGGSGGSGESQVRQQFSKDIEKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESI | |
| | | | NNIVDHAIKSKDHATFNFLQWYVAEQHEEEVLFKDILDKIELIGNENHGLYLADQYVKGIAKSRKSGS | |
| | RRFN | BANAL20247-WA1 RRFN | MDSKGSSQKGSRLLLLLVVSNLLLPQGVVGQNITNFCPFDKVFNATRFPNVYAWQRTKISDCIADYTVL | 489 |
| | | | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGNITNLCPFGEVFNATRFASVYAWNRKRISNCVADYSVLYNSASFSTFKCYGVSPTKLND | |
| | | | LCFTNVYADSFVIRGDEVRQIAPGQTGKIADYNYKLPDDFTGCVIAWNSNNLDSKVGGNYNYLRRLFR | |
| | | | KSNLKPFERDISTEIYQAGSTPCNGVEGFNCYFPLQSYGFQPTNGVGYQPYRVVVLSFENKSAPATVC | |
| | | | GGGSGGSGESQVRQQFSKDIEKLLNEQVNKEMQSSNLYMSMSSWCYTHSLDGAGLFLFDHAAEEY | |
| | | | EHAKKLIIFLNENNVPVQLTSISAPEHKFEGLTQIFQKAYEHEQHISESINNIVDHAIKSKDHATFNFLQW | |
| | | | YVAEQHEEEVLFKDILDKIELIGNENHGLYLADQYVKGIAKSRKSGS | |
| | RRFN | WA1-BANAL20247 RRFN | MDSKGSSQKGSRLLLLLVVSNLLLPQGVVGQNITNLCPFGEVFNATRFASVYAWNRKRISNCVADYSV | 490 |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNITNFCPFDKVFNATRFPNVYAWQRTKISDCIADYTVL | |
| | | | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGESQVRQQFSKDIEKLLNEQVNKEMQSSNLYMSMSSWCYTHSLDGAGLFLFDHAAEEY | |
| | | | EHAKKLIIFLNENNVPVQLTSISAPEHKFEGLTQIFQKAYEHEQHISESINNIVDHAIKSKDHATFNFLQW | |
| | | | YVAEQHEEEVLFKDILDKIELIGNENHGLYLADQYVKGIAKSRKSGS | |
| | R-SpFN | BA4/5-WA1 R-SpFN | MDSKGSSQKGSRLLLLLVVSNLLLPQGVVGQNITNLCPFDEVFNATRFASVYAWNRKRISNCVADYSV | 491 |
| | | | LYNFAPFFAFKCYGVSPTKLNDLCFTNVYADSFVIRGNEVSQIAPGQTGNIADYNYKLPDDFTGCVIAW | |
| | | | NSNKLDSKVGGNYNYRRRLFRKSNLKPFERDISTEIYQAGNKPCNGVAGVNCYFPLQSYGFRPTYGV | |
| | | | GHQPYRVVVLSFENKSAPATVCGGGSGGSGSSQCVNLTTRTQLPPAYTNSFTRGVYYPDKVFRSSV | |
| | | | LHSTQDLFLPFFSNVTWFHAIHVSGTNGTKRFDNPVLPFNDGVYFASTEKSNIIRGWIFGTTLDSKTQS | |
| | | | LLIVNNATNVVIKVCEFQFCNDPFLGVYYHKNNKSWMESEFRVYSSANNCTFEYVSQPFLMDLEGKQ | |
| | | | GNFKNLREFVFKNIDGYFKIYSKHTPINLVRDLPQGFSALEPLVDLPIGINITRFQTLLALHRSYLTPGDS | |
| | | | SSGWTAGAAAYYVGYLQPRTFLLKYNENGTITDAVDCALDPLSETKCTLKSFTVEKGIYQTSNFRVQP | |
| | | | TESIVRFPNITNLCPFGEVFNATRFASVYAWNRKRISNCVADYSVLYNSASFSTFKCYGVSPTKLNDLC | |
| | | | FTNVYADSFVIRGDEVRQIAPGQTGKIADYNYKLPDDFTGCVIAWNSNNLDSKVGGNYNYLYRLFRKS | |
| | | | NLKPFERDISTEIYQAGSTPCNGVEGFNCYFPLQSYGFQPTNGVGYQPYRVVVLSFELLHAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| | R-SpFN | BANAL 20247-WA1 R-SpFN | MDSKGSSQKGSRLLLLLVVSNLLLPQGVVGQNITNFCPFDKVFNATRFPNVYAWQRTKISDCIADYTVL | 492 |
| | | | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATRF | |
| | | | ASVYAWNRKRISNCVADYSVLYNSASFSTFKCYGVSPTKLNDLCFTNVYADSFVIRGDEVRQIAPGQT | |
| | | | GKIADYNYKLPDDFTGCVIAWNSNNLDSKVGGNYNYLYRLFRKSNLKPFERDISTEIYQAGSTPCNGV | |
| | | | EGFNCYFPLQSYGFQPTNGVGYQPYRVVVLSFELLHAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDWVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| | RR-mosSpFN | Khosta2-BANAL20247-SARS1- | MDSKGSSQKGSRLLLLLVVSNLLLPQGVVGQNMTNICPFDQVFNKTQFPSVYAWERVRISDCVSDYT | 493 |
| | | WA1 RR-mosSpFN | VLYNSSASFSTFKCYGVSPTKLNDLCFSGVYADYFVVKGDHVHQIAPGQTGVIADYNYKLPSEFVGCIL | |
| | | | AWNTRTIDSKRGFYYRLFRHGNIRPYERDTSNVPYNAAGGTCNQPGTHNCYEPLQDYGFTSTSGVG | |
| | | | YQPFRVVVLSFENKSAPATVCGGGSGGSGNITNFCPFDKVFNATRFPNVYAWQRTKISDCIADYTVLY | |
| | | | NSTSFSTFKCYGVSPSKLIDLCFTSVYADTFLIRFSEVRQIAPGETGVIADYNYKLPDDFTGCVLAWNTA | |
| | | | QQDIGSYFYRSHRAVKLKPFERDLSSDENGVRTLSTYDFNPNVPLDYQATRVVVLSFENKSAPATVCG | |
| | | | GGSGGSGSSQCVNLTTRTQLPPAYTNSFTRGVYYPDKVFRSSVLHSTQDLFLPFFSNVTWFHAIHVS | |
| | | | GTNGTKRFDNPVLPFNDGVYFASTEKSNIIRGWIFGTTLDSKTQSLLIVNNATNVVIKVCEFQFQNDPFL | |
| | | | GVYYHKNNKSWMESEFRVYSSANNCTFEYVSQPFLMDLEGKQGNFKNLREFVFKNIDGYFKIYSKHT | |
| | | | PINLVRDLPQGFSALEPLVDLPIGINITRFQTLLALHRSYLTPGDSSSGWTAGAAAYYVGYLQPRTFLLK | |
| | | | YNENGTITDAVDCALDPLSETKCTLKSFTVEKGIYQTSNFRVQPTESIVRFPNITNLCPFGEVFNATKFP | |
| | | | SVYAWERKKISNCVADYSVLYNSTFFSTFKCYGVSATKLNDLCFSNVYADSFVVKGDDVRQIAPGQTG | |
| | | | VIADYNYKLPDDFMGCVLAWNTRNIDATSTGNYNYKYRYLRHGKLRPFERDISNVPFSPDGKPCTPPA | |
| | | | LNCYWPLNDYGFYTTTGIGYQPYRVVVLSFELLNAPATVCGPKKSTNLVKNKCVNFNFNGLTGTGVLT | |
| | | | ESNKKFLPFQQFGRDIADTTDAVRDPQTLEILDITPCSFGGVSVITPGTNTSNQVAVLYQDVNCTEVPV | |
| | | | AIHADQLTPTWRVYSTGSNVFQTRAGCLIGAEHVNNSYECDIPIGAGICASYQTQTNSPGSASSVASQ | |
| | | | SIIAYTMSLGAENSVAYSNNSIAIPTNFTISVTTEILPVSMTKTSVDCTMYICGDSTECSNLLLQYGSFCT | |
| | | | QLNRALTGIAVEQDKNTQEVFAQVKQIYKTPPIKDFGGFNFSQILPDPSKPSKRSFIEDLLFNKVTLADA | |
| | | | GFIKQYGDCLGDIAARDLICAQKFNGLTVLPPLLTDEMIAQYTSALLAGTITSGWTFGAGAALQIPFAMQ | |
| | | | MAYRFNGIGVTQNVLYENQKLIANQFNSAIGKIQDSLSSTASALGKLQDVVNQNAQALNTLVKQLSSNF | |
| | | | GAISSVLNDILSRLDPPEAEVQIDRLITGRLQSLQTYVTQQLIRAAEIRASANLAATKMSECVLGQSKRV | |
| | | | DFCGKGYHLMSFPQSAPHGVVFLHVTYVPAQEKNFTTAPAICHDGKAHFPREGVFVSNGTHWFVTQ | |
| | | | RNFYEPQIITTDNTFVSGNCDWVIGIVNNTVYDPLQSELDSIKEELDKIHKNGSGGSGDIIKLLNEQVNKE | |
| | | | MQSSNLYMSMSSWCYTHSLDGAGLFLFDHAAEEYEHAKKLIIFLNENNVPVQLTSISAPEHKFEGLTQI | |
| | | | FQKAYEHEQHISESINNIVDHAIKSKDHATFNFLQWYVAEQHEEEVLFKDILDKIELIGNENHGLYLADQ | |
| | | | YVKGIAKSRKSGS | |
| | RR-mosSpFN | BANAL 20247-Khosta2-SARS1- | MDSKGSSQKGSRLLLLLVVSNLLLPQGVVGQNITNFCPFDKVFNATRFPNVYAWQRTKISDCIADYTVL | 494 |
| | | WA1 RR-mosSpFN | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGNMTNICPFDQVFNKTQFPSVYAWERVRISDCVSDYTVLYNSSASFSTFKCYGVSPTKLN | |
| | | | DLCFSGVYADYFWVKGDHVHQIAPGQTGVIADYNYKLPSEFVGCILAWNTRTIDSKRGFYYRLFRHGNI | |
| | | | RPYERDTSNVPYNAAGGTCNQPGTHNCYEPLQDYGFTSTSGVGYQPFRVVVLSFENKSAPATVCGG | |
| | | | GSGGSGSSQCVNLTTRTQLPPAYTNSFTRGVYYPDKVFRSSVLHSTQDLFLPFFSNVTWFHAIHVSG | |
| | | | TNGTKRFDNPVLPFNDGVYFASTEKSNIIRGWIFGTTLDSKTQSLLIVNNATNVVIKVCEFQFCNDPFLG | |
| | | | VYYHKNNKSWMESEFRVYSSANNCTFEYVSQPFLMDLEGKQGNFKNLREFVFKNIDGYFKIYSKHTPI | |
| | | | NLVRDLPQGFSALEPLVDLPIGINITRFQTLLALHRSYLTPGDSSSGWTAGAAAYYVGYLQPRTFLLKY | |
| | | | NENGTITDAVDCALDPLSETKCTLKSFTVEKGIYQTSNFRVQPTESIVRFPNITNLCPFGEVFNATKFPS | |
| | | | VYAWERKKISNCVADYSVLYNSTFFSTFKCYGVSATKLNDLCFSNVYADSFVVKGDDVRQIAPGQTGV | |
| | | | IADYNYKLPDDFMGCVLAWNTRNIDATSTGNYNYKYRYLRHGKLRPFERDISNVPFSPDGKPCTPPAL | |
| | | | NCYWPLNDYGFYTTTGIGYQPYRVVVLSFELLNAPATVCGPKKSTNLVKNKCVNFNFNGLTGTGVLTE | |
| | | | SNKKFLPFQQFGRDIADTTDAVRDPQTLEILDITPCSFGGVSVITPGTNTSNQVAVLYQDVNCTEVPVAI | |
| | | | HADQLTPTWRVYSTGSNVFQTRAGCLIGAEHVNNSYECDIPIGAGICASYQTQTNSPGSASSVASQSII | |
| | | | AYTMSLGAENSVAYSNNSIAIPTNFTISVTTEILPVSMTKTSVDCTMYICGDSTECSNLLLQYGSFCTQL | |
| | | | NRALTGIAVEQDKNTQEVFAQVKQIYKTPPIKDFGGFNFSQILPDPSKPSKRSFIEDLLFNKVTLADAGFI | |
| | | | KQYGDCLGDIAARDLICAQKFNGLTVLPPLLTDEMIAQYTSALLAGTITSGWTFGAGAALQIPFAMQMA | |
| | | | YRFNGIGVTQNVLYENQKLIANQFNSAIGKIQDSLSSTASALGKLQDVVNQNAQALNTLVKQLSSNFGA | |
| | | | ISSVLNDILSRLDPPEAEVQIDRLITGRLQSLQTYVTQQLIRAAEIRASANLAATKMSECVLGQSKRVDFC | |
| | | | GKGYHLMSFPQSAPHGVVFLHVTYVPAQEKNFTTAPAICHDGKAHFPREGVFVSNGTHWFVTQRNF | |
| | | | YEPQIITTDNTFVSGNCDWVIGIVNNTVYDPLQSELDSIKEELDKIHKNGSGGSGDIIKLLNEQVNKEMQS | |
| | | | SNLYMSMSSWCYTHSLDGAGLFLFDHAAEEYEHAKKLIIFLNENNVPVQLTSISAPEHKFEGLTQIFQK | |
| | | | AYEHEQHISESINNIVDHAIKSKDHATFNFLQWYVAEQHEEEVLFKDILDKIELIGNENHGLYLADQYVK | |
| | | | GIAKSRKSGS | |
| | RR-mosSpFN | BANAL 20247-SARS1-Khosta2- | MDSKGSSQKGSRLLLLLVVSNLLLPQGWVGQNITNFCPFDKVFNATRFPNVYAWQRTKISDCIADYTVL | 495 |
| | | WA1 RR-mosSpFN | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGNITNLCPFGEVFNATKFPSVYAWERKKISNCVADYSVLYNSTFFSTFKCYGVSATKLNDL | |
| | | | CFSNVYADSFVVKGDDVRQIAPGQTGVIADYNYKLPDDFMGCVLAWNTRNIDATSTGNYNYKRRYLR | |
| | | | HGKLRPFERDISNVPFSPDGKPCTPPALNCYWPLNDYGFYTTTGIGYQPYRVVVLSFENKSAPATVCG | |
| | | | GGSGGSGSSQCVNLTTRTQLPPAYTNSFTRGVYYPDKVFRSSVLHSTQDLFLPFFSNVTWFHAIHVS | |
| | | | GTNGTKRFDNPVLPFNDGVYFASTEKSNIIRGWIFGTTLDSKTQSLLIVNNATNVVIKVCEFQFCNDPFL | |
| | | | GVYYHKNNKSWMESEFRVYSSANNCTFEYVSQPFLMDLEGKQGNFKNLREFVFKNIDGYFKIYSKHT | |
| | | | PINLVRDLPQGFSALEPLVDLPIGINITRFQTLLALHRSYLTPGDSSSGWTAGAAAYYVGYLQPRTFLLK | |
| | | | YNENGTITDAVDCALDPLSETKCTLKSFTVEKGIYQTSNFRVQPTESIVRFPNMTNICPFDQVFNKTQF | |
| | | | PSVYAWERVRISDCVSDYTVLYNSSASFSTFKCYGVSPTKLNDLCFSGVYADYFVVKGDHVHQIAPG | |
| | | | QTGVIADYNYKLPSEFVGCILAWNTRTIDSKRGFYYRLFRHGNIRPYERDTSNVPYNAAGGTCNQPGT | |
| | | | HNCYEPLQDYGFTSTSGVGYQPFRVVVLSFELLNAPATVCGPKKSTNLVKNKCVNFNFNGLTGTGVL | |
| | | | TESNKKFLPFQQFGRDIADTTDAVRDPQTLEILDITPCSFGGVSVITPGTNTSNQVAVLYQDVNCTEVP | |
| | | | VAIHADQLTPTWRVYSTGSNVFQTRAGCLIGAEHVNNSYECDIPIGAGICASYQTQTNSPGSASSVAS | |
| | | | QSIIAYTMSLGAENSVAYSNNSIAIPTNFTISVTTEILPVSMTKTSVDCTMYICGDSTECSNLLLQYGSFC | |
| | | | TQLNRALTGIAVEQDKNTQEVFAQVKQIYKTPPIKDFGGFNFSQILPDPSKPSKRSFIEDLLFNKVTLAD | |
| | | | AGFIKQYGDCLGDIAARDLICAQKFNGLTVLPPLLTDEMIAQYTSALLAGTITSGWTFGAGAALQIPFAM | |
| | | | QMAYRFNGIGVTQNVLYENQKLIANQFNSAIGKIQDSLSSTASALGKLQDVVNQNAQALNTLVKQLSS | |
| | | | NFGAISSVLNDILSRLDPPEAEVQIDRLITGRLQSLQTYVTQQLIRAAEIRASANLAATKMSECVLGQSK | |
| | | | RVDFCGKGYHLMSFPQSAPHGVVFLHVTYVPAQEKNFTTAPAICHDGKAHFPREGVFVSNGTHWFV | |
| | | | TQRNFYEPQIITTDNTFVSGNCDVVIGIVNNTVYDPLQSELDSIKEELDKIHKNGSGGSGDIIKLLNEQVN | |
| | | | KEMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| | RR-mosSpFN | SARS1-BANAL20247-Khosta2- | MDSKGSSQKGSRLLLLLVVSNLLLPQGVVGQNITNLCPFGEVFNATKFPSVYAWERKKISNCVADYSV | 496 |
| | | WA1 RR-mosSpFN | LYNSTFFSTFKCYGVSATKLNDLCFSNVYADSFVVKGDDVRQIAPGQTGVIADYNYKLPDDFMGCVLA | |
| | | | WNTRNIDATSTGNYNYKRRYLRHGKLRPFERDISNVPFSPDGKPCTPPALNCYWPLNDYGFYTTTGI | |
| | | | GYQPYRVVVLSFENKSAPATVCGGGSGGSGNITNFCPFDKVFNATRFPNVYAWQRTKISDCIADYTVL | |
| | | | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNMTNICPFDQVFNKTQ | |
| | | | FPSVYAWERVRISDCVSDYTVLYNSSASFSTFKCYGVSPTKLNDLCFSGVYADYFVVKGDHVHQIAPG | |
| | | | QTGVIADYNYKLPSEFVGCILAWNTRTIDSKRGFYYRLFRHGNIRPYERDTSNVPYNAAGGTCNQPGT | |
| | | | HNCYEPLQDYGFTSTSGVGYQPFRVVVLSFELLNAPATVCGPKKSTNLVKNKCVNFNFNGLTGTGVL | |
| | | | TESNKKFLPFQQFGRDIADTTDAVRDPQTLEILDITPCSFGGVSVITPGTNTSNQVAVLYQDVNCTEVP | |
| | | | VAIHADQLTPTWRVYSTGSNVFQTRAGCLIGAEHVNNSYECDIPIGAGICASYQTQTNSPGSASSVAS | |
| | | | QSIIAYTMSLGAENSVAYSNNSIAIPTNFTISVTTEILPVSMTKTSVDCTMYICGDSTECSNLLLQYGSFC | |
| | | | TQLNRALTGIAVEQDKNTQEVFAQVKQIYKTPPIKDFGGFNFSQILPDPSKPSKRSFIEDLLFNKVTLAD | |
| | | | AGFIKQYGDCLGDIAARDLICAQKFNGLTVLPPLLTDEMIAQYTSALLAGTITSGWTFGAGAALQIPFAM | |
| | | | QMAYRFNGIGVTQNVLYENQKLIANQFNSAIGKIQDSLSSTASALGKLQDVVNQNAQALNTLVKQLSS | |
| | | | NFGAISSVLNDILSRLDPPEAEVQIDRLITGRLQSLQTYVTQQLIRAAEIRASANLAATKMSECVLGQSK | |
| | | | RVDFCGKGYHLMSFPQSAPHGVVFLHVTYVPAQEKNFTTAPAICHDGKAHFPREGVFVSNGTHWFV | |
| | | | TQRNFYEPQIITTDNTFVSGNCDVVIGIVNNTVYDPLQSELDSIKEELDKIHKNGSGGSGDIIKLLNEQVN | |
| | | | KEMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| | RR-SpFN | BANAL20247-SARS1-WA1 RR- | MDSKGSSQKGSRLLLLLVVSNLLLPQGVVGQNITNFCPFDKVFNATRFPNVYAWQRTKISDCIADYTVL | 497 |
| | | SpFN | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFELLNAPATVC | |
| | | | GGGSGGSGNITNLCPFGEVFNATKFPSVYAWERKKISNCVADYSVLYNSTFFSTFKCYGVSATKLNDL | |
| | | | CFSNVYADSFWVKGDDVRQIAPGQTGVIADYNYKLPDDFMGCVLAWNTRNIDATSTGNYNYKRRYLR | |
| | | | HGKLRPFERDISNVPFSPDGKPCTPPALNCYWPLNDYGFYTTTGIGYQPYRVVVLSFENKSAPATVCG | |
| | | | GGSGGSGSSQCVNLTTRTQLPPAYTNSFTRGVYYPDKVFRSSVLHSTQDLFLPFFSNVTWFHAIHVS | |
| | | | GTNGTKRFDNPVLPFNDGVYFASTEKSNIIRGWIFGTTLDSKTQSLLIVNNATNVVIKVCEFQFCNDPFL | |
| | | | GVYYHKNNKSWMESEFRVYSSANNCTFEYVSQPFLMDLEGKQGNFKNLREFVFKNIDGYFKIYSKHT | |
| | | | PINLVRDLPQGFSALEPLVDLPIGINITRFQTLLALHRSYLTPGDSSSGWTAGAAAYYVGYLQPRTFLLK | |
| | | | YNENGTITDAVDCALDPLSETKCTLKSFTVEKGIYQTSNFRVQPTESIVRFPNITNLCPFGEVFNATRFA | |
| | | | SVYAWNRKRISNCVADYSVLYNSASFSTFKCYGVSPTKLNDLCFTNVYADSFVIRGDEVRQIAPGQTG | |
| | | | KIADYNYKLPDDFTGCVIAWNSNNLDSKVGGNYNYLYRLFRKSNLKPFERDISTEIYQAGSTPCNGVE | |
| | | | GFNCYFPLQSYGFQPTNGVGYQPYRVVVLSFELLHAPATVCGPKKSTNLVKNKCVNFNFNGLTGTGV | |
| | | | LTESNKKFLPFQQFGRDIADTTDAVRDPQTLEILDITPCSFGGVSVITPGTNTSNQVAVLYQDVNCTEV | |
| | | | PVAIHADQLTPTWRVYSTGSNVFQTRAGCLIGAEHVNNSYECDIPIGAGICASYQTQTNSPGSASSVA | |
| | | | SQSIIAYTMSLGAENSVAYSNNSIAIPTNFTISVTTEILPVSMTKTSVDCTMYICGDSTECSNLLLQYGSF | |
| | | | CTQLNRALTGIAVEQDKNTQEVFAQVKQIYKTPPIKDFGGFNFSQILPDPSKPSKRSFIEDLLFNKVTLA | |
| | | | DAGFIKQYGDCLGDIAARDLICAQKFNGLTVLPPLLTDEMIAQYTSALLAGTITSGWTFGAGAALQIPFA | |
| | | | MQMAYRFNGIGVTQNVLYENQKLIANQFNSAIGKIQDSLSSTASALGKLQDVVNQNAQALNTLVKQLS | |
| | | | SNFGAISSVLNDILSRLDPPEAEVQIDRLITGRLQSLQTYVTQQLIRAAEIRASANLAATKMSECVLGQSK | |
| | | | RVDFCGKGYHLMSFPQSAPHGVVFLHVTYVPAQEKNFTTAPAICHDGKAHFPREGVFVSNGTHWFV | |
| | | | TQRNFYEPQIITTDNTFVSGNCDVVIGIVNNTVYDPLQSELDSIKEELDKIHKNGSGGSGDIIKLLNEQVN | |
| | | | KEMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| | RR-SpFN | SARS1-BANAL20247-WA1 RR- | MDSKGSSQKGSRLLLLLVVSNLLLPQGWVGQNITNLCPFGEVFNATKFPSVYAWERKKISNCVADYSV | 498 |
| | | SpFN | LYNSTFFSTFKCYGVSATKLNDLCFSNVYADSFVVKGDDVRQIAPGQTGVIADYNYKLPDDFMGCVLA | |
| | | | WNTRNIDATSTGNYNYKRRYLRHGKLRPFERDISNVPFSPDGKPCTPPALNCYWPLNDYGFYTTTGI | |
| | | | GYQPYRVVVLSFENKSAPATVCGGGSGGSGNITNFCPFDKVFNATRFPNVYAWQRTKISDCIADYTVL | |
| | | | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFELLNAPATVC | |
| | | | GGGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATRF | |
| | | | ASVYAWNRKRISNCVADYSVLYNSASFSTFKCYGVSPTKLNDLCFTNVYADSFVIRGDEVRQIAPGQT | |
| | | | GKIADYNYKLPDDFTGCVIAWNSNNLDSKVGGNYNYLYRLFRKSNLKPFERDISTEIYQAGSTPCNGV | |
| | | | EGFNCYFPLQSYGFQPTNGVGYQPYRVVVLSFELLHAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDWVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| | S-2P | KHOSTA2mRNA_S2P | MDSKGSSQKGSRLLLLLVVSNLLLPQGVVGQQDGCATLSTKLTPTQLQVNSTRRGFYYFDDIYRSSLR | 499 |
| | | | VLVPGYFLPFGTNITRYQAQVINWTAIFFDNPVIPFNDGVYFAALEHSNWVRGWIFGTTLDNTTQSAIVV | |
| | | | NNATHILISVCYFNVCKEPMFAVSNFKPYKSWVYDRANNCTFNRAYPLNISTTPEPGKFKALREQVFR | |
| | | | YQDGFLYIYHSYESINSDTLPPGFSVLKPMLKLPLGLNITRFRVVMAMHSLTTSNFNTHSVNYFVGHLK | |
| | | | PLTMLVEFSPNGTIIDAIDCSQDPLSELKCTTKSFSVEKGIYQTSNFRVSPSLDWVRFPNMTNICPFDQV | |
| | | | FNKTQFPSVYAWERVRISDCVSDYTVLYNSSASFSTFKCYGVSPTKLNDLCFSGVYADYFVVKGDHV | |
| | | | HQIAPGQTGVIADYNYKLPSEFVGCILAWNTRTIDSKRGFYYRLFRHGNIRPYERDTSNVPYNAAGGT | |
| | | | CNQPGTHNCYEPLQDYGFTSTSGVGYQPFRVVVLSFELLNAPATVCGPKQSTDLVKNKCVNFNFNGL | |
| | | | TGTGVLTDSNKKFQPFQQFGRDSADFTDSVKDPKTLEILDITPCSYGGVSVITPGTNTSDSVAVLYQDV | |
| | | | NCTDVPTMLHMDQVSNDWRVYAVNTDGNMFQTQAGCLVGATYDNTSYECDIPVGAGVCAKFQTTT | |
| | | | RAKQSSILAYTMSLGEDSNVAYSNNSIAIPTNFTISVTTEVLPVSMTKTAVDCNMYICGDSTECSSLLLQ | |
| | | | YGSFCQQLNRALAGVSVEQDKNTQDVFAQVKSIYKVSAIKDFGGFNFSQILPDPAKPSKRSFIEDLLYN | |
| | | | KVTLADPGFMKQYGDCLGGVNARDLICAQKFNGLTVLPPLLTDDMIAAYTSALVSGTALSGFTFGAGA | |
| | | | ALQIPFAMQMAYRFNGIGVTQNVLYENQKQIANQFNKAISQIQESLTTTATALGKLQDVINQNAIALNTL | |
| | | | VKQLSSNFGAISSVLNDILSRLDPPEAEVQIDRLITGRLQSLQTYVTQQLIRAADIRASANLAATKMSECV | |
| | | | LGQSKRVDFCGKGYHLMSFPQAAPHGVVFLHVTYVPSQDKNFTTAPAICHKGKAYFPREGVFVTNGT | |
| | | | HWFVTQRNFYQPEVITTENTFESGNCDVVIGIVNNTVYDPLQPELESFKEELDKYFKNHTSPDVDFDDI | |
| | | | SGINASVVDIKKEIEHLNEIAKSLNESLIDLQELGKYEQYIKWPWYVWLGFIAGLIAIIMATIMLCCMTSCC | |
| | | | SCLKGLCSCGSCCKFDDDHSEPVLKGVKLHY | |
| | S-2P | BANAL20247mRNA_S2P | MDSKGSSQKGSRLLLLLVVSNLLLPQGVVGQTTCVTGPTTENKQNVSSLMRGVYYPDDIYRSNVNVL | 500 |
| | | | FTVPFLRFNSTLTWYNFWNQAYTSRVMEFGDGIYFSTVDKSNAVRGWIFGTTLDNTTQSALLINNGSL | |
| | | | VTIQVCYFQFCANPAFLVAGGQQTSAAVYISSHNCTYSEVLSHISLDFTEDTGSFKRLREFVFKNNDGF | |
| | | | LHIYGAYEPHVIGIGVTPSLPSTFKPLIPLWKLPLGLNITNFKVVLTFRSNSQPLQANFAVGSLKLTTFML | |
| | | | SFDINGTIDKAVDCSSDPLSELKCTLKSFNVSKGIYPTSNFRVLPSTEVVRFPNITNFCPFDKVFNATRF | |
| | | | PNVYAWQRTKISDCIADYTVLYNSTSFSTFKCYGVSPSKLIDLCFTSVYADTFLIRFSEVRQIAPGETGVI | |
| | | | ADYNYKLPDDFTGCVLAWNTAQQDIGSYFYRSHRAVKLKPFERDLSSDENGVRTLSTYDFNPNVPLD | |
| | | | YQATRVVVLSFELLNAPATVCGPKLSTQLVKNRCVNFNFNGLRGTGVLTDSDKRFQSFQQFGRDSAD | |
| | | | FTDSVRDPQTLQILDISPCSFGGVSVITPGTQTSSKVAVLYQDVNCTDVPTALGLDQISAAWRVYAIGN | |
| | | | DVFQTQAGCLVGAEHTNISYECDIPIGAGVCASYNSPAARVGTNSIIAYAMSIGAESSIAYSNNSIAIPTN | |
| | | | FSIQVTTEVLPVSMSKTSVDCTMYICGDSQECNKLLLQYGSFCAQLNRALSGVAVEQDKNTEAVFAQV | |
| | | | KQIYKAPVIKDLGGFNFSQLLPDPTKPSQRSFIEDLLFDKVTLSDAGFIKQYGDCLGDIAARDLICAQKF | |
| | | | NGLTVLPPLLTDEMIAAYTSALVSGTATAGWTFGIGAALQVPFAMQMAYRFNGIGVTQNVLYENQKLIA | |
| | | | NQFNSAIGKIQDSLLSTASALGKLQDVVNQNAQALNTLVKQLSSNFGAISSVLNDILSRLDPPEAEVQID | |
| | | | RLITGRLQSLQTYVTQQLIRAAEIRASANLAATKMSECVLGQSKRVDFCGKGYHLMSFPQSAPHGVVF | |
| | | | LHVTYVPAQEKNFTTAPAICYDGKAHFPREGVFVSNGTHWFVTQRNFYEPQIITTDNTFVSGTCDWVI | |
| | | | GIVNNTVYDPLQPELDSFKEELDKYFKNHTSPDVDLGDISGINASVVNIQKEIDRLNEVAKNLNESLIDLQ | |
| | | | ELGKYEQYIKWPWYIWLGFIAGLIAIVMVTIMLCCMTSCCSCLKGCCSCGSCCKFDEDDSEPVLNGVK | |
| | | | LHY | |
| | RR-mosSpFN | WA1-BANAL20247-SARS1-WA1 | MDSKGSSQKGSRLLLLLVVSNLLLPQGVVGQNITNLCPFGEVFNATRFASVYAWNRKRISNCVADYSV | 501 |
| | | RR-mosSpFN | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNITNFCPFDKVFNATRFPNVYAWQRTKISDCIADYTVL | |
| | | | YNSTSFSTFKCYGVSPSKLIDLCFTSVYADTFLIRFSEVRQIAPGETGVIADYNYKLPDDFTGCVLAWNT | |
| | | | AQQDIGSYFYRSHRAVKLKPFERDLSSDENGVRTLSTYDFNPNVPLDYQATRVVVLSFENKSAPATVC | |
| | | | GGGSGGSGSSQCVNLTTRTQLPPAYTNSFTRGVYYPDKVFRSSVLHSTQDLFLPFFSNVTWFHAIHV | |
| | | | SGTNGTKRFDNPVLPFNDGVYFASTEKSNIIRGWIFGTTLDSKTQSLLIVNNATNVVIKVCEFQFCNDPF | |
| | | | LGVYYHKNNKSWMESEFRVYSSANNCTFEYVSQPFLMDLEGKQGNFKNLREFVFKNIDGYFKIYSKH | |
| | | | TPINLVRDLPQGFSALEPLVDLPIGINITRFQTLLALHRSYLTPGDSSSGWTAGAAAYYVGYLQPRTFLL | |
| | | | KYNENGTITDAVDCALDPLSETKCTLKSFTVEKGIYQTSNFRVQPTESIVRFPNITNLCPFGEVFNATKF | |
| | | | PSVYAWERKKISNCVADYSVLYNSTFFSTFKCYGVSATKLNDLCFSNVYADSFVVKGDDVRQIAPGQT | |
| | | | GVIADYNYKLPDDFMGCVLAWNTRNIDATSTGNYNYKYRYLRHGKLRPFERDISNVPFSPDGKPCTPP | |
| | | | ALNCYWPLNDYGFYTTTGIGYQPYRVVVLSFELLNAPATVCGPKKSTNLVKNKCVNFNFNGLTGTGVL | |
| | | | TESNKKFLPFQQFGRDIADTTDAVRDPQTLEILDITPCSFGGVSVITPGTNTSNQVAVLYQDVNCTEVP | |
| | | | VAIHADQLTPTWRVYSTGSNVFQTRAGCLIGAEHVNNSYECDIPIGAGICASYQTQTNSPGSASSVAS | |
| | | | QSIIAYTMSLGAENSVAYSNNSIAIPTNFTISVTTEILPVSMTKTSVDCTMYICGDSTECSNLLLQYGSFC | |
| | | | TQLNRALTGIAVEQDKNTQEVFAQVKQIYKTPPIKDFGGFNFSQILPDPSKPSKRSFIEDLLFNKVTLAD | |
| | | | AGFIKQYGDCLGDIAARDLICAQKFNGLTVLPPLLTDEMIAQYTSALLAGTITSGWTFGAGAALQIPFAM | |
| | | | QMAYRFNGIGVTQNVLYENQKLIANQFNSAIGKIQDSLSSTASALGKLQDVVNQNAQALNTLVKQLSS | |
| | | | NFGAISSVLNDILSRLDPPEAEVQIDRLITGRLQSLQTYVTQQLIRAAEIRASANLAATKMSECVLGQSK | |
| | | | RVDFCGKGYHLMSFPQSAPHGVVFLHVTYVPAQEKNFTTAPAICHDGKAHFPREGVFVSNGTHWFV | |
| | | | TQRNFYEPQIITTDNTFVSGNCDVVIGIVNNTVYDPLQSELDSIKEELDKIHKNGSGGSGDIIKLLNEQVN | |
| | | | KEMQSSNLYMSMSSWCYTHSLDGAGLFLFDHAAEEYEHAKKLIIFLNENNVPVQLTSISAPEHKFEGLT | |
| | | | QIFQKAYEHEQHISESINNIVDHAIKSKDHATFNFLQWYVAEQHEEEVLFKDILDKIELIGNENHGLYLAD | |
| | | | QYVKGIAKSRKSGS | |
| | RR-mosSpFN | WA1-SARS1-BANAL20247-WA1 | MDSKGSSQKGSRLLLLLVVSNLLLPQGVVGQNITNLCPFGEVFNATRFASVYAWNRKRISNCVADYSV | 502 |
| | | RR-mosSpFN | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNITNLCPFGEVFNATKFPSVYAWERKKISNCVADYSV | |
| | | | LYNSTFFSTFKCYGVSATKLNDLCFSNVYADSFVVKGDDVRQIAPGQTGVIADYNYKLPDDFMGCVLA | |
| | | | WNTRNIDATSTGNYNYKRRYLRHGKLRPFERDISNVPFSPDGKPCTPPALNCYWPLNDYGFYTTTGI | |
| | | | GYQPYRVVVLSFENKSAPATVCGGGSGGSGSSQCVNLTTRTQLPPAYTNSFTRGVYYPDKVFRSSVL | |
| | | | HSTQDLFLPFFSNVTWFHAIHVSGTNGTKRFDNPVLPFNDGVYFASTEKSNIIRGWIFGTTLDSKTQSL | |
| | | | LIVNNATNVVIKVCEFQFCNDPFLGVYYHKNNKSWMESEFRVYSSANNCTFEYVSQPFLMDLEGKQG | |
| | | | NFKNLREFVFKNIDGYFKIYSKHTPINLVRDLPQGFSALEPLVDLPIGINITRFQTLLALHRSYLTPGDSSS | |
| | | | GWTAGAAAYYVGYLQPRTFLLKYNENGTITDAVDCALDPLSETKCTLKSFTVEKGIYQTSNFRVQPTE | |
| | | | SIVRFPNITNFCPFDKVFNATRFPNVYAWQRTKISDCIADYTVLYNSTSFSTFKCYGVSPSKLIDLCFTS | |
| | | | VYADTFLIRFSEVRQIAPGETGVIADYNYKLPDDFTGCVLAWNTAQQDIGSYFYRSHRAVKLKPFERDL | |
| | | | SSDENGVRTLSTYDFNPNVPLDYQATRVVVLSFELLNAPATVCGPKKSTNLVKNKCVNFNFNGLTGTG | |
| | | | VLTESNKKFLPFQQFGRDIADTTDAVRDPQTLEILDITPCSFGGVSVITPGTNTSNQVAVLYQDVNCTE | |
| | | | VPVAIHADQLTPTWRVYSTGSNVFQTRAGCLIGAEHVNNSYECDIPIGAGICASYQTQTNSPGSASSV | |
| | | | ASQSIIAYTMSLGAENSVAYSNNSIAIPTNFTISVTTEILPVSMTKTSVDCTMYICGDSTECSNLLLQYGS | |
| | | | FCTQLNRALTGIAVEQDKNTQEVFAQVKQIYKTPPIKDFGGFNFSQILPDPSKPSKRSFIEDLLFNKVTL | |
| | | | ADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQL | |
| | | | SSNFGAISSVLNDILSRLDPPEAEVQIDRLITGRLQSLQTYVTQQLIRAAEIRASANLAATKMSECVLGQS | |
| | | | KRVDFCGKGYHLMSFPQSAPHGVVFLHVTYVPAQEKNFTTAPAICHDGKAHFPREGVFVSNGTHWF | |
| | | | VTQRNFYEPQIITTDNTFVSGNCDVVIGIVNNTVYDPLQSELDSIKEELDKIHKNGSGGSGDIIKLLNEQV | |
| | | | NKEMQSSNLYMSMSSWCYTHSLDGAGLFLFDHAAEEYEHAKKLIIFLNENNVPVQLTSISAPEHKFEG | |
| | | | LTQIFQKAYEHEQHISESINNIVDHAIKSKDHATFNFLQWYVAEQHEEEVLFKDILDKIELIGNENHGLYL | |
| | | | ADQYVKGIAKSRKSGS | |
| | | SARS1-WA1 RRFN | MDSKGSSQKGSRLLLLLVVSNLLLPQGVVGQNITNLCPFGEVFNATKFPSVYAWERKKISNCVADYSV | 503 |
| | | | LYNSTFFSTFKCYGVSATKLNDLCFSNVYADSFVVKGDDVRQIAPGQTGVIADYNYKLPDDFMGCVLA | |
| | | | WNTRNIDATSTGNYNYKRRYLRHGKLRPFERDISNVPFSPDGKPCTPPALNCYWPLNDYGFYTTTGI | |
| | | | GYQPYRVVVLSFENKSAPATVCGGGSGGSGNITNLCPFGEVFNATRFASVYAWNRKRISNCVADYSV | |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | | WA1-SARS1 RRFN | MDSKGSSQKGSRLLLLLVVSNLLLPQGVVGQNITNLCPFGEVFNATRFASVYAWNRKRISNCVADYSV | 504 |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNITNLCPFGEVFNATKFPSVYAWERKKISNCVADYSV | |
| | | | LYNSTFFSTFKCYGVSATKLNDLCFSNVYADSFVVKGDDVRQIAPGQTGVIADYNYKLPDDFMGCVLA | |
| | | | WNTRNIDATSTGNYNYKRRYLRHGKLRPFERDISNVPFSPDGKPCTPPALNCYWPLNDYGFYTTTGI | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | | BQ1.1 RFN | MDSKGSSQKGSRLLLLLVVSNLLLPQGVVGQNITNLCPFDEVFNATTFASVYAWNRKRISNCVADYSV | 505 |
| | | | LYNFAPFFAFKCYGVSPTKLNDLCFTNVYADSFVIRGNEVSQIAPGQTGNIADYNYKLPDDFTGCVIAW | |
| | | | NSNKLDSTVGGNYNYRRRLFRKSKLKPFERDISTEIYQAGNKPCNGVAGVNCYFPLQSYGFRPTYGV | |
| | | | GHQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | | BQ1.1 SpFN | MDSKGSSQKGSRLLLLLVVSNLLLPQGVVGQSSQCVNLITRTQSYTNSFTRGVYYPDKVFRSSVLHST | 506 |
| | | | QDLFLPFFSNVTWFHAISGTNGTKRFDNPVLPFNDGVYFASTEKSNIIRGWIFGTTLDSKTQSLLIVNNA | |
| | | | TNVVIKVCEFQFCNDPFLDVYYHKNNKSWMESEFRVYSSANNCTFEYVSQPFLMDLEGKQGNFKNLR | |
| | | | EFVFKNIDGYFKIYSKHTPINLGRDLPQGFSALEPLVDLPIGINITRFQTLLALHRSYLTPGDSSSGWTAG | |
| | | | AAAYYVGYLQPRTFLLKYNENGTITDAVDCALDPLSETKCTLKSFTVEKGIYQTSNFRVQPTESIVRFPN | |
| | | | ITNLCPFDEVFNATTFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSTVGGNYNYRYRLFRKSKLKPFERDIS | |
| | | | TEIYQAGNKPCNGVAGVNCYFPLQSYGFRPTYGVGHQPYRVVVLSFELLHAPATVCGPKKSTNLVKN | |
| | | | KCVNFNFNGLTGTGVLTESNKKFLPFQQFGRDIADTTDAVRDPQTLEILDITPCSFGGVSVITPGTNTS | |
| | | | NQVAVLYQGVNCTEVPVAIHADQLTPTWRVYSTGSNVFQTRAGCLIGAEYVNNSYECDIPIGAGICAS | |
| | | | YQTQTKSHGSASSVASQSIIAYTMSLGAENSVAYSNNSIAIPTNFTISVTTEILPVSMTKTSVDCTMYICG | |
| | | | DSTECSNLLLQYGSFCTQLKRALTGIAVEQDKNTQEVFAQVKQIYKTPPIKYFGGFNFSQILPDPSKPS | |
| | | | KRSFIEDLLFNKVTLADAGFIKQYGDCLGDIAARDLICAQKFNGLTVLPPLLTDEMIAQYTSALLAGTITS | |
| | | | GWTFGAGAALQIPFAMQMAYRFNGIGVTQNVLYENQKLIANQFNSAIGKIQDSLSSTASALGKLQDWV | |
| | | | NHNAQALNTLVKQLSSKFGAISSVLNDILSRLDPPEAEVQIDRLITGRLQSLQTYVTQQLIRAAEIRASAN | |
| | | | LAATKMSECVLGQSKRVDFCGKGYHLMSFPQSAPHGVVFLHVTYVPAQEKNFTTAPAICHDGKAHFP | |
| | | | REGVFVSNGTHWFVTQRNFYEPQIITTDNTFVSGNCDVVIGIVNNTVYDPLQSELDSIKEELDKIHKNG | |
| | | | SGGSGDIIKLLNEQVNKEMQSSNLYMSMSSWCYTHSLDGAGLFLFDHAAEEYEHAKKLIIFLNENNVP | |
| | | | VQLTSISAPEHKFEGLTQIFQKAYEHEQHISESINNIVDHAIKSKDHATFNFLQWYVAEQHEEEVLFKDIL | |
| | | | DKIELIGNENHGLYLADQYVKGIAKSRKSGS | |
| | | BQ1.1-WA1 RRFN | MDSKGSSQKGSRLLLLLVVSNLLLPQGVVGQNITNLCPFDEVFNATTFASVYAWNRKRISNCVADYSV | 507 |
| | | | LYNFAPFFAFKCYGVSPTKLNDLCFTNVYADSFVIRGNEVSQIAPGQTGNIADYNYKLPDDFTGCVIAW | |
| | | | NSNKLDSTVGGNYNYRRRLFRKSKLKPFERDISTEIYQAGNKPCNGVAGVNCYFPLQSYGFRPTYGV | |
| | | | GHQPYRVVVLSFENKSAPATVCGGGSGGSGNITNLCPFGEVFNATRFASVYAWNRKRISNCVADYSV | |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | | WA1-BQ1.1 RRFN | MDSKGSSQKGSRLLLLLVVSNLLLPQGVVGQNITNLCPFGEVFNATRFASVYAWNRKRISNCVADYSV | 508 |
| | | | LYNSASFSTFKCYGVSPTKLNDLCFTNVYADSFVIRGDEVRQIAPGQTGKIADYNYKLPDDFTGCVIAW | |
| | | | NSNNLDSKVGGNYNYLRRLFRKSNLKPFERDISTEIYQAGSTPCNGVEGFNCYFPLQSYGFQPTNGV | |
| | | | GYQPYRVVVLSFENKSAPATVCGGGSGGSGNITNLCPFDEVFNATTFASVYAWNRKRISNCVADYSV | |
| | | | LYNFAPFFAFKCYGVSPTKLNDLCFTNVYADSFVIRGNEVSQIAPGQTGNIADYNYKLPDDFTGCVIAW | |
| | | | NSNKLDSTVGGNYNYRRRLFRKSKLKPFERDISTEIYQAGNKPCNGVAGVNCYFPLQSYGFRPTYGV | |
| | | | GHQPYRVVVLSFENKSAPATVCGGGSGGSGESQVRQQFSKDIEKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| | | BQ1.1-WA1 R-SpFN | MDSKGSSQKGSRLLLLLVVSNLLLPQGVVGQNITNLCPFDEVFNATTFASVYAWNRKRISNCVADYSV | 509 |
| | | | LYNFAPFFAFKCYGVSPTKLNDLCFTNVYADSFVIRGNEVSQIAPGQTGNIADYNYKLPDDFTGCVIAW | |
| | | | NSNKLDSTVGGNYNYRRRLFRKSKLKPFERDISTEIYQAGNKPCNGVAGVNCYFPLQSYGFRPTYGV | |
| | | | GHQPYRVVVLSFENKSAPATVCGGGSGGSGSSQCVNLTTRTQLPPAYTNSFTRGVYYPDKVFRSSV | |
| | | | LHSTQDLFLPFFSNVTWFHAIHVSGTNGTKRFDNPVLPFNDGVYFASTEKSNIIRGWIFGTTLDSKTQS | |
| | | | LLIVNNATNVVIKVCEFQFCNDPFLGVYYHKNNKSWMESEFRVYSSANNCTFEYVSQPFLMDLEGKQ | |
| | | | GNFKNLREFVFKNIDGYFKIYSKHTPINLVRDLPQGFSALEPLVDLPIGINITRFQTLLALHRSYLTPGDS | |
| | | | SSGWTAGAAAYYVGYLQPRTFLLKYNENGTITDAVDCALDPLSETKCTLKSFTVEKGIYQTSNFRVQP | |
| | | | TESIVRFPNITNLCPFGEVFNATRFASVYAWNRKRISNCVADYSVLYNSASFSTFKCYGVSPTKLNDLC | |
| | | | FTNVYADSFVIRGDEVRQIAPGQTGKIADYNYKLPDDFTGCVIAWNSNNLDSKVGGNYNYLYRLFRKS | |
| | | | NLKPFERDISTEIYQAGSTPCNGVEGFNCYFPLQSYGFQPTNGVGYQPYRVVVLSFELLHAPATVCGP | |
| | | | KKSTNLVKNKCVNFNFNGLTGTGVLTESNKKFLPFQQFGRDIADTTDAVRDPQTLEILDITPCSFGGVS | |
| | | | VITPGTNTSNQVAVLYQDVNCTEVPVAIHADQLTPTWRVYSTGSNVFQTRAGCLIGAEHVNNSYECDI | |
| | | | PIGAGICASYQTQTNSPGSASSVASQSIIAYTMSLGAENSVAYSNNSIAIPTNFTISVTTEILPVSMTKTS | |
| | | | VDCTMYICGDSTECSNLLLQYGSFCTQLNRALTGIAVEQDKNTQEVFAQVKQIYKTPPIKDFGGFNFS | |
| | | | QILPDPSKPSKRSFIEDLLFNKVTLADAGFIKQYGDCLGDIAARDLICAQKFNGLTVLPPLLTDEMIAQYT | |
| | | | SALLAGTITSGWTFGAGAALQIPFAMQMAYRFNGIGVTQNVLYENQKLIANQFNSAIGKIQDSLSSTAS | |
| | | | ALGKLQDVVNQNAQALNTLVKQLSSNFGAISSVLNDILSRLDPPEAEVQIDRLITGRLQSLQTYVTQQLI | |
| | | | RAAEIRASANLAATKMSECVLGQSKRVDFCGKGYHLMSFPQSAPHGVVFLHVTYVPAQEKNFTTAPAI | |
| | | | CHDGKAHFPREGVFVSNGTHWFVTQRNFYEPQIITTDNTFVSGNCDWVIGIVNNTVYDPLQSELDSIKE | |
| | | | ELDKIHKNGSGGSGDIIKLLNEQVNKEMQSSNLYMSMSSWCYTHSLDGAGLFLFDHAAEEYEHAKKLII | |
| | | | FLNENNVPVQLTSISAPEHKFEGLTQIFQKAYEHEQHISESINNIVDHAIKSKDHATFNFLQWYVAEQHE | |
| | | | EEVLFKDILDKIELIGNENHGLYLADQYVKGIAKSRKSGS | |
| | | BQ1.1 mRNA_S2P | MDSKGSSQKGSRLLLLLVVSNLLLPQGVVGQSSQCVNLITRTQSYTNSFTRGVYYPDKVFRSSVLHST | 510 |
| | | | QDLFLPFFSNVTWFHAISGTNGTKRFDNPVLPFNDGVYFASTEKSNIIRGWIFGTTLDSKTQSLLIVNNA | |
| | | | TNVVIKVCEFQFCNDPFLDVYYHKNNKSWMESEFRVYSSANNCTFEYVSQPFLMDLEGKQGNFKNLR | |
| | | | EFVFKNIDGYFKIYSKHTPINLGRDLPQGFSALEPLVDLPIGINITRFQTLLALHRSYLTPGDSSSGWTAG | |
| | | | AAAYYVGYLQPRTFLLKYNENGTITDAVDCALDPLSETKCTLKSFTVEKGIYQTSNFRVQPTESIVRFPN | |
| | | | ITNLCPFDEVFNATTFASVYAWNRKRISNCVADYSVLYNFAPFFAFKCYGVSPTKLNDLCFTNVYADSF | |
| | | | VIRGNEVSQIAPGQTGNIADYNYKLPDDFTGCVIAWNSNKLDSTVGGNYNYRYRLFRKSKLKPFERDIS | |
| | | | TEIYQAGNKPCNGVAGVNCYFPLQSYGFRPTYGVGHQPYRVVVLSFELLHAPATVCGPKKSTNLVKN | |
| | | | KCVNFNFNGLTGTGVLTESNKKFLPFQQFGRDIADTTDAVRDPQTLEILDITPCSFGGVSVITPGTNTS | |
| | | | NQVAVLYQGVNCTEVPVAIHADQLTPTWRVYSTGSNVFQTRAGCLIGAEYVNNSYECDIPIGAGICAS | |
| | | | YQTQTKSHGSASSVASQSIIAYTMSLGAENSVAYSNNSIAIPTNFTISVTTEILPVSMTKTSVDCTMYICG | |
| | | | DSTECSNLLLQYGSFCTQLKRALTGIAVEQDKNTQEVFAQVKQIYKTPPIKYFGGFNFSQILPDPSKPS | |
| | | | KRSFIEDLLFNKVTLADAGFIKQYGDCLGDIAARDLICAQKFNGLTVLPPLLTDEMIAQYTSALLAGTITS | |
| | | | GWTFGAGAALQIPFAMQMAYRFNGIGVTQNVLYENQKLIANQFNSAIGKIQDSLSSTASALGKLQDVV | |
| | | | NHNAQALNTLVKQLSSKFGAISSVLNDILSRLDPPEAEVQIDRLITGRLQSLQTYVTQQLIRAAEIRASAN | |
| | | | LAATKMSECVLGQSKRVDFCGKGYHLMSFPQSAPHGVVFLHVTYVPAQEKNFTTAPAICHDGKAHFP | |
| | | | REGVFVSNGTHWFVTQRNFYEPQIITTDNTFVSGNCDVVIGIVNNTVYDPLQPELDSFKEELDKYFKNH | |
| | | | TSPDVDLGDISGINASVVNIQKEIDRLNEVAKNLNESLIDLQELGKYEQYIKWPWYIWLGFIAGLIAIVMV | |
| | | | TIMLCCMTSCCSCLKGCCSCGSCCKFDEDDSEPVLKGVKLHY | |
| pCoV322 | | His8-3c-RR-SpFN where MERS | HHHHHHHHGPLEVLFQGPEGVECDFSPLLSGTPPQVYNFKRLVFTNCNYNLTKLLSLFSVNDFTCSQI | 511 |
| | | (M3) RBD is added in | SPAAIASNCYSSLILDYFSYPLSMKSDLSVSSAGPISQFNYKQSFSNPTCLILATVPHNLTTITKPLKYSYI | |
| | | addition to the SARS1 RBD | NKCSRLLSDDRTEVPQLVNANQYSPCVSIVPSTVWEDGDYYRKQLSPLEGGGWLVASGSTVAMTEQ | |
| | | (with 131like mutations) | LQMGFGITVQNGTDTNSVCPKGSGGSGNITNLCPFGEVFNATKFPSVYAWERKKISNCVADYSVLYN | |
| | | | STFFSTFKCYGVSATKLNDLCFSNVYADSFVVKGDDVRQIAPGQTGVIADYNYKLPDDFMGCVLAWN | |
| | | | TRNIDATSTGNYNYKRRYLRHGKLRPFERDISNVPFSPDGKPCTPPALNCYWPLNDYGFYTTTGIGYQ | |
| | | | PYRVVVLSFENKSAPATVCGPGSGGSGSSQCVNLTTRTQLPPAYTNSFTRGVYYPDKVFRSSVLHST | |
| | | | QDLFLPFFSNVTWFHAIHVSGTNGTKRFDNPVLPFNDGVYFASTEKSNIIRGWIFGTTLDSKTQSLLIVN | |
| | | | NATNVVIKVCEFQFCNDPFLGVYYHKNNKSWMESEFRVYSSANNCTFEYVSQPFLMDLEGKQGNFK | |
| | | | NLREFVFKNIDGYFKIYSKHTPINLVRDLPQGFSALEPLVDLPIGINITRFQTLLALHRSYLTPGDSSSGW | |
| | | | TAGAAAYYVGYLQPRTFLLKYNENGTITDAVDCALDPLSETKCTLKSFTVEKGIYQTSNFRVQPTESIV | |
| | | | RFPNITNLCPFGEVFNATRFASVYAWNRKRISNCVADYSVLYNSASFSTFKCYGVSPTKLNDLCFTNV | |
| | | | YADSFVIRGDEVRQIAPGQTGKIADYNYKLPDDFTGCVIAWNSNNLDSKVGGNYNYLYRLFRKSNLKP | |
| | | | FERDISTEIYQAGSTPCNGVEGFNCYFPLQSYGFQPTNGVGYQPYRVVVLSFELLHAPATVCGPKKST | |
| | | | NLVKNKCVNFNFNGLTGTGVLTESNKKFLPFQQFGRDIADTTDAVRDPQTLEILDITPCSFGGVSVITP | |
| | | | GTNTSNQVAVLYQDVNCTEVPVAIHADQLTPTWRVYSTGSNVFQTRAGCLIGAEHVNNSYECDIPIGA | |
| | | | GICASYQTQTNSPGSASSVASQSIIAYTMSLGAENSVAYSNNSIAIPTNFTISVTTEILPVSMTKTSVDCT | |
| | | | MYICGDSTECSNLLLQYGSFCTQLNRALTGIAVEQDKNTQEVFAQVKQIYKTPPIKDFGGFNFSQILPD | |
| | | | PSKPSKRSFIEDLLFNKVTLADAGFIKQYGDCLGDIAARDLICAQKFNGLTVLPPLLTDEMIAQYTSALLA | |
| | | | GTITSGWTFGAGAALQIPFAMQMAYRFNGIGVTQNVLYENQKLIANQFNSAIGKIQDSLSSTASALGKL | |
| | | | QDVVNQNAQALNTLVKQLSSNFGAISSVLNDILSRLDPPEAEVQIDRLITGRLQSLQTYVTQQLIRAAEI | |
| | | | RASANLAATKMSECVLGQSKRVDFCGKGYHLMSFPQSAPHGVVFLHVTYVPAQEKNFTTAPAICHDG | |
| | | | KAHFPREGVFVSNGTHWFVTQRNFYEPQIITTDNTFVSGNCDVVIGIVNNTVYDPLQSELDSIKEELDKI | |
| | | | HKNGSGGSGDIIKLLNEQVNKEMQSSNLYMSMSSWCYTHSLDGAGLFLFDHAAEEYEHAKKLIIFLNE | |
| | | | NNVPVQLTSISAPEHKFEGLTQIFQKAYEHEQHISESINNIVDHAIKSKDHATFNFLQWYVAEQHEEEVL | |
| | | | FKDILDKIELIGNENHGLYLADQYVKGIAKSRKSGS | |
| pCoV323 | | His8-3c-RR-SpFN where MR14 | HHHHHHHHGPLEVLFQGPNITNLCPFSQVFNATRFPSVYAWTRERISNCIADYSVLYNSTSFSTFRCY | 512 |
| | | is added in addition to the | GVSPTKLNDLCFSNVYADSMVVRGDEVRQIAPSQTGVIADYNYKLPDDFTGCVIAWNSKAKDENGQY | |
| | | SARS1 RBD (with 131like | FYRLFRKSKLLPFQRDVSNVTYGSGKNDGCNPSEADCYWPLLKYGFTSSVSQDYQPYRVVVLSFELL | |
| | | mutations) | NAPATVCGPGSGGSGNITNLCPFGEVFNATKFPSVYAWERKKISNCVADYSVLYNSTFFSTFKCYGVS | |
| | | | ATKLNDLCFSNVYADSFWKGDDVRQIAPGQTGVIADYNYKLPDDFMGCVLAWNTRNIDATSTGNYN | |
| | | | YKRRYLRHGKLRPFERDISNVPFSPDGKPCTPPALNCYWPLNDYGFYTTTGIGYQPYRVVVLSFENKS | |
| | | | APATVCGPGSGGSGSSQCVNLTTRTQLPPAYTNSFTRGVYYPDKVFRSSVLHSTQDLFLPFFSNVTW | |
| | | | FHAIHVSGTNGTKRFDNPVLPFNDGVYFASTEKSNIIRGWIFGTTLDSKTQSLLIVNNATNVVIKVCEFQ | |
| | | | FCNDPFLGVYYHKNNKSWMESEFRVYSSANNCTFEYVSQPFLMDLEGKQGNFKNLREFVFKNIDGYF | |
| | | | KIYSKHTPINLVRDLPQGFSALEPLVDLPIGINITRFQTLLALHRSYLTPGDSSSGWTAGAAAYYVGYLQ | |
| | | | PRTFLLKYNENGTITDAVDCALDPLSETKCTLKSFTVEKGIYQTSNFRVQPTESIVRFPNITNLCPFGEV | |
| | | | FNATRFASVYAWNRKRISNCVADYSVLYNSASFSTFKCYGVSPTKLNDLCFTNVYADSFVIRGDEVRQ | |
| | | | IAPGQTGKIADYNYKLPDDFTGCVIAWNSNNLDSKVGGNYNYLYRLFRKSNLKPFERDISTEIYQAGST | |
| | | | PCNGVEGFNCYFPLQSYGFQPTNGVGYQPYRVWVLSFELLHAPATVCGPKKSTNLVKNKCVNFNFNG | |
| | | | LTGTGVLTESNKKFLPFQQFGRDIADTTDAVRDPQTLEILDITPCSFGGVSVITPGTNTSNQVAVLYQD | |
| | | | VNCTEVPVAIHADQLTPTWRVYSTGSNVFQTRAGCLIGAEHVNNSYECDIPIGAGICASYQTQTNSPG | |
| | | | SASSVASQSIIAYTMSLGAENSVAYSNNSIAIPTNFTISVTTEILPVSMTKTSVDCTMYICGDSTECSNLL | |
| | | | LQYGSFCTQLNRALTGIAVEQDKNTQEVFAQVKQIYKTPPIKDFGGFNFSQILPDPSKPSKRSFIEDLLF | |
| | | | NKVTLADAGFIKQYGDCLGDIAARDLICAQKFNGLTVLPPLLTDEMIAQYTSALLAGTITSGWTFGAGAA | |
| | | | LQIPFAMQMAYRFNGIGVTQNVLYENQKLIANQFNSAIGKIQDSLSSTASALGKLQDVVNQNAQALNTL | |
| | | | VKQLSSNFGAISSVLNDILSRLDPPEAEVQIDRLITGRLQSLQTYVTQQLIRAAEIRASANLAATKMSECV | |
| | | | LGQSKRVDFCGKGYHLMSFPQSAPHGVVFLHVTYVPAQEKNFTTAPAICHDGKAHFPREGVFVSNGT | |
| | | | HWFVTQRNFYEPQIITTDNTFVSGNCDWVIGIVNNTVYDPLQSELDSIKEELDKIHKNGSGGSGDIIKLLN | |
| | | | EQVNKEMQSSNLYMSMSSWCYTHSLDGAGLFLFDHAAEEYEHAKKLIIFLNENNVPVQLTSISAPEHK | |
| | | | FEGLTQIFQKAYEHEQHISESINNIVDHAIKSKDHATFNFLQWYVAEQHEEEVLFKDILDKIELIGNENHG | |
| | | | LYLADQYVKGIAKSRKSGS | |
| pCoV326 | | His8-3c-RR-SpFN where MR15 | HHHHHHHHGPLEVLFQGPNMTNICPFDQVFNKTQFPSVYAWERVRISDCVSDYTVLYNSSASFSTFK | 513 |
| | | is added in addition to the | CYGVSPTKLNDLCFSGVYADYFVVKGDHVHQIAPGQTGVIADYNYKLPSEFVGCILAWNTRTIDSKRG | |
| | | SARS1 RBD (with 131like | FYYRLFRHGNIRPYERDTSNVPYNAAGGTCNQPGTHNCYEPLQDYGFTSTSGVGYQPFRVVVLSFEL | |
| | | mutations) | LNAPATVCGPGSGGSGNITNLCPFGEVFNATKFPSVYAWERKKISNCVADYSVLYNSTFFSTFKCYGV | |
| | | | SATKLNDLCFSNVYADSFVVKGDDVRQIAPGQTGVIADYNYKLPDDFMGCVLAWNTRNIDATSTGNY | |
| | | | NYKRRYLRHGKLRPFERDISNVPFSPDGKPCTPPALNCYWPLNDYGFYTTTGIGYQPYRVVVLSFENK | |
| | | | SAPATVCGPGSGGSGSSQCVNLTTRTQLPPAYTNSFTRGVYYPDKVFRSSVLHSTQDLFLPFFSNVT | |
| | | | WFHAIHVSGTNGTKRFDNPVLPFNDGVYFASTEKSNIIRGWIFGTTLDSKTQSLLIVNNATNVVIKVCEF | |
| | | | QFCNDPFLGVYYHKNNKSWMESEFRVYSSANNCTFEYVSQPFLMDLEGKQGNFKNLREFVFKNIDG | |
| | | | YFKIYSKHTPINLVRDLPQGFSALEPLVDLPIGINITRFQTLLALHRSYLTPGDSSSGWTAGAAAYYVGY | |
| | | | LQPRTFLLKYNENGTITDAVDCALDPLSETKCTLKSFTVEKGIYQTSNFRVQPTESIVRFPNITNLCPFG | |
| | | | EVFNATRFASVYAWNRKRISNCVADYSVLYNSASFSTFKCYGVSPTKLNDLCFTNVYADSFVIRGDEV | |
| | | | RQIAPGQTGKIADYNYKLPDDFTGCVIAWNSNNLDSKVGGNYNYLYRLFRKSNLKPFERDISTEIYQAG | |
| | | | STPCNGVEGFNCYFPLQSYGFQPTNGVGYQPYRVVVLSFELLHAPATVCGPKKSTNLVKNKCVNFNF | |
| | | | NGLTGTGVLTESNKKFLPFQQFGRDIADTTDAVRDPQTLEILDITPCSFGGVSVITPGTNTSNQVAVLY | |
| | | | QDVNCTEVPVAIHADQLTPTWRVYSTGSNVFQTRAGCLIGAEHVNNSYECDIPIGAGICASYQTQTNS | |
| | | | PGSASSVASQSIIAYTMSLGAENSVAYSNNSIAIPTNFTISVTTEILPVSMTKTSVDCTMYICGDSTECSN | |
| | | | LLLQYGSFCTQLNRALTGIAVEQDKNTQEVFAQVKQIYKTPPIKDFGGFNFSQILPDPSKPSKRSFIEDL | |
| | | | LFNKVTLADAGFIKQYGDCLGDIAARDLICAQKFNGLTVLPPLLTDEMIAQYTSALLAGTITSGWTFGAG | |
| | | | AALQIPFAMQMAYRFNGIGVTQNVLYENQKLIANQFNSAIGKIQDSLSSTASALGKLQDVVNQNAQALN | |
| | | | TLVKQLSSNFGAISSVLNDILSRLDPPEAEVQIDRLITGRLQSLQTYVTQQLIRAAEIRASANLAATKMSE | |
| | | | CVLGQSKRVDFCGKGYHLMSFPQSAPHGVVFLHVTYVPAQEKNFTTAPAICHDGKAHFPREGVFVSN | |
| | | | GTHWFVTQRNFYEPQIITTDNTFVSGNCDVVIGIVNNTVYDPLQSELDSIKEELDKIHKNGSGGSGDIIK | |
| | | | LLNEQVNKEMQSSNLYMSMSSWCYTHSLDGAGLFLFDHAAEEYEHAKKLIIFLNENNVPVQLTSISAPE | |
| | | | HKFEGLTQIFQKAYEHEQHISESINNIVDHAIKSKDHATFNFLQWYVAEQHEEEVLFKDILDKIELIGNEN | |
| | | | HGLYLADQYVKGIAKSRKSGS | |
| pCoV327 | | His8-3c-RR-SpFN where MR08 | HHHHHHHHGPLEVLFQGPNITNFCPFDKVFNATRFPNVYAWQRTKISDCIADYTVLYNSTSFSTFKCY | 514 |
| | | is added in addition to the | GVSPSKLIDLCFTSVYADTFLIRFSEVRQIAPGETGVIADYNYKLPDDFTGCVLAWNTAQQDIGSYFYR | |
| | | SARS1 RBD (with 131like | SHRAVKLKPFERDLSSDENGVRTLSTYDFNPNVPLDYQATRVVVLSFELLNAPATVCGPGSGGSGNIT | |
| | | mutations) | NLCPFGEVFNATKFPSVYAWERKKISNCVADYSVLYNSTFFSTFKCYGVSATKLNDLCFSNVYADSFV | |
| | | | VKGDDVRQIAPGQTGVIADYNYKLPDDFMGCVLAWNTRNIDATSTGNYNYKRRYLRHGKLRPFERDIS | |
| | | | NVPFSPDGKPCTPPALNCYWPLNDYGFYTTTGIGYQPYRVVVLSFENKSAPATVCGPGSGGSGSSQ | |
| | | | CVNLTTRTQLPPAYTNSFTRGVYYPDKVFRSSVLHSTQDLFLPFFSNVTWFHAIHVSGTNGTKRFDNP | |
| | | | VLPFNDGVYFASTEKSNIIRGWIFGTTLDSKTQSLLIVNNATNVVIKVCEFQFCNDPFLGVYYHKNNKS | |
| | | | WMESEFRVYSSANNCTFEYVSQPFLMDLEGKQGNFKNLREFVFKNIDGYFKIYSKHTPINLVRDLPQG | |
| | | | FSALEPLVDLPIGINITRFQTLLALHRSYLTPGDSSSGWTAGAAAYYVGYLQPRTFLLKYNENGTITDAV | |
| | | | DCALDPLSETKCTLKSFTVEKGIYQTSNFRVQPTESIVRFPNITNLCPFGEVFNATRFASVYAWNRKRI | |
| | | | SNCVADYSVLYNSASFSTFKCYGVSPTKLNDLCFTNVYADSFVIRGDEVRQIAPGQTGKIADYNYKLPD | |
| | | | DFTGCVIAWNSNNLDSKVGGNYNYLYRLFRKSNLKPFERDISTEIYQAGSTPCNGVEGFNCYFPLQSY | |
| | | | GFQPTNGVGYQPYRVVVLSFELLHAPATVCGPKKSTNLVKNKCVNFNFNGLTGTGVLTESNKKFLPF | |
| | | | QQFGRDIADTTDAVRDPQTLEILDITPCSFGGVSVITPGTNTSNQVAVLYQDVNCTEVPVAIHADQLTP | |
| | | | TWRVYSTGSNVFQTRAGCLIGAEHVNNSYECDIPIGAGICASYQTQTNSPGSASSVASQSIIAYTMSLG | |
| | | | AENSVAYSNNSIAIPTNFTISVTTEILPVSMTKTSVDCTMYICGDSTECSNLLLQYGSFCTQLNRALTGIA | |
| | | | VEQDKNTQEVFAQVKQIYKTPPIKDFGGFNFSQILPDPSKPSKRSFIEDLLFNKVTLADAGFIKQYGDCL | |
| | | | GDIAARDLICAQKFNGLTVLPPLLTDEMIAQYTSALLAGTITSGWTFGAGAALQIPFAMQMAYRFNGIG | |
| | | | VTQNVLYENQKLIANQFNSAIGKIQDSLSSTASALGKLQDVVNQNAQALNTLVKQLSSNFGAISSVLNDI | |
| | | | LSRLDPPEAEVQIDRLITGRLQSLQTYVTQQLIRAAEIRASANLAATKMSECVLGQSKRVDFCGKGYHL | |
| | | | MSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMS | |
| | | | MSSWCYTHSLDGAGLFLFDHAAEEYEHAKKLIIFLNENNVPVQLTSISAPEHKFEGLTQIFQKAYEHEQ | |
| | | | HISESINNIVDHAIKSKDHATFNFLQWYVAEQHEEEVLFKDILDKIELIGNENHGLYLADQYVKGIAKSRK | |
| | | | SGS | |
| pCoV328 | | His8-3c-RR-SpFN where MR14 | HHHHHHHHGPLEVLFQGPNITNLCPFSQVFNATRFPSVYAWTRERISNCIADYSVLYNSTSFSTFRCY | 515 |
| | | and MR15 are added | GVSPTKLNDLCFSNVYADSMVVRGDEVRQIAPSQTGVIADYNYKLPDDFTGCVIAWNSKAKDENGQY | |
| | | | FYRLFRKSKLLPFQRDVSNVTYGSGKNDGCNPSEADCYWPLLKYGFTSSVSQDYQPYRVVVLSFELL | |
| | | | NAPATVCGPGSGGSGNMTNICPFDQVFNKTQFPSVYAWERVRISDCVSDYTVLYNSSASFSTFKCYG | |
| | | | VSPTKLNDLCFSGVYADYFWVKGDHVHQIAPGQTGVIADYNYKLPSEFVGCILAWNTRTIDSKRGFYY | |
| | | | RLFRHGNIRPYERDTSNVPYNAAGGTCNQPGTHNCYEPLQDYGFTSTSGVGYQPFRVVVLSFELLNA | |
| | | | PATVCGPGSGGSGSSQCVNLTTRTQLPPAYTNSFTRGVYYPDKVFRSSVLHSTQDLFLPFFSNVTWF | |
| | | | HAIHVSGTNGTKRFDNPVLPFNDGVYFASTEKSNIIRGWIFGTTLDSKTQSLLIVNNATNVVIKVCEFQF | |
| | | | CNDPFLGVYYHKNNKSWMESEFRVYSSANNCTFEYVSQPFLMDLEGKQGNFKNLREFVFKNIDGYFK | |
| | | | IYSKHTPINLVRDLPQGFSALEPLVDLPIGINITRFQTLLALHRSYLTPGDSSSGWTAGAAAYYVGYLQP | |
| | | | RTFLLKYNENGTITDAVDCALDPLSETKCTLKSFTVEKGIYQTSNFRVQPTESIVRFPNITNLCPFGEVF | |
| | | | NATRFASVYAWNRKRISNCVADYSVLYNSASFSTFKCYGVSPTKLNDLCFTNVYADSFVIRGDEVRQI | |
| | | | APGQTGKIADYNYKLPDDFTGCVIAWNSNNLDSKVGGNYNYLYRLFRKSNLKPFERDISTEIYQAGST | |
| | | | PCNGVEGFNCYFPLQSYGFQPTNGVGYQPYRVVVLSFELLHAPATVCGPKKSTNLVKNKCVNFNFNG | |
| | | | LTGTGVLTESNKKFLPFQQFGRDIADTTDAVRDPQTLEILDITPCSFGGVSVITPGTNTSNQVAVLYQD | |
| | | | VNCTEVPVAIHADQLTPTWRVYSTGSNVFQTRAGCLIGAEHVNNSYECDIPIGAGICASYQTQTNSPG | |
| | | | SASSVASQSIIAYTMSLGAENSVAYSNNSIAIPTNFTISVTTEILPVSMTKTSVDCTMYICGDSTECSNLL | |
| | | | LQYGSFCTQLNRALTGIAVEQDKNTQEVFAQVKQIYKTPPIKDFGGFNFSQILPDPSKPSKRSFIEDLLF | |
| | | | NKVTLADAGFIKQYGDCLGDIAARDLICAQKFNGLTVLPPLLTDEMIAQYTSALLAGTITSGWTFGAGAA | |
| | | | LQIPFAMQMAYRFNGIGVTQNVLYENQKLIANQFNSAIGKIQDSLSSTASALGKLQDVVNQNAQALNTL | |
| | | | VKQLSSNFGAISSVLNDILSRLDPPEAEVQIDRLITGRLQSLQTYVTQQLIRAAEIRASANLAATKMSECV | |
| | | | LGQSKRVDFCGKGYHLMSFPQSAPHGVVFLHVTYVPAQEKNFTTAPAICHDGKAHFPREGVFVSNGT | |
| | | | HWFVTQRNFYEPQIITTDNTFVSGNCDVVIGIVNNTVYDPLQSELDSIKEELDKIHKNGSGGSGDIIKLLN | |
| | | | EQVNKEMQSSNLYMSMSSWCYTHSLDGAGLFLFDHAAEEYEHAKKLIIFLNENNVPVQLTSISAPEHK | |
| | | | FEGLTQIFQKAYEHEQHISESINNIVDHAIKSKDHATFNFLQWYVAEQHEEEVLFKDILDKIELIGNENHG | |
| | | | LYLADQYVKGIAKSRKSGS | |
| pCoV329 | | His8-3c-RR-SpFN where MR14 | HHHHHHHHGPLEVLFQGPNITNLCPFSQVFNATRFPSVYAWTRERISNCIADYSVLYNSTSFSTFRCY | 516 |
| | | and MR08 are added | GVSPTKLNDLCFSNVYADSMVVRGDEVRQIAPSQTGVIADYNYKLPDDFTGCVIAWNSKAKDENGQY | |
| | | | FYRLFRKSKLLPFQRDVSNVTYGSGKNDGCNPSEADCYWPLLKYGFTSSVSQDYQPYRVVVLSFELL | |
| | | | NAPATVCGPGSGGSGNITNFCPFDKVFNATRFPNVYAWQRTKISDCIADYTVLYNSTSFSTFKCYGVS | |
| | | | PSKLIDLCFTSVYADTFLIRFSEVRQIAPGETGVIADYNYKLPDDFTGCVLAWNTAQQDIGSYFYRSHR | |
| | | | AVKLKPFERDLSSDENGVRTLSTYDFNPNVPLDYQATRVVVLSFELLNAPATVCGPGSGGSGSSQCV | |
| | | | NLTTRTQLPPAYTNSFTRGVYYPDKVFRSSVLHSTQDLFLPFFSNVTWFHAIHVSGTNGTKRFDNPVL | |
| | | | PFNDGVYFASTEKSNIIRGWIFGTTLDSKTQSLLIVNNATNVVIKVCEFQFCNDPFLGVYYHKNNKSWM | |
| | | | ESEFRVYSSANNCTFEYVSQPFLMDLEGKQGNFKNLREFVFKNIDGYFKIYSKHTPINLVRDLPQGFSA | |
| | | | LEPLVDLPIGINITRFQTLLALHRSYLTPGDSSSGWTAGAAAYYVGYLQPRTFLLKYNENGTITDAVDCA | |
| | | | LDPLSETKCTLKSFTVEKGIYQTSNFRVQPTESIVRFPNITNLCPFGEVFNATRFASVYAWNRKRISNC | |
| | | | VADYSVLYNSASFSTFKCYGVSPTKLNDLCFTNVYADSFVIRGDEVRQIAPGQTGKIADYNYKLPDDFT | |
| | | | GCVIAWNSNNLDSKVGGNYNYLYRLFRKSNLKPFERDISTEIYQAGSTPCNGVEGFNCYFPLQSYGFQ | |
| | | | PTNGVGYQPYRVVVLSFELLHAPATVCGPKKSTNLVKNKCVNFNFNGLTGTGVLTESNKKFLPFQQF | |
| | | | GRDIADTTDAVRDPQTLEILDITPCSFGGVSVITPGTNTSNQVAVLYQDVNCTEVPVAIHADQLTPTWR | |
| | | | VYSTGSNVFQTRAGCLIGAEHVNNSYECDIPIGAGICASYQTQTNSPGSASSVASQSIIAYTMSLGAEN | |
| | | | SVAYSNNSIAIPTNFTISVTTEILPVSMTKTSVDCTMYICGDSTECSNLLLQYGSFCTQLNRALTGIAVEQ | |
| | | | DKNTQEVFAQVKQIYKTPPIKDFGGFNFSQILPDPSKPSKRSFIEDLLFNKVTLADAGFIKQYGDCLGDI | |
| | | | AARDLICAQKFNGLTVLPPLLTDEMIAQYTSALLAGTITSGWTFGAGAALQIPFAMQMAYRFNGIGVTQ | |
| | | | NVLYENQKLIANQFNSAIGKIQDSLSSTASALGKLQDVVNQNAQALNTLVKQLSSNFGAISSVLNDILSR | |
| | | | LDPPEAEVQIDRLITGRLQSLQTYVTQQLIRAAEIRASANLAATKMSECVLGQSKRVDFCGKGYHLMSF | |
| | | | PQSAPHGVVFLHVTYVPAQEKNFTTAPAICHDGKAHFPREGVFVSNGTHWFVTQRNFYEPQIITTDNT | |
| | | | FVSGNCDVVIGIVNNTVYDPLQSELDSIKEELDKIHKNGSGGSGDIIKLLNEQVNKEMQSSNLYMSMSS | |
| | | | WCYTHSLDGAGLFLFDHAAEEYEHAKKLIIFLNENNVPVQLTSISAPEHKFEGLTQIFQKAYEHEQHISE | |
| | | | SINNIVDHAIKSKDHATFNFLQWYVAEQHEEEVLFKDILDKIELIGNENHGLYLADQYVKGIAKSRKSGS | |
| pCoV330 | | His8-3c-RRR-SpFN where MR15 | HIHHHHGPLEVLFQGPNMTNICPFDQVFNKTQFPSVYAWERVRISDCVSDYTVLYNSSASFSTFKCYG | 517 |
| | | is added in addition to | VSPTKLNDLCFSGVYADYFVVKGDHVHQIAPGQTGVIADYNYKLPSEFVGCILAWNTRTIDSKRGFYYRLFR | |
| | | the MR14 and SARS1 RBD | HGNIRPYERDTSNVPYNAAGGTCNQPGTHNCYEPLQDYGFTSTSGVGYQPFRWVVLSFELLNAPATVCGP | |
| | | (with 131like mutations) | GSGGSGNITNLCPFSQVFNATRFPSVYAWTRERISNCIADYSVLYNSTSFSTFRCYGVSPTKLNDLCFSNVY | |
| | | | ADSMVVRGDEVRQIAPSQTGVIADYNYKLPDDFTGCVIAWNSKAKDENGQYFYRLFRKSKLLPFQRDVSNV | |
| | | | TYGSGKNDGCNPSEADCYWPLLKYGFTSSVSQDYQPYRVVVLSFELLNAPATVCGPGSGGSGNITNLCPF | |
| | | | GEVFNATKFPSVYAWERKKISNCVADYSVLYNSTFFSTFKCYGVSATKLNDLCFSNVYADSFVVKGDDVRQ | |
| | | | IAPGQTGVIADYNYKLPDDFMGCVLAWNTRNIDATSTGNYNYKRRYLRHGKLRPFERDISNVPFSPDGKPC | |
| | | | TPPALNCYWPLNDYGFYTTTGIGYQPYRVVVLSFENKSAPATVCGPGSGGSGSSQCVNLTTRTQLPPAYT | |
| | | | NSFTRGVYYPDKVFRSSVLHSTQDLFLPFFSNVTWFHAIHVSGTNGTKRFDNPVLPFNDGVYFASTEKSNII | |
| | | | RGWIFGTTLDSKTQSLLIVNNATNVVIKVCEFQFCNDPFLGVYYHKNNKSWMESEFRVYSSANNCTFEYVS | |
| | | | QPFLMDLEGKQGNFKNLREFVFKNIDGYFKIYSKHTPINLVRDLPQGFSALEPLVDLPIGINITRFQTLLALH | |
| | | | RSYLTPGDSSSGWTAGAAAYYVGYLQPRTFLLKYNENGTITDAVDCALDPLSETKCTLKSFTVEKGIYQTSNF | |
| | | | RVQPTESIVRFPNITNLCPFGEVFNATRFASVYAWNRKRISNCVADYSVLYNSASFSTFKCYGVSPTKLNDLC | |
| | | | FTNVYADSFVIRGDEVRQIAPGQTGKIADYNYKLPDDFTGCVIAWNSNNLDSKVGGNYNYLYRLFRKSNLKP | |
| | | | FERDISTEIYQAGSTPCNGVEGFNCYFPLQSYGFQPTNGVGYQPYRVVVLSFELLHAPATVCGPKKSTNLV | |
| | | | KNKCVNFNFNGLTGTGVLTESNKKFLPFQQFGRDIADTTDAVRDPQTLEILDITPCSFGGVSVITPGTNTSNQ | |
| | | | VAVLYQDVNCTEVPVAIHADQLTPTWRVYSTGSNVFQTRAGCLIGAEHVNNSYECDIPIGAGICASYQTQTN | |
| | | | SPGSASSVASQSIIAYTMSLGAENSVAYSNNSIAIPTNFTISVTTEILPVSMTKTSVDCTMYICGDSTECSNL | |
| | | | LLQYGSFCTQLNRALTGIAVEQDKNTQEVFAQVKQIYKTPPIKDFGGFNFSQILPDPSKPSKRSFIEDLLFNK | |
| | | | VTLADAGFIKQYGDCLGDIAARDLICAQKFNGLTVLPPLLTDEMIAQYTSALLAGTITSGWTFGAGAALQIPF | |
| | | | AMQMAYRFNGIGVTQNVLYENQKLIANQFNSAIGKIQDSLSSTASALGKLQDVVNQNAQALNTLVKQLSSNFG | |
| | | | AISSVLNDILSRLDPPEAEVQIDRLITGRLQSLQTYVTQQLIRAAEIRASANLAATKMSECVLGQSKRVDFCG | |
| | | | KGYHLMSFPQSAPHGVVFLHVTYVPAQEKNFTTAPAICHDGKAHFPREGVFVSNGTHWFVTQRNFYEPQIITT | |
| | | | DNTFVSGNCDVVIGIVNNTVYDPLQSELDSIKEELDKIHKNGSGGSGDIIKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESINN | |
| | | | IVDHAIKSKDHATFNFLQWYVAEQHEEEVLFKDILDKIELIGNENHGLYLADQYVKGIAKSRKSGS | |
| pCoV331 | | His8-3c-RRR-SpFN where MR08 | HHHHHHHHGPLEVLFQGPNITNFCPFDKVFNATRFPNVYAWQRTKISDCIADYTVLYNSTSFSTFKCY | 518 |
| | | is added in addition t | GVSPSKLIDLCFTSVYADTFLIRFSEVRQIAPGETGVIADYNYKLPDDFTGCVLAWNTAQQDIGSYFYR | |
| | | the MR14 and SARS1 RBD | SHRAVKLKPFERDLSSDENGVRTLSTYDFNPNVPLDYQATRVVVLSFELLNAPATVCGPGSGGSGNIT | |
| | | (with 131like mutations) | NLCPFSQVFNATRFPSVYAWTRERISNCIADYSVLYNSTSFSTFRCYGVSPTKLNDLCFSNVYADSMV | |
| | | | VRGDEVRQIAPSQTGVIADYNYKLPDDFTGCVIAWNSKAKDENGQYFYRLFRKSKLLPFQRDVSNVTY | |
| | | | GSGKNDGCNPSEADCYWPLLKYGFTSSVSQDYQPYRVVVLSFELLNAPATVCGPGSGGSGNITNLC | |
| | | | PFGEVFNATKFPSVYAWERKKISNCVADYSVLYNSTFFSTFKCYGVSATKLNDLCFSNVYADSFVVKG | |
| | | | DDVRQIAPGQTGVIADYNYKLPDDFMGCVLAWNTRNIDATSTGNYNYKRRYLRHGKLRPFERDISNVP | |
| | | | FSPDGKPCTPPALNCYWPLNDYGFYTTTGIGYQPYRVVVLSFENKSAPATVCGPGSGGSGSSQCVN | |
| | | | LTTRTQLPPAYTNSFTRGVYYPDKVFRSSVLHSTQDLFLPFFSNVTWFHAIHVSGTNGTKRFDNPVLP | |
| | | | FNDGVYFASTEKSNIIRGWIFGTTLDSKTQSLLIVNNATNVVIKVCEFQFCNDPFLGVYYHKNNKSWME | |
| | | | SEFRVYSSANNCTFEYVSQPFLMDLEGKQGNFKNLREFVFKNIDGYFKIYSKHTPINLVRDLPQGFSAL | |
| | | | EPLVDLPIGINITRFQTLLALHRSYLTPGDSSSGWTAGAAAYYVGYLQPRTFLLKYNENGTITDAVDCAL | |
| | | | DPLSETKCTLKSFTVEKGIYQTSNFRVQPTESIVRFPNITNLCPFGEVFNATRFASVYAWNRKRISNCV | |
| | | | ADYSVLYNSASFSTFKCYGVSPTKLNDLCFTNVYADSFVIRGDEVRQIAPGQTGKIADYNYKLPDDFTG | |
| | | | CVIAWNSNNLDSKVGGNYNYLYRLFRKSNLKPFERDISTEIYQAGSTPCNGVEGFNCYFPLQSYGFQP | |
| | | | TNGVGYQPYRVVVLSFELLHAPATVCGPKKSTNLVKNKCVNFNFNGLTGTGVLTESNKKFLPFQQFG | |
| | | | RDIADTTDAVRDPQTLEILDITPCSFGGVSVITPGTNTSNQVAVLYQDVNCTEVPVAIHADQLTPTWRV | |
| | | | YSTGSNVFQTRAGCLIGAEHVNNSYECDIPIGAGICASYQTQTNSPGSASSVASQSIIAYTMSLGAENS | |
| | | | VAYSNNSIAIPTNFTISVTTEILPVSMTKTSVDCTMYICGDSTECSNLLLQYGSFCTQLNRALTGIAVEQD | |
| | | | KNTQEVFAQVKQIYKTPPIKDFGGFNFSQILPDPSKPSKRSFIEDLLFNKVTLADAGFIKQYGDCLGDIA | |
| | | | ARDLICAQKFNGLTVLPPLLTDEMIAQYTSALLAGTITSGWTFGAGAALQIPFAMQMAYRFNGIGVTQN | |
| | | | VLYENQKLIANQFNSAIGKIQDSLSSTASALGKLQDVVNQNAQALNTLVKQLSSNFGAISSVLNDILSRL | |
| | | | DPPEAEVQIDRLITGRLQSLQTYVTQQLIRAAEIRASANLAATKMSECVLGQSKRVDFCGKGYHLMSFP | |
| | | | QSAPHGVVFLHVTYVPAQEKNFTTAPAICHDGKAHFPREGVFVSNGTHWFVTQRNFYEPQIITTDNTF | |
| | | | VSGNCDWVIGIVNNTVYDPLQSELDSIKEELDKIHKNGSGGSGDIIKLLNEQVNKEMQSSNLYMSMSSW | |
| | | | CYTHSLDGAGLFLFDHAAEEYEHAKKLIIFLNENNVPVQLTSISAPEHKFEGLTQIFQKAYEHEQHISESI | |
| | | | NNIVDHAIKSKDHATFNFLQWYVAEQHEEEVLFKDILDKIELIGNENHGLYLADQYVKGIAKSRKSGS | |
| pCoV332 | | His8-3c-RR-SpFN-loop2 | HHHHHHHHGPLEVLFQGPNITNLCPFSQVFNATRFPSVYAWTRERISNCIADYSVLYNSTSFSTFRCYGVSP | 519 |
| | | where MR14 and | TKLNDLCFSNVYADSMVVRGDEVRQIAPSQTGVIADYNYKLPDDFTGCVIAWNSKAKDENGQYFYRLFRKS | |
| | | SARS1(131like) RBD at N- | KLLPFQRDVSNVTYGSGKNDGCNPSEADCYWPLLKYGFTSSVSQDYQPYRVVVLSFELLNAPATVCGPGS | |
| | | term and MR15 and MR08 are | GGSGNITNLCPFGEVFNATKFPSVYAWERKKISNCVADYSVLYNSTFFSTFKCYGVSATKLNDLCFSNVYAD | |
| | | inserted at NTD loops | SFVVKGDDVRQIAPGQTGVIADYNYKLPDDFMGCVLAWNTRNIDATSTGNYNYKRRYLRHGKLRPFERDIS | |
| | | | NVPFSPDGKPCTPPALNCYWPLNDYGFYTTTGIGYQPYRVVVLSFENKSAPATVCGPGSGGSGSSQCVNL | |
| | | | TTRTQLPPAYTNSFTRGVYYPDKVFRSSVLHSTQDLFLPFFSNVTWFHAIHVGSGGSGNMTNICPFDQVEN | |
| | | | KTQFPSVYAWERVRISDCVSDYTVLYNSSASFSTFKCYGVSPTKLNDLCFSGVYADYFVVKGDHVHQIAPG | |
| | | | QTGVIADYNYKLPSEFVGCILAWNTRTIDSKRGFYYRLFRHGNIRPYERDTSNVPYNAAGGTCNQPGTHNC | |
| | | | YEPLQDYGFTSTSGVGYQPFRVVVLSFELLNAPATVCGPGSGGSGSGTNGTKRFDNPVLPFNDGVYFAST | |
| | | | EKSNIIRGWIFGTTLDSKTQSLLIVNNATNVVIKVCEFQFCNDPFLGVYYHKNGSGGSGNITNFCPFDKVFNA | |
| | | | TRFPNVYAWQRTKISDCIADYTVLYNSTSFSTFKCYGVSPSKLIDLCFTSVYADTFLIRFSEVRQIAPGETGV | |
| | | | IADYNYKLPDDFTGCVLAWNTAQQDIGSYFYRSHRAVKLKPFERDLSSDENGVRTLSTYDFNPNVPLDYQAT | |
| | | | RVVVLSFELLNAPATVCGPGSGGSGNKSWMESEFRVYSSANNCTFEYVSQPFLMDLEGKQGNFKNLREF | |
| | | | VFKNIDGYFKIYSKHTPINLVRDLPQGFSALEPLVDLPIGINITRFQTLLALHRSYLTPGDSSSGWTAGAAAY | |
| | | | YVGYLQPRTFLLKYNENGTITDAVDCALDPLSETKCTLKSFTVEKGIYQTSNFRVQPTESIVRFPNITNLCPF | |
| | | | GEVFNATRFASVYAWNRKRISNCVADYSVLYNSASFSTFKCYGVSPTKLNDLCFTNVYADSFVIRGDEVRQIA | |
| | | | PGQTGKIADYNYKLPDDFTGCVIAWNSNNLDSKVGGNYNYLYRLFRKSNLKPFERDISTEIYQAGSTPCNGVE | |
| | | | GFNCYFPLQSYGFQPTNGVGYQPYRVVVLSFELLHAPATVCGPKKSTNLVKNKCVNFNFNGLTGTGVLTES | |
| | | | NKKFLPFQQFGRDIADTTDAVRDPQTLEILDITPCSFGGVSVITPGTNTSNQVAVLYQDVNCTEVPVAIHADQ | |
| | | | LTPTWRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAVEQ | |
| | | | DKNTQEVFAQVKQIYKTPPIKDFGGFNFSQILPDPSKPSKRSFIEDLLFNKVTLADAGFIKQYGDCLGDIAAR | |
| | | | DLICAQKFNGLTVLPPLLTDEMIAQYTSALLAGTITSGWTFGAGAALQIPFAMQMAYRFNGIGVTQNVLYENQ | |
| | | | KLIANQFNSAIGKIQDSLSSTASALGKLQDVVNQNAQALNTLVKQLSSNFGAISSVLNDILSRLDPPEAEVQI | |
| | | | DRLITGRLQSLQTYVTQQLIRAAEIRASANLAATKMSECVLGQSKRVDFCGKGYHLMSFPQSAPHGVVFLHVT | |
| | | | YVPAQEKNFTTAPAICHDGKAHFPREGVFVSNGTHWFVTQRNFYEPQIITTDNTFVSGNCDVVIGIVNNTVYD | |
| | | | PLQSELDSIKEELDKIHKNGSGGSGDIIKLLNEQVNKEMQSSNLYMSMSSWCYTHSLDGAGLFLFDHAAEEYE | |
| | | | HAKKLIIFLNENNVPVQLTSISAPEHKFEGLTQIFQKAYEHEQHISESINNIVDHAIKSKDHATFNFLQWYVA | |
| | | | EQHEEEVLFKDILDKIELIGNENHGLYLADQYVKGIAKSRKSGS | |
| pCoV333 | | His8-3c-RR-SpFN-loop2 | HIHHHGPLEVLFQGPNITNLCPFSQVFNATRFPSVYAWTRERISNCIADYSVLYNSTSFSTFRCYGVSP | 520 |
| | | where MR14 and | TKLNDLCFSNVYADSMVVRGDEVRQIAPSQTGVIADYNYKLPDDFTGCVIAWNSKAKDENGQYFYRLFRKS | |
| | | SARS1(131 like) RBD at N- | KLLPFQRDVSNVTYGSGKNDGCNPSEADCYWPLLKYGFTSSVSQDYQPYRVVVLSFELLNAPATVCGPGS | |
| | | term and MR08 and MR15 are | GGSGNITNLCPFGEVFNATKFPSVYAWERKKISNCVADYSVLYNSTFFSTFKCYGVSATKLNDLCFSNVYAD | |
| | | inserted at NTD loops | SFVVKGDDVRQIAPGQTGVIADYNYKLPDDFMGCVLAWNTRNIDATSTGNYNYKRRYLRHGKLRPFERDIS | |
| | | | NVPFSPDGKPCTPPALNCYWPLNDYGFYTTTGIGYQPYRVVVLSFENKSAPATVCGPGSGGSGSSQCVNL | |
| | | | TTRTQLPPAYTNSFTRGVYYPDKVFRSSVLHSTQDLFLPFFSNVTWFHAIHVGSGGSGNITNFCPFDKVFNA | |
| | | | TRFPNVYAWQRTKISDCIADYTVLYNSTSFSTFKCYGVSPSKLIDLCFTSVYADTFLIRFSEVRQIAPGETGV | |
| | | | IADYNYKLPDDFTGCVLAWNTAQQDIGSYFYRSHRAVKLKPFERDLSSDENGVRTLSTYDFNPNVPLDYQAT | |
| | | | RVVVLSFELLNAPATVCGPGSGGSGSGTNGTKRFDNPVLPFNDGVYFASTEKSNIIRGWIFGTTLDSKTQSL | |
| | | | LIVNNATNVVIKVCEFQFCNDPFLGVYYHKNGSGGSGNMTNICPFDQVFNKTQFPSVYAWERVRISDCVSD | |
| | | | YTVLYNSSASFSTFKCYGVSPTKLNDLCFSGVYADYFVVKGDHVHQIAPGQTGVIADYNYKLPSEFVGCILA | |
| | | | WNTRTIDSKRGFYYRLFRHGNIRPYERDTSNVPYNAAGGTCNQPGTHNCYEPLQDYGFTSTSGVGYQPFR | |
| | | | VVVLSFELLNAPATVCGPGSGGSGNKSWMESEFRVYSSANNCTFEYVSQPFLMDLEGKQGNFKNLREFV | |
| | | | FKNIDGYFKIYSKHTPINLVRDLPQGFSALEPLVDLPIGINITRFQTLLALHRSYLTPGDSSSGWTAGAAAYY | |
| | | | VGYLQPRTFLLKYNENGTITDAVDCALDPLSETKCTLKSFTVEKGIYQTSNFRVQPTESIVRFPNITNLCPFG | |
| | | | EVFNATRFASVYAWNRKRISNCVADYSVLYNSASFSTFKCYGVSPTKLNDLCFTNVYADSFVIRGDEVRQIAP | |
| | | | GQTGKIADYNYKLPDDFTGCVIAWNSNNLDSKVGGNYNYLYRLFRKSNLKPFERDISTEIYQAGSTPCNGVEG | |
| | | | FNCYFPLQSYGFQPTNGVGYQPYRVVVLSFELLHAPATVCGPKKSTNLVKNKCVNFNFNGLTGTGVLTESN | |
| | | | KKFLPFQQFGRDIADTTDAVRDPQTLEILDITPCSFGGVSVITPGTNTSNQVAVLYQDVNCTEVPVAIHADQL | |
| | | | TPTWRVYSTGSNVFQTRAGCLIGAEHVNNSYECDIPIGAGICASYQTQTNSPGSASSVASQSIIAYTMSLGA | |
| | | | ENSVAYSNNSIAIPTNFTISVTTEILPVSMTKTSVDCTMYICGDSTECSNLLLQYGSFCTQLNRALTGIAVEQ | |
| | | | DKNTQEVFAQVKQIYKTPPIKDFGGFNFSQILPDPSKPSKRSFIEDLLFNKVTLADAGFIKQYGDCLGDIAAR | |
| | | | DLICAQKFNGLTVLPPLLTDEMIAQYTSALLAGTITSGWTFGAGAALQIPFAMQMAYRFNGIGVTQNVLYENQ | |
| | | | KLIANQFNSAIGKIQDSLSSTASALGKLQDVVNQNAQALNTLVKQLSSNFGAISSVLNDILSRLDPPEAEVQI | |
| | | | DRLITGRLQSLQTYVTQQLIRAAEIRASANLAATKMSECVLGQSKRVDFCGKGYHLMSFPQSAPHGVVFLHVT | |
| | | | YVPAQEKNFTTAPAICHDGKAHFPREGVFVSNGTHWFVTQRNFYEPQIITTDNTFVSGNCDVVIGIVNNTVYD | |
| | | | PLQSELDSIKEELDKIHKNGSGGSGDIIKLLNEQVNKEMQSSNLYMSMSSWCYTHSLDGAGLFLFDHAAEEYE | |
| | | | HAKKLIIFLNENNVPVQLTSISAPEHKFEGLTQIFQKAYEHEQHISESINNIVDHAIKSKDHATFNFLQWYVA | |
| | | | EQHEEEVLFKDILDKIELIGNENHGLYLADQYVKGIAKSRKSGS | |

TABLE 7

| Strain Design | Amino Acid Sequence |
|---|---|
| empty FN | MDSKGSSQKGSRLLLLLVVSNLLLPQGVVGQESQVRQQFSKDIEKLLNEQVNKEMQSSNLY |
| | MSMSSWCYTHSLDGAGLFLFDHAAEEYEHAKKLIIFLNENNVPVQLTSISAPEHKFEGLTQIFQ |
| | KAYEHEQHISESINNIVDHAIKSKDHATFNFLQWYVAEQHEEEVLFKDILDKIELIGNENHGLYL |
| | ADQYVKGIAKSRKSGS |
| | (SEQ ID NO: 521) |
| WA-1 RFN | MDSKGSSQKGSRLLLLLVVSNLLLPQGVVGQNITNLCPFGEVFNATRFASVYAWNRKRISNC |
| | VADYSVLYNSASFSTFKCYGVSPTKLNDLCFTNVYADSFVIRGDEVRQIAPGQTGKIADYNYK |
| | LPDDFTGCVIAWNSNNLDSKVGGNYNYLRRLFRKSNLKPFERDISTEIYQAGSTPCNGVEGF |
| | NCYFPLQSYGFQPTNGVGYQPYRVVVLSFENKSAPATVCGGGSGGSGESQVRQQFSKDIEK |
| | LLNEQVNKEMQSSNLYMSMSSWCYTHSLDGAGLFLFDHAAEEYEHAKKLIIFLNENNVPVQL |
| | TSISAPEHKFEGLTQIFQKAYEHEQHISESINNIVDHAIKSKDHATFNFLQWYVAEQHEEEVLFK |
| | DILDKIELIGNENHGLYLADQYVKGIAKSRKSGS |
| | (SEQ ID NO: 522) |
| Beta RFN | MDSKGSSQKGSRLLLLLVVSNLLLPQGVVGQNITNLCPFGEVFNATRFASVYAWNRKRISNC |
| | VADYSVLYNSASFSTFKCYGVSPTKLNDLCFTNVYADSFVIRGDEVRQIAPGQTGNIADYNYK |
| | LPDDFTGCVIAWNSNNLDSKVGGNYNYLRRLFRKSNLKPFERDISTEIYQAGSTPCNGVKGF |
| | NCYFPLQSYGFQPTYGVGYQPYRVVVLSFENKSAPATVCGGGSGGSGESQVRQQFSKDIEK |
| | LLNEQVNKEMQSSNLYMSMSSWCYTHSLDGAGLFLFDHAAEEYEHAKKLIIFLNENNVPVQL |
| | TSISAPEHKFEGLTQIFQKAYEHEQHISESINNIVDHAIKSKDHATFNFLQWYVAEQHEEEVLFK |
| | DILDKIELIGNENHGLYLADQYVKGIAKSRKSGS |
| | (SEQ ID NO: 523) |
| BQ1.1 RFN | MDSKGSSQKGSRLLLLLVVSNLLLPQGVVGQNITNLCPFDEVFNATTFASVYAWNRKRISNCV |
| | ADYSVLYNFAPFFAFKCYGVSPTKLNDLCFTNVYADSFVIRGNEVSQIAPGQTGNIADYNYKLP |
| | DDFTGCVIAWNSNKLDSTVGGNYNYRRRLFRKSKLKPFERDISTEIYQAGNKPCNGVAGVNC |
| | YFPLQSYGFRPTYGVGHQPYRVVVLSFENKSAPATVCGGGSGGSGESQVRQQFSKDIEKLL |
| | NEQVNKEMQSSNLYMSMSSWCYTHSLDGAGLFLFDHAAEEYEHAKKLIIFLNENNVPVQLTS |
| | SAPEHKFEGLTQIFQKAYEHEQHISESINNIVDHAIKSKDHATFNFLQWYVAEQHEEEVLFKDIL |
| | DKIELIGNENHGLYLADQYVKGIAKSRKSGS |
| | (SEQ ID NO: 524) |
| SARS-CoV-1 | MDSKGSSQKGSRLLLLLVVSNLLLPQGVVGQNITNLCPFGEVFNATKFPSVYAWERKKISNCV |
| RFN | ADYSVLYNSTFFSTFKCYGVSATKLNDLCFSNVYADSFVVKGDDVRQIAPGQTGVIADYNYKL |
| | PDDFMGCVLAWNTRNIDATSTGNYNYKRRYLRHGKLRPFERDISNVPFSPDGKPCTPPALNC |
| | YWPLNDYGFYTTTGIGYQPYRVVVLSFENKSAPATVCGGGSGGSGESQVRQQFSKDIEKLL |
| | NEQVNKEMQSSNLYMSMSSWCYTHSLDGAGLFLFDHAAEEYEHAKKLIIFLNENNVPVQLTSI |
| | SAPEHKFEGLTQIFQKAYEHEQHISESINNIVDHAIKSKDHATFNFLQWYVAEQHEEEVLFKDIL |
| | DKIELIGNENHGLYLADQYVKGIAKSRKSGS |
| | (SEQ ID NO: 525) |
| Khosta-2 RFN | MDSKGSSQKGSRLLLLLVVSNLLLPQGVVGQNMTNICPFDQVFNKTQFPSVYAWERVRISDC |
| | VSDYTVLYNSSASFSTFKCYGVSPTKLNDLCFSGVYADYFVVKGDHVHQIAPGQTGVIADYNY |
| | KLPSEFVGCILAWNTRTIDSKRGFYYRLFRHGNIRPYERDTSNVPYNAAGGTCNQPGTHNCY |
| | EPLQDYGFTSTSGVGYQPFRVVVLSFENKSAPATVCGGGSGGSGESQVRQQFSKDIEKLLN |
| | EQVNKEMQSSNLYMSMSSWCYTHSLDGAGLFLFDHAAEEYEHAKKLIIFLNENNVPVQLTSIS |
| | APEHKFEGLTQIFQKAYEHEQHISESINNIVDHAIKSKDHATFNFLQWYVAEQHEEEVLFKDILD |
| | KIELIGNENHGLYLADQYVKGIAKSRKSGS |
| | (SEQ ID NO: 526) |
| BANAL20-247 | MDSKGSSQKGSRLLLLLVVSNLLLPQGVVGQNITNFCPFDKVFNATRFPNVYAWQRTKISDCI |
| RFN | ADYTVLYNSTSFSTFKCYGVSPSKLIDLCFTSVYADTFLIRFSEVRQIAPGETGVIADYNYKLPD |
| | DFTGCVLAWNTAQQDIGSYFYRSHRAVKLKPFERDLSSDENGVRTLSTYDFNPNVPLDYQAT |
| | RVVVLSFENKSAPATVCGGGSGGSGESQVRQQFSKDIEKLLNEQVNKEMQSSNLYMSMSS |
| | WCYTHSLDGAGLFLFDHAAEEYEHAKKLIIFLNENNVPVQLTSISAPEHKFEGLTQIFQKAYEH |
| | EQHISESINNIVDHAIKSKDHATFNFLQWYVAEQHEEEVLFKDILDKIELIGNENHGLYLADQYV |
| | KGIAKSRKSGS |
| | (SEQ ID NO: 527) |
| MERS RFN | MDSKGSSQKGSRLLLLLVVSNLLLPQGVVGQEGVECDFSPLLSGTPPQVYNFKRLVFTNCNY |
| | NLTKLLSLFSVNDFTCSQISPAAIASNCYSSLILDYFSYPLSMKSDLSVSSAGPISQFNYKQSFS |
| | NPTCLILATVPHNLTTITKPLKYSYINKCSRLLSDDRTEVPQLVNANQYSPCVSIVPSTVAEDGD |
| | YYRKQLSPLEGGGWLVASGSTVAMTEQLQMGFGITVQNGTDTNSVCGGGSGGSGESQVR |
| | QQFSKDIEKLLNEQVNKEMQSSNLYMSMSSWCYTHSLDGAGLFLFDHAAEEYEHAKKLIIFLN |
| | ENNVPVQLTSISAPEHKFEGLTQIFQKAYEHEQHISESINNIVDHAIKSKDHATFNFLQWYVAE |
| | QHEEEVLFKDILDKIELIGNENHGLYLADQYVKGIAKSRKSGS |
| | (SEQ ID NO: 528) |
| WA-1 SpFN | MDSKGSSQKGSRLLLLLVVSNLLLPQGVVGQSSQCVNLTTRTQLPPAYTNSFTRGVYYPDKV |
| | FRSSVLHSTQDLFLPFFSNVTWFHAIHVSGTNGTKRFDNPVLPFNDGVYFASTEKSNIIRGWIF |
| | GTTLDSKTQSLLIVNNATNVVIKVCEFQFCNDPFLGVYYHKNNKSWMESEFRVYSSANNCTF |
| | EYVSQPFLMDLEGKQGNFKNLREFVFKNIDGYFKIYSKHTPINLVRDLPQGFSALEPLVDLPIGI |
| | NITRFQTLLALHRSYLTPGDSSSGWTAGAAAYYVGYLQPRTFLLKYNENGTITDAVDCALDPL |
| | SETKCTLKSFTVEKGIYQTSNFRVQPTESIVRFPNITNLCPFGEVFNATRFASVYAWNRKRISN |
| | CVADYSVLYNSASFSTFKCYGVSPTKLNDLCFTNVYADSFVIRGDEVRQIAPGQTGKIADYNY |
| | KLPDDFTGCVIAWNSNNLDSKVGGNYNYLYRLFRKSNLKPFERDISTEIYQAGSTPCNGVEGF |
| | NCYFPLQSYGFQPTNGVGYQPYRVVVLSFELLHAPATVCGPKKSTNLVKNKCVNFNFNGLTG |
| | TGVLTESNKKFLPFQQFGRDIADTTDAVRDPQTLEILDITPCSFGGVSVITPGTNTSNQVAVLY |
| | QDVNCTEVPVAIHADQLTPTWRVYSTGSNVFQTRAGCLIGAEHVNNSYECDIPIGAGICASYQ |
| | TQTNSPGSASSVASQSIIAYTMSLGAENSVAYSNNSIAIPTNFTISVTTEILPVSMTKTSVDCTM |
| | YICGDSTECSNLLLQYGSFCTQLNRALTGIAVEQDKNTQEVFAQVKQIYKTPPIKDFGGFNFSQ |
| | ILPDPSKPSKRSFIEDLLFNKVTLADAGFIKQYGDCLGDIAARDLICAQKFNGLTVLPPLLTDEMI |
| | AQYTSALLAGTITSGWTFGAGAALQIPFAMQMAYRFNGIGVTQNVLYENQKLIANQFNSAIGKI |
| | QDSLSSTASALGKLQDVVNQNAQALNTLVKQLSSNFGAISSVLNDILSRLDPPEAEVQIDRLIT |
| | GRLQSLQTYVTQQLIRAAEIRASANLAATKMSECVLGQSKRVDFCGKGYHLMSFPQSAPHGV |
| | VFLHVTYVPAQEKNFTTAPAICHDGKAHFPREGVFVSNGTHWFVTQRNFYEPQIITTDNTFVS |
| | GNCDVVIGIVNNTVYDPLQSELDSIKEELDKIHKNGSGGSGDIIKLLNEQVNKEMQSSNLYMSM |
| | SSWCYTHSLDGAGLFLFDHAAEEYEHAKKLIIFLNENNVPVQLTSISAPEHKFEGLTQIFQKAY |
| | EHEQHISESINNIVDHAIKSKDHATFNFLQWYVAEQHEEEVLFKDILDKIELIGNENHGLYLADQ |
| | YVKGIAKSRKSGS |
| | (SEQ ID NO: 529) |
| Beta SpFN | MDSKGSSQKGSRLLLLLVVSNLLLPQGVVGQSSQCVNFTTRTQLPPAYTNSFTRGVYYPDKV |
| | FRSSVLHSTQDLFLPFFSNVTWFHAIHVSGTNGTKRFANPVLPFNDGVYFASTEKSNIIRGWIF |
| | GTTLDSKTQSLLIVNNATNVVIKVCEFQFCNDPFLGVYYHKNNKSWMESEFRVYSSANNCTF |
| | EYVSQPFLMDLEGKQGNFKNLREFVFKNIDGYFKIYSKHTPINLVRGLPQGFSALEPLVDLPIGI |
| | NITRFQTLHISYLTPGDSSSGWTAGAAAYYVGYLQPRTFLLKYNENGTITDAVDCALDPLSETK |
| | CTLKSFTVEKGIYQTSNFRVQPTESIVRFPNITNLCPFGEVFNATRFASVYAWNRKRISNCVAD |
| | YSVLYNSASFSTFKCYGVSPTKLNDLCFTNVYADSFVIRGDEVRQIAPGQTGNIADYNYKLPD |
| | DFTGCVIAWNSNNLDSKVGGNYNYLYRLFRKSNLKPFERDISTEIYQAGSTPCNGVKGFNCY |
| | FPLQSYGFQPTYGVGYQPYRVVVLSFELLHAPATVCGPKKSTNLVKNKCVNFNFNGLTGTGV |
| | LTESNKKFLPFQQFGRDIADTTDAVRDPQTLEILDITPCSFGGVSVITPGTNTSNQVAVLYQGV |
| | NCTEVPVAIHADQLTPTWRVYSTGSNVFQTRAGCLIGAEHVNNSYECDIPIGAGICASYQTQT |
| | NSPGSASSVASQSIIAYTMSLGVENSVAYSNNSIAIPTNFTISVTTEILPVSMTKTSVDCTMYICG |
| | DSTECSNLLLQYGSFCTQLNRALTGIAVEQDKNTQEVFAQVKQIYKTPPIKDFGGFNFSQILPD |
| | PSKPSKRSFIEDLLFNKVTLADAGFIKQYGDCLGDIAARDLICAQKFNGLTVLPPLLTDEMIAQY |
| | TSALLAGTITSGWTFGAGAALQIPFAMQMAYRFNGIGVTQNVLYENQKLIANQFNSAIGKIQDS |
| | LSSTASALGKLQDVVNQNAQALNTLVKQLSSNFGAISSVLNDILSRLDPPEAEVQIDRLITGRLQ |
| | SLQTYVTQQLIRAAEIRASANLAATKMSECVLGQSKRVDFCGKGYHLMSFPQSAPHGVVFLH |
| | VTYVPAQEKNFTTAPAICHDGKAHFPREGVFVSNGTHWFVTQRNFYEPQIITTDNTFVSGNC |
| | DVVIGIVNNTVYDPLQSELDSIKEELDKIHKNGSGGSGDIIKLLNEQVNKEMQSSNLYMSMSS |
| | WCYTHSLDGAGLFLFDHAAEEYEHAKKLIIFLNENNVPVQLTSISAPEHKFEGLTQIFQKAYEH |
| | EQHISESINNIVDHAIKSKDHATFNFLQWYVAEQHEEEVLFKDILDKIELIGNENHGLYLADQYV |
| | KGIAKSRKSGS |
| | (SEQ ID NO: 530) |
| BQ1.1 SpFN | MDSKGSSQKGSRLLLLLVVSNLLLPQGVVGQSSQCVNLITRTQSYTNSFTRGVYYPDKVFRS |
| | SVLHSTQDLFLPFFSNVTWFHAISGTNGTKRFDNPVLPFNDGVYFASTEKSNIIRGWIFGTTLD |
| | SKTQSLLIVNNATNVVIKVCEFQFCNDPFLDVYYHKNNKSWMESEFRVYSSANNCTFEYVSQ |
| | PFLMDLEGKQGNFKNLREFVFKNIDGYFKIYSKHTPINLGRDLPQGFSALEPLVDLPIGINITRF |
| | QTLLALHRSYLTPGDSSSGWTAGAAAYYVGYLQPRTFLLKYNENGTITDAVDCALDPLSETKQ |
| | TLKSFTVEKGIYQTSNFRVQPTESIVRFPNITNLCPFDEVFNATTFASVYAWNRKRISNCVADY |
| | SVLYNFAPFFAFKCYGVSPTKLNDLCFTNVYADSFVIRGNEVSQIAPGQTGNIADYNYKLPDDR |
| | TGCVIAWNSNKLDSTVGGNYNYRYRLFRKSKLKPFERDISTEIYQAGNKPCNGVAGVNCYFPL |
| | QSYGFRPTYGVGHQPYRVVVLSFELLHAPATVCGPKKSTNLVKNKCVNFNFNGLTGTGVLTE |
| | SNKKFLPFQQFGRDIADTTDAVRDPQTLEILDITPCSFGGVSVITPGTNTSNQVAVLYQGVNCT |
| | EVPVAIHADQLTPTWRVYSTGSNVFQTRAGCLIGAEYVNNSYECDIPIGAGICASYQTQTKSH |
| | GSASSVASQSIIAYTMSLGAENSVAYSNNSIAIPTNFTISVTTEILPVSMTKTSVDCTMYICGDST |
| | ECSNLLLQYGSFCTQLKRALTGIAVEQDKNTQEVFAQVKQIYKTPPIKYFGGFNFSQILPDPSK |
| | PSKRSFIEDLLFNKVTLADAGFIKQYGDCLGDIAARDLICAQKFNGLTVLPPLLTDEMIAQYTSA |
| | LLAGTITSGWTFGAGAALQIPFAMQMAYRFNGIGVTQNVLYENQKLIANQFNSAIGKIQDSLSS |
| | TASALGKLQDVVNHNAQALNTLVKQLSSKFGAISSVLNDILSRLDPPEAEVQIDRLITGRLQSLQ |
| | TYVTQQLIRAAEIRASANLAATKMSECVLGQSKRVDFCGKGYHLMSFPQSAPHGVVFLHVTY |
| | VPAQEKNFTTAPAICHDGKAHFPREGVFVSNGTHWFVTQRNFYEPQIITTDNTFVSGNCDVVI |
| | GIVNNTVYDPLQSELDSIKEELDKIHKNGSGGSGDIIKLLNEQVNKEMQSSNLYMSMSSWCYT |
| | HSLDGAGLFLFDHAAEEYEHAKKLIIFLNENNVPVQLTSISAPEHKFEGLTQIFQKAYEHEQHIS |
| | ESINNIVDHAIKSKDHATFNFLQWYVAEQHEEEVLFKDILDKIELIGNENHGLYLADQYVKGIAK |
| | SRKSGS |
| | (SEQ ID NO: 531) |
| SARS-CoV-1 | MDSKGSSQKGSRLLLLLVVSNLLLPQGVVGQSDLDRCTTFDDVQAPNYTQHTSSMRGVYYP |
| SpFN | DEIFRSDTLYLTQDLFLPFYSNVTGFHTINHTFGNPVIPFKDGIYFAATEKSNVVRGWVFGSTM |
| | NNKSQSVIIINNSTNVVIRACNFELCDNPFFAVSKPMGTQTHTMIFDNAFNCTFEYISDAFSLDV |
| | SEKSGNFKHLREFVFKNKDGFLYVYKGYQPIDVVRDLPSGFNTLKPIFKLPLGINITNFRAILTAF |
| | SPAQDIWGTSAAAYFVGYLKPTTFMLKYDENGTITDAVDCSQNPLAELKCSVKSFEIDKGIYQT |
| | SNFRVVPSGDVVRFPNITNLCPFGEVFNATKFPSVYAWERKKISNCVADYSVLYNSTFFSTFK |
| | CYGVSATKLNDLCFSNVYADSFVVKGDDVRQIAPGQTGVIADYNYKLPDDFMGCVLAWNTR |
| | NIDATSTGNYNYKYRYLRHGKLRPFERDISNVPFSPDGKPCTPPALNCYWPLNDYGFYTTTGI |
| | GYQPYRVVVLSFELLNAPATVCGPKLSTDLIKNQCVNFNFNGLTGTGVLTPSSKRFQPFQQF |
| | GRDVSDFTDSVRDPKTSEILDISPCAFGGVSVITPGTNASSEVAVLYQDVNCTDVSTAIHADQL |
| | TPAWRIYSTGNNVFQTQAGCLIGAEHVDTSYECDIPIGAGICASYHTVSLLRSTSQKSIVAYTM |
| | SLGADSSIAYSNNTIAIPTNFSISITTEVMPVSMAKTSVDCNMYICGDSTECANLLLQYGSFCTQ |
| | LNRALSGIAAEQDRNTREVFAQVKQMYKTPTLKYFGGFNFSQILPDPLKPTKRSFIEDLLFNKV |
| | TLADAGFMKQYGECLGDINARDLICAQKFNGLTVLPPLLTDDMIAAYTAALVSGTATAGWTFG |
| | AGAALQIPFAMQMAYRFNGIGVTQNVLYENQKQIANQFNKAISQIQESLTTTSTALGKLQDVVN |
| | QNAQALNTLVKQLSSNFGAISSVLNDILSRLDPPEAEVQIDRLITGRLQSLQTYVTQQLIRAAEI |
| | RASANLAATKMSECVLGQSKRVDFCGKGYHLMSFPQAAPHGVVFLHVTYVPSQERNFTTAP |
| | AICHEGKAYFPREGVFVFNGTSWFITQRNFFSPQIITTDNTFVSGNCDVVIGIINNTVYDPLQSE |
| | LDSIKEELDKIHKNGSGGSGDIIKLLNEQVNKEMQSSNLYMSMSSWCYTHSLDGAGLFLFDHA |
| | AEEYEHAKKLIIFLNENNVPVQLTSISAPEHKFEGLTQIFQKAYEHEQHISESINNIVDHAIKSKD |
| | HATFNFLQWYVAEQHEEEVLFKDILDKIELIGNENHGLYLADQYVKGIAKSRKSGS |
| | (SEQ ID NO: 532) |
| Khosta-2 | MDSKGSSQKGSRLLLLLVVSNLLLPQGVVGQSSQDGCATLSTKLTPTQLQVNSTRRGFYYFD |
| SpFN | DIYRSSLRVLVPGYFLPFGTNITRYQAQVINWTAIFFDNPVIPFNDGVYFAALEHSNVVRGWIF |
| | GTTLDNTTQSAIVVNNATHILISVCYFNVCKEPMFAVSNFKPYKSWVYDRANNCTFNRAYPLNI |
| | STTPEPGKFKALREQVFRYQDGFLYIYHSYESINSDTLPPGFSVLKPMLKLPLGLNITRFRVVM |
| | AMHSLTTSNFNTHSVNYFVGHLKPLTMLVEFSPNGTIIDAIDCSQDPLSELKCTTKSFSVEKGIY |
| | QTSNFRVSPSLDVVRFPNMTNICPFDQVFNKTQFPSVYAWERVRISDCVSDYTVLYNSSASF |
| | STFKCYGVSPTKLNDLCFSGVYADYFVVKGDHVHQIAPGQTGVIADYNYKLPSEFVGCILAWN |
| | TRTIDSKRGFYYRLFRHGNIRPYERDTSNVPYNAAGGTCNQPGTHNCYEPLQDYGFTSTSGV |
| | GYQPFRVVVLSFELLNAPATVCGPKQSTDLVKNKCVNFNFNGLTGTGVLTDSNKKFQPFQQF |
| | GRDSADFTDSVKDPKTLEILDITPCSYGGVSVITPGTNTSDSVAVLYQDVNCTDVPTMLHMDQ |
| | VSNDWRVYAVNTDGNMFQTQAGCLVGATYDNTSYECDIPVGAGVCAKFQTTGSASQSSILA |
| | YTMSLGEDSNVAYSNNSIAIPTNFTISVTTEVLPVSMTKTAVDCNMYICGDSTECSSLLLQYGS |
| | FCQQLNRALAGVSVEQDKNTQDVFAQVKSIYKVSAIKDFGGFNFSQILPDPAKPSKRSFIEDLL |
| | YNKVTLADPGFMKQYGDCLGGVNARDLICAQKFNGLTVLPPLLTDDMIAAYTSALVSGTALSG |
| | FTFGAGAALQIPFAMQMAYRFNGIGVTQNVLYENQKQIANQFNKAISQIQESLTTTATALGKLQ |
| | DVINQNAIALNTLVKQLSSNFGAISSVLNDILSRLDPPEAEVQIDRLITGRLQSLQTYVTQQLIRA |
| | ADIRASANLAATKMSECVLGQSKRVDFCGKGYHLMSFPQAAPHGVVFLHVTYVPSQDKNFTT |
| | APAICHKGKAYFPREGVFVTNGTHWFVTQRNFYQPEVITTENTFESGNCDVVIGIVNNTVYDP |
| | LQSELDSIKEELDKIHKNGSGGSGDIIKLLNEQVNKEMQSSNLYMSMSSWCYTHSLDGAGLFL |
| | FDHAAEEYEHAKKLIIFLNENNVPVQLTSISAPEHKFEGLTQIFQKAYEHEQHISESINNIVDHAIK |
| | SKDHATFNFLQWYVAEQHEEEVLFKDILDKIELIGNENHGLYLADQYVKGIAKSRKSGS |
| | (SEQ ID NO: 533) |
| MERS SpFN | MDSKGSSQKGSRLLLLLVVSNLLLPQGVVGQYVDVGPDSVKSACIEVDIQQTFFDKTWPRPID |
| | VSKADGIIYPQGRTYSNITITYQGLFPYQGDHGDMYVYSAGHATGTTPQKLFVANYSQDVKQF |
| | ANGFVVRIGAAANSTGTVIISPSTSATIRKIYPAFMLGSSVGNFSDGKMGRFFNHTLVLLPDGC |
| | GTLLRAFYCILEPRSGNHCPAGNSYTSFATYHTPATDCSDGNYNRNASLNSFKEYFNLRNCT |
| | FMYTYNITEDEILEWFGITQTAQGVHLFSSRYVDLYGGNMFQFATLPVYDTIKYYSIIPHSIRSIQ |
| | SDRKAWAAFYVYKLQPLTFLLDFSVDGYIRRAIDCGFNDLSQLHCSYESFDVESGVYSVSSFE |
| | AKPSGSVVEQAEGVECDFSPLLSGTPPQVYNFKRLVFTNCNYNLTKLLSLFSVNDFTCSQISP |
| | AAIASNCYSSLILDYFSYPLSMKSDLSVSSAGPISQFNYKQSFSNPTCLILATVPHNLTTITKPLK |
| | YSYINKCSRFLSDDRTEVPQLVNANQYSPCVSIVPSTVWEDGDYYRKQLSPLEGGGWLVAS |
| | GSTVAMTEQLQMGFGITVQYGTDTNSVCPKLEFANDTKIASQLGNCVEYSLYGVSGRGVFQN |
| | CTAVGVRQQRFVYDAYQNLVGYYSDDGNYYCLRACVSVPVSVIYDKETKTHATLFGSVACEH |
| | ISSTMSQYSRSTRSMLKRRDSTYGPLQTPVGCVLGLVNSSLFVEDCKLPLGQSLCALPDTPST |
| | LTPASVGSVPGEMRLASIAFNHPIQVDQLNSSYFKLSIPTNFSFGVTQEYIQTTIQKVTVDCKQY |
| | VCNGFQKCEQLLREYGQFCSKINQALHGANLRQDDSVRNLFASVKSSQSSPIIPGFGGDFNL |
| | TLLEPVSISTGSRSARSAIEDLLFDKVTIADPGYMQGYDDCMQQGPASARDLICAQYVAGYKV |
| | LPPLMDVNMEAAYTSSLLGSIAGVGWTAGLSSFAAIPFAQSIFYRLNGVGITQQVLSENQKLIA |
| | NKFNQALGAMQTGFTTTNEAFHKVQDAVNNNAQALSKLASELSNTFGAISASIGDIIQRLDPPE |
| | QDAQIDRLINGRLTTLNAFVAQQLVRSESAALSAQLAKDKVNECVKAQSKRSGFCGQGTHIVS |
| | FVVNAPNGLYFMHVGYYPSNHIEVVSAYGLCDAANPTNCIAPVNGYFIKTNNTRIVDEWSYTG |
| | SSFYAPEPITSLNTKYVAPQVTYQNISTNLPPPLLGNSTGIDFQDELDEIHKQGSGGSGDIIKLL |
| | NEQVNKEMQSSNLYMSMSSWCYTHSLDGAGLFLFDHAAEEYEHAKKLIIFLNENNVPVQLTS |
| | SAPEHKFEGLTQIFQKAYEHEQHISESINNIVDHAIKSKDHATFNFLQWYVAEQHEEEVLFKDIL |
| | DKIELIGNENHGLYLADQYVKGIAKSRKSGS |
| | (SEQ ID NO: 534) |
| BQ1.1_S2P | MDSKGSSQKGSRLLLLLVVSNLLLPQGVVGQSSQCVNLITRTQSYTNSFTRGVYYPDKVFRS |
| | SVLHSTQDLFLPFFSNVTWFHAISGTNGTKRFDNPVLPFNDGVYFASTEKSNIIRGWIFGTTLD |
| | SKTQSLLIVNNATNVVIKVCEFQFCNDPFLDVYYHKNNKSWMESEFRVYSSANNCTFEYVSQ |
| | PFLMDLEGKQGNFKNLREFVFKNIDGYFKIYSKHTPINLGRDLPQGFSALEPLVDLPIGINITRF |
| | QTLLALHRSYLTPGDSSSGWTAGAAAYYVGYLQPRTFLLKYNENGTITDAVDCALDPLSETKC |
| | TLKSFTVEKGIYQTSNFRVQPTESIVRFPNITNLCPFDEVFNATTFASVYAWNRKRISNCVADY |
| | SVLYNFAPFFAFKCYGVSPTKLNDLCFTNVYADSFVIRGNEVSQIAPGQTGNIADYNYKLPDDF |
| | TGCVIAWNSNKLDSTVGGNYNYRYRLFRKSKLKPFERDISTEIYQAGNKPCNGVAGVNCYFPL |
| | QSYGFRPTYGVGHQPYRVVVLSFELLHAPATVCGPKKSTNLVKNKCVNFNFNGLTGTGVLTE |
| | SNKKFLPFQQFGRDIADTTDAVRDPQTLEILDITPCSFGGVSVITPGTNTSNQVAVLYQGVNCT |
| | EVPVAIHADQLTPTWRVYSTGSNVFQTRAGCLIGAEYVNNSYECDIPIGAGICASYQTQTKSH |
| | GSASSVASQSIIAYTMSLGAENSVAYSNNSIAIPTNFTISVTTEILPVSMTKTSVDCTMYICGDST |
| | ECSNLLLQYGSFCTQLKRALTGIAVEQDKNTQEVFAQVKQIYKTPPIKYFGGFNFSQILPDPSK |
| | PSKRSFIEDLLFNKVTLADAGFIKQYGDCLGDIAARDLICAQKFNGLTVLPPLLTDEMIAQYTSA |
| | LLAGTITSGWTFGAGAALQIPFAMQMAYRFNGIGVTQNVLYENQKLIANQFNSAIGKIQDSLSS |
| | TASALGKLQDVVNHNAQALNTLVKQLSSKFGAISSVLNDILSRLDPPEAEVQIDRLITGRLQSLQ |
| | TYVTQQLIRAAEIRASANLAATKMSECVLGQSKRVDFCGKGYHLMSFPQSAPHGVVFLHVTY |
| | VPAQEKNFTTAPAICHDGKAHFPREGVFVSNGTHWFVTQRNFYEPQIITTDNTFVSGNCDVVI |
| | GIVNNTVYDPLQPELDSFKEELDKYFKNHTSPDVDLGDISGINASVVNIQKEIDRLNEVAKNLNE |
| | SLIDLQELGKYEQYIKWPWYIWLGFIAGLIAIVMVTIMLCCMTSCCSCLKGCCSCGSCCKFDE |
| | DDSEPVLKGVKLHY |
| | (SEQ ID NO: 538) |
| SARS_S2P | MDSKGSSQKGSRLLLLLVVSNLLLPQGVVGQSDLDRCTTFDDVQAPNYTQHTSSMRGVYYP |
| | DEIFRSDTLYLTQDLFLPFYSNVTGFHTINHTFGNPVIPFKDGIYFAATEKSNVVRGWVFGSTM |
| | NNKSQSVIIINNSTNVVIRACNFELCDNPFFAVSKPMGTQTHTMIFDNAFNCTFEYISDAFSLDV |
| | SEKSGNFKHLREFVFKNKDGFLYVYKGYQPIDVVRDLPSGFNTLKPIFKLPLGINITNFRAILTAF |
| | SPAQDIWGTSAAAYFVGYLKPTTFMLKYDENGTITDAVDCSQNPLAELKCSVKSFEIDKGIYQT |
| | SNFRVVPSGDVVRFPNITNLCPFGEVFNATKFPSVYAWERKKISNCVADYSVLYNSTFFSTFK |
| | CYGVSATKLNDLCFSNVYADSFVVKGDDVRQIAPGQTGVIADYNYKLPDDFMGCVLAWNTR |
| | NIDATSTGNYNYKYRYLRHGKLRPFERDISNVPFSPDGKPCTPPALNCYWPLNDYGFYTTTG |
| | GYQPYRVVVLSFELLNAPATVCGPKLSTDLIKNQCVNFNFNGLTGTGVLTPSSKRFQPFQQF |
| | GRDVSDFTDSVRDPKTSEILDISPCAFGGVSVITPGTNASSEVAVLYQDVNCTDVSTAIHADQL |
| | TPAWRIYSTGNNVFQTQAGCLIGAEHVDTSYECDIPIGAGICASYHTVSLLRSTSQKSIVAYTM |
| | SLGADSSIAYSNNTIAIPTNFSISITTEVMPVSMAKTSVDCNMYICGDSTECANLLLQYGSFCTQ |
| | LNRALSGIAAEQDRNTREVFAQVKQMYKTPTLKYFGGFNFSQILPDPLKPTKRSFIEDLLFNKV |
| | TLADAGFMKQYGECLGDINARDLICAQKFNGLTVLPPLLTDDMIAAYTAALVSGTATAGWTFG |
| | AGAALQIPFAMQMAYRFNGIGVTQNVLYENQKQIANQFNKAISQIQESLTTTSTALGKLQDVVN |
| | QNAQALNTLVKQLSSNFGAISSVLNDILSRLDPPEAEVQIDRLITGRLQSLQTYVTQQLIRAAEI |
| | RASANLAATKMSECVLGQSKRVDFCGKGYHLMSFPQAAPHGVVFLHVTYVPSQERNFTTAP |
| | AICHEGKAYFPREGVFVFNGTSWFITQRNFFSPQIITTDNTFVSGNCDVVIGIINNTVYDPLQPE |
| | LDSFKEELDKYFKNHTSPDVDLGDISGINASVVNIQKEIDRLNEVAKNLNESLIDLQELGKYEQY |
| | IKWPWYVWLGFIAGLIAIVMVTILLCCMTSCCSCLKGACSCGSCCKFDEDDSEPVLKGVKLHY |
| | (SEQ ID NO: 539) |
| KHOSTA2_S2P | MDSKGSSQKGSRLLLLLVVSNLLLPQGVVGQQDGCATLSTKLTPTQLQVNSTRRGFYYFDDI |
| | YRSSLRVLVPGYFLPFGTNITRYQAQVINWTAIFFDNPVIPFNDGVYFAALEHSNVVRGWIFGT |
| | TLDNTTQSAIVVNNATHILISVCYFNVCKEPMFAVSNFKPYKSWVYDRANNCTFNRAYPLNIST |
| | TPEPGKFKALREQVFRYQDGFLYIYHSYESINSDTLPPGFSVLKPMLKLPLGLNITRFRVVMAM |
| | HSLTTSNFNTHSVNYFVGHLKPLTMLVEFSPNGTIIDAIDCSQDPLSELKCTTKSFSVEKGIYQT |
| | SNFRVSPSLDVVRFPNMTNICPFDQVFNKTQFPSVYAWERVRISDCVSDYTVLYNSSASFST |
| | FKCYGVSPTKLNDLCFSGVYADYFVVKGDHVHQIAPGQTGVIADYNYKLPSEFVGCILAWNT |
| | RTIDSKRGFYYRLFRHGNIRPYERDTSNVPYNAAGGTCNQPGTHNCYEPLQDYGFTSTSGV |
| | GYQPFRVVVLSFELLNAPATVCGPKQSTDLVKNKCVNFNFNGLTGTGVLTDSNKKFQPFQQF |
| | GRDSADFTDSVKDPKTLEILDITPCSYGGVSVITPGTNTSDSVAVLYQDVNCTDVPTMLHMDQ |
| | VSNDWRVYAVNTDGNMFQTQAGCLVGATYDNTSYECDIPVGAGVCAKFQTTTRAKQSSILA |
| | YTMSLGEDSNVAYSNNSIAIPTNFTISVTTEVLPVSMTKTAVDCNMYICGDSTECSSLLLQYGS |
| | FCQQLNRALAGVSVEQDKNTQDVFAQVKSIYKVSAIKDFGGFNFSQILPDPAKPSKRSFIEDLL |
| | YNKVTLADPGFMKQYGDCLGGVNARDLICAQKFNGLTVLPPLLTDDMIAAYTSALVSGTALSG |
| | FTFGAGAALQIPFAMQMAYRFNGIGVTQNVLYENQKQIANQFNKAISQIQESLTTTATALGKLQ |
| | DVINQNAIALNTLVKQLSSNFGAISSVLNDILSRLDPPEAEVQIDRLITGRLQSLQTYVTQQLIRA |
| | ADIRASANLAATKMSECVLGQSKRVDFCGKGYHLMSFPQAAPHGVVFLHVTYVPSQDKNFT |
| | APAICHKGKAYFPREGVFVTNGTHWFVTQRNFYQPEVITTENTFESGNCDVVIGIVNNTVYDP |
| | LQPELESFKEELDKYFKNHTSPDVDFDDISGINASVVDIKKEIEHLNEIAKSLNESLIDLQELGKY |
| | EQYIKWPWYVWLGFIAGLIAIIMATIMLCCMTSCCSCLKGLCSCGSCCKFDDDHSEPVLKGVK |
| | LHY |
| | (SEQ ID NO: 540) |
| BQ1.1_S2P | MDSKGSSQKGSRLLLLLVVSNLLLPQGVVGQSSQCVNLITRTQSYTNSFTRGVYYPDKVFRS |
| | SVLHSTQDLFLPFFSNVTWFHAISGTNGTKRFDNPVLPFNDGVYFASTEKSNIIRGWIFGTTLD |
| | SKTQSLLIVNNATNVVIKVCEFQFCNDPFLDVYYHKNNKSWMESEFRVYSSANNCTFEYVSQ |
| | PFLMDLEGKQGNFKNLREFVFKNIDGYFKIYSKHTPINLGRDLPQGFSALEPLVDLPIGINITRF |
| | QTLLALHRSYLTPGDSSSGWTAGAAAYYVGYLQPRTFLLKYNENGTITDAVDCALDPLSETKC |
| | TLKSFTVEKGIYQTSNFRVQPTESIVRFPNITNLCPFDEVFNATTFASVYAWNRKRISNCVADY |
| | SVLYNFAPFFAFKCYGVSPTKLNDLCFTNVYADSFVIRGNEVSQIAPGQTGNIADYNYKLPDDF |
| | TGCVIAWNSNKLDSTVGGNYNYRYRLFRKSKLKPFERDISTEIYQAGNKPCNGVAGVNCYFPL |
| | QSYGFRPTYGVGHQPYRVVVLSFELLHAPATVCGPKKSTNLVKNKCVNFNFNGLTGTGVLTE |
| | SNKKFLPFQQFGRDIADTTDAVRDPQTLEILDITPCSFGGVSVITPGTNTSNQVAVLYQGVNCT |
| | EVPVAIHADQLTPTWRVYSTGSNVFQTRAGCLIGAEYVNNSYECDIPIGAGICASYQTQTKSH |
| | GSASSVASQSIIAYTMSLGAENSVAYSNNSIAIPTNFTISVTTEILPVSMTKTSVDCTMYICGDST |
| | ECSNLLLQYGSFCTQLKRALTGIAVEQDKNTQEVFAQVKQIYKTPPIKYFGGFNFSQILPDPSK |
| | PSKRSFIEDLLFNKVTLADAGFIKQYGDCLGDIAARDLICAQKFNGLTVLPPLLTDEMIAQYTSA |
| | LLAGTITSGWTFGAGAALQIPFAMQMAYRFNGIGVTQNVLYENQKLIANQFNSAIGKIQDSLSS |
| | TASALGKLQDVVNHNAQALNTLVKQLSSKFGAISSVLNDILSRLDPPEAEVQIDRLITGRLQSLQ |
| | TYVTQQLIRAAEIRASANLAATKMSECVLGQSKRVDFCGKGYHLMSFPQSAPHGVVFLHVTY |
| | VPAQEKNFTTAPAICHDGKAHFPREGVFVSNGTHWFVTQRNFYEPQIITTDNTFVSGNCDVVI |
| | GIVNNTVYDPLQPELDSFKEELDKYFKNHTSPDVDLGDISGINASVVNIQKEIDRLNEVAKNLNE |
| | SLIDLQELGKYEQYIKWPWYIWLGFIAGLIAIVMVTIMLCCMTSCCSCLKGCCSCGSCCKFDE |
| | DDSEPVLKGVKLHY |
| | (SEQ ID NO: 538) |
| SARS_S2P | MDSKGSSQKGSRLLLLLVVSNLLLPQGVVGQSDLDRCTTFDDVQAPNYTQHTSSMRGVYYP |
| | DEIFRSDTLYLTQDLFLPFYSNVTGFHTINHTFGNPVIPFKDGIYFAATEKSNVVRGWVFGSTM |
| | NNKSQSVIIINNSTNVVIRACNFELCDNPFFAVSKPMGTQTHTMIFDNAFNCTFEYISDAFSLDV |
| | SEKSGNFKHLREFVFKNKDGFLYVYKGYQPIDVVRDLPSGFNTLKPIFKLPLGINITNFRAILTAF |
| | SPAQDIWGTSAAAYFVGYLKPTTFMLKYDENGTITDAVDCSQNPLAELKCSVKSFEIDKGIYQT |
| | SNFRVVPSGDVVRFPNITNLCPFGEVFNATKFPSVYAWERKKISNCVADYSVLYNSTFFSTFK |
| | CYGVSATKLNDLCFSNVYADSFVVKGDDVRQIAPGQTGVIADYNYKLPDDFMGCVLAWNTR |
| | NIDATSTGNYNYKYRYLRHGKLRPFERDISNVPFSPDGKPCTPPALNCYWPLNDYGFYTTTG |
| | GYQPYRVVVLSFELLNAPATVCGPKLSTDLIKNQCVNFNFNGLTGTGVLTPSSKRFQPFQQF |
| | GRDVSDFTDSVRDPKTSEILDISPCAFGGVSVITPGTNASSEVAVLYQDVNCTDVSTAIHADQL |
| | TPAWRIYSTGNNVFQTQAGCLIGAEHVDTSYECDIPIGAGICASYHTVSLLRSTSQKSIVAYTM |
| | SLGADSSIAYSNNTIAIPTNFSISITTEVMPVSMAKTSVDCNMYICGDSTECANLLLQYGSFCTQ |
| | LNRALSGIAAEQDRNTREVFAQVKQMYKTPTLKYFGGFNFSQILPDPLKPTKRSFIEDLLFNKV |
| | TLADAGFMKQYGECLGDINARDLICAQKFNGLTVLPPLLTDDMIAAYTAALVSGTATAGWTFG |
| | AGAALQIPFAMQMAYRFNGIGVTQNVLYENQKQIANQFNKAISQIQESLTTTSTALGKLQDVVN |
| | QNAQALNTLVKQLSSNFGAISSVLNDILSRLDPPEAEVQIDRLITGRLQSLQTYVTQQLIRAAE |
| | RASANLAATKMSECVLGQSKRVDFCGKGYHLMSFPQAAPHGVVFLHVTYVPSQERNFTTAP |
| | AICHEGKAYFPREGVFVFNGTSWFITQRNFFSPQIITTDNTFVSGNCDVVIGIINNTVYDPLQPE |
| | LDSFKEELDKYFKNHTSPDVDLGDISGINASVVNIQKEIDRLNEVAKNLNESLIDLQELGKYEQY |
| | IKWPWYVWLGFIAGLIAIVMVTILLCCMTSCCSCLKGACSCGSCCKFDEDDSEPVLKGVKLHY |
| | (SEQ ID NO: 539) |
| KHOSTA2_S2P | MDSKGSSQKGSRLLLLLVVSNLLLPQGVVGQQDGCATLSTKLTPTQLQVNSTRRGFYYFDDI |
| | YRSSLRVLVPGYFLPFGTNITRYQAQVINWTAIFFDNPVIPFNDGVYFAALEHSNVVRGWIFGT |
| | TLDNTTQSAIVVNNATHILISVCYFNVCKEPMFAVSNFKPYKSWVYDRANNCTFNRAYPLNIST |
| | TPEPGKFKALREQVFRYQDGFLYIYHSYESINSDTLPPGFSVLKPMLKLPLGLNITRFRVVMAM |
| | HSLTTSNFNTHSVNYFVGHLKPLTMLVEFSPNGTIIDAIDCSQDPLSELKCTTKSFSVEKGIYQT |
| | SNFRVSPSLDVVRFPNMTNICPFDQVFNKTQFPSVYAWERVRISDCVSDYTVLYNSSASFST |
| | FKCYGVSPTKLNDLCFSGVYADYFVVKGDHVHQIAPGQTGVIADYNYKLPSEFVGCILAWNT |
| | RTIDSKRGFYYRLFRHGNIRPYERDTSNVPYNAAGGTCNQPGTHNCYEPLQDYGFTSTSGV |
| | GYQPFRVVVLSFELLNAPATVCGPKQSTDLVKNKCVNFNFNGLTGTGVLTDSNKKFQPFQQF |
| | GRDSADFTDSVKDPKTLEILDITPCSYGGVSVITPGTNTSDSVAVLYQDVNCTDVPTMLHMDQ |
| | VSNDWRVYAVNTDGNMFQTQAGCLVGATYDNTSYECDIPVGAGVCAKFQTTTRAKQSSILA |
| | YTMSLGEDSNVAYSNNSIAIPTNFTISVTTEVLPVSMTKTAVDCNMYICGDSTECSSLLLQYGS |
| | FCQQLNRALAGVSVEQDKNTQDVFAQVKSIYKVSAIKDFGGFNFSQILPDPAKPSKRSFIEDLL |
| | YNKVTLADPGFMKQYGDCLGGVNARDLICAQKFNGLTVLPPLLTDDMIAAYTSALVSGTALSG |
| | FTFGAGAALQIPFAMQMAYRFNGIGVTQNVLYENQKQIANQFNKAISQIQESLTTTATALGKLQ |
| | DVINQNAIALNTLVKQLSSNFGAISSVLNDILSRLDPPEAEVQIDRLITGRLQSLQTYVTQQLIRA |
| | ADIRASANLAATKMSECVLGQSKRVDFCGKGYHLMSFPQAAPHGVVFLHVTYVPSQDKNFTT |
| | APAICHKGKAYFPREGVFVTNGTHWFVTQRNFYQPEVITTENTFESGNCDVVIGIVNNTVYDP |
| | LQPELESFKEELDKYFKNHTSPDVDFDDISGINASVVDIKKEIEHLNEIAKSLNESLIDLQELGKY |
| | EQYIKWPWYVWLGFIAGLIAIIMATIMLCCMTSCCSCLKGLCSCGSCCKFDDDHSEPVLKGVK |
| | LHY |
| | (SEQ ID NO: 540) |
| BANAL20247_ | MDSKGSSQKGSRLLLLLVVSNLLLPQGVVGQTTCVTGPTTENKQNVSSLMRGVYYPDDIYRS |
| S2P | NVNVLFTVPFLRFNSTLTWYNFWNQAYTSRVMEFGDGIYFSTVDKSNAVRGWIFGTTLDNTT |
| | QSALLINNGSLVTIQVCYFQFCANPAFLVAGGQQTSAAVYISSHNCTYSEVLSHISLDFTEDTG |
| | SFKRLREFVFKNNDGFLHIYGAYEPHVIGIGVTPSLPSTFKPLIPLWKLPLGLNITNFKVVLTFRS |
| | NSQPLQANFAVGSLKLTTFMLSFDINGTIDKAVDCSSDPLSELKCTLKSFNVSKGIYPTSNFRV |
| | LPSTEVVRFPNITNFCPFDKVFNATRFPNVYAWQRTKISDCIADYTVLYNSTSFSTFKCYGVSP |
| | SKLIDLCFTSVYADTFLIRFSEVRQIAPGETGVIADYNYKLPDDFTGCVLAWNTAQQDIGSYFY |
| | RSHRAVKLKPFERDLSSDENGVRTLSTYDFNPNVPLDYQATRVVVLSFELLNAPATVCGPKLS |
| | TQLVKNRCVNFNFNGLRGTGVLTDSDKRFQSFQQFGRDSADFTDSVRDPQTLQILDISPCSF |
| | GGVSVITPGTQTSSKVAVLYQDVNCTDVPTALGLDQISAAWRVYAIGNDVFQTQAGCLVGAE |
| | HTNISYECDIPIGAGVCASYNSPAARVGTNSIIAYAMSIGAESSIAYSNNSIAIPTNFSIQVTTEVL |
| | PVSMSKTSVDCTMYICGDSQECNKLLLQYGSFCAQLNRALSGVAVEQDKNTEAVFAQVKQIY |
| | KAPVIKDLGGFNFSQLLPDPTKPSQRSFIEDLLFDKVTLSDAGFIKQYGDCLGDIAARDLICAQK |
| | FNGLTVLPPLLTDEMIAAYTSALVSGTATAGWTFGIGAALQVPFAMQMAYRFNGIGVTQNVLY |
| | ENQKLIANQFNSAIGKIQDSLLSTASALGKLQDVVNQNAQALNTLVKQLSSNFGAISSVLNDILS |
| | RLDPPEAEVQIDRLITGRLQSLQTYVTQQLIRAAEIRASANLAATKMSECVLGQSKRVDFCGK |
| | GYHLMSFPQSAPHGVVFLHVTYVPAQEKNFTTAPAICYDGKAHFPREGVFVSNGTHWFVTQ |
| | RNFYEPQIITTDNTFVSGTCDVVIGIVNNTVYDPLQPELDSFKEELDKYFKNHTSPDVDLGDISG |
| | INASVVNIQKEIDRLNEVAKNLNESLIDLQELGKYEQYIKWPWYIWLGFIAGLIAIVMVTIMLCCM |
| | TSCCSCLKGCCSCGSCCKFDEDDSEPVLNGVKLHY |
| | (SEQ ID NO: 541) |
| MERS_S2P | MDSKGSSQKGSRLLLLLVVSNLLLPQGVVGQYVDVGPDSVKSACIEVDIQQTFFDKTWPRPID |
| | VSKADGIIYPQGRTYSNITITYQGLFPYQGDHGDMYVYSAGHATGTTPQKLFVANYSQDVKQF |
| | ANGFVVRIGAAANSTGTVIISPSTSATIRKIYPAFMLGSSVGNFSDGKMGRFFNHTLVLLPDGC |
| | GTLLRAFYCILEPRSGNHCPAGNSYTSFATYHTPATDCSDGNYNRNASLNSFKEYFNLRNCT |
| | FMYTYNITEDEILEWFGITQTAQGVHLFSSRYVDLYGGNMFQFATLPVYDTIKYYSIIPHSIRSIQ |
| | SDRKAWAAFYVYKLQPLTFLLDFSVDGYIRRAIDCGFNDLSQLHCSYESFDVESGVYSVSSFE |
| | AKPSGSVVEQAEGVECDFSPLLSGTPPQVYNFKRLVFTNCNYNLTKLLSLFSVNDFTCSQISP |
| | AAIASNCYSSLILDYFSYPLSMKSDLSVSSAGPISQFNYKQSFSNPTCLILATVPHNLTTITKPLK |
| | YSYINKCSRFLSDDRTEVPQLVNANQYSPCVSIVPSTVWEDGDYYRKQLSPLEGGGWLVAS |
| | GSTVAMTEQLQMGFGITVQYGTDTNSVCPKLEFANDTKIASQLGNCVEYSLYGVSGRGVFQN |
| | CTAVGVRQQRFVYDAYQNLVGYYSDDGNYYCLRACVSVPVSVIYDKETKTHATLFGSVACEH |
| | ISSTMSQYSRSTRSMLKRRDSTYGPLQTPVGCVLGLVNSSLFVEDCKLPLGQSLCALPDTPST |
| | LTPRSVRSVPGEMRLASIAFNHPIQVDQLNSSYFKLSIPTNFSFGVTQEYIQTTIQKVTVDCKQY |
| | VCNGFQKCEQLLREYGQFCSKINQALHGANLRQDDSVRNLFASVKSSQSSPIIPGFGGDFNL |
| | TLLEPVSISTGSRSARSAIEDLLFDKVTIADPGYMQGYDDCMQQGPASARDLICAQYVAGYKV |
| | LPPLMDVNMEAAYTSSLLGSIAGVGWTAGLSSFAAIPFAQSIFYRLNGVGITQQVLSENQKLIA |
| | NKFNQALGAMQTGFTTTNEAFHKVQDAVNNNAQALSKLASELSNTFGAISASIGDIIQRLDPPE |
| | QDAQIDRLINGRLTTLNAFVAQQLVRSESAALSAQLAKDKVNECVKAQSKRSGFCGQGTHIVS |
| | FVVNAPNGLYFMHVGYYPSNHIEVVSAYGLCDAANPTNCIAPVNGYFIKTNNTRIVDEWSYTG |
| | SSFYAPEPITSLNTKYVAPQVTYQNISTNLPPPLLGNSTGIDFQDELDEFFKNVSTSIPNFGSLT |
| | QINTTLLDLTYEMLSLQQVVKALNESYIDLKELGNYTYYNKWPWYIWLGFIAGLVALALCVFFIL |
| | CCTGCGTNCMGKLKCNRCCDRYEEYDLEPHKVHV |
| | (SEQ ID NO: 542) |
| XBB.1.5_S2P | MFVFLVLLPLVSSQCVNLITRTQSYTNSFTRGVYYPDKVFRSSVLHSTQDLFLPFFSNVTWFH |
| | AIHVSGTNGTKRFDNPALPFNDGVYFASTEKSNIIRGWIFGTTLDSKTQSLLIVNNATNVVIKVC |
| | EFQFCNDPFLDVYQKNNKSWMESEFRVYSSANNCTFEYVSQPFLMDLEGKEGNFKNLREFV |
| | FKNIDGYFKIYSKHTPINLERDLPQGFSALEPLVDLPIGINITRFQTLLALHRSYLTPVDSSSGWT |
| | AGAAAYYVGYLQPRTFLLKYNENGTITDAVDCALDPLSETKCTLKSFTVEKGIYQTSNFRVQPT |
| | ESIVRFPNITNLCPFHEVFNATTFASVYAWNRKRISNCVADYSVIYNFAPFFAFKCYGVSPTKL |
| | NDLCFTNVYADSFVIRGNEVSQIAPGQTGNIADYNYKLPDDFTGCVIAWNSNKLDSKPSGNYN |
| | YLYRLFRKSKLKPFERDISTEIYQAGNKPCNGVAGPNCYSPLQSYGFRPTYGVGHQPYRVVV |
| | LSFELLHAPATVCGPKKSTNLVKNKCVNFNFNGLTGTGVLTESNKKFLPFQQFGRDIADTTDA |
| | VRDPQTLEILDITPCSFGGVSVITPGTNTSNQVAVLYQGVNCTEVPVAIHADQLTPTWRVYST |
| | GSNVFQTRAGCLIGAEYVNNSYECDIPIGAGICASYQTQTKSHRRARSVASQSIIAYTMSLGAE |
| | NSVAYSNNSIAIPTNFTISVTTEILPVSMTKTSVDCTMYICGDSTECSNLLLQYGSFCTQLKRAL |
| | TGIAVEQDKNTQEVFAQVKQIYKTPPIKYFGGFNFSQILPDPSKPSKRSFIEDLLFNKVTLADAG |
| | FIKQYGDCLGDIAARDLICAQKFNGLTVLPPLLTDEMIAQYTSALLAGTITSGWTFGAGAALQIP |
| | FAMQMAYRFNGIGVTQNVLYENQKLIANQFNSAIGKIQDSLSSTASALGKLQDVVNHNAQALN |
| | TLVKQLSSKFGAISSVLNDILSRLDPPEAEVQIDRLITGRLQSLQTYVTQQLIRAAEIRASANLAA |
| | TKMSECVLGQSKRVDFCGKGYHLMSFPQSAPHGVVFLHVTYVPAQEKNFTTAPAICHDGKA |
| | HFPREGVFVSNGTHWFVTQRNFYEPQIITTDNTFVSGNCDVVIGIVNNTVYDPLQPELDSFKE |
| | ELDKYFKNHTSPDVDLGDISGINASVVNIQKEIDRLNEVAKNLNESLIDLQELGKYEQYIKWPW |
| | YIWLGFIAGLIAIVMVTIMLCCMTSCCSCLKGCCSCGSCCKFDEDDSEPVLKGVKLHYT |
| | (SEQ ID NO: 543) |
| Khosta2-WA1 | MDSKGSSQKGSRLLLLLVVSNLLLPQGVVGQNMTNICPFDQVFNKTQFPSVYAWERVRISDC |
| RRFN | VSDYTVLYNSSASFSTFKCYGVSPTKLNDLCFSGVYADYFVVKGDHVHQIAPGQTGVIADYNY |
| | KLPSEFVGCILAWNTRTIDSKRGFYYRLFRHGNIRPYERDTSNVPYNAAGGTCNQPGTHNCY |
| | EPLQDYGFTSTSGVGYQPFRVVVLSFENKSAPATVCGGGSGGSGNITNLCPFGEVFNATRFA |
| | SVYAWNRKRISNCVADYSVLYNSASFSTFKCYGVSPTKLNDLCFTNVYADSFVIRGDEVRQIA |
| | PGQTGKIADYNYKLPDDFTGCVIAWNSNNLDSKVGGNYNYLRRLFRKSNLKPFERDISTEIYQ |
| | AGSTPCNGVEGFNCYFPLQSYGFQPTNGVGYQPYRVVVLSFENKSAPATVCGGGSGGSGE |
| | SQVRQQFSKDIEKLLNEQVNKEMQSSNLYMSMSSWCYTHSLDGAGLFLFDHAAEEYEHAKK |
| | LIIFLNENNVPVQLTSISAPEHKFEGLTQIFQKAYEHEQHISESINNIVDHAIKSKDHATFNFLQW |
| | YVAEQHEEEVLFKDILDKIELIGNENHGLYLADQYVKGIAKSRKSGS |
| | (SEQ ID NO: 544) |
| WA1-Khosta2 | MDSKGSSQKGSRLLLLLVVSNLLLPQGVVGQNITNLCPFGEVFNATRFASVYAWNRKRISNC |
| RRFN | VADYSVLYNSASFSTFKCYGVSPTKLNDLCFTNVYADSFVIRGDEVRQIAPGQTGKIADYNYK |
| | LPDDFTGCVIAWNSNNLDSKVGGNYNYLRRLFRKSNLKPFERDISTEIYQAGSTPCNGVEGF |
| | NCYFPLQSYGFQPTNGVGYQPYRVVVLSFENKSAPATVCGGGSGGSGNMTNICPFDQVFNK |
| | TQFPSVYAWERVRISDCVSDYTVLYNSSASFSTFKCYGVSPTKLNDLCFSGVYADYFVVKGD |
| | HVHQIAPGQTGVIADYNYKLPSEFVGCILAWNTRTIDSKRGFYYRLFRHGNIRPYERDTSNVP |
| | YNAAGGTCNQPGTHNCYEPLQDYGFTSTSGVGYQPFRVVVLSFENKSAPATVCGGGSGGS |
| | GESQVRQQFSKDIEKLLNEQVNKEMQSSNLYMSMSSWCYTHSLDGAGLFLFDHAAEEYEHA |
| | KKLIIFLNENNVPVQLTSISAPEHKFEGLTQIFQKAYEHEQHISESINNIVDHAIKSKDHATFNFLQ |
| | WYVAEQHEEEVLFKDILDKIELIGNENHGLYLADQYVKGIAKSRKSGS |
| | (SEQ ID NO: 545) |
| SARS1-WA1 | MDSKGSSQKGSRLLLLLVVSNLLLPQGVVGQNITNLCPFGEVFNATKFPSVYAWERKKISNCV |
| | ADYSVLYNSTFFSTFKCYGVSATKLNDLCFSNVYADSFVVKGDDVRQIAPGQTGVIADYNYKL |
| RRFN | PDDFMGCVLAWNTRNIDATSTGNYNYKRRYLRHGKLRPFERDISNVPFSPDGKPCTPPALNC |
| | YWPLNDYGFYTTTGIGYQPYRVVVLSFENKSAPATVCGGGSGGSGNITNLCPFGEVFNATRF |
| | ASVYAWNRKRISNCVADYSVLYNSASFSTFKCYGVSPTKLNDLCFTNVYADSFVIRGDEVRQI |
| | APGQTGKIADYNYKLPDDFTGCVIAWNSNNLDSKVGGNYNYLRRLFRKSNLKPFERDISTEIY |
| | QAGSTPCNGVEGFNCYFPLQSYGFQPTNGVGYQPYRVVVLSFENKSAPATVCGGGSGGSG |
| | ESQVRQQFSKDIEKLLNEQVNKEMQSSNLYMSMSSWCYTHSLDGAGLFLFDHAAEEYEHAK |
| | KLIIFLNENNVPVQLTSISAPEHKFEGLTQIFQKAYEHEQHISESINNIVDHAIKSKDHATFNFLQ |
| | WYVAEQHEEEVLFKDILDKIELIGNENHGLYLADQYVKGIAKSRKSGS |
| | (SEQ ID NO: 546) |
| WA1-SARS1 | MDSKGSSQKGSRLLLLLVVSNLLLPQGVVGQNITNLCPFGEVFNATRFASVYAWNRKRISNC |
| RRFN | VADYSVLYNSASFSTFKCYGVSPTKLNDLCFTNVYADSFVIRGDEVRQIAPGQTGKIADYNYK |
| | LPDDFTGCVIAWNSNNLDSKVGGNYNYLRRLFRKSNLKPFERDISTEIYQAGSTPCNGVEGF |
| | NCYFPLQSYGFQPTNGVGYQPYRVVVLSFENKSAPATVCGGGSGGSGNITNLCPFGEVFNA |
| | TKFPSVYAWERKKISNCVADYSVLYNSTFFSTFKCYGVSATKLNDLCFSNVYADSFVVKGDD |
| | VRQIAPGQTGVIADYNYKLPDDFMGCVLAWNTRNIDATSTGNYNYKRRYLRHGKLRPFERDI |
| | SNVPFSPDGKPCTPPALNCYWPLNDYGFYTTTGIGYQPYRVVVLSFENKSAPATVCGGGSG |
| | GSGESQVRQQFSKDIEKLLNEQVNKEMQSSNLYMSMSSWCYTHSLDGAGLFLFDHAAEEYE |
| | HAKKLIIFLNENNVPVQLTSISAPEHKFEGLTQIFQKAYEHEQHISESINNIVDHAIKSKDHATFNF |
| | LQWYVAEQHEEEVLFKDILDKIELIGNENHGLYLADQYVKGIAKSRKSGS |
| | (SEQ ID NO: 547) |
| WA1- | MDSKGSSQKGSRLLLLLVVSNLLLPQGVVGQNITNLCPFGEVFNATRFASVYAWNRKRISNC |
| BANAL20247- | VADYSVLYNSASFSTFKCYGVSPTKLNDLCFTNVYADSFVIRGDEVRQIAPGQTGKIADYNYK |
| SARS1-WA1 RR- | LPDDFTGCVIAWNSNNLDSKVGGNYNYLRRLFRKSNLKPFERDISTEIYQAGSTPCNGVEGF |
| mosSpFN | NCYFPLQSYGFQPTNGVGYQPYRVVVLSFENKSAPATVCGGGSGGSGNITNFCPFDKVFNA |
| | TRFPNVYAWQRTKISDCIADYTVLYNSTSFSTFKCYGVSPSKLIDLCFTSVYADTFLIRFSEVR |
| | QIAPGETGVIADYNYKLPDDFTGCVLAWNTAQQDIGSYFYRSHRAVKLKPFERDLSSDENGV |
| | RTLSTYDFNPNVPLDYQATRVVVLSFENKSAPATVCGGGSGGSGSSQCVNLTTRTQLPPAY |
| | NSFTRGVYYPDKVFRSSVLHSTQDLFLPFFSNVTWFHAIHVSGTNGTKRFDNPVLPFNDGVY |
| | FASTEKSNIIRGWIFGTTLDSKTQSLLIVNNATNVVIKVCEFQFCNDPFLGVYYHKNNKSWMES |
| | EFRVYSSANNCTFEYVSQPFLMDLEGKQGNFKNLREFVFKNIDGYFKIYSKHTPINLVRDLPQ |
| | GFSALEPLVDLPIGINITRFQTLLALHRSYLTPGDSSSGWTAGAAAYYVGYLQPRTFLLKYNEN |
| | GTITDAVDCALDPLSETKCTLKSFTVEKGIYQTSNFRVQPTESIVRFPNITNLCPFGEVFNATKF |
| | PSVYAWERKKISNCVADYSVLYNSTFFSTFKCYGVSATKLNDLCFSNVYADSFVVKGDDVRQ |
| | APGQTGVIADYNYKLPDDFMGCVLAWNTRNIDATSTGNYNYKYRYLRHGKLRPFERDISNVP |
| | FSPDGKPCTPPALNCYWPLNDYGFYTTTGIGYQPYRVVVLSFELLNAPATVCGPKKSTNLVK |
| | NKCVNFNFNGLTGTGVLTESNKKFLPFQQFGRDIADTTDAVRDPQTLEILDITPCSFGGVSVIT |
| | PGTNTSNQVAVLYQDVNCTEVPVAIHADQLTPTWRVYSTGSNVFQTRAGCLIGAEHVNNSYE |
| | CDIPIGAGICASYQTQTNSPGSASSVASQSIIAYTMSLGAENSVAYSNNSIAIPTNFTISVTTEILP |
| | VSMTKTSVDCTMYICGDSTECSNLLLQYGSFCTQLNRALTGIAVEQDKNTQEVFAQVKQIYKT |
| | PPIKDFGGFNFSQILPDPSKPSKRSFIEDLLFNKVTLADAGFIKQYGDCLGDIAARDLICAQKFN |
| | GLTVLPPLLTDEMIAQYTSALLAGTITSGWTFGAGAALQIPFAMQMAYRFNGIGVTQNVLYEN |
| | QKLIANQFNSAIGKIQDSLSSTASALGKLQDVVNQNAQALNTLVKQLSSNFGAISSVLNDILSRL |
| | DPPEAEVQIDRLITGRLQSLQTYVTQQLIRAAEIRASANLAATKMSECVLGQSKRVDFCGKGY |
| | HLMSFPQSAPHGVVFLHVTYVPAQEKNFTTAPAICHDGKAHFPREGVFVSNGTHWFVTQRN |
| | FYEPQIITTDNTFVSGNCDVVIGIVNNTVYDPLQSELDSIKEELDKIHKNGSGGSGDIIKLLNEQV |
| | NKEMQSSNLYMSMSSWCYTHSLDGAGLFLFDHAAEEYEHAKKLIIFLNENNVPVQLTSISAPE |
| | HKFEGLTQIFQKAYEHEQHISESINNIVDHAIKSKDHATFNFLQWYVAEQHEEEVLFKDILDKIE |
| | LIGNENHGLYLADQYVKGIAKSRKSGS |
| | (SEQ ID NO: 548) |
| SARS1-BANAL | MDSKGSSQKGSRLLLLLVVSNLLLPQGVVGQNITNLCPFGEVFNATKFPSVYAWERKKISNC |
| 20247- | ADYSVLYNSTFFSTFKCYGVSATKLNDLCFSNVYADSFVVKGDDVRQIAPGQTGVIADYNYKL |
| Khosta2-WA1 | PDDFMGCVLAWNTRNIDATSTGNYNYKRRYLRHGKLRPFERDISNVPFSPDGKPCTPPALNC |
| RR- | YWPLNDYGFYTTTGIGYQPYRVVVLSFENKSAPATVCGGGSGGSGNITNFCPFDKVFNATRF |
| mosSpFN | PNVYAWQRTKISDCIADYTVLYNSTSFSTFKCYGVSPSKLIDLCFTSVYADTFLIRFSEVRQIAP |
| | GETGVIADYNYKLPDDFTGCVLAWNTAQQDIGSYFYRSHRAVKLKPFERDLSSDENGVRTLS |
| | TYDFNPNVPLDYQATRVVVLSFENKSAPATVCGGGSGGSGSSQCVNLTTRTQLPPAYTNSFT |
| | RGVYYPDKVFRSSVLHSTQDLFLPFFSNVTWFHAIHVSGTNGTKRFDNPVLPFNDGVYFAST |
| | EKSNIIRGWIFGTTLDSKTQSLLIVNNATNVVIKVCEFQFCNDPFLGVYYHKNNKSWMESEFR |
| | VYSSANNCTFEYVSQPFLMDLEGKQGNFKNLREFVFKNIDGYFKIYSKHTPINLVRDLPQGFS |
| | ALEPLVDLPIGINITRFQTLLALHRSYLTPGDSSSGWTAGAAAYYVGYLQPRTFLLKYNENGTIT |
| | DAVDCALDPLSETKCTLKSFTVEKGIYQTSNFRVQPTESIVRFPNMTNICPFDQVFNKTQFPSV |
| | YAWERVRISDCVSDYTVLYNSSASFSTFKCYGVSPTKLNDLCFSGVYADYFVVKGDHVHQIA |
| | PGQTGVIADYNYKLPSEFVGCILAWNTRTIDSKRGFYYRLFRHGNIRPYERDTSNVPYNAAGG |
| | TCNQPGTHNCYEPLQDYGFTSTSGVGYQPFRVVVLSFELLNAPATVCGPKKSTNLVKNKCVN |
| | FNFNGLTGTGVLTESNKKFLPFQQFGRDIADTTDAVRDPQTLEILDITPCSFGGVSVITPGTNT |
| | SNQVAVLYQDVNCTEVPVAIHADQLTPTWRVYSTGSNVFQTRAGCLIGAEHVNNSYECDIPIG |
| | AGICASYQTQTNSPGSASSVASQSIIAYTMSLGAENSVAYSNNSIAIPTNFTISVTTEILPVSMTK |
| | TSVDCTMYICGDSTECSNLLLQYGSFCTQLNRALTGIAVEQDKNTQEVFAQVKQIYKTPPIKDF |
| | GGFNFSQILPDPSKPSKRSFIEDLLFNKVTLADAGFIKQYGDCLGDIAARDLICAQKFNGLTVLP |
| | PLLTDEMIAQYTSALLAGTITSGWTFGAGAALQIPFAMQMAYRFNGIGVTQNVLYENQKLIANQ |
| | FNSAIGKIQDSLSSTASALGKLQDVVNQNAQALNTLVKQLSSNFGAISSVLNDILSRLDPPEAE |
| | VQIDRLITGRLQSLQTYVTQQLIRAAEIRASANLAATKMSECVLGQSKRVDFCGKGYHLMSFP |
| | QSAPHGVVFLHVTYVPAQEKNFTTAPAICHDGKAHFPREGVFVSNGTHWFVTQRNFYEPQII |
| | TTDNTFVSGNCDVVIGIVNNTVYDPLQSELDSIKEELDKIHKNGSGGSGDIIKLLNEQVNKEMQ |
| | SSNLYMSMSSWCYTHSLDGAGLFLFDHAAEEYEHAKKLIIFLNENNVPVQLTSISAPEHKFEG |
| | LTQIFQKAYEHEQHISESINNIVDHAIKSKDHATFNFLQWYVAEQHEEEVLFKDILDKIELIGNEN |
| | HGLYLADQYVKGIAKSRKSGS |
| | (SEQ ID NO: 549) |
| Khosta2- | MDSKGSSQKGSRLLLLLVVSNLLLPQGVVGQNMTNICPFDQVFNKTQFPSVYAWERVRISDC |
| BANAL20247- | VSDYTVLYNSSASFSTFKCYGVSPTKLNDLCFSGVYADYFVVKGDHVHQIAPGQTGVIADYNY |
| SARS1 | KLPSEFVGCILAWNTRTIDSKRGFYYRLFRHGNIRPYERDTSNVPYNAAGGTCNQPGTHNCY |
| WA1 RR- | EPLQDYGFTSTSGVGYQPFRVVVLSFENKSAPATVCGGGSGGSGNITNFCPFDKVFNATRFP |
| mosSpFN | NVYAWQRTKISDCIADYTVLYNSTSFSTFKCYGVSPSKLIDLCFTSVYADTFLIRFSEVRQIAPG |
| | ETGVIADYNYKLPDDFTGCVLAWNTAQQDIGSYFYRSHRAVKLKPFERDLSSDENGVRTLST |
| | YDFNPNVPLDYQATRVVVLSFENKSAPATVCGGGSGGSGSSQCVNLTTRTQLPPAYTNSFT |
| | RGVYYPDKVFRSSVLHSTQDLFLPFFSNVTWFHAIHVSGTNGTKRFDNPVLPFNDGVYFAST |
| | EKSNIIRGWIFGTTLDSKTQSLLIVNNATNVVIKVCEFQFCNDPFLGVYYHKNNKSWMESEFR |
| | VYSSANNCTFEYVSQPFLMDLEGKQGNFKNLREFVFKNIDGYFKIYSKHTPINLVRDLPQGFS |
| | ALEPLVDLPIGINITRFQTLLALHRSYLTPGDSSSGWTAGAAAYYVGYLQPRTFLLKYNENGTIT |
| | DAVDCALDPLSETKCTLKSFTVEKGIYQTSNFRVQPTESIVRFPNITNLCPFGEVFNATKFPSV |
| | YAWERKKISNCVADYSVLYNSTFFSTFKCYGVSATKLNDLCFSNVYADSFVVKGDDVRQIAP |
| | GQTGVIADYNYKLPDDFMGCVLAWNTRNIDATSTGNYNYKYRYLRHGKLRPFERDISNVPFS |
| | PDGKPCTPPALNCYWPLNDYGFYTTTGIGYQPYRVVVLSFELLNAPATVCGPKKSTNLVKNK |
| | CVNFNFNGLTGTGVLTESNKKFLPFQQFGRDIADTTDAVRDPQTLEILDITPCSFGGVSVITPG |
| | TNTSNQVAVLYQDVNCTEVPVAIHADQLTPTWRVYSTGSNVFQTRAGCLIGAEHVNNSYECD |
| | IPIGAGICASYQTQTNSPGSASSVASQSIIAYTMSLGAENSVAYSNNSIAIPTNFTISVTTEILPVS |
| | MTKTSVDCTMYICGDSTECSNLLLQYGSFCTQLNRALTGIAVEQDKNTQEVFAQVKQIYKTPP |
| | KDFGGFNFSQILPDPSKPSKRSFIEDLLFNKVTLADAGFIKQYGDCLGDIAARDLICAQKFNGLT |
| | VLPPLLTDEMIAQYTSALLAGTITSGWTFGAGAALQIPFAMQMAYRENGIGVTQNVLYENQKLI |
| | ANQFNSAIGKIQDSLSSTASALGKLQDVVNQNAQALNTLVKQLSSNFGAISSVLNDILSRLDPP |
| | EAEVQIDRLITGRLQSLQTYVTQQLIRAAEIRASANLAATKMSECVLGQSKRVDFCGKGYHLM |
| | SFPQSAPHGVVFLHVTYVPAQEKNFTTAPAICHDGKAHFPREGVFVSNGTHWFVTQRNFYE |
| | PQIITTDNTFVSGNCDVVIGIVNNTVYDPLQSELDSIKEELDKIHKNGSGGSGDIIKLLNEQVNKE |
| | MQSSNLYMSMSSWCYTHSLDGAGLFLFDHAAEEYEHAKKLIIFLNENNVPVQLTSISAPEHKF |
| | EGLTQIFQKAYEHEQHISESINNIVDHAIKSKDHATFNFLQWYVAEQHEEEVLFKDILDKIELIGN |
| | ENHGLYLADQYVKGIAKSRKSGS |
| | (SEQ ID NO: 550) |
| XBB.1.5 SpFN | MDSKGSSQKGSRLLLLLVVSNLLLPQGVVGSSQCVNLITRTQSYTNSFTRGVYYPDKVFRSS |
| | VLHSTQDLFLPFFSNVTWFHAIHVSGTNGTKRFDNPALPFNDGVYFASTEKSNIIRGWIFGTTI |
| | DSKTQSLLIVNNATNVVIKVCEFQFCNDPFLDVYQKNNKSWMESEFRVYSSANNCTFEYVSQ |
| | PFLMDLEGKEGNFKNLREFVFKNIDGYFKIYSKHTPINLERDLPQGFSALEPLVDLPIGINITRFQ |
| | TLLALHRSYLTPVDSSSGWTAGAAAYYVGYLQPRTFLLKYNENGTITDAVDCALDPLSETKCT |
| | LKSFTVEKGIYQTSNFRVQPTESIVRFPNITNLCPFHEVFNATTFASVYAWNRKRISNCVADYS |
| | VIYNFAPFFAFKCYGVSPTKLNDLCFTNVYADSFVIRGNEVSQIAPGQTGNIADYNYKLPDDFT |
| | GCVIAWNSNKLDSKPSGNYNYLYRLFRKSKLKPFERDISTEIYQAGNKPCNGVAGPNCYSPL |
| | QSYGFRPTYGVGHQPYRVVVLSFELLHAPATVCGPKKSTNLVKNKCVNFNFNGLTGTGVLTE |
| | SNKKFLPFQQFGRDIADTTDAVRDPQTLEILDITPCSFGGVSVITPGTNTSNQVAVLYQGVNCT |
| | EVPVAIHADQLTPTWRVYSTGSNVFQTRAGCLIGAEYVNNSYECDIPIGAGICASYQTQTKSP |
| | GSASSVASQSIIAYTMSLGAENSVAYSNNSIAIPTNFTISVTTEILPVSMTKTSVDCTMYICGDST |
| | ECSNLLLQYGSFCTQLKRALTGIAVEQDKNTQEVFAQVKQIYKTPPIKYFGGFNFSQILPDPSK |
| | PSKRSFIEDLLFNKVTLADAGFIKQYGDCLGDIAARDLICAQKFNGLTVLPPLLTDEMIAQYTSA |
| | LLAGTITSGWTFGAGAALQIPFAMQMAYRFNGIGVTQNVLYENQKLIANQFNSAIGKIQDSLSS |
| | TASALGKLQDVVNHNAQALNTLVKQLSSKFGAISSVLNDILSRLDPPEAEVQIDRLITGRLQSLQ |
| | TYVTQQLIRAAEIRASANLAATKMSECVLGQSKRVDFCGKGYHLMSFPQSAPHGVVFLHVTY |
| | VPAQEKNFTTAPAICHDGKAHFPREGVFVSNGTHWFVTQRNFYEPQIITTDNTFVSGNCDVVI |
| | GIVNNTVYDPLQSELDSIKEELDKIHKNGSGGSGDIIKLLNEQVNKEMQSSNLYMSMSSWCYT |
| | HSLDGAGLFLFDHAAEEYEHAKKLIIFLNENNVPVQLTSISAPEHKFEGLTQIFQKAYEHEQHIS |
| | ESINNIVDHAIKSKDHATFNFLQWYVAEQHEEEVLFKDILDKIELIGNENHGLYLADQYVKGIAK |
| | SRKSGS |
| | (SEQ ID NO: 551) |
| Strain Design | Nucleic Acid Sequence |
| empty FN | ATGGACTCCAAAGGGTCTAGCCAGAAGGGGTCCCGCCTCCTGCTGCTGCTGGTGGTGAGCAACCTGCTGCTCCCTCAGGGCGTGGTGGGACAGGAGTCCCAGGTGAGACAGCAGTTTAGCAA |
| | GGATATCGAGAAGCTGCTGAATGAGCAGGTCAACAAAGAGATGCAGTCTAGCAACCTGTACATGAGCATGAGCTCCTGGTGCTATACCCACAGCCTCGACGGCGCTGGCCTGTTCCTGTTCG |
| | ACCACGCAGCCGAGGAGTACGAGCATGCCAAGAAGCTGATTATTTTCCTGAATGAGAACAACGTGCCAGTCCAGCTGACATCCATTTCCGCTCCCGAACACAAGTTCGAGGGCCTGACCCAG |
| | ATCTTCCAGAAGGCATACGAGCACGAGCAGCACATCTCCGAAAGCATCAATAATATCGTGGATCACGCCATCAAGTCCAAAGATCACGCCACCTTTAACTTCCTGCAGTGGTACGTGGCCGA |
| | GCAGCACGAGGAGGAGGTGCTGTTCAAAGATATTCTGGACAAGATCGAGCTGATCGGCAATGAAAACCATGGCCTGTATCTGGCCGATCAGTACGTGAAGGGAATTGCAAAATCCAGAAAAA |
| | GTGGCTCCTAA (SEQ ID NO: 552) |
| WA-1 RFN | ATGGACAGCAAGGGATCTAGCCAGAAGGGCAGCCGGCTGCTGCTGCTGCTGGTGGTGAGCAACCTGCTGCTGCCTCAGGGCGTGGTGGGCCAAAACATCACCAACCTGTGCCCTTTCGGCGA |
| | AGTGTTCAATGCCACCAGATTCGCCTCCGTGTACGCCTGGAACCGGAAGCGGATCAGCAATTGCGTGGCCGACTACTCTGTGCTGTACAATAGCGCCTCTTTCAGCACCTTCAAGTGCTACG |
| | GCGTGAGCCCCACCAAGCTGAACGACCTGTGCTTCACCAACGTGTACGCCGACAGCTTCGTGATCCGGGGCGACGAGGTGCGGCAGATTGCCCCTGGCCAGACAGGAAAGATCGCTGACTAC |
| | AATTACAAGCTGCCCGACGATTTTACCGGCTGCGTGATCGCCTGGAACAGCAATAACCTGGACTCCAAGGTGGGCGGCAACTACAATTACCTGCGGAGACTGTTCAGAAAGAGCAACCTGAA |
| | GCCCTTCGAGAGAGACATCTCCACCGAGATCTACCAGGCCGGCAGCACCCCCTGCAATGGCGTGGAGGGCTTCAATTGTTACTTCCCCCTGCAGAGCTATGGCTTCCAGCCTACCAACGGCG |
| | TGGGCTACCAGCCCTACAGAGTGGTGGTGCTGAGCTTCGAGAACAAGAGCGCCCCCGCCACAGTGTGTGGAGGCGGCTCTGGCGGCTCCGGCGAGAGCCAGGTGAGACAGCAGTTCAGCAAG |
| | GACATCGAGAAGCTGCTGAATGAACAGGTGAATAAGGAAATGCAGAGCTCTAACCTGTACATGAGCATGAGCTCTTGGTGCTACACCCACTCCCTGGATGGCGCCGGCCTGTTCCTGTTTGA |
| | CCACGCCGCCGAGGAGTACGAGCACGCTAAGAAGCTGATCATCTTCCTGAATGAGAATAACGTGCCTGTGCAGCTGACCAGCATCAGCGCCCCCGAGCACAAATTCGAGGGCCTGACCCAGA |
| | TCTTCCAGAAGGCCTACGAGCACGAGCAGCACATCTCCGAGAGCATCAATAACATCGTGGACCACGCCATCAAGAGCAAGGACCACGCCACCTTCAATTTCCTGCAGTGGTACGTGGCCGAG |
| | CAGCACGAGGAGGAGGTGCTGTTTAAGGACATCCTGGACAAGATTGAACTGATCGGCAATGAGAATCACGGCCTGTACCTGGCCGACCAGTACGTGAAGGGCATCGCCAAAAGCAGAAAGAG |
| | CGGCTCTTAA |
| | (SEQ ID NO: 553) |
| Beta RFN | ATGGATAGCAAAGGCAGCAGCCAGAAGGGGAGCAGACTGCTGCTGCTGCTGGTGGTGTCTAACCTGCTGCTGCCTCAGGGCGTGGTGGGCCAGAATATTACCAACCTGTGCCCATTCGGCGA |
| | GGTGTTCAATGCCACCCGGTTCGCCTCCGTGTACGCCTGGAATCGGAAGAGAATCTCCAACTGCGTGGCCGACTACTCCGTGCTGTACAATAGCGCCAGCTTCAGCACCTTCAAGTGCTACG |
| | GCGTGTCCCCCACCAAGCTGAATGACCTGTGCTTCACCAACGTGTACGCCGACAGCTTCGTGATCAGAGGCGACGAGGTGAGACAGATCGCCCCTGGCCAGACCGGCAATATCGCCGACTAC |
| | AATTACAAGCTGCCCGACGACTTCACCGGCTGCGTGATCGCCTGGAACAGCAATAATCTGGACTCCAAGGTGGGCGGCAATTACAATTACCTGAGACGGCTGTTCAGAAAGAGCAACCTGAA |
| | GCCCTTCGAGAGAGACATCAGCACCGAGATCTACCAGGCCGGCAGCACCCCCTGCAACGGCGTGAAGGGCTTCAATTGCTACTTCCCTCTGCAGAGCTACGGCTTCCAGCCCACCTACGGCG |
| | TGGGCTACCAGCCTTACAGAGTGGTGGTGCTGAGCTTCGAGAATAAGAGCGCCCCCGCCACCGTGTGCGGCGGAGGCAGCGGCGGCAGCGGCGAGAGCCAGGTGAGACAGCAGTTCTCCAAG |
| | GACATCGAAAAGCTGCTGAACGAGCAGGTGAACAAGGAGATGCAGAGCAGCAACCTGTACATGAGCATGAGCTCCTGGTGCTACACCCATAGCCTGGACGGCGCCGGCCTGTTCCTGTTCGA |
| | CCACGCCGCCGAGGAGTACGAGCACGCCAAGAAGCTGATCATCTTCCTGAATGAAAATAACGTGCCCGTGCAGCTGACCAGCATCAGCGCCCCCGAGCACAAGTTCGAGGGCCTGACCCAGA |
| | TCTTCCAGAAGGCCTACGAGCACGAGCAGCACATCAGCGAGAGCATCAATAATATCGTGGACCACGCCATCAAGAGCAAGGATCACGCCACCTTCAACTTTCTGCAGTGGTACGTGGCCGAG |
| | CAGCACGAGGAGGAGGTGCTGTTCAAGGACATCCTGGACAAGATCGAGCTGATCGGCAACGAGAACCACGGCCTGTACCTGGCCGATCAGTACGTGAAGGGCATCGCCAAGTCCAGAAAGAG |
| | CGGCAGCTAA(SEQ ID NO: 554) |
| BQ1.1 RFN | ATGGACTCTAAGGGCAGCAGCCAGAAAGGCAGCAGGCTGCTGCTGCTGCTCGTGGTCAGCAACCTGCTGCTGCCCCAGGGCGTTGTGGGCCAGAACATCACCAACCTTTGCCCATTTGACGA |
| | GGTGTTTAACGCCACTACTTTCGCTAGCGTCTATGCCTGGAACAGAAAGAGGATTAGCAATTGCGTTGCAGACTATAGCGTGCTGTATAATTTCGCCCCATTCTTTGCTTTCAAGTGCTACG |
| | GCGTCTCTCCCACCAAGCTGAACGACCTGTGCTTTACAAACGTGTATGCTGACAGCTTCGTGATCCGCGGAATGAAGTGTCCCAGATTGCCCCAGGCCAGACTGGAAATATCGCTGACTACA |
| | ATTACAAGCTGCCCGATGATTTCACTGGCTGCGTGATTGCCTGGAACAGTAACAAGCTGGACTCCACCGTGGGGGGAAATTATAATTATAGAAGGCGCCTGTTCAGGAAGTCAAAGCTGAAA |
| | CCTTTCGAGAGGGACATCTCTACCGAGATCTACCAGGCTGGAAACAAGCCCTGCAATGGCGTGGCCGGCGTGAATTGTTATTTCCCACTCCAGAGCTACGGGTTCCGGCCAACTTATGGAGT |
| | GGGGCACCAACCTTACAGGGTGGTGGTGCTGAGCTTCGAAAACAAGTCCGCTCCTGCAACAGTGTGCGGCGGCGGATCCGGCGGCAGCGGCGAGAGCCAGGTCAGACAGCAGTTTAGCAAGG |
| | ATATCGAGAAACTGCTCAACGAGCAGGTGAATAAGGAGATGCAGTCTTCTAACCTGTATATGAGCATGAGCTCTTGGTGTTATACTCACTCTCTGGACGGAGCTGGCCTGTTCCTGTTCGAC |
| | CACGCCGCTGAGGAGTACGAGCACGCAAAGAAGCTGATCATCTTCCTGAACGAAAACAATGTTCCAGTGCAGCTGACTTCCATTAGCGCCCCCGAGCACAAGTTCGAGGGACTGACACAGAT |
| | TTTTCAGAAAGCCTACGAGCACGAACAGCACATCAGCGAGTCAATTAATAACATCGTGGACCATGCCATTAAGTCTAAGGACCACGCTACCTTTAACTTCCTGCAGTGGTATGTGGCAGAAC |
| | AGCACGAGGAAGAGGTGCTCTTTAAAGATATCCTGGACAAGATCGAGCTGATCGGCAATGAGAATCACGGCCTGTATCTGGCCGATCAGTACGTGAAAGGGATTGCTAAGTCTCGCAAATCC |
| | GGCAGCTAA (SEQ ID NO: 555) |
| SARS-CoV-1 | ATGGATTCCAAAGGCTCCAGCCAGAAGGGAAGCAGACTGCTGCTGCTGCTGGTGGTGTCAAACCTGCTCCTGCCTCAGGGAGTGGTGGGGCAAAATATTACCAACCTGTGTCCATTTGGGGA |
| RFN | GGTGTTTAACGCCACCAAGTTCCCTAGCGTGTATGCCTGGGAAAGAAAAAAGATTTCCAACTGCGTGGCAGACTATAGCGTGCTGTACAATTCTACCTTCTTCTCTACCTTCAAGTGCTATG |
| | GCGTTAGCGCAACTAAGCTGAACGACCTCTGCTTCTCCAACGTGTACGCCGATAGTTTCGTGGTCAAAGGCGATGATGTGCGCCAGATCGCCCCAGGCCAGACCGGAGTGATCGCCGACTAT |
| | AACTATAAGCTGCCTGACGACTTTATGGGCTGTGTGCTGGCTTGGAACACACGGAACATCGACGCCACCAGCACCGGGAATTATAACTACAAGAGACGGTATCTGAGACACGGAAAGCTCAG |
| | ACCATTTGAGCGGGATATCAGCAACGTGCCCTTTAGTCCCGACGGGAAGCCTTGCACCCCTCCTGCACTGAACTGCTACTGGCCTCTGAACGACTACGGCTTCTATACAACCACAGGAATCG |
| | GATACCAGCCATATAGGGTCGTGGTGCTGAGCTTCGAGAATAAGAGTGCACCAGCTACCGTGTGCGGCGGAGGAAGCGGGGGCAGCGGCGAATCACAGGTGAGGCAGCAGTTTTCCAAGGAC |
| | ATTGAGAAGCTGCTGAACGAACAGGTGAACAAGGAGATGCAGTCTTCTAACCTGTACATGTCCATGTCCTCTTGGTGTTACACTCACTCCCTGGATGGAGCCGGCCTGTTCCTCTTTGACCA |
| | CGCCGCTGAGGAATACGAGCACGCTAAAAAGCTGATCATCTTCCTGAACGAGAACAATGTGCCAGTTCAGCTGACCAGCATCTCTGCACCCGAACACAAATTTGAGGGCCTGACCCAGATCT |
| | TCCAGAAGGCCTACGAGCACGAGCAACATATTTCCGAGTCCATCAATAATATCGTGGACCACGCCATCAAGTCCAAGGATCACGCCACATTCAACTTCCTGCAGTGGTACGTGGCTGAGCAG |
| | CACGAGGAAGAGGTGCTCTTTAAGGACATCCTGGACAAGATTGAGCTCATCGGAAACGAAAACCACGGCCTGTATCTGGCCGACCAGTATGTGAAGGGCATTGCTAAGTCTCGGAAATCCGG |
| | GTCTTAA (SEQ ID NO: 556) |
| Khosta-2 RFN | ATGGACAGCAAGGGCAGCAGCCAGAAGGGCAGCAGACTGCTGCTGCTGCTGGTGGTGAGCAACCTGCTGCTGCCTCAGGGCGTGGTGGGCCAGAATATGACCAATATTTGCCCATTCGACCA |
| | GGTGTTCAATAAGACCCAGTTCCCCAGCGTGTACGCCTGGGAGCGCGTGAGAATCAGCGACTGCGTGTCCGATTACACCGTGCTGTACAATAGCAGCGCCAGCTTCAGCACCTTCAAGTGTT |
| | ACGGCGTGAGCCCCACAAAGCTGAATGACCTGTGCTTCAGCGGCGTGTACGCCGACTACTTCGTGGTGAAGGGCGACCACGTGCACCAGATCGCCCCCGGCCAGACAGGCGTGATCGCCGAT |
| | TACAATTACAAGCTGCCCAGCGAGTTCGTGGGCTGTATCCTGGCCTGGAATACCAGAACCATCGATAGCAAGCGGGGCTTCTACTACAGACTGTTCAGACACGGCAACATCCGGCCCTACGA |
| | GAGAGACACCAGCAATGTGCCATACAATGCCGCCGGCGGCACATGCAATCAGCCTGGCACACACAATTGCTACGAGCCCCTGCAGGACTACGGATTCACCAGCACCAGCGGCGTGGGCTACC |
| | AGCCTTTCAGAGTGGTGGTGCTGAGCTTCGAGAATAAGTCTGCCCCTGCCACCGTGTGCGGAGGCGGCTCCGGAGGCTCTGGAGAGAGCCAGGTGCGGCAGCAGTTCAGCAAGGACATCGAG |
| | AAGCTGCTGAATGAACAGGTGAATAAGGAGATGCAGAGCAGCAACCTGTACATGAGCATGAGCAGCTGGTGCTACACCCACAGCCTGGACGGCGCCGGACTGTTCCTGTTCGATCACGCCGC |
| | CGAGGAATACGAGCACGCCAAGAAGCTGATCATCTTCCTGAATGAGAACAATGTGCCCGTGCAGCTGACCAGCATCTCTGCCCCTGAGCACAAGTTCGAGGGCCTGACCCAGATCTTCCAGA |
| | AGGCCTACGAGCACGAGCAGCACATCAGCGAGAGCATTAACAATATCGTGGACCACGCCATCAAGAGCAAGGACCACGCCACCTTCAATTTCCTGCAGTGGTACGTGGCCGAGCAGCACGAG |
| | GAGGAAGTGCTGTTCAAGGACATCCTGGACAAGATCGAGCTGATCGGCAATGAGAATCACGGCCTGTACCTGGCCGACCAGTACGTGAAAGGCATCGCCAAGAGCAGAAAGAGCGGCAGCTA |
| | A (SEQ ID NO: 557) |
| BANAL20-247 | ATGGATTCAAAAGGATCCAGCCAGAAAGGATCCCGCCTGCTGCTGCTGCTGGTGGTGAGCAACCTGCTGCTGCCTCAGGGGGTGGTGGGCCAGAACATCACGAACTTCTGCCCATTTGATAA |
| RFN | AGTGTTCAACGCCACACGCTTCCCAAACGTGTACGCCTGGCAGCGCACCAAGATCTCCGATTGCATTGCCGACTACACCGTGCTGTATAACTCAACCAGCTTTTCCACTTTCAAGTGTTACG |
| | GAGTGAGCCCCAGCAAGCTGATCGACCTGTGTTTCACCAGCGTTTACGCTGACACTTTCCTGATTCGGTTCTCTGAGGTCCGCCAGATCGCCCCCGGGGAAACCGGAGTGATCGCTGACTAT |
| | AACTACAAGCTGCCAGATGACTTCACCGGATGTGTGCTGGCTTGGAATACTGCTCAGCAGGACATTGGCAGCTACTTCTATCGGTCCCACAGAGCCGTGAAGCTGAAGCCCTTCGAGAGGGA |
| | CCTGTCTTCTGACGAGAACGGGGTGAGGACCCTGTCCACCTACGACTTTAATCCTAACGTGCCTCTCGACTACCAGGCCACCCGCGTGGTGGTGCTGAGCTTCGAGAACAAGTCTGCCCCA |
| | GCCACTGTGTGTGGAGGGGGATCAGGGGGATCTGGCGAGAGCCAGGTGAGACAGCAATTCTCCAAGGATATTGAGAAGCTGCTCAACGAGCAGGTGAACAAGGAAATGCAGTCAAGCAACCT |
| | GTATATGAGCATGAGCAGTTGGTGCTACACACATTCTCTGGACGGGGCCGGCCTGTTCCTGTTCGACCACGCCGCCGAGGAATACGAGCACGCCAAGAAGCTGATTATCTTTCTGAACGAGA |
| | ACAACGTCCCAGTGCAGCTGACCAGCATCAGCGCCCCCGAACATAAATTCGAGGGCCTGACTCAGATTTTTCAGAAGGCTTATGAGCACGAGCAGCATATCAGCGAGTCTATCAACAACATC |
| | GTGGACCACGCCATCAAGAGCAAGGATCACGCAACCTTCAACTTCCTGCAGTGGTATGTGGCAGAACAGCATGAGGAAGAGGTGCTGTTCAAGGATATCCTGGACAAGATCGAGCTGATCGG |
| | CAACGAGAACCACGGACTGTATCTGGCAGACCAGTATGTGAAAGGAATCGCCAAGAGCAGAAAGAGTGGCTCCTAA (SEQ ID NO: 558) |
| MERS RFN | ATGGATAGCAAGGGCTCCAGCCAGAAGGGCAGCCGGCTGCTGCTGCTGCTGGTGGTGAGCAACCTGCTGCTGCCTCAGGGCGTGGTGGGCCAGGAGGGAGTGGAGTGCGACTTCAGCCCTCT |
| | GCTGAGCGGAACCCCACCACAGGTGTACAATTTCAAGAGACTGGTGTTCACCAATTGCAATTACAATCTGACCAAGCTGCTGAGCCTGTTCAGCGTGAATGACTTCACCTGCAGCCAGATCA |
| | GCCCTGCCGCCATCGCCAGCAATTGCTACAGCAGCCTGATTCTGGACTACTTCAGCTACCCCCTGAGCATGAAATCCGACCTGTCTGTGAGCAGCGCCGGCCCTATCAGCCAGTTCAATTAC |
| | AAACAGAGCTTCAGCAACCCTACCTGCCTGATCCTGGCCACCGTGCCTCACAACCTGACCACCATCACCAAGCCTCTGAAGTATAGCTACATCAACAAGTGCAGCCGGCTGCTGTCCGATGA |
| | CCGGACCGAGGTGCCCCAGCTGGTGAATGCCAATCAGTACTCCCCCTGCGTGAGCATCGTGCCATCCACCGTGGCCGAGGATGGCGACTACTACCGGAAGCAGCTGAGCCCCCTGGAAGGAG |
| | GCGGCTGGCTGGTGGCCTCTGGCAGCACAGTGGCCATGACCGAGCAGCTGCAGATGGGCTTCGGCATCACCGTGCAGAATGGCACAGATACCAATAGCGTGTGCGGCGGCGGCTCTGGAGGC |
| | AGCGGCGAGAGCCAGGTGCGGCAGCAGTTCAGCAAGGACATCGAGAACTGAATGAGAATAACGTGCCTGTGCAGCTGACCAGCATCAGCGCCCCCGAGCACAAGTTCGAGGGCCTGACACAG |
| | ATCTTCCAGAAGGCCTACGAACATGAGCAGCACATCAGCGAGAGCATCAATAATATCGTGGACCACGCCATCAAGTCCAAGGACCACGCCACCTTCAACTTTCTGCAGTGGTACGTGGCCGA |
| | GCAGCACGAGGAGGAGGTGCTGTTCAAGGACATCCTGGATAAGATCGAGCTGATTGGCAATGAGAACCACGGCCTGTACCTGGCTGATCAGTACGTGAAGGGCATCGCCAAGAGCCGGAAGT |
| | CCGGCAGCTAA (SEQ ID NO: 559) |
| WA-1 SpFN | ATGGACAGCAAGGGAAGCTCCCAGAAAGGATCTCGACTGCTGCTGCTGCTGGTGGTGTCCAACCTGCTGCTGCCACAGGGCGTCGTGGGCCAAAGCAGCCAGTGTGTGAACCTCACTACTAG |
| | AACCCAGCTGCCCCCAGCCTACACCAATTCCTTTACAAGGGGGGTGTACTATCCCGATAAGGTGTTCAGAAGCTCTGTGCTGCATTCTACACAGGACCTCTTTCTGCCCTTTTTCAGCAACG |
| | TGACATGGTTTCACGCCATCCACGTGAGCGGGACAAATGGGACAAAAAGGTTTGATAACCCCGTGCTGCCGTTTAACGACGGGGTGTATTTCGCAAGCACCGAGAAGAGTAACATTATCCGG |
| | GGATGGATCTTCGGAACTACACTGGACTCCAAGACACAGTCTCTGCTGATCGTGAACAATGCAACAAACGTGGTCATTAAGGTGTGCGAGTTCCAGTTCTGCAACGATCCATTCCTGGGAGT |
| | GTATTACCATAAGAACAACAAGAGCTGGATGGAGTCAGAGTTCAGGGTGTACTCCAGCGCCAACAATTGTACTTTTGAGTATGTGTCTCAGCCATTCCTGATGGACCTGGAGGGCAAACAGG |
| | GCAACTTCAAAAATCTGAGGGAGTTCGTGTTTAAGAACATCGATGGGTATTTCAAAATCTACTCCAAGCACACACCTATTAACCTGGTGCGCGATCTGCCTCAGGGCTTTTCCGCTCTGGAG |
| | CCACTGGTGGACCTGCCAATCGGAATTAACATTACCAGATTCCAGACTCTGCTGGCCCTCCACCGGAGCTACCTCACACCAGGAGACAGCTCCAGTGGCTGGACCGCCGGAGCCGCTGCCTA |
| | CTATGTGGGCTATCTGCAGCCTAGAACTTTCCTGCTGAAGTATAATGAAAATGGCACAATCACCGACGCAGTCGATTGCGCTCTGGATCCTCTGTCTGAGACTAAGTGCACCCTGAAGTCCT |
| | TCACCGTCGAAAAGGGCATCTATCAGACAAGCAATTTCCGGGTGCAGCCCACAGAGAGCATCGTGAGATTCCCCAATATCACTAACCTGTGCCCATTCGGCGAGGTGTTTAACGCTACCCGC |
| | TTCGCCTCCGTCTACGCATGGAACAGAAAAAGGATTTCTAACTGTGTGGCTGACTACTCTGTGCTGTATAATTCTGCTTCCTTCTCAACCTTTAAATGCTACGGGGTGAGCCCCACCAAACT |
| | GAACGACCTGTGTTTCACCAACGTGTATGCTGATAGCTTCGTGATCAGGGGAGATGAGGTTCGGCAGATCGCACCAGGCCAGACAGGCAAAATCGCCGATTACAATTACAAGCTGCCCGATG |
| | ACTTTACCGGCTGTGTGATCGCATGGAACAGCAACAACCTGGACAGCAAGGTGGGCGGCAATTACAATTACCTGTATCGCCTGTTTAGGAAGTCCAACCTGAAGCCTTTTGAGCGGGACATC |
| | TCTACTGAGATCTATCAAGCCGGGAGCACTCCATGTAACGGCGTGGAGGGGTTCAATTGTTATTTCCCTCTGCAGAGCTATGGCTTCCAGCCAACTAACGGGGTGGGCTACCAGCCCTATCG |
| | GGTCGTGGTGCTGAGCTTCGAACTGCTGCACGCTCCAGCCACAGTCTGCGGACCCAAGAAGTCCACAAATCTCGTGAAAAATAAGTGCGTGAACTTCAATTTCAACGGACTGACCGGAACAG |
| | GCGTGCTGACTGAGAGCAACAAGAAGTTCCTGCCATTTCAGCAGTTCGGCCGGGATATCGCAGATACCACCGACGCCGTCCGCGACCCTCAGACACTGGAAATCCTGGATATTACCCCATGC |
| | AGCTTCGGAGGCGTGTCTGTCATTACACCAGGCACAAACACATCCAACCAGGTGGCCGTGCTGTATCAGGATGTCAACTGCACAGAGGTGCCCGTGGCCATCCACGCCGACCAGCTGACACC |
| | CACCTGGAGAGTGTACTCTACAGGATCAAACGTCTTTCAGACCCGGGCAGGATGTCTGATTGGGGCAGAGCATGTGAACAACTCCTATGAGTGCGACATTCCAATCGGGGCTGGGATCTGCG |
| | CTTCCTATCAGACTCAGACAAATTCCCCAGGCTCCGCCAGCAGCGTGGCCTCTCAGAGCATTATCGCATATACCATGTCTCTGGGGGCCGAGAACAGCGTGGCCTACTCTAATAATAGCATC |
| | GCCATTCCAACTAACTTTACTATTAGCGTCACCACAGAGATTCTGCCCGTGAGCATGACAAAAACCTCTGTGGATTGCACTATGTATATTTGCGGGGACTCCACAGAGTGCTCCAACCTGCT |
| | GCTGCAGTACGGCTCCTTTTGCACCCAGCTGAACCGCGCACTGACAGGAATTGCTGTGGAGCAGGATAAGAATACCCAGGAGGTCTTCGCTCAGGTGAAGCAGATCTACAAGACTCCACCCA |
| | TCAAGGACTTCGGCGGATTTAATTTCTCCCAGATCCTGCCAGATCCTTCCAAGCCCAGCAAGCGCAGCTTTATCGAGGACCTGCTCTTTAATAAGGTGACCCTGGCAGACGCTGGATTCATC |
| | AAGCAGTATGGAGATTGCCTGGGGGACATCGCAGCCCGGGACCTGATTTGTGCTCAGAAGTTTAACGGACTGACAGTGCTGCCACCTCTGCTGACAGACGAGATGATCGCCCAGTATACTTC |
| | CGCCCTCCTGGCCGGTACAATTACAAGCGGCTGGACCTTCGGGGCCGGAGCTGCACTGCAGATCCCCTTTGCTATGCAGATGGCCTATAGATTCAACGGAATCGGCGTTACTCAGAACGTGC |
| | TGTACGAGAACCAGAAGCTGATCGCCAACCAGTTCAATAGCGCCATCGGAAAGATTCAGGACAGCCTGAGCTCCACAGCTAGCGCTCTGGGCAAACTGCAGGACGTTGTCAATCAGAACGCC |
| | CAGGCCCTCAACACCCTGGTGAAACAGCTGTCTAGTAACTTTGGCGCCATTAGCAGCGTGCTGAATGACATCCTCTCCCGCCTGGATCCTCCCGAGGCCGAGGTGCAGATCGACAGGCTGAT |
| | TACAGGGCGGCTGCAGTCTCTTCAGACCTACGTGACACAGCAGCTGATCAGAGCTGCCGAGATCCGGGCATCTGCCAATCTGGCCGCCACTAAGATGTCTGAGTGCGTGCTGGGACAGTCTA |
| | AACGGGTGGATTTCTGTGGCAAGGGGTACCACCTGATGTCTTTCCCCCAGTCAGCCCCCCACGGAGTGGTGTTCCTGCACGTGACATATGTCCCTGCCCAGGAAAAGAATTTCACCACCGCC |
| | CCAGCAATTTGCCACGACGGGAAAGCACACTTTCCTCGGGAGGGCGTGTTTGTGTCAAATGGGACCCACTGGTTTGTGACCCAGAGAAATTTTTACGAGCCCCAGATCATTACCACTGACAA |
| | TACATTCGTGAGCGGCAACTGCGACGTGGTGATCGGCATTGTGAATAATACAGTGTATGACCCCCTGCAGAGCGAGCTGGACAGCATCAAAGAGGAACTTGATAAAATCCATAAAAATGGCA |
| | GCGGCGGGAGCGGCGACATTATCAAGCTGCTGAACGAGCAGGTGAACAAGGAAATGCAGTCCTCCAACCTGTATATGTCCATGAGCAGCTGGTGTTACACACATTCTCTGGATGGGGCAGGC |
| | CTGTTCCTGTTTGATCACGCAGCCGAGGAGTACGAACATGCTAAGAAGCTGATTATTTTCCTGAACGAAAACAATGTGCCAGTGCAGCTGACCTCCATCTCCGCCCCCGAGCACAAGTTCGA |
| | GGGACTCACTCAGATCTTCCAGAAGGCCTATGAACACGAACAGCACATCTCCGAGTCCATTAACAATATCGTGGACCACGCAATTAAGTCTAAGGACCACGCAACCTTCAACTTTCTGCAGT |
| | GGTACGTCGCTGAGCAGCACGAAGAGGAGGTGCTGTTCAAAGACATCCTGGATAAAATCGAGCTGATCGGAAATGAGAACCATGGGCTGTACCTGGCCGACCAGTATGTGAAAGGCATTGCC |
| | AAAAGCCGCAAGAGCGGCAGCTAA (SEQ ID NO: 560) |
| Beta SpFN | ATGGATTCTAAGGGCTCCAGCCAGAAGGGAAGCAGGCTGCTGCTCCTCCTGGTGGTGTCCAACCTGCTGCTGCCACAGGGGGTGGTGGGACAGTCCAGCCAGTGTGTCAATTTCACAACTAG |
| | AACCCAGCTCCCCCCTGCCTATACTAACTCCTTCACTCGCGGGGTTTATTACCCAGACAAGGTGTTCAGGTCCTCCGTGTTGCATTCTACACAAGACTTGTTCCTGCCATTCTTTAGCAATG |
| | TGACATGGTTTCATGCCATTCACGTCAGCGGAACTAATGGCACAAAGCGCTTTGCTAACCCCGTGCTGCCCTTCAACGATGGGGTGTACTTCGCCAGCACAGAGAAAAGCAATATTATTCGC |
| | GGCTGGATCTTTGGGACTACTCTGGATTCTAAGACTCAGTCCCTGCTGATCGTGAATAACGCTACAAACGTGGTCATTAAGGTGTGCGAGTTTCAGTTCTGCAACGATCCTTTTCTGGGCGT |
| | GTATTATCACAAGAATAACAAAAGCTGGATGGAATCCGAATTCAGAGTTTACTCTTCCGCCAACAACTGCACCTTCGAGTACGTGTCCCAGCCCTTTCTGATGGACCTGGAAGGAAAGCAGG |
| | GAAACTTCAAAAATCTGCGGGAGTTCGTGTTTAAAAACATTGATGGCTACTTTAAGATCTATAGCAAACACACTCCAATCAACCTGGTGCGGGGACTCCCACAAGGATTCAGCGCTCTGGAG |
| | CCTCTGGTGGATCTGCCTATCGGGATTAACATCACTAGGTTCCAGACTCTGCACATTTCCTACCTGACACCCGGCGACTCTTCTTCTGGCTGGACCGCCGGGGCCGCCGCTTACTACGTGGG |
| | ATATCTCCAGCCACGCACCTTTCTGCTGAAGTATAACGAGAACGGCACCATTACAGATGCCGTGGACTGCGCTCTCGACCCTCTGTCCGAGACAAAGTGTACTCTGAAGTCCTTCACCGTGG |
| | AGAAAGGCATTTATCAGACTTCCAATTTCCGCGTTCAGCCCACAGAGTCCATCGTCCGCTTTCCCAACATTACTAACCTGTGCCCTTTTGGGGAGGTGTTTAATGCAACAAGATTCGCAAGC |
| | GTGTACGCTTGGAACCGGAAAAGGATTTCTAATTGTGTGGCCGATTACAGCGTGCTGTACAATAGCGCCAGCTTCAGTACTTTCAAGTGCTACGGAGTCAGCCCAACCAAACTGAACGACCT |
| | CTGCTTTACCAACGTGTACGCTGACTCCTTCGTGATCCGGGGAGACGAAGTGAGGCAGATTGCCCCCGGACAGACTGGCAATATCGCCGACTACAATTACAAACTCCCTGATGACTTCACAG |
| | GATGTGTGATCGCTTGGAACAGCAACAACCTGGACTCCAAAGTCGGAGGAAACTACAATTACCTGTACCGGCTGTTTAGAAAGTCCAACCTGAAGCCTTTTGAGAGAGATATTTCCACTGAA |
| | ATCTATCAGGCAGGAAGCACCCCATGTAATGGGGTGAAGGGATTCAACTGCTACTTCCCTCTCCAGTCCTATGGCTTTCAGCCAACCTATGGCGTCGGATACCAGCCATATAGAGTGGTGGT |
| | TCTGAGCTTCGAACTGCTCCACGCCCCAGCCACCGTGTGTGGCCCCAAGAAGTCTACCAACCTGGTTAAGAACAAATGCGTGAATTTTAACTTCAATGGACTCACTGGCACTGGAGTCCTGA |
| | CAGAGTCTAACAAGAAGTTTCTGCCATTCCAGCAGTTTGGCAGGGACATTGCCGACACCACAGATGCCGTGAGGGACCCCCAGACCCTGGAGATTCTGGATATCACTCCTTGCAGTTTTGGC |
| | GGCGTTTCCGTGATCACCCCTGGCACCAATACCTCTAACCAGGTGGCAGTGCTGTACCAGGGGGTGAACTGTACAGAGGTGCCTGTGGCCATTCATGCTGACCAGCTGACACCTACTTGGCG |
| | GGTCTATTCCACCGGCTCTAACGTGTTTCAGACTAGGGCCGGGTGTCTGATTGGGGCTGAACACGTTAACAACAGCTACGAGTGTGATATTCCAATCGGGGCAGGGATCTGCGCAAGCTACC |
| | AGACACAAACTAACTCTCCAGGCTCTGCTTCCAGCGTGGCTTCCCAGTCCATCATCGCCTACACAATGAGCCTGGGAGTGGAAAACAGCGTGGCCTACTCTAATAACAGCATCGCCATTCCA |
| | ACCAACTTCACCATCAGCGTGACTACAGAAATTCTGCCCGTGAGCATGACCAAAACTTCCGTCGACTGTACAATGTACATCTGCGGGGATTCTACAGAGTGTTCTAACCTGCTCCTGCAGTA |
| | CGGCTCCTTTTGTACGCAGCTGAATCGGGCCCTGACAGGGATCGCCGTCGAGCAGGACAAGAATACACAGGAGGTGTTCGCCCAGGTGAAGCAGATTTATAAGACTCCACCCATTAAGGACT |
| | TCGGAGGATTTAACTTCTCCCAGATTCTACCAGACCCAAGCAAGCCCTCCAAGCGGAGCTTCATCGAAGACCTGCTGTTCAACAAGGTGACCCTGGCAGATGCTGGCTTTATCAAGCAGTAC |
| | GGAGATTGCCTGGGAGATATCGCCGCTCGGGATCTCATTTGTGCTCAGAAATTCAATGGCCTGACAGTCCTGCCTCCTCTCCTGACCGATGAGATGATCGCTCAGTATACAAGCGCCCTGCT |
| | GGCTGGAACAATTACAAGCGGCTGGACATTCGGCGCCGGAGCCGCTCTGCAGATCCCATTCGCAATGCAGATGGCTTACCGCTTCAACGGGATCGGCGTGACTCAAAACGTGCTGTATGAGA |
| | ACCAAAAGCTGATCGCTAACCAGTTTAACTCCGCCATCGGGAAAATCCAGGACTCTCTCTCGTCCACAGCTTCCGCCCTGGGCAAACTGCAGGATGTGGTGAACCAGAATGCTCAGGCTCTG |
| | AACACCCTGGTGAAACAGCTGAGCAGCAATTTTGGGGCAATCAGCTCTGTGCTGAATGACATTTTGAGCAGACTGGACCCCCCCGAAGCCGAGGTGCAGATCGACAGACTGATTACAGGCAG |
| | GCTTCAGTCCCTGCAGACATATGTGACCCAGCAGCTGATTCGGGCTGCAGAGATCAGAGCCTCCGCAAACCTGGCTGCCACCAAAATGTCCGAGTGCGTGCTGGGGCAGTCCAAGAGAGTTG |
| | ACTTTTGTGGGAAGGGGTACCACCTCATGTCCTTCCCCCAGTCCGCACCACACGGAGTGGTGTTTCTGCACGTCACCTATGTGCCTGCCCAGGAGAAAAACTTCACAACCGCACCTGCTATT |
| | TGCCACGACGGCAAGGCCCATTTCCCAAGGGAAGGCGTGTTCGTGTCTAACGGAACACACTGGTTTGTCACTCAGAGAAACTTTTACGAACCACAGATTATCACCACTGACAATACCTTTGT |
| | GTCCGGCAATTGTGATGTTGTGATCGGCATCGTCAATAATACCGTGTATGATCCACTGCAGTCCGAGCTGGACAGCATCAAGGAGGAACTGGATAAAATTCACAAGAATGGCAGCGGAGGAA |
| | GCGGAGATATTATCAAGCTGCTCAATGAGCAAGTGAATAAAGAAATGCAGAGCTCTAACCTGTACATGTCCATGAGCAGCTGGTGTTATACCCACTCCCTGGACGGCGCCGGCCTGTTCCTG |
| | TTCGACCATGCCGCCGAAGAATACGAACACGCCAAGAAACTGATCATTTTCCTGAACGAGAACAACGTGCCTGTGCAGCTAACCAGCATCTCTGCCCCTGAACACAAGTTTGAGGGCCTCAC |
| | TCAGATCTTTCAGAAGGCATACGAACACGAGCAGCACATTAGCGAGAGCATCAATAACATTGTGGACCACGCTATCAAGTCCAAGGACCACGCCACCTTCAACTTCCTCCAGTGGTACGTGG |
| | CTGAGCAGCACGAGGAGGAGGTGTTGTTCAAGGATATCTTGGACAAGATCGAGCTGATTGGGAATGAGAATCACGGCCTGTATCTGGCCGACCAGTACGTGAAAGGGATCGCTAAAAGCCGG |
| | AAGTCAGGATCCTAA (SEQ ID NO: 561) |
| BQ1.1 SpFN | ATGGACTCTAAGGGCAGCAGCCAGAAAGGCAGCCGGCTGCTGCTGCTGCTGGTGGTGAGCAATCTCCTGCTGCCCCAGGGCGTGGTGGGACAGTCCTCCCAGTGCGTGAACCTGATCACCAG |
| | AACCCAGAGCTACACCAATAGCTTTACCCGGGGCGTGTACTACCCTGACAAGGTGTTCCGGAGCAGCGTGCTGCACAGCACCCAGGACCTCTTCCTGCCTTTCTTCAGCAACGTCACCTGGT |
| | TCCACGCCATCTCCGGCACCAATGGCACCAAGAGATTCGACAATCCCGTGCTGCCTTTTAACGACGGCGTGTACTTCGCCAGCACCGAGAAAAGCAATATCATCCGGGGCTGGATCTTCGGC |
| | ACCACCCTGGACAGCAAGACCCAGAGCCTGCTGATCGTGAATAACGCCACCAACGTGGTGATCAAGGTGTGTGAGTTCCAGTTCTGCAATGATCCCTTCCTGGATGTGTACTACCACAAGAA |
| | TAATAAGAGCTGGATGGAGAGCGAGTTCAGAGTGTACTCTAGCGCCAATAACTGCACCTTCGAGTACGTGTCCCAGCCTTTCCTGATGGACCTGGAAGGCAAGCAGGGCAACTTCAAGAATC |
| | TGAGAGAGTTCGTGTTCAAAAATATCGATGGCTACTTCAAGATCTACAGCAAGCACACCCCTATCAATCTGGGCCGGGACCTGCCCCAGGGCTTCTCCGCCCTGGAGCCTCTGGTGGACCTG |
| | CCCATCGGCATCAACATCACCCGGTTCCAGACCCTGCTGGCCCTGCACAGAAGCTACCTGACCCCTGGCGACAGCAGCTCTGGCTGGACAGCCGGAGCAGCTGCCTACTACGTGGGCTACCT |
| | GCAACCTCGGACCTTCCTGCTGAAGTACAACGAGAATGGCACCATCACCGACGCCGTGGATTGTGCCCTGGACCCTCTGAGCGAAACCAAGTGCACCCTGAAGTCTTTCACAGTGGAAAAGG |
| | GAATCTACCAGACAAGCAATTTCCGGGTGCAGCCCACCGAGTCCATCGTCCGGTTTCCTAACATCACCAACCTGTGCCCTTTTGACGAGGTGTTCAACGCCACCACCTTCGCCAGCGTGTAC |
| | GCCTGGAATAGAAAGCGGATCAGCAACTGCGTGGCCGACTACAGCGTGCTGTACAACTTCGCCCCTTTCTTCGCCTTCAAGTGCTACGGCGTGTCCCCCACCAAGCTGAATGACCTGTGCTT |
| | CACCAATGTGTACGCCGACAGCTTCGTGATCCGGGGCAATGAAGTGAGCCAGATCGCCCCAGGCCAGACCGGCAATATCGCCGATAATTACAAGCTGCCCGACGACTTCACAGGCTGCGTGA |
| | TCGCCTGGAATAGCAACAAGCTGGACTCTACCGTGGGGGGCAACTACAACTACCGGTACAGACTGTTCCGGAAGTCTAAGCTGAAGCCCTTCGAAAGAGACATCAGCACCGAGATCTACCAG |
| | GCCGGAAACAAGCCTTGCAATGGCGTGGCCGGCGTGAACTGCTACTTTCCCCTGCAGAGCTACGGCTTCAGGCCTACCTACGGCGTGGGCCACCAGCCTTACCCTTCCAGCAGTTCGGCCGG |
| | GACATCGCCGACACAACAGATGCAGTGAGGGACCCACAGACCCTGGAGATCCTGGACATCACCCCCTGCAGCTTCGGCGGCGTGTCCGTGATCACCCCCGGCACAAACACCAGCAATCAGGT |
| | GGCCGTGCTGTACCAGGGCGTCAATTGCACAGAGGTGCCTGTGGCCATCCACGCCGACCAGCTGACACCTACCTGGAGAGTGTACTCCACCGGCAGCAACGTGTTCCAGACCCGGGCCGGCT |
| | GCCTGATCGGCGCCGAATACGTGAACAATAGCTACGAGTGCGATATCCCCATCGGCGCCGGCATCTGCGCTAGCTACCAGACCCAGACCAAGAGCCACGGCAGCGCCAGCAGCGTGGCCTCC |
| | CAGTCCATCATCGCCTACACCATGAGCCTGGGCGCCGAGAATTCCGTGGCCTACAGCAACAATTCCATCGCTATCCCTACCAATTTCACCATCTCCGTGACAACCGAGATCCTGCCCGTGAG |
| | CATGACCAAGACCAGCGTGGACTGCACCATGTACATCTGCGGCGACTCCACCGAGTGCAGCAACCTGCTGCTGCAGTACGGCAGCTTCTGCACCCAGCTGAAGCGCGCCCTGACCGGCATTG |
| | CCGTGGAGCAGGACAAGAATACCCAGGAAGTGTTCGCCCAGGTGAAGCAGATTTACAAGACCCCCCCTATCAAGTACTTCGGCGGCTTCAATTTCAGCCAGATCCTGCCCGACCCCTCTAAA |
| | CCTAGCAAGCGGTCCTTCATCGAGGACCTGCTGTTCAATAAGGTGACACTGGCCGACGCCGGCTTCATTAAGCAGTACGGCGACTGCCTGGGCGACATCGCCGCCCGGGACCTGATCTGCGC |
| | CCAGAAGTTCAACGGCCTGACCGTGCTGCCTCCCCTGCTGACCGACGAGATGATCGCCCAGTACACCTCCGCCCTGCTGGCCGGAACCATCACCAGCGGCTGGACCTTCGGCGCCGGAGCCG |
| | CACTGCAGATCCCTTTCGCCATGCAGATGGCCTACCGGTTCAACGGCATCGGCGTGACACAGAATGTGCTGTACGAGAACCAGAAGCTGATTGCCAACCAGTTCAATAGCGCCATCGGCAAG |
| | ATCCAGGACTCCCTGAGCAGCACCGCCAGCGCCCTGGGCAAGCTGCAGGACGTGGTGAACCACAATGCCCAGGCCCTGAATACCCTGGTGAAGCAGCTGAGCAGCAAGTTCGGCGCCATCTC |
| | CAGCGTGCTGAACGATATCCTGAGCAGACTGGACCCACCTGAGGCCGAGGTGCAGATCGACAGACTGATCACCGGCAGACTGCAGTCCCTGCAGACCTACGTGACCCAGCAGCTGATCAGAG |
| | CCGCCGAGATCCGGGCCAGCGCCAATCTGGCCGCCACCAAGATGAGCGAGTGCGTGCTGGGCCAGAGCAAGAGAGTGGATTTCTGCGGCAAGGGATACCACCTGATGTCCTTCCCTCAGAGC |
| | GCCCCTCACGGCGTGGTGTTCCTGCACGTGACATACGTGCCTGCCCAGGAGAAGAATTTCACCACCGCCCCCGCCATCTGCCACGACGGAAAGGCCCACTTCCCCAGAGAGGGCGTGTTCGT |
| | GTCTAATGGCACCCACTGGTTCGTGACACAGAGAAACTTCTACGAGCCCCAGATCATCACAACCGACAACACCTTCGTGAGCGGCAATTGTGACGTGGTGATCGGCATCGTGAATAACACCG |
| | TGTACGACCCCCTGCAGAGCGAACTGGACAGCATCAAGGAGGAGCTGGACAAGATCCACAAGAACGGCTCCGGAGGCTCTGGCGATATTATCAAGCTGCTGAATGAGCAGGTGAATAAGGAG |
| | ATGCAGAGCTCCAACCTGTACATGTCCATGAGCTCCTGGTGCTACACCCACAGCCTGGACGGCGCCGGCCTGTTCCTGTTCGATCACGCCGCCGAGGAGTACGAGCACGCCAAGAAGCTGAT |
| | CATCTTCCTGAATGAGAATAATGTGCCCGTGCAGCTGACCAGCATCTCCGCCCCTGAGCACAAGTTCGAGGGCCTGACCCAGATCTTTCAGAAGGCCTACGAACACGAGCAGCACATCTCCG |
| | AGAGCATCAATAATATCGTGGACCACGCCATCAAGAGCAAGGACCACGCCACCTTCAACTTCCTGCAGTGGTACGTGGCCGAGCAGCACGAAGAGGAGGTGCTGTTCAAGGATATCCTGGAC |
| | AAGATTGAGCTGATCGGCAATGAGAACCACGGCCTGTACCTGGCCGATCAGTACGTGAAGGGCATCGCCAAGAGCAGAAAGAGCGGCAGCTAA |
| | (SEQ ID NO: 582) |
| SARS-CoV-1 | ATGGACAGCAAGGGCTCCAGCCAGAAGGGCAGCCGGCTGCTGCTGCTGCTGGTGGTGAGCAACCTGCTGCTGCCCCAGGGCGTGGTGGGCCAGAGCGACCTGGACAGATGTACAACCTTCGA |
| SpFN | CGACGTGCAGGCCCCCAATTACACCCAGCACACCAGCAGCATGCGGGGCGTGTACTACCCCGATGAGATCTTCAGAAGCGATACCCTGTACCTGACCCAGGACCTCTTCCTGCCCTTTTACA |
| | GCAATGTGACAGGCTTCCACACAATCAATCACACATTTGGCAATCCTGTGATCCCTTTTAAGGATGGCATCTACTTCGCCGCAACCGAAAAGAGCAATGTGGTGCGGGGCTGGGTGTTCGGC |
| | AGCACAATGAATAATAAGAGCCAGAGCGTGATCATCATCAATAATAGCACCAACGTGGTGATCCGGGCCTGCAATTTCGAGCTGTGCGACAATCCCTTCTTCGCCGTGAGCAAGCCAATGGG |
| | CACCCAGACCCACACAATGATCTTCGATAATGCCTTCAATTGCACCTTCGAGTACATCAGCGACGCCTTCAGCCTGGACGTGAGCGAGAAAAGCGGCAATTTCAAGCACCTGCGGGAGTTCG |
| | TGTTTAAGAATAAGGACGGCTTCCTGTACGTGTACAAGGGCTACCAGCCCATCGACGTGGTGCGGGACCTGCCCAGCGGCTTTAATACCCTGAAGCCAATCTTCAAGCTGCCTCTGGGCATC |
| | AATATCACAAACTTCCGGGCCATCCTGACCGCCTTCTCCCCCGCCCAGGACATCTGGGGCACCAGCGCCGCCGCCTACTTCGTGGGCTACCTGAAGCCCACCACATTCATGCTGAAATACGA |
| | CGAGAATGGCACCATCACCGACGCCGTGGACTGCAGCCAGAATCCCCTGGCCGAGCTGAAGTGTAGCGTCAAGAGCTTCGAGATCGACAAGGGCATCTACCAGACCAGCAACTTCAGAGTGG |
| | TGCCTAGCGGCGACGTGGTGAGATTCCCCAATATCACAAACCTGTGCCCCTTCGGCGAAGTGTTCAACGCCACAAAGTTCCCTTCTGTGTACGCCTGGGAGCGGAAGAAGATCAGCAACTGC |
| | GTGGCCGATTACAGCGTGCTGTACAATAGCACCTTCTTCAGCACCTTCAAGTGTTACGGCGTGAGCGCCACCAAACTGAATGACCTGTGCTTCAGCAACGTGTACGCCGACTCCTTCGTGGT |
| | GAAGGGCGATGACGTGAGACAGATCGCCCCTGGCCAGACCGGCGTGATCGCCGATTACAACTACAAGCTGCCCGATGATTTCATGGGCTGCGTGCTGGCCTGGAATACCAGAAATATCGACG |
| | CCACCAGCACCGGCAACTACAATTACAAGTACAGATACCTGAGACACGGCAAGCTGCGGCCCTTCGAGAGAGACATCAGCAATGTGCCTTTCAGCCCAGACGGCAAGCCTTGCACCCCTCCT |
| | GCCCTGAATTGCTACTGGCCCCTGAATGATTACGGCTTCTACACCACCACCGGCATCGGCTACCAGCCCTACCGGGTGGTGGTGCTGAGCTTCGAGCTGCTGAACGCCCCTGCCACCGTGTG |
| | CGGCCCCAAGCTGAGCACCGACCTGATCAAGAATCAGTGCGTGAATTTTAACTTCAACGGCCTGACCGGCACCGGCGTGCTGACCCCCAGCTCTAAGCGGTTCCAGCCCTTCCAGCAGTTCG |
| | GCAGAGACGTGAGCGACTTCACCGACTCTGTGCGGGATCCCAAGACCTCCGAGATCCTGGACATCAGCCCTTGCGCCTTCGGCGGCGTGAGCGTGATCACACCCGGCACCAATGCCAGCAGC |
| | GAAGTGGCCGTGCTGTACCAGGACGTGAATTGCACCGACGTGTCCACCGCCATCCACGCCGACCAGCTGACACCCGCCTGGAGAATCTACAGCACCGGCAATAACGTGTTCCAGACCCAGGC |
| | CGGCTGCCTGATCGGAGCAGAGCACGTGGACACCAGCTACGAATGCGACATCCCTATCGGAGCCGGCATCTGCGCCTCCTACCACACCGTGAGCCTGCTGCGGAGCACCAGCCAGAAGTCCA |
| | TCGTGGCCTACACCATGAGCCTGGGCGCCGACAGCTCCATTGCCTACTCCAACAATACCATCGCCATCCCTACCAACTTCAGCATCAGCATCACCACCGAAGTGATGCCTGTGAGCATGGCC |
| | AAGACAAGCGTGGACTGCAATATGTACATCTGCGGCGACTCCACCGAGTGCGCCAACCTGCTGCTGCAGTACGGCTCCTTCTGCACCCAGCTGAATAGAGCCCTGAGCGGCATCGCCGCCGA |
| | GCAGGACAGAAATACCCGGGAGGTGTTCGCCCAGGTGAAGCAGATGTACAAGACCCCCACACTGAAGTACTTCGGCGGCTTCAATTTCAGCCAGATCCTGCCCGACCCTCTGAAGCCAACCA |
| | AGCGGAGCTTCATCGAAGACCTGCTGTTCAACAAGGTGACCCTGGCCGACGCCGGCTTCATGAAGCAGTACGGCGAGTGCCTGGGCGATATCAACGCCCGGGACCTGATCTGCGCCCAGAAG |
| | TTCAACGGCCTGACCGTGCTGCCCCCTCTGCTGACCGACGACATGATCGCCGCCTACACAGCCGCCCTGGTGAGCGGAACCGCTACAGCCGGCTGGACATTCGGCGCCGGCGCCGCCCTGCA |
| | GATCCCATTCGCCATGCAGATGGCCTACAGATTCAATGGCATCGGGGTGACACAGAATGTGCTGTACGAGAATCAGAAGCAGATCGCCAACCAGTTCAATAAGGCCATCTCCCAGATCCAGG |
| | AGTCCCTGACCACCACCTCCACCGCCCTGGGCAAGCTGCAGGATGTGGTGAATCAGAATGCCCAGGCCCTGAATACCCTGGTGAAGCAGCTGAGCTCCAACTTCGGCGCCATCAGCTCCGTG |
| | CTGAACGACATCCTGAGCCGGCTGGACCCTCCTGAGGCCGAAGTGCAGATCGACAGACTGATCACAGGCCGGCTGCAGAGCCTGCAGACATACGTGACCCAGCAGCTGATCAGAGCCGCCGA |
| | AATCAGAGCCAGCGCCAATCTGGCCGCCACCAAGATGAGCGAGTGCGTGCTGGGCCAGAGCAAGAGAGTGGACTTCTGCGGCAAGGGCTACCACCTGATGAGCTTCCCTCAGGCCGCCCCTC |
| | ACGGCGTGGTGTTCCTGCACGTGACCTACGTGCCCAGCCAGGAGCGGAACTTCACCACCGCCCCTGCCATCTGCCACGAGGGAAAGGCCTACTTCCCTCGGGAGGGCGTGTTCGTGTTTAAT |
| | GGCACAAGCTGGTTCATCACCCAGAGAAACTTCTTCAGCCCCCAGATCATCACAACCGACAACACCTTCGTGTCCGGCAACTGCGATGTGGTGATCGGAATCATCAATAATACCGTGTACGA |
| | CCCCCTGCAGAGCGAGCTGGACTCCATCAAGGAGGAGCTGGACAAGATCCACAAGAATGGGAGCGGCGGCTCCGGCGATATCATCAAGCTGCTGAACGAGCAGGTGAATAAGGAGATGCAGA |
| | GCAGCAACCTGTACATGAGCATGTCTAGCTGGTGTTACACCCACAGCCTGGATGGCGCCGGCCTGTTCCTGTTCGACCACGCCGCCGAGGAGTACGAGCACGCCAAGAAGCTGATCATCTTC |
| | CTGAATGAGAATAATGTGCCCGTGCAGCTGACCTCTATCAGCGCCCCCGAGCACAAGTTCGAAGGCCTGACACAGATCTTCCAGAAGGCCTACGAGCACGAGCAGCACATCTCCGAGAGCAT |
| | CAACAATATCGTGGACCACGCCATCAAGAGCAAGGACCACGCCACCTTCAATTTCCTGCAGTGGTACGTGGCCGAGCAGCACGAGGAGGAGGTGCTGTTCAAAGACATCCTGGACAAGATCG |
| | AGCTGATCGGCAACGAGAATCACGGCCTGTACCTGGCCGATCAGTACGTGAAGGGCATCGCCAAGAGCAGAAAGTCCGGCAGCTAA (SEQ ID NO: 563) |
| Khosta-2 | ATGGACAGCAAGGGAAGCTCCCAGAAAGGGTCCAGGCTGCTGCTGCTGCTCGTCGTGAGCAACCTGCTGCTGCCTCAGGGCGTGGTGGGACAATCTAGCCAGGATGGGTGCGCTACACTGAG |
| SpFN | CACCAAGCTGACTCCCACCCAGCTGCAGGTGAACTCTACTCGGAGGGGATTTTACTATTTCGACGATATTTATCGCAGCAGCCTGAGGGTGCTGGTGCCAGGCTATTTCCTGCCATTCGGAA |
| | CAAACATCACTAGATACCAGGCCCAGGTGATCAACTGGACCGCCATTTTCTTTGACAACCCTGTCATCCCTTTTAATGACGGAGTGTACTTCGCCGCACTGGAGCACTCCAATGTGGTGAGG |
| | GGGTGGATCTTTGGAACAACACTGGATAACACTACCCAGTCCGCAATCGTCGTGAACAACGCCACCCACATCCTGATTAGTGTGTGTTACTTCAACGTGTGCAAGGAACCTATGTTCGCCGT |
| | GTCCAATTTCAAGCCCTACAAGAGCTGGGTGTACGATAGGGCCAACAACTGCACCTTTAATCGGGCATACCCCCTCAACATCAGCACAACTCCCGAGCCTGGGAAGTTCAAGGCACTGAGAG |
| | AGCAGGTGTTTAGGTACCAGGACGGCTTTCTGTACATCTATCACAGCTACGAGTCCATTAATTCCGACACTCTGCCCCCTGGATTCTCCGTGCTGAAACCTATGCTGAAGCTGCCTCTGGGA |
| | CTGAATATCACAAGATTTCGGGTGGTGATGGCCATGCACTCTCTGACTACAAGCAACTTCAACACCCACTCTGTGAATTATTTCGTGGGGCACCTGAAACCACTGACAATGCTGGTGGAATT |
| | CTCCCCTAACGGCACAATTATTGACGCAATCGATTGTTCCCAGGATCCCCTGAGCGAGCTGAAATGCACCACAAAGTCATTCTCCGTGGAGAAGGGCATCTATCAGACTTCCAATTTCCGGG |
| | TGAGCCCCTCCCTGGACGTGGTGCGCTTCCCAAATATGACCAACATCTGCCCCTTTGACCAGGTGTTTAATAAGACTCAGTTCCCTTCTGTGTACGCCTGGGAAAGAGTGAGGATCAGCGAC |
| | TGCGTGAGCGACTACACAGTGCTGTATAATTCTTCCGCTTCTTTTAGCACATTCAAATGCTACGGCGTGTCCCCAACAAAGCTGAACGACCTGTGCTTCTCCGGAGTGTATGCCGATTACTT |
| | CGTGGTGAAGGGGGACCACGTCCACCAGATTGCACCTGGACAGACAGGCGTGATCGCCGACTATAATTACAAGCTCCCTTCCGAATTTGTGGGCTGCATTCTGGCTTGGAACACAAGGACTA |
| | TTGACTCCAAGAGGGGGTTTTACTACCGGCTGTTCAGACACGGCAATATCCGGCCATATGAGAGGGATACCTCCAACGTGCCCTACAATGCTGCCGGGGGAACCTGCAACCAGCCAGGAACT |
| | CACAACTGCTACGAGCCTCTGCAGGATTACGGCTTCACTTCCACTAGCGGAGTGGGGTACCAGCCCTTTCGCGTGGTGGTGCTGTCTTTCGAACTGCTGAACGCCCCTGCCACAGTCTGTGG |
| | GCCTAAGCAGAGCACTGACCTGGTGAAGAATAAGTGCGTGAATTTCAACTTCAATGGGCTGACCGGGACTGGGGTGCTGACTGACTCCAATAAGAAGTTTCAGCCATTTCAGCAGTTCGGCA |
| | GAGATAGCGCTGATTTCACCGACTCTGTGAAAGATCCAAAGACCCTGGAGATCCTGGATATCACACCCTGTAGCTATGGCGGCGTGTCTGTGATTACCCCTGGCACCAATACATCTGACAGC |
| | GTGGCCGTCCTGTATCAGGACGTGAACTGTACCGATGTGCCCACAATGCTGCATATGGACCAGGTGTCCAACGATTGGAGGGTGTATGCTGTGAACACCGACGGAAACATGTTCCAGACACA |
| | GGCTGGGTGCCTCGTGGGAGCTACCTATGACAATACATCCTATGAGTGCGATATCCCCGTGGGAGCTGGCGTGTGTGCCAAATTCCAGACCACCGGCAGCGCCTCTCAGAGCTCCATTCTGG |
| | CATATACAATGAGCCTGGGAGAGGACTCCAATGTGGCCTACTCCAACAATAGCATTGCAATCCCTACCAATTTTACAATCAGCGTGACAACTGAAGTCCTGCCAGTCTCCATGACCAAGACA |
| | GCCGTGGATTGCAACATGTATATCTGCGGAGATTCTACTGAGTGTTCCAGCCTGCTGCTGCAGTACGGCAGCTTCTGCCAGCAGCTGAACAGGGCTCTGGCCGGCGTGTCTGTGGAGCAGGA |
| | CAAGAATACACAGGACGTGTTTGCTCAGGTGAAGTCTATCTACAAGGTGTCTGCTATTAAGGACTTTGGCGGCTTTAATTTTAGCCAGATTCTGCCTGACCCAGCCAAGCCTAGCAAACGCT |
| | CTTTTATCGAAGACCTGCTGTACAATAAGGTGACTCTGGCTGACCCAGGCTTTATGAAGCAGTACGGGGACTGTCTGGGAGGCGTGAACGCCAGGGACCTGATTTGTGCACAGAAGTTCAAC |
| | GGCCTGACCGTGCTGCCCCCCCTGCTGACAGACGATATGATTGCAGCTTACACATCCGCCTTAGTGAGCGGCACCGCCCTGAGCGGATTCACATTCGGGGCTGGAGCCGCACTGCAGATCCC |
| | CTTTGCCATGCAGATGGCCTACAGGTTCAACGGCATCGGCGTGACTCAGAACGTTCTGTATGAGAATCAGAAGCAGATTGCAAACCAGTTTAATAAGGCAATCTCCCAGATTCAGGAGAGCC |
| | TGACAACCACCGCCACCGCCCTGGGCAAGCTGCAGGATGTGATCAACCAGAACGCTATCGCCCTCAACACCCTGGTGAAACAGCTGAGCTCCAACTTCGGAGCTATCTCCTCCGTCCTGAAC |
| | GACATTCTGTCCAGACTGGATCCACCTGAAGCCGAGGTGCAGATTGACAGACTCATTACAGGCAGACTGCAGTCACTGCAGACCTACGTGACCCAACAACTGATCAGAGCCGCAGACATCAG |
| | GGCAAGCGCCAACCTGGCCGCCACCAAGATGAGCGAATGCGTGCTGGGACAGTCTAAGCGGGTGGACTTCTGTGGCAAAGGCTATCACCTGATGTCCTTCCCACAGGCCGCACCTCACGGAG |
| | TGGTGTTCCTGCACGTCACTTATGTGCCAAGCCAGGATAAGAATTTTACAACAGCACCTGCTATCTGCCACAAGGGCAAGGCCTACTTCCCCAGAGAGGGAGTGTTCGTGACAAACGGAACA |
| | CACTGGTTCGTGACCCAGCGCAACTTTTATCAGCCTGAGGTGATCACAACAGAGAACACATTCGAGAGTGGCAATTGCGACGTCGTGATCGGGATCGTGAACAACACCGTCTACGACCCTCT |
| | GCAGTCCGAGCTGGATTCTATTAAGGAGGAGCTGGATAAGATCCACAAGAACGGATCCGGCGGAAGCGGCGATATTATCAAACTGCTGAATGAGCAGGTGAACAAGGAGATGCAATCCTCCA |
| | ACCTCTACATGTCTATGAGCAGCTGGTGTTATACACATTCCCTGGACGGAGCCGGCCTGTTCCTGTTCGACCACGCCGCCGAGGAATACGAGCACGCCAAGAAGCTGATCATCTTCCTGAAT |
| | GAAAACAATGTGCCCGTGCAGCTGACTAGCATCTCAGCCCCTGAGCATAAATTCGAGGGACTGACCCAGATTTTCCAGAAGGCCTACGAGCATGAGCAGCACATTTCCGAATCCATCAATAA |
| | CATTGTGGACCACGCCATTAAGTCCAAGGACCACGCAACCTTTAACTTTCTGCAGTGGTATGTGGCCGAGCAGCACGAGGAGGAGGTGCTGTTTAAAGATATCCTGGATAAAATTGAGCTGA |
| | TCGGCAACGAGAATCACGGCCTGTATCTGGCTGACCAGTACGTGAAGGGGATCGCAAAGAGCCGCAAAAGTGGGAGCTAA (SEQ ID NO: 564) |
| MERS SpFN | ATGGACAGCAAGGGCAGCAGCCAGAAAGGCAGCCGGCTGCTGCTGCTGCTGGTGGTGTCCAACCTGCTGCTGCCCCAGGGGGTGGTTGGACAGTATGTGGACGTTGGACCAGACTCAGTGAA |
| | GTCTGCCTGTATTGAAGTGGACATCCAGCAGACCTTCTTCGACAAGACCTGGCCACGCCCTATTGACGTGTCTAAGGCCGACGGGATCATCTACCCACAGGGGCGGACATATTCCAATATCA |
| | CAATTACTTACCAGGGACTGTTTCCCTACCAGGGCGACCACGGCGACATGTACGTGTATAGCGCAGGCCATGCCACCGGCACCACTCCCCAGAAACTCTTCGTGGCAAACTATTCCCAGGAT |
| | GTGAAGCAGTTCGCTAATGGGTTTGTGGTGAGGATCGGAGCCGCCGCTAATAGCACAGGCACTGTGATCATTAGCCCTTCCACATCCGCTACCATTAGAAAGATTTATCCCGCCTTCATGCT |
| | GGGCTCCAGCGTGGGAAATTTCAGTGACGGCAAGATGGGGAGATTCTTCAACCACACACTGGTGCTCCTGCCTGATGGCTGCGGCACCCTGCTGCGGGCCTTCTACTGCATCCTGGAGCCTA |
| | GGAGCGGAAACCACTGTCCTGCAGGCAATAGCTACACCTCTTTTGCCACCTATCACACCCCTGCAACAGACTGTTCCGACGGAAACTACAACAGAAACGCTAGCCTGAATAGCTTTAAGGAG |
| | TACTTTAATCTGAGAAACTGCACTTTCATGTACACATACAATATTACCGAGGACGAAATCCTGGAGTGGTTCGGCATTACCCAGACAGCACAGGGAGTGCACCTCTTTTCTTCTAGGTACGT |
| | GGACCTGTATGGAGGAAACATGTTTCAGTTCGCTACCCTGCCAGTCTACGACACAATTAAGTACTATTCCATCATCCCCCACAGCATTAGATCTATCCAGAGCGACCGGAAGGCCTGGGCCG |
| | CCTTTTATGTGTACAAGCTGCAGCCCCTGACTTTTCTGCTGGACTTCAGCGTGGATGGCTACATTAGACGGGCTATCGATTGCGGATTTAATGACCTGAGCCAGCTGCACTGTTCCTACGAG |
| | TCCTTTGATGTCGAGTCCGGCGTGTACAGCGTGAGCAGCTTCGAAGCCAAACCCAGCGGGTCCGTGGTGGAACAGGCCGAAGGCGTCGAGTGCGACTTTTCCCCCCTGCTGAGCGGAACACC |
| | ACCCCAGGTGTACAACTTTAAGCGGCTCGTGTTCACTAACTGCAATTACAATCTGACCAAACTGCTGAGCCTGTTCAGCGTGAACGATTTTACATGTTCTCAGATCTCTCCCGCAGCCATCG |
| | CCTCTAACTGCTATTCCAGCCTGATTCTCGACTACTTCTCTTATCCCCTGAGCATGAAGTCTGATCTGAGCGTGAGCAGCGCCGGGCCCATTTCCCAGTTCAACTACAAGCAGTCTTTCAGC |
| | AATCCAACATGTCTGATTCTGGCCACCGTGCCTCACAACCTGACAACCATTACCAAACCCCTGAAGTACTCTTACATCAATAAATGTAGCAGGTTCCTGAGCGATGATAGGACCGAGGTGCC |
| | CCAGCTGGTGAATGCAAACCAGTACTCTCCTTGTGTGAGCATCGTGCCCTCCACTGTGTGGGAGGACGGAGACTACTACCGCAAGCAGCTGTCCCCCCTGGAAGGAGGAGGCTGGCTGGTGG |
| | CCTCCGGCAGCACAGTCGCTATGACCGAACAGCTGCAGATGGGATTTGGCATCACTGTGCAGTATGGAACAGATACTAACAGTGTGTGTCCTAAACTGGAGTTCGCCAACGACACTAAAATC |
| | GCAAGCCAGCTGGGAAATTGCGTGGAGTATAGCCTGTATGGCGTGAGCGGCCGGGGAGTGTTCCAGAACTGCACTGCCGTGGGCGTGAGACAGCAGAGGTTCGTGTACGATGCTTACCAGAA |
| | CCTGGTTGGCTACTATAGCGACGATGGGAACTACTATTGTCTGAGAGCCTGTGTGTCTGTGCCTGTGAGCGTGATCTACGACAAGGAGACAAAAACACATGCCACCCTGTTCGGGTCCGTGG |
| | ATCTCCACAGGCAGCCGCTCCGCCCGCAGCGCAATCGAAGACCTGCTGTTCGATAAGGTGACCATCGCTGATCCAGGCTATATGCAGGGGTATGACGACTGCATGCAGCAGGGCCCCGCCTC |
| | TCTTGTGAACACATCTCCAGCACCATGAGCCAGTACTCCCGGTCCACACGCTCCATGCTGAAACGCAGGGACTCCACATACGGCCCACTGCAGACCCCTGTGGGCTGCGTGCTGGGGCTGGT |
| | GAACAGCAGCCTGTTCGTGGAAGACTGTAAACTGCCACTGGGCCAATCCCTGTGCGCTCTGCCTGACACACCTAGCACACTGACACCAGCCTCTGTCGGCTCCGTGCCCGGAGAGATGCGGC |
| | TGGCCTCTATTGCCTTTAATCATCCAATCCAGGTGGATCAGCTGAACAGCTCATACTTCAAGCTGAGCATCCCCACAAATTTCTCCTTCGGCGTGACACAGGAATATATCCAGACAACAATT |
| | CAGAAAGTGACTGTGGACTGCAAGCAGTATGTGTGTAACGGCTTCCAGAAGTGCGAGCAGCTGCTGCGGGAGTATGGCCAGTTCTGCTCTAAGATCAATCAGGCACTGCATGGGGCAAACCT |
| | GCGCCAGGACGACAGCGTGAGAAACCTCTTCGCTTCTGTGAAAAGCAGCCAGAGCTCTCCCATCATCCCAGGCTTCGGGGGCGACTTTAACCTGACACTCCTGGAGCCAGTGAGCGCAAGGG |
| | ACCTGATTTGCGCACAGTACGTGGCCGGATACAAGGTTCTGCCTCCACTGATGGATGTGAACATGGAAGCCGCCTATACCTCCTCCCTGCTGGGCAGCATCGCCGGGGTTGGCTGGACAGCC |
| | GGCCTGAGCTCTTTCGCTGCAATCCCATTCGCCCAGTCTATCTTCTATAGACTGAACGGCGTGGGGATCACCCAGCAGGTGCTGTCCGAAAATCAGAAACTGATCGCTAATAAGTTCAACCA |
| | GGCCCTGGGCGCTATGCAGACCGGATTCACCACAACTAACGAAGCCTTCCACAAGGTGCAGGATGCCGTGAATAACAACGCCCAGGCACTGTCCAAGCTGGCCTCCGAGCTGTCTAATACAT |
| | TTGGGGCCATCTCAGCCAGCATCGGGGACATCATCCAGAGACTGGACCCACCTGAGCAGGATGCTCAGATCGACAGGCTGATCAACGGCAGGCTGACAACACTGAACGCTTTCGTGGCCCAG |
| | CAGCTGGTGAGAAGCGAGTCCGCCGCCCTGTCAGCTCAGCTGGCCAAAGATAAGGTCAACGAATGCGTGAAGGCCCAGTCTAAGCGGTCTGGATTCTGCGGCCAGGGCACTCACATTGTCTC |
| | TTTCGTGGTGAACGCCCCAAACGGCCTGTACTTTATGCACGTGGGCTACTATCCATCTAACCATATCGAGGTGGTGAGCGCTTACGGCCTCTGTGACGCCGCCAACCCTACAAATTGTATCG |
| | CTCCTGTGAACGGCTACTTTATCAAGACCAATAATACACGCATTGTGGACGAATGGAGCTACACTGGCAGCTCCTTCTACGCCCCCGAGCCAATCACTAGCCTGAACACTAAGTACGTGGCC |
| | CAAGAAGCTGATCATCTTTCTGAACGAGAACAATGTGCCAGTGCAGCTGACCAGCATCTCCGCCCCCGAGCACAAGTTCGAGGGACTGACCCAGATTTTCCAGAAGGCATACGAGCACGAGC |
| | ACCACAGGTCACTTACCAGAATATCAGCACTAACCTGCCCCCTCCTCTGCTGGGGAACTCCACCGGCATCGACTTCCAGGACGAGCTCGACGAGATTCATAAGCAGGGCTCCGGCGGCAGCG |
| | GCGACATCATCAAGCTGCTCAATGAGCAGGTGAACAAGGAAATGCAGTCTAGCAACCTGTATATGTCCATGTCCTCTTGGTGTTACACACACAGCCTGGACGGCGCCGGACTGTTCCTGTTC |
| | GATCACGCCGCCGAGGAATATGAGCACGCGCATATTTCCGAAAGCATTAATAACATCGTGGATCACGCCATCAAGAGCAAGGACCACGCAACATTCAACTTCCTGCAGTGGTACGTGGCCGA |
| | GCAGCACGAGGAGGAGGTGCTGTTTAAAGACATTCTGGACAAAATTGAGCTGATTGGAAATGAGAATCATGGCCTGTACCTGGCCGACCAGTATGTGAAGGGGATCGCAAAGAGCCGCAAAT |
| | CCGGCAGCTAA (SEQ ID NO: 565) |
| BQ1.1_S2P | ATGGATTCCAAGGGCAGCAGCCAGAAGGGCAGCCGGCTGCTGCTGCTGCTGGTGGTGAGCAACCTGCTGCTGCCTCAGGGGGTGGTGGGACAGTCCAGCCAGTGCGTGAACCTGATCACCAG |
| | AACCCAGAGCTACACAAATTCTTTCACCCGGGGGGTGTACTACCCCGATAAAGTGTTCAGAAGCTCCGTGCTGCACAGCACCCAGGACCTCTTCCTGCCCTTTTTCAGCAATGTGACCTGGT |
| | TCCATGCTATCAGCGGCACCAACGGCACCAAGAGATTCGACAACCCTGTGCTGCCCTTCAATGACGGCGTGTACTTCGCCAGCACCGAGAAAAGCAATATCATCAGAGGCTGGATCTTCGGC |
| | ACCACACTGGACAGCAAGACCCAGAGCCTGCTGATCGTGAACAACGCAACCAACGTGGTGATCAAGGTGTGCGAGTTCCAGTTCTGCAACGACCCCTTCCTGGACGTGTACTACCACAAGAA |
| | CAATAAGAGCTGGATGGAGAGCGAATTCCGGGTGTACTCCAGCGCCAATAATTGCACCTTCGAGTACGTGAGCCAGCCCTTCCTGATGGACCTGGAGGGCAAGCAGGGCAACTTCAAGAATC |
| | TGCGGGAATTCGTGTTCAAGAACATCGACGGCTACTTCAAGATCTACAGCAAGCACACCCCTATCAACCTGGGCCGGGACCTGCCCCAGGGCTTCTCCGCCCTGGAGCCTCTGGTGGACCTG |
| | CCCATCGGCATCAATATTACCCGGTTCCAGACACTGCTGGCCCTGCACAGGAGCTACCTGACCCCAGGCGACAGCAGCAGCGGATGGACAGCCGGCGCCGCTGCCTACTACGTGGGCTACCT |
| | GCAGCCCCGGACCTTCCTGCTGAAGTACAATGAGAACGGCACCATCACAGACGCCGTGGACTGCGCCCTGGACCCACTGAGCGAAACCAAGTGCACACTGAAGTCCTTCACAGTGGAGAAGG |
| | GCATCTACCAGACAAGCAATTTCCGGGTGCAGCCAACCGAGAGCATCGTGAGATTCCCTAATATCACCAACCTGTGCCCTTTTGACGAAGTGTTCAATGCCACCACCTTCGCCAGCGTGTAC |
| | GCCTGGAATCGGAAGAGAATCAGCAACTGCGTGGCCGACTACAGCGTGCTGTACAATTTTGCCCCTTTCTTCGCCTTCAAGTGCTACGGCGTGAGCCCCACAAAGCTGAATGACCTGTGCTT |
| | CACCAATGTGTACGCCGACAGCTTCGTGATCCGGGGCAACGAGGTGAGCCAGATCGCCCCTGGCCAGACAGGCAACATCGCAGATTACAATTACAAGCTGCCCGACGACTTCACCGGCTGTG |
| | TGATCGCCTGGAATTCTAATAAGCTGGACAGCACCGTGGGCGGCAACTACAACTACCGGTACAGACTGTTCCGCAAGAGCAAGCTGAAGCCCTTCGAGCGGGACATCAGCACCGAGATCTAC |
| | CAGGCCGGCAACAAGCCCTGCAATGGCGTGGCCGGCGTGAACTGCTACTTCCCCCTGCAGAGCTACGGCTTCAGACCCACCTACGGCGTGGGCCACCAGCCTTACAGAGTGGTGGTGCTGAG |
| | TCCCCATCGGCGCCGGCATCTGCGCCAGCTACCAGACCCAGACAAAGAGCCACGGCAGCGCCAGCTCCGTGGCCAGCCAGTCTATCATCGCCTACACCATGAGCCTGGGCGCCGAGAATAGC |
| | GCTTCGAGCTGCTGCATGCCCCCGCCACCGTGTGCGGCCCCAAAAAGAGCACCAACCTGGTGAAGAATAAGTGTGTGAACTTCAATTTCAATGGCCTGACCGGCACCGGCGTGCTGACCGAG |
| | AGCAATAAGAAGTTCCTGCCCTTCCAGCAGTTCGGCCGGGATATCGCCGACACAACCGACGCCGTGCGGGATCCTCAGACCCTGGAGATTCTGGACATCACCCCTTGCTCCTTCGGCGGCGT |
| | GAGCGTGATCACCCCCGGCACAAATACCTCCAATCAGGTGGCCGTGCTGTACCAGGGCGTGAATTGCACAGAAGTGCCCGTGGCCATCCACGCCGACCAGCTGACCCCCACCTGGAGAGTGT |
| | ACAGCACAGGGAGCAACGTGTTCCAGACCCGGGCCGGCTGCCTGATTGGCGCCGAGTACGTGAATAACAGCTACGAGTGCGACATGGCCTACTCCAACAATAGCATCGCCATCCCCACCAAT |
| | TTCACCATCAGCGTGACCACCGAGATTCTGCCCGTGAGCATGACAAAGACAAGCGTGGACTGTACCATGTACATCTGCGGCGACAGCACCGAGTGCAGCAACCTGCTGCTGCAGTACGGCAG |
| | CTTCTGCACCCAGCTGAAGCGCGCCCTGACCGGCATCGCCGTGGAGCAGGACAAGAACACCCAGGAGGTGTTCGCCCAGGTGAAGCAGATCTACAAGACCCCCCCCATCAAGTACTTCGGCG |
| | GCTTCAATTTCTCTCAGATCCTGCCCGACCCCTCCAAGCCTAGCAAGCGGTCCTTCATCGAGGACCTGCTGTTCAATAAGGTGACACTGGCCGATGCCGGCTTCATCAAGCAGTACGGCGAC |
| | TGCCTGGGCGATATCGCCGCCCGGGACCTGATCTGCGCCCAGAAGTTCAACGGCCTGACAGTGCTGCCCCCCCTGCTGACCGACGAGATGATCGCCCAGTACACCAGCGCCCTCCTGGCCGG |
| | CACCATCACCTCTGGCTGGACATTCGGCGCTGGCGCCGCCCTGCAGATCCCTTTCGCCATGCAGATGGCCTACCGGTTCAATGGGATCGGAGTGACCCAGAATGTGCTGTACGAAAATCAGA |
| | AGCTGATCGCCAACCAGTTCAATAGCGCCATCGGCAAGATCCAGGACAGCCTGAGCAGCACCGCCTCCGCCCTGGGCAAGCTGCAGGACGTGGTGAACCACAATGCCCAGGCCCTGAACACC |
| | CTGGTGAAGCAGCTGAGCAGCAAGTTCGGCGCCATCAGCAGCGTGCTGAATGACATCCTGAGCCGGCTGGACCCTCCTGAGGCCGAGGTGCAGATCGACCGGCTGATCACCGGCAGACTGCA |
| | GAGCCTGCAGACATACGTGACCCAGCAGCTGATCCGGGCCGCCGAGATCCGGGCCTCCGCCAATCTGGCCGCCACCAAGATGTCTGAGTGCGTGCTGGGCCAGAGCAAGCGGGTGGACTTCT |
| | GCGGGAAGGGCTACCACCTGATGAGCTTCCCTCAGTCCGCCCCTCACGGCGTGGTGTTCCTGCACGTGACATACGTGCCCGCCCAGGAGAAGAATTTCACCACCGCCCCAGCCATCTGCCAC |
| | GACGGCAAGGCCCACTTTCCCAGAGAAGGCGTGTTCGTGAGCAACGGCACCCACTGGTTCGTGACCCAGAGAAATTTCTACGAGCCCCAGATCATCACCACCGACAATACCTTCGTGAGCGG |
| | CAACTGTGATGTGGTGATCGGCATCGTGAATAACACCGTGTACGACCCTCTGCAGCCTGAGCTGGACTCCTTCAAGGAGGAGCTGGACAAGTACTTCAAGAACCACACCAGCCCTGATGTGG |
| | ACCTGGGCGATATCTCCGGCATCAACGCCAGCGTGGTGAATATTCAGAAGGAGATCGACAGACTGAACGAGGTGGCCAAGAACCTGAACGAGAGCCTGATCGACCTGCAGGAACTGGGCAAG |
| | TACGAGCAGTACATCAAGTGGCCATGGTACATCTGGCTGGGCTTCATCGCCGGCCTGATCGCCATCGTGATGGTGACAATCATGCTGTGCTGCATGACCTCCTGCTGCTCCTGCCTGAAGGG |
| | CTGTTGCAGCTGCGGCAGCTGCTGCAAGTTCGACGAGGACGATTCCGAGCCCGTGCTGAAGGGCGTGAAGCTGCACTACTAA (SEQ ID NO: 569) |
| SARS_S2P | ATGGATTCCAAGGGCAGCTCCCAGAAGGGCTCCCGGCTGCTGCTGCTGCTGGTGGTGTCCAACCTGCTGCTGCCTCAGGGCGTGGTGGGCCAGAGCGACCTGGACAGATGCACAACCTTTGA |
| | TGACGTGCAGGCCCCCAACTACACACAGCACACCAGCAGCATGCGGGGCGTGTACTACCCTGACGAGATCTTCCGGAGCGACACCCTGTACCTGACACAGGACCTCTTCCTGCCCTTCTACA |
| | GCAACGTGACCGGCTTCCACACCATCAACCACACCTTCGGCAATCCTGTGATCCCCTTCAAGGACGGCATCTACTTCGCTGCCACCGAGAAGTCCAATGTGGTGCGGGGCTGGGTGTTCGGC |
| | AGCACCATGAATAATAAGAGCCAGAGCGTGATCATCATCAATAATAGCACCAATGTGGTGATCAGGGCCTGCAATTTCGAGCTGTGCGACAATCCCTTCTTCGCCGTGAGCAAGCCTATGGG |
| | CACCCAGACCCACACCATGATCTTCGACAATGCCTTCAATTGCACCTTCGAGTACATCAGCGACGCCTTTAGCCTGGACGTGTCCGAAAAAAGCGGCAATTTCAAGCACCTGCGGGAGTTCG |
| | TGTTCAAGAATAAGGACGGCTTTCTGTACGTGTACAAGGGCTACCAGCCCATCGACGTGGTGCGCGACCTGCCCAGCGGCTTCAATACCCTGAAGCCCATCTTCAAGCTGCCCCTGGGCATC |
| | AATATCACCAACTTTCGGGCCATCCTGACCGCCTTCAGCCCTGCCCAGGATATCTGGGGCACCAGCGCCGCCGCCTACTTCGTGGGCTACCTGAAGCCCACAACATTCATGCTGAAGTACGA |
| | CGAGAATGGCACCATCACCGACGCCGTGGACTGCAGCCAGAATCCTCTGGCCGAGCTGAAGTGCAGCGTGAAGTCCTTTGAGATCGATAAGGGAATCTACCAGACCAGCAACTTCCGGGTGG |
| | TGCCTAGCGGCGACGTGGTGCGGTTCCCTAATATCACCAACCTGTGCCCCTTCGGCGAGGTGTTCAATGCCACCAAGTTCCCCTCCGTGTACGCCTGGGAGAGAAAGAAGATCAGCAATTGC |
| | GTGGCCGACTACAGCGTGCTGTACAATTCTACCTTCTTCTCCACCTTCAAGTGCTACGGCGTGTCTGCCACCAAGCTGAACGACCTGTGCTTTTCCAACGTGTACGCCGACAGCTTCGTGGT |
| | GAAGGGCGACGACGTGCGGCAGATCGCCCCTGGCCAGACCGGCGTGATCGCTGATTACAACTACAAGCTGCCAGATGACTTCATGGGCTGCGTGCTGGCCTGGAACACCAGAAATATCGACG |
| | CCACCAGCACCGGAAACTACAACTACAAGTACCGGTACCTGCGGCACGGCAAGCTGCGGCCCTTTGAGCGGGACATCAGCAACGTGCCTTTCAGCCCTGACGGAAAGCCCTGTACCCCCCCC |
| | GCCCTGAACTGCTACTGGCCTCTGAATGACTACGGCTTCTACACCACAACCGGCATCGGCTACCAGCCTTACAGAGTGGTGGTGCTGAGCTTCGAGCTGCTGAATGCCCCTGCCACCGTGTG |
| | CGGCCCAAAGCTGAGCACCGACCTGATCAAGAACCAGTGCGTGAACTTCAATTTCAACGGCCTGACCGGCACCGGCGTGCTGACCCCCAGCAGCAAGAGATTCCAGCCTTTCCAGCAGTTCG |
| | GCAGAGACGTGAGCGACTTCACAGACAGCGTGCGGGACCCCAAGACATCTGAGATCCTGGATATCAGCCCCTGCGCCTTCGGCGGAGTGTCCGTGATTACCCCTGGCACCAACGCCAGCAGC |
| | GAGGTGGCCGTGCTGTACCAGGACGTGAATTGTACCGACGTGAGCACCGCCATCCACGCCGACCAGCTGACCCCTGCCTGGAGAATCTACAGCACCGGCAATAACGTGTTCCAGACCCAGGC |
| | CGGCTGCCTGATTGGCGCCGAGCACGTGGACACCAGCTACGAGTGTGATATCCCCATCGGCGCTGGCATCTGCGCCAGCTACCACACAGTGAGCCTGCTGCGGTCCACCAGCCAGAAAAGCA |
| | TCGTGGCCTACACCATGAGCCTGGGCGCCGACAGCAGCATCGCCTACAGCAATAATACCATCGCCATCCCTACAAACTTTTCCATTAGCATCACCACCGAGGTGATGCCTGTGTCCATGGCC |
| | AAGACCTCCGTGGACTGCAACATGTACATTTGCGGCGATTCTACAGAGTGCGCCAACCTGCTGCTGCAGTACGGCTCCTTCTGCACCCAGCTGAATCGGGCCCTGAGCGGCATCGCCGCCGA |
| | ACAGGATAGAAATACCCGGGAGGTGTTCGCCCAGGTGAAGCAGATGTACAAGACCCCCACCCTGAAGTACTTCGGCGGCTTCAACTTCAGCCAGATCCTGCCAGATCCCCTGAAGCCCACCA |
| | AGCGGTCCTTTATCGAGGACCTGCTGTTCAACAAGGTGACACTGGCCGACGCCGGCTTCATGAAACAGTACGGCGAGTGCCTGGGCGACATCAATGCCCGGGACCTGATCTGCGCCCAGAAG |
| | TTCAATGGCCTGACCGTGCTGCCCCCCCTGCTGACCGACGACATGATCGCTGCCTACACAGCCGCCCTGGTGAGCGGGACCGCCACCGCCGGCTGGACATTTGGCGCCGGCGCCGCCCTGCA |
| | GATCCCTTTCGCCATGCAGATGGCCTACCGGTTCAACGGCATCGGCGTGACCCAGAACGTGCTGTACGAGAATCAGAAGCAGATCGCCAACCAGTTCAATAAGGCTATCAGCCAGATCCAGG |
| | AGAGCCTGACAACCACCTCCACCGCCCTGGGCAAGCTGCAGGACGTGGTGAACCAGAATGCCCAGGCCCTGAATACCCTGGTGAAGCAGCTGAGCAGCAATTTTGGCGCCATCAGCTCTGTG |
| | CTGAACGACATCCTGTCTCGGCTGGACCCCCCAGAGGCCGAGGTGCAGATTGACAGGCTGATCACCGGCCGGCTGCAGAGCCTGCAGACCTACGTGACCCAGCAGCTGATCCGGGCCGCCGA |
| | GATCCGGGCCAGCGCCAACCTGGCCGCCACCAAGATGTCCGAGTGTGTGCTGGGCCAGAGCAAGCGGGTGGACTTCTGCGGCAAGGGCTACCACCTGATGTCCTTCCCCCAGGCCGCCCCTC |
| | ACGGCGTGGTGTTCCTGCACGTGACCTACGTGCCCAGCCAGGAGAGAAATTTCACCACCGCCCCCGCCATCTGCCACGAGGGCAAGGCATACTTTCCCAGAGAGGGCGTGTTCGTGTTCAAT |
| | GGCACCTCCTGGTTCATCACCCAGCGGAATTTCTTCAGCCCCCAGATCATCACAACCGACAACACCTTCGTGAGCGGCAATTGTGACGTGGTGATCGGCATCATTAACAATACCGTGTACGA |
| | CCCTCTGCAGCCCGAGCTGGACAGCTTCAAGGAGGAGCTGGACAAGTACTTCAAGAATCATACCAGCCCCGACGTGGACCTGGGCGACATCAGCGGCATCAACGCCAGCGTGGTGAATATCC |
| | AGAAGGAAATCGACAGACTGAATGAGGTGGCCAAGAATCTGAATGAGTCCCTGATCGACCTGCAGGAGCTGGGCAAGTACGAGCAGTACATCAAGTGGCCCTGGTACGTGTGGCTGGGCTTT |
| | ATCGCCGGCCTGATCGCCATCGTGATGGTGACCATCCTGCTGTGCTGCATGACCAGCTGCTGCAGCTGTCTGAAGGGCGCCTGTAGCTGCGGCTCCTGCTGCAAGTTCGATGAGGACGACAG |
| | CGAGCCTGTGCTGAAGGGCGTGAAGCTGCACTACTAA (SEQ ID NO: 570) |
| KHOSTA2_S2P | ATGGACTCTAAGGGCAGCAGCCAGAAGGGCAGCCGGCTGCTGCTGCTGCTGGTGGTGTCCAACCTGCTGCTGCCTCAGGGCGTGGTGGGCCAGCAGGACGGCTGTGCCACCCTGAGCACCAA |
| | GCTGACCCCTACCCAGCTGCAGGTGAACTCCACCCGCCGGGGATTTTACTACTTCGACGATATCTACAGAAGCAGCCTGCGGGTGCTGGTGCCTGGCTACTTCCTGCCCTTCGGCACCAATA |
| | TCACCCGGTACCAGGCCCAGGTGATCAATTGGACCGCCATCTTCTTCGACAACCCTGTGATCCCTTTCAACGATGGCGTGTACTTCGCTGCCCTGGAGCACAGCAATGTGGTGAGAGGCTGG |
| | ATCTTCGGCACAACCCTGGACAATACCACCCAGTCCGCCATCGTGGTGAATAACGCCACACACATCCTGATCAGCGTGTGTTACTTCAACGTGTGCAAGGAGCCCATGTTCGCCGTGAGCAA |
| | TTTCAAGCCCTACAAGAGCTGGGTGTACGACAGAGCCAATAACTGCACCTTCAACCGGGCCTACCCTCTGAATATCAGCACCACACCCGAGCCAGGCAAGTTTAAGGCCCTGCGGGAGCAGG |
| | TGTTCCGGTACCAGGACGGCTTTCTGTACATCTACCACAGCTACGAGAGCATCAATAGCGATACACTGCCTCCCGGCTTCAGCGTGCTGAAGCCTATGCTGAAGCTGCCCCTGGGCCTGAAC |
| | ATCACCAGATTCCGGGTGGTGATGGCCATGCACAGCCTGACAACAAGCAATTTCAATACCCACAGCGTGAACTACTTCGTGGGCCACCTGAAGCCTCTGACCATGCTGGTGGAGTTCAGCCC |
| | CAATGGCACCATCATTGATGCCATCGACTGCAGCCAGGACCCTCTGAGCGAGCTGAAGTGCACCACCAAGAGCTTCAGCGTGGAGAAGGGCATCTACCAGACCAGCAATTTCAGAGTGAGCC |
| | CCAGCCTGGACGTGGTGCGGTTCCCAAATATGACAAATATCTGCCCTTTCGACCAGGTGTTTAACAAGACCCAGTTTCCCAGCGTGTACGCCTGGGAGCGGGTCCGGATCAGCGATTGCGTG |
| | AGCGACTACACAGTGCTGTACAACTCCAGCGCCAGCTTCAGCACCTTCAAGTGCTACGGCGTGAGCCCCACCAAGCTGAACGACCTGTGCTTCAGCGGCGTGTACGCCGACTACTTCGTGGT |
| | GAAAGGCGACCACGTGCACCAGATCGCCCCCGGCCAGACAGGCGTGATCGCCGATTATAATTACAAGCTGCCTAGCGAGTTCGTGGGCTGTATCCTGGCCTGGAATACCAGAACCATCGACA |
| | GCAAGAGAGGCTTCTACTACAGACTGTTCAGACACGGCAACATCAGACCCTACGAGAGAGACACCTCTAATGTGCCCTACAACGCCGCCGGCGGCACCTGCAATCAGCCCGGCACCCACAAT |
| | TGCTACGAGCCTCTGCAGGACTACGGATTCACCAGCACCTCCGGCGTGGGCTACCAGCCTTTCAGAGTGGTGGTGCTGAGCTTCGAACTGCTGAACGCCCCCGCCACCGTGTGTGGCCCTAA |
| | GCAGTCCACCGACCTGGTGAAAAACAAGTGTGTGAATTTCAATTTCAATGGCCTGACCGGCACCGGCGTGCTGACCGACAGCAACAAGAAGTTCCAGCCCTTCCAGCAGTTCGGCCGGGACA |
| | GCGCCGACTTCACCGACAGCGTGAAGGACCCCAAGACACTGGAGATCCTGGACATCACCCCTTGTAGCTATGGCGGCGTGAGCGTGATCACACCTGGCACCAATACCAGCGACTCCGTGGCC |
| | GTGCTGTACCAGGACGTGAATTGTACCGACGTGCCCACCATGCTGCACATGGACCAGGTGAGCAATGATTGGAGAGTGTACGCCGTGAATACAGACGGCAACATGTTTCAGACCCAGGCCGG |
| | CTGCCTGGTGGGCGCCACCTACGACAATACCTCCTACGAGTGCGACATCCCCGTGGGCGCCGGCGTGTGTGCCAAGTTCCAGACCACCACAAGAGCCAAACAGTCTAGCATCCTGGCCTACA |
| | CCATGAGCCTGGGCGAGGACAGCAACGTGGCCTACAGCAATAACAGCATCGCCATCCCTACCAATTTTACCATTAGCGTGACCACCGAGGTGCTGCCCGTGAGCATGACCAAGACCGCCGTG |
| | GACTGCAATATGTACATCTGCGGCGACTCTACCGAGTGCAGCAGCCTGCTGCTGCAGTACGGCAGCTTCTGCCAGCAGCTGAATAGAGCCCTGGCCGGAGTGAGCGTGGAACAGGACAAGAA |
| | CACACAGGACGTGTTCGCCCAGGTGAAAAGCATCTACAAGGTGAGCGCCATCAAGGACTTCGGCGGATTCAACTTCTCTCAGATCCTGCCTGACCCTGCCAAGCCCAGCAAGAGATCCTTCA |
| | TCGAAGACCTGCTGTACAACAAGGTGACCCTGGCCGACCCTGGCTTCATGAAGCAGTACGGCGACTGCCTGGGCGGCGTGAATGCCCGGGACCTGATCTGCGCCCAGAAGTTCAATGGCCTG |
| | ACAGTGCTGCCCCCTCTGCTGACCGACGACATGATCGCCGCCTACACAAGCGCCCTGGTGAGCGGCACCGCCCTGAGCGGATTCACCTTCGGCGCCGGCGCTGCCCTGCAGATCCCTTTTGC |
| | CATGCAGATGGCCTACAGATTCAATGGCATCGGCGTGACACAGAATGTGCTGTACGAGAATCAGAAGCAGATCGCCAACCAGTTCAATAAGGCCATCAGCCAGATCCAGGAGAGCCTGACCA |
| | CAACAGCCACCGCCCTGGGCAAGCTGCAGGACGTGATCAACCAGAATGCTATCGCCCTGAATACCCTGGTGAAGCAGCTGTCCAGCAATTTCGGCGCCATCAGCAGCGTGCTGAATGACATC |
| | CTGAGCAGACTGGACCCCCCCGAGGCCGAGGTGCAGATCGACAGACTGATCACCGGACGGCTGCAGAGCCTGCAGACCTACGTGACCCAGCAGCTGATCAGAGCCGCCGACATCCGGGCCAG |
| | CGCCAATCTGGCCGCCACCAAGATGAGCGAATGTGTGCTGGGCCAGTCTAAGCGGGTGGACTTCTGTGGCAAGGGCTACCACCTGATGTCTTTCCCTCAGGCCGCCCCCCACGGCGTGGTGT |
| | TCCTGCACGTGACATACGTGCCCAGCCAGGATAAGAACTTCACCACCGCCCCCGCCATCTGCCACAAGGGCAAGGCCTACTTCCCCAGAGAGGGCGTGTTCGTGACCAATGGCACCCACTG |
| | GTTCGTGACACAGCGGAACTTCTACCAGCCCGAGGTGATCACCACCGAGAACACATTCGAGAGCGGCAATTGTGACGTGGTGATCGGCATCGTGAACAATACCGTGTACGACCCTCTGCAGC |
| | CCGAGCTGGAGAGCTTCAAGGAGGAGCTGGACAAGTACTTCAAGAATCACACCTCCCCCGACGTGGACTTCGACGACATCTCTGGCATCAACGCTAGCGTGGTGGACATTAAGAAGGAGATT |
| | ATGACGACCACAGCGAGCCCGTGCTGAAGGGAGTGAAGCTGCACTACTAAGAGCACCTGAACGAGATCGCCAAGAGCCTGAACGAGAGCCTGATCGACCTGCAGGAGCTGGGCAAGTACGAA |
| | CAGTACATCAAGTGGCCCTGGTACGTGTGGCTGGGCTTCATCGCCGGCCTGATCGCCATCATCATGGCCACAATCATGCTGTGCTGCATGACCAGCTGCTGCAGCTGCCTGAAGGGCCTGTG |
| | TTCTTGCGGCTCCTGCTGTAAGTTCG (SEQ ID NO: 571) |
| BQ1.1_S2P | ATGGATTCCAAGGGCAGCAGCCAGAAGGGCAGCCGGCTGCTGCTGCTGCTGGTGGTGAGCAACCTGCTGCTGCCTCAGGGGGTGGTGGGACAGTCCAGCCAGTGCGTGAACCTGATCACCAG |
| | AACCCAGAGCTACACAAATTCTTTCACCCGGGGGGTGTACTACCCCGATAAAGTGTTCAGAAGCTCCGTGCTGCACAGCACCCAGGACCTCTTCCTGCCCTTTTTCAGCAATGTGACCTGGT |
| | TCCATGCTATCAGCGGCACCAACGGCACCAAGAGATTCGACAACCCTGTGCTGCCCTTCAATGACGGCGTGTACTTCGCCAGCACCGAGAAAAGCAATATCATCAGAGGCTGGATCTTCGGC |
| | ACCACACTGGACAGCAAGACCCAGAGCCTGCTGATCGTGAACAACGCAACCAACGTGGTGATCAAGGTGTGCGAGTTCCAGTTCTGCAACGACCCCTTCCTGGACGTGTACTACCACAAGAA |
| | CAATAAGAGCTGGATGGAGAGCGAATTCCGGGTGTACTCCAGCGCCAATAATTGCACCTTCGAGTACGTGAGCCAGCCCTTCCTGATGGACCTGGAGGGCAAGCAGGGCAACTTCAAGAATC |
| | TGCGGGAATTCGTGTTCAAGAACATCGACGGCTACTTCAAGATCTACAGCAAGCACACCCCTATCAACCTGGGCCGGGACCTGCCCCAGGGCTTCTCCGCCCTGGAGCCTCTGGTGGACCTG |
| | CCCATCGGCATCAATATTACCCGGTTCCAGACACTGCTGGCCCTGCACAGGAGCTACCTGACCCCAGGCGACAGCAGCAGCGGATGGACAGCCGGCGCCGCTGCCTACTACGTGGGCTACCT |
| | GCAGCCCCGGACCTTCCTGCTGAAGTACAATGAGAACGGCACCATCACAGACGCCGTGGACTGCGCCCTGGACCCACTGAGCGAAACCAAGTGCACACTGAAGTCCTTCACAGTGGAGAAGG |
| | GCATCTACCAGACAAGCAATTTCCGGGTGCAGCCAACCGAGAGCATCGTGAGATTCCCTAATATCACCAACCTGTGCCCTTTTGACGAAGTGTTCAATGCCACCACCTTCGCCAGCGTGTAC |
| | GCCTGGAATCGGAAGAGAATCAGCAACTGCGTGGCCGACTACAGCGTGCTGTACAATTTTGCCCCTTTCTTCGCCTTCAAGTGCTACGGCGTGAGCCCCACAAAGCTGAATGACCTGTGCTT |
| | CACCAATGTGTACGCCGACAGCTTCGTGATCCGGGGCAACGAGGTGAGCCAGATCGCCCCTGGCCAGACAGGCAACATCGCAGATTACAATTACAAGCTGCCCGACGACTTCACCGGCTGTG |
| | TGATCGCCTGGAATTCTAATAAGCTGGACAGCACCGTGGGCGGCAACTACAACTACCGGTACAGACTGTTCCGCAAGAGCAAGCTGAAGCCCTTCGAGCGGGACATCAGCACCGAGATCTAC |
| | CAGGCCGGCAACAAGCCCTGCAATGGCGTGGCCGGCGTGAACTGCTACTTCCCCCTGCAGAGCTACGGCTTCAGACCCACCTACGGCGTGGGCCACCAGCCTTACAGAGTGGTGGTGCTGAG |
| | CTTCGAGCTGCTGCATGCCCCCGCCACCGTGTGCGGCCCCAAAAAGAGCACCAACCTGGTGAAGAATAAGTGTGTGAACTTCAATTTCAATGGCCTGACCGGCACCGGCGTGCTGACCGAGA |
| | GCAATAAGAAGTTCCTGCCCTTCCAGCAGTTCGGCCGGGATATCGCCGACACAACCGACGCCGTGCGGGATCCTCAGACCCTGGAGATTCTGGACATCACCCCTTGCTCCTTCGGCGGCGTG |
| | AGCGTGATCACCCCCGGCACAAATACCTCCAATCAGGTGGCCGTGCTGTACCAGGGCGTGAATTGCACAGAAGTGCCCGTGGCCATCCACGCCGACCAGCTGACCCCCACCTGGAGAGTGTA |
| | CAGCACAGGGAGCAACGTGTTCCAGACCCGGGCCGGCTGCCTGATTGGCGCCGAGTACGTGAATAACAGCTACGAGTGCGACATCCCCATCGGCGCCGGCATCTGCGCCAGCTACCAGACCC |
| | AGACAAAGAGCCACGGCAGCGCCAGCTCCGTGGCCAGCCAGTCTATCATCGCCTACACCATGAGCCTGGGCGCCGAGAATAGCGTGGCCTACTCCAACAATAGCATCGCCATCCCCACCAAT |
| | TTCACCATCAGCGTGACCACCGAGATTCTGCCCGTGAGCATGACAAAGACAAGCGTGGACTGTACCATGTACATCTGCGGCGACAGCACCGAGTGCAGCAACCTGCTGCTGCAGTACGGCAG |
| | CTTCTGCACCCAGCTGAAGCGCGCCCTGACCGGCATCGCCGTGGAGCAGGACAAGAACACCCAGGAGGTGTTCGCCCAGGTGAAGCAGATCTACAAGACCCCCCCCATCAAGTACTTCGGCG |
| | GCTTCAATTTCTCTCAGATCCTGCCCGACCCCTCCAAGCCTAGCAAGCGGTCCTTCATCGAGGACCTGCTGTTCAATAAGGTGACACTGGCCGATGCCGGCTTCATCAAGCAGTACGGCGAC |
| | TGCCTGGGCGATATCGCCGCCCGGGACCTGATCTGCGCCCAGAAGTTCAACGGCCTGACAGTGCTGCCCCCCCTGCTGACCGACGAGATGATCGCCCAGTACACCAGCGCCCTCCTGGCCGG |
| | CACCATCACCTCTGGCTGGACATTCGGCGCTGGCGCCGCCCTGCAGATCCCTTTCGCCATGCAGATGGCCTACCGGTTCAATGGGATCGGAGTGACCCAGAATGTGCTGTACGAAAATCAGA |
| | AGCTGATCGCCAACCAGTTCAATAGCGCCATCGGCAAGATCCAGGACAGCCTGAGCAGCACCGCCTCCGCCCTGGGCAAGCTGCAGGACGTGGTGAACCACAATGCCCAGGCCCTGAACACC |
| | CTGGTGAAGCAGCTGAGCAGCAAGTTCGGCGCCATCAGCAGCGTGCTGAATGACATCCTGAGCCGGCTGGACCCTCCTGAGGCCGAGGTGCAGATCGACCGGCTGATCACCGGCAGACTGCA |
| | GAGCCTGCAGACATACGTGACCCAGCAGCTGATCCGGGCCGCCGAGATCCGGGCCTCCGCCAATCTGGCCGCCACCAAGATGTCTGAGTGCGTGCTGGGCCAGAGCAAGCGGGTGGACTTCT |
| | GCGGGAAGGGCTACCACCTGATGAGCTTCCCTCAGTCCGCCCCTCACGGCGTGGTGTTCCTGCACGTGACATACGTGCCCGCCCAGGAGAAGAATTTCACCACCGCCCCAGCCATCTGCCAC |
| | GACGGCAAGGCCCACTTTCCCAGAGAAGGCGTGTTCGTGAGCAACGGCACCCACTGGTTCGTGACCCAGAGAAATTTCTACGAGCCCCAGATCATCACCACCGACAATACCTTCGTGAGCGG |
| | CAACTGTGATGTGGTGATCGGCATCGTGAATAACACCGTGTACGACCCTCTGCAGCCTGAGCTGGACTCCTTCAAGGAGGAGCTGGACAAGTACTTCAAGAACCACACCAGCCCTGATGTGG |
| | ACCTGGGCGATATCTCCGGCATCAACGCCAGCGTGGTGAATATTCAGAAGGAGATCGACAGACTGAACGAGGTGGCCAAGAACCTGAACGAGAGCCTGATCGACCTGCAGGAACTGGGCAAG |
| | TACGAGCAGTACATCAAGTGGCCATGGTACATCTGGCTGGGCTTCATCGCCGGCCTGATCGCCATCGTGATGGTGACAATCATGCTGTGCTGCATGACCTCCTGCTGCTCCTGCCTGAAGGG |
| | CTGTTGCAGCTGCGGCAGCTGCTGCAAGTTCGACGAGGACGATTCCGAGCCCGTGCTGAAGGGCGTGAAGCTGCACTACTAA (SEQ ID NO: 569) |
| SARS_S2P | ATGGATTCCAAGGGCAGCTCCCAGAAGGGCTCCCGGCTGCTGCTGCTGCTGGTGGTGTCCAACCTGCTGCTGCCTCAGGGCGTGGTGGGCCAGAGCGACCTGGACAGATGCACAACCTTTGA |
| | TGACGTGCAGGCCCCCAACTACACACAGCACACCAGCAGCATGCGGGGCGTGTACTACCCTGACGAGATCTTCCGGAGCGACACCCTGTACCTGACACAGGACCTCTTCCTGCCCTTCTACA |
| | GCAACGTGACCGGCTTCCACACCATCAACCACACCTTCGGCAATCCTGTGATCCCCTTCAAGGACGGCATCTACTTCGCTGCCACCGAGAAGTCCAATGTGGTGCGGGGCTGGGTGTTCGGC |
| | AGCACCATGAATAATAAGAGCCAGAGCGTGATCATCATCAATAATAGCACCAATGTGGTGATCAGGGCCTGCAATTTCGAGCTGTGCGACAATCCCTTCTTCGCCGTGAGCAAGCCTATGGG |
| | CACCCAGACCCACACCATGATCTTCGACAATGCCTTCAATTGCACCTTCGAGTACATCAGCGACGCCTTTAGCCTGGACGTGTCCGAAAAAAGCGGCAATTTCAAGCACCTGCGGGAGTTCG |
| | TGTTCAAGAATAAGGACGGCTTTCTGTACGTGTACAAGGGCTACCAGCCCATCGACGTGGTGCGCGACCTGCCCAGCGGCTTCAATACCCTGAAGCCCATCTTCAAGCTGCCCCTGGGCATC |
| | AATATCACCAACTTTCGGGCCATCCTGACCGCCTTCAGCCCTGCCCAGGATATCTGGGGCACCAGCGCCGCCGCCTACTTCGTGGGCTACCTGAAGCCCACAACATTCATGCTGAAGTACGA |
| | CGAGAATGGCACCATCACCGACGCCGTGGACTGCAGCCAGAATCCTCTGGCCGAGCTGAAGTGCAGCGTGAAGTCCTTTGAGATCGATAAGGGAATCTACCAGACCAGCAACTTCCGGGTGG |
| | TGCCTAGCGGCGACGTGGTGCGGTTCCCTAATATCACCAACCTGTGCCCCTTCGGCGAGGTGTTCAATGCCACCAAGTTCCCCTCCGTGTACGCCTGGGAGAGAAAGAAGATCAGCAATTGC |
| | GTGGCCGACTACAGCGTGCTGTACAATTCTACCTTCTTCTCCACCTTCAAGTGCTACGGCGTGTCTGCCACCAAGCTGAACGACCTGTGCTTTTCCAACGTGTACGCCGACAGCTTCGTGGT |
| | GAAGGGCGACGACGTGCGGCAGATCGCCCCTGGCCAGACCGGCGTGATCGCTGATTACAACTACAAGCTGCCAGATGACTTCATGGGCTGCGTGCTGGCCTGGAACACCAGAAATATCGACG |
| | CCACCAGCACCGGAAACTACAACTACAAGTACCGGTACCTGCGGCACGGCAAGCTGCGGCCCTTTGAGCGGGACATCAGCAACGTGCCTTTCAGCCCTGACGGAAAGCCCTGTACCCCCCCC |
| | GCCCTGAACTGCTACTGGCCTCTGAATGACTACGGCTTCTACACCACAACCGGCATCGGCTACCAGCCTTACAGAGTGGTGGTGCTGAGCTTCGAGCTGCTGAATGCCCCTGCCACCGTGTG |
| | CGGCCCAAAGCTGAGCACCGACCTGATCAAGAACCAGTGCGTGAACTTCAATTTCAACGGCCTGACCGGCACCGGCGTGCTGACCCCCAGCAGCAAGAGATTCCAGCCTTTCCAGCAGTTCG |
| | GCAGAGACGTGAGCGACTTCACAGACAGCGTGCGGGACCCCAAGACATCTGAGATCCTGGATATCAGCCCCTGCGCCTTCGGCGGAGTGTCCGTGATTACCCCTGGCACCAACGCCAGCAGC |
| | GAGGTGGCCGTGCTGTACCAGGACGTGAATTGTACCGACGTGAGCACCGCCATCCACGCCGACCAGCTGACCCCTGCCTGGAGAATCTACAGCACCGGCAATAACGTGTTCCAGACCCAGGC |
| | CGGCTGCCTGATTGGCGCCGAGCACGTGGACACCAGCTACGAGTGTGATATCCCCATCGGCGCTGGCATCTGCGCCAGCTACCACACAGTGAGCCTGCTGCGGTCCACCAGCCAGAAAAGCA |
| | TCGTGGCCTACACCATGAGCCTGGGCGCCGACAGCAGCATCGCCTACAGCAATAATACCATCGCCATCCCTACAAACTTTTCCATTAGCATCACCACCGAGGTGATGCCTGTGTCCATGGCC |
| | AAGACCTCCGTGGACTGCAACATGTACATTTGCGGCGATTCTACAGAGTGCGCCAACCTGCTGCTGCAGTACGGCTCCTTCTGCACCCAGCTGAATCGGGCCCTGAGCGGCATCGCCGCCGA |
| | ACAGGATAGAAATACCCGGGAGGTGTTCGCCCAGGTGAAGCAGATGTACAAGACCCCCACCCTGAAGTACTTCGGCGGCTTCAACTTCAGCCAGATCCTGCCAGATCCCCTGAAGCCCACCA |
| | AGCGGTCCTTTATCGAGGACCTGCTGTTCAACAAGGTGACACTGGCCGACGCCGGCTTCATGAAACAGTACGGCGAGTGCCTGGGCGACATCAATGCCCGGGACCTGATCTGCGCCCAGAAG |
| | TTCAATGGCCTGACCGTGCTGCCCCCCCTGCTGACCGACGACATGATCGCTGCCTACACAGCCGCCCTGGTGAGCGGGACCGCCACCGCCGGCTGGACATTTGGCGCCGGCGCCGCCCTGCA |
| | GATCCCTTTCGCCATGCAGATGGCCTACCGGTTCAACGGCATCGGCGTGACCCAGAACGTGCTGTACGAGAATCAGAAGCAGATCGCCAACCAGTTCAATAAGGCTATCAGCCAGATCCAGG |
| | AGAGCCTGACAACCACCTCCACCGCCCTGGGCAAGCTGCAGGACGTGGTGAACCAGAATGCCCAGGCCCTGAATACCCTGGTGAAGCAGCTGAGCAGCAATTTTGGCGCCATCAGCTCTGTG |
| | CTGAACGACATCCTGTCTCGGCTGGACCCCCCAGAGGCCGAGGTGCAGATTGACAGGCTGATCACCGGCCGGCTGCAGAGCCTGCAGACCTACGTGACCCAGCAGCTGATCCGGGCCGCCGA |
| | GATCCGGGCCAGCGCCAACCTGGCCGCCACCAAGATGTCCGAGTGTGTGCTGGGCCAGAGCAAGCGGGTGGACTTCTGCGGCAAGGGCTACCACCTGATGTCCTTCCCCCAGGCCGCCCCTC |
| | ACGGCGTGGTGTTCCTGCACGTGACCTACGTGCCCAGCCAGGAGAGAAATTTCACCACCGCCCCCGCCATCTGCCACGAGGGCAAGGCATACTTTCCCAGAGAGGGCGTGTTCGTGTTCAAT |
| | GGCACCTCCTGGTTCATCACCCAGCGGAATTTCTTCAGCCCCCAGATCATCACAACCGACAACACCTTCGTGAGCGGCAATTGTGACGTGGTGATCGGCATCATTAACAATACCGTGTACGA |
| | CCCTCTGCAGCCCGAGCTGGACAGCTTCAAGGAGGAGCTGGACAAGTACTTCAAGAATCATACCAGCCCCGACGTGGACCTGGGCGACATCAGCGGCATCAACGCCAGCGTGGTGAATATCC |
| | AGAAGGAAATCGACAGACTGAATGAGGTGGCCAAGAATCTGAATGAGTCCCTGATCGACCTGCAGGAGCTGGGCAAGTACGAGCAGTACATCAAGTGGCCCTGGTACGTGTGGCTGGGCTTT |
| | ATCGCCGGCCTGATCGCCATCGTGATGGTGACCATCCTGCTGTGCTGCATGACCAGCTGCTGCAGCTGTCTGAAGGGCGCCTGTAGCTGCGGCTCCTGCTGCAAGTTCGATGAGGACGACAG |
| | CGAGCCTGTGCTGAAGGGCGTGAAGCTGCACTACTAA (SEQ ID NO: 570) |
| KHOSTA2_S2P | ATGGACTCTAAGGGCAGCAGCCAGAAGGGCAGCCGGCTGCTGCTGCTGCTGGTGGTGTCCAACCTGCTGCTGCCTCAGGGCGTGGTGGGCCAGCAGGACGGCTGTGCCACCCTGAGCACCAA |
| | GCTGACCCCTACCCAGCTGCAGGTGAACTCCACCCGCCGGGGATTTTACTACTTCGACGATATCTACAGAAGCAGCCTGCGGGTGCTGGTGCCTGGCTACTTCCTGCCCTTCGGCACCAATA |
| | TCACCCGGTACCAGGCCCAGGTGATCAATTGGACCGCCATCTTCTTCGACAACCCTGTGATCCCTTTCAACGATGGCGTGTACTTCGCTGCCCTGGAGCACAGCAATGTGGTGAGAGGCTGG |
| | ATCTTCGGCACAACCCTGGACAATACCACCCAGTCCGCCATCGTGGTGAATAACGCCACACACATCCTGATCAGCGTGTGTTACTTCAACGTGTGCAAGGAGCCCATGTTCGCCGTGAGCAA |
| | TTTCAAGCCCTACAAGAGCTGGGTGTACGACAGAGCCAATAACTGCACCTTCAACCGGGCCTACCCTCTGAATATCAGCACCACACCCGAGCCAGGCAAGTTTAAGGCCCTGCGGGAGCAGG |
| | TGTTCCGGTACCAGGACGGCTTTCTGTACATCTACCACAGCTACGAGAGCATCAATAGCGATACACTGCCTCCCGGCTTCAGCGTGCTGAAGCCTATGCTGAAGCTGCCCCTGGGCCTGAAC |
| | ATCACCAGATTCCGGGTGGTGATGGCCATGCACAGCCTGACAACAAGCAATTTCAATACCCACAGCGTGAACTACTTCGTGGGCCACCTGAAGCCTCTGACCATGCTGGTGGAGTTCAGCCC |
| | CAATGGCACCATCATTGATGCCATCGACTGCAGCCAGGACCCTCTGAGCGAGCTGAAGTGCACCACCAAGAGCTTCAGCGTGGAGAAGGGCATCTACCAGACCAGCAATTTCAGAGTGAGCC |
| | CCAGCCTGGACGTGGTGCGGTTCCCAAATATGACAAATATCTGCCCTTTCGACCAGGTGTTTAACAAGACCCAGTTTCCCAGCGTGTACGCCTGGGAGCGGGTCCGGATCAGCGATTGCGTG |
| | AGCGACTACACAGTGCTGTACAACTCCAGCGCCAGCTTCAGCACCTTCAAGTGCTACGGCGTGAGCCCCACCAAGCTGAACGACCTGTGCTTCAGCGGCGTGTACGCCGACTACTTCGTGGT |
| | GAAAGGCGACCACGTGCACCAGATCGCCCCCGGCCAGACAGGCGTGATCGCCGATTATAATTACAAGCTGCCTAGCGAGTTCGTGGGCTGTATCCTGGCCTGGAATACCAGAACCATCGACA |
| | GCAAGAGAGGCTTCTACTACAGACTGTTCAGACACGGCAACATCAGACCCTACGAGAGAGACACCTCTAATGTGCCCTACAACGCCGCCGGCGGCACCTGCAATCAGCCCGGCACCCACAAT |
| | TGCTACGAGCCTCTGCAGGACTACGGATTCACCAGCACCTCCGGCGTGGGCTACCAGCCTTTCAGAGTGGTGGTGCTGAGCTTCGAACTGCTGAACGCCCCCGCCACCGTGTGTGGCCCTAA |
| | GCAGTCCACCGACCTGGTGAAAAACAAGTGTGTGAATTTCAATTTCAATGGCCTGACCGGCACCGGCGTGCTGACCGACAGCAACAAGAAGTTCCAGCCCTTCCAGCAGTTCGGCCGGGACA |
| | GCGCCGACTTCACCGACAGCGTGAAGGACCCCAAGACACTGGAGATCCTGGACATCACCCCTTGTAGCTATGGCGGCGTGAGCGTGATCACACCTGGCACCAATACCAGCGACTCCGTGGCC |
| | GTGCTGTACCAGGACGTGAATTGTACCGACGTGCCCACCATGCTGCACATGGACCAGGTGAGCAATGATTGGAGAGTGTACGCCGTGAATACAGACGGCAACATGTTTCAGACCCAGGCCGG |
| | CTGCCTGGTGGGCGCCACCTACGACAATACCTCCTACGAGTGCGACATCCCCGTGGGCGCCGGCGTGTGTGCCAAGTTCCAGACCACCACAAGAGCCAAACAGTCTAGCATCCTGGCCTACA |
| | CCATGAGCCTGGGCGAGGACAGCAACGTGGCCTACAGCAATAACAGCATCGCCATCCCTACCAATTTTACCATTAGCGTGACCACCGAGGTGCTGCCCGTGAGCATGACCAAGACCGCCGTG |
| | GACTGCAATATGTACATCTGCGGCGACTCTACCGAGTGCAGCAGCCTGCTGCTGCAGTACGGCAGCTTCTGCCAGCAGCTGAATAGAGCCCTGGCCGGAGTGAGCGTGGAACAGGACAAGAA |
| | CACACAGGACGTGTTCGCCCAGGTGAAAAGCATCTACAAGGTGAGCGCCATCAAGGACTTCGGCGGATTCAACTTCTCTCAGATCCTGCCTGACCCTGCCAAGCCCAGCAAGAGATCCTTCA |
| | CCTGATGTCTTTCCCTCAGGCCGCCCCCCACGGCGTGGTGTTCCTGCACGTGACATACGTGCCCAGCCAGGATAAGAACTTCACCACCGCCCCCGCCATCTGCCACAAGGGCAAGGCCTACT |
| | TCCCTCGAAGACCTGCTGTACAACAAGGTGACCCTGGCCGACCCTGGCTTCATGAAGCAGTACGGCGACTGCCTGGGCGGCGTGAATGCCCGGGACCTGATCTGCGCCCAGAAGTTCAATGG |
| | CCTGACAGTGCTGCCCCCTCTGCTGACCGACGACATGATCGCCGCCTACACAAGCGCCCTGGTGAGCGGCACCGCCCTGAGCGGATTCACCTTCGGCGCCGGCGCTGCCCTGCAGATCCCTT |
| | TTGCCATGCAGATGGCCTACAGATTCAATGGCATCGGCGTGACACAGAATGTGCTGTACGAGAATCAGAAGCAGATCGCCAACCAGTTCAATAAGGCCATCAGCCAGATCCAGGAGAGCCTG |
| | ACCACAACAGCCACCGCCCTGGGCAAGCTGCAGGACGTGATCAACCAGAATGCTATCGCCCTGAATACCCTGGTGAAGCAGCTGTCCAGCAATTTCGGCGCCATCAGCAGCGTGCTGAATGA |
| | CATCCTGAGCAGACTGGACCCCCCCGAGGCCGAGGTGCAGATCGACAGACTGATCACCGGACGGCTGCAGAGCCTGCAGACCTACGTGACCCAGCAGCTGATCAGAGCCGCCGACATCCGGG |
| | CCAGCGCCAATCTGGCCGCCACCAAGATGAGCGAATGTGTGCTGGGCCAGTCTAAGCGGGTGGACTTCTGTGGCAAGGGCTACCACAGAGAGGGCGTGTTCGTGACCAATGGCACCCACTGG |
| | TTCGTGACACAGCGGAACTTCTACCAGCCCGAGGTGATCACCACCGAGAACACATTCGAGAGCGGCAATTGTGACGTGGTGATCGGCATCGTGAACAATACCGTGTACGACCCTCTGCAGCC |
| | CGAGCTGGAGAGCTTCAAGGAGGAGCTGGACAAGTACTTCAAGAATCACACCTCCCCCGACGTGGACTTCGACGACATCTCTGGCATCAACGCTAGCGTGGTGGACATTAAGAAGGAGATTG |
| | AAGCACCTGAACGAGATCGCCAAGAGCCTGAACGAGAGCCTGATCGACCTGCAGGAGCTGGGCAAGTACGAACAGTACATCAAGTGGCCCTGGTACGTGTGGCTGGGCTTCATCGCCGGCCT |
| | GATCGCCATCATCATGGCCACAATCATGCTGTGCTGCATGACCAGCTGCTGCAGCTGCCTGAAGGGCCTGTGTTCTTGCGGCTCCTGCTGTAAGTTCGTGACGACCACAGCGAGCCCGTGCT |
| | GAAGGGAGTGAAGCTGCACTACTAA (SEQ ID NO: 571) |
| BANAL20247_ | ATGGACAGCAAAGGCAGCAGCCAGAAGGGCTCCAGACTGCTGCTGCTGCTGGTGGTGAGCAACCTGCTGCTGCCCCAGGGCGTGGTGGGCCAGACAACCTGCGTGACCGGACCTACCACCGA |
| S2P | GAACAAGCAGAATGTGAGCAGCCTGATGAGAGGCGTGTACTACCCCGACGATATCTACCGGAGCAACGTGAATGTGCTGTTCACCGTGCCCTTCCTGAGATTCAACAGCACACTGACCTGGT |
| | ACAATTTCTGGAATCAGGCCTATACCTCCAGAGTGATGGAGTTCGGCGACGGCATCTACTTCAGCACCGTGGACAAGAGCAATGCCGTGAGAGGCTGGATCTTCGGCACCACCCTGGACAAT |
| | ACCACACAGAGCGCCCTGCTGATCAATAATGGCAGCCTGGTGACCATCCAGGTGTGCTACTTCCAGTTTTGTGCCAACCCTGCCTTCCTGGTGGCCGGAGGCCAGCAGACCTCTGCCGCCGT |
| | GTACATCAGCTCCCACAATTGCACCTACTCCGAGGTGCTGAGCCACATCAGCCTGGACTTCACAGAGGACACCGGCAGCTTCAAGCGGCTGAGAGAGTTCGTGTTCAAGAATAATGACGGGT |
| | TCCTGCACATCTACGGCGCCTACGAGCCTCACGTGATCGGCATCGGCGTGACCCCCAGCCTGCCTAGCACCTTCAAGCCTCTGATCCCCCTGTGGAAGCTGCCTCTGGGCCTGAATATCACC |
| | AACTTCAAGGTGGTGCTGACCTTCCGGAGCAACAGCCAGCCTCTGCAGGCCAATTTCGCCGTGGGCAGCCTGAAGCTGACAACCTTCATGCTGTCTTTCGACATCAATGGCACCATTGACAA |
| | GGCCGTGGACTGTAGCAGCGACCCCCTGAGCGAGCTGAAGTGCACCCTGAAGTCTTTCAACGTGAGCAAGGGCATCTACCCCACCAGCAACTTTCGGGTGCTGCCTTCCACCGAGGTGGTGC |
| | GCTTCCCCAATATCACCAATTTCTGCCCTTTCGACAAGGTGTTCAATGCCACCAGATTCCCCAACGTGTACGCCTGGCAGCGGACAAAGATCAGCGACTGCATCGCCGACTACACAGTGCTG |
| | TACAATAGCACCAGCTTCAGCACCTTCAAGTGTTACGGCGTGAGCCCTTCCAAACTGATCGACCTGTGCTTCACCAGCGTGTACGCCGACACCTTCCTGATCAGATTCTCCGAGGTGAGACA |
| | GATCGCCCCCGGCGAAACCGGCGTGATCGCCGACTACAATTACAAGCTGCCCGATGACTTTACCGGCTGCGTGCTGGCCTGGAATACCGCCCAGCAGGACATCGGCTCCTACTTCTACCGGA |
| | GCCACCGGGCCGTGAAGCTGAAGCCATTCGAACGGGACCTGAGCTCCGATGAGAACGGCGTGAGAACCCTGAGCACCTACGACTTCAATCCTAACGTGCCCCTGGACTACCAGGCCACCCGG |
| | GTGGTGGTGCTGTCCTTCGAGCTGCTGAATGCCCCAGCTACCGTGTGCGGCCCCAAGCTGAGCACCCAGCTGGTGAAGAACCGGTGTGTGAACTTCAATTTCAACGGCCTGCGGGGCACAGG |
| | CGTGCTGACCGACTCCGACAAGCGGTTCCAGAGCTTCCAGCAGTTCGGCAGAGACAGCGCCGATTTCACCGATAGCGTGCGGGACCCTCAGACCCTGCAGATCCTGGACATCTCCCCCTGCT |
| | CCTTCGGCGGCGTGAGCGTGATCACACCCGGCACCCAGACAAGCAGCAAAGTGGCCGTGCTGTACCAGGACGTGAATTGTACCGATGTGCCAACCGCCCTGGGCCTGGATCAGATCAGCGCC |
| | GCCTGGAGAGTGTACGCCATCGGCAATGACGTGTTCCAGACCCAGGCCGGCTGCCTGGTGGGCGCCGAGCACACCAACATCAGCTACGAGTGCGACATCCCCATCGGCGCCGGCGTGTGCGC |
| | CAGCTACAATTCCCCCGCCGCCCGGGTGGGCACAAATAGCATCATCGCCTACGCCATGAGCATCGGCGCCGAGAGCAGCATCGCCTATTCCAATAACAGCATCGCCATCCCTACAAATTTCT |
| | GCCATCCAGGTGACCACCGAGGTGCTGCCTGTGAGCATGTCCAAGACCAGCGTGGACTGCACCATGTACATCTGCGGCGACAGCCAGGAGTGCAATAAGCTGCTGCTGCAGTACGGCAGCTT |
| | CTGCGCCCAGCTGAATAGGGCCCTGAGCGGCGTGGCCGTGGAGCAGGACAAGAATACCGAGGCCGTGTTCGCCCAGGTGAAGCAGATCTACAAAGCCCCCGTGATCAAGGACCTGGGGGCTT |
| | CAATTTTAGCCAGCTGCTGCCCGACCCTACCAAGCCTAGCCAGAGATCCTTTATCGAGGACCTGCTGTTCGACAAGGTGACCCTGAGCGACGCCGGCTTCATCAAGCAGTACGGCGACTGCC |
| | TGGGCGATATCGCCGCCCGGGACCTGATCTGCGCCCAGAAGTTCAATGGCCTGACAGTGCTGCCTCCTCTGCTGACCGACGAGATGATCGCCGCCTACACAAGCGCCCTGGTGAGCGGCACA |
| | GCCACAGCCGGCTGGACCTTCGGCATCGGCGCCGCCCTGCAGGTGCCCTTCGCCATGCAGATGGCCTACAGATTCAACGGCATCGGCGTGACCCAAATGTGCTGTACGAGAACCAGAAGCTG |
| | ATCGCCAACCAGTTCAATAGCGCCATCGGCAAGATCCAGGATAGCCTGCTGTCCACCGCCTCTGCCCTGGGAAAGCTGCAGGATGTGGTGAATCAGAACGCCCAGGCCCTGAACACCCTGGT |
| | GAAACAGCTGAGCAGCAATTTCGGCGCCATCAGCAGCGTGCTGAACGACATCCTGAGCCGGCTGGACCCCCCTGAGGCCGAGGTGCAGATCGACAGACTGATCACCGGCAGACTGCAGAGCC |
| | TGCAGACCTACGTGACCCAGCAGCTGATCAGAGCCGCCGAGATCCGGGCCAGCGCCAACCTGGCCGCCACCAAGATGAGCGAGTGCGTGCTGGGCCAGAGCAAGAGAGTGGATTTCTGCGGC |
| | AAGGGCTATCACCTGATGAGCTTTCCCCAGTCCGCCCCTCACGGCGTGGTGTTCCTGCACGTGACCTACGTGCCCGCCCAGGAGAAGAATTTCACCACCGCCCCCGCCATTTGCTACGACGG |
| | CAAGGCCCACTTCCCTAGAGAAGGCGTGTTCGTGTCCAACGGAACCCACTGGTTCGTGACCCAGCGGAATTTCTACGAGCCTCAGATCATCACCACCGACAACACCTTCGTGAGCGGCACCT |
| | GCGACGTGGTGATCGGCATTGTGAATAATACCGTGTACGACCCCCTGCAGCCTGAGCTGGACAGCTTCAAGGAGGAGCTGGATAAGTATTTCAAGAATCACACCAGCCCCGACGTGGACCTG |
| | GGCGACATCTCCGGCATCAATGCCAGCGTGGTGAATATCCAGAAGGAGATCGACCGGCTGAATGAGGTGGCCAAAAATCTGAATGAGAGCCTGATCGATCTGCAGGAGCTGGGCAAGTACGA |
| | GCAGTACATCAAGTGGCCCTGGTACATCTGGCTGGGCTTCATCGCCGGCCTGATCGCCATCGTGATGGTGACAATCATGCTGTGCTGTATGACAAGCTGCTGCAGCTGCCTGAAGGGCTGCT |
| | GCAGCTGTGGCAGCTGCTGCAAGTTCGACGAGGACGACAGCGAGCCCGTGCTGAACGGCGTGAAGCTGCACTACTAA (SEQ ID NO: 572) |
| MERS_S2P | ATGGACTCTAAGGGCAGCAGCCAAAAGGGCTCCAGACTGCTGCTCCTCCTGGTTGTGTCCAACCTGCTGCTGCCCCAGGGAGTCGTGGGCCAGTACGTCGATGTGGGCCCAGATTCTGTGAA |
| | GTCCGCCTGTATCGAGGTGGACATTCAGCAGACATTCTTCGACAAGACATGGCCTAGACCCATTGATGTGTCTAAGGCCGACGGGATTATCTATCCCCAGGGAAGGACCTATTCTAACATTA |
| | CAATTACCTATCAGGGCCTGTTTCCCTACCAGGGGGACCACGGCGACATGTATGTCTACTCCGCAGGACACGCCACTGGAACCACACCTCAGAAGCTCTTTGTGGCTAACTATTCTCAAGAC |
| | GTGAAGCAGTTCGCCAACGGATTTGTGGTGCGGATCGGCGCCGCTGCAAACAGCACCGGCACAGTGATTATTAGCCCTTCCACTAGCGCCACAATCCGGAAAATTTATCCTGCTTTCATGCT |
| | GGGAAGCAGCGTGGGAAATTTTAGCGATGGCAAGATGGGGCGGTTCTTCAACCACACCCTGGTGCTGCTGCCAGACGGCTGTGGGACCCTGCTGAGGGCTTTTTACTGCATCCTGGAGCCCC |
| | GGTCTGGAAATCATTGTCCTGCCGGCAATTCCTATACCTCTTTCGCAACCTATCACACTCCCGCTACCGACTGCTCTGATGGAAATTATAACAGAAACGCTTCTCTGAATAGCTTTAAGGAG |
| | TATTTCAATCTGCGCAACTGCACCTTTATGTACACTTATAACATCACTGAGGACGAAATCCTGGAATGGTTTGGCATCACTCAGACCGCCCAGGGCGTGCACCTCTTCTCTTCTAGGTACGT |
| | GGACCTGTACGGGGGGAACATGTTCCAGTTTGCTACCCTGCCTGTTTATGATACCATTAAGTACTATAGTATTATCCCACACTCAATTCGCTCCATTCAGTCTGATAGAAAGGCTTGGGCTG |
| | CCTTCTATGTGTACAAGCTGCAGCCCCTGACGTTTCTCCTGGATTTCAGCGTGGACGGGTATATCCGGCGCGCCATCGACTGCGGCTTTAACGACCTGTCCCAGCTGCACTGCTCTTACGAA |
| | TCCTTTGACGTGGAGTCAGGGGTCTACTCTGTGTCTTCTTTCGAAGCCAAACCCTCCGGGTCCGTGGTGGAGCAGGCCGAGGGCGTGGAGTGTGACTTCTCTCCACTGCTGAGCGGAACTCC |
| | ACCACAGGTCTACAACTTCAAACGGCTGGTGTTTACAAATTGTAATTATAACCTGACTAAGCTACTGAGCCTGTTCTCCGTGAACGACTTCACTTGTTCCCAGATCTCTCCCGCAGCTATTG |
| | CCAGTAATTGCTACTCTTCTCTGATCCTGGATTACTTCAGCTACCCTCTGTCTATGAAATCCGATCTGAGCGTGAGCTCCGCAGGACCAATCAGCCAGTTTAACTATAAGCAGAGCTTTTCT |
| | AACCCCACTTGCCTGATCCTGGCAACCGTGCCTCACAACCTGACCACTATTACCAAGCCACTGAAGTACAGCTACATCAATAAATGCAGCCGCTTTCTGAGCGACGACCGCACAGAGGTGCC |
| | CCAGCTCGTGAATGCCAACCAATACAGCCCTTGCGTCAGCATCGTGCCTTCCACAGTGTGGGAAGACGGAGACTACTATCGCAAGCAGCTGTCTCCCCTCGAGGGCGGCGGATGGCTGGTCG |
| | CTTCCGGCAGCACAGTGGCCATGACTGAGCAGCTCCAGATGGGGTTCGGGATCACCGTGCAGTATGGCACCGACACTAACTCCGTGTGCCCTAAACTGGAATTCGCAAACGACACCAAGATC |
| | GCAAGCCAGCTGGGAAATTGCGTGGAGTATTCCCTGTATGGCGTCAGCGGGAGGGGCGTGTTTCAGAATTGCACTGCCGTGGGAGTGAGGCAGCAGAGGTTTGTGTACGACGCCTATCAGAA |
| | CCTGGTGGGATATTATTCTGACGATGGCAATTATTATTGTCTGCGGGCTTGCGTCTCTGTGCCCGTGAGCGTGATTTACGACAAAGAAACCAAGACCCATGCTACACTGTTTGGCTCTGTGG |
| | CATGCGAGCACATCAGCTCCACTATGTCTCAGTATTCCCGCTCCACCAGATCTATGCTGAAGAGAAGAGACAGCACCTACGGCCCTCTGCAGACACCAGTGGGCTGCGTGCTGGGCCTGGTG |
| | AACTCCAGCCTGTTCGTGGAGGACTGCAAGCTGCCACTGGGCCAGTCCTTATGTGCACTGCCCGATACCCCATCTACTCTCACACCACGCAGCGTTCGGTCTGTGCCCGGGGAGATGAGACT |
| | GGCAAGCATCGCCTTCAACCACCCCATTCAGGTGGACCAGCTGAATTCCTCTTATTTTAAACTCTCCATCCCCACCAACTTCTCTTTTGGAGTGACACAGGAATATATCCAGACTACAATTC |
| | AGAAGGTGACAGTCGACTGCAAACAGTATGTTTGCAACGGGTTCCAGAAGTGCGAGCAGCTGCTGAGGGAGTACGGGCAGTTTTGCTCCAAGATCAATCAGGCCCTGCACGGCGCCAACCTG |
| | CGCCAGGATGACTCTGTCCGGAACCTCTTCGCCTCTGTGAAGTCTAGCCAGTCTAGCCCAATCATCCCTGGATTTGGCGGAGATTTTAATCTGACCCTGCTGGAACCTGTCAGCATCAGCAC |
| | AGGCAGCAGATCTGCCAGGTCCGCCATCGAGGACCTCCTGTTTGATAAGGTGACAATTGCAGACCCTGGATACATGCAGGGGTATGACGATTGTATGCAGCAGGGCCCTGCTTCCGCCAGGG |
| | ATCTCATTTGTGCACAGTATGTGGCCGGCTACAAGGTGCTGCCTCCTCTGATGGATGTCAACATGGAGGCTGCTTATACTTCTTCTCTGCTGGGCAGCATTGCTGGAGTGGGATGGACAGCA |
| | GGCCTGAGCAGCTTCGCCGCCATCCCTTTTGCCCAGTCTATTTTCTATAGGCTGAACGGAGTGGGGATCACTCAGCAGGTGCTGTCTGAAAACCAGAAGCTGATCGCTAACAAATTCAACCA |
| | GGCTCTGGGCGCAATGCAGACAGGGTTTACTACCACAAACGAGGCTTTTCACAAAGTGCAGGACGCCGTGAACAACAACGCCCAGGCTCTGAGCAAGCTGGCTTCTGAGCTGTCCAACACAT |
| | TTGGCGCTATCTCCGCCAGCATTGGCGACATTATCCAGAGACTCGACCCACCAGAACAGGATGCTCAGATTGACAGACTGATCAACGGACGCCTGACTACTCTGAACGCCTTCGTGGCACAG |
| | CAGCTGGTGCGCTCAGAGTCCGCTGCACTGTCTGCCCAGCTCGCTAAGGACAAGGTGAACGAGTGCGTGAAGGCTCAGTCTAAGCGGAGTGGATTTTGCGGACAGGGCACACACATCGTGAG |
| | CTTCGTGGTGAACGCTCCCAACGGCCTGTATTTTATGCATGTGGGCTACTACCCCTCTAACCATATCGAGGTCGTCTCCGCTTACGGCCTGTGTGACGCCGCTAACCCCACTAACTGCATTG |
| | CCCCCGTGAACGGCTATTTCATCAAGACCAACAATACTCGCATTGTGGACGAGTGGTCCTACACTGGCAGCAGCTTTTACGCCCCAGAACCAATCACATCCCTGAATACCAAGTATGTGGCA |
| | CCCCAGGTGACATACCAGAATATTTCTACTAATCTGCCTCCTCCCCTGCTGGGAAACTCCACCGGCATCGACTTTCAGGATGAGCTGGATGAGTTCTTCAAAAATGTGTCCACCAGTATTCC |
| | TAACTTTGGGTCCCTGACTCAGATCAATACCACTCTCCTGGACCTGACTTACGAGATGCTGAGCCTGCAGCAGGTGGTGAAGGCCCTGAACGAGTCCTATATCGATCTGAAGGAACTGGGCA |
| | ATTACACATATTATAATAAATGGCCTTGGTATATCTGGCTGGGATTCATTGCAGGCCTCGTGGCCCTGGCTCTGTGTGTCTTCTTTATTCTGTGTTGTACCGGATGTGGCACAAACTGCATG |
| | GGGAAGCTGAAGTGCAATCGGTGCTGTGACAGATATGAGGAGTACGACCTGGAGCCACACAAGGTTCACGTGTAA( SEQ ID NO: 573) |
| XBB.1.5_S2P | ATGTTCGTGTTTCTGGTGCTGCTGCCCCTGGTGAGCAGCCAGTGCGTGAACCTGATCACCAGAACACAGAGCTACACCAACAGCTTCACCAGAGGCGTGTACTACCCCGACAAGGTGTTCAG |
| | GAGCTCCGTGCTGCACAGCACCCAGGACCTGTTCCTGCCCTTCTTTTCCAACGTGACCTGGTTCCACGCCATCCACGTGTCCGGCACCAACGGCACCAAGCGCTTCGACAACCCCGCCCTGC |
| | CCTTCAACGACGGAGTGTACTTCGCTTCTACCGAGAAGTCCAACATCATCAGAGGCTGGATCTTTGGAACCACACTGGACAGCAAGACCCAGAGCCTGCTGATCGTGAATAACGCCACCAAC |
| | GTCGTGATCAAGGTGTGCGAGTTTCAGTTCTGCAACGACCCCTTCCTGGATGTGTACCAGAAGAATAACAAGAGCTGGATGGAGAGCGAGTTCAGGGTGTACAGCTCCGCCAACAATTGTAC |
| | CTTCGAGTACGTGTCCCAGCCTTTCCTGATGGACCTGGAGGGCAAGGAGGGCAACTTCAAAAACCTGAGGGAGTTCGTGTTCAAGAATATCGACGGCTACTTCAAGATCTACAGCAAACACA |
| | CCCCAATTAACCTGGAGAGGGACCTGCCTCAGGGCTTCTCCGCCCTGGAGCCCCTGGTGGACCTGCCCATCGGCATCAATATCACCAGATTCCAGACACTGCTGGCCCTGCACAGAAGCTAC |
| | CTGACCCCCGTGGACAGCAGCAGCGGCTGGACTGCCGGCGCCGCCGCTTACTACGTGGGCTACCTGCAGCCCAGGACCTTTCTGCTGAAGTACAACGAGAACGGCACCATCACCGACGCCGT |
| | GGATTGCGCCCTGGACCCCCTGTCCGAGACAAAGTGCACCCTGAAGTCTTTCACCGTGGAGAAGGGCATCTACCAGACCAGCAACTTCAGAGTGCAGCCCACCGAGTCCATCGTGCGCTTCC |
| | CCAACATCACCAACCTGTGCCCCTTCCACGAGGTGTTCAACGCCACAACCTTTGCCTCCGTGTACGCCTGGAACAGGAAGAGAATCAGCAACTGCGTGGCCGACTACAGCGTGATCTACAAT |
| | TTCGCCCCCTTCTTCGCCTTCAAGTGTTACGGCGTGAGCCCCACCAAGCTGAATGACCTGTGCTTCACAAACGTGTACGCCGACAGCTTCGTGATCAGAGGCAATGAGGTGAGCCAGATCGC |
| | CCCCGGCCAGACCGGCAACATTGCCGATTATAATTACAAGCTGCCCGACGACTTTACCGGCTGCGTGATCGCCTGGAATAGCAACAAGCTGGACTCTAAGCCCAGCGGCAACTATAATTACC |
| | TGTACAGACTGTTCAGAAAGAGCAAGCTGAAGCCTTTCGAGAGAGACATCAGCACAGAGATCTACCAGGCCGGCAACAAGCCATGCAATGGCGTGGCTGGCCCAAACTGCTACAGCCCCCTG |
| | CAGTCCTACGGCTTTCGGCCTACCTACGGCGTGGGCCACCAGCCCTACAGAGTGGTGGTGCTGAGCTTCGAGCTGCTGCACGCCCCCGCCACCGTGTGCGGCCCCAAAAAGAGCACCAACCT |
| | GGTGAAGAACAAGTGCGTGAACTTCAATTTCAACGGCCTGACCGGCACCGGCGTGCTGACCGAGTCCAACAAGAAGTTCCTGCCCTTTCAGCAGTTCGGCCGGGACATCGCCGACACCACCG |
| | ACGCCGTGAGGGACCCCCAGACCCTGGAGATCCTGGACATCACCCCATGTTCCTTTGGCGGCGTGAGCGTGATCACCCCTGGCACTAACACCAGCAATCAGGTGGCCGTGCTGTACCAGGGG |
| | GTGAACTGCACCGAGGTGCCCGTGGCTATCCATGCCGACCAGCTGACCCCCACCTGGAGGGTGTACAGCACAGGCAGCAACGTGTTCCAGACCAGGGCCGGCTGTCTGATCGGCGCCGAGTA |
| | CGTGAACAACTCTTACGAGTGCGACATCCCTATCGGCGCCGGCATCTGCGCCTCCTACCAGACCCAGACCAAGAGCCACAGAAGGGCCAGGTCCGTGGCCTCCCAGAGCATCATCGCCTACA |
| | CCATGTCCCTGGGCGCCGAAAACAGCGTGGCCTACAGCAACAACAGCATCGCAATCCCCACCAACTTCACCATCAGCGTGACCACCGAGATCCTGCCCGTGAGCATGACCAAGACCTCCGTG |
| | GACTGCACAATGTACATCTGCGGCGACAGCACTGAGTGCAGCAACCTGCTGCTGCAGTATGGCAGCTTCTGCACCCAGCTGAAAAGGGCCCTGACCGGCATTGCCGTGGAGCAGGACAAGAA |
| | CACCCAGGAGGTGTTCGCCCAGGTCAAGCAGATCTACAAGACCCCCCCCATCAAGTACTTTGGCGGCTTCAACTTTAGCCAGATCCTGCCCGACCCCAGCAAGCCCAGCAAGAGGTCCTTCA |
| | TCGAGGATCTGCTGTTCAACAAGGTGACACTGGCCGACGCCGGATTCATCAAGCAGTACGGCGACTGCCTGGGCGACATCGCCGCCAGGGACCTGATCTGCGCCCAGAAGTTTAACGGCCTG |
| | ACCGTGCTGCCTCCACTGCTGACCGATGAGATGATCGCCCAGTACACCAGCGCCCTGCTGGCCGGCACCATCACCTCTGGGTGGACCTTCGGCGCCGGCGCCGCCCTGCAGATCCCTTTCGC |
| | CATGCAGATGGCCTACAGATTCAATGGCATCGGAGTGACCCAGAACGTGCTGTACGAGAATCAGAAGCTGATCGCCAACCAGTTTAACTCCGCCATCGGCAAGATCCAGGACAGCCTGAGCA |
| | GCACCGCCAGCGCCCTGGGCAAGCTGCAGGATGTGGTGAACCACAACGCCCAGGCCCTGAACACCCTGGTGAAGCAGCTGTCCAGCAAGTTTGGCGCCATCTCCTCCGTGCTGAACGACATC |
| | CTGAGCCGGCTGGACCCCCCCGAGGCCGAGGTGCAGATCGACCGCCTGATCACTGGCCGGCTGCAGTCTCTGCAGACCTACGTGACCCAGCAGCTGATCAGAGCCGCCGAGATCAGAGCCAG |
| | CGCCAACCTGGCCGCCACCAAGATGAGCGAGTGTGTGCTGGGCCAGAGCAAGCGCGTGGACTTCTGTGGCAAGGGGTACCATCTGATGAGCTTCCCCCAGAGTGCCCCTCACGGCGTGGTGT |
| | TCCTGCACGTGACCTACGTGCCCGCTCAGGAAAAGAACTTCACAACCGCCCCCGCCATCTGCCACGACGGCAAGGCCCACTTCCCTCGCGAGGGCGTGTTCGTGAGCAACGGCACTCACTGG |
| | TTCGTGACCCAGCGGAACTTTTACGAGCCCCAGATCATCACCACCGACAATACCTTTGTGAGCGGCAACTGCGACGTGGTGATCGGCATCGTGAATAATACAGTGTACGACCCCCTGCAGCC |
| | TGAGCTGGACAGCTTCAAGGAGGAGCTGGACAAGTACTTTAAGAACCACACCAGCCCTGATGTGGATCTGGGCGACATCTCCGGCATCAACGCCAGCGTGGTGAACATCCAGAAGGAGATCG |
| | ATAGGCTGAATGAGGTGGCCAAGAACCTGAACGAGTCCCTGATCGACCTGCAGGAGCTGGGGAAGTACGAGCAGTACATCAAGTGGCCATGGTACATCTGGCTGGGCTTCATTGCTGGCCTG |
| | ATCGCCATCGTGATGGTGACCATCATGCTGTGCTGCATGACCAGCTGCTGCAGCTGCCTGAAGGGCTGCTGCTCTTGCGGCTCTTGCTGCAAGTTCGACGAGGACGACAGCGAGCCCGTGCT |
| | GAAGGGCGTGAAGCTGCACTACACCTAATAA (SEQ ID NO: 574) |
| Khosta2-WA1 | ATGGACTCTAAGGGGTCCAGCCAGAAAGGCTCCCGCCTGCTGCTGCTGCTGGTGGTGTCTAATCTCCTGCTGCCCCAGGGGGTGGTCGGCCAGAACATGACTAACATTTGCCCATTTGACCA |
| RRFN | GGTGTTTAACAAAACTCAGTTTCCCTCCGTGTACGCCTGGGAAAGGGTGAGAATCTCTGATTGTGTCAGCGACTATACAGTGCTGTATAACAGCTCCGCCAGCTTCTCCACTTTTAAGTGTT |
| | ACGGAGTGTCCCCCACCAAGCTGAACGACCTGTGCTTTAGCGGAGTGTATGCCGACTATTTTGTGGTGAAGGGCGACCACGTGCATCAGATCGCTCCCGGACAGACCGGAGTCATCGCCGAC |
| | TATAATTATAAACTGCCTTCAGAGTTCGTGGGATGCATCCTGGCCTGGAATACTAGAACAATTGACAGTAAAAGGGGATTTTACTATAGACTGTTCCGCCACGGGAATATTAGACCCTATGA |
| | GAGGGACACATCCAATGTCCCTTACAACGCTGCAGGCGGCACCTGCAACCAGCCCGGAACTCATAACTGCTATGAACCTCTGCAGGATTATGGCTTTACTTCCACCTCCGGCGTGGGGTATC |
| | AGCCCTTCCGGGTGGTGGTGCTGAGCTTTGAGAACAAGTCTGCCCCAGCCACCGTCTGCGGAGGCGGCTCCGGCGGGTCTGGGAATATCACAAACCTGTGCCCTTTCGGAGAGGTGTTCAAT |
| | GCAACCAGATTTGCTAGCGTGTACGCTTGGAACAGGAAGCGGATCTCCAACTGCGTGGCTGACTACAGCGTGCTGTATAACTCCGCTAGCTTTAGCACCTTCAAGTGTTATGGAGTGTCCCC |
| | TACAAAGCTGAATGATCTCTGCTTTACCAACGTGTACGCAGATTCCTTCGTGATTAGGGGAGATGAGGTGAGGCAGATTGCCCCCGGACAGACAGGAAAAATCGCCGACTACAACTATAAGC |
| | TGCCAGACGATTTTACAGGCTGTGTGATTGCCTGGAACAGCAACAATCTGGACAGCAAGGTGGGAGGAAATTACAACTACCTGAGACGCCTCTTCAGAAAGTCTAATCTGAAGCCCTTTGAG |
| | AGAGACATCTCCACCGAAATCTACCAGGCAGGGAGCACCCCATGCAATGGCGTCGAGGGCTTCAACTGCTACTTTCCACTGCAGAGCTACGGCTTTCAGCCCACAAACGGGGTGGGCTATCA |
| | GCCCTATAGGGTGGTGGTGCTGTCTTTTGAAAACAAGTCCGCACCTGCCACAGTGTGCGGCGGCGGCTCTGGAGGGAGTGGGGAGTCTCAGGTGCGCCAGCAGTTTTCCAAGGATATCGAGA |
| | AGCTCCTGAACGAGCAGGTGAACAAGGAAATGCAGAGCTCCAACCTGTATATGAGCATGTCCTCCTGGTGCTACACTCATAGCCTGGATGGCGCCGGGCTGTTCCTGTTTGATCACGCCGCC |
| | GAGGAATACGAGCACGCCAAGAAGCTGATTATCTTCCTGAACGAAAACAACGTCCCTGTGCAACTGACATCCATTTCTGCTCCCGAACACAAGTTCGAGGGCCTGACCCAGATCTTTCAGAA |
| | AGCATATGAACACGAGCAGCATATCTCTGAGTCCATCAACAACATCGTGGACCATGCAATCAAGAGCAAGGATCATGCTACTTTCAATTTCCTGCAGTGGTATGTGGCCGAGCAGCACGAAG |
| | AGGAGGTGCTGTTTAAGGACATCCTGGATAAGATCGAGCTGATTGGCAACGAAAATCACGGCCTGTATCTTGCAGACCAGTACGTGAAGGGAATTGCCAAGAGCCGCAAGAGCGGCAGCTAA |
| | (SEQ ID NO: 575) |
| WA1-Khosta2 | ATGGATAGCAAGGGAAGCTCCCAGAAGGGAAGCAGACTGCTGCTGCTGCTGGTGGTGAGCAACCTGCTGCTGCCCCAGGGCGTGGTGGGCCAAAATATCACAAACCTGTGCCCTTTCGGCGA |
| RRFN | GGTGTTCAATGCCACCAGGTTCGCCTCTGTGTACGCCTGGAATCGGAAGAGAATCAGCAATTGTGTGGCCGACTACTCCGTGCTGTACAATAGCGCCAGCTTCAGCACCTTCAAGTGCTACG |
| | GGGTGAGCCCCACAAAGCTGAATGACCTGTGCTTCACCAATGTGTACGCCGACAGCTTCGTGATCAGAGGCGACGAGGTGAGACAGATCGCCCCCGGCCAGACAGGCAAGATTGCCGACTAC |
| | AATTACAAACTGCCCGATGACTTCACAGGCTGCGTGATCGCCTGGAATAGCAACAATCTGGACAGCAAAGTGGGCGGCAATTACAACTACCTGCGGAGACTGTTTCGGAAGTCCAACCTGAA |
| | GCCCTTTGAGCGGGACATCAGCACCGAGATCTACCAGGCCGGCAGCACCCCTTGCAATGGCGTGGAGGGCTTCAACTGCTATTTCCCTCTGCAGAGCTACGGCTTCCAGCCCACCAATGGCG |
| | TGGGCTACCAGCCTTACAGAGTGGTGGTGCTGAGCTTCGAGAATAAGAGCGCTCCCGCCACAGTGTGTGGGGGGGGCTCCGGCGGCAGCGGCAATATGACAAATATCTGCCCCTTCGATCAG |
| | GTGTTCAACAAGACCCAGTTCCCCTCCGTGTACGCCTGGGAGCGCGTGAGAATCTCCGATTGCGTGAGCGACTACACCGTGCTGTACAATAGCTCCGCCTCTTTCTCTACATTCAAGTGTTA |
| | CGGCGTGAGCCCTACAAAGCTGAACGACCTGTGCTTCAGCGGCGTGTACGCCGACTACTTCGTGGTGAAGGGCGACCACGTGCACCAGATCGCCCCTGGCCAGACCGGAGTGATCGCCGATT |
| | ACAACTACAAGCTGCCCAGCGAATTCGTGGGCTGTATCCTGGCCTGGAATACCAGAACCATCGACAGCAAGAGAGGCTTCTATTACAGACTGTTCAGACACGGCAACATCCGGCCCTACGAA |
| | CGGGACACCTCCAATGTGCCTTACAATGCCGCCGGCGGCACCTGCAATCAGCCCGGAACCCACAACTGCTACGAACCTCTGCAGGACTACGGCTTCACCTCTACCAGCGGCGTGGGATACCA |
| | GCCCTTCCGGGTGGTGGTGCTGTCCTTTGAGAATAAGTCCGCCCCTGCCACCGTGTGTGGCGGAGGCAGCGGCGGCAGCGGAGAGAGCCAGGTGAGACAGCAGTTCTCTAAGGACATCGAGA |
| | AGCTGCTGAATGAGCAGGTGAATAAGGAGATGCAGAGCAGCAACCTGTACATGAGCATGAGCAGCTGGTGTTACACCCACTCCCTGGACGGCGCCGGCCTGTTCCTGTTCGATCATGCCGCC |
| | GAGGAGTACGAGCACGCTAAGAAGCTGATCATCTTCCTGAATGAAAATAATGTGCCAGTGCAGCTGACCAGCATCAGCGCTCCAGAGCACAAGTTCGAGGGCCTGACCCAGATCTTCCAGAA |
| | GGCCTACGAACACGAGCAGCACATCAGCGAGAGCATCAACAATATCGTGGACCACGCTATCAAGTCCAAGGACCACGCCACCTTCAATTTCCTGCAGTGGTACGTGGCCGAACAGCATGAGG |
| | AGGAAGTGCTGTTCAAGGATATCCTGGACAAGATCGAGCTGATCGGCAATGAGAATCACGGCCTGTACCTGGCCGACCAGTACGTGAAGGGCATTGCCAAGTCCAGAAAGAGCGGCTCCTAA |
| | (SEQ ID NO: 576) |
| SARS1-WA1 | ATGGACTCCAAGGGGAGCTCCCAGAAAGGGAGCAGGCTGCTGCTGCTCCTGGTGGTGAGCAACCTGCTGCTGCCACAGGGAGTAGTCGGACAGAATATCACCAACCTGTGCCCTTTCGGGGA |
| RRFN | GGTGTTTAATGCCACCAAGTTTCCTTCTGTGTACGCTTGGGAAAGGAAGAAAATCTCTAACTGTGTCGCTGATTATTCCGTGCTGTATAATTCTACCTTTTTCTCCACTTTTAAGTGCTATG |
| | GGGTGTCCGCTACCAAACTCAACGACCTGTGCTTTAGCAACGTGTACGCTGATAGCTTTGTGGTGAAAGGAGACGACGTGAGACAAATCGCCCCCGGCCAGACTGGGGTGATTGCAGATTAC |
| | AATTATAAACTGCCAGATGATTTTATGGGGTGTGTCCTGGCTTGGAACACACGCAACATTGACGCCACTAGCACCGGAAATTACAACTACAAACGGCGGTATCTGAGACACGGCAAGCTGCG |
| | CCCTTTTGAGAGGGACATTAGCAACGTGCCTTTCAGCCCAGACGGAAAGCCTTGCACACCCCCTGCACTGAATTGCTACTGGCCCCTGAACGACTACGGCTTCTATACCACCACTGGGATTG |
| | GGTATCAGCCATACCGGGTCGTGGTGCTGTCATTTGAGAATAAGTCCGCACCCGCCACTGTGTGCGGCGGAGGATCTGGAGGCTCCGGAAACATCACTAATCTCTGCCCCTTCGGAGAGGTG |
| | TTTAACGCCACAAGATTTGCTTCCGTGTACGCTTGGAACAGGAAGAGAATCTCCAATTGCGTGGCCGACTATTCTGTCCTGTACAACAGCGCCTCTTTCTCCACCTTCAAGTGCTATGGGGT |
| | TTCTCCTACAAAGCTGAACGACCTGTGTTTCACAAATGTTTACGCCGATTCCTTTGTGATCCGCGGCGATGAAGTGAGACAGATCGCTCCTGGGCAGACTGGCAAGATTGCCGATTATAACT |
| | ACAAGCTGCCTGATGACTTCACCGGATGTGTGATCGCTTGGAATTCCAATAACCTCGACTCTAAGGTGGGAGGCAATTACAACTATCTGCGGAGACTGTTCAGGAAATCTAATCTGAAGCCC |
| | TTTGAGCGCGATATCTCCACCGAGATCTACCAGGCCGGATCTACCCCTTGTAATGGAGTGGAAGGGTTTAATTGTTACTTCCCTCTGCAGTCCTATGGATTTCAGCCTACAAACGGCGTGGG |
| | CTACCAGCCTTACCGGGTCGTGGTGCTCAGCTTCGAGAACAAGAGCGCTCCAGCTACCGTTTGCGGAGGGGGGTCCGGCGGATCCGGAGAATCTCAGGTGCGCCAGCAGTTCTCCAAGGATA |
| | TCGAGAAGCTGCTGAACGAGCAGGTCAACAAAGAGATGCAGAGCTCCAATCTCTACATGAGCATGTCTAGCTGGTGTTACACACATTCCCTGGACGGCGCAGGCCTGTTTCTCTTCGACCAC |
| | GCTGCCGAGGAGTACGAGCATGCAAAAAAACTCATCATCTTTCTGAATGAGAACAACGTGCCCGTGCAGCTGACCTCTATCAGCGCACCAGAGCATAAGTTTGAAGGGCTGACACAGATTTT |
| | CCAGAAAGCCTACGAGCACGAGCAGCACATTTCTGAGTCTATCAACAACATTGTGGATCACGCTATCAAATCCAAGGATCACGCAACATTTAATTTTCTGCAGTGGTATGTTGCCGAGCAGC |
| | ACGAGGAGGAGGTGCTGTTCAAAGATATCCTGGACAAAATTGAGCTGATCGGCAATGAAAATCACGGGCTGTACCTGGCAGATCAGTATGTGAAAGGCATTGCTAAGAGCAGAAAGTCTGGA |
| | AGCTAA (SEQ ID NO: 577) |
| WA1-SARS1 | ATGGATTCTAAGGGATCCTCCCAGAAAGGGTCTAGGCTGCTGCTGCTTCTGGTTGTTTCCAACCTGCTGCTGCCTCAGGGCGTGGTGGGCCAGAATATCACCAACCTGTGTCCATTCGGCGA |
| RRFN | GGTGTTCAATGCAACCCGGTTTGCTAGTGTGTATGCTTGGAATCGCAAACGGATCTCCAACTGCGTGGCTGACTATAGTGTGCTCTATAACTCCGCCTCCTTCTCCACCTTTAAATGCTACG |
| | GCGTCTCTCCAACCAAACTGAACGACCTGTGTTTTACCAACGTGTATGCAGACTCTTTTGTGATCCGCGGGGATGAGGTGAGACAGATCGCACCCGGACAGACTGGAAAGATTGCCGATTAC |
| | AATTATAAGCTGCCTGACGATTTTACCGGGTGCGTGATCGCATGGAACTCCAACAACCTGGACTCTAAGGTGGGCGGGAATTATAACTACCTGCGCCGGCTGTTCAGGAAGTCCAATCTGAA |
| | GCCATTCGAACGGGATATTAGCACCGAGATCTATCAGGCCGGGTCCACCCCATGCAACGGCGTGGAGGGGTTCAACTGCTACTTCCCTCTCCAGAGCTATGGCTTCCAGCCCACAAATGGGG |
| | TGGGATATCAACCATACAGAGTGGTGGTCCTGAGCTTTGAGAACAAGAGCGCACCAGCAACCGTTTGTGGCGGCGGCAGCGGAGGGAGCGGCAACATCACCAACCTGTGTCCCTTTGGAGAA |
| | GTGTTCAACGCTACCAAATTCCCAAGCGTCTACGCCTGGGAGCGGAAGAAGATCTCTAACTGCGTGGCTGATTATTCTGTGCTGTACAACTCCACCTTCTTCTCCACATTTAAATGTTATGG |
| | AGTCTCCGCTACAAAGCTGAACGACCTGTGTTTCAGTAACGTGTACGCAGACTCATTTGTGGTCAAGGGAGATGACGTCCGGCAGATTGCCCCAGGCCAGACAGGAGTGATCGCAGACTACA |
| | ATTATAAGCTGCCAGACGATTTCATGGGCTGCGTGCTGGCTTGGAATACCAGAAACATCGATGCCACCTCTACTGGCAATTATAACTATAAGAGGAGGTATCTGAGACACGGCAAGCTGAGG |
| | CCCTTTGAGAGAGATATCAGCAACGTCCCATTTTCTCCTGACGGAAAGCCATGCACCCCACCTGCCCTGAACTGCTATTGGCCTCTGAATGATTATGGATTCTACACAACCACCGGAATCGG |
| | GTACCAGCCCTACAGGGTGGTGGTCCTCTCCTTCGAAAACAAGAGCGCTCCCGCTACAGTCTGCGGAGGGGGAAGCGGGGGATCTGGAGAGTCCCAGGTGCGCCAGCAGTTTAGCAAGGACA |
| | TCGAGAAGCTGCTGAACGAGCAAGTGAATAAAGAAATGCAGTCCAGCAACCTCTATATGTCCATGTCCTCCTGGTGCTACACCCATAGCCTGGATGGAGCTGGGCTGTTCCTGTTCGACCAC |
| | GCCGCCGAGGAGTATGAACATGCCAAAAAACTGATTATTTTTCTGAACGAAAATAACGTTCCTGTGCAGCTGACCTCTATCTCTGCCCCTGAACACAAGTTCGAAGGCCTGACCCAGATCTT |
| | TCAGAAGGCATACGAACATGAGCAGCACATCTCCGAGTCCATTAATAACATTGTGGATCACGCCATTAAGTCCAAAGACCATGCCACCTTCAATTTCCTGCAGTGGTACGTTGCCGAGCAGC |
| | ACGAAGAAGAGGTGCTGTTTAAGGACATCCTGGACAAAATTGAGCTGATTGGAAATGAGAACCACGGACTGTACCTCGCTGACCAGTACGTGAAGGGGATCGCAAAGTCTCGCAAGTCTGGA |
| | TCCTAA (SEQ ID NO: 578) |
| WA1- | ATGGATAGCAAGGGAAGCTCTCAGAAAGGGTCCAGACTGCTGCTGCTGCTGGTCGTTTCCAACCTGCTGCTCCCACAGGGCGTCGTGGGCCAAAACATCACTAACCTGTGCCCTTTCGGCGA |
| BANAL20247- | GGTGTTCAACGCCACAAGGTTCGCAAGCGTGTACGCTTGGAATAGAAAGCGCATCTCCAACTGCGTCGCAGATTACTCTGTGCTGTACAATAGCGCCAGCTTCTCCACATTCAAGTGTTACG |
| SARS1-WA1 RR- | GAGTGTCCCCAACCAAGCTGAACGACCTGTGCTTCACAAATGTGTACGCAGACAGCTTTGTGATCCGGGGAGATGAGGTGAGACAGATCGCCCCCGGACAGACTGGAAAAATCGCTGACTAC |
| mosSpFN | AACTACAAACTGCCCGATGATTTCACAGGATGCGTTATCGCCTGGAACAGCAACAACCTGGATTCCAAGGTTGGGGGCAACTACAACTACCTGCGCAGGCTGTTCAGGAAAAGCAACCTGAA |
| | ACCTTTCGAACGCGACATCTCTACAGAAATCTACCAGGCCGGATCCACCCCATGTAACGGAGTGGAAGGCTTTAACTGCTACTTCCCCCTGCAGTCTTACGGATTCCAGCCTACTAATGGCG |
| | TGGGCTATCAGCCTTACAGAGTTGTGGTGCTGTCCTTCGAGAATAAGTCTGCCCCTGCCACTGTGTGTGGGGGAGGGAGCGGCGGATCTGGGAATATTACTAACTTCTGCCCATTCGATAAG |
| | GTCTTCAACGCCACACGGTTTCCTAATGTCTACGCCTGGCAGAGGACCAAGATTTCTGACTGTATCGCTGACTATACTGTGCTGTACAACTCTACTAGCTTCAGCACTTTTAAATGCTACGG |
| | CGTGTCCCCAAGCAAGCTGATTGACCTGTGCTTTACATCCGTCTACGCCGACACATTTCTGATCCGGTTCAGCGAGGTCAGACAGATCGCTCCCGGCGAGACAGGCGTTATCGCTGACTACA |
| | ATTACAAACTGCCTGACGACTTCACAGGATGTGTGCTGGCTTGGAATACAGCCCAGCAGGATATCGGCAGCTATTTCTACAGATCCCACAGGGCAGTGAAACTGAAGCCTTTTGAGAGAGAT |
| | CTCAGCTCCGACGAAAACGGGGTGCGGACACTGTCTACCTATGACTTCAATCCTAACGTGCCTCTGGACTATCAGGCCACAAGAGTGGTGGTCCTGTCCTTCGAGAACAAATCCGCCCCAGC |
| | TACCGTTTGCGGCGGCGGATCCGGGGGCTCCGGATCTAGCCAGTGCGTGAACCTCACAACACGGACTCAGCTGCCCCCCGCCTACACTAACAGCTTCACACGGGGCGTCTACTATCCAGACA |
| | AAGTGTTCCGGAGCAGCGTGCTGCACAGCACTCAGGACCTCTTCCTGCCATTTTTCTCTAACGTGACATGGTTTCACGCAATTCACGTGTCTGGCACTAATGGGACTAAGCGGTTTGATAAC |
| | CCCGTCCTGCCTTTCAACGACGGCGTGTATTTCGCCTCCACTGAAAAAAGCAATATCATTAGAGGATGGATTTTCGGCACAACTCTGGATTCCAAAACCCAGTCCCTGCTCATCGTGAATAA |
| | TGCCACTAATGTGGTGATCAAGGTTTGTGAGTTCCAGTTCTGCAATGACCCTTTTCTGGGGGTGTACTATCACAAAAACAACAAGTCCTGGATGGAGAGCGAGTTTAGGGTTTACAGCAGCG |
| | ACCAACAATTGCACTTTCGAATACGTGTCTCAGCCCTTCCTCATGGACCTGGAGGGCAAACAGGGCAACTTTAAAAATCTGCGCGAGTTCGTGTTCAAGAACATCGACGGCTACTTCAAAAT |
| | TTACAGCAAGCACACACCTATCAACCTGGTGAGGGACCTGCCCCAGGGGTTCTCTGCCCTGGAGCCACTGGTCGACCTGCCAATTGGCATCAACATCACACGCTTCCAGACACTGCTGGCCC |
| | TGCATCGGAGCTACCTCACCCCTGGAGATAGCTCTAGCGGGTGGACTGCAGGAGCAGCAGCCTATTACGTGGGCTATCTGCAGCCCAGGACATTTCTCCTGAAGTATAACGAGAACGGAACT |
| | ATCACCGACGCAGTGGACTGCGCACTGGATCCACTGTCCGAAACAAAGTGCACCCTGAAGTCTTTCACCGTGGAGAAAGGAATTTACCAGACCTCCAATTTTCGCGTTCAGCCCACTGAGTC |
| | TATTGTCAGGTTTCCCATATTACCAACCTGTGCCCCTTCGGCGAGGTGTTTAACGCCACTAAATTCCCCAGCGTGTACGCATGGGAAAGGAAAAAGATCTCCAACTGCGTCGCCGATTACAG |
| | TGTGCTGTACAATAGCACCTTCTTCAGCACTTTCAAGTGCTATGGCGTCAGCGCCACCAAGCTGAATGACCTCTGCTTCAGCAATGTGTACGCCGACTCCTTTGTGGTGAAGGGAGATGACG |
| | TGCGGCAGATCGCCCCTGGCCAGACCGGAGTGATCGCCGATTATAATTATAAACTGCCTGACGATTTTATGGGCTGCGTGCTGGCATGGAATACACGGAACATCGACGCAACTTCCACAGGG |
| | AACTATAACTATAAGTACAGGTATCTCAGACACGGAAAGCTGCGCCCATTCGAGAGAGATATCTCTAACGTCCCTTTTTCCCCAGACGGCAAACCTTGCACCCCACCCGCTCTGAATTGCTA |
| | CTGGCCACTGAACGACTATGGCTTCTACACAACTACCGGCATTGGATACCAGCCATACCGCGTGGTGGTGCTGTCTTTTGAGCTCCTGAATGCTCCCGCCACAGTGTGCGGCCCAAAGAAGT |
| | CTACTAACCTCGTGAAAAAAAGTGTGTGAACTTCAACTTCAATGGCCTGACCGGAACTGGGGTGCTGACAGAGAGCAACAAGAAATTTCTGCCATTTCAGCAGTTCGGCAGAGACATCGCAG |
| | ACACTACCGATGCCGTGCGCGACCCCCAGACTCTGGAAATCCTCGACATCACCCCTTGCAGCTTCGGCGGCGTTTCTGTGATCACCCCCGGAACGAATACAAGCAACCAGGTGGCCGTGCTG |
| | TATCAGGACGTGAACTGCACAGAGGTGCCCGTGGCAATTCATGCCGACCAGCTCACTCCCACCTGGAGGGTCTACTCTACCGGCAGCAACGTGTTCCAGACCAGGGCCGGGTGTCTGATCGG |
| | AGCTGAGCATGTGAATAACAGTTACGAGTGTGACATCCCCATCGGGGCCGGAATTTGCGCTAGCTATCAGACCCAGACAAACTCTCCAGGAAGCGCATCCTCCGTGGCCTCTCAGAGCATTA |
| | TTGCCTACACCATGTCACTGGGAGCTGAAAACAGTGTCGCTTACTCCAATAACTCCATCGCCATCCCAACTAATTTCACAATTAGCGTGACCACCGAAATTCTGCCAGTGAGCATGACAAAG |
| | ACATCAGTGGATTGTACCATGTATATTTGCGGCGACTCCACCGAATGCAGCAATCTCCTGCTGCAGTACGGCTCCTTCTGCACTCAGCTGAACAGGGCCCTGACTGGCATTGCTGTCGAGCA |
| | GGACAAAAACACACAGGAAGTGTTCGCTCAGGTCAAGCAGATTTACAAGACTCCCCCAATTAAAGACTTCGGAGGCTTCAACTTTTCCCAGATTCTGCCAGACCCCTCCAAGCCATCCAAAC |
| | GCTCTTTCATCGAGGACCTGCTGTTTAACAAAGTGACACTGGCTGACGCTGGGTTCATTAAGCAGTATGGTGACTGCCTCGGGGATATCGCTGCCAGGGACCTGATTTGTGCTCAGAAATTC |
| | AACGGACTGACCGTGCTGCCTCCTCTGCTGACCGACGAAATGATCGCTCAGTACACCAGCGCACTGCTGGCAGGGACTATCACAAGTGGCTGGACATTCGGAGCCGGAGCCGCTCTGCAGAT |
| | CCCATTCGCCATGCAGATGGCATACAGATTCAACGGGATTGGAGTGACTCAGAACGTGCTGTATGAGAACCAGAAACTGATCGCTAACCAGTTCAACAGCGCTATTGGAAAAATTCAGGATT |
| | CTCTGTCTTCCACTGCTTCCGCCCTGGGGAAGCTGCAGGACGTGGTGAACCAGAACGCTCAGGCTCTCAATACACTGGTGAAGCAGCTGTCCTCCAACTTTGGGGCCATTTCTTCTGTGCTG |
| | AATGACATCCTCTCCAGACTGGATCCACCAGAGGCTGAGGTGCAGATCGATCGGCTGATCACTGGCAGGCTGCAGAGCCTCCAGACATATGTGACCCAGCAGCTGATTAGAGCTGCCGAAAT |
| | CAGGGCATCCGCTAATCTGGCAGCTACTAAGATGTCTGAGTGTGTGCTGGGCCAGTCCAAGCGGGTGGACTTTTGTGGAAAGGGCTATCACCTGATGTCTTTCCCTCAGTCCGCCCCACACG |
| | GGGTGGTGTTCCTGCACGTCACCTACGTCCCTGCCCAGGAGAAAAATTTTACCACCGCCCCAGCCATTTGCCACGATGGCAAAGCTCACTTCCCACGGGAAGGGGTCTTCGTGTCCAATGGG |
| | ACACACTGGTTCGTGACACAGAGGAATTTTTACGAACCACAGATCATCACCACCGATAACACCTTTGTGTCTGGAAATTGCGATGTGGTGATTGGAATTGTGAATAACACTGTGTATGATCC |
| | TCTGCAGTCCGAGCTGGATTCCATTAAAGAGGAGCTGGATAAGATTCATAAGAACGGGAGCGGCGGCAGCGGCGATATCATCAAACTGCTCAACGAGCAGGTGAACAAGGAGATGCAGAGCT |
| | CAAACCTGTACATGAGCATGTCTTCTTGGTGCTACACCCACAGCCTCGATGGAGCTGGACTGTTCCTGTTTGATCACGCCGCCGAGGAGTACGAACACGCAAAAAAGCTGATCATCTTCCTG |
| | AATGAGAATAACGTCCCTGTGCAGCTGACATCTATCTCTGCACCAGAGCATAAGTTTGAAGGACTGACACAGATTTTTCAGAAGGCATATGAGCATGAGCAGCATATCAGCGAGTCAATCAA |
| | CAACATCGTGGACCACGCAATCAAAAGCAAGGACCATGCTACCTTCAACTTCCTGCAGTGGTACGTGGCAGAGCAGCACGAAGAGGAGGTGCTGTTCAAGGACATTCTGGACAAGATTGAAC |
| | TGATCGGCAACGAAAACCACGGACTGTACCTGGCCGATCAGTATGTTAAGGGGATCGCCAAGTCTAGGAAAAGCGGATCTTAA (SEQ ID NO: 579) |
| SARS1-BANAL | ATGGACAGCAAGGGCTCCTCTCAGAAGGGATCTCGCCTGCTGCTCCTGCTCGTTGTGAGCAACCTGCTGCTCCCACAGGGGGTGGTCGGCCAGAACATTACCAACCTGTGCCCATTCGGCGA |
| 20247- | GGTCTTCAACGCTACAAAATTCCCCAGCGTGTACGCATGGGAAAGAAAAAAGATTAGCAATTGTGTCGCCGACTACTCCGTGCTGTACAATAGCACTTTCTTTTCCACTTTCAAGTGCTACG |
| Khosta2-WA1 | GGGTTTCCGCTACAAAGCTGAATGACCTGTGTTTTAGCAATGTGTATGCAGATTCTTTTGTGGTGAAGGGAGACGATGTCAGACAGATCGCCCCAGGCCAGACAGGCGTCATTGCCGATTAC |
| RR- | AATTATAAACTGCCAGACGACTTTATGGGATGCGTGCTGGCTTGGAACACCCGGAACATCGATGCAACCAGTACCGGCAACTATAATTATAAGCGCAGGTATCTGAGGCACGGCAAACTCAG |
| mosSpFN | ACCATTCGAGAGAGACATCTCTAACGTGCCTTTCTCTCCCGATGGTAAACCATGTACCCCCCCTGCCCTGAATTGTTATTGGCCACTGAACGATTATGGATTTTATACAACCACAGGCATCG |
| | GCTATCAGCCCTATAGAGTGGTGGTGCTGAGCTTCGAGAACAAATCCGCCCCAGCCACTGTCTGCGGAGGAGGAAGCGGCGGCAGCGGCAATATCACAAATTTTTGCCCCTTCGACAAGGTT |
| | TTTAACGCCACACGCTTCCCTAACGTGTACGCATGGCAGCGGACTAAGATTAGCGACTGTATTGCTGATTACACTGTCCTCTACAACTCTACCTCATTCTCCACCTTTAAATGCTATGGCGT |
| | ACAGCCCTTCTAAGCTGATCGACCTGTGCTTCACAAGCGTGTACGCAGACACCTTCCTGATCAGGTTCAGCGAGGTCAGACAGATTGCTCCCGGGGAGACTGGCGTCATTGCCGATTATAAC |
| | TATAAGCTGCCTGATGATTTCACTGGGTGCGTGCTGGCCTGGAACACCGCCCAGCAGGACATTGGAAGTTACTTTTATAGATCTCACAGGGCAGTCAAGCTGAAGCCATTCGAGAGGGACCT |
| | GTCTAGCGACGAAACGGCGTGAGAACCCTGAGCACTTATGATTTCAACCCTAATGTGCCTCTGGATTACCAGGCAACAAGAGTGGTCGTGCTGAGCTTCGAAAATAAGTCCGCCCCAGCCAC |
| | CTGTGTGCGGCGGGGGGTCTGGCGGAAGCGGATCCTCCCAGTGCGTTAACCTCACTACTCGGACCCAGCTGCCCCCTGCTTACACTAACTCCTTCACACGGGGCGTCTATTATCCTGATAAG |
| | GTCTTTAGAAGCAGCGTTCTGCACTCCACTCAGGACCTTTTCCTGCCCTTCTTCAGCAATGTGACTTGGTTTCACGCAATCCACGTCTCCGGGACCAACGGCACCAAGAGGTTCGACAATCC |
| | CGTGCTGCCATTTAACGACGGAGTTTATTTCGCTTCTACCGAGAAGTCTAACATTATCAGAGGCTGGATTTTTGGGACTACCCTGGACTCCAAGACACAGTCTCTGCTGATCGTCAATAAGC |
| | GCACTAACGTGGTCATTAAGGTGTGCGAGTTCCAATTTTGCAATGACCCCTTTCTGGGAGTGTACTATCACAAGAACAATAAGAGCTGGATGGAGTCTGAGTTCAGGGTGTATTCCTCTGCC |
| | AACAATTGCACCTTCGAGTATGTGTCACAGCCTTTTCTCATGGACCTGGAGGGCAAGCAGGGGAACTTCAAAAACCTGCGCGAATTCGTGTTCAAAAACATCGATGGATATTTTAAAATCTA |
| | CTCCAAGCATACCCCCATCAACCTGGTCAGGGACCTGCCCCAGGGCTTTTCTGCTCTGGAACCTCTGGTGGACCTGCCTATTGGGATCAACATCACAAGGTTTCAGACTCTGCTGGCACTGC |
| | ACCGCAGCTATCTGACCCCCGGAGATAGCAGCTCTGGGTGGACCGCCGGAGCTGCTGCTTATTATGTCGGATATCTGCAGCCACGGACATTTCTGCTGAAGTATAACAGAACGGAACCATTA |
| | CTGATGCTGTCGACTGCGCTCTGGACCCTCTGTCTGAAACCAAGTGCACCCTGAAGTCTTTCACAGTGGAGAAGGGCATTTATCAGACATCCAACTTTCGCGTGCAGCCCACAGAAAGCATC |
| | GTGAGATTCCCCAATATGACTAATATTTGTCCTTTCGACCAGGTCTTCAATAAGACCCAATTCCCATCCGTGTACGCCTGGGAACGCGTGAGAATTAGCGACTGTGTTAGCGACTATACCGT |
| | GCTGTATAATTCTTCAGCCTCTTTCAGCACATTCAAGTGTTACGGCGTGAGCCCTACCAAGCTGAACGACCTGTGCTTTTCTGGAGTGTACGCTGACTATTTTGTGGTGAAAGGGGATCACG |
| | TGCACCAGATTGCACCCGGGCAGACAGGGGTGATTGCAGATTATAATTACAAACTGCCATCTGAGTTTGTCGGATGTATCCTGGCCTGGAACACCAGAACAATCGACTCTAAAAGGGGCTTT |
| | TACTATCGGCTGTTTAGGCATGGGAATATCAGACCATATGAGAGAGATACAAGCAACGTTCCCTACAATGCTGCCGGGGGCACCTGCAATCAGCCAGGAACACACAACTGCTATGAACCCCT |
| | GCAGGACTATGGATTCACCAGCACTAGCGGCGTGGGCTACCAGCCTTTCCGCGTTGTGGTTCTCAGCTTCGAACTGCTGAACGCCCCAGCTACCGTTTGTGGACCTAAGAAGTCTACCAACC |
| | TGGTGAAGAACAAGTGTGTGAATTTTAACTTCAACGGACTGACTGGAACTGGCGTCCTGACTGAGTCCAATAAGAAGTTCCTGCCCTTTCAGCAGTTCGGGAGAGACATCGCCGATACAACC |
| | AGGATGCCGTGAGGGATCCACAAACCCTGGAGATCCTCGATATTACACCTTGCAGCTTCGGCGGGGTGTCAGTGATTACACCCGGCACTAATACTAGCAATCAGGTGGCTGTCCTGTACCAG |
| | GATGTGAATTGCACTGAGGTTCCAGTGGCAATCCACGCTGACCAGCTGACACCTACCTGGAGAGTGTATTCTACTGGCTCCAACGTGTTTCAGACACGGGCCGGCTGCCTGATCGGCCGAGC |
| | ATGTCAACAACTCATACGAATGTGACATCCCTATTGGGGCCGGGATCTGCGCCAGCTACCAGACTCAGACAAACAGCCCTGGGAGCGCATCTTCCGTGGCTAGCCAGTCTATTATCGCTTAC |
| | ACCATGTCCCTGGGAGCAGAAAACAGCGTGGCATATTCCAACAATTCTATCGCAATTCCAACTAACTTCACAATCAGCGTGACAACCGAAATCCTGCCCGTGTCCATGACAAAGACAAGCGT |
| | GGATTGCACCATGTATATCTGCGGAGATTCCACTGAATGCTCCAACCTGCTGCTGCAGTATGGATCCTTCTGCACACAGCTCAATAGGGCCCTCACAGGCATTGCTGTGGAACAAGATAAGA |
| | CACACCCAGGAGGTCTTCGCACAGGTGAAGCAGATTTATAAGACCCCCCCAATCAAAGACTTTGGAGGATTCAACTTTTCTCAGATTCTGCCCGACCCATCCAAGCCCAGCAAGCGTTCTTT |
| | CATCGAGGACCTCCTGTTTAACAAGGTCACACTGGCCGACGCTGGCTTCATCAAACAGTACGGGGACTGCCTCGGCGATATTGCCGCACGCGACCTTATCTGCGCCCAGAAATTTAACGGAC |
| | TGACTGTCCTGCCACCACTGCTCACCGACGAGATGATTGCCCAGTATACAAGCGCTCTGCTGGCAGGGACTATCACTAGCGGCTGGACATTCGGGGCCGGCGCTGCCCTGCAGATCCCCTTC |
| | GCCATGCAGATGGCCTATCGCTTTAACGGGATCGGCGTGACTCAGAATGTGTGTACGAGAACCAGAAGCTGATCGCTAACCAGTTCAACAGCGCTATTGGAAAAATCCAGGATTCCCTGAGC |
| | TCTACAGCTTCTGCACTGGGAAAACTGCAAGATGTGGTTAACCAGAATGCTCAGGCATTGAACACACTGGTCAAGCAGCTGTCTTCTAACTTTGGCGCCATTTCCAGCGTGCTGAATGATAT |
| | CCTGAGCAGACTGGACCCTCCAGAGGCAGAGGTGCAGATCGATCGGCTGATCACAGGGAGACTGCAGTCTCTGCAAACTTACGTGACTCAGCAACTGATTAGAGCTGCTGAGATTAGAGCCT |
| | CTGCCAACCTCGCCGCTACCAAGATGAGCGAGTGTGTCCTGGGACAGTCTAAGAGAGTGGATTTTTGCGGGAAGGGGTACCACCTGATGAGTTTCCCACAGTCCGCACCTCACGGAGTGGTG |
| | TTTCTGCACGTGACTTACGTGCCCGCCCAGGAGAAGAACTTCACAACAGCCCCTGCCATTTGCCACGACGGGAAGGCCCACTTTCCAAGAGAGGGGGTGTTCGTGTCCAACGGAACACATTG |
| | GTTTGTGACTCAGAGGAACTTCTACGAGCCTCAGATCATCACAACTGATAATACATTCGTTTCCGGCAATTGCGATGTCGTCATCGGCATCGTTAACAATACCGTGTATGACCCTCTGCAGT |
| | CTGAACTCGACAGTATCAAGGAGGAACTGGACAAGATCCACAAGAACGGCAGCGGCGGGTCTGGCGACATTATTAAGCTGCTGAATGAGCAGGTTAATAAGGAGATGCAGTCCAGCAACCTG |
| | TATATGTCTATGTCCTCCTGGTGCTACACTCATAGCCTGGACGGCGCCGGACTGTTCCTCTTCGACCATGCCGCAGAGGAATACGAGCACGCCAAGAAACTGATCATCTTCCTGAACGAAAA |
| | TAATGTGCCTGTGCAGCTGACTTCGATTAGCGCTCCCGAACATAAGTTTGAGGGGCTGACTCAGATCTTCCAGAAGGCCTACGAGCACGAGCAGCACATTAGCGAGTCTATTAACAACATTG |
| | TCGACCATGCCATTAAGAGCAAAGATCACGCCACTTTCAATTTCCTGCAATGGTATGTGGCAGAACAGCATGAAGAGGAGGTGCTGTTCAAGGATATCCTCGACAAGATTGAGCTGATTGG |
| | CAACGAGAATCATGGCCTGTATCTGGCTGATCAGTATGTGAAGGGCATCGCAAAGTCCCGGAAGTCAGGCTCCTAA (SEQ ID NO: 580) |
| Khosta2- | ATGGATTCCAAGGGATCCTCTCAGAAAGGAAGCCGGCTGCTGCTGCTGCTGGTTGTCTCTAACCTCCTCCTCCCTCAGGGCGTGGTGGGGCAGAATATGACAAACATCTGCCCTTTCGATCA |
| BANAL20247- | GGTGTTCAACAAAACTCAGTTTCCATCCGTGTATGCCTGGGAACGGGTCAGGATCTCTGATTGTGTGAGCGACTACACAGTGCTGTACAATAGCTCTGCCAGCTTTTCTACTTTCAAATGTT |
| SARS1 | ATGGGGTGTCCCCAACAAAGCTGAACGACCTGTGTTTTAGCGGCGTCTATGCCGACTACTTTGTGGTGAAAGGCGATCACGTGCACCAGATTGCCCCCGGGCAGACAGGAGTTATCGCCGAC |
| WA1 RR- | TATAACTATAAGCTGCCTTCCGAGTTCGTTGGATGTATTCTCGCCTGGAACACACGGACCATCGACAGCAAAAGAGGGTTTTACTATAGGCTGTTTAGGCATGGAAATATTAGGCCTTACGA |
| mosSpFN | GCGGGATACCTCTAACGTGCCTTATAACGCCGCTGGGGGAACCTGCAACCAGCCAGGCACACACAATTGTTACGAGCCTCTGCAGGACTACGGGTTCACATCTACCAGCGGCGTGGGGTATC |
| | AGCCTTTTAGGGTGGTCGTCCTGTCCTTTGAGAATAAGAGCGCCCCAGCCACTGTTTGTGGGGGGGGATCCGGAGGGAGCGGCAATATCACCAACTTCTGCCCATTCGATAAGGTTTTCAAC |
| | GCAACCCGGTTTCCAAACGTCTACGCCTGGCAGAGAACCAAAATTAGCGACTGCATCGCTGACTACACTGTGCTGTATAACTCCACATCCTTCTCCACATTTAAGTGCTACGGCGTGTCCCC |
| | ATCCAAACTCATCGACCTGTGCTTCACATCCGTGTACGCTGACACATTTCTGATCAGGTTTAGCGAGGTCAGACAGATCGCTCCAGGCGAAACAGGGGTTATCGCCGATTATAATTATAAAC |
| | TCCCAGATGACTTCACCGGATGTGTGCTCGCCTGGAACACCGCTCAGCAGGACATTGGCAGCTATTTCTACAGATCCCACCGGGCAGTCAAGCTGAAACCCTTTGAACGGGACCTGTCCAGC |
| | GATGAAAACGGCGTGCGCACCCTCAGCACTTATGATTTTAATCCAAATGTTCCCCTGGATTACCAGGCTACACGGGTCGTGGTGCTGTCATTCGAAAATAAGAGCGCCCCTGCAACCGTGTG |
| | CGGTGGCGGCTCCGGCGGGTCCGGGAGCTCTCAGTGCGTCAATCTGACTACACGGACTCAACTGCCTCCAGCATACACTAACAGCTTCACACGGGGCGTCTACTATCCTGACAAGGTGTTCA |
| | GGAGCAGCGTCCTGCACTCTACACAGGACCTCTTTCTGCCCTTCTTCAGCAATGTTACTTGGTTTCACGCCATTCACGTGAGCGGAACCAATGGCACTAAAAGGTTCGACAATCCTGTGCTG |
| | CCTTTCAACGACGGGGTTTACTTTGCCTCCACTGAGAAGTCCAATATCATTCGGGGGTGGATCTTTGGAACTACCCTGGACTCTAAGACTCAGTCTCTGCTGATCGTGAACAATGCAACTAA |
| | TGTGGTGATTAAGGTCTGCGAATTCCAGTTCTGCAATGATCCTTTTCTGGGCGTGTACTATCACAAGAATAACAAAAGCTGGATGGAGTCCGAATTTCGCGTTTACTCCTCTGCAAACAATT |
| | GCACTTTCGAGTACGTGAGCCAGCCATTTCTGATGGACCTCGAGGGAAAGCAGGGAAACTTCAAGAATCTGAGGGAATTTGTGTTCAAGAACATTGACGGCTATTTTAAGATCTACTCAAAA |
| | CATACTCCAATTAACCTCGTGAGAGATCTGCCACAGGGGTTCTCTGCACTGGAGCCTCTGGTCGACCTGCCTATTGGAATTAATATCACAAGATTCCAGACTCTGCTGGCCCTGCACCGCTC |
| | CTATCTGACACCAGGCGACAGCTCCAGCGGCTGGACCGCAGGCGCCGCCGCATATTATGTGGGCTATTTGCAGCCTAGAACTTTTCTCCTGAAGTACAACGAGAATGGCACAATTACAGATG |
| | CCGTCGACTGTGCACTGGACCCACTGTCTGAGACTAAGTGCACACTGAAATCCTTCACTGTGGAGAAAGGGATCTACCAGACTAGTAATTTTCGGGTGCAGCCAACAGAGAGCATCGTGCGG |
| | TTCCCTAACATTACTAACCTGTGCCCATTTGGGGAAGTGTTCAATGCAACAAAGTTCCCATCCGTGTACGCCTGGGAGAGAAAGAAAATTTCTAATTGCGTGGCAGACTACTCTGTGCTCTA |
| | CAACTCTACTTTCTTTTCCACCTTCAAGTGTTATGGCGTTAGCGCCACCAAACTGAACGACCTGTGCTTTTCTAACGTCTATGCCGATAGCTTTGTGGTCAAGGGCGATGACGTGAGGCAGA |
| | TCGCTCCTGGCCAAACAGGCGTGATTGCCGATTATAACTACAAGCTGCCTGACGACTTCATGGGATGCGTGCTGGCTTGGAATACTAGAAACATTGACGCAACCAGCACTGGAAATTACAAT |
| | TACAAGTACAGGTATCTGCGCCACGGCAAGCTGAGGCCCTTCGAAAGAGATATTTCAAACGTGCCCTTCTCTCCTGACGGAAAGCCATGTACCCCTCCAGCCCTGAACTGCTATTGGCCACT |
| | GAACGACTATGGGTTTTATACTACTACAGGCATTGGGTATCAGCCTTATAGGGTGGTTGTGCTGTCCTTTGAACTGCTGAACGCACCGGCTACCGTGTGTGGACCCAAAAAGTCCACAAACC |
| | TGGTGAAAAATAAGTGCGTGAACTTTAACTTCAACGGACTGACAGGGACTGGAGTGCTGACCGAGTCAAACAAAAAATTCCTGCCATTCCAGCAGTTCGGGAGGGACATCGCTGACACCACC |
| | GACGCTGTGAGGGATCCTCAGACCCTGGAAATTCTCGATATTACTCCCTGCTCCTTTGGAGGAGTCTCTGTCATTACACCTGGGACTAATACTAGCAATCAGGTGGCCGTGCTGTACCAGGA |
| | TGTTAATTGTACTGAAGTGCCTGTGGCCATCCACGCAGATCAGCTGACTCCCACCTGGAGAGTGTACAGTACAGGCTCTAACGTGTTCCAGACCAGGGCTGGCTGTCTGATCGGGGCCGAGC |
| | ACGTGAATAACAGCTATGAGTGTGACATTCCCATCGGGGCAGGAATCTGCGCCTCTTACCAGACACAGACCAACAGCCCTGGCTCAGCATCCAGCGTCGCCAGCCAGTCTATCATCGCCTAC |
| | ACAATGTCTCTGGGGGCAGAGAATAGCGTGGCTTATTCTAATAATTCTATTGCTATTCCCACAAACTTTACCATCAGCGTCACAACCGAGATCCTGCCAGTTTCCATGACAAAGACCTCCGT |
| | CGATTGTACCATGTACATCTGCGGCGACTCAACTGAATGTTCCAACCTGCTGCTGCAGTACGGAAGTTTTTGTACACAGCTGAATAGGGCCCTGACAGGCATCGCCGTTGAACAGGACAAGA |
| | ACACTCAGGAGGTGTTCGCCCAGGTCAAGCAGATCTACAAGACACCTCCTATCAAGGATTTCGGAGGATTCAACTTCTCCCAGATTCTGCCCGACCCTAGCAAGCCCAGTAAAAGGAGCTTC |
| | ATCGAGGACCTGCTATTCAACAAGGTCACACTCGCAGACGCCGGGTTCATTAAACAATACGGCGATTGCCTCGGCGACATTGCTGCACGCGACCTGATTTGTGCACAGAAGTTTAACGGCCT |
| | GACTGTCCTGCCCCCTCTGCTCACCGATGAAATGATCGCCCAGTACACCAGCGCTCTGCTGGCTGGGACTATTACATCCGGATGGACCTTTGGCGCAGGCGCTGCCCTGCAGATCCCATTCG |
| | CCATGCAGATGGCCTACAGATTCAATGGGATTGGGGTGACACAGAATGTGCTCTATGAGAATCAGAAACTGATAGCTAACCAGTTCAACAGCGCCATTGGGAAGATCCAGGACTCTCTGTCC |
| | TCTACTGCTTCCGCTCTGGGCAAACTGCAGGACGTCGTCAACCAGAACGCCCAAGCCCTGAACACCCTGGTGAAACAGCTGTCCAGCAATTTTGGCGCCATTTCCAGCGTCCTCAACGACAT |
| | CCTGAGCAGATTGGATCCACCCGAGGCCGAGGTTCAGATCGACAGACTGATCACCGGCCGACTGCAGAGCCTGCAGACTTACGTCACTCAGCAGCTGATCCGGGCAGCCGAGATCCGGGCCA |
| | GCGCGAATCTGGCAGCTACAAAGATGTCCGAGTGCGTGCTGGGCCAATCCAAGAGAGTGGACTTCTGCGGAAAGGGCTATCACCTGATGAGCTTTCCTCAGTCTGCCCCTCACGGCGTGGTT |
| | TTCCTGCATGTGACATACGTGCCAGCACAAGAGAAGAACTTCACAACCGCTCCTGCTATCTGCCACGATGGGAAAGCACACTTTCCTCGCGAGGGCGTGTTTGTGTCCAATGGGACACACTG |
| | GTTTGTGACCCAGAGAAATTTTTATGAGCCACAGATCATTACTACCGACAATACATTCGTGTCCGGAAACTGTGACGTGGTGATTGGGATTGTGAACAATACTGTGTATGACCCACTCCAGA |
| | GCGAGCTGGATTCTATCAAAGAAGAACTGGACAAAATCCATAAGAACGGCAGCGGAGGCTCCGGAGATATTATTAAGCTGCTGAACGAACAGGTTAATAAGGAGATGCAGAGCAGCAACCTG |
| | TATATGAGTATGTCCTCTTGGTGCTACACTCACTCCCTGGACGGGGCCGGGCTGTTCCTGTTCGACCACGCCGCCGAGGAGTATGAGCATGCTAAGAAGCTGATTATTTTTCTGAACGAAAA |
| | CAACGTGCCTGTGCAACTGACAAGCATTTCCGCCCCTGAGCATAAATTTGAGGGACTCACACAGATTTTCCAGAAAGCCTACGAACATGAGCAGCACATCAGCGAGTCTATCAACAACATTG |
| | TGGACCACGCTATCAAGTCCAAGGACCACGCCACCTTCAACTTCCTGCAGTGGTATGTGGCTGAGCAGCACGAGGAGGAAGTGCTGTTTAAGGACATCCTGGACAAGATTGAACTAATTGGA |
| | AACGAAAATCATGGGCTGTACCTGGCTGACCAGTATGTTAAAGGAATCGCTAAGTCCCGGAAGTCCGGGTCCTAA (SEQ ID NO: 581) |
| XBB.1.5 SpFN | ATGGACTCCAAGGGCTCCAGCCAGAAGGGCAGCCGCCTGCTGCTGCTGCTGGTGGTGTCAAACCTGCTGCTGCCACAGGGGGTGGTGGGCAGCAGCCAGTGCGTGAACCTGATCACCAGAAC |
| | ACAGAGCTACACCAACAGCTTCACCAGAGGCGTGTACTACCCCGACAAGGTGTTCAGGAGCTCCGTGCTGCACAGCACCCAGGACCTGTTCCTGCCCTTCTTTTCCAACGTGACCTGGTTCC |
| | ACGCCATCCACGTGTCCGGCACCAACGGCACCAAGCGCTTCGACAACCCCGCCCTGCCCTTCAACGACGGAGTGTACTTCGCTTCTACCGAGAAGTCCAACATCATCAGAGGCTGGATCTTT |
| | GGAACCACACTGGACAGCAAGACCCAGAGCCTGCTGATCGTGAATAACGCCACCAACGTCGTGATCAAGGTGTGCGAGTTTCAGTTCTGCAACGACCCCTTCCTGGATGTGTACCAGAAGAA |
| | TAACAAGAGCTGGATGGAGAGCGAGTTCAGGGTGTACAGCTCCGCCAACAATTGTACCTTCGAGTACGTGTCCCAGCCTTTCCTGATGGACCTGGAGGGCAAGGAGGGCAACTTCAAAAACC |
| | TGAGGGAGTTCGTGTTCAAGAATATCGACGGCTACTTCAAGATCTACAGCAAACACACCCCAATTAACCTGGAGAGGGACCTGCCTCAGGGCTTCTCCGCCCTGGAGCCCCTGGTGGACCTG |
| | CCCATCGGCATCAATATCACCAGATTCCAGACACTGCTGGCCCTGCACAGAAGCTACCTGACCCCCGTGGACAGCAGCAGCGGCTGGACTGCCGGCGCCGCCGCTTACTACGTGGGCTACCT |
| | GCAGCCCAGGACCTTTCTGCTGAAGTACAACGAGAACGGCACCATCACCGACGCCGTGGATTGCGCCCTGGACCCCCTGTCCGAGACAAAGTGCACCCTGAAGTCTTTCACCGTGGAGAAGG |
| | GCATCTACCAGACCAGCAACTTCAGAGTGCAGCCCACCGAGTCCATCGTGCGCTTCCCCAACATCACCAACCTGTGCCCCTTCCACGAGGTGTTCAACGCCACAACCTTTGCCTCCGTGTAC |
| | GCCTGGAACAGGAAGAGAATCAGCAACTGCGTGGCCGACTACAGCGTGATCTACAATTTCGCCCCCTTCTTCGCCTTCAAGTGTTACGGCGTGAGCCCCACCAAGCTGAATGACCTGTGCTT |
| | CACAAACGTGTACGCCGACAGCTTCGTGATCAGAGGCAATGAGGTGAGCCAGATCGCCCCCGGCCAGACCGGCAACATTGCCGATTATAATTACAAGCTGCCCGACGACTTTACCGGCTGCG |
| | TGATCGCCTGGAATAGCAACAAGCTGGACTCTAAGCCCAGCGGCAACTATAATTACCTGTACAGACTGTTCAGAAAGAGCAAGCTGAAGCCTTTCGAGAGAGACATCAGCACAGAGATCTAC |
| | CCAGGCCGGCAACAAGCCATGCAATGGCGTGGCTGGCCCAAACTGCTACAGCCCCCTGCAGTCCTACGGCTTTCGGCCTACCTACGGCGTGGGCCACCAGCCCTACAGAGTGGTGGTGCTGA |
| | GCTTCGAGCTGCTGCACGCCCCCGCCACCGTGTGCGGCCCCAAAAAGAGCACCAACCTGGTGAAGAACAAGTGCGTGAACTTCAATTTCAACGGCCTGACCGGCACCGGCGTGCTGACCGAG |
| | TCCAACAAGAAGTTCCTGCCCTTTCAGCAGTTCGGCCGGGACATCGCCGACACCACCGACGCCGTGAGGGACCCCCAGACCCTGGAGATCCTGGACATCACCCCATGTTCCTTTGGCGGCGT |
| | GAGCGTGATCACCCCTGGCACTAACACCAGCAATCAGGTGGCCGTGCTGTACAGGGGGTGAACTGCACCGAGGTGCCCGTGGCTATCCATGCCGACCAGCTGACCCCCACCTGGAGGGTGTA |
| | CAGCACAGGCAGCAACGTGTTCCAGACCAGGGCCGGCTGTCTGATCGGCGCCGAGTACGTGAACAACTCTTACGAGTGCGACATCCCTATCGGCGCCGGCATCTGCGCCTCCTACCAGACCC |
| | AGACCAAGAGCCCAGGATCAGCCTCATCCGTGGCCTCCCAGAGCATCATCGCCTACACCATGTCCCTGGGCGCCGAAAACAGCGTGGCCTACAGCAACAACAGCATCGCAATCCCCACCAAC |
| | TTCACCATCAGCGTGACCACCGAGATCCTGCCCGTGAGCATGACCAAGACCTCCGTGGACTGCACAATGTACATCTGCGGCGACAGCACTGAGTGCAGCAACCTGCTGCTGCAGTATGGCAG |
| | CTTCTGCACCCAGCTGAAAAGGGCCCTGACCGGCATTGCCGTGGAGCAGGACAAGAACACCCAGGAGGTGTTCGCCCAGGTCAAGCAGATCTACAAGACCCCCCCCATCAAGTACTTTGGCG |
| | GCTTCAACTTTAGCCAGATCCTGCCCGACCCCAGCAAGCCCAGCAAGAGGTCCTTCATCGAGGATCTGCTGTTCAACAAGGTGACACTGGCCGACGCCGGATCATCAAGCAGTACGGCGACT |
| | GCCTGGGCGACATCGCCGCCAGGGACCTGATCTGCGCCCAGAAGTTTAACGGCCTGACCGTGCTGCCTCCACTGCTGACCGATGAGATGATCGCCCAGTACACCAGCGCCCTGCTGGCCGGC |
| | ACCATCACCTCTGGGTGGACCTTCGGCGCCGGCGCCGCCCTGCAGATCCCTTTCGCCATGCAGATGGCCTACAGATTCAATGGCATCGGAGTGACCCAGAACGTGCTGTACGAGAATCAGAA |
| | GCTGATCGCCAACCAGTTTAACTCCGCCATCGGCAAGATCCAGGACTGGGCAAGCTGCAGGATGTGGTGAACCACAACGCCCAGGCCCTGAACACCCTGGTGAAGCAGCTGTCCAGCAAGTT |
| | TGGCGCCATCTCCTCCGTGCTGAACGACATCCTGAGCCGGCTGGACCCCCCCGAGGCCGAGGTGCAGATCGACCGCCTGATCACTGGCCGGCTGCAGTCTCTGCAGACCTACGTGACCCAGC |
| | AGCTGATCAGAGCCGCCGAGATCAGAGCCAGCGCCAACCTGGCCGCCACCAAGATGAGCGAGTGTGTGCTGGGCCAGAGCAAGCGCGTGGACTTCTGTGGCAAGGGGTACCATCTGATGAGC |
| | TTCCCCCAGAGTGCCCCTCACGGCGTGGTGTTCCTGCACGTGACCTACGTGCCCGCTCAGGAAAAGAACTTCACAACCGCCCCCGCCATCTGCCACGACGGCAAGGCCCACTTCCCTCGCGA |
| | GGGCGTGTTCGTGAGCAACGGCACTCACTGGTTCGTGACCCAGCGGAACTTTTACGAGCCCCAGATCATCACCACCGACAATACCTTTGTGAGCGGCAACTGCGACGTGGTGATCGGCATCG |
| | TGAATAATACAGTGTACGACCCCCTGCAGTCAGAGCTGGACAGCATCAAGGAGGAGCTGGACAAGATCCACAAGAACGGCTCCGGCGGCAGCGGCGACATCATCAAGCTGCTGAACGAACAG |
| | GTGAATAAGGAGATGCAGAGCAGCAACCTGTACATGAGCATGTCCTCCTGGTGCTACACCCATAGCCTGGACGGCGCCGGCCTGTTCCTGTTCGACCACGCCGCCGAGGAGTACGAGCACGC |
| | CAAGAAGCTGATCATCTTCCTGAACGAGAACAATGTGCCTGTGCAGCTGACTTCCATCAGCGCCCCCGAGCACAAGTTCGAGGGCCTGACCCAGATCTTCCAGAAGGCCTACGAGCATGAGC |
| | AGCACATCAGCGAGAGCATCAACAACATCGTGGATCACGCCATCAAGAGCAAGGATCACGCCACTTTCAACTTCCTGCAGTGGTACGTGGCCGAGCAGCACGAGGAGGAGGTGCTGTTCAAG |
| | GACATCCTGGACAAGATCGAGCTGATCGGCAATGAGAACCACGGCCTGTACCTGGCCGACCAGTACGTCAAGGGCATCGCCAAGAGCAGAAAGAGCGGCTCCTAATAA |
| | (SEQ ID NO: 582) |

What is claimed is:

1. An immunogenic composition comprising at least two antigenic coronavirus peptides comprising at least a first antigenic coronavirus peptide and a second antigenic coronavirus peptide, or one or more messenger RNA (mRNA) molecules encoding them, wherein each antigenic coronavirus peptide is independently selected from:
a. a receptor-binding domain (RBD or R) of a coronavirus, or a fragment or variant thereof,
b. an N-terminal domain (NTD) of a coronavirus, or a fragment or variant thereof,
c. an S1 domain of a coronavirus, or a fragment or variant thereof,
d. a stabilized spike S-2P domain of a coronavirus, or a fragment or variant thereof,
e. a stabilized spike S domain of a coronavirus, or a fragment or variant thereof, and
f. a stabilized spike S-trimer of a coronavirus, or a fragment or variant thereof,
g. wherein the antigenic coronavirus peptides include antigenic coronavirus peptides selected from the following combinations of strains:
(i) two or more selected clade 1b;
(ii) one or more selected from clade 1b and one or more selected from clade 1aSARS-CoV-1;
(iii) one or more selected from clade 1b, and one or more selected from clade 1a and one or more selected from clade 2;
(iv) one or more selected from clade 1b, and one or more selected from clade 1a, and one or more selected from clade 3;
(v) one or more selected from clade 1b, one or more selected from clade 2, and one or more selected from clade 3; and
(v) one or more selected from clade 1a, one or more selected from clade 2, and one or more selected from clade 3.

2. The composition of claim 1, wherein at least one of said antigenic coronavirus peptides is an S-2P peptide, optionally wherein the S-2P peptide comprises the amino acid sequence of any one of SEQ ID NOs 536-543, or a sequence having at least 80%, at least 85%, at least 90%, at least 95%, at least 96%, at least 97%, at least 98%, or at least 99% homology or identity thereto.

3. The composition of claim 1 or claim 2, wherein at least one of said antigenic coronavirus peptides is comprised in a fusion protein comprising the antigenic coronavirus peptide and a nanoparticle-forming protein, wherein two or more of said antigenic coronavirus peptides may be comprised in the same or different fusion proteins with a nanoparticle-forming protein.

4. An immunogenic composition comprising a nanoparticle comprising at least two antigenic coronavirus peptides comprising at least a first antigenic coronavirus peptide and a second antigenic coronavirus peptide, or one or more messenger RNA (mRNA) molecules encoding the at least two antigenic coronavirus peptides,
wherein each antigenic coronavirus peptide is independently selected from:
a. a receptor-binding domain (RBD or R) of a coronavirus, or a fragment or variant thereof,
b. an N-terminal domain (NTD) of a coronavirus, or a fragment or variant thereof,
c. an S1 domain of a coronavirus, or a fragment or variant thereof,
d. a stabilized extracellular spike S-2P domain of a coronavirus, or a fragment or variant thereof,
e. a stabilized extracellular spike S domain of a coronavirus, or a fragment or variant thereof, and
f. a stabilized extracellular spike S-trimer of a coronavirus, or a fragment or variant thereof,
wherein each antigenic coronavirus peptide is comprised in a fusion protein comprising the antigenic coronavirus peptide and a nanoparticle-forming protein, wherein the antigenic coronavirus peptides may be comprised in the same or different fusion proteins,
wherein the composition comprises antigenic coronavirus peptides from at least two different coronavirus strains, or one or more mRNA molecules encoding them.

5. The composition of claim 4, wherein each antigenic coronavirus peptide is from a coronavirus strain independently selected from clade 1a, clade 1b, clade 2, lade 3, and Middle East respiratory syndrome-related coronavirus (MERS-CoV), optionally wherein at least the first and second antigenic coronavirus peptides are from coronavirus strains of different clades.

6. The composition of claim 4 or claim 5, wherein at least the first and second antigenic coronavirus peptides are from different coronavirus strains independently selected from WA-1, Beta, Omicron BQ.1.1, Omicron XBB.1.5, a strain of SARS-CoV-1, BANAL20-247, Khosta2, and MERS-CoV.

7. The composition of any one of the preceding claims, wherein the antigenic coronavirus peptides include antigenic coronavirus peptides selected from the following combinations of strains:
(i) two or more selected from WA-1, Beta, Omicron XBB.1.5, and Omicron BQ1.1;
(ii) one or more selected from WA-1, Beta, Omicron XBB.1.5, and Omicron BQ1.1, and strains of SARS-CoV-1;
(iii) one or more selected from WA-1, Beta, Omicron XBB.1.5, and Omicron BQ1.1, and one or more selected from strains of SARS-CoV-1, BANAL20-247, and Khosta-2;
(iv) one or more selected from WA-1, Beta, Omicron XBB.1.5, and Omicron BQ1.1, and one or more selected from strains of SARS-CoV-1, BANAL20-247, and Khosta-2, and MERS-CoV; and
(v) two or more selected from strains of SARS-CoV-1, BANAL20-247, and Khosta-2.

8. The composition of any one of the preceding claims, wherein the antigenic coronavirus peptides include antigenic coronavirus peptides selected from the following combinations of strains:
a. WA-1, Beta, and Omicron BQ1.1;
b. WA-1, Omicron BQ.1.1, and SARS-CoV-1;
c. WA-1, SARS-CoV-1, and Khosta2;
d. WA-1, SARS-CoV-1, and BANAL20-247;
e. WA-1, SARS-CoV-1, and MERS-CoV; and
f. SAR-CoV-1, Khosta-2, and BANAL20-247.

9. The composition of any one of the preceding claims, wherein the antigenic coronavirus peptides include antigenic coronavirus peptides selected from the following combinations of strains:
(i) WA-1, Beta, and Omicron BQ.1.1 (or XBB.1.5), optionally wherein the antigenic coronavirus peptides are RBD antigens comprised in RFN fusion proteins R(WA-1)FN, R(Beta)FN, and R(BQ1.1)FN, or are Spike antigens comprised in S-2P (e.g., S(WA-1)-2P, S(Beta)-2P, and S(BQ1.1)-2P) or SpFN fusion proteins Sp(WA-1)FN, Sp(Beta)FN, and Sp(BQ1.1)FN, or are mosaic antigens comprised in RmosSpFN or RRmosSpFN fusion proteins, or any combination thereof;
(ii) WA-1, Omicron BQ.1.1 (or XBB1.5), and SARS-CoV-1, optionally wherein the antigenic coronavirus peptides are RBD antigens comprised in RFN fusion proteins R(WA-1)FN, R(BQ 1.1)FN, and R(SARS-CoV-1)FN, or are Spike antigens comprised in S-2P (e.g., S(WA-1)-2P, S(BQ 1.1)-2P, and S(SARS-CoV-1)-2P) or SpFN fusion proteins Sp(WA-1)FN, Sp(BQ 1.1)FN, and Sp(SARS-CoV-1)FN, or are mosaic antigens comprised in RmosSpFN or RRmosSpFN fusion proteins, or any combination thereof;
(iii) WA-1, SARS-CoV-1, and Khosta2, optionally wherein the antigenic coronavirus peptides are RBD antigens comprised in RFN fusion proteins R(WA-1)FN, R(SARS-CoV-1)FN, and R(Khosta2)FN, or are Spike antigens comprised in S-2P (e.g., S(WA-1)-2P, S(SARS-CoV-1)-2P, and S(Khosta2)-2P) or SpFN fusion proteins Sp(WA-1)FN, Sp(SARS-CoV-1)FN, and Sp(Khosta2)FN, or are mosaic antigens comprised in RmosSpFN or RRmosSpFN fusion proteins, or any combination thereof;
(iv) WA-1, SARS-CoV-1, and BANAL20-247, optionally wherein the antigenic coronavirus peptides are RBD antigens comprised in RFN fusion proteins R(WA-1)FN, R(SARS-CoV-1)FN, and R(BANAL20-247)FN, or are Spike antigens comprised in S-2P (e.g., S(WA-1)-2P, S(SARS-CoV-1)-2P, and S(BANAL20-247)-2P) or SpFN fusion proteins Sp(WA-1)FN, Sp(SARS-CoV-1)FN, and Sp(BANAL20-247)FN, or are mosaic antigens comprised in RmosSpFN or RRmosSpFN fusion proteins, or any combination thereof;
(v) WA-1, SARS-CoV-1, and MERS-CoV, optionally wherein the antigenic coronavirus peptides are RBD antigens comprised in RFN fusion proteins R(WA-1)FN, R(SARS-CoV-1)FN, and R(MERS-CoV)FN, or are Spike antigens comprised in S-2P (e.g., S(WA-1)-2P, S(SARS-CoV-1)-2P, and S(MERS-CoV)-2P) or SpFN fusion proteins Sp(WA-1)FN, Sp(SARS-CoV-1)FN, and Sp(MERS-CoV)FN, or are mosaic antigens comprised in RmosSpFN or RRmosSpFN fusion proteins, or any combination thereof;
(vi) SAR-CoV-1, Khosta-2, and BANAL20-247, optionally wherein the antigenic coronavirus peptides are RBD antigens comprised in RFN fusion proteins R(SARS-CoV-1)FN, R(Khosta2)FN, and R(BANAL20-247)FN, or are Spike antigens comprised in S-2P (e.g., S(SARS-CoV-1)-2P, S(Khosta2)-2P, and S(BANAL20-247)-2P) or SpFN fusion proteins Sp(SARS-CoV-1)FN, Sp(Khosta2)FN, and Sp(BANAL20-247)FN, or are mosaic antigens comprised in RmosSpFN or RRmosSpFN fusion proteins, or any combination thereof;
(vii) Beta, Omicron BQ.1.1 (or XBB1.5), and SARS-CoV-1, optionally wherein the antigenic coronavirus peptides are RBD antigens comprised in RFN fusion proteins R(Beta)FN, R(BQ 1.1)FN, and R(SARS-CoV-1)FN, or are Spike antigens comprised in S-2P (e.g., S(Beta)-2P, S(BQ 1.1)-2P, and S(SARS-CoV-1)-2P) or SpFN fusion proteins Sp(Beta)FN, Sp(BQ 1.1)FN, and Sp(SARS-CoV-1)FN, or are mosaic antigens comprised in RmosSpFN or RRmosSpFN fusion proteins, or any combination thereof;
(viii) Beta, Omicron XBB.1.5, and SARS-CoV-1, optionally wherein the antigenic coronavirus peptides are RBD antigens comprised in RFN fusion proteins R(Beta)FN, R(XBB 1.5)FN, and R(SARS-CoV-1)FN, or are Spike antigens comprised in S-2P (e.g., S(Beta)-2P, S(XBB 1.5)-2P, and S(SARS-CoV-1)-2P) or SpFN fusion proteins Sp(Beta)FN, Sp(XBB1.5)FN, and Sp(SARS-CoV-1)FN, or are mosaic antigens comprised in RmosSpFN or RRmosSpFN fusion proteins, or any combination thereof;
(ix) Omicron BQ.1.1, SARS-CoV-1, and Khosta2, optionally wherein the antigenic coronavirus peptides are RBD antigens comprised in RFN fusion proteins R(Omicron BQ.1.1)FN, R(SARS-CoV-1)FN, and R(Khosta2)FN, or are Spike antigens comprised in S-2P (e.g., S(Omicron BQ.1.1)-2P, S(SARS-CoV-1)-2P, and S(Khosta2)-2P) or SpFN fusion proteins Sp(Omicron BQ.1.1)FN, Sp(SARS-CoV-1)FN, and Sp(Khosta2)FN, or are mosaic antigens comprised in RmosSpFN or RRmosSpFN fusion proteins, or any combination thereof.

10. The composition of any one of claims 3-9, wherein the nanoparticle-forming peptide comprises or is a ferritin protein or a fragment or variant thereof.

11. The composition of any one of claims 3-9, wherein the nanoparticle-forming peptide comprises or is Helicobacter pylori ferritin (Hpf) or a fragment or variant thereof.

12. The composition of any one of claims 3-9, wherein the nanoparticle-forming peptide comprises an amino acid sequence selected from:
a. ESQVRQQFSKDIEKLLNEQVNKEMQSSNLYMSMSSWCYTHSLDGAGLFL FDHAAEEYEHAKKLIIFLNENNVPVQLTSISAPEHKFEGLTQIFQKAYEHE QHISESINNIVDHAIKSKDHATFNFLQWYVAEQHEEEVLFKDILDKIELIGN ENHGLYLADQYVKGIAKSRKSGS (SEQ ID NO: 1) or a fragment or variant thereof,
b. DIIKLLNEQVNKEMQSSNLYMSMSSWCYTHSLDGAGLFLFDHAAEEYEH AKKLIIFLNENNVPVQLTSISAPEHKFEGLTQIFQKAYEHEQHISESINNIVD HAIKSKDHATFNFLQWYVAEQHEEEVLFKDILDKIELIGNENHGLYLADQ YVKGIAKSRKSGS (SEQ ID NO: 2) or a fragment or variant thereof, and
c. SKDIIKLLNEQVNKEMQSSNLYMSMSSWCYTHSLDGAGLFLFDHAAEEY EHAKKLIIFLNENNVPVQLTSISAPEHKFEGLTQIFQKAYEHEQHISESINNI VDHAIKSKDHATFNFLQWYVAEQHEEEVLFKDILDKIELIGNENHGLYLA DQYVKGIAKSRKSGS (SEQ ID NO: 3) or a fragment or variant thereof.

13. The composition of any one of the preceding claims, wherein at least two said antigenic coronavirus peptides are comprised in a fusion protein, wherein the antigenic coronavirus peptides are connected via a linker.

14. The composition of any one of the preceding claims, wherein 2-10 said antigenic coronavirus peptides are comprised in a fusion protein in series, optionally wherein the antigenic coronavirus peptides are connected via a linker.

15. The composition of any one of claims 3-14, wherein an antigenic coronavirus peptide is comprised in a fusion protein with a nanoparticle-forming peptide, wherein the antigenic coronavirus peptide is connected to the nanoparticle-forming peptide via a linker.

16. The composition of any one of claims 13-15, wherein the linker comprises an amino acid sequence selected from: GGGSGGSG (SEQ ID NO: 583), GSGGGG (SEQ ID NO: 11), GGGG (SEQ ID NO: 15), GSGG (SEQ ID NO: 5), GGG (SEQ ID NO: 16), and SGG (SEQ ID NO: 17).

17. The composition of any one of the preceding claims, wherein first and second antigenic coronavirus peptides are comprised in a mosaic coronavirus spike protein, wherein at least one domain of the mosaic coronavirus spike protein is substituted or added from a heterologous coronavirus strain.

18. The composition of any one of claims 3-17 wherein an antigenic coronavirus peptide is comprised in a fusion protein with a nanoparticle-forming peptide, wherein the fusion protein(s) comprise one or more formats selected from SpFn, beads on a string, domain fusion, domain swap, loop insertion, and domain insertion.

19. The composition of any one of claims 3-18 wherein an antigenic coronavirus peptide is comprised in a fusion protein with a nanoparticle-forming peptide, wherein the fusion protein has a format selected from the formats shown in any one of FIGS. 2-7.

20. The composition of any one claims 3-19 wherein an antigenic coronavirus peptide is comprised in a fusion protein with a nanoparticle-forming peptide, wherein at least the first and second antigenic coronavirus peptides are different RBD peptides from different coronavirus strains (e.g., R₁, R₂) comprised in the same fusion protein (e.g., R₁R₂FN), optionally wherein the fusion protein further comprises a Spike protein (e.g., R₁R₂mosSpFN), further optionally wherein the composition comprises two or more different fusion proteins comprising the same two or more different RBD peptides in different positions in the fusion protein (e.g., R₁R₂FN, R₂R₁FN or R₁R₂mosSpFN, R₂R₁mosSpFN), or mRNA molecules encoding said two or more different fusion proteins, or a nanoparticle displaying said two or more different fusion proteins.

21. The composition of any one of the preceding claims, wherein the antigenic coronavirus peptides are comprised in one or more fusion proteins comprising an amino acid sequence selected from those disclosed in Table 6 or Table 7 (SEQ ID NOs: 29-551), or a sequence having at least 80% sequence identity thereto.

22. The composition of any one of claims 3-21, wherein the composition comprises a nanoparticle comprising the at least two antigenic coronavirus peptides.

23. The composition of any one of claims 1-21, wherein the composition comprises one or more mRNA molecules encoding the at least two antigenic coronavirus peptides, optionally wherein the one or mRNA molecules are encapsulated or co-encapsulated in one or more lipid nanoparticles (LNPs).

24. The composition of claim 23, selected from:
a. a composition comprising one mRNA molecule encoding one fusion protein comprising the at least two antigenic coronavirus peptides, optionally wherein the fusion protein further comprises a nanoparticle-forming peptide, wherein the mRNA molecule is encapsulated in a lipid nanoparticle (LNP);
b. a composition comprising two or more mRNA molecules, each encoding at least one of the at least two antigenic coronavirus peptides, optionally in a fusion protein with a nanoparticle-forming peptide, wherein each mRNA molecule is encapsulated in a separate lipid nanoparticle (LNP);
c. a composition comprising two or more mRNA molecules, each encoding at least one of the at least two antigenic coronavirus peptides, optionally in a fusion protein with a nanoparticle-forming peptide, wherein two or more mRNA molecules are co-encapsulated in the same lipid nanoparticle (LNP).

25. The composition of any one of the preceding claims comprising an mRNA molecule, wherein the mRNA molecule has one or more features selected from:
a. a 5′ untranslated region (5′ UTR);
b. a 3′ untranslated region (3′ UTR);
c. a polyadenylation (poly(A)) sequence;
d. a chemical modification, optionally wherein the chemical modification comprises N1-methylpseudouridine; and
e. the mRNA is a self-replicating mRNA or a non-replicating mRNA, optionally wherein the mRNA molecule is encapsulated in a lipid nanoparticle (LNP).

26. The composition of any one of the preceding claims, further comprising an adjuvant.

27. The composition of claim 26, wherein the composition comprises an antigen or nanoparticle and the adjuvant comprises one or more selected from ALFQ, alhydrogel, and combinations thereof.

28. A nanoparticle comprising a fusion protein comprising a nanoparticle-forming peptide and at least two antigenic coronavirus peptides independently selected from:
a. a receptor-binding domain (RBD) of a coronavirus, or a fragment or variant thereof,
b. an N-terminal domain (NTD) of a coronavirus, or a fragment or variant thereof,
c. an S1 domain of a coronavirus, or a fragment or variant thereof,
d. a stabilized extracellular spike S-2P domain of a coronavirus, or a fragment or variant thereof,
e. a stabilized extracellular spike S domain of a coronavirus, or a fragment or variant thereof,
f. a stabilized extracellular spike S-trimer of a coronavirus, or a fragment or variant thereof, and
g. a mosaic coronavirus spike protein, wherein at least one domain of the mosaic coronavirus spike protein is substituted or added from a heterologous coronavirus strain.

29. A DNA molecule comprising a sequence encoding a nanoparticle according to claim 28, or a plasmid comprising said DNA molecule, optionally wherein the plasmid can express the DNA molecule in vivo.

30. A method of treating or preventing a coronavirus infection in a subject in need thereof, comprising administering to a subject in need thereof the immunogenic composition of any one of claims 1-29.

31. The immunogenic composition of any one of claims 1-29, for use in treating or preventing a coronavirus infection in a subject in need thereof.

32. Use of the immunogenic composition of any one of claims 1-29 in the preparation of a medicament for treating or preventing a coronavirus infection in a subject in need thereof.

33. The method, composition for use, or use of any one of claims 30-32, wherein the subject is at risk of contracting a coronavirus infection.

34. The method, composition for use, or use of any one of claims 30-32, wherein the subject has already contracted a coronavirus infection.

35. The method, composition for use, or use of any one of claims 30-34, wherein the subject has not previously been administered a vaccine for prevention of a coronavirus infection.

36. The method, composition for use, or use of any one of claims 30-34, wherein the subject has previously been administered one or more vaccines for the prevention of a coronavirus infection.

37. The method, composition for use, or use of any one of claims 30-36, wherein the method elicits an immune response in the subject against a coronavirus, optionally wherein the immune response comprises neutralizing antibodies, further optionally wherein the neutralizing antibodies cross-neutralizes two or more coronavirus strains, further optionally wherein the neutralizing antibodies cross-neutralizes one or more coronavirus strains that is not a component strain of the immunogenic composition.

38. An mRNA molecule comprising or consisting of a sequence selected from any one of SEQ ID NOs: 552-582 or a sequence having at least 80%, at least 85%, at least 90%, at least 95%, at least 96%, at least 97%, at least 98%, or at least 99% homology or identity thereto.
